(19) United States
(12) Patent Application Publication
(10) Pub. No.: US 2022/0315598 A1
(43) Pub. Date: Oct. 6, 2022

(54) Oxygen-containing Heterocyclic Compound, Preparation Method Therefor and Use Thereof

(71) Applicant: SHANGHAI YINGLI PHARMACEUTICAL CO., LTD, Shanghai (CN)

(72) Inventors: Zusheng XU, Toronto (CA); Yangtong LOU, Shanghai (CN); Tiegang XIE, Shanghai (CN)

(21) Appl. No.: 17/767,046

(22) PCT Filed: Oct. 21, 2020
(86) PCT No.: PCT/CN2020/122335

§ 371 Date: Apr. 6, 2022

(30) Foreign Application Priority Data
Dec. 2, 2019 (CN) 201911212840.3
Apr. 29, 2020 (CN) 202010368798.0
Oct. 10, 2020 (CN) 202011077052.0

(51) Int. Cl. C07D 491/052; A61P 35/00; C07D 519/00

(52) U.S. Cl. CPC C07D 491/052; A61P 35/00; C07D 519/00; C07B 2200/07

(57) ABSTRACT

Disclosed are an oxygen-containing heterocyclic compound, a preparation method therefor and the use thereof. The present disclosure provides an oxygen-containing heterocyclic compound represented by formula I, a pharmaceutically acceptable salt thereof, a stereoisomer thereof, a tautomer thereof or an isotopic compound thereof. It is expected that the oxygen-containing heterocyclic compound can treat and/or prevent various Ras-mediated diseases.

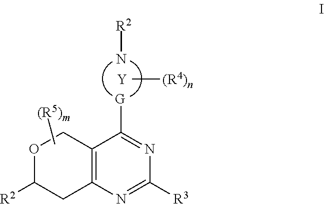

The present application claims the priority of Chinese patent application 201911212840.3 filed on Dec. 2, 2019, Chinese patent application 202010368798.0 filed on Apr. 29, 2020, and Chinese patent application 202011077052.0 filed on Oct. 10, 2020. The contents of the aforementioned Chinese patent applications are incorporated into the present application by reference in their entirety.

### TECHNICAL FIELD

The present disclosure relates to an oxygen-containing heterocyclic compound, a preparation method therefor and use thereof.

### BACKGROUND

Ras (Rat sarcoma viral oncogene) was first identified in rat sarcoma. In mammals, the ras gene family is composed of three members: H-ras, K-ras and N-ras, and the fourth exon of K-ras has two variants: A and B. Ras genes are widely present in various eukaryotes, such as mammals. Drosophila, fungi, nematodes and yeast, and are expressed at varying degrees in different tissues. H-Ras is mainly expressed in skin and skeletal muscle: K-Ras is mainly expressed in colon and thymus; and N-Ras is highly expressed in testis. Ras protein acts as a molecular switch in signal transduction of cells and regulates signal transduction by binding GTP/GDP and exchanging between them, thereby regulating life processes such as cell proliferation, differentiation, aging and apoptosis.

Ras mutation is closely related to the occurrence and development of tumors. Ras gene is mutated in at least 30% of human tumors and is considered to be one of the most powerful drivers of cancer. Mutations in the Ras proto-oncogenes are mainly point mutations. More than 150 different Ras point mutations have been found, most of which are mutations at positions 12 and 13 of glycine and position 61 of glutamine.

For decades, efforts have been made to research and develop a small molecule inhibitor targeting Ras, but so far, no related drugs have been marketed. Scientists have always wanted to develop a competitive inhibitor of GTP that directly acts on Ras proteins, but without success, due to strong affinity between GTP and Ras (pmol/L level), high concentration of GTP in cells (0.5 mM), lack of pockets that are conducive to the binding of small molecules in the RAS protein structure, etc. In recent years, people have made some progress in drug research and development using allosteric sites of the K-Ras G12C mutant. In 2013, a research team reported the discovery of K-Ras G12C small molecule inhibitors (Nature, 2013, 503, 548-551) and they identified a new binding pocket, i.e., an allosteric pocket, located below molecular switch II region from the K-Ras G12C mutant, wherein these inhibitors bind to the allosteric pocket and form a covalent bond with nearby Cys12, thereby selectively inhibiting the activation of K-Ras G12C. Other researchers have reported KRAS inhibitors with cellular activity (Science, 2016, 351, 604-608). Currently, there are several drugs in clinical development. In 2018, Amgen began clinical trials on compound AMG510, which is the first small molecule inhibitor that directly targets KRAS to enter the clinic.

In summary, after decades of unremitting efforts, people have gradually deepened their understanding of Ras, but no medicinal compounds have been successfully developed so far. Finding compounds with better inhibitory effects on Ras is still a research hotspot and difficulty in the field of new drug development.

### Content of the Present Invention

The technical problem to be solved in the present disclosure is that there is no effective drug in the prior art as an Ras inhibitor for clinical treatment, and therefore, the present disclosure provides an oxygen-containing heterocyclic compound, a preparation method therefore and use thereof, wherein the oxygen-containing heterocyclic compound is expected to treat and/or prevent various Ras-mediated diseases.

The present disclosure solves the above-mentioned technical problem through the following technical solutions.

The present disclosure provides an oxygen-containing heterocyclic compound represented by formula I, a pharmaceutically acceptable salt thereof, a stereoisomer thereof, a tautomer thereof or an isotopic compound thereof.

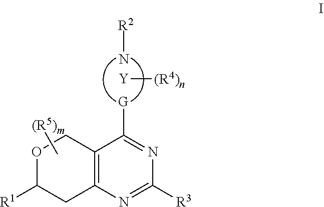

wherein R¹ is C₆₋₂₀ aryl, “5-12 membered heteroaryl containing 1-4 heteroatoms selected from one or more of O, S and N”. C₆₋₂₀ aryl substituted with one or more R¹⁻⁶, or “5-12 membered heteroaryl containing 1-4 heteroatoms selected from one or more of O, S and N” substituted with one or more R¹⁻⁷;

R¹⁻⁶ and R¹⁻⁷ are independently halogen, hydroxyl, —C(═O)R⁶⁵, —NR⁶³R⁶⁴, —C(═O)OR⁶⁶, —C(═O)NR⁶⁹R⁶¹⁰. C₁₋₆ alkyl, C₁₋₆ alkoxy. C₃₋₁₀ cycloalkyl. “5-7 membered heterocycloalkyl containing 1 or 2 heteroatoms selected from one or more of O and N”, C₆₋₂₀ aryl. “5-7 membered heteroaryl containing 1 or 2 heteroatoms selected from one or more of O and N”. C₁₋₆ alkyl substituted with one or more R¹⁻⁶⁻¹, C₁₋₆ alkoxy substituted with one or more R¹⁻⁶⁻². C₃₋₁₀ cycloalkyl substituted with one or more R¹⁻⁶⁻³. “5-7 membered heterocycloalkyl containing 1 or 2 heteroatoms selected from one or more of O and N” substituted with one or more R¹⁻⁶⁻⁴. C₆₋₂₀ aryl substituted with one or more R¹⁻⁶⁻⁵, or “5-7 membered heteroaryl containing 1 or 2 heteroatoms selected from one or more of O and N” substituted with one or more R¹⁻⁶⁻⁶;

R¹⁻⁶⁻¹, R¹⁻⁶⁻², R¹⁻⁶⁻³, R¹⁻⁶⁻⁴, R¹⁻⁶⁻⁵ and R¹⁻⁶⁻⁶ are independently cyano, halogen, hydroxyl. C₁₋₆ alkoxy. C₁₋₆ alkyl, —C(═O)R⁶⁵⁻², —NR⁶³⁻²R⁶⁴⁻², —C(═O)OR⁶⁶⁻², or —C(═O)NR⁶⁹⁻²R⁶¹⁰⁻²;

R⁶⁵, R⁶⁵⁻², R⁶³, R⁶³⁻², R⁶⁴, R⁶⁴⁻², R⁶⁶, R⁶⁶⁻², R⁶⁹, R⁶⁹⁻², R⁶¹⁰ and R⁶¹⁰⁻² are independently hydrogen or C₁₋₆ alkyl;

m is 0, 1 or 2;

R⁵ is independently C₁₋₆ alkyl;

R³ is —OR³¹, —SR³² or —NR³³R³⁴;

R³¹, R³² and R³⁴ are independently C₁₋₆ alkyl substituted with one or more R³¹⁻¹; R³³ is independently H, C₁₋₆ alkyl, or C₁₋₆ alkyl substituted with one or more R³¹⁻¹;

R³¹⁻¹ is independently C₃₋₁₀ cycloalkyl, “4-10 membered heterocycloalkyl containing 1-3 heteroatoms selected from one or more of O and N”, C₃₋₁₀ cycloalkyl substituted with one or more R^{d16}. “4-10 membered heterocycloalkyl containing 1-3 heteroatoms selected from one or more of O and N” substituted with one or more R^{d15}, —OR^{d}, —SR^{d1}, —NR^{e1}R^{e2}, or —C(═O)NR^{e3}R^{e4};

R^{d15} and R^{d16} are independently C₁₋₆ alkyl, C₁₋₆ alkyl substituted with one or more R¹⁻⁸⁻¹, hydroxyl, C₁₋₆ alkoxy, halogen, —NR^{e5}R^{e6} or —C(═O)NR^{e7}R^{e8};

R^{d}, R^{d1}, R^{e1}, R^{e2} and R^{e3} are independently hydrogen, C₁₋₆ alkyl, C₃₋₁₀ cycloalkyl, “4-10 membered heterocycloalkyl containing 1-3 heteroatoms selected from one or more of 0 and N”, or C₁₋₆ alkyl substituted with one or more R¹⁻⁸⁻²;

R¹⁻⁸⁻¹ and R¹⁻⁸⁻² are independently cyano, halogen, hydroxyl, C₁₋₆ alkoxy, —C(═O)R^{e9}, —NR^{e10}R^{e11}, —C(═O)OR^{e12}, or —C(═O)NR^{e13}R^{e14};

R^{e5}, R^{e6}, R^{e7}, R^{e8}, R^{e9}, R^{e10}, R^{e11}, R^{e12}, R^{e13} and R^{e14} are independently hydrogen or C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring, a bridged ring or a spiro ring;

G is N, C or CH;

n is 0, 1, 2 or 3;

R⁴ is independently C₁₋₆ alkyl, C₁₋₆ alkyl substituted with one or more R⁴⁻¹, oxo, —C(═O)OR^{4a} or —C(═O)NR^{4b}R^{4c};

R⁴⁻¹ is independently halogen, cyano, hydroxyl, C₁₋₆ alkoxy, —NR⁴ⁱR^{4j}, —C(═O)OR^{4d} or —C(═O)OR^{4e}R^{4f}; R^{4d}, R^{4e}, R^{4f}, R⁴ⁱ and R^{4j} are independently hydrogen or C₁₋₆ alkyl;

R^{4a}, R^{4b} and R^{4c} are independently hydrogen or C₁₋₆ alkyl;

R² is CN, —C(═O)—C(R^{a})═C(R^{b})(R^{f}), —C(═O)—C≡R^{f}, —S(═O)₂—C(R^{a})═C(R^{b})(R^{f}) or —S(═O)₂—C≡CR^{f};

R^{a} is independently hydrogen, deuterium, halogen or C₁₋₆ alkyl;

R^{b} and R^{f} are independently hydrogen, deuterium, C₁₋₆ alkyl, C₁₋₆ alkyl-C(═O)—, or C₁₋₆ alkyl substituted with one or more R^{b-1};

R^{b-1} is independently halogen, hydroxyl, C₁₋₆ alkoxy, or —NR^{10j}R^{10k};

R^{10j} and R^{10k} are independently hydrogen or C₁₋₆ alkyl, or R^{10j} and R^{10k} taken together with the N atom to which they are attached form “4-10 membered heterocycloalkyl containing 1-3 heteroatoms selected from one or more of O and N”.

In a certain embodiment, with regard to an oxygen-containing heterocyclic compound represented by formula I, a pharmaceutically acceptable salt thereof, a stereoisomer thereof, a tautomer thereof or an isotopic compound thereof, some groups are as defined as follows, and the unmentioned group definitions are as described in any one of the embodiments of the present disclosure (this content is hereinafter referred to simply as “in a certain embodiment”). With regard to an oxygen-containing heterocyclic compound represented by formula I.

R¹ is C₆₋₂₀ aryl. “5-12 membered heteroaryl containing 1-4 heteroatoms selected from one or more of O, S and N”. C₆₋₂₀ aryl substituted with one or more R¹⁻⁶, or “5-12 membered heteroaryl containing 1-4 heteroatoms selected from one or more of O, S and N” substituted with one or more R¹⁻⁷;

R¹⁻⁶ and R¹⁻⁷ are independently halogen, hydroxyl, —C(═O)R⁶⁵, —NR⁶³R⁶⁴, —C(═O)OR⁶⁶, —C(═O)NR⁶⁹R⁶¹⁰, C₁₋₆ alkyl, C₁₋₆ alkoxy, C₃₋₁₀ cycloalkyl. “5-7 membered heterocycloalkyl containing 1 or 2 heteroatoms selected from one or more of O and N”, C₆₋₂₀ aryl. “5-7 membered heteroaryl containing 1 or 2 heteroatoms selected from one or more of O and N”. C₁₋₆ alkyl substituted with one or more alkoxy substituted with one or more R¹⁻⁶⁻². C₃₋₁₀ cycloalkyl substituted with one or more R¹⁻⁶⁻³, “5-7 membered heterocycloalkyl containing 1 or 2 heteroatoms selected from one or more of O and N” substituted with one or more R¹⁻⁶⁻⁴, C₆₋₂₀ aryl substituted with one or more R¹⁻⁶⁻⁵, or “5-7 membered heteroaryl containing 1 or 2 heteroatoms selected from one or more of O and N” substituted with one or more R¹⁻⁶⁻⁶;

R¹⁻⁶⁻¹, R¹⁻⁶⁻², R¹⁻⁶⁻³, R¹⁻⁶⁻⁴, R¹⁻⁶⁻⁵ and R¹⁻⁶⁻⁶ are independently cyano, halogen, hydroxyl, C₁₋₆ alkoxy. C₁₋₆ alkyl, —C(═O)R⁶⁵⁻², —NR⁶³⁻², R⁶⁴⁻², —C(═O)OR⁶⁶⁻², or —C(═O)NR⁶⁹⁻²R⁶¹⁰⁻²;

R⁶⁵, R⁶⁵⁻², R⁶³, R⁶³⁻², R⁶⁴, R⁶⁴⁻², R⁶⁶, R⁶⁶⁻², R⁶⁹, R⁶⁹⁻², R⁶¹⁰ and R⁶¹⁰⁻² are independently hydrogen or C₁₋₆ alkyl;

m is 0, 1 or 2;

R⁵ is independently C₁₋₆ alkyl;

R³ is —OR³¹, —SR³² or —NR³³R³⁴;

R³¹, R³², R³³ and R³⁴ are independently C₁₋₆ alkyl substituted with one or more R³¹⁻¹;

R³¹⁻¹ is independently C₃₋₁₀ cycloalkyl, “4-10 membered heterocycloalkyl containing 1-3 heteroatoms selected from one or more of O and N”. C₃₋₁₀ cycloalkyl substituted with one or more R^{d16}. “4-10 membered heterocycloalkyl containing 1-3 heteroatoms selected from one or more of O and N” substituted with one or more R^{d15}, —OR^{d}, —SR^{d1}, —NR^{e1}R^{e2}, or —C(═O)NR^{e3}R^{e4};

R^{d15} and R^{d16} are independently C₁₋₆ alkyl, C₁₋₆ alkyl substituted with one or more R¹⁻⁸⁻¹, hydroxyl, C₁₋₆ alkoxy, halogen, —NR^{e5}R^{e6} or —C(═O)NR^{e7}R^{e8};

R^{d}, R^{d1}, R^{e1}, R^{e2}, R^{e3} and R^{e4} are independently hydrogen, C₁₋₆ alkyl, C₃₋₁₀ cycloalkyl, “4-10 membered heterocyclo alkyl containing 1-3 heteroatoms selected from one or more of O and N”, or C₁₋₆ alkyl substituted with one or more R¹⁻⁸⁻²;

R¹⁻⁸⁻¹ and R¹⁻⁸⁻² are independently cyano, halogen, hydroxyl, C₁₋₆ alkoxy, —C(═O)R^{e9}. NR^{e10}R^{e11}, —C(═O)OR^{e12}, or —C(═O)NR^{e13}R^{e14};

R^{e5}, R^{e6}, R^{e7}, R^{e8}, R^{e9}, R^{e10}, R^{e11}, R^{e12}, R^{e13} and R^{e14} are independently hydrogen or C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring, a bridged ring or a spiro ring;

G is N, C or CH;

n is 0, 1, 2 or 3;

R⁴ is independently C₁₋₆ alkyl, C₁₋₆ alkyl substituted with one or more R⁴⁻¹, oxo, —C(═O)OR^{4a} or —C(═O)NR^{4b}R^{4c};

R⁴⁻¹ is independently halogen, cyano, hydroxyl, C₁₋₆ alkoxy, —C(═O)OR^{4d} or —C(═O)NR^{4e}R^{4f}; R^{4d}, R^{4e}, R^{4f}, R⁴ⁱ and R^{4j} are independently hydrogen or C₁₋₆ alkyl;

R^{4a}, R^{4b} and R^{4c} are independently hydrogen or C₁₋₆ alkyl;

R² is —C(═O)—C(R^{a})═C(R^{b})(R^{f}), —C(═O)—C≡CR^{f}, —S(═O)₂—C(R^{a})═C(R^{b})(R^{f}) or —S(═O)₂—C≡CR^{f};

R^{a} is independently hydrogen, deuterium, halogen or C₁₋₆ alkyl;

R^{b} and R^{f} are independently hydrogen, deuterium, C₁₋₆ alkyl, or C₁₋₆ alkyl substituted with one or more R^{b-1};

R^{b-1} is independently halogen, hydroxyl, C₁₋₆ alkoxy, or —NR^{10j}R^{10k};

R^{10j} and R^{10k} are independently hydrogen or C₁₋₆ alkyl, or R^{10j} and R^{10k} taken together with the N atom to which they are attached form “4-10 membered heterocycloalkyl containing 1-3 heteroatoms selected from one or more of O and N”.

In a certain embodiment;

R¹ is C₆₋₂₀ aryl. “5-12 membered heteroaryl containing 1-4 heteroatoms selected from one or more of O, S and N”. C₆₋₂₀ aryl substituted with one or more R¹⁻⁶, or “5-12 membered heteroaryl containing 1-4 heteroatoms selected from one or more of O, S and N” substituted with one or more R¹⁻⁷;

R¹⁻⁶ and R¹⁻⁷ are independently halogen, hydroxyl, C₁₋₆ alkyl, C₁₋₆ alkoxy. C₃₋₁₀ cycloalkyl, C₁₋₆ alkyl substituted with one or more R¹⁻⁶⁻¹, or C₁₋₆ alkoxy substituted with one or more R¹⁻⁶⁻²; R¹⁻⁶⁻¹ and R¹⁻⁶⁻² are independently halogen.

In a certain embodiment;

R¹ is C₆₋₂₀ aryl. “5-12 membered heteroaryl containing 1-4 heteroatoms selected from one or more of O, S and N”, C₆₋₂₀ aryl substituted with one or more R¹⁻⁶, or “5-12 membered heteroaryl containing 1-4 heteroatoms selected from one or more of O, S and N” substituted with one or more R¹⁻⁷; for example, C₆₋₂₀ aryl. “5-12 membered heteroaryl containing 1-4 heteroatoms selected from one or more of O, S and N”, or C₆₋₂₀ aryl substituted with one or more R¹⁻⁶;

R¹⁻⁶ and R¹⁻⁷ are independently halogen. C₁₋₆ alkyl, or C₁₋₆ alkyl substituted with one or more R¹⁻⁶⁻¹;

R¹⁻⁶⁻¹ is independently halogen.

In a certain embodiment;

R² is C₆₋₂₀ aryl, or C₆₋₂₀ aryl substituted with one or more R¹⁻⁶;

R¹⁻⁶ is independently halogen. C₁₋₆ alkyl, or C₁₋₆ alkyl substituted with one or more R¹⁻⁶⁻¹;

R¹⁻⁶⁻¹ is independently halogen.

In a certain embodiment;

m is 0.

In a certain embodiment;

R³ is —OR³¹, —SR³² or —NR³³R³⁴;

R³¹, R³² and R³⁴ are independently C₁₋₆ alkyl substituted with one or more R³¹⁻¹; R³³ is independently H, C₁₋₆ alkyl, or C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl or halogen;

R^{e1} and R^{e2} are independently C₁₋₆ alkyl.

In a certain embodiment;

R³ is —SR³²;

R³² is C₁₋₆ alkyl substituted with one or more R³¹⁻¹;

R³¹⁻¹ is independently —NR^{e1}R^{e2};

R^{e1} and R^{e2} are independently C₁₋₆ alkyl.

In a certain embodiment;

R³ is —OR³¹ or —NR³³R³⁴;

R³¹, R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl, C₁₋₆ alkyl substituted with one or more R¹⁻⁸⁻¹, hydroxyl. C₁₋₆ alkoxy, halogen, —NR^{e5}R^{e6} or —C(═O)NR^{e7}R^{e8};

R¹⁻⁸⁻¹ is independently halogen; R^{e5}, R^{e6}, R^{e7} and R^{e8} are independently hydrogen or C₁₋₆ alkyl;

R^{e1} and are independently C₁₋₆ alkyl;

In a certain embodiment;

R³ is —NR³³R³⁴;

R³⁴ is independently C₁₋₆ alkyl substituted with one or more R³¹⁻¹; R³³ is independently H, C₁₋₆ alkyl, or C₁₋₆ alkyl substituted with one or more R³¹⁻¹; for example H or C₁₋₆ alkyl;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl;

R^{e1} and R^{e2} are independently C₁₋₆ alkyl.

In a certain embodiment;

R³ is —OR³¹ or —NR³³R³⁴;

R³¹ and R³⁴ are independently C₁₋₆ alkyl substituted with one or more R³¹⁻¹; R³³ is independently H, C₁₋₆ alkyl, or C₁₋₆ alkyl substituted with one or more R³¹⁻¹; for example H or C₁₋₆ alkyl;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl or halogen;

R^{e1} and R^{e2} are independently C₁₋₆ alkyl.

In a certain embodiment;

R³ is —OR³¹ or —NR³³R³⁴;

R³¹, R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl or halogen;

R^{e1} and R^{e2} are independently C₁₋₆, alkyl.

In a certain embodiment;

R³ is —OR³¹ or —NR³³R³⁴;

R³¹, R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{e1}, R^{e2} and R^{d15} are independently C₁₋₆ alkyl.

In a certain embodiment;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring or a spiro ring; for example, a monocyclic ring;

G is N, C or CH.

In a certain embodiment;

ring Y is a 4-12 membered heterocyclic ring containing 1-4 N atoms; the heterocyclic rung is a saturated heterocyclic ring or a partly saturated heterocyclic ring; the heterocyclic ring is a monocyclic ring;

G is N.

In a certain embodiment;

n is 0 or 1;

R⁴ is independently C₁₋₆ alkyl, or C₁₋₆ alkyl substituted with one or more R⁴⁻¹;

R⁴⁻¹ is independently hydroxyl, cyano, or —C(═O)NR^{4e}R^{4f}; R^{4e} and R^{4f} are independently hydrogen or C₁₋₆ alkyl; for example, hydrogen.

In a certain embodiment;

n is 0 or 1;

R⁴ is independently C₁₋₆ alkyl substituted with one or more R⁴⁻¹;

R⁴⁻¹ is independently —C(═O)NR^{4e}R^{4f}; R^{4e} and R^{4f} are independently hydrogen or C₁₋₆ alkyl; for example, hydrogen.

In a certain embodiment;

n is 0 or 1;

R⁴ is independently C₁₋₆ alkyl, or C₁₋₆ alkyl substituted with one or more R⁴⁻¹;

R⁴⁻¹ is independently hydroxyl or cyano.

In a certain embodiment;

n is 0 or 1;

R⁴ is independently C₁₋₆ alkyl, or C₁₋₆ alkyl substituted with one or more R⁴⁻¹;

R⁴⁻¹ is independently hydroxyl.

In a certain embodiment;

n is 0 or 1;

R⁴ is independently C₁₋₆ alkyl, or C₁₋₆ alkyl substituted with one or more R⁴⁻¹;

R⁴⁻¹ is independently cyano.

In a certain embodiment;

R² is CN, —C(═O)—C(R^{a})═C(R^{b})(R^{f}), —C(═O)—C≡C-Me or, —S(═O)₂—C(R^{a})═C(R^{b})(R^{f});

R^{a} is independently hydrogen or halogen;

R^{b} and R^{f} are independently hydrogen, C₁₋₆ alkyl, C₁₋₆ alkyl-C(═O)—, or C₁₋₆ alkyl substituted with one or more R^{b-1};

R^{b-1} is independently —NR^{10j}R^{10k};

R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

In a certain embodiment;

R² is CN or —C(═O)—C(R²)═C(R^{b})(R^{f});

R^{a} is independently hydrogen;

R^{b} and R^{f} are independently hydrogen, C₁₋₆ alkyl-C(═O)—.

In a certain embodiment;

R² is —C(═O)—C(R^{a})═C(R^{b})(R^{f}), —C(═O)—C≡C-Me or —S(═O)₂—C(R^{a})═C(R^{b})(R^{f});

R^{a} is independently hydrogen or halogen;

R^{b} and R^{f} are independently hydrogen, C₁₋₆ alkyl, or C₁₋₆ alkyl substituted with one or more R^{b-1};

R^{b-1} is independently halogen, hydroxyl, C₁₋₆ alkoxy, or —NR^{10j}R^{10k};

R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

In a certain embodiment;

R² is —C(═O)—C(R^{a})═C(R^{b})(R^{f}), —C(═O)—C≡C-Me or —S(═O)₂—C(R^{a})═C(R^{b})(R^{f});

R^{a} is independently hydrogen or halogen;

R^{b} and R^{f} are independently hydrogen, C₁₋₆ alkyl, or C₁₋₆ alkyl substituted with one or more R^{b-1};

R^{b-1} is independently —NR^{10j}R^{10k};

R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

In a certain embodiment;

R¹ is C₆₋₂₀ aryl. “5-12 membered heteroaryl containing 1-4 heteroatoms selected from one or more of O, S and N”. C₆₋₂₀ aryl substituted with one or more R¹⁻⁶, or “5-12 membered heteroaryl containing 1-4 heteroatoms selected from one or more of O, S and N” substituted with one or more R¹⁻⁷;

R¹⁻⁶ and R¹⁻⁷ are independently halogen. C₁₋₆ alkyl, C₁₋₆ alkyl substituted with one or more R¹⁻⁶⁻¹; R¹⁻⁶⁻¹ is independently halogen;

m is 0;

R³ is —OR³¹, —SR³² or —NR³³R³⁴;

R³¹, R³² and R³⁴ are independently C₁₋₆ alkyl substituted with one or more R³¹⁻¹; R³³ is independently H, C₁₋₆ alkyl, or C₁₋₆ alkyl substituted with one or more R³¹⁻¹; for example H or C₁₋₆ alkyl;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl or halogen;

R^{e1} and R^{e2} are independently C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring or a spiro ring;

G is N, C or CH;

n is 0 or 1;

R⁴ is independently C₁₋₆ alkyl, or C₁₋₆ alkyl substituted with one or more R⁴⁻¹;

R⁴⁻¹ is independently cyano, hydroxyl or —C(═O)NR^{4e}R^{4f}, R^{4e} and R^{4f} are independently hydrogen or C₁₋₆ alkyl;

R² is CN, —C(═O)—C(R^{a})═C(R^{b})(R^{f}), —C(═O)—C≡C-Me or, —S(═O)₂—C(R^{a})═C(R^{b})(R^{f});

R^{a} is independently hydrogen or halogen;

R^{b} and R^{f} are independently hydrogen, C₁₋₆ alkyl, C₁₋₆ alkyl-C(═O)—, or C₁₋₆ alkyl substituted with one or more R^{b-1};

R^{b-1} is independently halogen, hydroxyl, C₁₋₆ alkoxy, or —NR^{10j}R^{10k};

R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

In a certain embodiment;

R¹ is C₆₋₂₀ aryl, “5-12 membered heteroaryl containing 1-4 heteroatoms selected from one or more of O, S and N”. C₆₋₂₀ aryl substituted with one or more R¹⁻⁶, or “5-12 membered heteroaryl containing 1-4 heteroatoms selected from one or more of O, S and N” substituted with one or more R¹⁻⁷;

R¹⁻⁶ and R¹⁻⁷ are independently halogen, hydroxyl, C₁₋₆ alkyl, C₁₋₆ alkoxy. C₃₋₁₀ cycloalkyl, C₁₋₆ alkyl substituted with one or more R¹⁻⁶⁻¹, or C₁₋₄ alkoxy substituted with one or more R¹⁻⁶⁻²; R¹⁻⁶⁻¹ and R¹⁻⁶⁻² are independently halogen;

m is 0;

R³ is —OR³¹ or —NR³³R³⁴;

R³¹, R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl, C₁₋₆ alkyl substituted with one or more R¹⁻⁹⁻¹, hydroxyl, C₁₋₆ alkoxy, halogen, —NR^{e5}R^{e6} or —C(═O)NR^{e7}R^{e8};

R¹⁻⁸⁻¹ is independently halogen; R^{e5}, R^{e6}, R^{e7} and R^{e8} are independently hydrogen or C₁₋₆ alkyl;

R^{e1} and R^{e2} are independently C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring or a spiro ring;

G is N, C or CH;

n is 0 or 1;

R⁴ is independently C₁₋₆ alkyl, or C₁₋₆ alkyl substituted with one or more R⁴⁻¹;

R⁴⁻¹ is independently cyano or hydroxyl;

R² is —C(═O)—C(R^{a})═C(R^{b})(R^{f}), —C(═O)—C≡C-Me or —S(═O)₂—C(R²)═C(R^{b})(R^{f});

R^{a} is independently hydrogen or halogen;

R^{b} and R^{f} are independently hydrogen, C₁₋₆ alkyl, or C₁₋₆ alkyl substituted with one or more R^{b-1};

R^{b-1} is independently halogen, hydroxyl, C₁₋₆ alkoxy, or —NR^{10j}R^{10k};

R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

In a certain embodiment;

R² is —C(═O)—C(R^{a})═C(R^{b})(R^{f}); R^{a} is independently hydrogen or halogen; R^{b} and R^{f} are independently hydrogen or C₁₋₆ alkyl.

In a certain embodiment;

R¹ is C₆₋₂₀ aryl. “5-12 membered heteroaryl containing 1-4 heteroatoms selected from one or more of O, S and N”, C₆₋₂₀ aryl substituted with one or more R¹⁻⁶, or “5-12 membered heteroaryl containing 1-4 heteroatoms selected from one or more of O, S and N” substituted with one or more R¹⁻⁷;

R¹⁻⁶ and R¹⁻⁷ are independently halogen, hydroxyl, C₁₋₆ alkyl, C₁₋₆ alkoxy. C₃₋₁₀ cycloalkyl, C₁₋₆ alkyl substituted with one or more R¹⁻⁶⁻¹, or C₁₋₆ alkoxy substituted with one or more R¹⁻⁶⁻²; R¹⁻⁶⁻¹ and R¹⁻⁶⁻² are independently halogen;

m is 0;

R³ is —OR³¹ or —NR³³R³⁴;

R³¹, R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl, C₁₋₆ alkyl substituted with one or more R¹⁻⁸⁻¹, hydroxyl, C₁₋₆ alkoxy, halogen, —NR^{e5}R^{e6} or —C(═O)NR^{e7}R^{e8};

R¹⁻⁸⁻¹ is independently halogen; R^{e5}, R^{e6}, R^{e7} and R^{e8} are independently hydrogen or C₁₋₆ alkyl;

R^{e1} and R^{e2} are independently C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic rung is a monocyclic ring or a spiro ring;

G is N, C or CH;

n is 0 or 1;

R³ is independently C₁₋₆ alkyl, or C₁₋₆ alkyl substituted with one or more R⁴⁻¹;

R⁴⁻¹ is independently cyano;

R² is —C(═O)—C(R³)═C(R^{b})(R^{f}), —C(═O)—C≡C-Me or —S(═O)₂—C(R^{a})═C(R^{b})(R^{f});

R^{a} is independently hydrogen or halogen;

R^{b} and R^{f} are independently hydrogen, C₁₋₆ alkyl, or C₁₋₆ alkyl substituted with one or more R^{b-1};

R^{b-1} is independently halogen, hydroxyl, C₁₋₆ alkoxy, or —NR^{10j}R^{10k};

R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

In a certain embodiment;

R¹ is C₆₋₂₀ aryl, “5-12 membered heteroaryl containing 1-4 heteroatoms selected from one or more of O, S and N”. C₆₋₂₀ aryl substituted with one or more R¹⁻⁶, or “5-12 membered heteroaryl containing 1-4 heteroatoms selected from one or more of O, S and N” substituted with one or more R¹⁻⁷; for example, C₆₋₂₀ aryl. “5-12 membered heteroaryl containing 1-4 heteroatoms selected from one or more of O, S and N”, or C₆₋₂₀ aryl substituted with one or more R¹⁻⁶;

R¹⁻⁶ is independently halogen, C₁₋₆ alkyl, or C₁₋₆ alkyl substituted with one or more) R¹⁻⁶⁻¹ is independently halogen;

m is 0;

R³ is —OR³¹, —SR³², or —NR³³R³⁴;

R³¹, R³² and R³⁴ are independently C₁₋₆ alkyl substituted with one or more R³¹⁻¹; R³³ is independently H, C₁₋₆ alkyl, or C₁₋₆ alkyl substituted with one or more R³¹⁻¹; for example H or C₁₋₆ alkyl;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl or halogen;

R^{e1} and R^{e2} are independently C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring;

G is N;

n is 0 or 1;

R⁴ is independently C₁₋₆ alkyl substituted with one or more R⁴⁻¹;

R⁴⁻¹ is independently cyano or —C(═O)NR^{4e}R^{4f}; R^{4e} and R^{4f} are independently hydrogen or C₁₋₆ alkyl;

R² is CN, —C(═O)—C(R^{a})═C(R^{b})(R^{f});

R² is independently hydrogen or halogen;

R^{b} and R^{f} are independently hydrogen, C₁₋₆ alkyl, or C₁₋₆ alkyl-C(═O)—.

In a certain embodiment:

R¹ is C₆₋₂₀ aryl, or C₆₋₂₀ aryl substituted with one or more R¹⁻⁶;

R¹⁻⁶ is independently halogen. C₁₋₆ alkyl, or C₁₋₆ alkyl substituted with one or more R¹⁻⁶⁻¹;

R¹⁻⁶⁻¹ is independently halogen;

m is 0;

R³ is —OR³¹ or —NR³³R³⁴;

R³¹, R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl or halogen;

R^{e1} and R^{e2} are independently C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring;

G is N;

n is 0 or 1;

R⁴ is independently C₁₋₆ alkyl, or C₁₋₆ alkyl substituted with one or more R⁴⁻¹;

R⁴⁻¹ is independently cyano;

R² is —C(═O)—C(R^{a})═C(R^{b})(R^{f});

R^{a} is independently hydrogen or halogen;

R^{b} and R^{f} are independently hydrogen or C₁₋₆ alkyl, or C₁₋₆ alkyl substituted with one or more R^{b-1};

R^{b-1} is independently —NR^{10j}R^{10k};

R^{10j} and R^{10k} are independently hydrogen or C₁₋₆ alkyl, or R^{10j} and R^{10k} taken together with the N atom to which they are attached form “4-10 membered heterocycloalkyl containing 1-3 heteroatoms selected from one or more of O and N”.

In a certain embodiment:

R¹ is C₆₋₂₀ aryl, or C₆₋₂₀ aryl substituted with one or more R¹⁻⁶;

R¹⁻⁶ is independently halogen, C₁₋₆ alkyl, or C₁₋₆ alkyl substituted with one or more R¹⁻⁶⁻¹;

R¹⁻⁶⁻¹ is independently halogen;

m is 0;

R³ is —OR³¹ or —NR³³R³⁴;

R³¹, R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{e1}, R^{e2} and R^{d15} are independently C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring;

G is N;

n is 0 or 1;

R⁴ is independently C₁₋₆ alkyl, or C₁₋₆ alkyl substituted with one or more R⁴⁻¹;

R⁴⁻¹ is independently cyano;

R² is —C(═O)—C(R^{a})═C(R^{b})(R^{f});

R^{a} is independently hydrogen or halogen;

R^{b} and R^{f} are independently hydrogen or C₁₋₆ alkyl.

In a certain embodiment:

the oxygen-containing heterocyclic compound represented by formula I has a structure as follows:

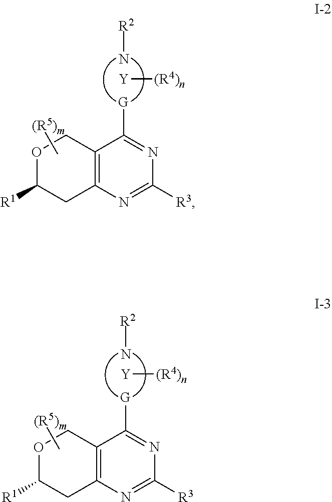

or “a mixture of

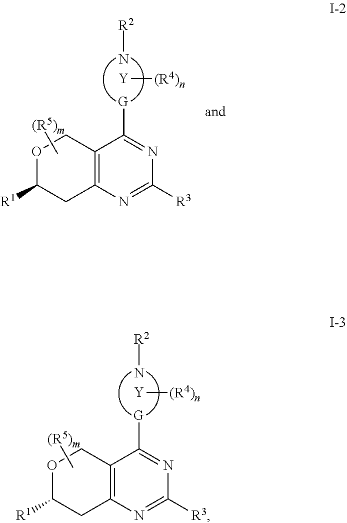

with a molar ratio of, for example, 1:1”.

In a certain embodiment: the oxygen-containing heterocyclic compound represented by formula I has a structure as follows:

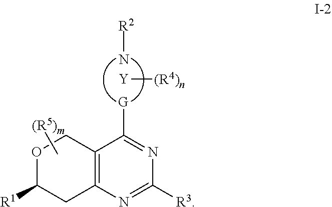

In a certain embodiment:

when R¹ is C₆₋₂₀ aryl, then the C₆₋₂₀ aryl may be phenyl or naphthyl, or may be phenyl or 1-naphthyl.

In a certain embodiment:

when R¹ is “5-12 membered heteroaryl containing 1-4 heteroatoms selected from one or more of O, S and N”, then the “5-12 membered heteroaryl containing 1-4 heteroatoms selected from one or more of O, S and N” may be “9-10 membered heteroaryl containing 1-4 heteroatoms selected from one or more of O, S and N”, or may be “9-10 membered heteroaryl containing 1 heteroatom selected from one of O, S and N”, or may be isoquinolyl, or may be

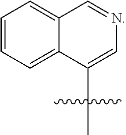 [SMILES: *c1cncc2ccccc12]

In a certain embodiment:

when R¹ is C₆₋₂₀ aryl substituted with one or more R¹⁻⁶, then the C₆₋₂₀ aryl may be phenyl or naphthyl, or may be phenyl or 1-naphthyl.

In a certain embodiment:

when R¹ is C₆₋₂₀ aryl substituted with one or more R¹⁻⁶, then the more R¹⁻⁶ may be two or three R¹⁻⁶.

In a certain embodiment:

when R¹⁻⁶ is independently halogen, then the halogen may be fluorine, chlorine, bromine or iodine, or may be fluorine or chlorine.

In a certain embodiment:

when R¹⁻⁶ is independently C₁₋₆ alkyl, then the C₁₋₄ alkyl may be C₁₋₄ alkyl, or may be methyl, ethyl, n-propyl, isopropyl, n-butyl, isobutyl, sec-butyl or tert-butyl, or may be methyl.

In a certain embodiment:

when R¹⁻⁶ is independently C₁₋₆ alkyl substituted with one or more R¹⁻⁶⁻¹, then the C₁₋₆ alkyl may be C₁₋₄ alkyl, or may be methyl, ethyl, n-propyl, isopropyl, n-butyl, isobutyl, sec-butyl or tert-butyl, or may be methyl.

In a certain embodiment;

when R¹⁻⁶ is independently C₁₋₆ alkyl substituted with one or more R¹⁻⁶⁻¹, then the more R¹⁻⁶⁻¹ may be two or three R¹⁻⁶⁻¹.

In a certain embodiment:

when R¹⁻⁶⁻¹ is independently halogen, then the halogen may be fluorine, chlorine, bromine or iodine, or may be fluorine.

In a certain embodiment:

when R¹⁻⁶ is independently C₁₋₆ alkyl substituted with one or more R¹⁴⁻¹, then the C₁₋₆ alkyl substituted with one or more R¹⁻⁶⁻¹ may be trifluoromethyl.

In a certain embodiment:

when R¹ is C₆₋₂₀ aryl substituted with one or more R¹⁻⁶, then the C₆₋₂₀ aryl substituted with one or more R¹⁻⁶ is

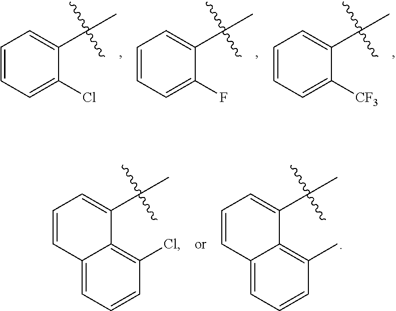 [SMILES: *c1ccccc1Cl ; *c1ccccc1F ; *c1ccccc1C(F)(F)F ; *c1cccc2cccc(Cl)c12 ; *c1cccc2cccc(C)c12]

In a certain embodiment:

when R¹ is C₆₋₂₀ aryl substituted with one or more R¹⁻⁶, then the C₆₋₂₀ aryl substituted with one or more R¹⁻⁶ is

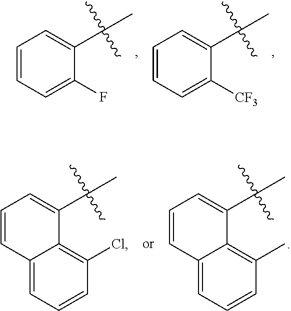 [SMILES: *c1ccccc1F ; *c1ccccc1C(F)(F)F ; *c1cccc2cccc(Cl)c12 ; *c1cccc2cccc(C)c12]

In a certain embodiment:

when R³³ is independently C₁₋₆ alkyl, then the C₁₋₆ alkyl may be C₁₋₄ alkyl, or may be methyl, ethyl, n-propyl, isopropyl, n-butyl, isobutyl, sec-butyl or tert-butyl, or may be methyl, ethyl, n-propyl or isopropyl.

In a certain embodiment:

when R³¹, R³³ and R³⁴ are independently C₁₋₆ alkyl substituted with one or more R³¹⁻¹, then the C₁₋₆ alkyl may be C₁₋₄ alkyl, or may be methyl, ethyl, n-propyl, isopropyl, n-butyl, isobutyl, sec-butyl or tert-butyl, or may be methyl, ethyl, n-propyl or isopropyl.

In a certain embodiment:

when R³¹, R³³ and R³¹ are independently C₁₋₆ alkyl substituted with one or more R³¹⁻¹, then the more R³¹⁻¹ may be two or three R³¹⁻¹.

In a certain embodiment:

when R³¹⁻¹ is independently “4-10 membered heterocycloalkyl containing 1-3 heteroatoms selected from one or more of O and N” substituted with one or more R^{d15}, then the “4-10 membered heterocycloalkyl containing 1-3 heteroatoms selected from one or more of O and N” may be “5-7 membered heterocycloalkyl containing 1-3 heteroatoms selected from one or more of O and N”, or may be “5-7 membered heterocycloalkyl containing 1 heteroatom selected from one of O and N”, or may be tetrahydropyrrolyl, or more particularly, tetrahydropyrrole-2-yl.

In a certain embodiment:

when R³¹⁻¹ is independently “4-10 membered heterocycloalkyl containing 1-3 heteroatoms selected from one or more of O and N” substituted with one or more R^{d15}, then the more R^{d15} may be two or three R^{d15}.

In a certain embodiment:

when R^{d15} is independently C₁₋₆ alkyl, then the C₁₋₆ alkyl may be C₁₋₄ alkyl, or may be methyl, ethyl, n-propyl, isopropyl, n-butyl, isobutyl, sec-butyl or tert-butyl, or may be methyl.

In a certain embodiment:

when R^{d15} is independently halogen, then the halogen may be fluorine, chlorine, bromine or iodine, or may be fluorine.

In a certain embodiment:

when R^{e1} and R^{e2} are independently C₁₋₆ alkyl, then the C₁₋₆ alkyl may be C₁₋₄ alkyl, or may be methyl, ethyl, n-propyl, isopropyl, n-butyl, isobutyl, sec-butyl or tert-butyl, or may be methyl or ethyl.

In a certain embodiment:

when R³¹, R³³ and R³⁴ are independently C₁₋₆ alkyl substituted with one or more R³¹⁻¹, then the C₁₋₆ alkyl substituted with one or more R³¹⁻¹ may be

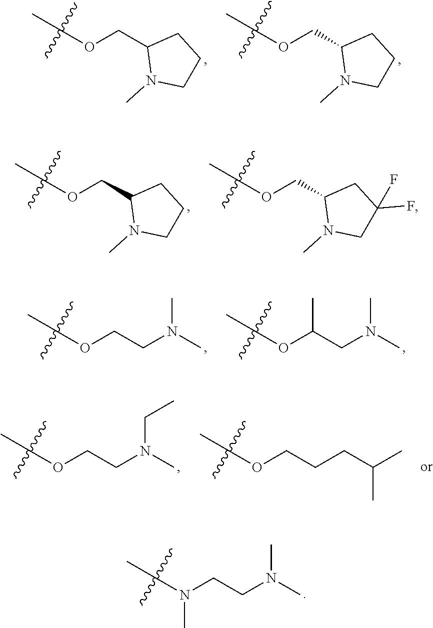

In a certain embodiment:

when ring Y is a 4-12 membered heterocyclic ring containing 1-4 N atoms, then the 4-12 membered heterocyclic ring containing 1-4 N atoms may be a 6-9 membered heterocyclic ring containing 1-2 N atoms or may be

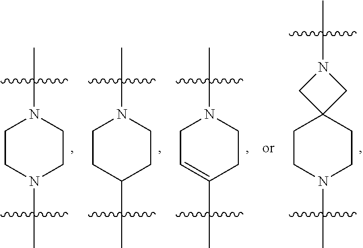

which, at its upper end, is connected to R².

In a certain embodiment:

the oxygen-containing heterocyclic compound represented by formula I has a structure as follows:

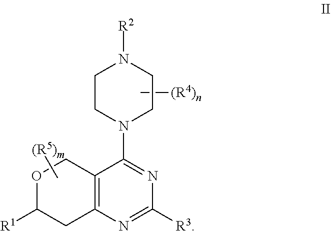

In a certain embodiment:

when R⁴ is independently C₁₋₆ alkyl, then the C₁₋₆ alkyl may be C₁₋₄ alkyl, or may be methyl, ethyl, n-propyl, isopropyl, n-butyl, isobutyl, sec-butyl or tert-butyl, or may be methyl.

In a certain embodiment:

when R⁴ is independently C₁₋₆ alkyl substituted with one or more R⁴⁻¹, then the C₁₋₆ alkyl may be C₁₋₄ alkyl, or may be methyl, ethyl, n-propyl, isopropyl, n-butyl, isobutyl, sec-butyl or tert-butyl, or may be methyl.

In a certain embodiment:

when R⁴ is independently C₁₋₆ alkyl substituted with one or more R⁴⁻¹, then the more R⁴⁻¹ may be two or three R⁴⁻¹.

In a certain embodiment:

when R⁴ is independently C₁₋₆ alkyl substituted with one or more R⁴⁻¹, then the C₁₋₆ alkyl substituted with one or more R⁴⁻¹ may be hydroxymethyl, cyanomethyl or

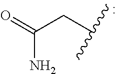 [SMILES: *CC(N)=O]

for example, cyanomethyl or

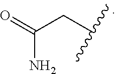 [SMILES: *CC(N)=O]

In a certain embodiment:

when R⁴ is independently C₁₋₆ alkyl substituted with one or more R⁴⁻¹, then the C₁₋₆ alkyl substituted with one or more R⁴¹ may be

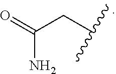 [SMILES: *CC(N)=O]

In a certain embodiment:

when R⁴ is independently C₁₋₆ alkyl substituted with one or more R⁴⁻¹, then the C₁₋₆ alkyl substituted with one or more R⁴⁻¹ may be hydroxymethyl or cyanomethyl.

In a certain embodiment:

when R⁴ is independently C₁₋₆ alkyl substituted with one or more R⁴⁻¹, then the C₁₋₆ alkyl substituted with one or more R⁴⁻¹ may be hydroxymethyl.

In a certain embodiment:

when R⁴ is independently C₁₋₆ alkyl substituted with one or more R⁴⁻¹, then the C₁₋₆ alkyl substituted with one or more R⁴⁻¹ may be cyanomethyl.

In a certain embodiment:

when R^{a} is independently halogen, then the halogen may be fluorine, chlorine, bromine or iodine, or may be fluorine.

In a certain embodiment:

when R^{b} and R^{f} are independently C₁₋₆ alkyl, then the C₁₋₆ alkyl may be C₁₋₄ alkyl, or may be methyl, ethyl, n-propyl, isopropyl, n-butyl, isobutyl, sec-butyl or tert-butyl, or may be methyl.

In a certain embodiment:

when R^{b} and R^{f} are independently C₁₋₆ alkyl-C(═O)—, then the C₁₋₆ alkyl in the C₁₋₆ alkyl-C(═O)— may be C₁₋₄ alkyl, or may be methyl, ethyl, n-propyl, isopropyl, n-butyl, isobutyl, sec-butyl or tert-butyl, or may be methyl.

In a certain embodiment:

when R^{b} and R^{f} are independently C₁₋₆ alkyl substituted with one or more R^{b-1}, then the C₁₋₆ alkyl may be C₁₋₄ alkyl, or may be methyl, ethyl, n-propyl, isopropyl, n-butyl, isobutyl, sec-butyl or tert-butyl, or may be methyl.

In a certain embodiment:

when R^{b} and R^{f} are independently C₁₋₆ alkyl substituted with one or more R^{b-1}, then the more R^{b-1} may be two or three R^{b-1}.

In a certain embodiment:

when R^{10j} and R^{10k} are independently C₁₋₆ alkyl, then the C₁₋₆ alkyl may be C₁₋₄ or may be methyl, ethyl, n-propyl, isopropyl, n-butyl, isobutyl, sec-butyl or tert-butyl, or may be methyl.

In a certain embodiment:

when R^{10j} and R^{10k} taken together with the N atom to which they are attached form “4-10 membered heterocycloalkyl containing 1-3 heteroatoms selected from one or more of 0 and N”, then the “4-10 membered heterocycloalkyl containing 1-3 heteroatoms selected from one or more of O and N” may be “5-6 membered heterocycloalkyl containing 2 heteroatoms selected from 0 and N”, or may be

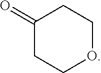

In a certain embodiment:

the R² may be CN.

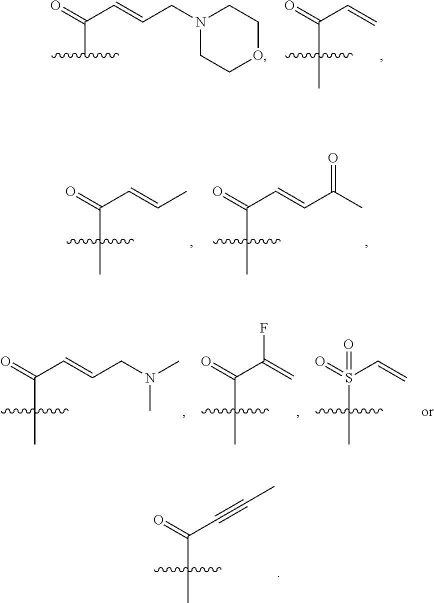

In a certain embodiment:

the R² may be CN.

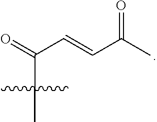 [SMILES: *C(=O)/C=C/C(C)=O]

In a certain embodiment:

the R² may be

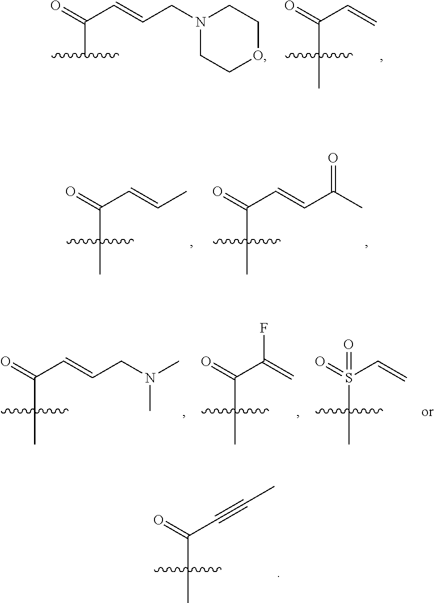

In a certain embodiment:

the R² may be

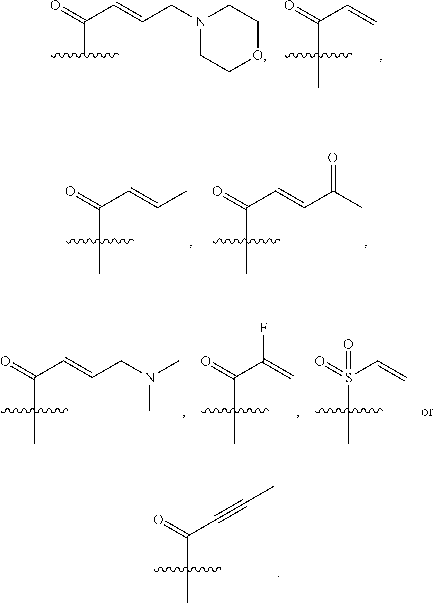

In a certain embodiment, the oxygen-containing heterocyclic compound represented by formula I has any one of the following structures:

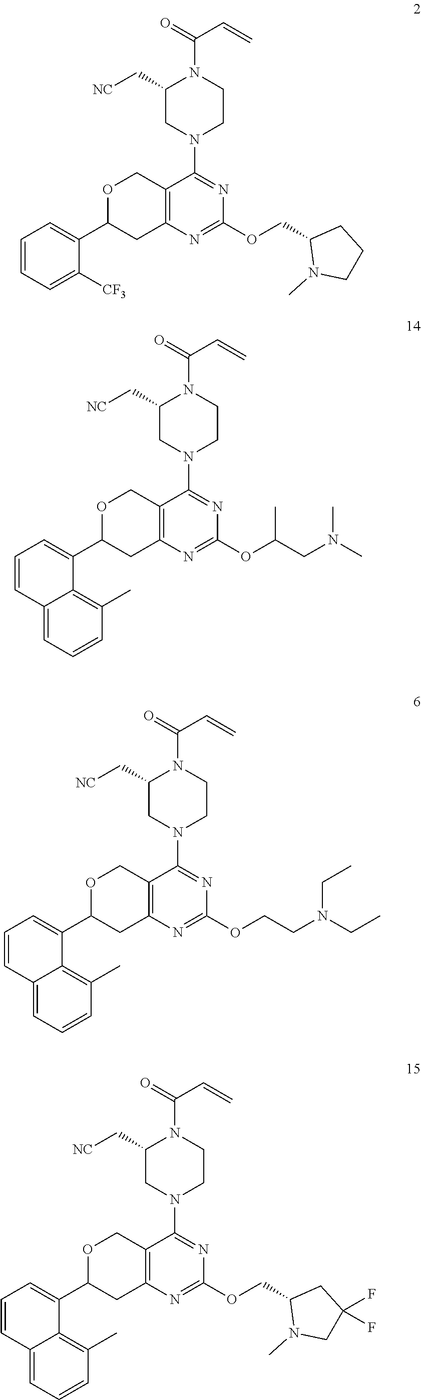 [SMILES: C=CC(=O)N1CCN(c2nc(OC[C@@H]3CCCN3C)nc3c2COC(c2ccccc2C(F)(F)F)C3)C[C@@H]1CC#N ; C=CC(=O)N1CCN(c2nc(OC(C)CN(C)C)nc3c2COC(c2cccc4cccc(C)c24)C3)C[C@@H]1CC#N ; C=CC(=O)N1CCN(c2nc(OCCN(CC)CC)nc3c2COC(c2cccc4cccc(C)c24)C3)C[C@@H]1CC#N ; C=CC(=O)N1CCN(c2nc(OC[C@@H]3CC(F)(F)CN3C)nc3c2COC(c2cccc4cccc(C)c24)C3)C[C@@H]1CC#N]

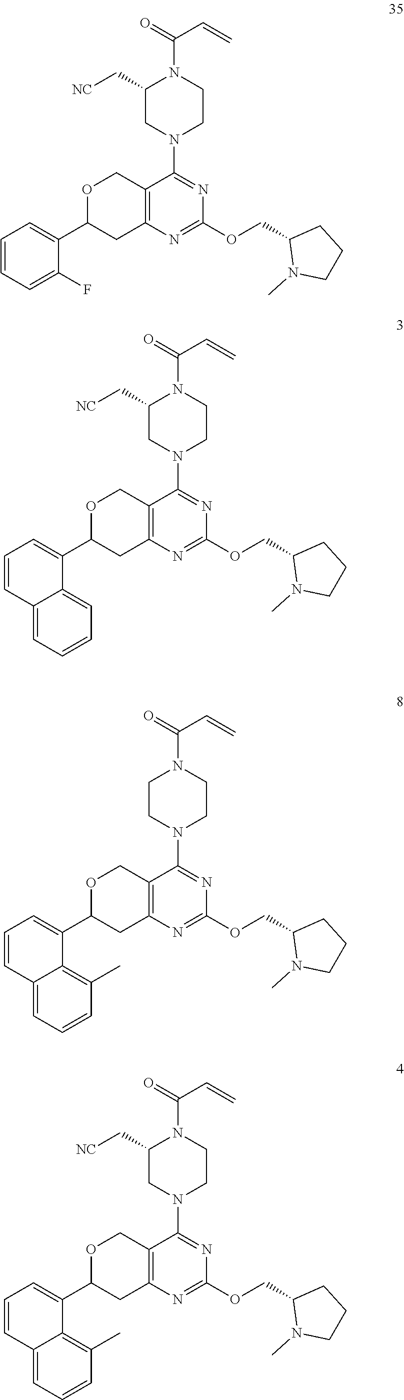 [SMILES: C=CC(=O)N1CCN(c2nc(OC[C@@H]3CCCN3C)nc3c2COC(c2ccccc2F)C3)C[C@@H]1CC#N ; C=CC(=O)N1CCN(c2nc(OC[C@@H]3CCCN3C)nc3c2COC(c2cccc4ccccc24)C3)C[C@@H]1CC#N ; C=CC(=O)N1CCN(c2nc(OC[C@@H]3CCCN3C)nc3c2COC(c2cccc4cccc(C)c24)C3)CC1 ; C=CC(=O)N1CCN(c2nc(OC[C@@H]3CCCN3C)nc3c2COC(c2cccc4cccc(C)c24)C3)C[C@@H]1CC#N]

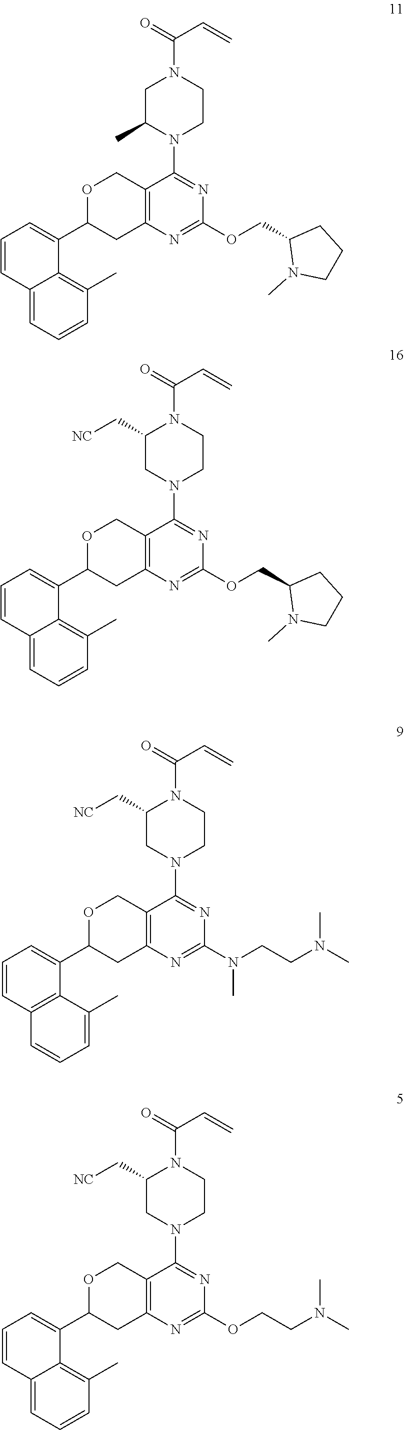 [SMILES: C=CC(=O)N1CCN(c2nc(OC[C@@H]3CCCN3C)nc3c2COC(c2cccc4cccc(C)c24)C3)[C@@H](C)C1 ; C=CC(=O)N1CCN(c2nc(OC[C@H]3CCCN3C)nc3c2COC(c2cccc4cccc(C)c24)C3)C[C@@H]1CC#N ; C=CC(=O)N1CCN(c2nc(N(C)CCN(C)C)nc3c2COC(c2cccc4cccc(C)c24)C3)C[C@@H]1CC#N ; C=CC(=O)N1CCN(c2nc(OCCN(C)C)nc3c2COC(c2cccc4cccc(C)c24)C3)C[C@@H]1CC#N]

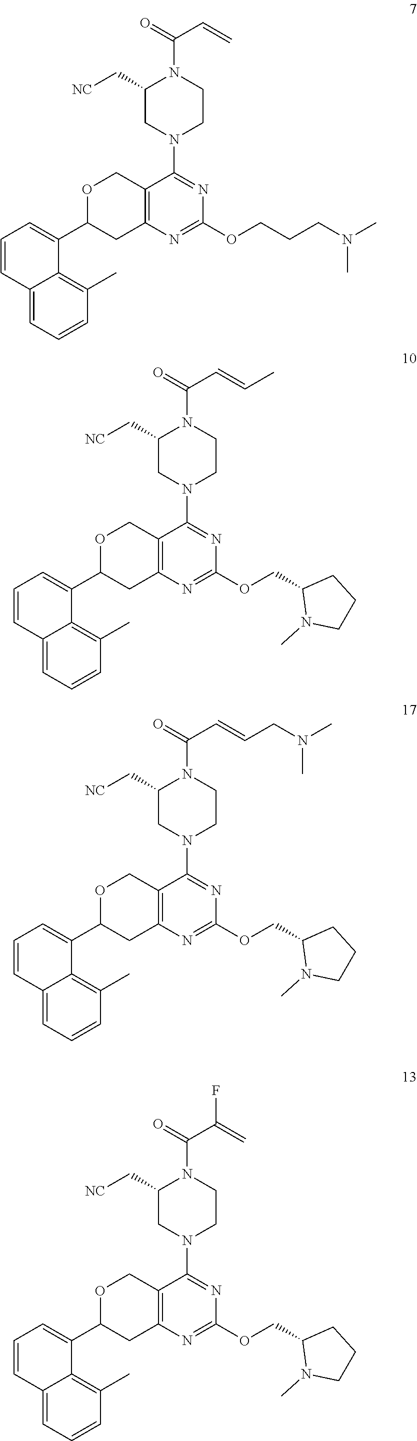 [SMILES: C=CC(=O)N1CCN(c2nc(OCCCN(C)C)nc3c2COC(c2cccc4cccc(C)c24)C3)C[C@@H]1CC#N ; C/C=C/C(=O)N1CCN(c2nc(OC[C@@H]3CCCN3C)nc3c2COC(c2cccc4cccc(C)c24)C3)C[C@@H]1CC#N ; Cc1cccc2cccc(C3Cc4nc(OC[C@@H]5CCCN5C)nc(N5CCN(C(=O)/C=C/CN(C)C)[C@@H](CC#N)C5)c4CO3)c12 ; C=C(F)C(=O)N1CCN(c2nc(OC[C@@H]3CCCN3C)nc3c2COC(c2cccc4cccc(C)c24)C3)C[C@@H]1CC#N]

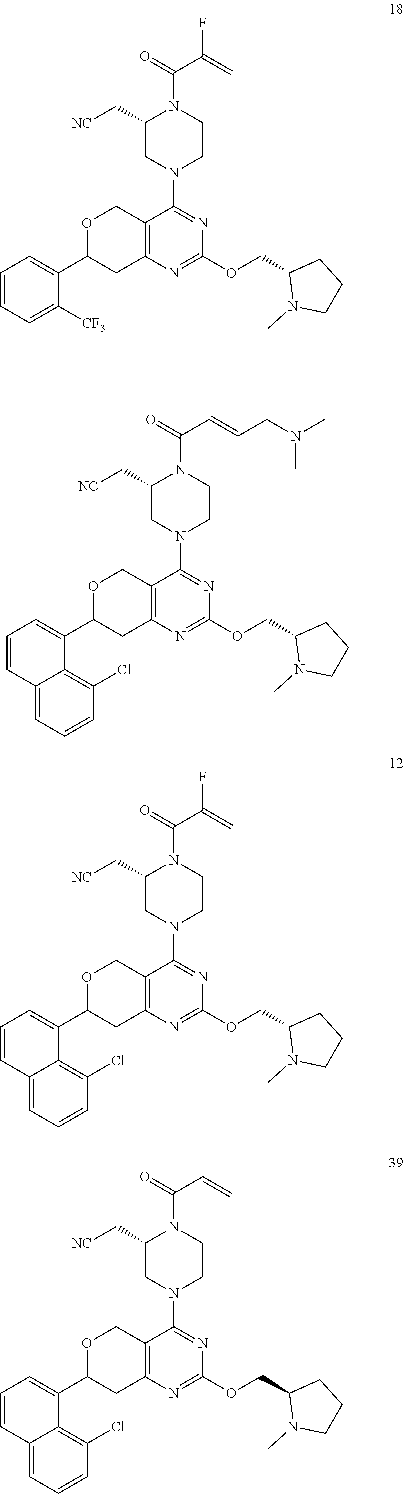 [SMILES: C=C(F)C(=O)N1CCN(c2nc(OC[C@@H]3CCCN3C)nc3c2COC(c2ccccc2C(F)(F)F)C3)C[C@@H]1CC#N ; CN(C)C/C=C/C(=O)N1CCN(c2nc(OC[C@@H]3CCCN3C)nc3c2COC(c2cccc4cccc(Cl)c24)C3)C[C@@H]1CC#N ; C=C(F)C(=O)N1CCN(c2nc(OC[C@@H]3CCCN3C)nc3c2COC(c2cccc4cccc(Cl)c24)C3)C[C@@H]1CC#N ; C=CC(=O)N1CCN(c2nc(OC[C@H]3CCCN3C)nc3c2COC(c2cccc4cccc(Cl)c24)C3)C[C@@H]1CC#N]

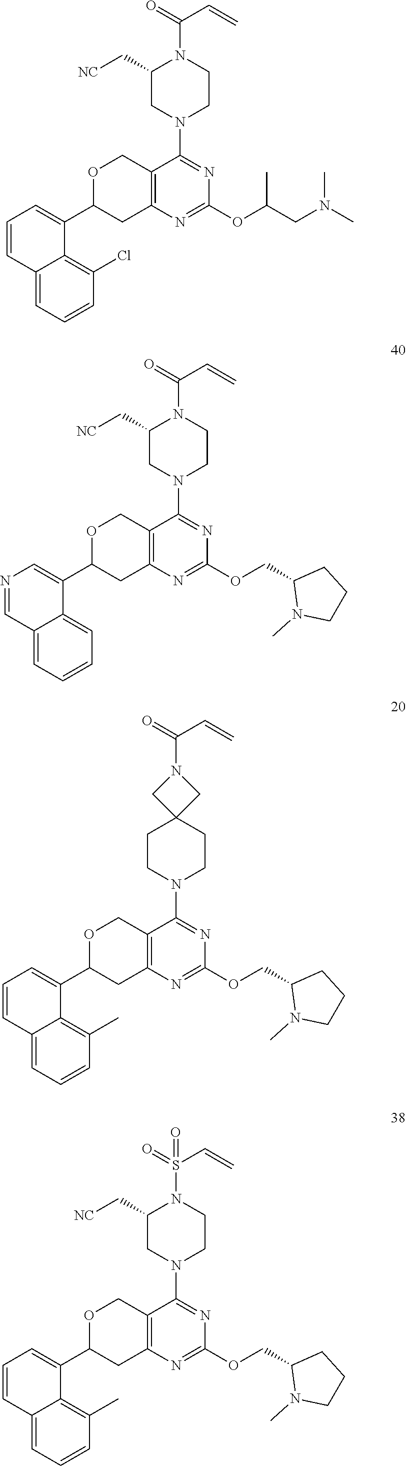 [SMILES: C=CC(=O)N1CCN(c2nc(OC(C)CN(C)C)nc3c2COC(c2cccc4cccc(Cl)c24)C3)C[C@@H]1CC#N ; C=CC(=O)N1CCN(c2nc(OC[C@@H]3CCCN3C)nc3c2COC(c2cncc4ccccc24)C3)C[C@@H]1CC#N ; C=CC(=O)N1CC2(CCN(c3nc(OC[C@@H]4CCCN4C)nc4c3COC(c3cccc5cccc(C)c35)C4)CC2)C1 ; C=CS(=O)(=O)N1CCN(c2nc(OC[C@@H]3CCCN3C)nc3c2COC(c2cccc4cccc(C)c24)C3)C[C@@H]1CC#N]

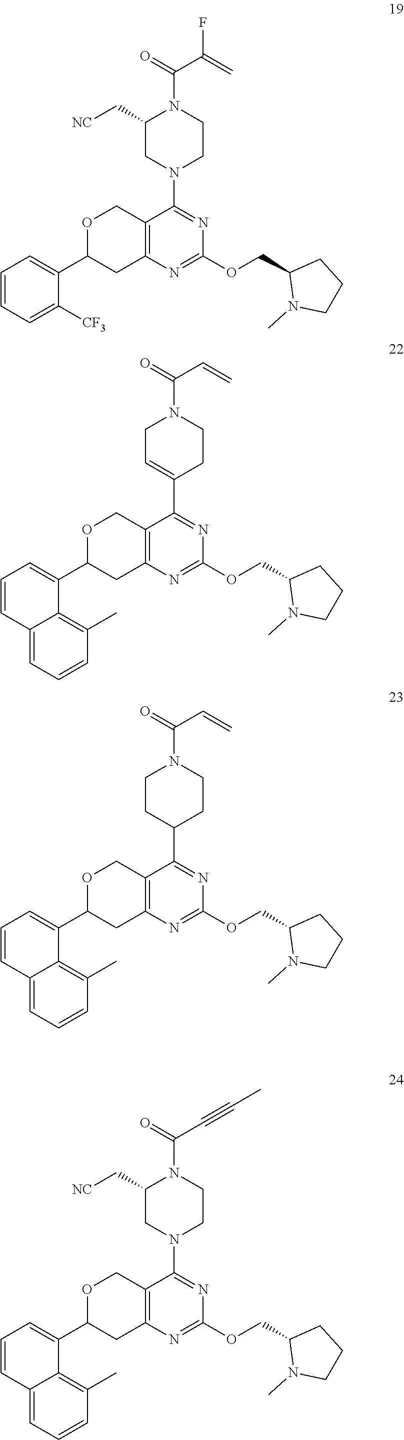 [SMILES: C=C(F)C(=O)N1CCN(c2nc(OC[C@H]3CCCN3C)nc3c2COC(c2ccccc2C(F)(F)F)C3)C[C@@H]1CC#N ; C=CC(=O)N1CC=C(c2nc(OC[C@@H]3CCCN3C)nc3c2COC(c2cccc4cccc(C)c24)C3)CC1 ; C=CC(=O)N1CCC(c2nc(OC[C@@H]3CCCN3C)nc3c2COC(c2cccc4cccc(C)c24)C3)CC1 ; CC#CC(=O)N1CCN(c2nc(OC[C@@H]3CCCN3C)nc3c2COC(c2cccc4cccc(C)c24)C3)C[C@@H]1CC#N]

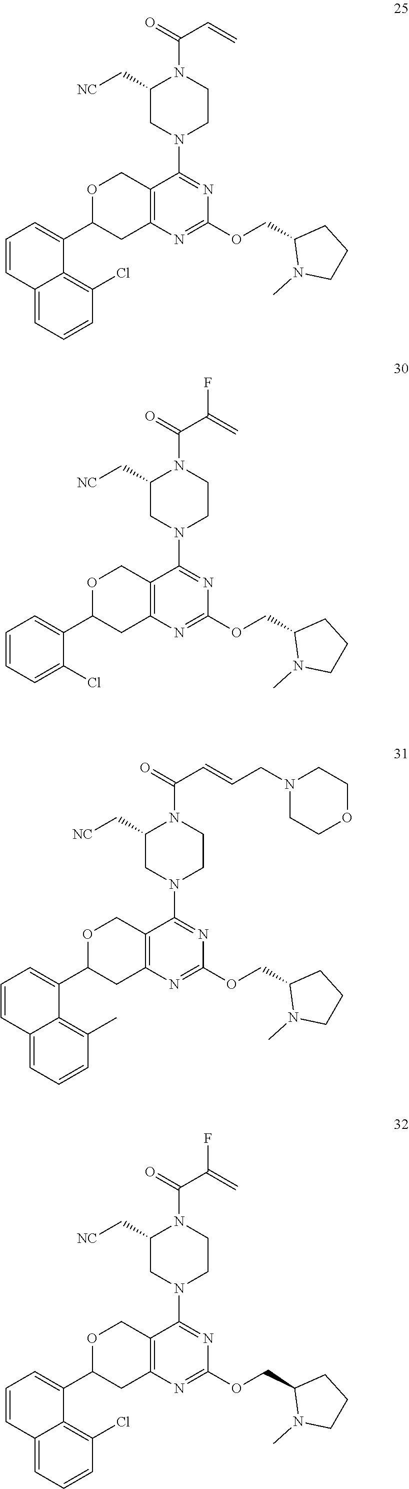 [SMILES: C=CC(=O)N1CCN(c2nc(OC[C@@H]3CCCN3C)nc3c2COC(c2cccc4cccc(Cl)c24)C3)C[C@@H]1CC#N ; C=C(F)C(=O)N1CCN(c2nc(OC[C@@H]3CCCN3C)nc3c2COC(c2ccccc2Cl)C3)C[C@@H]1CC#N ; Cc1cccc2cccc(C3Cc4nc(OC[C@@H]5CCCN5C)nc(N5CCN(C(=O)/C=C/CN6CCOCC6)[C@@H](CC#N)C5)c4CO3)c12 ; C=C(F)C(=O)N1CCN(c2nc(OC[C@H]3CCCN3C)nc3c2COC(c2cccc4cccc(Cl)c24)C3)C[C@@H]1CC#N]

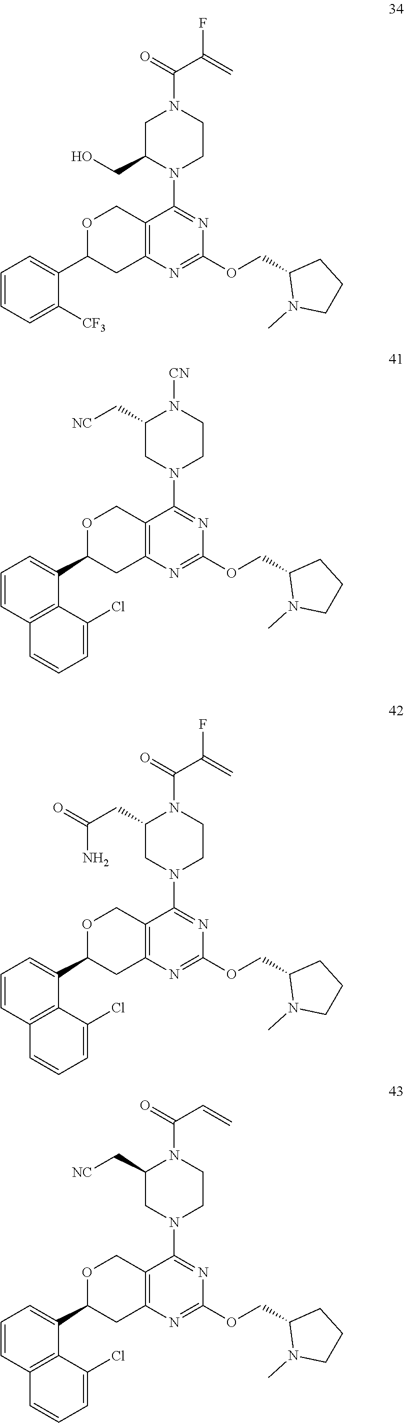 [SMILES: C=C(F)C(=O)N1CCN(c2nc(OC[C@@H]3CCCN3C)nc3c2COC(c2ccccc2C(F)(F)F)C3)[C@@H](CO)C1 ; CN1CCC[C@H]1COc1nc2c(c(N3CCN(C#N)[C@@H](CC#N)C3)n1)CO[C@H](c1cccc3cccc(Cl)c13)C2 ; C=C(F)C(=O)N1CCN(c2nc(OC[C@@H]3CCCN3C)nc3c2CO[C@H](c2cccc4cccc(Cl)c24)C3)C[C@@H]1CC(N)=O ; C=CC(=O)N1CCN(c2nc(OC[C@@H]3CCCN3C)nc3c2CO[C@H](c2cccc4cccc(Cl)c24)C3)C[C@H]1CC#N]

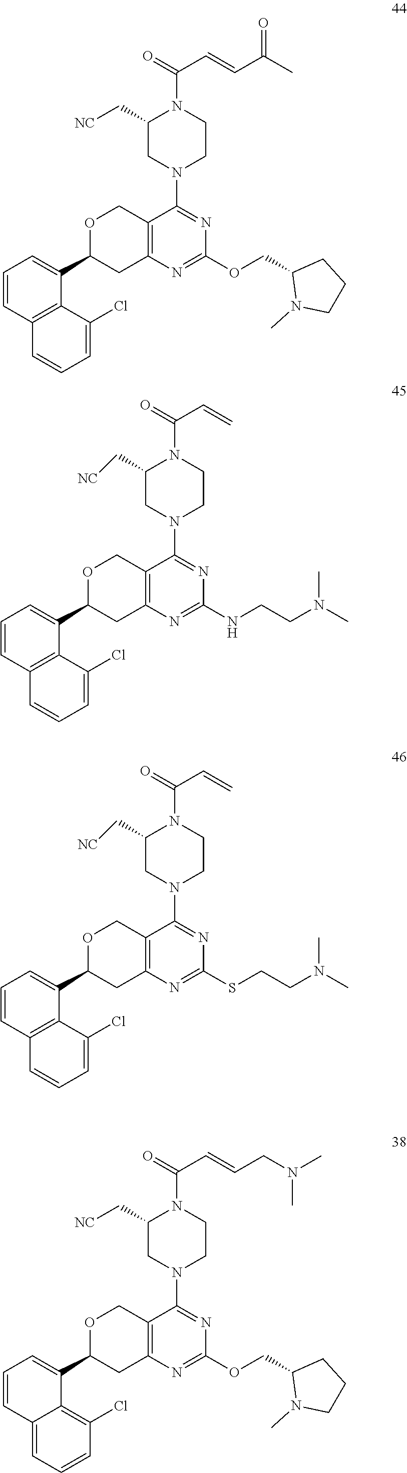 [SMILES: CC(=O)/C=C/C(=O)N1CCN(c2nc(OC[C@@H]3CCCN3C)nc3c2CO[C@H](c2cccc4cccc(Cl)c24)C3)C[C@@H]1CC#N ; C=CC(=O)N1CCN(c2nc(NCCN(C)C)nc3c2CO[C@H](c2cccc4cccc(Cl)c24)C3)C[C@@H]1CC#N ; C=CC(=O)N1CCN(c2nc(SCCN(C)C)nc3c2CO[C@H](c2cccc4cccc(Cl)c24)C3)C[C@@H]1CC#N ; CN(C)C/C=C/C(=O)N1CCN(c2nc(OC[C@@H]3CCCN3C)nc3c2CO[C@H](c2cccc4cccc(Cl)c24)C3)C[C@@H]1CC#N]

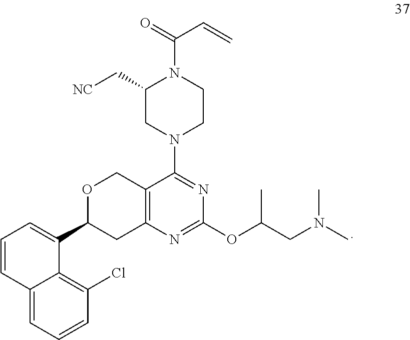 [SMILES: C=CC(=O)N1CCN(c2nc(OC(C)CN(C)C)nc3c2CO[C@H](c2cccc4cccc(Cl)c24)C3)C[C@@H]1CC#N]

In a certain embodiment, the oxygen-containing heterocyclic compound represented by formula I is any one of the following compounds:

compound

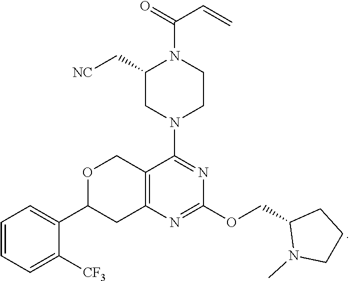 [SMILES: C=CC(=O)N1CCN(c2nc(OC[C@@H]3CCCN3C)nc3c2COC(c2ccccc2C(F)(F)F)C3)C[C@@H]1CC#N]

which has a retention time of 0.92 min under the following conditions: equipment: SFC Method Station (Thar. Waters); chromatographic column: OD-H 4.6*100 mm, 5 μm (Daicel): column temperature: 40° C.: mobile phase; CO₂/MeOH (0.1% TEA)=65/35; flow rate: 4.0 ml/min: wavelength: 254 nm; back pressure: 120 bar;

compound

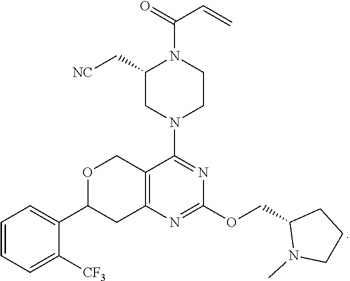 [SMILES: C=CC(=O)N1CCN(c2nc(OC[C@@H]3CCCN3C)nc3c2COC(c2ccccc2C(F)(F)F)C3)C[C@@H]1CC#N]

which has a retention time of 2.74 min wider the following conditions: equipment: SFC Method Station (Thar. Waters): chromatographic column: OD-H 4.6*100 mm, 5 μm (Daicel): column temperature: 40° C.: mobile phase; CO₂/MeOH (0.1% TEA)=65135: flow rate: 4.0 ml/min: wavelength: 254 nm: back pressure; 120 bar;

compound

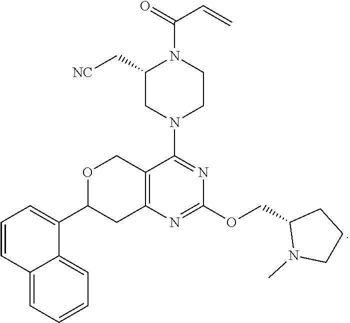 [SMILES: C=CC(=O)N1CCN(c2nc(OC[C@@H]3CCCN3C)nc3c2COC(c2cccc4ccccc24)C3)C[C@@H]1CC#N]

which has a retention time of 0.97 min under the following conditions: equipment: SFC Method Station (Thar. Waters): chromatographic column: AD-H 4.6*100 mm, 5 μm (Daicel): column temperature: 40° C.: mobile phase; CO₂/ETOH (0.5% TEA)=55.45: flow rate: 4.0 ml/min: wavelength: 254 nm: back pressure; 120 bar;

compound

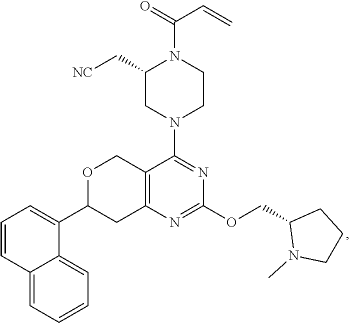 [SMILES: C=CC(=O)N1CCN(c2nc(OC[C@@H]3CCCN3C)nc3c2COC(c2cccc4ccccc24)C3)C[C@@H]1CC#N]

which has a retention tune of 2.40 min under the following conditions: equipment: SFC Method Station (Thar. Waters): chromatographic AD-H 4.6*100 mm, 5 μm (Daicel): column temperature: 40° C.; mobile phase; CO₂/ETOH (0.5% TEA)=55/45: flow rate: 4.0 ml/min: wavelength: 254 nm: back pressure; 120 bar;

compound

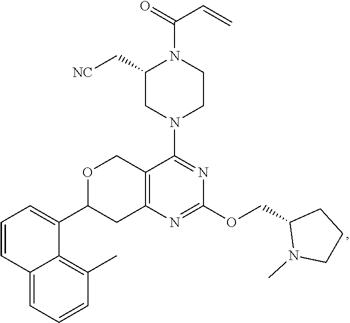 [SMILES: C=CC(=O)N1CCN(c2nc(OC[C@@H]3CCCN3C)nc3c2COC(c2cccc4cccc(C)c24)C3)C[C@@H]1CC#N]

which has a retention time of 0.97 min under the following conditions: equipment: SFC Method Station (Thar. Waters): chromatographic column: OJ-H 4.6*100 mm, 5 μm (Daicel): column temperature: 40° C.: mobile phase; CO₂/Methanol (0.1% TEA)=60/40; flow rate: 4.0 ml/min: wavelength: 254 nm: back pressure: 120 bar;

compound

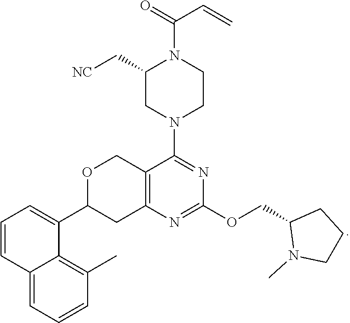 [SMILES: C=CC(=O)N1CCN(c2nc(OC[C@@H]3CCCN3C)nc3c2COC(c2cccc4cccc(C)c24)C3)C[C@@H]1CC#N]

which has a retention time of 1.94 min under the following conditions: equipment: SFC Method Station (Thar, Waters); chromatographic column: OJ-H 4.6*100 mm, 5 μm (Daicel): column temperature: 40° C.; mobile phase; CO₂/Methanol (0.1% TEA)=60:40; flow rate: 4.0 ml/min: wavelength: 254 nm: back pressure: 120 bar;

compound

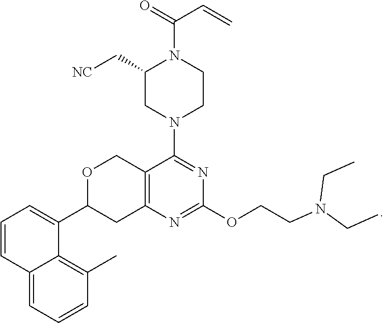 [SMILES: C=CC(=O)N1CCN(c2nc(OCCN(CC)CC)nc3c2COC(c2cccc4cccc(C)c24)C3)C[C@@H]1CC#N]

which has a retention time of 1.22 min under the following conditions: equipment: SFC Method Station (Thar. Waters); chromatographic column: CHIRALCEL 03-H 4.6*100 mm, 5 μm (Daicel): column temperature: 40° C.; mobile phase: CO₂/MeOH (0.1% TEA)=65/35: flow rate: 1.0 ml/min; wavelength: 214 nm; back pressure: 120 bar;

compound

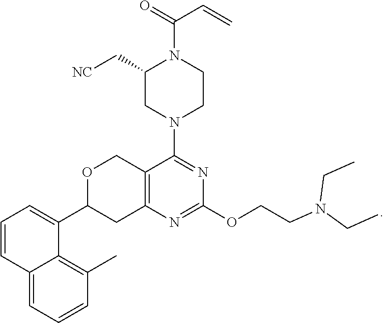 [SMILES: C=CC(=O)N1CCN(c2nc(OCCN(CC)CC)nc3c2COC(c2cccc4cccc(C)c24)C3)C[C@@H]1CC#N]

which has a retention time of 2.67 min under the following conditions: equipment: SFC Method Station (Thar. Waters); chromatographic CHIRALCEL 03-H 4.6*100 mm, 5 μm (Daicel): column temperature: 40° C.; mobile phase: CO₂/MeOH (0.1% TEA)=65/35; flow rate: 1.0 ml/min; wavelength: 214 nm: back pressure: 120 bar;

compound

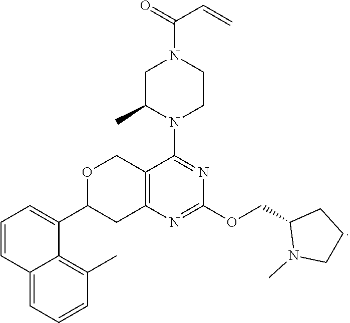 [SMILES: C=CC(=O)N1CCN(c2nc(OC[C@@H]3CCCN3C)nc3c2COC(c2cccc4cccc(C)c24)C3)[C@@H](C)C1]

which has a retention time of 3.26 min under the following conditions: instrument: SFC Method Station (Thar. Waters): chromatographic column: R,R-WHELK-O1 4.6*100 mm, 5 μm (REGIS); column temperature: 40° C.; mobile phase: CO₂/(MeOH/CAN=3:2 (0.1% TEA))=55/45: flow rate: 4.0 ml/min: wavelength; 254 nm: back pressure: 120 bar;

compound

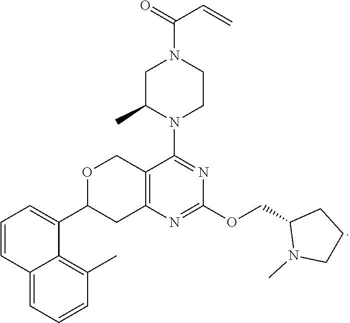 [SMILES: C=CC(=O)N1CCN(c2nc(OC[C@@H]3CCCN3C)nc3c2COC(c2cccc4cccc(C)c24)C3)[C@@H](C)C1]

which has a retention time of 4.16 min under the following conditions: instrument: SFC Method Station (Thar. Waters): chromatographic column: R,R-WHELK-O1 4.6*100 mm, 5 μm (REGIS): column temperature: 40° C.: mobile phase: CO₂/(MeOH/CAN=3:2 (0.1% TEA))=55/45: flow rate: 4.0 ml/min: wavelength; 254 nm: back pressure: 120 bar.

compound

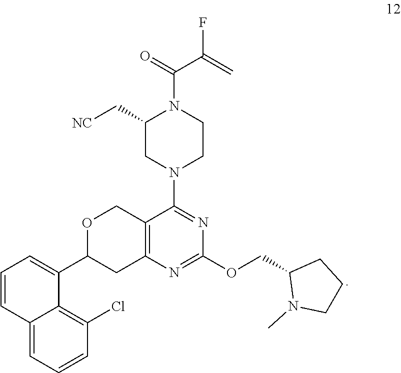 [SMILES: C=C(F)C(=O)N1CCN(c2nc(OC[C@@H]3CCCN3C)nc3c2COC(c2cccc4cccc(Cl)c24)C3)C[C@@H]1CC#N]

which has a retention time of 1.36 min under the following conditions: instrument: SFC Method Station (Thar. Waters): chromatographic column: OJ-H 4.6*100 mm, 5 μm (Daicel); column temperature: 40° C.: mobile phase; CO₂/MeOH (0.1% TEA)=60/40; flow rate: 4.0 ml/min; wavelength: 254 nm; back pressure; 120 bar.

compound

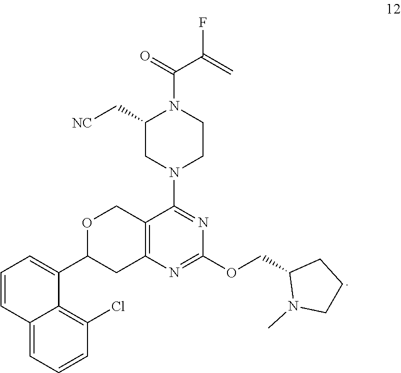 [SMILES: C=C(F)C(=O)N1CCN(c2nc(OC[C@@H]3CCCN3C)nc3c2COC(c2cccc4cccc(Cl)c24)C3)C[C@@H]1CC#N]

which has a retention time of 2.77 renin under the following conditions: instrument: SFC Method Station (Thar. Waters): chromatographic column: OJ-H 4.6*100 mm, 5 μm (Daicel): column temperature: 40° C.: mobile phase; CO₂/MeOH (0.1% TEA)=6040: flow rate: 4.0 ml/min: wavelength: 254 nm: back pressure; 120 bar.

compound

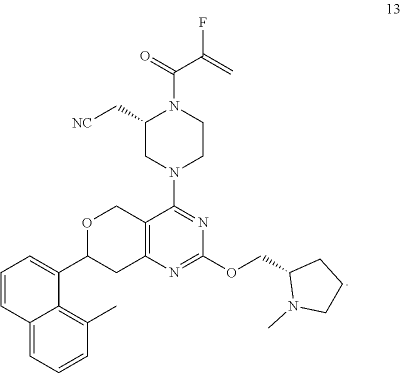 [SMILES: C=C(F)C(=O)N1CCN(c2nc(OC[C@@H]3CCCN3C)nc3c2COC(c2cccc4cccc(C)c24)C3)C[C@@H]1CC#N]

which has a retention time of 1.17 min under the following conditions: instrument: SFC Method Station (Thar. Waters): chromatographic column: OJ-H 4.6*100 mm, 5 μm (Daicel): column temperature: 40° C.; mobile phase; CO₂/MeOH (0.1% TEA)=60/40: flow rate: 4.0 ml min: wavelength: 254 nm: back pressure; 120 bar.

compound

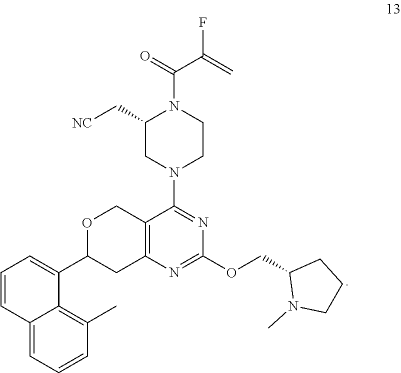 [SMILES: C=C(F)C(=O)N1CCN(c2nc(OC[C@@H]3CCCN3C)nc3c2COC(c2cccc4cccc(C)c24)C3)C[C@@H]1CC#N]

which has a retention time of 2.76 min under the following conditions: instrument: SFC Method Station (Thar. Waters): chromatographic column: OJ-H 4.6*100 mm 5 μm (Daicel): column temperature: 40° C.: mobile phase; CO₂/MeOH (0.1% TEA)=60/40: flow rate: 4.0 wavelength: 254 nm: back pressure; 120 bar.

compound

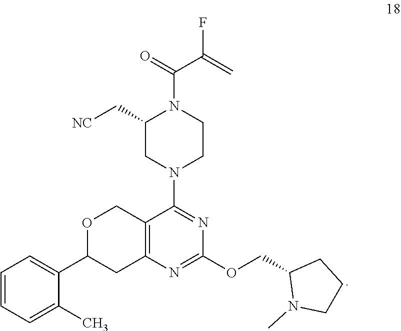 [SMILES: C=C(F)C(=O)N1CCN(c2nc(OC[C@@H]3CCCN3C)nc3c2COC(c2ccccc2C)C3)C[C@@H]1CC#N]

which has a retention time of 0.78 min wider the following conditions: instrument: SFC Method Station (Thar. Waters): chromatographic column: OD-H 4.6*100 mm, 5 μm (Daicel): column temperature: 40° C.; mobile phase; CO₂/MeOH (0.1% TEA)=65:35: flow rate: 4.0 ml/min: wavelength: 254 nm: back pressure; 120 bar.

compound

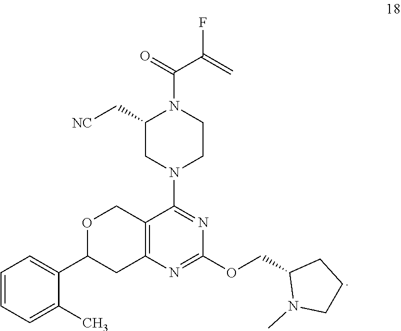 [SMILES: C=C(F)C(=O)N1CCN(c2nc(OC[C@@H]3CCCN3C)nc3c2COC(c2ccccc2C)C3)C[C@@H]1CC#N]

which has a retention time of 2.42 mil under the following conditions: instrument: SFC Method Station (Thar. Waters): chromatographic column: OD-H 4.6*100 mm, 5 μm (Daicel): column temperature: 40° C.; mobile phase; CO₂/MeOH (0.1% TEA)=65/35: flow rate: 4.0 ml/min: wavelength: 254 nm: back pressure; 120 bar.

compound

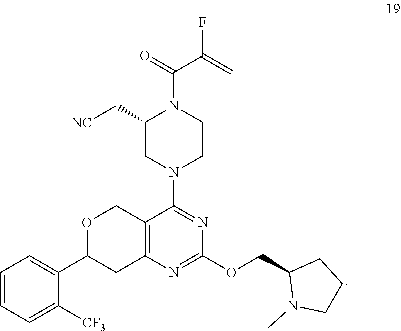 [SMILES: C=C(F)C(=O)N1CCN(c2nc(OC[C@H]3CCCN3C)nc3c2COC(c2ccccc2C(F)(F)F)C3)C[C@@H]1CC#N]

which has a retention time of 0.79 min under the following conditions: instrument: SFC Method Station (Thar. Waters): chromatographic column: OD-H 4.6*100 mm, 5 μm (Daicel): column temperature: 40° C.: mobile phase; CO₂/MeOH (0.1% TEA)=65/35: flow rate: 4.0 ml/min: wavelength: 254 nm: back pressure; 120 bar.

compound

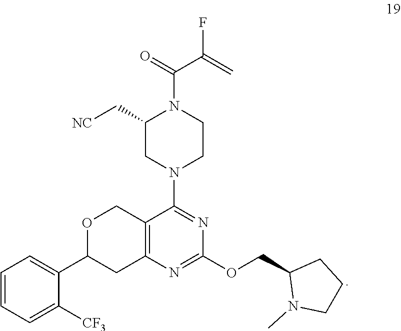 [SMILES: C=C(F)C(=O)N1CCN(c2nc(OC[C@H]3CCCN3C)nc3c2COC(c2ccccc2C(F)(F)F)C3)C[C@@H]1CC#N]

which has a retention tune of 2.29 min under the following conditions: instrument: SFC Method Station (Thar, Waters): chromatographic column: OD-H 4.6*100 mm, 5 μm (Daicel): column temperature: 40° C.; mobile phase; CO₂/MeOH (0.1% TEA)=65/35: flow rate: 4.0 ml/min; wavelength: 254 nm: back pressure; 120 bar.

compound

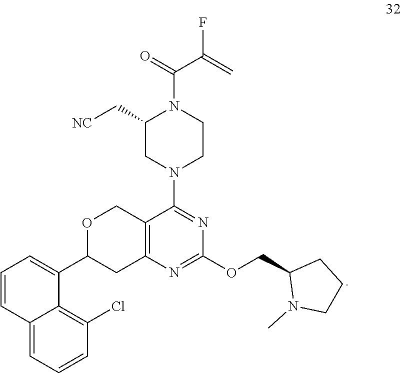 [SMILES: C=C(F)C(=O)N1CCN(c2nc(OC[C@H]3CCCN3C)nc3c2COC(c2cccc4cccc(Cl)c24)C3)C[C@@H]1CC#N]

which has a retention time of 1.45 min under the following conditions: instrument: SEC Method Station (Thar, Waters): chromatographic column: OJ-H 4.6*100 mm, 5 μm (Daicel): column temperature: 40° C.: mobile phase; CO₂/MeOH (0.1% TEA)=60/40; flow rate: 4.0 ml/min: wavelength: 254 nm: back pressure; 120 bar.

compound

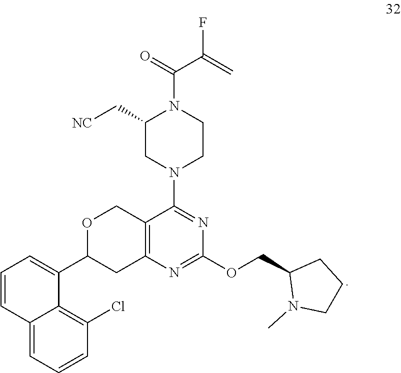 [SMILES: C=C(F)C(=O)N1CCN(c2nc(OC[C@H]3CCCN3C)nc3c2COC(c2cccc4cccc(Cl)c24)C3)C[C@@H]1CC#N]

which has a retention time of 2.81 min under the following conditions: instrument: SFC Method Station (Thar, Waters): chromatographic column: OJ-H 4.6*100 mm, 5 μm (Daicel): column temperature: 40° C.: mobile phase; CO₂/MeOH (0.1% TEA)=60/40: flow rate: 4.0 ml/min: wavelength: 254 nm: back pressure; 120 bar.

In a certain embodiment, the oxygen-containing heterocyclic compound represented by formula I is any one of the following compounds:

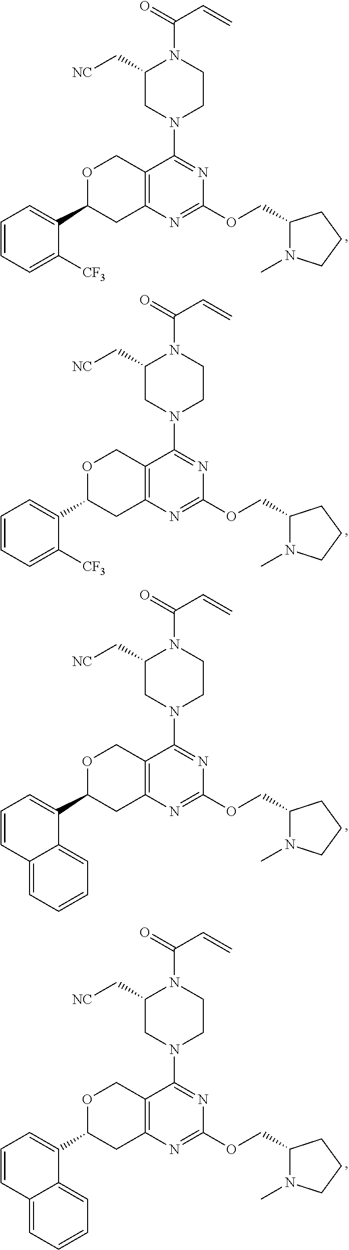 [SMILES: C=CC(=O)N1CCN(c2nc(OC[C@@H]3CCCN3C)nc3c2CO[C@H](c2ccccc2C(F)(F)F)C3)C[C@@H]1CC#N ; C=CC(=O)N1CCN(c2nc(OC[C@@H]3CCCN3C)nc3c2CO[C@@H](c2ccccc2C(F)(F)F)C3)C[C@@H]1CC#N ; C=CC(=O)N1CCN(c2nc(OC[C@@H]3CCCN3C)nc3c2CO[C@H](c2cccc4ccccc24)C3)C[C@@H]1CC#N ; C=CC(=O)N1CCN(c2nc(OC[C@@H]3CCCN3C)nc3c2CO[C@@H](c2cccc4ccccc24)C3)C[C@@H]1CC#N]

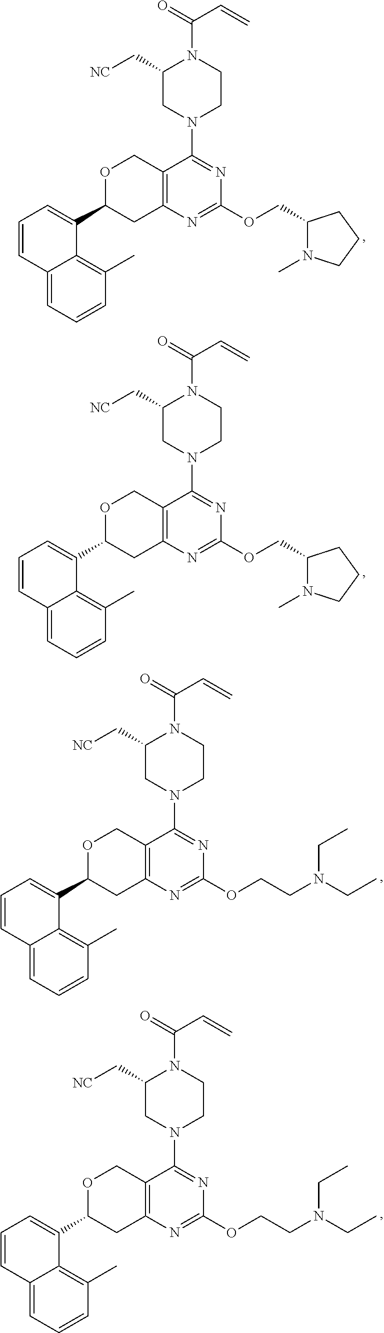 [SMILES: C=CC(=O)N1CCN(c2nc(OC[C@@H]3CCCN3C)nc3c2CO[C@H](c2cccc4cccc(C)c24)C3)C[C@@H]1CC#N ; C=CC(=O)N1CCN(c2nc(OC[C@@H]3CCCN3C)nc3c2CO[C@@H](c2cccc4cccc(C)c24)C3)C[C@@H]1CC#N ; C=CC(=O)N1CCN(c2nc(OCCN(CC)CC)nc3c2CO[C@H](c2cccc4cccc(C)c24)C3)C[C@@H]1CC#N ; C=CC(=O)N1CCN(c2nc(OCCN(CC)CC)nc3c2CO[C@@H](c2cccc4cccc(C)c24)C3)C[C@@H]1CC#N]

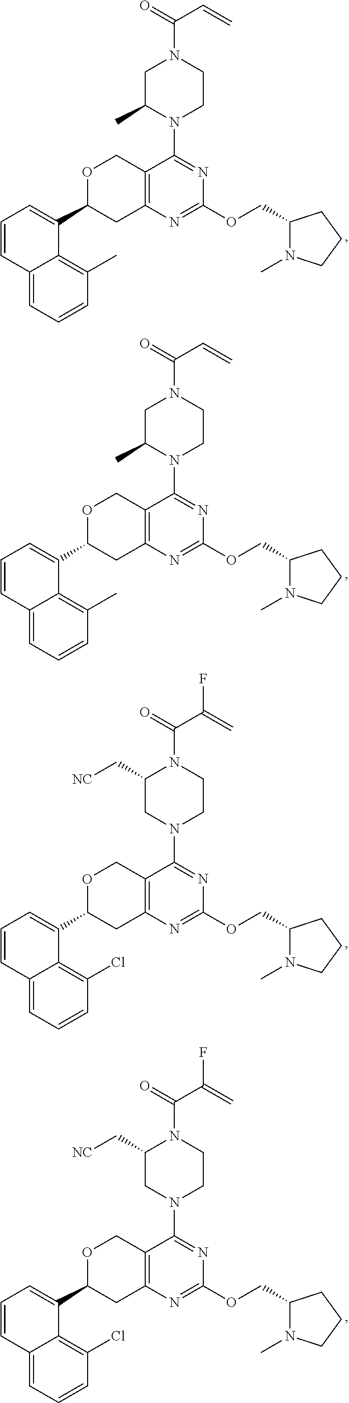 [SMILES: C=CC(=O)N1CCN(c2nc(OC[C@@H]3CCCN3C)nc3c2CO[C@H](c2cccc4cccc(C)c24)C3)[C@@H](C)C1 ; C=CC(=O)N1CCN(c2nc(OC[C@@H]3CCCN3C)nc3c2CO[C@@H](c2cccc4cccc(C)c24)C3)[C@@H](C)C1 ; C=C(F)C(=O)N1CCN(c2nc(OC[C@@H]3CCCN3C)nc3c2CO[C@@H](c2cccc4cccc(Cl)c24)C3)C[C@@H]1CC#N ; C=C(F)C(=O)N1CCN(c2nc(OC[C@@H]3CCCN3C)nc3c2CO[C@H](c2cccc4cccc(Cl)c24)C3)C[C@@H]1CC#N]

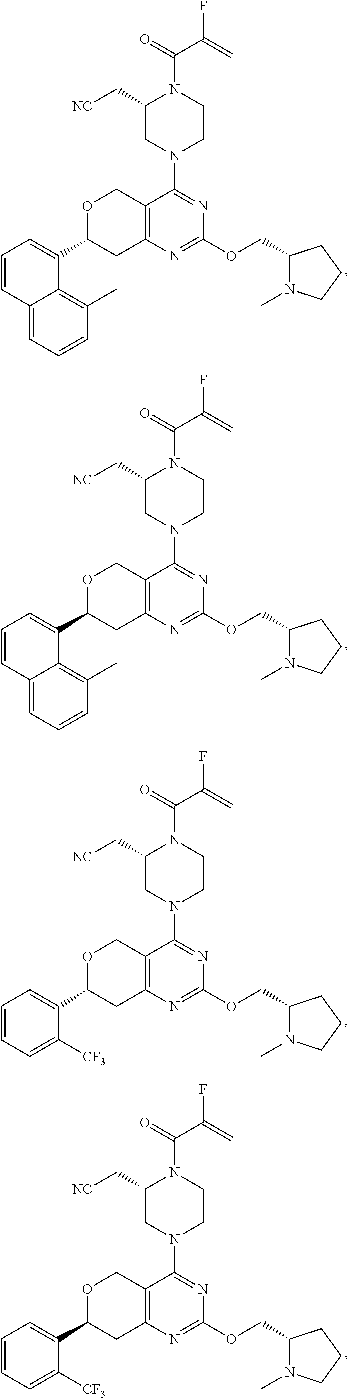 [SMILES: C=C(F)C(=O)N1CCN(c2nc(OC[C@@H]3CCCN3C)nc3c2CO[C@@H](c2cccc4cccc(C)c24)C3)C[C@@H]1CC#N ; C=C(F)C(=O)N1CCN(c2nc(OC[C@@H]3CCCN3C)nc3c2CO[C@H](c2cccc4cccc(C)c24)C3)C[C@@H]1CC#N ; C=C(F)C(=O)N1CCN(c2nc(OC[C@@H]3CCCN3C)nc3c2CO[C@@H](c2ccccc2C(F)(F)F)C3)C[C@@H]1CC#N ; C=C(F)C(=O)N1CCN(c2nc(OC[C@@H]3CCCN3C)nc3c2CO[C@H](c2ccccc2C(F)(F)F)C3)C[C@@H]1CC#N]

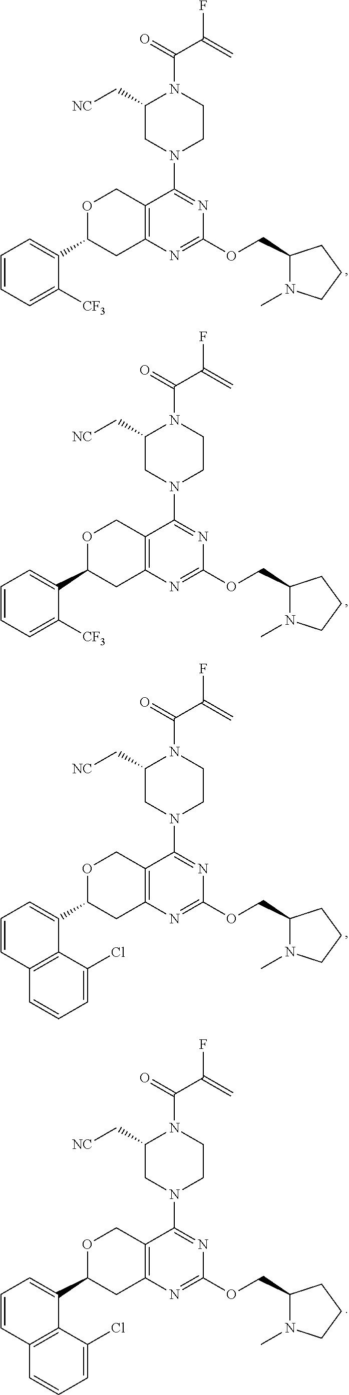 [SMILES: C=C(F)C(=O)N1CCN(c2nc(OC[C@H]3CCCN3C)nc3c2CO[C@@H](c2ccccc2C(F)(F)F)C3)C[C@@H]1CC#N ; C=C(F)C(=O)N1CCN(c2nc(OC[C@H]3CCCN3C)nc3c2CO[C@H](c2ccccc2C(F)(F)F)C3)C[C@@H]1CC#N ; C=C(F)C(=O)N1CCN(c2nc(OC[C@H]3CCCN3C)nc3c2CO[C@@H](c2cccc4cccc(Cl)c24)C3)C[C@@H]1CC#N ; C=C(F)C(=O)N1CCN(c2nc(OC[C@H]3CCCN3C)nc3c2CO[C@H](c2cccc4cccc(Cl)c24)C3)C[C@@H]1CC#N]

The present disclosure also provides a method for preparing the above-mentioned oxygen-containing heterocyclic compound represented by formula I, the method comprising route one or route two:

route one:

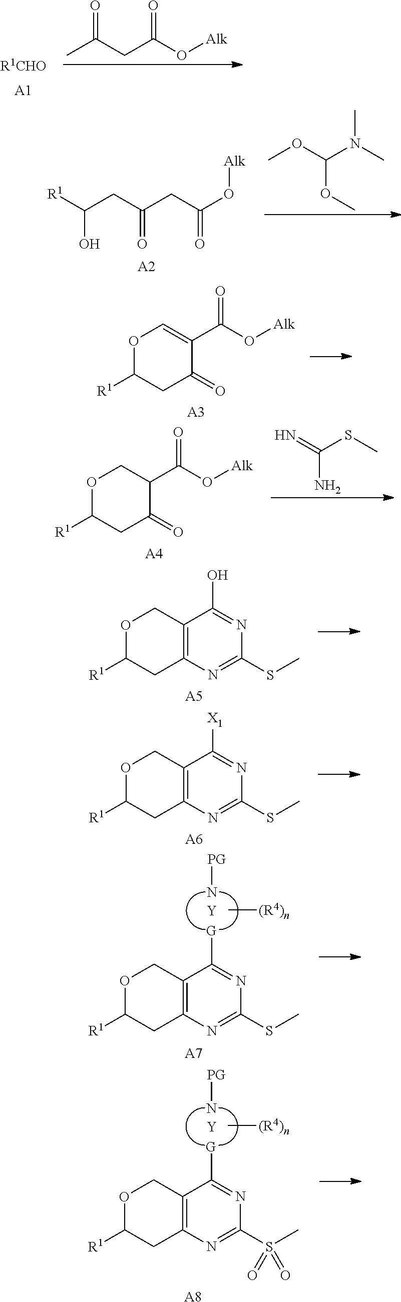

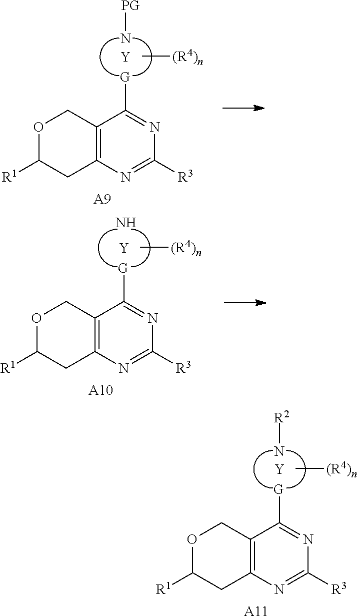

R¹, R², R³, R⁴, G, n and Y are all as defined above: X₁ is a leaving group (for example OTf or Cl): Alk is alkyl (for example, C₁₋₆ alkyl): PG is an amino protecting group (for example, Boc or Cbz);

route two:

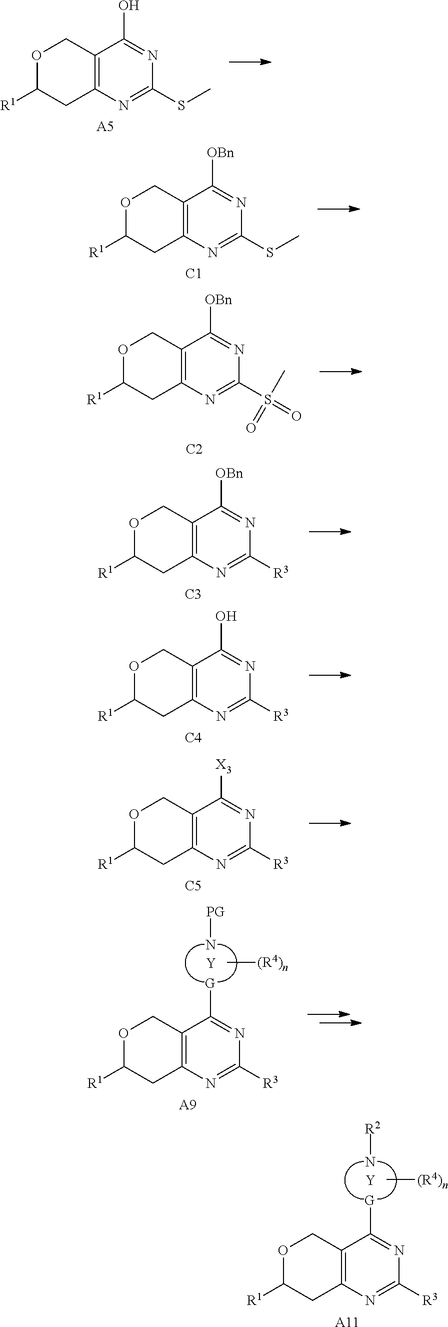

R¹, R², R³, R⁴, G, n and Y are all as defined above: X₃ is a leaving group (for example OTf or Cl): PG is an amino protecting group (for example, Boc or Cbz).

The route one is described in detail as follows: aldehyde compound A1 is condensed with acetyl acetate to obtain compound A2: A2 is condensed with DMF-DMA to obtain compound A3: A3 is reduced to A4: A5 is obtained from A4 after ring formation: the hydroxyl group in A5 is converted into a leaving group and A6 is obtained: A6 is converted into A7 by means of nucleophilic substitution, coupling, etc.: A7 is oxidized to obtain A8; A8 is further converted into A9: and A9 is deprotected and further converted into A11.

The route two is described in detail as follows: compound A5 is protected by Bn; C1 is oxidized to obtain C2: C2 is converted into C3 by nucleophilic substitution; C3 is subjected to Bn deprotection and converted into C4: the hydroxyl group in C4 is converted into a leaving group and C5 is obtained: C5 is converted into A9 by means of nucleophilic substitution, coupling, etc.; A9 is deprotected and further converted into A11.

The conditions and steps adopted for the chemical reactions involved in the various reaction routes described in the present disclosure all can be carried out with reference to conventional conditions and steps for such reactions in the art, and specific reference may be made to the literatures: R. Larock, Comprehensive Organic Transformations, VCH Publishers (1989): T. W. Greene and P. G. M. Nuts, Protective Groups in Organic Synthesis, 3^{rd} ED., John Wiley and Sons (1999): L. Fieser and M. Fieser, Fieser and Fieser's Reagents for Organic Synthesis. John Wiley and Sons (1994): L. Paquette, ed., Encyclopedia of Reagents for Organic Synthesis, John Wiley and Sons (1995) subsequent versions.

The present application cites the entire contents of the above literatures. In addition, other target compounds of the present disclosure can also be obtained from the compounds obtained by the above-mentioned methods through modifying peripheral positions with reference to related methods of the above literatures.

The present disclosure also provides a compound represented by formula A5, A6. A7, A8, A9, A10, C1, C2, C3, C4 or C5;

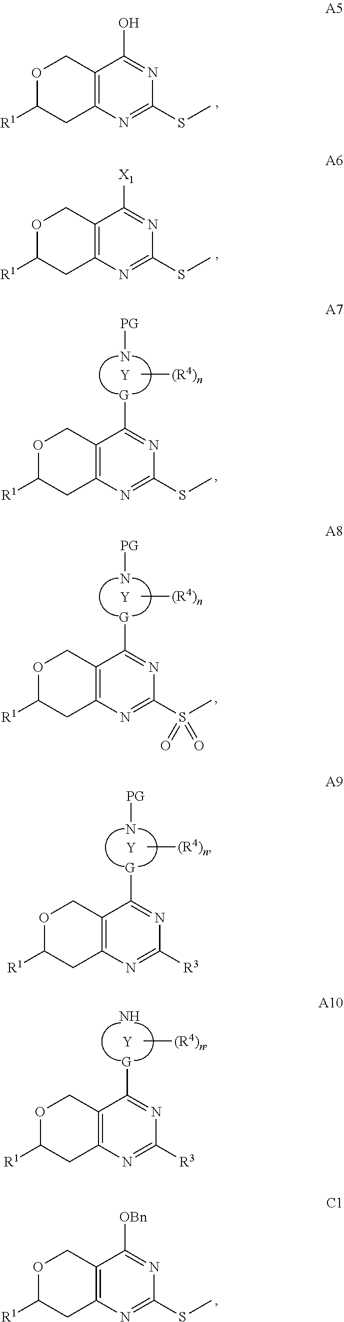

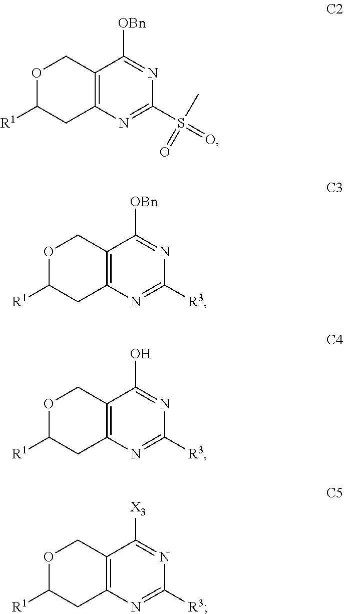

wherein R¹, R³, R⁴, G, Y and n are as defined above;

X¹ and X³ are independently leaving groups (for example OTf or Cl) PG is an amino protecting group (for example, Boc or Cbz).

In a certain embodiment, the compound represented by formula A5, A6, A7, A8, A9, A10, C1, C2, C3, C4 or C5 may be any one of the following compounds:

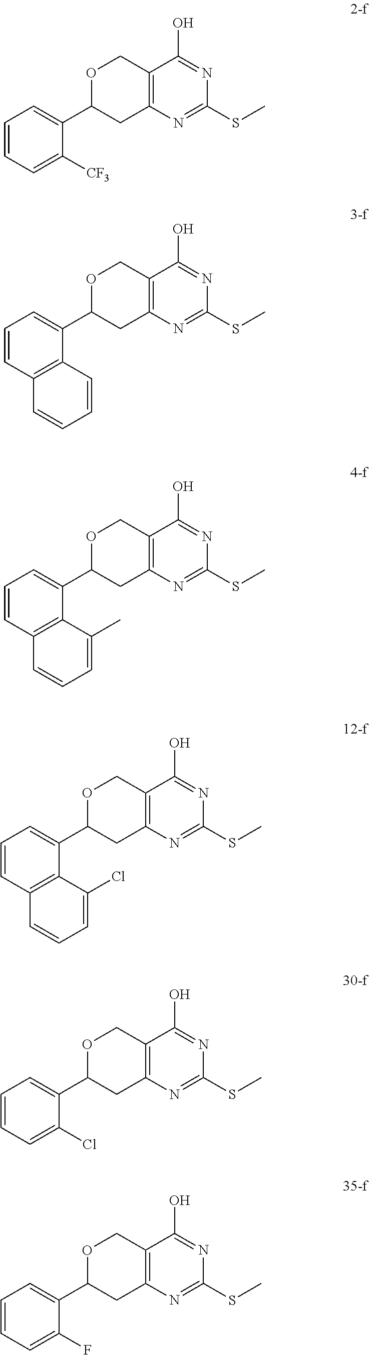 [SMILES: CSc1nc(O)c2c(n1)CC(c1ccccc1C(F)(F)F)OC2 ; CSc1nc(O)c2c(n1)CC(c1cccc3ccccc13)OC2 ; CSc1nc(O)c2c(n1)CC(c1cccc3cccc(C)c13)OC2 ; CSc1nc(O)c2c(n1)CC(c1cccc3cccc(Cl)c13)OC2 ; CSc1nc(O)c2c(n1)CC(c1ccccc1Cl)OC2 ; CSc1nc(O)c2c(n1)CC(c1ccccc1F)OC2]

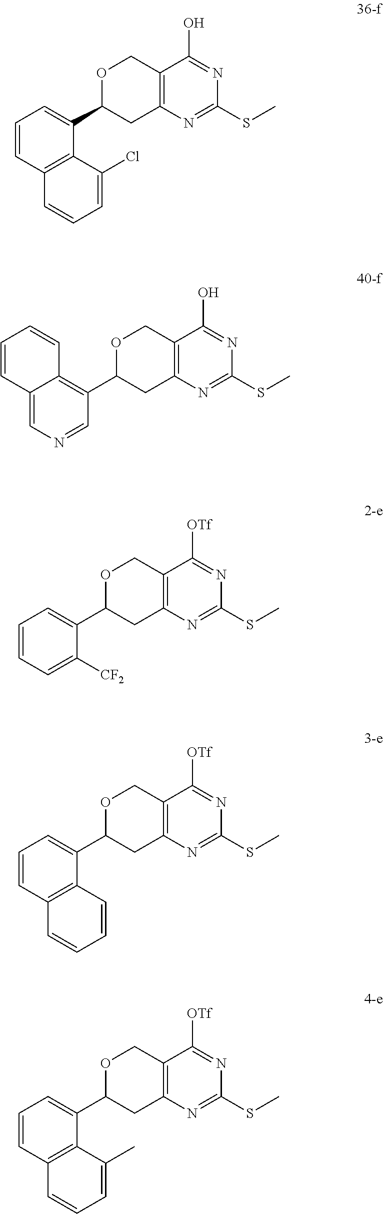

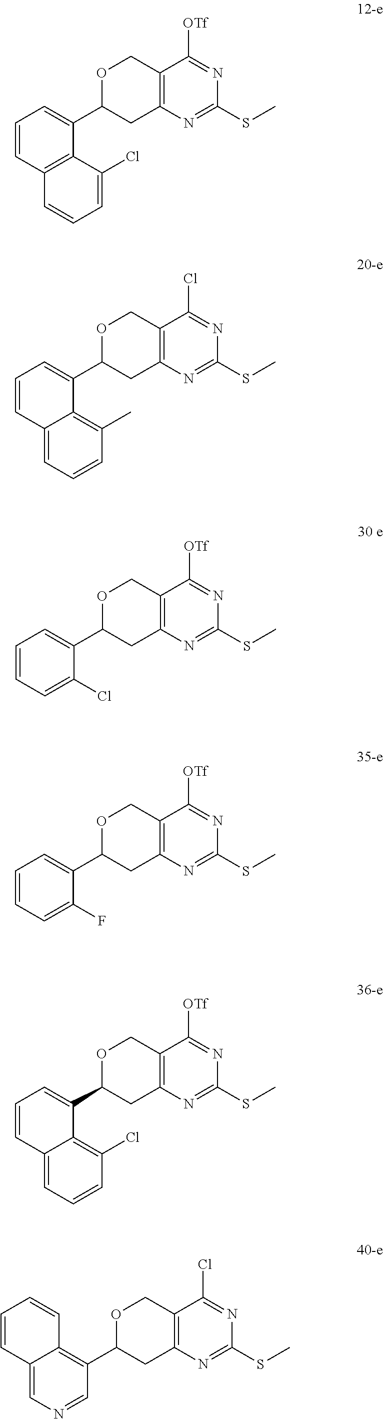 [SMILES: CSc1nc2c(c(OS(=O)(=O)C(F)(F)F)n1)COC(c1cccc3cccc(Cl)c13)C2 ; CSc1nc(Cl)c2c(n1)CC(c1cccc3cccc(C)c13)OC2 ; CSc1nc2c(c(OS(=O)(=O)C(F)(F)F)n1)COC(c1ccccc1Cl)C2 ; CSc1nc2c(c(OS(=O)(=O)C(F)(F)F)n1)COC(c1ccccc1F)C2 ; CSc1nc2c(c(OS(=O)(=O)C(F)(F)F)n1)CO[C@H](c1cccc3cccc(Cl)c13)C2 ; CSc1nc(Cl)c2c(n1)CC(c1cncc3ccccc13)OC2]

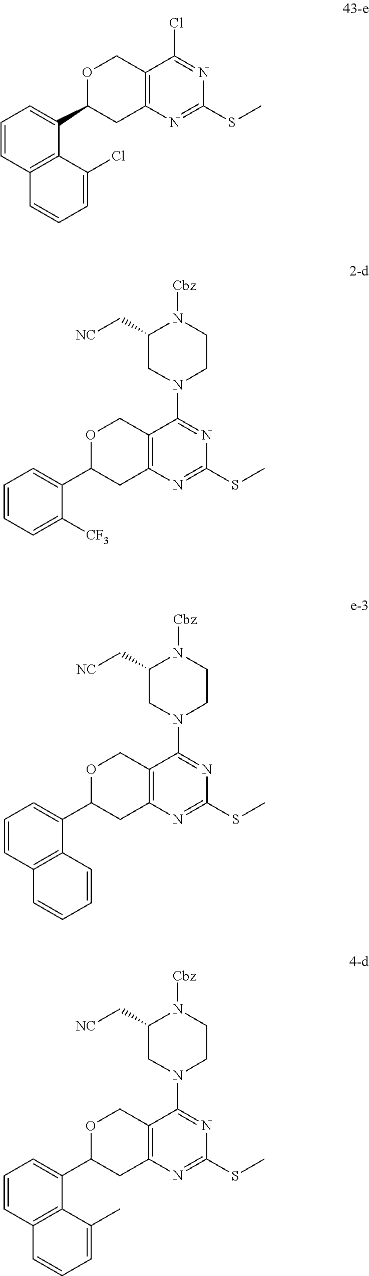 [SMILES: CSc1nc(Cl)c2c(n1)C[C@@H](c1cccc3cccc(Cl)c13)OC2 ; CSc1nc2c(c(N3CCN(C(=O)OCc4ccccc4)[C@@H](CC#N)C3)n1)COC(c1ccccc1C(F)(F)F)C2 ; CSc1nc2c(c(N3CCN(C(=O)OCc4ccccc4)[C@@H](CC#N)C3)n1)COC(c1cccc3ccccc13)C2 ; CSc1nc2c(c(N3CCN(C(=O)OCc4ccccc4)[C@@H](CC#N)C3)n1)COC(c1cccc3cccc(C)c13)C2]

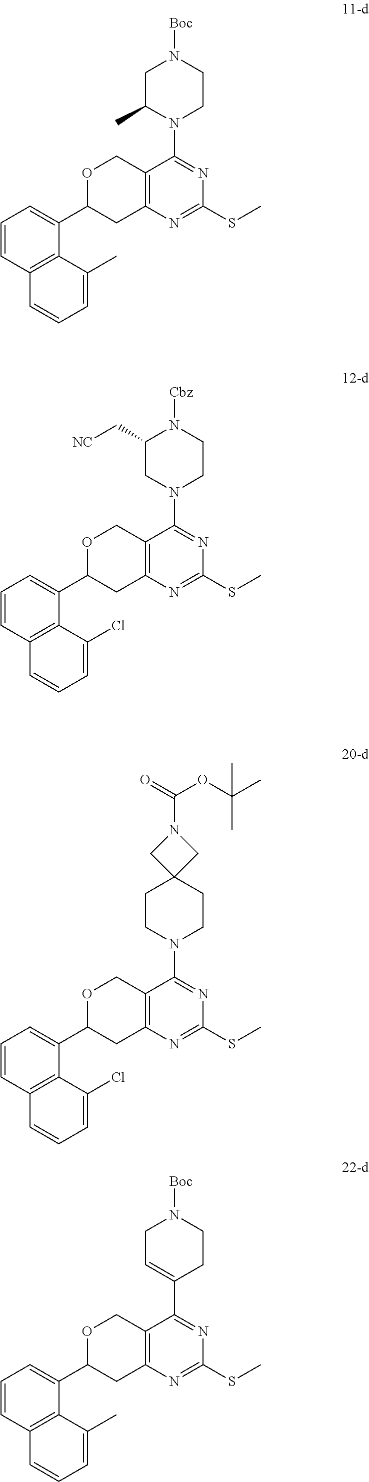 [SMILES: CSc1nc2c(c(N3CCN(C(=O)OC(C)(C)C)C[C@@H]3C)n1)COC(c1cccc3cccc(C)c13)C2 ; CSc1nc2c(c(N3CCN(C(=O)OCc4ccccc4)[C@@H](CC#N)C3)n1)COC(c1cccc3cccc(Cl)c13)C2 ; CSc1nc2c(c(N3CCC4(CC3)CN(C(=O)OC(C)(C)C)C4)n1)COC(c1cccc3cccc(Cl)c13)C2 ; CSc1nc2c(c(C3=CCN(C(=O)OC(C)(C)C)CC3)n1)COC(c1cccc3cccc(C)c13)C2]

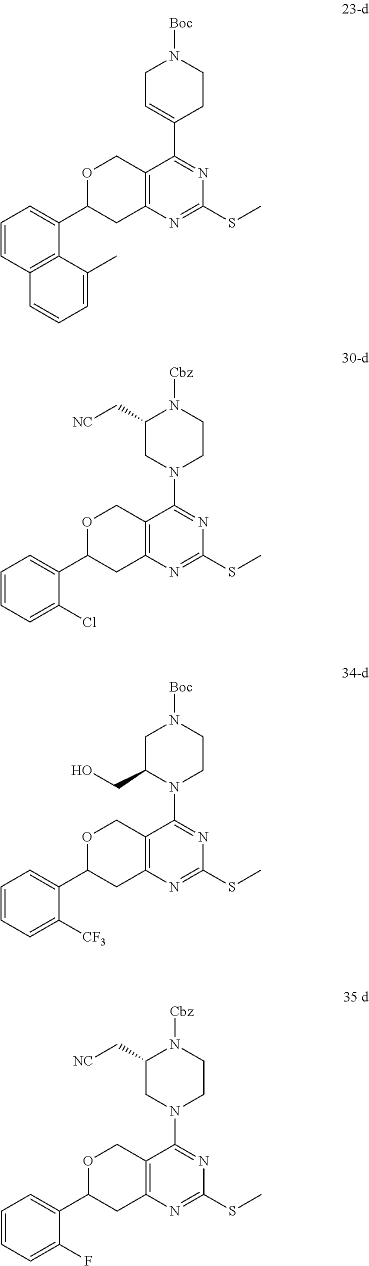 [SMILES: CSc1nc2c(c(C3=CCN(C(=O)OC(C)(C)C)CC3)n1)COC(c1cccc3cccc(C)c13)C2 ; CSc1nc2c(c(N3CCN(C(=O)OCc4ccccc4)[C@@H](CC#N)C3)n1)COC(c1ccccc1Cl)C2 ; CSc1nc2c(c(N3CCN(C(=O)OC(C)(C)C)C[C@@H]3CO)n1)COC(c1ccccc1C(F)(F)F)C2 ; CSc1nc2c(c(N3CCN(C(=O)OCc4ccccc4)[C@@H](CC#N)C3)n1)COC(c1ccccc1F)C2]

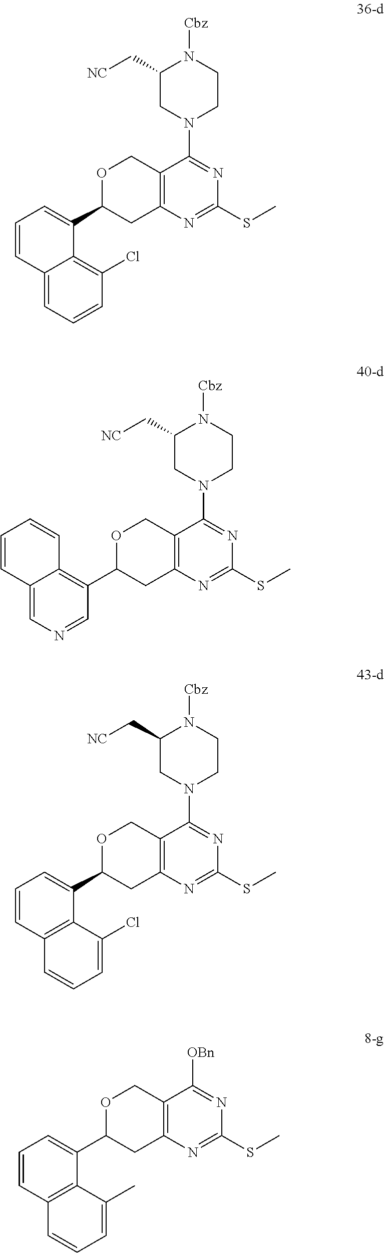 [SMILES: CSc1nc2c(c(N3CCN(C(=O)OCc4ccccc4)[C@@H](CC#N)C3)n1)CO[C@H](c1cccc3cccc(Cl)c13)C2 ; CSc1nc2c(c(N3CCN(C(=O)OCc4ccccc4)[C@@H](CC#N)C3)n1)COC(c1cncc3ccccc13)C2 ; CSc1nc2c(c(N3CCN(C(=O)OCc4ccccc4)[C@H](CC#N)C3)n1)CO[C@H](c1cccc3cccc(Cl)c13)C2 ; CSc1nc2c(c(OCc3ccccc3)n1)COC(c1cccc3cccc(C)c13)C2]

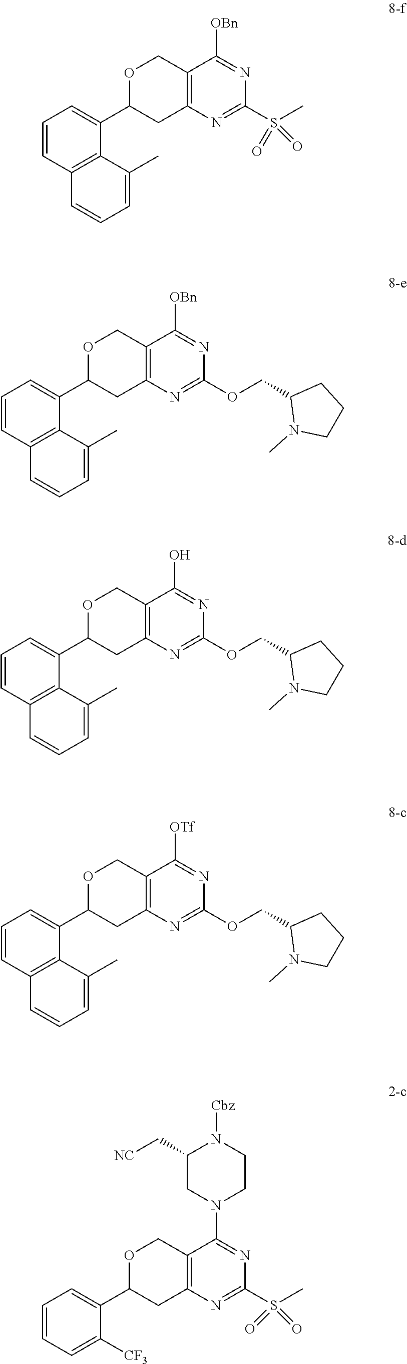

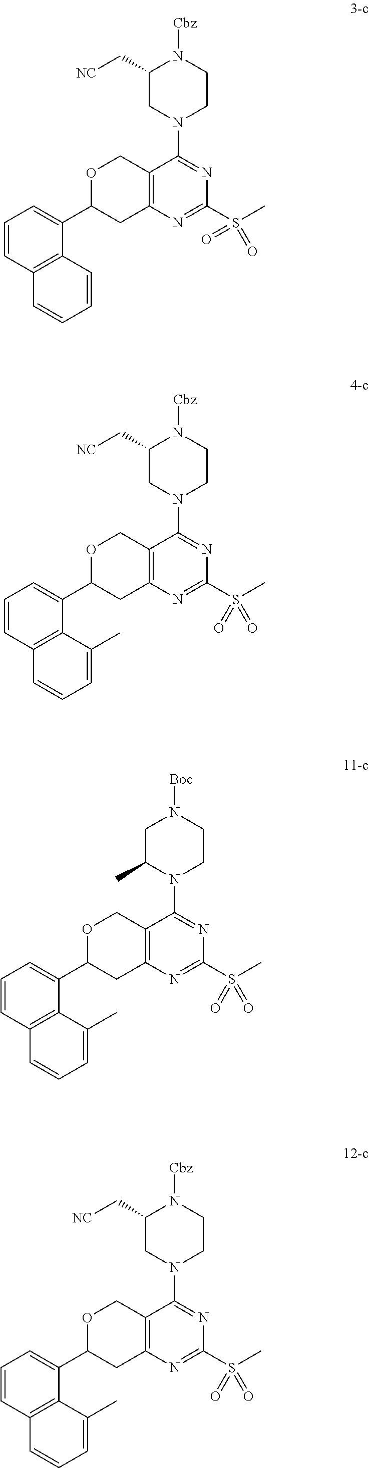 [SMILES: CS(=O)(=O)c1nc2c(c(N3CCN(C(=O)OCc4ccccc4)[C@@H](CC#N)C3)n1)COC(c1cccc3ccccc13)C2 ; Cc1cccc2cccc(C3Cc4nc(S(C)(=O)=O)nc(N5CCN(C(=O)OCc6ccccc6)[C@@H](CC#N)C5)c4CO3)c12 ; Cc1cccc2cccc(C3Cc4nc(S(C)(=O)=O)nc(N5CCN(C(=O)OC(C)(C)C)C[C@@H]5C)c4CO3)c12 ; Cc1cccc2cccc(C3Cc4nc(S(C)(=O)=O)nc(N5CCN(C(=O)OCc6ccccc6)[C@@H](CC#N)C5)c4CO3)c12]

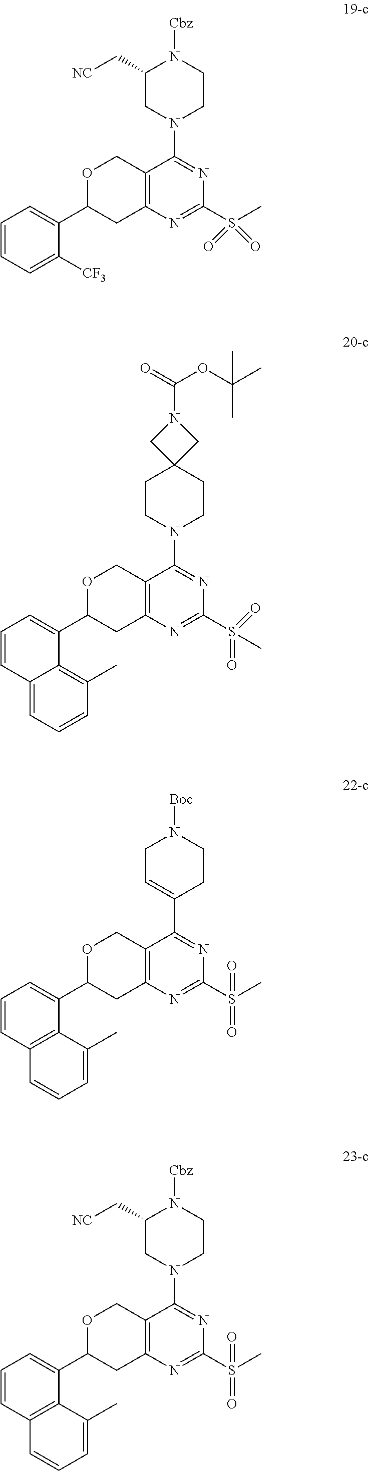 [SMILES: CS(=O)(=O)c1nc2c(c(N3CCN(C(=O)OCc4ccccc4)[C@@H](CC#N)C3)n1)COC(c1ccccc1C(F)(F)F)C2 ; Cc1cccc2cccc(C3Cc4nc(S(C)(=O)=O)nc(N5CCC6(CC5)CN(C(=O)OC(C)(C)C)C6)c4CO3)c12 ; Cc1cccc2cccc(C3Cc4nc(S(C)(=O)=O)nc(C5=CCN(C(=O)OC(C)(C)C)CC5)c4CO3)c12 ; Cc1cccc2cccc(C3Cc4nc(S(C)(=O)=O)nc(N5CCN(C(=O)OCc6ccccc6)[C@@H](CC#N)C5)c4CO3)c12]

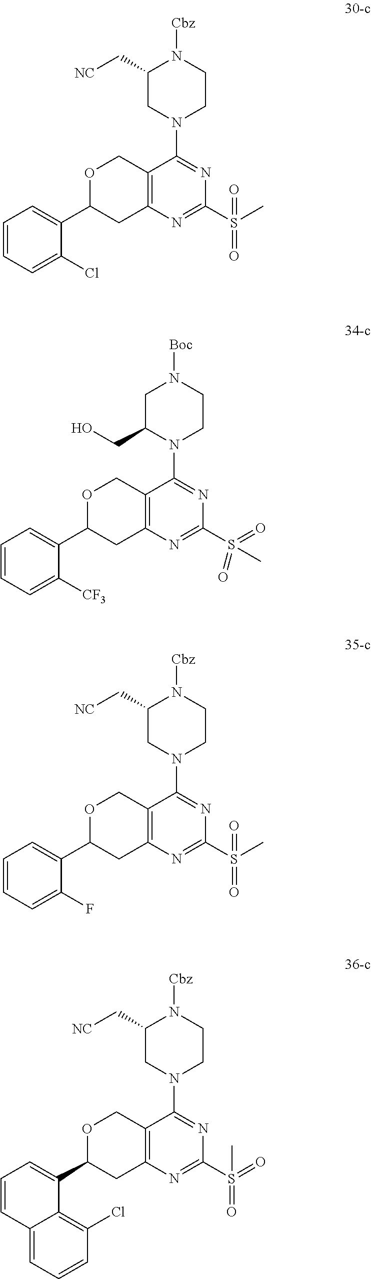 [SMILES: CS(=O)(=O)c1nc2c(c(N3CCN(C(=O)OCc4ccccc4)[C@@H](CC#N)C3)n1)COC(c1ccccc1Cl)C2 ; CC(C)(C)OC(=O)N1CCN(c2nc(S(C)(=O)=O)nc3c2COC(c2ccccc2C(F)(F)F)C3)[C@@H](CO)C1 ; CS(=O)(=O)c1nc2c(c(N3CCN(C(=O)OCc4ccccc4)[C@@H](CC#N)C3)n1)COC(c1ccccc1F)C2 ; CS(=O)(=O)c1nc2c(c(N3CCN(C(=O)OCc4ccccc4)[C@@H](CC#N)C3)n1)CO[C@H](c1cccc3cccc(Cl)c13)C2]

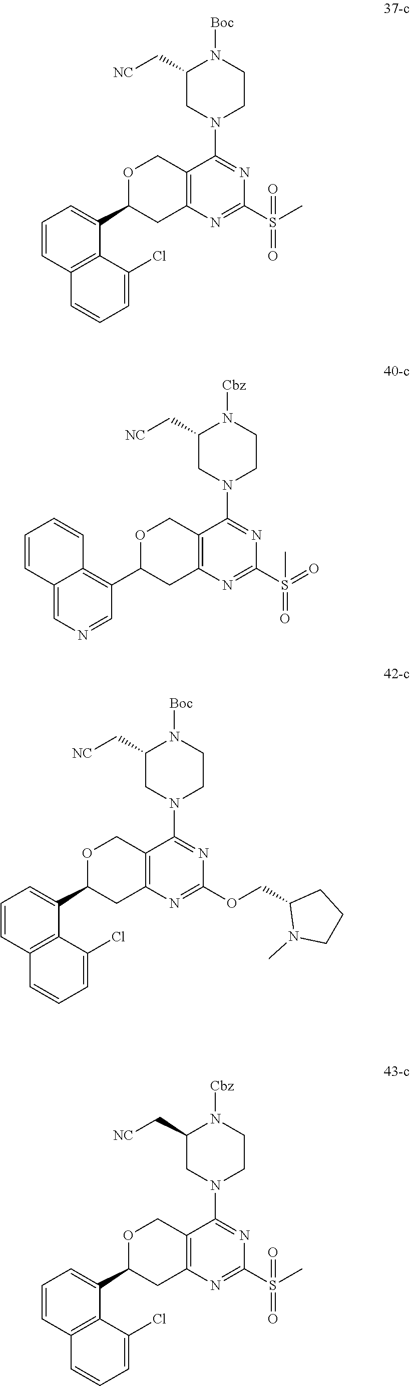 [SMILES: CC(C)(C)OC(=O)N1CCN(c2nc(S(C)(=O)=O)nc3c2CO[C@H](c2cccc4cccc(Cl)c24)C3)C[C@@H]1CC#N ; CS(=O)(=O)c1nc2c(c(N3CCN(C(=O)OCc4ccccc4)[C@@H](CC#N)C3)n1)COC(c1cncc3ccccc13)C2 ; CN1CCC[C@H]1COc1nc2c(c(N3CCN(C(=O)OC(C)(C)C)[C@@H](CC#N)C3)n1)CO[C@H](c1cccc3cccc(Cl)c13)C2 ; CS(=O)(=O)c1nc2c(c(N3CCN(C(=O)OCc4ccccc4)[C@H](CC#N)C3)n1)CO[C@H](c1cccc3cccc(Cl)c13)C2]

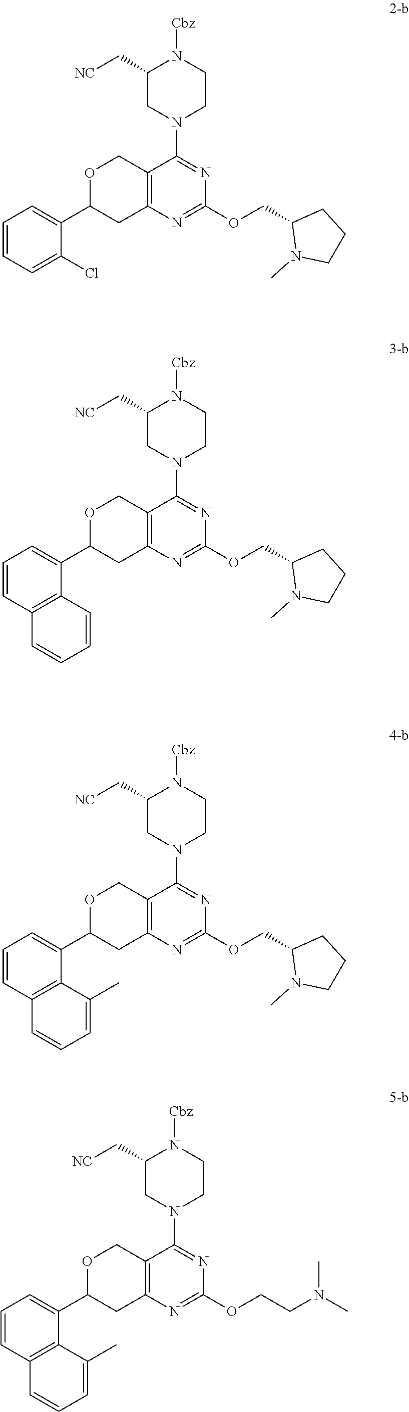 [SMILES: CN1CCC[C@H]1COc1nc2c(c(N3CCN(C(=O)OCc4ccccc4)[C@@H](CC#N)C3)n1)COC(c1ccccc1Cl)C2 ; CN1CCC[C@H]1COc1nc2c(c(N3CCN(C(=O)OCc4ccccc4)[C@@H](CC#N)C3)n1)COC(c1cccc3ccccc13)C2 ; Cc1cccc2cccc(C3Cc4nc(OC[C@@H]5CCCN5C)nc(N5CCN(C(=O)OCc6ccccc6)[C@@H](CC#N)C5)c4CO3)c12 ; Cc1cccc2cccc(C3Cc4nc(OCCN(C)C)nc(N5CCN(C(=O)OCc6ccccc6)[C@@H](CC#N)C5)c4CO3)c12]

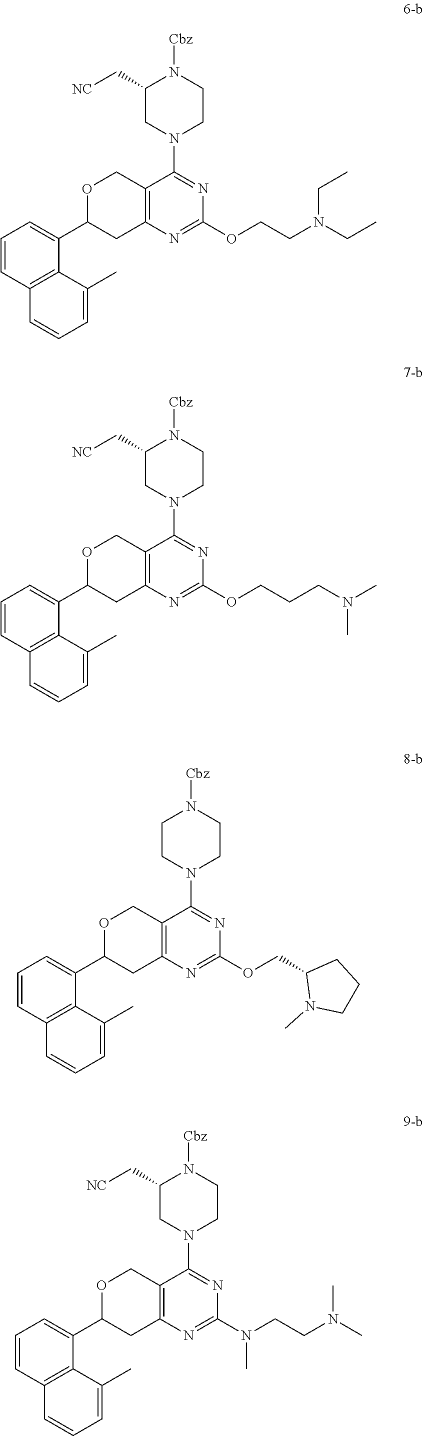 [SMILES: CCN(CC)CCOc1nc2c(c(N3CCN(C(=O)OCc4ccccc4)[C@@H](CC#N)C3)n1)COC(c1cccc3cccc(C)c13)C2 ; Cc1cccc2cccc(C3Cc4nc(OCCCN(C)C)nc(N5CCN(C(=O)OCc6ccccc6)[C@@H](CC#N)C5)c4CO3)c12 ; Cc1cccc2cccc(C3Cc4nc(OC[C@@H]5CCCN5C)nc(N5CCN(C(=O)OCc6ccccc6)CC5)c4CO3)c12 ; Cc1cccc2cccc(C3Cc4nc(N(C)CCN(C)C)nc(N5CCN(C(=O)OCc6ccccc6)[C@@H](CC#N)C5)c4CO3)c12]

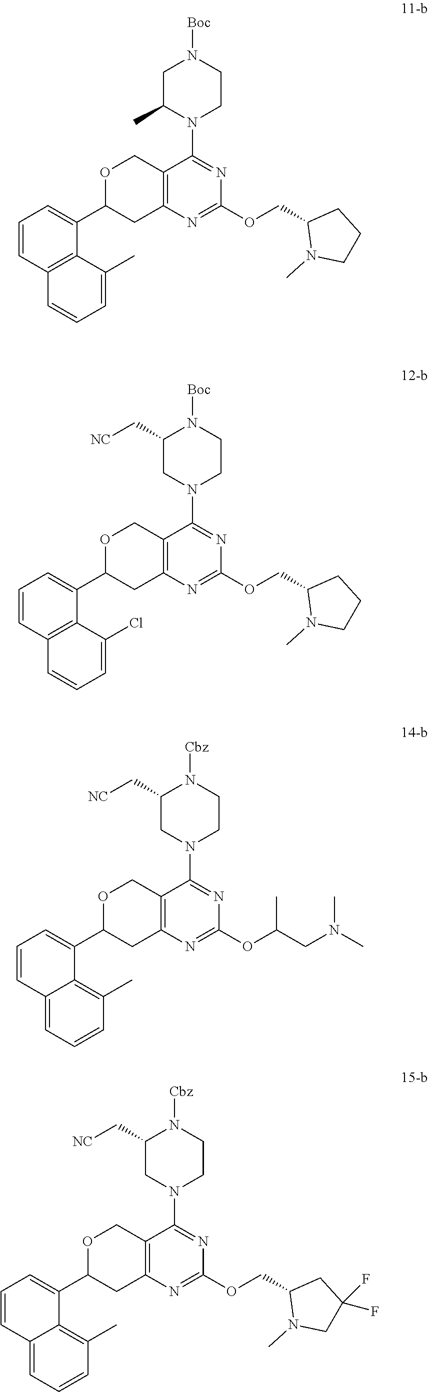 [SMILES: Cc1cccc2cccc(C3Cc4nc(OC[C@@H]5CCCN5C)nc(N5CCN(C(=O)OC(C)(C)C)C[C@@H]5C)c4CO3)c12 ; CN1CCC[C@H]1COc1nc2c(c(N3CCN(C(=O)OC(C)(C)C)[C@@H](CC#N)C3)n1)COC(c1cccc3cccc(Cl)c13)C2 ; Cc1cccc2cccc(C3Cc4nc(OC(C)CN(C)C)nc(N5CCN(C(=O)OCc6ccccc6)[C@@H](CC#N)C5)c4CO3)c12 ; Cc1cccc2cccc(C3Cc4nc(OC[C@@H]5CC(F)(F)CN5C)nc(N5CCN(C(=O)OCc6ccccc6)[C@@H](CC#N)C5)c4CO3)c12]

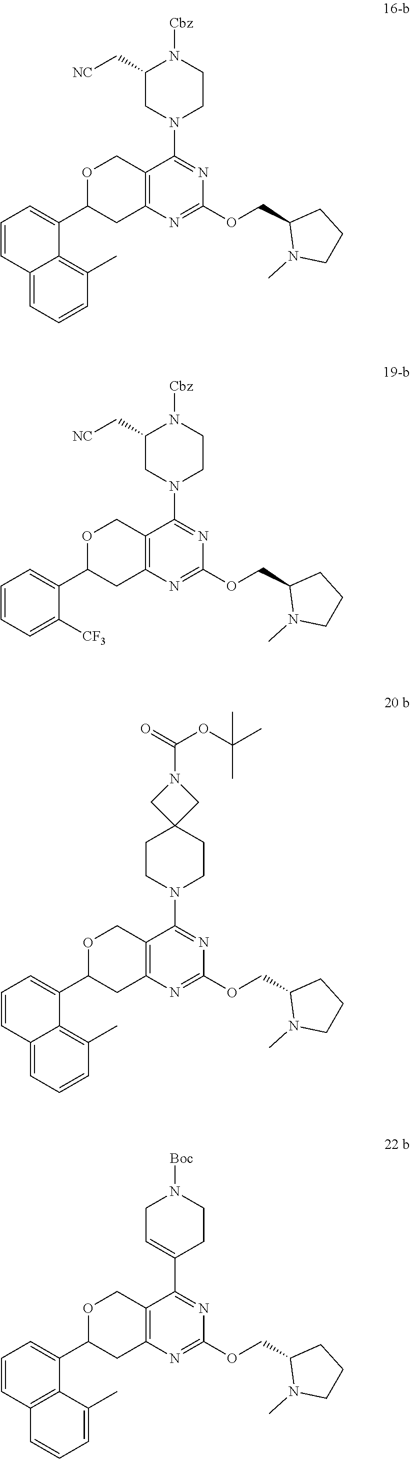 [SMILES: Cc1cccc2cccc(C3Cc4nc(OC[C@H]5CCCN5C)nc(N5CCN(C(=O)OCc6ccccc6)[C@@H](CC#N)C5)c4CO3)c12 ; CN1CCC[C@@H]1COc1nc2c(c(N3CCN(C(=O)OCc4ccccc4)[C@@H](CC#N)C3)n1)COC(c1ccccc1C(F)(F)F)C2 ; Cc1cccc2cccc(C3Cc4nc(OC[C@@H]5CCCN5C)nc(N5CCC6(CC5)CN(C(=O)OC(C)(C)C)C6)c4CO3)c12 ; Cc1cccc2cccc(C3Cc4nc(OC[C@@H]5CCCN5C)nc(C5=CCN(C(=O)OC(C)(C)C)CC5)c4CO3)c12]

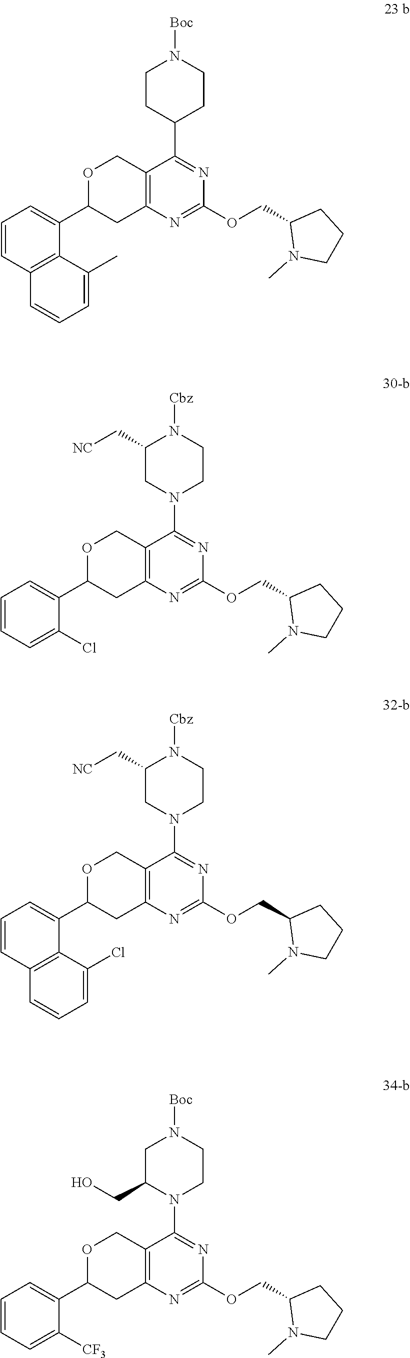 [SMILES: Cc1cccc2cccc(C3Cc4nc(OC[C@@H]5CCCN5C)nc(C5CCN(C(=O)OC(C)(C)C)CC5)c4CO3)c12 ; CN1CCC[C@H]1COc1nc2c(c(N3CCN(C(=O)OCc4ccccc4)[C@@H](CC#N)C3)n1)COC(c1ccccc1Cl)C2 ; CN1CCC[C@@H]1COc1nc2c(c(N3CCN(C(=O)OCc4ccccc4)[C@@H](CC#N)C3)n1)COC(c1cccc3cccc(Cl)c13)C2 ; CN1CCC[C@H]1COc1nc2c(c(N3CCN(C(=O)OC(C)(C)C)C[C@@H]3CO)n1)COC(c1ccccc1C(F)(F)F)C2]

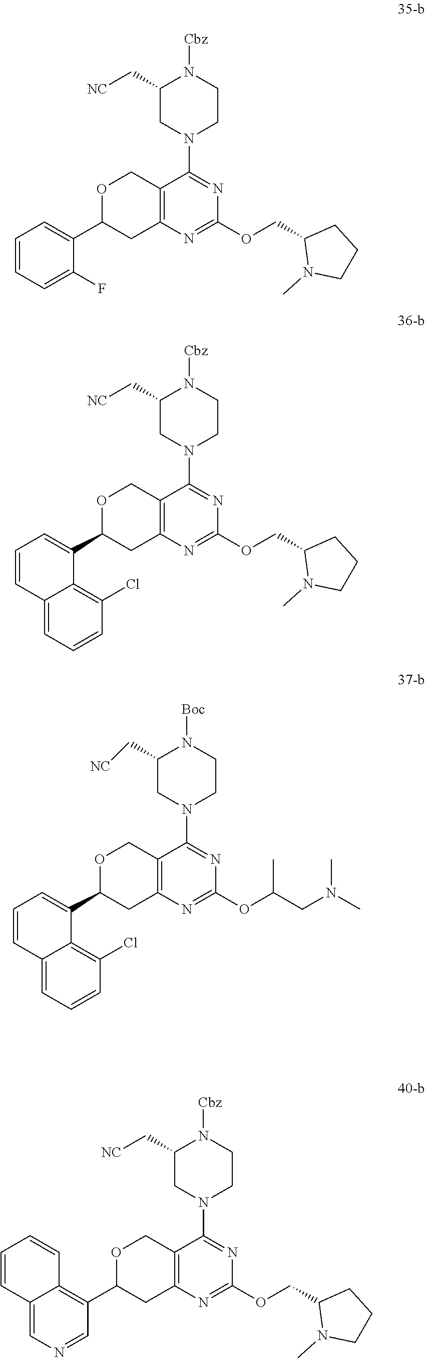

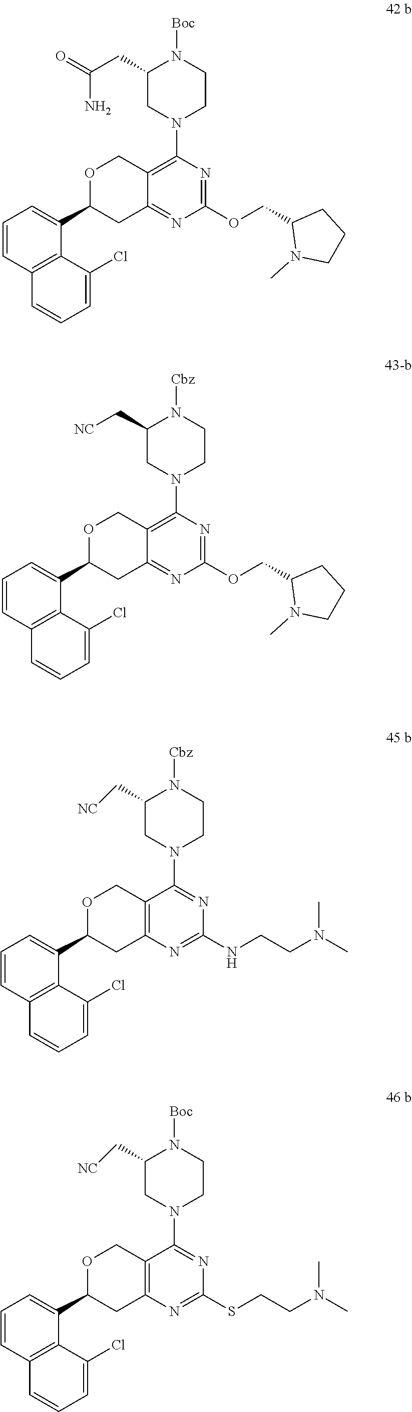

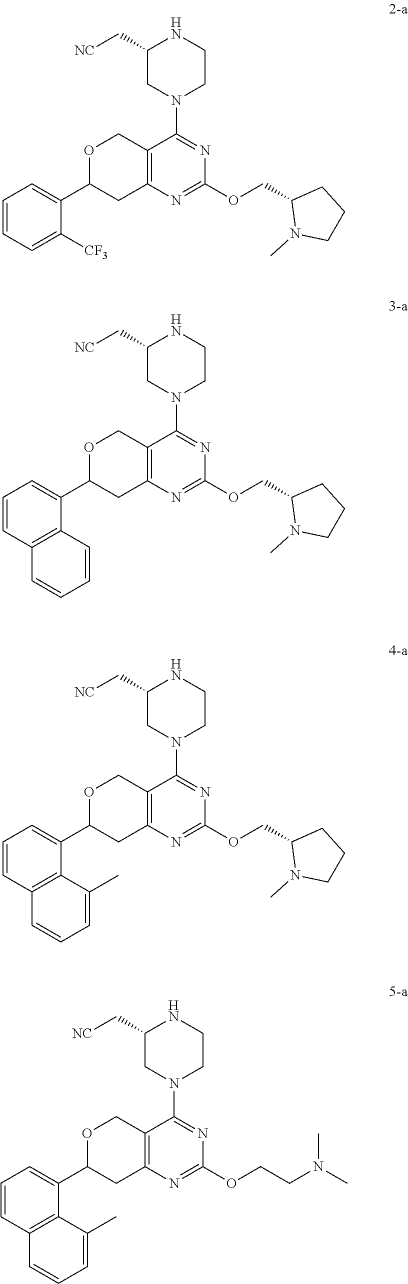 [SMILES: CN1CCC[C@H]1COc1nc2c(c(N3CCN[C@@H](CC#N)C3)n1)COC(c1ccccc1C(F)(F)F)C2 ; CN1CCC[C@H]1COc1nc2c(c(N3CCN[C@@H](CC#N)C3)n1)COC(c1cccc3ccccc13)C2 ; Cc1cccc2cccc(C3Cc4nc(OC[C@@H]5CCCN5C)nc(N5CCN[C@@H](CC#N)C5)c4CO3)c12 ; Cc1cccc2cccc(C3Cc4nc(OCCN(C)C)nc(N5CCN[C@@H](CC#N)C5)c4CO3)c12]

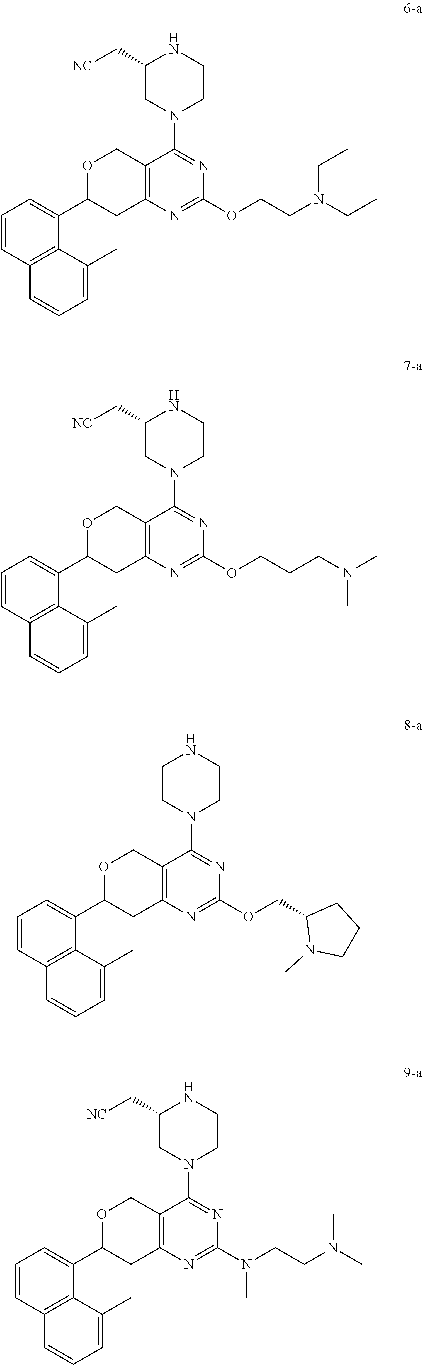 [SMILES: CCN(CC)CCOc1nc2c(c(N3CCN[C@@H](CC#N)C3)n1)COC(c1cccc3cccc(C)c13)C2 ; Cc1cccc2cccc(C3Cc4nc(OCCCN(C)C)nc(N5CCN[C@@H](CC#N)C5)c4CO3)c12 ; Cc1cccc2cccc(C3Cc4nc(OC[C@@H]5CCCN5C)nc(N5CCNCC5)c4CO3)c12 ; Cc1cccc2cccc(C3Cc4nc(N(C)CCN(C)C)nc(N5CCN[C@@H](CC#N)C5)c4CO3)c12]

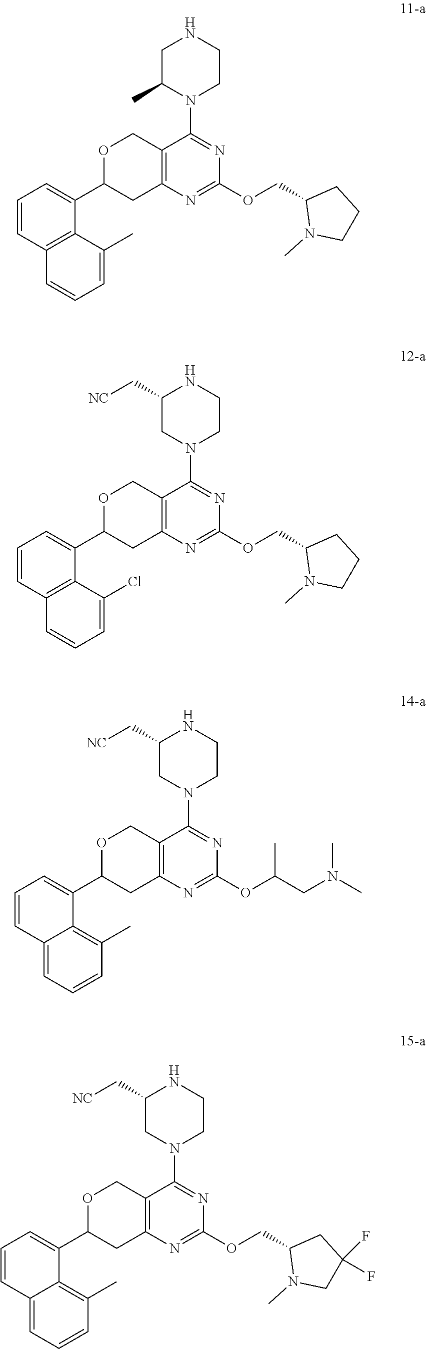 [SMILES: Cc1cccc2cccc(C3Cc4nc(OC[C@@H]5CCCN5C)nc(N5CCNC[C@@H]5C)c4CO3)c12 ; CN1CCC[C@H]1COc1nc2c(c(N3CCN[C@@H](CC#N)C3)n1)COC(c1cccc3cccc(Cl)c13)C2 ; Cc1cccc2cccc(C3Cc4nc(OC(C)CN(C)C)nc(N5CCN[C@@H](CC#N)C5)c4CO3)c12 ; Cc1cccc2cccc(C3Cc4nc(OC[C@@H]5CC(F)(F)CN5C)nc(N5CCN[C@@H](CC#N)C5)c4CO3)c12]

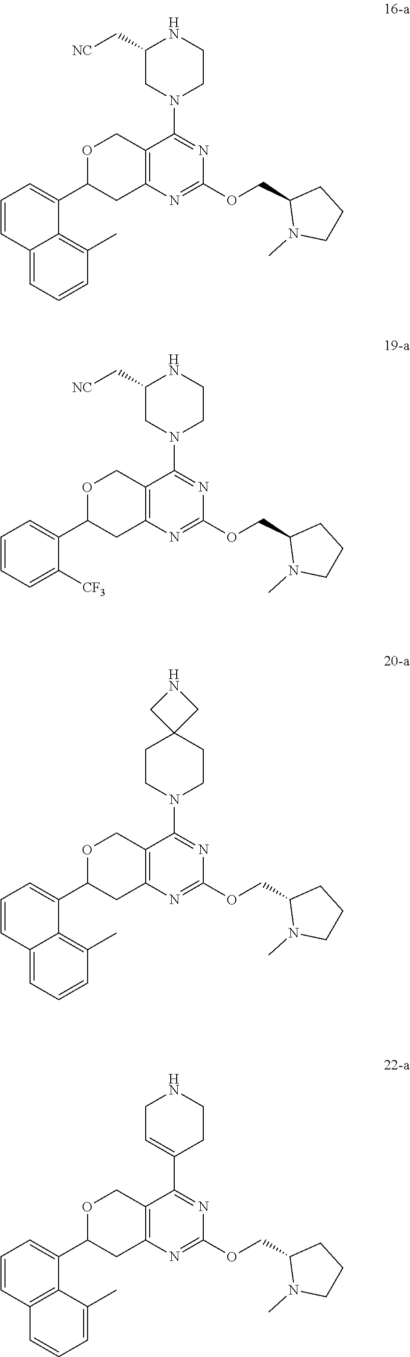 [SMILES: Cc1cccc2cccc(C3Cc4nc(OC[C@H]5CCCN5C)nc(N5CCN[C@@H](CC#N)C5)c4CO3)c12 ; CN1CCC[C@@H]1COc1nc2c(c(N3CCN[C@@H](CC#N)C3)n1)COC(c1ccccc1C(F)(F)F)C2 ; Cc1cccc2cccc(C3Cc4nc(OC[C@@H]5CCCN5C)nc(N5CCC6(CC5)CNC6)c4CO3)c12 ; Cc1cccc2cccc(C3Cc4nc(OC[C@@H]5CCCN5C)nc(C5=CCNCC5)c4CO3)c12]

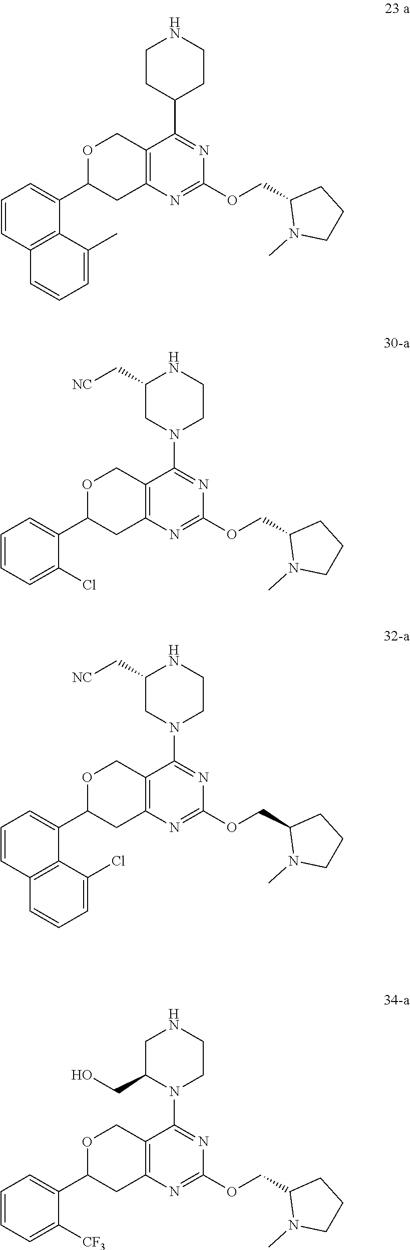

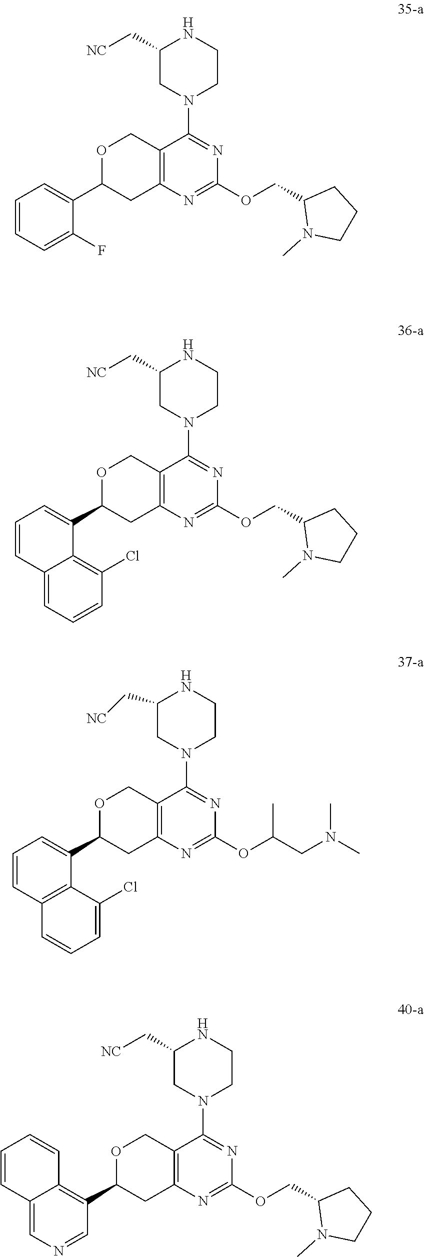

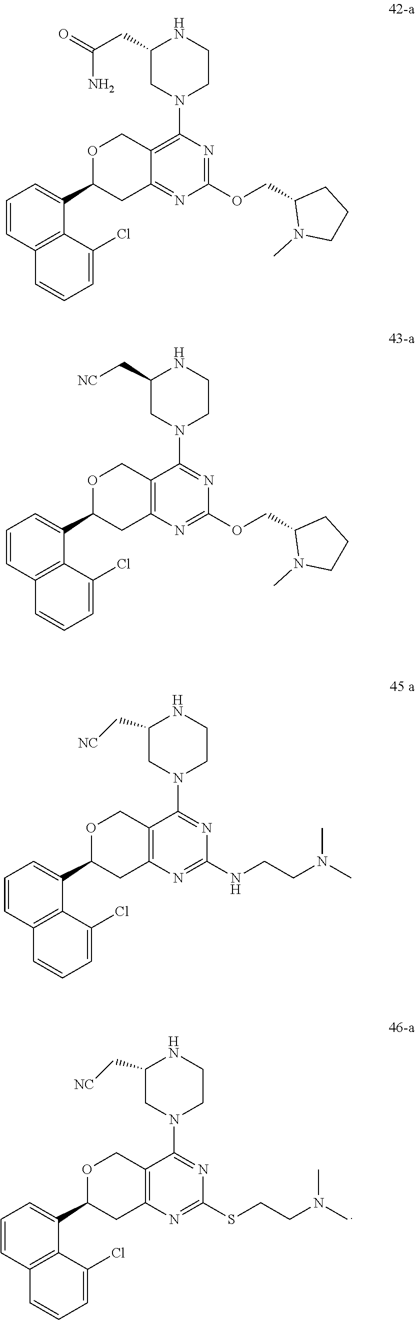 [SMILES: CN1CCC[C@H]1COc1nc2c(c(N3CCN[C@@H](CC(N)=O)C3)n1)CO[C@H](c1cccc3cccc(Cl)c13)C2 ; CN1CCC[C@H]1COc1nc2c(c(N3CCN[C@H](CC#N)C3)n1)CO[C@H](c1cccc3cccc(Cl)c13)C2 ; CN(C)CCNc1nc2c(c(N3CCN[C@@H](CC#N)C3)n1)CO[C@H](c1cccc3cccc(Cl)c13)C2 ; CN(C)CCSc1nc2c(c(N3CCN[C@@H](CC#N)C3)n1)CO[C@H](c1cccc3cccc(Cl)c13)C2]

The present disclosure also provides a pharmaceutical composition, comprising substance A and a pharmaceutical adjuvant: the substance A is the above-mentioned oxygen-containing heterocyclic compound represented by formula I, a pharmaceutically acceptable salt thereof, a stereoisomer thereof, a tautomer thereof or an isotopic compound thereof.

The present disclosure also provides use of substance A in the preparation of an RAS inhibitor, wherein the substance A is the above-mentioned oxygen-containing heterocyclic compound represented by formula I, a pharmaceutically acceptable salt thereof, a stereoisomer thereof, a tautomer thereof or an isotopic compound thereof.

The RAS comprises, for example, KRAS G12C. HRAS G12C or NRAS G12C mutation; for example, KRAS G12C.

The present disclosure also provides use of substance A in the preparation of a medicament, wherein the medicament is used to treat or prevent an RAS-mediated disease:

the substance A is the above-mentioned oxygen-containing heterocyclic compound represented by formula I, a pharmaceutically acceptable salt thereof, a stereoisomer thereof, a tautomer thereof or an isotopic compound thereof.

The RAS comprises, for example, KRAS G12C. HRAS G12C or NRAS G12C mutation; for example, KRAS G12C.

The RAS-mediated disease is, for example, cancer. The cancer is, for example, one or more of colon cancer, pancreatic cancer, breast cancer, prostate cancer, lung cancer, brain cancer, ovarian cancer, cervical cancer, testicular cancer, renal carcinoma, head or neck cancer, bone cancer, skin cancer, rectal cancer, liver cancer, colon cancer, esophageal cancer, gastric cancer, pancreatic cancer, thyroid cancer, bladder cancer, lymphoma, leukemia and melanoma.

The present disclosure also provides use of substance A in the manufacture of a medicament, wherein the medicament is used to treat or prevent cancer;

the substance A is the above-mentioned oxygen-containing heterocyclic compound represented by formula I, a pharmaceutically acceptable salt thereof, a stereoisomer thereof, a tautomer thereof or an isotopic compound thereof.

The cancer is, for example, one or more of colon cancer, pancreatic cancel, breast cancer, prostate cancer, lung cancer, brain cancer, ovarian cancer, cervical cancer, testicular cancer, renal carcinoma, head or neck cancel, bone cancer, skin cancer, rectal cancer, liver cancer, colon cancer, esophageal cancer, gastric cancel, pancreatic cancer, thyroid cancer, bladder cancer, lymphoma, leukemia and melanoma.

The present disclosure also provides an oxygen-containing heterocyclic compound represented by formula I, a pharmaceutically acceptable salt thereof, a solvate thereof, a solvate of the pharmaceutically acceptable salt thereof, a crystal form thereof a stereoisomer thereof, a tautomer thereof or an isotopic compound thereof;

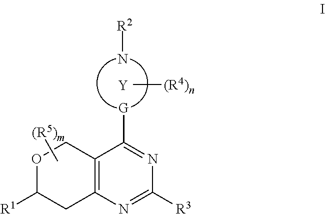

wherein R¹ is C₆₋₂₀ aryl. “5-12 membered heteroaryl containing 1-4 heteroatoms selected from one or more of O, S and N”. C₆₋₂₀ aryl substituted with one or more R¹⁻⁶, or “5-12 membered heteroaryl containing 1-4 heteroatoms selected from one or more of O, S and N” substituted with one or more R¹⁻⁷;

R¹⁻⁶ and R¹⁻⁷ are independently halogen, hydroxyl, —C(═O)R⁶⁵, —NR⁶³R⁶⁴, —C(═O)OR⁶⁶, —C(═O)NR⁶⁹R⁶¹⁰, C₁₋₆ alkyl, C₁₋₆ alkoxy. C₃₋₁₀ cycloalkyl, “5-7 membered heterocycloalkyl containing 1 or 2 heteroatoms selected from one or more of O and N”. C₆₋₂₀ aryl. “5-7 membered heteroaryl containing 1 or 2 heteroatoms selected from one or more of 0 and N”. C₁₋₆ alkyl substituted with one or more R¹⁻⁶⁻¹, C₁₋₆ alkoxy substituted with one or more R¹⁻⁶⁻². C₃₋₁₀ cycloalkyl substituted with one or more R¹⁻⁶⁻³. “5-7 membered heterocycloalkyl containing 1 or 2 heteroatoms selected from one or more of O and N” substituted with one or more R¹⁻⁶⁻⁴. C₆₋₂₀ aryl substituted with one or more R¹⁻⁶⁻⁵, or “5-7 membered heteroaryl containing 1 or 2 heteroatoms selected from one or more of O and N” substituted with one or more R¹⁻⁶⁻⁶;

R¹⁻⁶⁻¹, R¹⁻⁶⁻², R¹⁻⁶⁻³, R¹⁻⁶⁻⁴, R¹⁻⁶⁻⁵ and R¹⁻⁶⁻⁶ are independently cyano, halogen, hydroxyl, C₁₋₆ alkoxy. C₁₋₆ alkyl, —C(═O)R⁶⁵⁻², —NR⁶³⁻²R⁶⁴⁻², —C(═O)OR⁶⁶⁻², or —C(═O)NR⁶⁹⁻²R⁶¹⁰⁻²;

R⁶⁵, R⁶⁵⁻², R⁶³, R⁶³⁻², R⁶⁴, R⁶⁴⁻², R⁶⁶, R⁶⁶⁻², R⁶⁹, R⁶⁹⁻², R⁶¹⁰ and R⁶¹⁰⁻² are independently hydrogen or C₁₋₆ alkyl;

m is 0, 1 or 2;

R⁵ is independently C₁₋₆ alkyl;

R³¹ is —OR³¹, —SR³² or —NR³³R³⁴;

R³¹, R³² and R³⁴ are independently C₁₋₆ alkyl substituted with one or more R³¹⁻¹; R³³ is independently H, C₁₋₆ alkyl, or C₁₋₆ alkyl substituted with one or more R³¹⁻¹;

R³¹⁻¹ is independently C₃₄₀ cycloalkyl. “4-10 membered heterocycloalkyl containing 1-3 heteroatoms selected from one or more of O and N”, C₃₄₀ cycloalkyl substituted with one or more R^{d16}. “4-10 membered heterocycloalkyl containing 1-3 heteroatoms selected from one or more of O and N” substituted with one or more R^{d15}, —OR^{d}, —SR^{d1}, —NR^{e1}R^{e2} or —C(═O)NR^{e3}R^{e3};

R^{d15} and R^{d16} are independently C₁₋₆ alkyl, C₁₋₆ alkyl substituted with one or more R¹⁻⁸⁻¹, hydroxyl, C₁₋₆ alkoxy, halogen, —NR^{e5}R^{e6} or —C(═O)NR^{e7}R^{e8};

R^{d}, R^{d1}, R^{e1}, R^{e2}, R^{e3} and R^{e4} are independently hydrogen, C₁₋₆ alkyl, C₃₋₁₀ cycloalkyl, “4-10 membered heterocycloalkyl containing 1-3 heteroatoms selected from one or more of O and N”, or C₁₋₆ alkyl substituted with one or more R¹⁻³²;

R¹⁻⁸⁻¹ and R¹⁻⁸⁻² are independently cyano, halogen, hydroxyl, C₁₋₆ alkoxy, —C(═O)R^{e9}, —NR^{e10}R^{e11}, —C(═O)OR^{e12}, or —C(═O)NR^{e13}R^{e14};

R^{e5}, R^{e6}, R^{e7}, R^{e8}, R^{e9}, R^{e10}, R^{e11}, R^{e12}, R^{e13} and R^{e14} are independently hydrogen or C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring, a bridged ring or a spiro ring;

G is N, C or CH;

n is 0, 1, 2 or 3;

R⁴ is independently C₁₋₆ alkyl, C₁₋₆ alkyl substituted with one or more R³⁻¹, oxo. —C(═O)OR^{4a} or —C(═O)NR^{4b}R^{4c};

R⁴⁻¹ is independently halogen, cyano, hydroxyl, C₁₋₆ alkoxy, —NR⁴ⁱR^{4j}, —C(═O)OR^{4d} or —C(═O)NR^{4e}R^{4f}; R^{4d}, R^{4e}, R^{4f}, R⁴ⁱ and R^{4j} are independently hydrogen or C₁₋₆ alkyl;

R^{4a}, R^{4b} and R^{4c} are independently hydrogen or C₁₋₆ alkyl;

R² is CN, —C(═O)—C(R^{a})═C(R^{b})(R^{f}), —C(═O)—C≡CR^{f}, —S(═O)₂—C(R^{a})═C(R^{b})(R^{f}) or —S(═O)₂—C≡CR^{f};

R^{a} is independently hydrogen, deuterium, halogen or C₁₋₆ alkyl;

R^{b} and R^{f} are independently hydrogen, deuterium, C₁₋₆ alkyl, C₁₋₆ alkyl-C(═O)—, or C₁₋₆ alkyl substituted with one or more R^{b-1};

R^{b-1} is independently halogen, hydroxyl, C₁₋₆ alkoxy, or

R^{10j} and R^{10k} are independently hydrogen or C₁₋₆ alkyl, or R^{10j} and R^{10k} taken together with the N atom to which they are attached form “4-10 membered heterocycloalkyl containing 1-3 heteroatoms selected from one or more of O and N”.

In a certain embodiment, with regard to an oxygen-containing heterocyclic compound represented by formula I, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, some groups are as defined as follows, and the unmentioned group definitions are as described in any one of the embodiments of the present disclosure (this content is hereinafter referred to simply as “in a certain embodiment”). With regard to an oxygen-containing heterocyclic compound represented by formula I.

R¹ is C₆₋₂₀ aryl, “5-12 membered heteroaryl containing 1-4 heteroatoms selected from one or more of O, S and N”. C₆₋₂₀ aryl substituted with one or more R¹⁻⁶, or “5-12 membered heteroaryl containing 1-4 heteroatoms selected from one or more of O, S and N” substituted with one or more R¹⁻⁷;

R¹⁻⁶ and R¹⁻⁷ are independently halogen, hydroxyl, —C(═O)R⁶⁵, —NR⁶³R⁶⁴, —C(═O)NR⁶⁹R⁶¹⁰, —C(═O)OR⁶⁶. C₁₋₆ alkyl, C₁₋₆ alkoxy. C₃₋₁₀ cycloalkyl. “5-7 membered heterocycloalkyl containing 1 or 2 heteroatoms selected from one or more of O and N”. C₆₋₂₀ aryl, “5-7 membered heteroaryl containing 1 or 2 heteroatoms selected from one or more of O and N”. C₁₋₆ alkyl substituted with one or more R¹⁻⁶⁻¹. C₁₋₆ alkoxy substituted with one or more R¹⁻⁶⁻². C₃₋₁₀ cycloalkyl substituted with one or more R¹⁻⁶⁻³, “5-7 membered heterocycloalkyl containing 1 or 2 heteroatoms selected from one or more of O and N” substituted with one or more R¹⁻⁶⁻⁴. C₆₋₂₀ aryl substituted with one or more R¹⁻⁶⁻⁵, or “5-7 membered heteroaryl containing 1 or 2 heteroatoms selected from one or more of O and N” substituted with one or more R¹⁻⁶⁻⁶;

R¹⁻⁶⁻¹, R¹⁻⁶⁻², R¹⁻⁶⁻³, R¹⁻⁶⁻⁴, R¹⁻⁶⁻⁵ and R¹⁻⁶⁻⁶ are independently cyano, halogen, hydroxyl, C₁₋₆ alkoxy. C₁₋₆ alkyl, —C(═O)R⁶⁵⁻², —NR⁶³⁻²R⁶⁴⁻², —C(═O)OR⁶⁶⁻², or —C(═O)NR⁶⁹⁻²R⁶¹⁰⁻²;

R⁶⁵, R⁶⁵⁻², R⁶³, R⁶³⁻², R⁶⁴, R⁶⁴⁻², R⁶⁶, R⁶⁶⁻², R⁶⁹, R⁶⁹⁻², R⁶¹⁰ and R⁶¹⁰⁻² are independently hydrogen or C₁₋₆ alkyl;

m is 0, 1 or 2;

R⁵ is independently C₁₋₆ alkyl;

R³ is —OR³¹, —SR³² or —NR³³R³⁴;

R³¹, R³², R³³ and R³⁴ are independently C₁₋₆ alkyl substituted with one or more R³¹⁻¹;

R³¹⁻¹ is independently C₃₋₁₀ cycloalkyl. “4-10 membered heterocycloalkyl containing 1-3 heteroatoms selected from one or more of O and N”, C₃₋₁₀ cycloalkyl substituted with one or more R^{d16}, “4-10 membered heterocycloalkyl containing 1-3 heteroatoms selected from one or more of O and N” substituted with one or more R^{d15}, —OR^{d}, —SR^{d1}, —NR^{e1}R^{e2}, or —C(═O)NR^{e3}R^{e4};

R^{d15} and R^{d16} are independently C₁₋₆ alkyl, C₁₋₆ alkyl substituted with one or more hydroxyl, C₁₋₆ alkoxy, halogen, —NR^{e5}R^{e6} or —C(═O)NR^{e7}R^{e8};

R^{d}, R^{d1}, R^{e1}, R^{e2}, R^{e3} and R^{e4} are independently hydrogen, C₁₋₆ alkyl, C₃₋₁₀ cycloalkyl. “4-10 membered heterocycloalkyl containing 1-3 heteroatoms selected from one or more of 0 and N”, or C₁₋₆ alkyl substituted with one or more R¹⁻⁸⁻²;

R¹⁻⁸⁻¹ and R¹⁻⁸⁻² are independently cyano, halogen, hydroxyl, C₁₋₆ alkoxy, —C(═O)R^{e9}, —NR^{e10}R^{e11}, 13 C(═O)OR^{e12}, or —C(═O)NR^{e13}R^{e14};

R^{e5}, R^{e6}, R^{e7}, R^{e8}, R^{e9}, R^{e10}, R^{e11}, R^{e12}, R^{e13} and R^{e14} are independently hydrogen or C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring, a bridged ring or a Spiro ring;

G is N, C or CH;

n is 0, 1, 2 or 3;

R⁴ is independently C₁₋₆ alkyl, C₁₋₆ alkyl substituted with one or more R oxo. —C(═O)OR⁴³ or —C(═O)NR^{4b}R^{4c};

R⁴⁻¹ is independently halogen, cyano, hydroxyl, C₁₋₆ alkoxy, —NR⁴ⁱR^{4j}, —C(═O)OR^{4d} or —C(═O)NR^{4e}R^{4f}; R^{4d}, R^{4e}, R^{4f}, R⁴ⁱ and R^{4j} are independently hydrogen or C₁₋₆ alkyl;

R^{4a}, R^{4b} and R^{4c} are independently hydrogen or C₁₋₆ alkyl;

R² is —C(═O)—C(R^{a})═C(R^{b})(R^{f}), —C(═O)—C≡CR^{f}, —S(═O)₂C(R^{a})═C(R^{b})(R^{f}) or —S(═O)₂—C≡CR^{f};

R^{a} is independently hydrogen, deuterium, halogen or C₁₋₆ alkyl;

R^{b} and R^{f} are independently hydrogen, deuterium, C₁₋₆ alkyl, or C₁₋₆ alkyl substituted with one or more R^{b-1};

R^{b-1} is independently halogen, hydroxyl, C₁₋₆ alkoxy, or —NR^{10j}R^{10k};

R^{10j} and R^{10k} are independently hydrogen or C₁₋₆ alkyl, or R^{10j} and R^{10k} taken together with the N atom to which they are attached form “4-10 membered heterocycloalkyl containing 1-3 heteroatoms selected from one or more of O and N”.

In a certain embodiment:

R¹ is C₆₋₂₀ aryl, “5-12 membered heteroaryl containing 1-4 heteroatoms selected from one or more of O, S and N”. C₆₋₂₀ aryl substituted with one or more R¹⁻⁶, or “5-12 membered heteroaryl containing 1-4 heteroatoms selected from one or more of O, S and N” substituted with one or more R¹⁻⁷;

R¹⁻⁶ and R¹⁻⁷ are independently halogen, hydroxyl, C₁₋₆ alkyl, C₁₋₆ alkoxy. C₃₋₁₀ cycloalkyl, C₁₋₆ alkyl substituted with one or more R¹⁻⁶⁻¹, or C₁₋₆ alkoxy substituted with one or more R¹⁻⁶⁻²; R¹⁻⁶⁻¹ and R¹⁻⁶⁻² are independently halogen.

In a certain embodiment:

R¹ is C₆₋₂₀ aryl, “5-12 membered heteroaryl containing 1-4 heteroatoms selected from one or more of O, S and N”. C₆₋₂₀ aryl substituted with one or more R¹⁻⁶, or “5-12 membered heteroaryl containing 1-4 heteroatoms selected from one or more of O, S and N” substituted with one or more R¹⁻⁷; for example, C₆₋₂₀ aryl, “5-12 membered heteroaryl containing 1-4 heteroatoms selected from one or more of O, S and N”, or C₆₋₂₀ aryl substituted with one or more R¹⁻⁶;

R¹⁻⁶ and R¹⁻⁷ are independently halogen. C₁₋₆ alkyl, or C₁₋₆ alkyl substituted with one or more R¹⁻⁶⁻¹;

R¹⁻⁶⁻¹ is independently halogen.

In a certain embodiment:

R¹ is C₆₋₂₀ aryl, or C₆₋₂₀ aryl substituted with one or more R¹⁻⁶;

R¹⁻⁶ is independently halogen. C₁₋₆ alkyl, or C₁₋₆ alkyl substituted with one or more R¹⁻⁶⁻¹;

R¹⁻⁶⁻¹ is independently halogen.

In a certain embodiment:

m is 0.

In a certain embodiment:

R³ is —OR³¹, —SR³² or —NR³³R³⁴;

R³¹, R³² and R³⁴ are independently C₁₋₆ alkyl substituted with one or more R³¹⁻¹; R³³ is independently H, C₁₋₆ alkyl, or C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15} or —NR^{e1}R^{e2};

R^{d15} is independently C₁₋₆ alkyl, C₁₋₆ alkyl substituted with one or more R¹⁻⁸⁻¹, hydroxyl, C₁₋₆ alkoxy, halogen, —NR^{e5}R^{e6} or —C(═O)NR^{e7}R^{e8};

R¹⁻⁸⁻¹ is independently halogen; R^{e5}, R^{e6}, R^{e7} and R^{e8} are independently hydrogen or C₁₋₆ alkyl;

R^{e1} and R^{e2} are independently C₁₋₆ alkyl.

In a certain embodiment:

R³ is —SR³²;

R³² is C₁₋₆, alkyl substituted with one or more R³¹⁻¹;

R³¹⁻¹ is independently —NR^{e1}R^{e2};

R^{e1} and R^{e2} are independently C₁₋₆ alkyl.

In a certain embodiment:

R³ is —OR³¹ or —NR³³R³⁴;

R³¹, R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl, C₁₋₆ alkyl substituted with one or more R¹⁻⁸⁻¹, hydroxyl, C₁₋₆ alkoxy, halogen, —NR^{e5}R^{e6} or —C(═O)NR^{e7}R^{e8};

R¹⁻⁸⁻¹ is independently halogen; R^{e5}, R^{e6}, R^{e7} and R^{e8} are independently hydrogen or C₁₋₆ alkyl;

R^{e1} and R^{e2} are independently C₁₋₆ alkyl.

In a certain embodiment:

R³ is —NR³³R³⁴;

R³⁴ is independently C₁₋₆ alkyl substituted with one or more R³¹⁻¹; R³³ is independently H, C₁₋₆ alkyl, or C₁₋₆ alkyl substituted with one or more R³¹⁻¹; for example, H or C₁₋₆ alkyl;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl;

R^{e1} and R^{e2} are independently C₁₋₆ alkyl.

In a certain embodiment:

R³ is —OR³¹ or —NR³³R³⁴;

R³¹ and R³⁴ are independently C₁₋₆ alkyl substituted with one or more R³¹⁻¹; R″ is independently H, C₁₋₆ alkyl, or C₁₋₆ alkyl substituted with one or more R³¹⁻¹; for example, H or C₁₋₆ alkyl;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl or halogen;

R^{e1} and R^{e2} are independently C₁₋₆ alkyl.

In a certain embodiment:

R³ is —OR³¹R³⁴;

R³¹, R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl or halogen;

R^{e1} and R^{e2} are independently C₁₋₆ alkyl.

In a certain embodiment:

R³ is —NR³³R³⁴;

R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15};

R^{d15} is independently C₁₋₆ alkyl.

In a certain embodiment:

R³ is —OR³¹ or —NR³³R³⁴;

R³¹, R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{e1}, R^{e2} and R^{d15} are independently C₁₋₆ alkyl.

In a certain embodiment:

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring or a spiro ring; for example, a monocyclic ring;

G is N, C or CH.

In a certain embodiment:

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring;

G is N.

In a certain embodiment:

n is 0 or 1;

R⁴ is independently C₁₋₆ alkyl, or C₁₋₆ alkyl substituted with one or more R⁴⁻¹;

R⁴⁻¹ is independently hydroxyl, cyano, or —C(═O)NR^{4e}R^{4f}; R^{4e} and R^{4f} are independently hydrogen or C₁₋₆ alkyl; for example, hydrogen.

In a certain embodiment:

n is 0 or 1;

R⁴ is independently C₁₋₆ alkyl substituted with one or more R⁴⁻¹;

R⁴⁻¹ is independently —C(═O)NR^{4e}R^{4f}; R^{4e} and R^{4f} are independently hydrogen or C₁₋₆ alkyl; for example, hydrogen.

In a certain embodiment:

n is 0 or 1;

R⁴ is independently C₁₋₆ alkyl, or C₁₋₆ alkyl substituted with one or more R⁴⁻¹;

R⁴-1 is independently hydroxyl or cyano.

In a certain embodiment:

n is 0 or 1;

R⁴ is independently C₁₋₆ alkyl, or C₁₋₄ alkyl substituted with one or more R⁴⁻¹;

R⁴⁻¹ is independently hydroxyl.

In a certain embodiment:

n is 0 or 1;

R⁴ is independently C₁₋₆ alkyl, or C₁₋₆ alkyl substituted with one or more R⁴⁻¹.

R⁴⁻¹ is independently cyano.

In a certain embodiment:

R² is CN, —C(═O)—C(R^{a})═C(R^{b})(R^{f}), —C(═O)—C≡C-Me or, —S(═O)₂—C(R^{a})═C(R^{b})(R^{f});

R^{a} is independently hydrogen or halogen;

R^{b} and R^{f} are independently hydrogen, C₁₋₆ alkyl, C₁₋₆ alkyl-C(═O)—, or C₁₋₆ alkyl substituted with one or more R^{b-1};

R^{b-1} is independently —NR^{10j}R^{10k};

R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

In a certain embodiment:

R² is CN or —C(═O)—C(R^{a})═C(R^{b})(R^{f});

R^{a} is independently hydrogen;

R^{b} and R^{f} are independently hydrogen, C₁₋₆ alkyl-C(═O)—.

In a certain embodiment:

R² is —C(═O)—C(R^{a})═C(R^{b})(R^{f}), —C(═O)—C≡C-Me or —S(═O)₂—C(R^{a})≡C(R^{b})(R^{f})_{;}

R^{a} is independently hydrogen or halogen;

R^{b} and R^{f} are independently hydrogen, C₁₋₆ alkyl, or C₁₋₆ alkyl substituted with one or more R^{b-1};

R^{b-1} is independently halogen, hydroxyl, C₁₋₆ alkoxy, or —NR^{10j}R^{10k};

R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

In a certain embodiment:

R² is —C(═O)—C(R^{a})═C(R^{b})(R^{f}), —C(═O)—C≡C-Me or —S(═O)₂—C(R^{a})≡C(R^{b})(R^{f});

R³ is independently hydrogen or halogen;

R^{b} and R^{f} are independently hydrogen, C₁₋₆ alkyl, or C₁₋₆ alkyl substituted with one or more R^{b-1};

R^{b-1} is independently —NR^{10j}R^{10k};

R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

In a certain embodiment:

R¹ is C₆₋₂₀ aryl. “5-12 membered heteroaryl containing 1-4 heteroatoms selected from one or more of O, S and N”. C₆₋₂₀ aryl substituted with one or more R¹⁻⁶, or “5-12 membered heteroaryl containing 1-4 heteroatoms selected from one or more of O, S and N” substituted with one or more R¹⁻⁷;

R¹⁻⁶ and R¹⁻⁷ are independently halogen. C₁₋₆ alkyl, C₁₋₆ alkyl substituted with one or more R¹⁻⁶⁻¹; R¹⁻⁶⁻¹ is independently halogen;

m is 0;

R³ is —OR³¹, —SR³² or —NR³³R³⁴;

R³¹, R³² and R³⁴ are independently C₁₋₆ alkyl substituted with one or more R³¹⁻¹; R³³ is independently H, C₁₋₆ alkyl, or C₁₋₆ alkyl substituted with one or more R³¹⁻¹; for example, H or C₁₋₆ alkyl;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl or halogen;

R^{e1} and R^{e2} are independently C₁₋₆ alkyl;

ring Y is a 4-12 membered heterocyclic ring containing 1-4 N atoms; the heterocyclic rung is a saturated heterocyclic ring or a partly saturated heterocyclic ring; the heterocyclic ring is a monocyclic ring or a Spiro ring;

G is N, C or CH;

n is 0 or 1;

R¹ is independently C₁₋₆ alkyl, or C₁₋₆ alkyl substituted with one or more R⁴⁻¹;

R⁴⁻¹ is independently cyano, hydroxyl or —C(═O)NR^{4e}R^{4f}; R^{4e} and R^{4f} are independently hydrogen or C₁₋₆ alkyl;

R² is CN, —C(═O)—C(R^{a})═C(R^{b})(R^{f}), —C(═O)—C≡C-Me or, —S(═O)₂—C(R^{a})≡C(R^{b})(R^{f});

R^{a} is independently hydrogen or halogen;

R^{b} and R^{f} are independently hydrogen, C₁₋₆ alkyl, C₁₋₆ alkyl-C(═O)—, or C₁₋₆ alkyl substituted with one or more R^{b-1};

R^{b-1} is independently halogen, hydroxyl, C₁₋₆ alkoxy, or —NR^{10j}R^{10k};

R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

In a certain embodiment:

R¹ is C₆₋₂₀ aryl. “5-12 membered heteroaryl containing 1-4 heteroatoms selected from one or more of O, S and N”, C₆₋₂₀ aryl substituted with one or more R¹⁻⁶, or “5-membered heteroaryl containing 1-4 heteroatoms selected from one or more of O, S and N” substituted with one or more R¹⁻⁷;

R¹⁻⁶ and R¹⁻⁷ are independently halogen, hydroxyl, C₁₋₆ alkyl, C₁₋₆ alkoxy, C₃₋₁₀ cycloalkyl, C₁₋₆ alkyl substituted with one or more R¹⁻⁶⁻¹, or C₁₋₆ alkoxy substituted with one or more R¹⁻⁶⁻²; R¹⁻⁶⁻¹ and R¹⁻⁶⁻² are independently halogen;

m is 0;

R³ is —OR³¹ or —NR³³R³⁴;

R³¹, R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl, C₁₋₆ alkyl substituted with one or more R¹⁻⁸⁻¹, hydroxyl, C₁₋₆ alkoxy, halogen, —NR^{e5}R^{e6} or —C(═O)NR^{e7}R^{e8};

R¹⁻⁸⁻¹ is independently halogen; R^{e5}, R^{e6}, R^{e7} and R^{e8} are independently hydrogen or C₁₋₅ alkyl;

R^{e1} and R^{e2} are independently C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring or a spiro ring;

G is N, C or CH;

n is 0 or 1;

R⁴ is independently C₁₋₆ alkyl, or C₁₋₆ alkyl substituted with one or more R⁴⁻¹;

R⁴⁻¹ is independently cyano or hydroxyl;

R² is —C(═O)—C(R²)═C(R^{b})(R^{f}), —C(═O)—C≡C-Me or —S(═O)₂—C(R^{a})═C(R^{b})(R^{f});

R^{a} is independently hydrogen or halogen;

R^{b} and R^{f} are independently hydrogen, C₁₋₆ alkyl, or C₁₋₆ alkyl substituted with one or more R^{b-1};

R^{b-1} is independently halogen, hydroxyl, C₁₋₆ alkoxy, or —NR^{10j}R^{10k};

R^{10j} and R^{10k} are independently hydrogen or C, alkyl, or R^{10j} and R^{10k} taken together with the N atom to which they are attached form “4-6 membered heterocycloalkyl containing 1-3 heteroatoms selected from one or more of O and N”.

In a certain embodiment;

R² is —C(═O)—C(R^{a})═C(R^{b})(R^{f}): W is independently hydrogen or halogen; R^{b} and R^{f} are independently hydrogen or C₁₋₆ alkyl.

In a certain embodiment:

R¹ is C₆₋₂₀ aryl, “5-12 membered heteroaryl containing 1-4 heteroatoms selected from one or more of O, S and N”, C₆₋₂₀ aryl substituted with one or more R¹⁻⁶, or “5-12 membered heteroaryl containing 1-4 heteroatoms selected from one or more of O, S and N” substituted with one or more R¹⁻⁷;

R¹⁻⁶ and R¹⁻⁷ are independently halogen, hydroxyl, C₁₋₆ alkyl, C₁₋₆ alkoxy. C₃₋₁₀ cycloalkyl, C₁₋₆ alkyl substituted with one or more R¹⁻⁶⁻¹, or C₁₋₆ alkoxy substituted with one or more R¹⁻⁶⁻²; R¹⁻⁶⁻¹ and R¹⁻⁶⁻² are independently halogen;

m is 0;

R³ is —OR³¹ or —NR³³R³⁴;

R³¹, R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl, C₁₋₆ alkyl substituted with one or more R¹⁻⁸⁻¹, hydroxyl, C₁₋₆ alkoxy, halogen, —NR^{e5}R^{e6} or —C(═O)NR^{e7}R^{e8};

R¹⁻⁸⁻¹ is independently halogen; R^{e5}, R^{e6}, R^{e7} and R^{e8} are independently hydrogen or C₁₋₆ alkyl;

R^{e1} and R^{e2} are independently C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring or a Spiro ring;

G is N, C or CH;

n is 0 or 1;

R⁴ is independently C₁₋₆ alkyl, or C₁₋₆ alkyl substituted with one or more R⁴⁻¹;

R⁴⁻¹ is independently cyano;

R² is —C(═O)—C(R^{a})═C(R^{b})(R^{f}), —C(═O)—C≡C-Me or —S(═O)₂—C(R^{a})═C(R^{b})(R^{f});

R² is independently hydrogen or halogen;

R^{b} and R^{f} are independently hydrogen, C₁₋₆ alkyl, or C₁₋₆ alkyl substituted with one or more R^{b-1};

R^{b-1} is independently halogen, hydroxyl, C₁₋₆ alkoxy, or —NR¹⁶³R¹⁰ k;

R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

In a certain embodiment:

R¹ is C₆₋₂₀ aryl, “5-12 membered heteroaryl containing 1-4 heteroatoms selected from one or more of O, S and N”, C₆₋₂₀ aryl substituted with one or more R¹⁻⁶, or “5-12 membered heteroaryl containing 1-4 heteroatoms selected from one or more of O, S and N” substituted with one or more R¹⁻⁷; for example, C₆₋₂₀ aryl. “5-12 membered heteroaryl containing 1-4 heteroatoms selected from one or more of O, S and N”, or C₆₋₂₀ aryl substituted with one or more R¹⁻⁶;

R¹⁻⁶ is independently halogen, C₁₋₆ alkyl, or C₁₋₆ alkyl substituted with one or more R¹⁻⁶⁻¹;

R¹⁻⁶⁻¹ is independently halogen;

m is 0;

R³ is —OR³¹, —SR³² or —NR³³R³⁴;

R³¹, R³² and R³⁴ are independently C₁₋₆ alkyl substituted with one or more R³¹⁻¹; R³³ is independently H, C₁₋₆ alkyl, or C₁₋₆ alkyl substituted with one or more R³¹⁻¹; for example, H or C₁₋₆ alkyl;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl or halogen;

R^{e1} and R^{e2} are independently C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring;

G is N;

n is 0 or 1;

R⁴ is independently C₁₋₆ alkyl substituted with one or more R⁴⁻¹;

R⁴⁻¹ is independently cyano or —C(═O)NR^{4e}R^{4f}; R^{4e} and R^{4f} are independently hydrogen or C₁₋₆ alkyl;

R² is CN, —C(═O)—C(R^{a})═C(R^{b})(R^{f});

R^{a} is independently hydrogen or halogen;

R^{b} and R^{f} are independently hydrogen, C₁₋₆ alkyl, or C₁₋₆ alkyl-C(═O)—.

In a certain embodiment:

R¹ is C₆₋₂₀ aryl, or C₆₋₂₀ aryl substituted with one or more R¹⁻⁶;

R¹⁻⁶ is independently halogen, C₁₋₆ alkyl, or C₁₋₆ alkyl substituted with one or more R¹⁻⁶⁻¹ is independently halogen;

m is 0;

R³ is —OR³¹ or —NR³³R³⁴;

R³¹, R³³ and R³⁴ are independently C₁₋₆ alkyl substituted with one or more R³¹⁻¹;

R³¹⁻¹ is independently “5-7 membered heterocycloalkyl containing 1-3 heteroatoms selected from one or more of O and N” substituted with one or more R^{d15}, or —NR^{e1}R^{e2};

R^{d15} is independently C₁₋₆ alkyl or halogen;

R^{e1} and R^{e2} are independently C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring;

G is N;

n is 0 or 1;

R⁴ is independently C₁₋₆ alkyl, or C₁₋₆ alkyl substituted with one or more R⁴⁻¹;

R⁴⁻¹ is independently cyano;

R² is —C(═O)—C(R^{a})═C(R^{b})(R^{f});

R^{a} is independently hydrogen or halogen;

R^{b} and R^{f} are independently hydrogen or C₁₋₆ alkyl, or C₁₋₆ alkyl substituted with one or more R^{b-1};

R^{b-1} is independently —NR¹⁹¹R^{10k};

R¹⁰¹ and R^{10k} are independently hydrogen or C₁₋₆ alkyl, or R^{10j} and R^{10k} taken together with the N atom to which they are attached form “4-10 membered heterocycloalkyl containing 1-3 heteroatoms selected from one or more of O and N”.

In a certain embodiment:

R¹ is C₆₋₂₀ aryl, or C₆₋₂₀ aryl substituted with one or more R^{1′};

R¹⁻⁶ is independently halogen. C₁₋₆ alkyl, or C₁₋₆ alkyl substituted with one or more R¹⁻⁶⁻¹;

R¹⁻⁶⁻¹ is independently halogen;

m is 0;

R³ is —OR³¹ or —NR³³R³⁴;

R³¹, R³³ and R³⁴ are independently C₁₋₆ alkyl substituted with one or more R³¹⁻¹;

R³¹¹ is independently “5-7 membered heterocycloalkyl containing 1-3 heteroatoms selected from one or more of O and N” substituted with one or more R^{d15}, or —NR^{e1}R^{e2};

R^{e1}, R^{e2} and R^{d15} are independently C₁₋₆ alkyl;

ring Y is a 4-12 membered heterocyclic ring containing 1-4 N atoms; the heterocyclic ring is a saturated heterocyclic ring or a partly saturated heterocyclic ring; the heterocyclic ring is a monocyclic ring;

G is N;

n is 0 or 1;

R⁴ is independently C₁₋₆ alkyl, or C₁₋₆ alkyl substituted with one or more R⁴⁻¹;

R⁴⁻¹ is independently cyano;

R² is —C(═O)—C(R^{a})═C(R^{b})(R^{f});

R^{a} is independently hydrogen or halogen;

R^{b} and R^{f} are independently hydrogen or C₁₋₆ alkyl;

In a certain embodiment:

the oxygen-containing heterocyclic compound represented by formula I has a structure as follows:

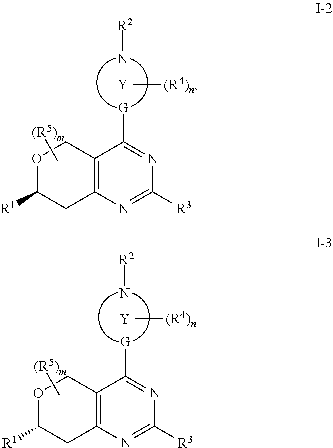

or “a mixture of

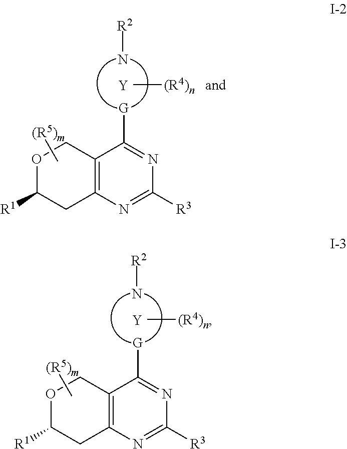

with a molar ratio of, for example, 1:1”.

In a certain embodiment: the oxygen-containing heterocyclic compound represented by formula I has a structure as follows:

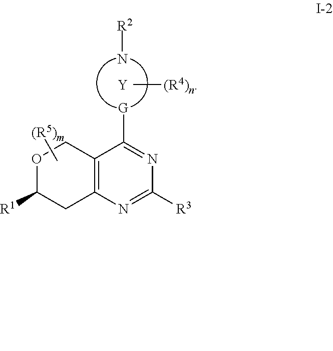

In a certain embodiment:

when R¹ is C₆₋₂₀ aryl, then the C₆₋₂₀ aryl may be phenyl or naphthyl, or may be phenyl or 1-naphthyl.

In a certain embodiment:

when R¹ is “5-12 membered heteroaryl containing 1-4 heteroatoms selected from one or more of O, S and N”, then the “5-12 membered heteroaryl containing 1-4 heteroatoms selected from one or more of O, S and N” may be “9-10 membered heteroaryl containing 1-4 heteroatoms selected from one or more of O, S and N”, or may be “9-10 membered heteroaryl containing 1 heteroatom selected from one of O, S and N”, or may be isoquinolyl, or may be

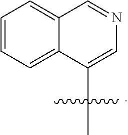 [SMILES: *c1cncc2ccccc12]

In a certain embodiment:

when R¹ is C₆₋₂₀ aryl substituted with one or more R¹⁻⁶, then the C₆₋₂₀ aryl may be phenyl or naphthyl, or may be phenyl or 1-naphthyl.

In a certain embodiment:

when R¹ is C₆₋₂₀ aryl substituted with one or more R¹⁻⁶, then the more R¹⁻⁶ may be two or three R¹⁻⁶.

In a certain embodiment:

when R¹⁻⁶ is independently halogen, then the halogen may be fluorine, chlorine, bromine or iodine, or may be fluorine or chlorine.

In a certain embodiment:

when R¹⁻⁶ is independently C₁₋₆ alkyl, then the C₁₋₆ alkyl may be C₁₋₄ alkyl, or may be methyl, ethyl, n-propyl, isopropyl, n-butyl, isobutyl, sec-butyl or tert-butyl, or may be methyl.

In a certain embodiment:

when R¹⁻⁶ is independently C₁₋₆ alkyl substituted with one or more R¹⁻⁶⁻¹, then the C₁₋₆ alkyl may be C₁₋₄ alkyl, or may be methyl, ethyl, n-propyl, isopropyl, n-butyl, isobutyl, sec-butyl or tert-butyl, or may be methyl.

In a certain embodiment;

when R¹⁻⁶ is independently C₁₋₆ alkyl substituted with one or more R¹⁻⁶⁻¹, then the more R¹⁻⁶⁻¹ may be two or three R¹⁶⁻¹.

In a certain embodiment:

when R¹⁻⁶⁻¹ is independently halogen, then the halogen may be fluorine, chlorine, bromine or iodine, or may be fluorine.

In a certain embodiment:

when R¹⁻⁶ is independently C₁₋₆ alkyl substituted with one or more R¹⁻⁶⁻¹, then the C₁₋₆ alkyl substituted with one or more R¹⁻⁶⁻¹ may be trifluoromethyl.

In a certain embodiment:

when R¹ is C₆₋₂₀ aryl substituted with one or more R¹⁻⁶, then the C₆₋₂₀ aryl substituted with one or more R¹⁻⁶ is

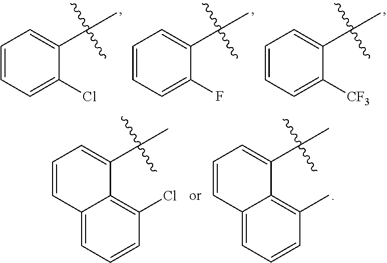

In a certain embodiment:

when R¹ is C₆₋₂₀ aryl substituted with one or more R¹⁻⁶, then the C₆₋₂₀ aryl substituted with one or more R¹⁻⁶ is

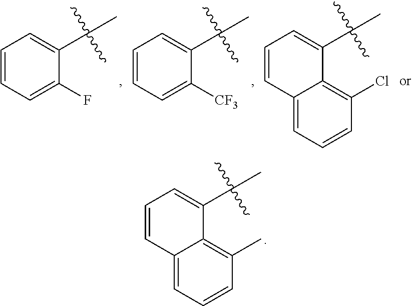 [SMILES: *c1ccccc1F ; *c1ccccc1C(F)(F)F ; *c1cccc2cccc(Cl)c12 ; *c1cccc2cccc(C)c12]

In a certain embodiment:

when R³³ is independently C₁₋₆ alkyl, then the C₁₋₆ alkyl may be C₁₋₄ alkyl, or may be methyl, ethyl, n-propyl, isopropyl, n-butyl, isobutyl, sec-butyl or tert-butyl, or may be methyl, ethyl, n-propyl or isopropyl.

In a certain embodiment:

when R³¹, R³³ and R³⁴ are independently C₁₋₆ alkyl substituted with one or more R³¹⁻¹, then the C₁₋₆ alkyl may be C₁₋₄ alkyl, or may be methyl, ethyl, n-propyl, isopropyl, n-butyl, isobutyl, sec-butyl or tert-butyl, or may be methyl, ethyl, n-propyl or isopropyl.

In a certain embodiment:

when R³¹, R³³ and R³⁴ are independently C₁₋₆ alkyl substituted with one or more R³¹⁻¹, then the more R³¹⁻¹ may be two or three R³¹⁻¹.

In a certain embodiment:

when R³¹⁻¹ is independently “4-10 membered heterocycloalkyl containing 1-3 heteroatoms selected from one or more of O and N” substituted with one or more R^{d15}, then the “4-10 membered heterocycloalkyl containing 1-3 heteroatoms selected from one or more of O and N” may be “5-7 membered heterocycloalkyl containing 1-3 heteroatoms selected from one or more of O and N”, or may be “5-7 membered heterocycloalkyl containing 1 heteroatom selected from one of O and N”, or may be tetrahydropyrrolyl, or more particularly, tetrahydropyrrole-2-yl.

In a certain embodiment:

when R³¹⁻¹ is independently “4-10 membered heterocycloalkyl containing 1-3 heteroatoms selected from one or more of O and N” substituted with one or more R^{d15}, then the more R^{d15} may be two or three R^{d15}.

In a certain embodiment:

when R^{d15} is independently C₁₋₆ alkyl, then the C₁₋₆ alkyl may be C₁₋₄ alkyl, or may be methyl, ethyl, n-propyl, isopropyl, n-butyl, isobutyl, sec-butyl or test-butyl, or may be methyl.

In a certain embodiment:

when R^{d15} is independently halogen, then the halogen may be fluorine, chlorine, bromine or iodine, or may be fluorine.

In a certain embodiment:

when R^{e1} and R^{e2} are independently C₁₋₆ alkyl, then the C₁₋₆ alkyl may be C₁₋₄ alkyl, or may be methyl, ethyl, n-propyl, isopropyl, n-butyl, isobutyl, sec-butyl or tert-butyl, or may be methyl or ethyl.

In a certain embodiment:

when R³¹, R³³ and R³⁴ are independently C₁₋₆ alkyl substituted with one or more R³¹⁻¹, then the C₁₋₆ alkyl substituted with one or more R³¹⁻¹ may be

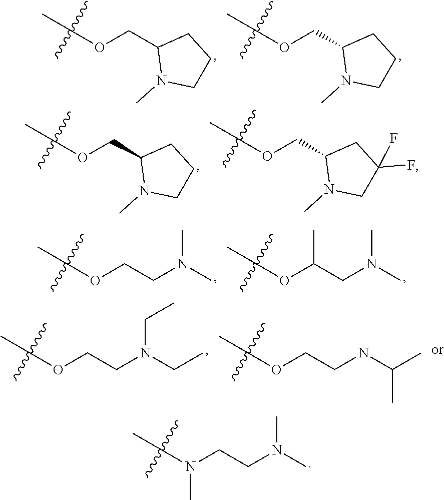

In a certain embodiment:

when ring Y is a 4-12 membered heterocyclic ring containing 1-4 N atoms, then the 4-12 membered heterocyclic ring containing 1-4 N atoms may be a 6-9 membered heterocyclic ring containing 1-2 N atoms or may be

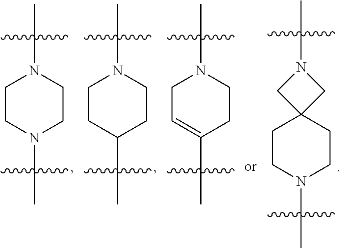

the upper end of which is connected to R².

In a certain embodiment:

the oxygen-containing heterocyclic compound represented by formula I has a structure as follows:

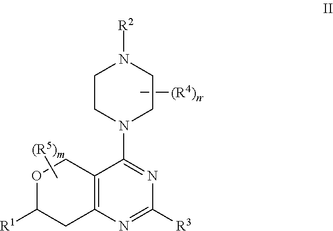

In a certain embodiment:

when R⁴ is independently C₁₋₆ alkyl, then the C₁₋₆ alkyl may be C₁₋₄ alkyl, or may be methyl, ethyl, n-propyl, isopropyl, n-butyl, isobutyl, sec-butyl or tert-butyl, or may be methyl.

In a certain embodiment:

when R⁴ is independently C₁₋₆ alkyl substituted with one or more R, then the Cm alkyl may be C₁₋₄ alkyl, or may be methyl, ethyl, n-propyl, isopropyl, n-butyl, isobutyl, sec-butyl or tert-butyl, or may be methyl.

In a certain embodiment:

when R⁴ is independently C₁₋₆ alkyl substituted with one or more R⁴⁻¹, then the more R⁴⁻¹ may be two or three R⁴⁻¹.

In a certain embodiment:

when R⁴ is independently C₁₋₆ alkyl substituted with one or more R⁴⁻¹, then the C₁₋₆ alkyl substituted with one or more R⁴⁻¹ may be hydroxymethyl, cyanomethyl or

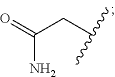 [SMILES: *CC(N)=O]

for example, cyanomethyl or

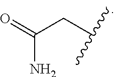 [SMILES: *CC(N)=O]

In a certain embodiment:

when R⁴ is independently C₁₋₆ alkyl substituted with one or more R⁴⁻¹, then the C₁₋₆ alkyl substituted with one or more R⁴⁻¹ may be

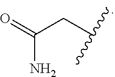 [SMILES: *CC(N)=O]

In a certain embodiment:

when R⁴ is independently C₁₋₆ alkyl substituted with one or more R⁴⁻¹, then the Cm alkyl substituted with one or more R⁴⁻¹ may be hydroxymethyl or cyanomethyl.

In a certain embodiment:

when R⁴ is independently C₁₋₆ alkyl substituted with one or more R⁴⁻¹ then the C₁₋₆ alkyl substituted with one or more R⁴⁻¹ may be hydroxymethyl.

In a certain embodiment:

when R⁴ is independently C₁₋₆ alkyl substituted with one or more R⁴⁻¹, then the C₁₋₆ alkyl substituted with one or more R⁴⁻¹ may be cyanomethyl.

In a certain embodiment:

when R^{a} is independently halogen, then the halogen may be fluorine, chlorine, bromine or iodine, or may be fluorine.

In a certain embodiment:

when R^{b} and R^{f} are independently C₁₋₆ alkyl, then the C₁₋₆ alkyl may be C₁₋₄ alkyl, or may be methyl, ethyl, n-propyl, isopropyl, n-butyl, isobutyl, sec-butyl or tert-butyl, or may be methyl.

In a certain embodiment:

when R^{b} and R^{f} are independently C₁₋₆ alkyl-C(═O)—, then the C₁₋₆ alkyl in the C₁₋₆ alkyl-C(═O)— may be C₁₋₄ alkyl, or may be methyl, ethyl, n-propyl, isopropyl, n-butyl, isobutyl, sec-butyl or tert-butyl, or may be methyl.

In a certain embodiment:

when R^{b} and R^{f} are independently C₁₋₆ alkyl substituted with one or more R, then the C₁₋₆ alkyl may be C₁₋₄ alkyl, or may be methyl, ethyl, n-propyl, isopropyl, n-butyl, isobutyl, sec-butyl or tert-butyl, or may be methyl.

In a certain embodiment:

when R^{b} and R^{f} are independently C₁₋₆ alkyl substituted with one or more R^{b-1}, then the more R^{b-1} may be two or three R^{b-1}.

In a certain embodiment:

when R^{10j} and R^{10k} are independently C₁₋₆ alkyl, then the C₁₋₆ alkyl may be C₁₋₄ alkyl, or may be methyl, ethyl, n-propyl, isopropyl, n-butyl, isobutyl, sec-butyl or tert-butyl, or may be methyl.

In a certain embodiment:

when R^{10j} and R^{10k} taken together with the N atom to which they are attached form “4-10 membered heterocycloalkyl containing 1-3 heteroatoms selected from one or more of O and N”, then the “4-10 membered heterocycloalkyl containing 1-3 heteroatoms selected from one or more of O and N” may be “5-6 membered heterocycloalkyl containing 2 heteroatoms selected from O and N”, or may be

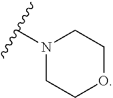 [SMILES: *N1CCOCC1]

In a certain embodiment:

the R² may be CN.

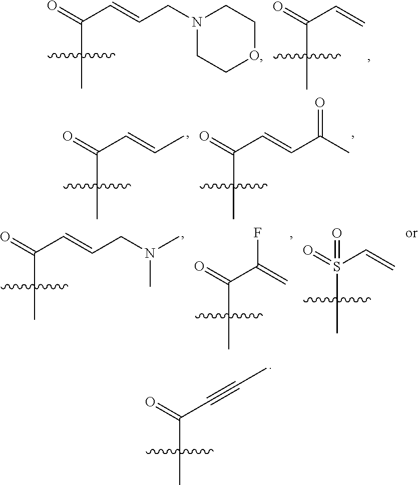

In a certain embodiment:

the R² may be CN.

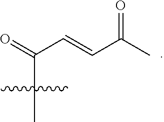 [SMILES: *C(=O)/C=C/C(C)=O]

In a certain embodiment:

the R² may be

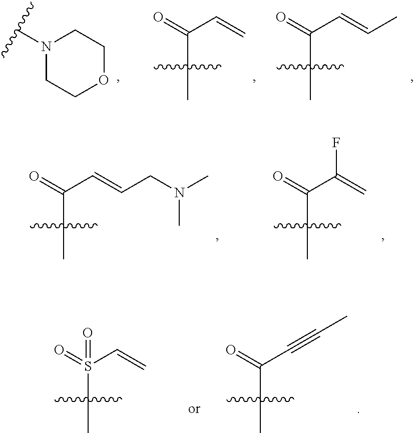

In a certain embodiment:

the R² may be

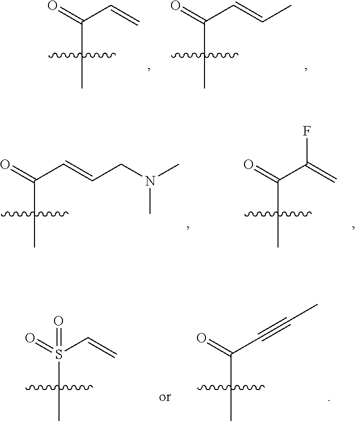

In a certain embodiment, the oxygen-containing heterocyclic compound represented by formula I has any one of the following structures;

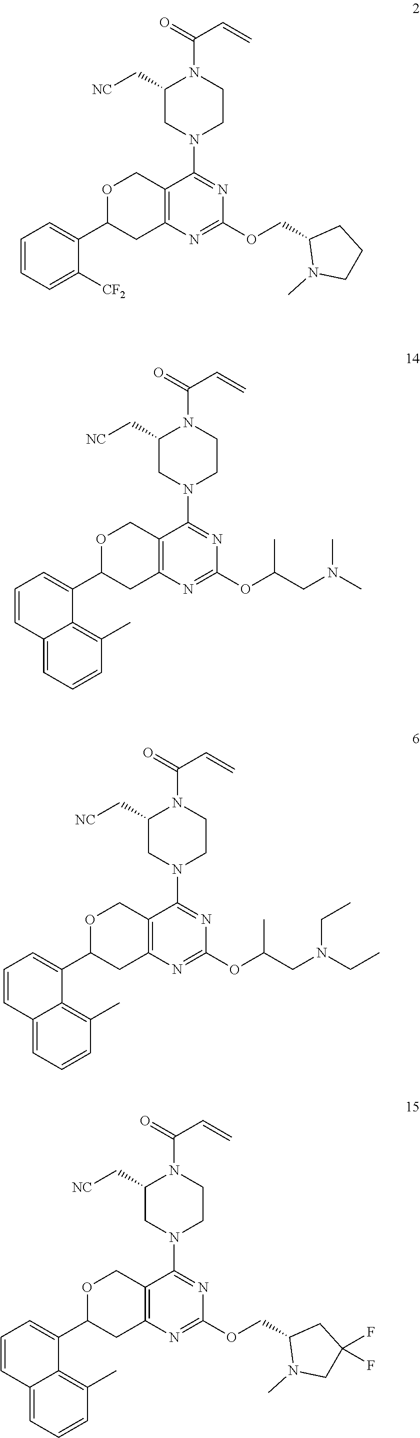

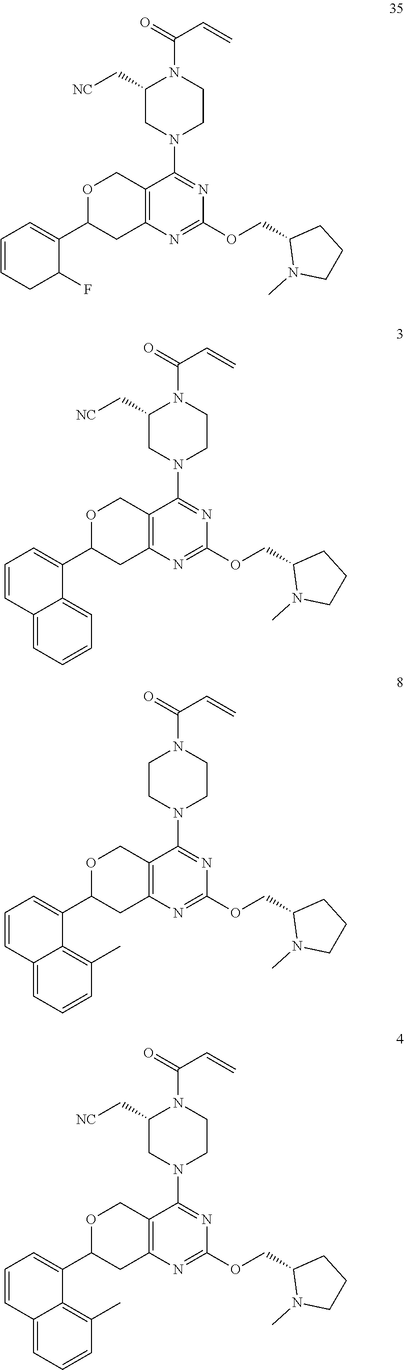 [SMILES: C=CC(=O)N1CCN(c2nc(OC[C@@H]3CCCN3C)nc3c2COC(C2=CC=CCC2F)C3)C[C@@H]1CC#N ; C=CC(=O)N1CCN(c2nc(OC[C@@H]3CCCN3C)nc3c2COC(c2cccc4ccccc24)C3)C[C@@H]1CC#N ; C=CC(=O)N1CCN(c2nc(OC[C@@H]3CCCN3C)nc3c2COC(c2cccc4cccc(C)c24)C3)CC1 ; C=CC(=O)N1CCN(c2nc(OC[C@@H]3CCCN3C)nc3c2COC(c2cccc4cccc(C)c24)C3)C[C@@H]1CC#N]

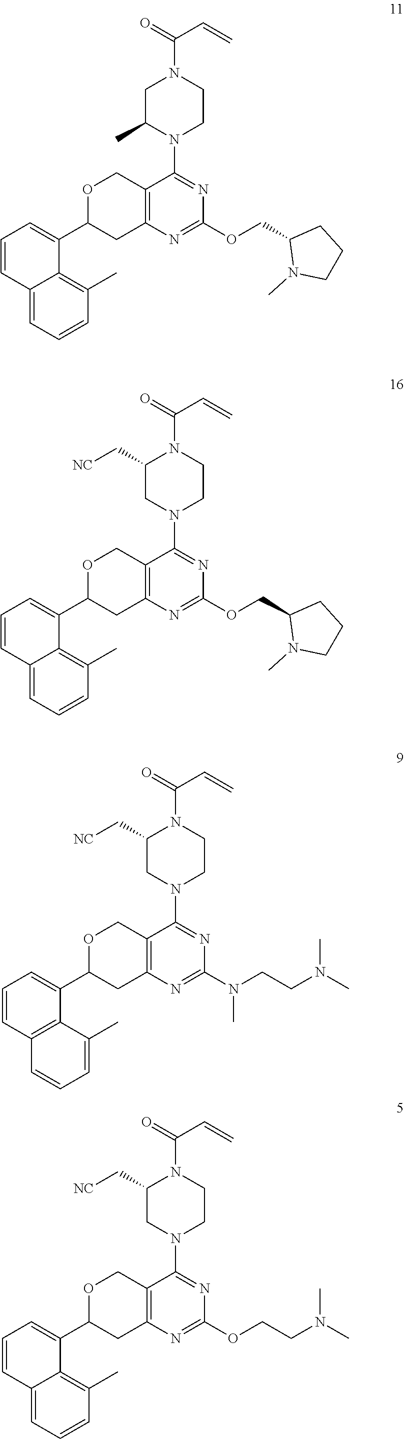 [SMILES: C=CC(=O)N1CCN(c2nc(OC[C@@H]3CCCN3C)nc3c2COC(c2cccc4cccc(C)c24)C3)[C@@H](C)C1 ; C=CC(=O)N1CCN(c2nc(OC[C@H]3CCCN3C)nc3c2COC(c2cccc4cccc(C)c24)C3)C[C@@H]1CC#N ; C=CC(=O)N1CCN(c2nc(N(C)CCN(C)C)nc3c2COC(c2cccc4cccc(C)c24)C3)C[C@@H]1CC#N ; C=CC(=O)N1CCN(c2nc(OCCN(C)C)nc3c2COC(c2cccc4cccc(C)c24)C3)C[C@@H]1CC#N]

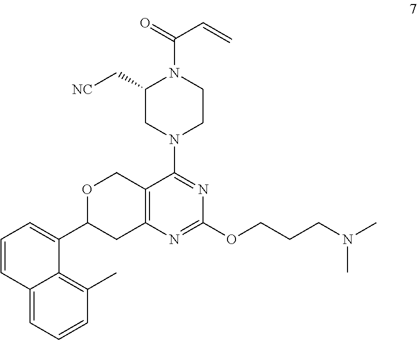 [SMILES: C=CC(=O)N1CCN(c2nc(OCCCN(C)C)nc3c2COC(c2cccc4cccc(C)c24)C3)C[C@@H]1CC#N]

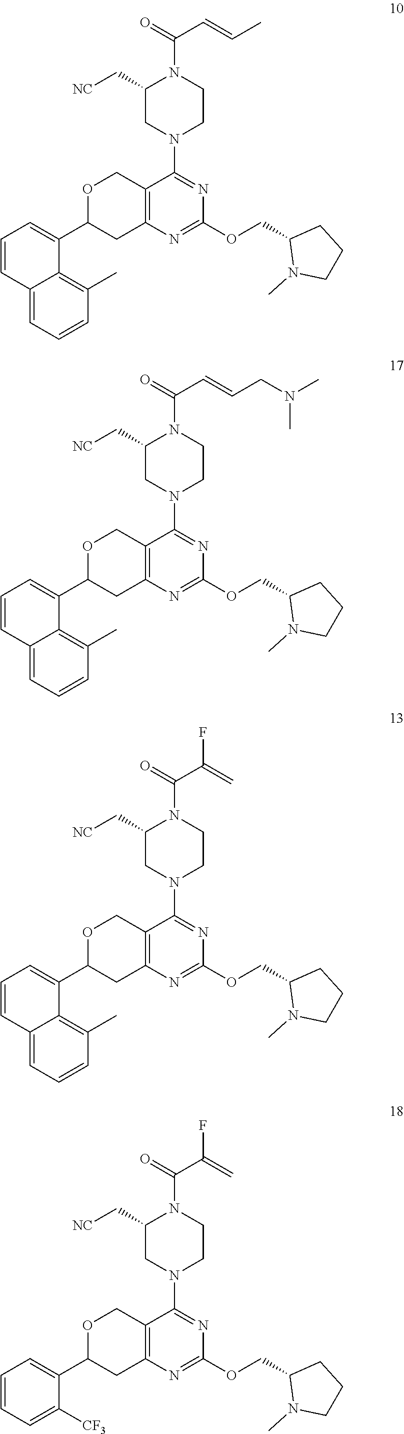 [SMILES: C/C=C/C(=O)N1CCN(c2nc(OC[C@@H]3CCCN3C)nc3c2COC(c2cccc4cccc(C)c24)C3)C[C@@H]1CC#N ; Cc1cccc2cccc(C3Cc4nc(OC[C@@H]5CCCN5C)nc(N5CCN(C(=O)/C=C/CN(C)C)[C@@H](CC#N)C5)c4CO3)c12 ; C=C(F)C(=O)N1CCN(c2nc(OC[C@@H]3CCCN3C)nc3c2COC(c2cccc4cccc(C)c24)C3)C[C@@H]1CC#N ; C=C(F)C(=O)N1CCN(c2nc(OC[C@@H]3CCCN3C)nc3c2COC(c2ccccc2C(F)(F)F)C3)C[C@@H]1CC#N]

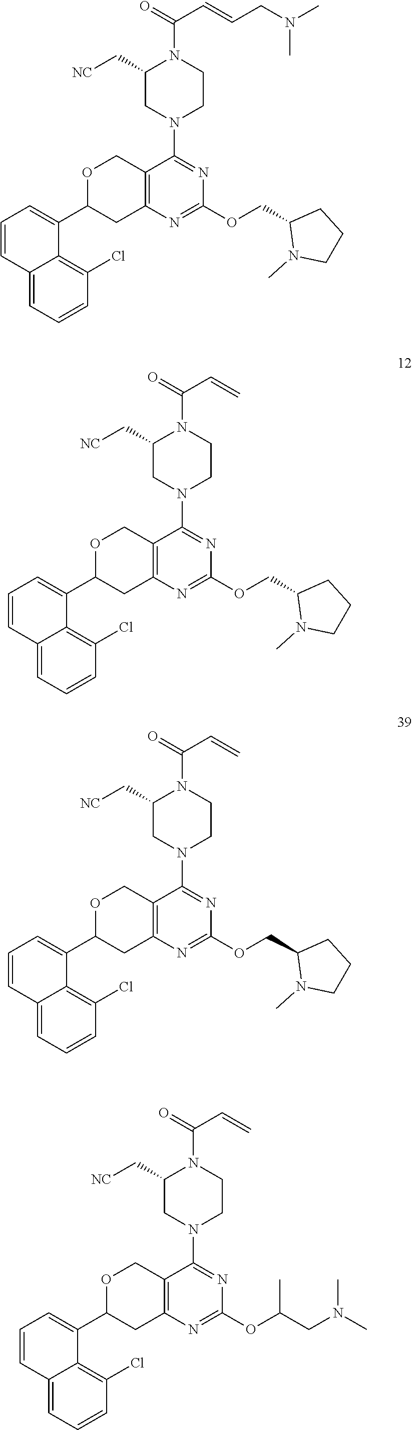 [SMILES: CN(C)C/C=C/C(=O)N1CCN(c2nc(OC[C@@H]3CCCN3C)nc3c2COC(c2cccc4cccc(Cl)c24)C3)C[C@@H]1CC#N ; C=CC(=O)N1CCN(c2nc(OC[C@@H]3CCCN3C)nc3c2COC(c2cccc4cccc(Cl)c24)C3)C[C@@H]1CC#N ; C=CC(=O)N1CCN(c2nc(OC[C@H]3CCCN3C)nc3c2COC(c2cccc4cccc(Cl)c24)C3)C[C@@H]1CC#N ; C=CC(=O)N1CCN(c2nc(OC(C)CN(C)C)nc3c2COC(c2cccc4cccc(Cl)c24)C3)C[C@@H]1CC#N]

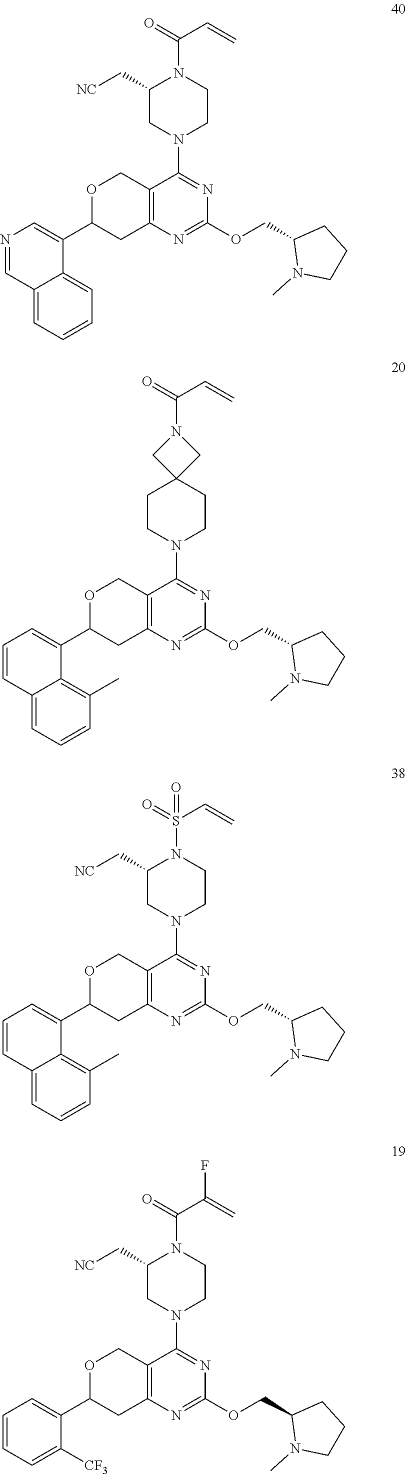 [SMILES: C=CC(=O)N1CCN(c2nc(OC[C@@H]3CCCN3C)nc3c2COC(c2cncc4ccccc24)C3)C[C@@H]1CC#N ; C=CC(=O)N1CC2(CCN(c3nc(OC[C@@H]4CCCN4C)nc4c3COC(c3cccc5cccc(C)c35)C4)CC2)C1 ; C=CS(=O)(=O)N1CCN(c2nc(OC[C@@H]3CCCN3C)nc3c2COC(c2cccc4cccc(C)c24)C3)C[C@@H]1CC#N ; C=C(F)C(=O)N1CCN(c2nc(OC[C@H]3CCCN3C)nc3c2COC(c2ccccc2C(F)(F)F)C3)C[C@@H]1CC#N]

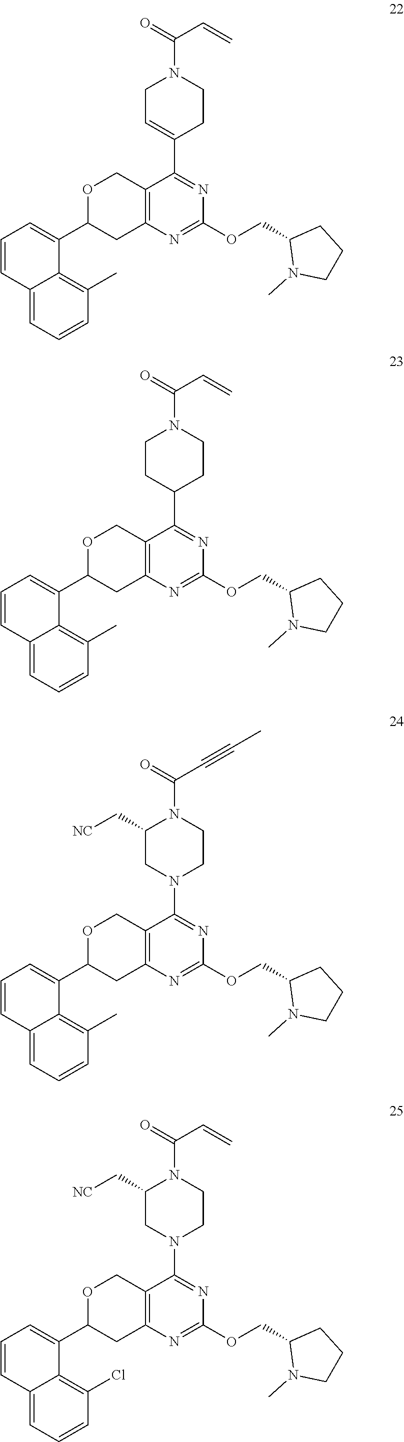 [SMILES: C=CC(=O)N1CC=C(c2nc(OC[C@@H]3CCCN3C)nc3c2COC(c2cccc4cccc(C)c24)C3)CC1 ; C=CC(=O)N1CCC(c2nc(OC[C@@H]3CCCN3C)nc3c2COC(c2cccc4cccc(C)c24)C3)CC1 ; CC#CC(=O)N1CCN(c2nc(OC[C@@H]3CCCN3C)nc3c2COC(c2cccc4cccc(C)c24)C3)C[C@@H]1CC#N ; C=CC(=O)N1CCN(c2nc(OC[C@@H]3CCCN3C)nc3c2COC(c2cccc4cccc(Cl)c24)C3)C[C@@H]1CC#N]

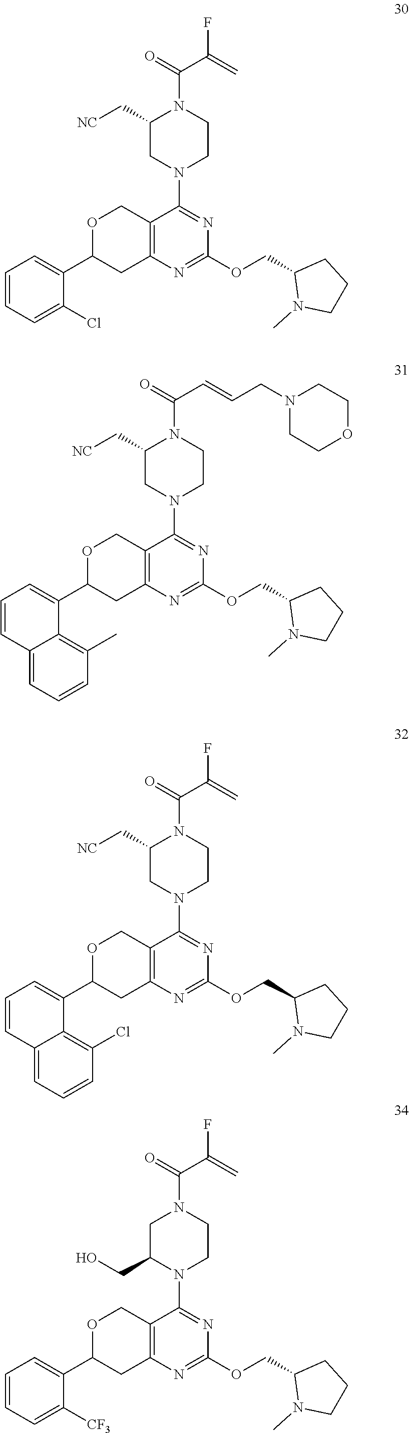 [SMILES: C=C(F)C(=O)N1CCN(c2nc(OC[C@@H]3CCCN3C)nc3c2COC(c2ccccc2Cl)C3)C[C@@H]1CC#N ; Cc1cccc2cccc(C3Cc4nc(OC[C@@H]5CCCN5C)nc(N5CCN(C(=O)/C=C/CN6CCOCC6)[C@@H](CC#N)C5)c4CO3)c12 ; C=C(F)C(=O)N1CCN(c2nc(OC[C@H]3CCCN3C)nc3c2COC(c2cccc4cccc(Cl)c24)C3)C[C@@H]1CC#N ; C=C(F)C(=O)N1CCN(c2nc(OC[C@@H]3CCCN3C)nc3c2COC(c2ccccc2C(F)(F)F)C3)[C@@H](CO)C1]

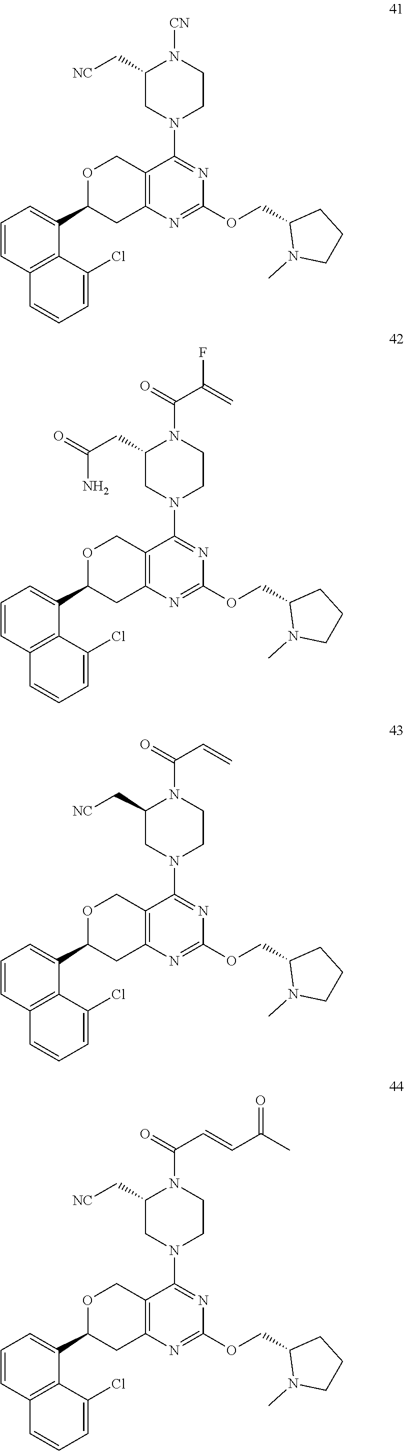 [SMILES: CN1CCC[C@H]1COc1nc2c(c(N3CCN(C#N)[C@@H](CC#N)C3)n1)CO[C@H](c1cccc3cccc(Cl)c13)C2 ; C=C(F)C(=O)N1CCN(c2nc(OC[C@@H]3CCCN3C)nc3c2CO[C@H](c2cccc4cccc(Cl)c24)C3)C[C@@H]1CC(N)=O ; C=CC(=O)N1CCN(c2nc(OC[C@@H]3CCCN3C)nc3c2CO[C@H](c2cccc4cccc(Cl)c24)C3)C[C@H]1CC#N ; CC(=O)/C=C/C(=O)N1CCN(c2nc(OC[C@@H]3CCCN3C)nc3c2CO[C@H](c2cccc4cccc(Cl)c24)C3)C[C@@H]1CC#N]

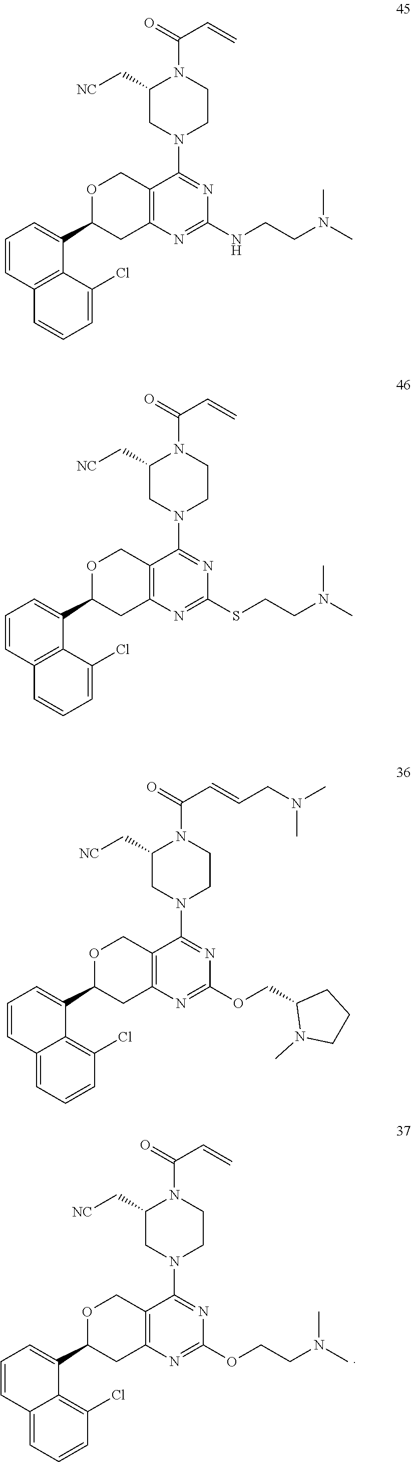 [SMILES: C=CC(=O)N1CCN(c2nc(NCCN(C)C)nc3c2CO[C@H](c2cccc4cccc(Cl)c24)C3)C[C@@H]1CC#N ; C=CC(=O)N1CCN(c2nc(SCCN(C)C)nc3c2CO[C@H](c2cccc4cccc(Cl)c24)C3)C[C@@H]1CC#N ; CN(C)C/C=C/C(=O)N1CCN(c2nc(OC[C@@H]3CCCN3C)nc3c2CO[C@H](c2cccc4cccc(Cl)c24)C3)C[C@@H]1CC#N ; C=CC(=O)N1CCN(c2nc(OCCN(C)C)nc3c2CO[C@H](c2cccc4cccc(Cl)c24)C3)C[C@@H]1CC#N]

In a certain embodiment, the oxygen-containing heterocyclic compound represented by formula I is any one of the following compounds:

compound

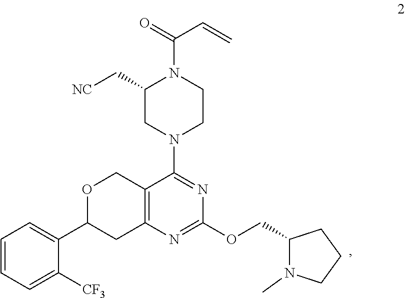 [SMILES: C=CC(=O)N1CCN(c2nc(OC[C@@H]3CCCN3C)nc3c2COC(c2ccccc2C(F)(F)F)C3)C[C@@H]1CC#N]

which has a retention time of 0.92 min under the following conditions: equipment: SFC Method Station (Thar, Waters): chromatographic column: OD-H 4.6*100 mm, 5 μm (Daicel): column temperature: 40° C.: mobile phase; CO₂/MeOH (0.1% TEA)=65/35: flow rate: 4.0 ml min: wavelength: 254 nm: back pressure; 120 bar;

compound

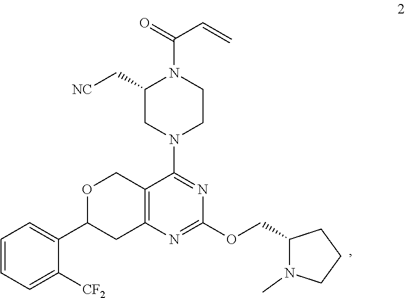

which has a retention tune of 2.74 min under the following conditions: equipment: SFC Method Station (Thar. Waters): chromatographic column: OD-H 4.6*100 mm, 5 μm (Daicel): column temperature: 40° C.: mobile phase; CO₂/MeOH (0.1% TEA)=65/35; flow rate: 4.0 nil/min: wavelength: 254 nm: back pressure; 120 bar;

compound

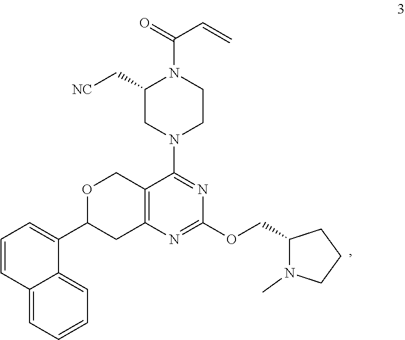 [SMILES: C=CC(=O)N1CCN(c2nc(OC[C@@H]3CCCN3C)nc3c2COC(c2cccc4ccccc24)C3)C[C@@H]1CC#N]

which has a retention time of 0.97 min under the following conditions: equipment: SFC Method Station (Thar. Waters): chromatographic column: AD-H 4.6*100 mm, 5 μm (Daicel): column temperature: 40° C.; mobile phase; CO₂/ETOH (0.5% TEA)=55/45: flow rate: 4.0 ml/min: wavelength: 254 nm: back pressure; 120 bar;

compound

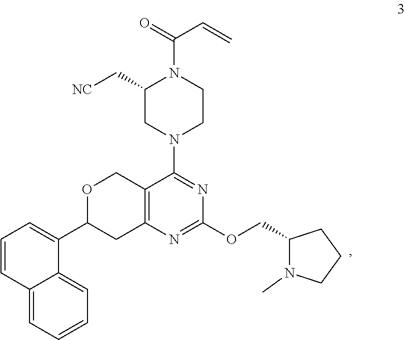 [SMILES: C=CC(=O)N1CCN(c2nc(OC[C@@H]3CCCN3C)nc3c2COC(c2cccc4ccccc24)C3)C[C@@H]1CC#N]

which has a retention time of 2.40 min under the following conditions: equipment: SFC Method Station (Thar. Waters): chromatographic column: AD-H 4.6*100 mm, 5 μm (Daicel): column temperature: 40° C.: mobile phase; CO₂/ETOH (0.5% TEA)=55/45: flow rate: 4.0 wavelength: 254 nm: back pressure; 120 bar;

compound

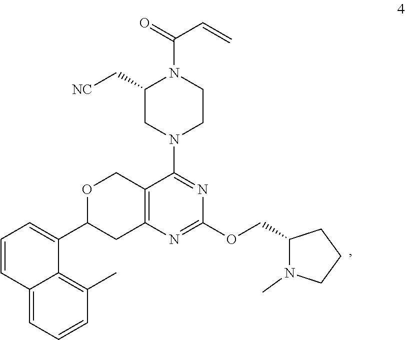 [SMILES: C=CC(=O)N1CCN(c2nc(OC[C@@H]3CCCN3C)nc3c2COC(c2cccc4cccc(C)c24)C3)C[C@@H]1CC#N]

which has a retention time of 0.97 min under the following conditions: equipment: SFC Method Station (Thar. Waters): chromatographic column: OJ-H 4.6*100 mm, 5 μm (Daicel): column temperature: 40° C.: mobile phase; CO₂/Methanol (0.1% TEA)=60/40; flow rate: 4.0 ml/min; wavelength: 254 inn: back pressure: 120 bar;

compound

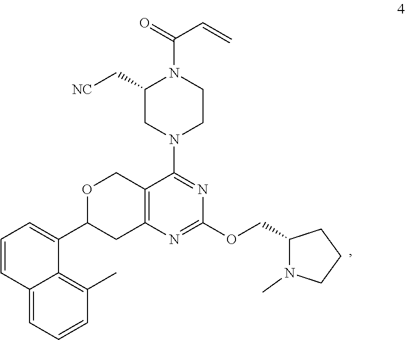 [SMILES: C=CC(=O)N1CCN(c2nc(OC[C@@H]3CCCN3C)nc3c2COC(c2cccc4cccc(C)c24)C3)C[C@@H]1CC#N]

which has a retention time of 1.94 min under the following conditions: equipment: SFC Method Station (Thar. Waters): chromatographic column: OJ-H 4.6*100 mm, 5 μm (Daicel): column temperature: 40° C.: mobile phase; CO₂/Methanol (0.1% TEA)=60/40: flow rate: 4.0 ml/min: wavelength: 254 nm: back pressure: 120 bar;

compound

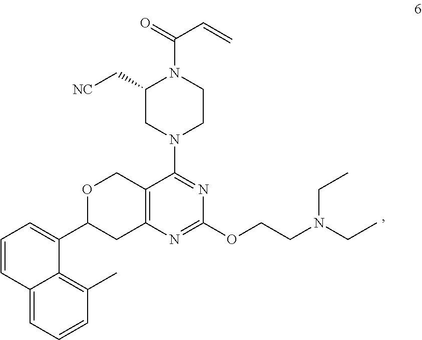 [SMILES: C=CC(=O)N1CCN(c2nc(OCCN(CC)CC)nc3c2COC(c2cccc4cccc(C)c24)C3)C[C@@H]1CC#N]

which has a retention time of 1.22 min under the following conditions: equipment: SFC Method Station (Thar. Waters): chromatographic column: CHIRALCEL OJ-H 4.6*100 mm, 5 μm (Daicel): column temperature: 40° C.; mobile phase: CO₂/MeOH (0.1% TEA)=65135; flow rate: 1.0 ml/min: wavelength: 214 nm; back pressure: 120 bar;

compound

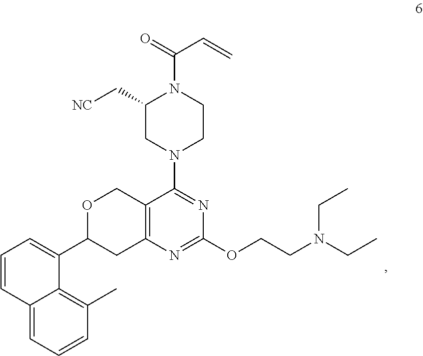 [SMILES: C=CC(=O)N1CCN(c2nc(OCCN(CC)CC)nc3c2COC(c2cccc4cccc(C)c24)C3)C[C@@H]1CC#N]

which has a retention time of 2.67 min under the following conditions: equipment: SFC Method Station (Thar. Waters); chromatographic column: CHIRALCEL OJ-H 4.6*100 mm, 5 μm (Daicel): column temperature: 40° C.; mobile phase: CO₂/MeOH (0.1% TEA)=65/35: flow rate: 1.0 ml/min: wavelength: 214 nm; back pressure: 120 bar;

compound

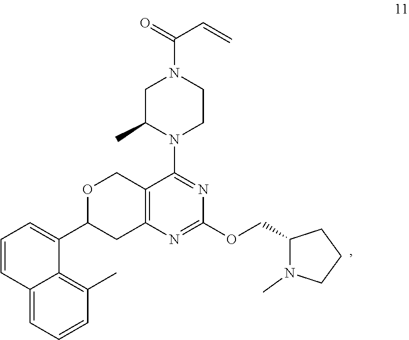 [SMILES: C=CC(=O)N1CCN(c2nc(OC[C@@H]3CCCN3C)nc3c2COC(c2cccc4cccc(C)c24)C3)[C@@H](C)C1]

which has a retention time of 3.26 min under the following conditions: instrument: SFC Method Station (Thar. Waters): chromatographic column: R,R-WHELK-O1 4.6*100 mm, 5 μm (REGIS): column temperature: 40° C.: mobile phase: CO₂/(MeOH/CAN=3:2 (0.1% TEA))=55/45: flow rate: 4.0 ml/min; wavelength; 254 nm: back pressure: 120 bar;

compound

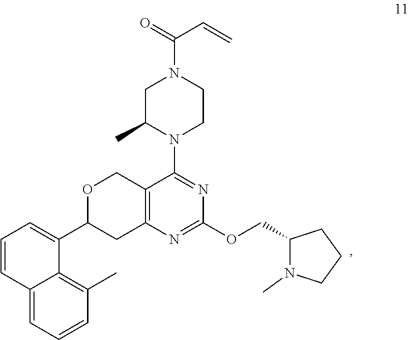 [SMILES: C=CC(=O)N1CCN(c2nc(OC[C@@H]3CCCN3C)nc3c2COC(c2cccc4cccc(C)c24)C3)[C@@H](C)C1]

which has a retention time of 4.16 min under the following conditions: instrument: SFC Method Station (Thar. Waters): chromatographic column: R,R-WHELK-O1 4.6*100 mm, 5 μm (REGIS): column temperature: 40° C.: mobile phase: CO₂/(MeOH/CAN=3:2 (0.1% TEA))=55/45: flow rate: 4.0 ml/min: wavelength; 254 null: back pressure: 120 bar;

compound

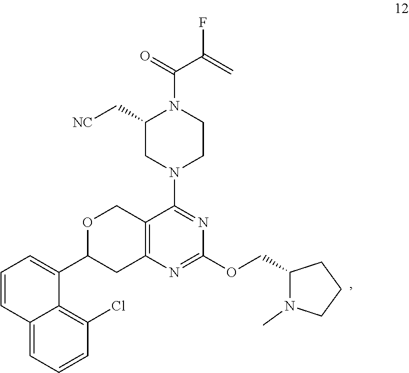 [SMILES: C=C(F)C(=O)N1CCN(c2nc(OC[C@@H]3CCCN3C)nc3c2COC(c2cccc4cccc(Cl)c24)C3)C[C@@H]1CC#N]

which has a retention time of 1.36 min under the following conditions: instrument: SFC Method Station (Thar. Waters): chromatographic column: OJ-H 4.6*100 mm, 5 μm (Daicel): column temperature: 40° C.: mobile phase; CO₂/MeOH (0.1% TEA)=60/40: flow rate: 4.0 wavelength: 254 nm: back pressure; 120 bar;

compound

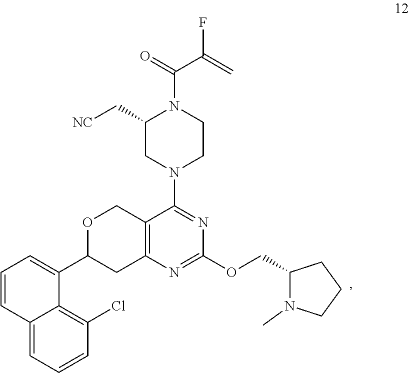 [SMILES: C=C(F)C(=O)N1CCN(c2nc(OC[C@@H]3CCCN3C)nc3c2COC(c2cccc4cccc(Cl)c24)C3)C[C@@H]1CC#N]

which has a retention time of 2.77 min under the following conditions: instrument: SFC Method Station (Thar. Waters): chromatographic column: OJ-H 4.6*100 mm, 5 μm (Daicel): column temperature: 40° C.: mobile phase; CO₂/MeOH (0.1% TEA)=60/40: flow rate: 4.0 ml/min; wavelength: 254 nm: back pressure; 120 bar;

compound

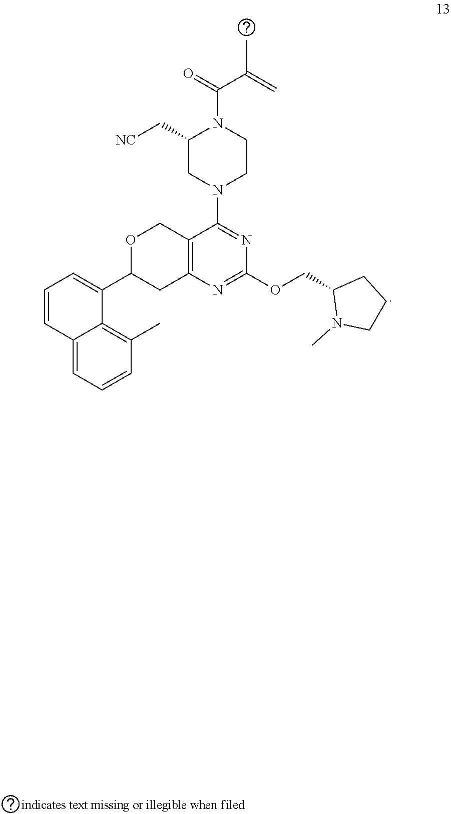 [SMILES: C=C(C)C(=O)N1CCN(c2nc(OC[C@@H]3CCCN3C)nc3c2COC(c2cccc4cccc(C)c24)C3)C[C@@H]1CC#N]

which has a retention time of 1.17 min under the following conditions: instrument: SFC Method Station (Thar. Waters): chromatographic column: OJ-H 4.6*100 mm, 5 μm (Daicel): column temperature: 40° C.: mobile phase; CO₂/MeOH (0.1% TEA)=60/40: flow rate: 4.0 ml/min: wavelength: 254 mm: back pressure; 120 bar;

compound

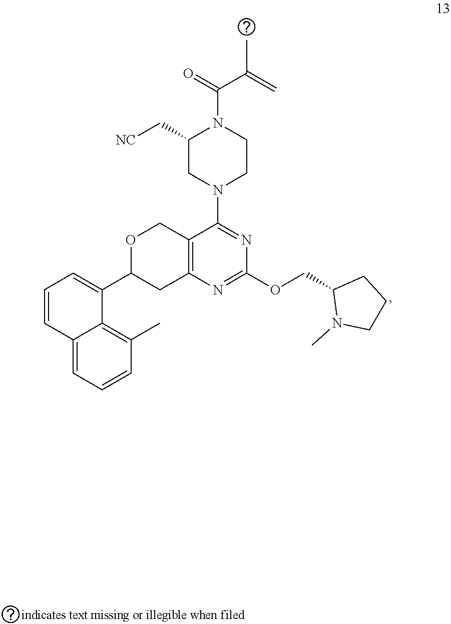 [SMILES: C=C(C)C(=O)N1CCN(c2nc(OC[C@@H]3CCCN3C)nc3c2COC(c2cccc4cccc(C)c24)C3)C[C@@H]1CC#N]

which has a retention time of 2.76 min under the following conditions: instrument: SFC Method Station (Thar. Waters): chromatographic column: OJ-H 4.6*100 mm, 5 μm (Daicel); column temperature: 40° C.; mobile phase; CO₂/MeOH (0.1% TEA)=60/40: flow rate: 4.0 ml/min: wavelength: 254 nm: back pressure; 120 bar;

compound

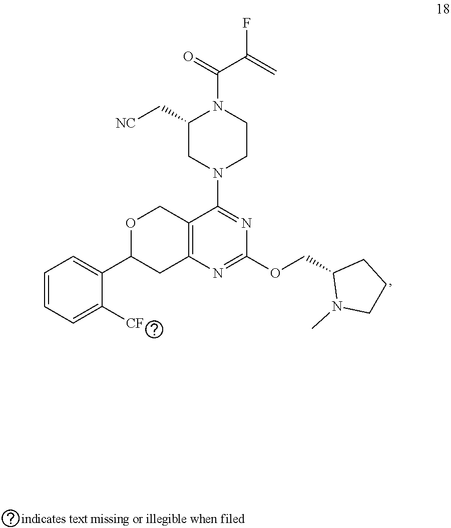

which has a retention time of 0.78 min under the following conditions: instrument: SFC Method Station (Thar. Waters): chromatographic OD-H 4.6*100 mm, 5 μm (Daicel): column temperature: 40° C.: mobile phase; CO₂/MeOH (0.1% TEA)=65/35: flow rate: 4.0 ml/min: wavelength: 254 nm: back pressure; 120 bar;

compound

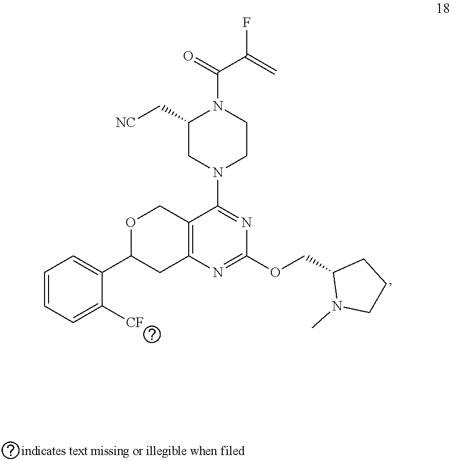

which has a retention time of 2.42 min under the following conditions: instrument: SFC Method Station (Thar. Waters): chromatographic column: OD-H 4.6*100 mm, 5 μm (Daicel): column temperature: 40° C.: mobile phase; CO₂/MeOH (0.1% TEA)=65/35: flow rate: 4.0 wavelength: 254 nm: back pressure; 120 bar;

compound

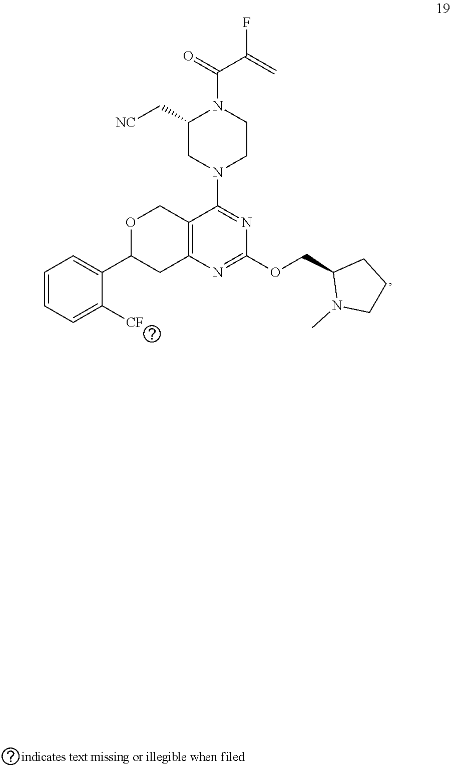

which has a retention time of 0.79 min under the following conditions: instrument: SFC Method Station (Thar. Waters): chromatographic column: OD-H 4.6*100 mm, 5 μm (Daicel): column temperature: 40° C.: mobile phase; CO₂/MeOH (0.1% TEA)=65/35: flow rate: 4.0 ml/min: wavelength: 254 inn: back pressure; 120 bar;

compound

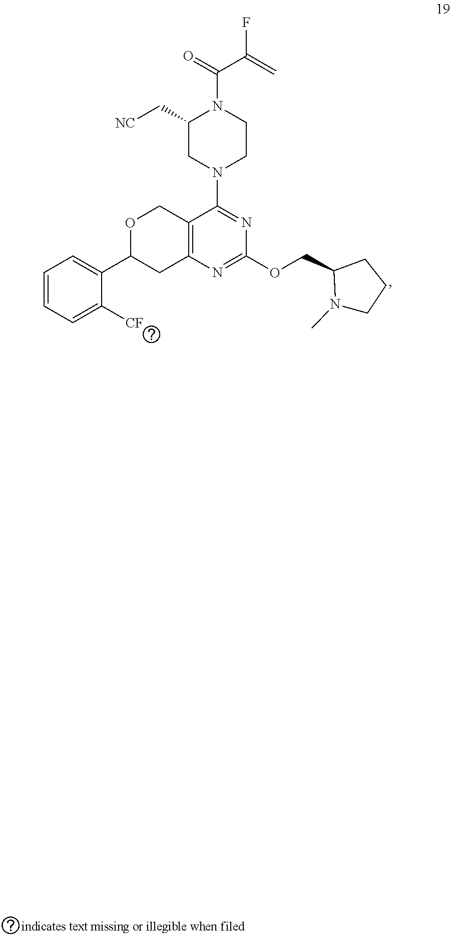

which has a retention time of 2.29 min under the following conditions: instrument: SFC Method Station (Thar. Waters): chromatographic column: OD-H 4.6*100 min, 5 μm (Daicel): column temperature: 40° C.: mobile phase; CO₂/MeOH (0.1% TEA)=65/35; flow rate: 4.0 ml/min: wavelength: 254 nm: back pressure; 120 bar;

compound

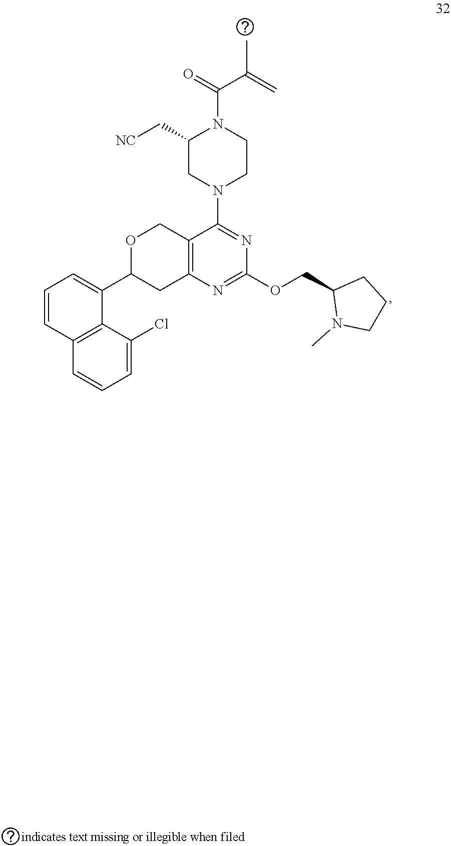 [SMILES: C=C(C)C(=O)N1CCN(c2nc(OC[C@H]3CCCN3C)nc3c2COC(c2cccc4cccc(Cl)c24)C3)C[C@@H]1CC#N]

which has a retention time of 1.45 min under the following conditions: instrument: SFC Method Station (Thar. Waters): chromatographic column: OJ-H 4.6*100 min, 5 μm (Daicel): column temperature: 40° C.: mobile phase; CO₂/MeOH (0.1% TEA)=60/40: flow rate: 4.0 ml/min: wavelength: 254 nm: back pressure; 120 bar;

compound

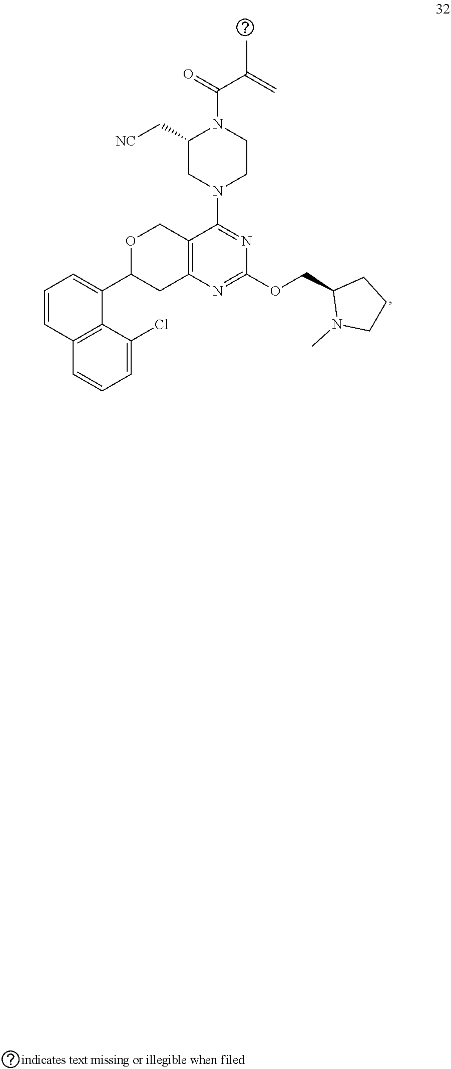 [SMILES: C=C(C)C(=O)N1CCN(c2nc(OC[C@H]3CCCN3C)nc3c2COC(c2cccc4cccc(Cl)c24)C3)C[C@@H]1CC#N]

which has a retention time of 2.81 min under the following conditions: instrument: SFC Method Station (Thar. Waters): chromatographic column: OJ-H 4.6*100 mm, 5 μm (Daicel): column temperature: 40° C.: mobile phase; CO₂/MeOH (0.1% TEA)=60/40: flow rate: 4.0 ml/min: wavelength: 254 nm: back pressure; 120 bar.

In a certain embodiment, the oxygen-containing heterocyclic compound represented by formula I is any one of the following compounds:

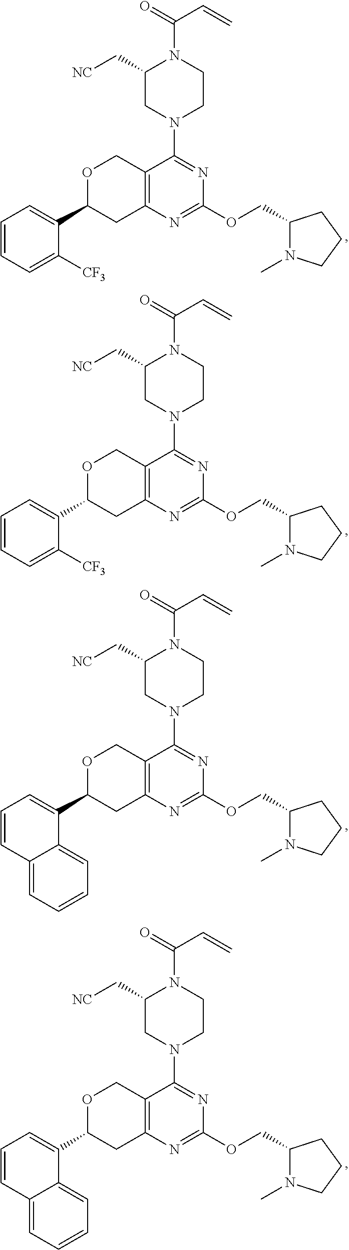 [SMILES: C=CC(=O)N1CCN(c2nc(OC[C@@H]3CCCN3C)nc3c2CO[C@H](c2ccccc2C(F)(F)F)C3)C[C@@H]1CC#N ; C=CC(=O)N1CCN(c2nc(OC[C@@H]3CCCN3C)nc3c2CO[C@@H](c2ccccc2C(F)(F)F)C3)C[C@@H]1CC#N ; C=CC(=O)N1CCN(c2nc(OC[C@@H]3CCCN3C)nc3c2CO[C@H](c2cccc4ccccc24)C3)C[C@@H]1CC#N ; C=CC(=O)N1CCN(c2nc(OC[C@@H]3CCCN3C)nc3c2CO[C@@H](c2cccc4ccccc24)C3)C[C@@H]1CC#N]

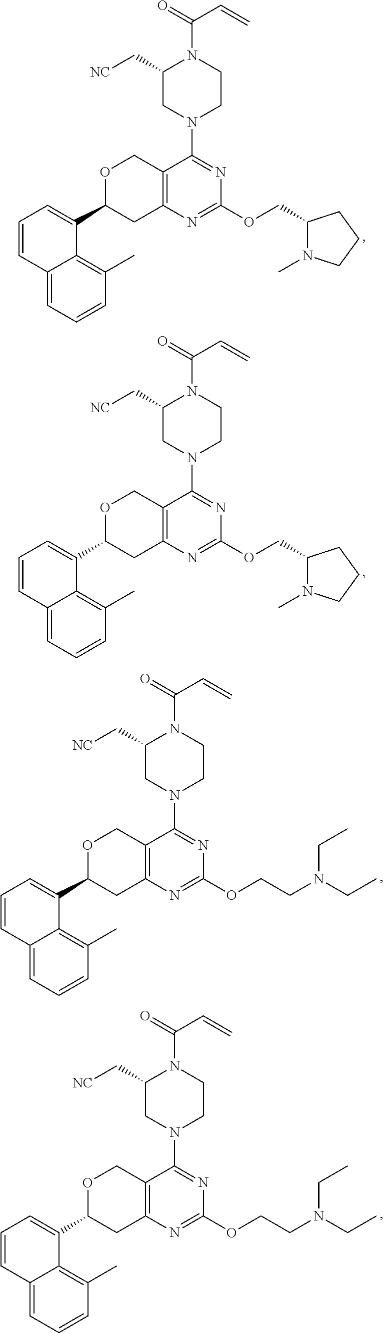 [SMILES: C=CC(=O)N1CCN(c2nc(OC[C@@H]3CCCN3C)nc3c2CO[C@H](c2cccc4cccc(C)c24)C3)C[C@@H]1CC#N ; C=CC(=O)N1CCN(c2nc(OC[C@@H]3CCCN3C)nc3c2CO[C@@H](c2cccc4cccc(C)c24)C3)C[C@@H]1CC#N ; C=CC(=O)N1CCN(c2nc(OCCN(CC)CC)nc3c2CO[C@H](c2cccc4cccc(C)c24)C3)C[C@@H]1CC#N ; C=CC(=O)N1CCN(c2nc(OCCN(CC)CC)nc3c2CO[C@@H](c2cccc4cccc(C)c24)C3)C[C@@H]1CC#N]

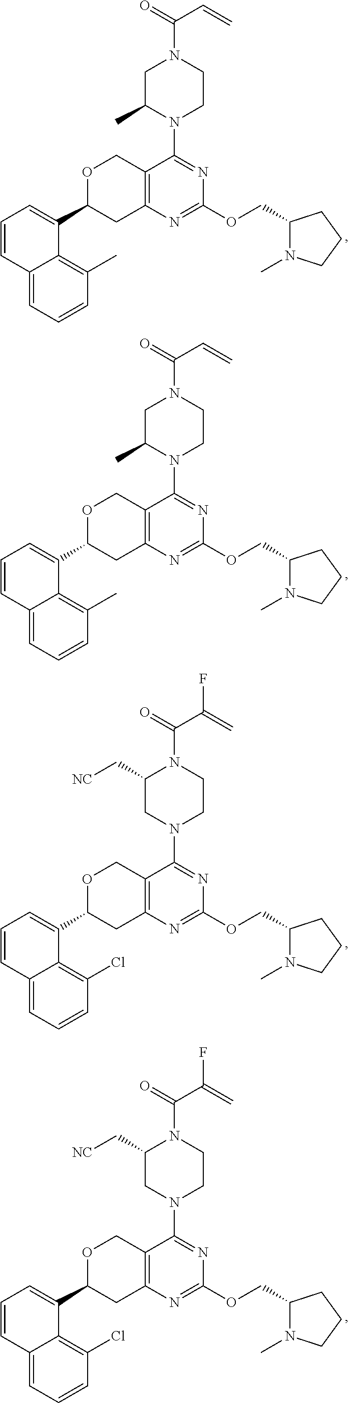 [SMILES: C=CC(=O)N1CCN(c2nc(OC[C@@H]3CCCN3C)nc3c2CO[C@H](c2cccc4cccc(C)c24)C3)[C@@H](C)C1 ; C=CC(=O)N1CCN(c2nc(OC[C@@H]3CCCN3C)nc3c2CO[C@@H](c2cccc4cccc(C)c24)C3)[C@@H](C)C1 ; C=C(F)C(=O)N1CCN(c2nc(OC[C@@H]3CCCN3C)nc3c2CO[C@@H](c2cccc4cccc(Cl)c24)C3)C[C@@H]1CC#N ; C=C(F)C(=O)N1CCN(c2nc(OC[C@@H]3CCCN3C)nc3c2CO[C@H](c2cccc4cccc(Cl)c24)C3)C[C@@H]1CC#N]

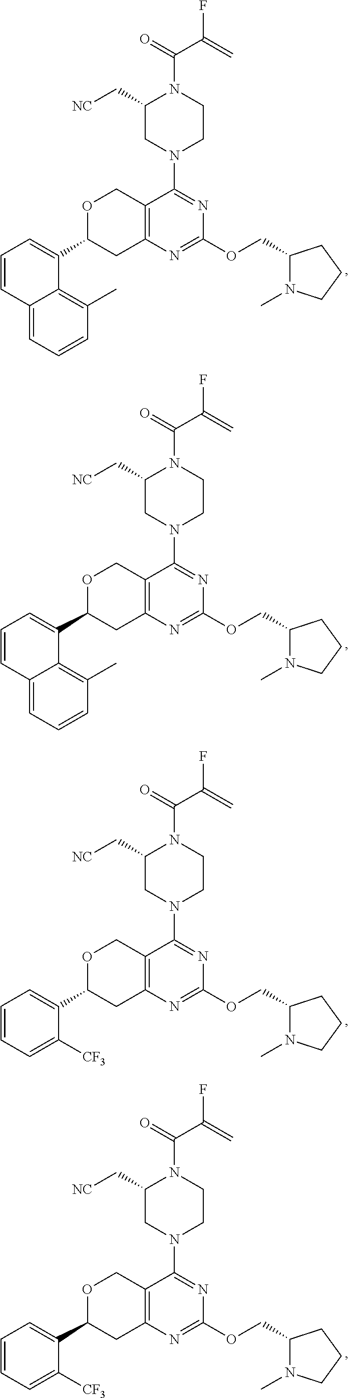 [SMILES: C=C(F)C(=O)N1CCN(c2nc(OC[C@@H]3CCCN3C)nc3c2CO[C@@H](c2cccc4cccc(C)c24)C3)C[C@@H]1CC#N ; C=C(F)C(=O)N1CCN(c2nc(OC[C@@H]3CCCN3C)nc3c2CO[C@H](c2cccc4cccc(C)c24)C3)C[C@@H]1CC#N ; C=C(F)C(=O)N1CCN(c2nc(OC[C@@H]3CCCN3C)nc3c2CO[C@@H](c2ccccc2C(F)(F)F)C3)C[C@@H]1CC#N ; C=C(F)C(=O)N1CCN(c2nc(OC[C@@H]3CCCN3C)nc3c2CO[C@H](c2ccccc2C(F)(F)F)C3)C[C@@H]1CC#N]

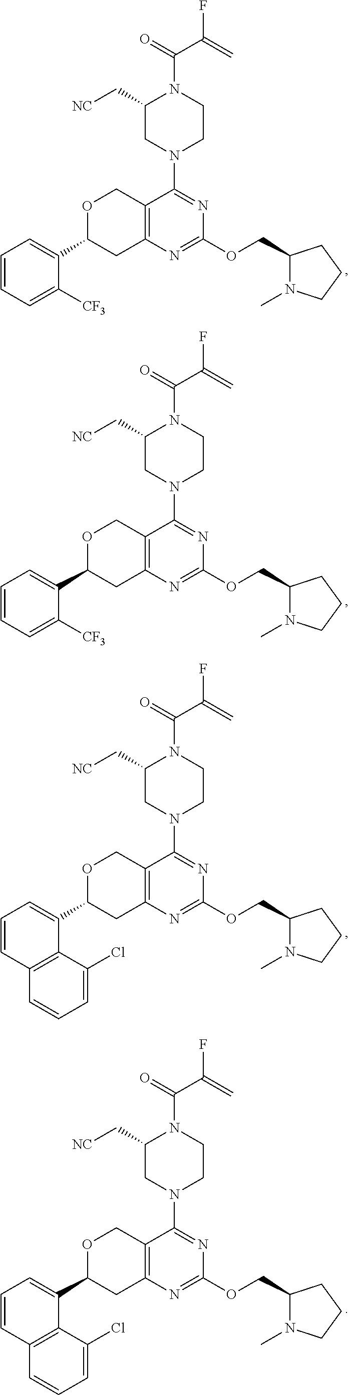 [SMILES: C=C(F)C(=O)N1CCN(c2nc(OC[C@H]3CCCN3C)nc3c2CO[C@@H](c2ccccc2C(F)(F)F)C3)C[C@@H]1CC#N ; C=C(F)C(=O)N1CCN(c2nc(OC[C@H]3CCCN3C)nc3c2CO[C@H](c2ccccc2C(F)(F)F)C3)C[C@@H]1CC#N ; C=C(F)C(=O)N1CCN(c2nc(OC[C@H]3CCCN3C)nc3c2CO[C@@H](c2cccc4cccc(Cl)c24)C3)C[C@@H]1CC#N ; C=C(F)C(=O)N1CCN(c2nc(OC[C@H]3CCCN3C)nc3c2CO[C@H](c2cccc4cccc(Cl)c24)C3)C[C@@H]1CC#N]

The present disclosure also provides a method for preparing the above-mentioned oxygen-containing heterocyclic compound represented by formula I, the method comprising route one or route two:

route one:

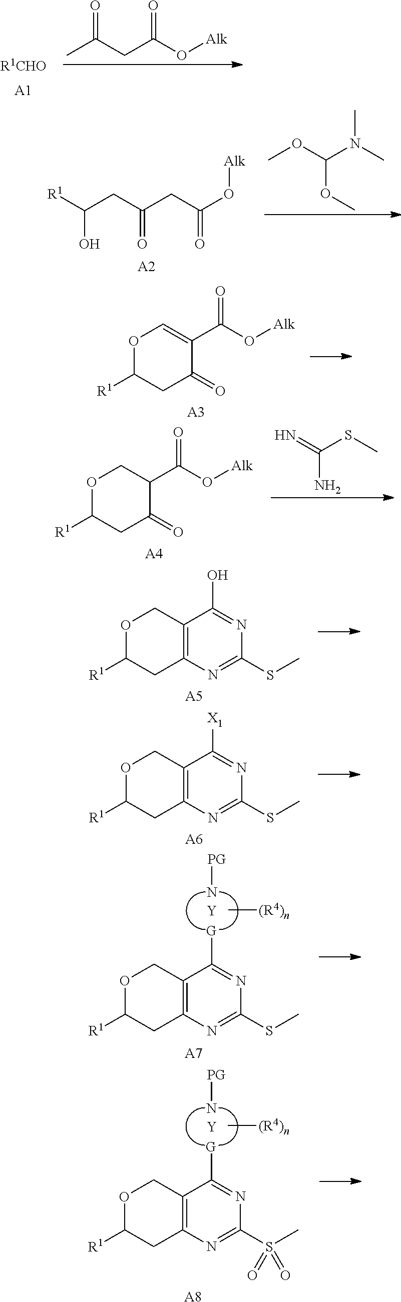

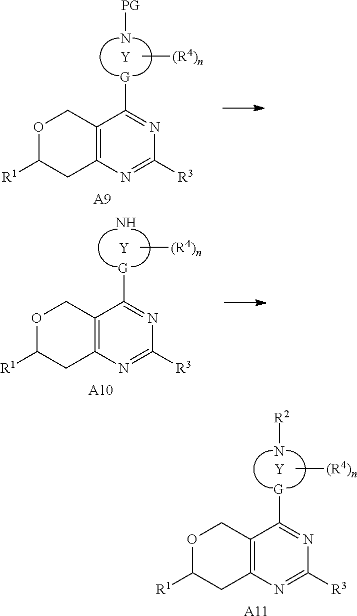

R¹, R², R³, R⁴, G, In and Y are all as defined above: X₁ is a leaving group (for example, OTf or Cl), etc.: Alk is alkyl (for example, C₁ alkyl): PG is an amino protecting group (for example, Boc or Cbz);

route two:

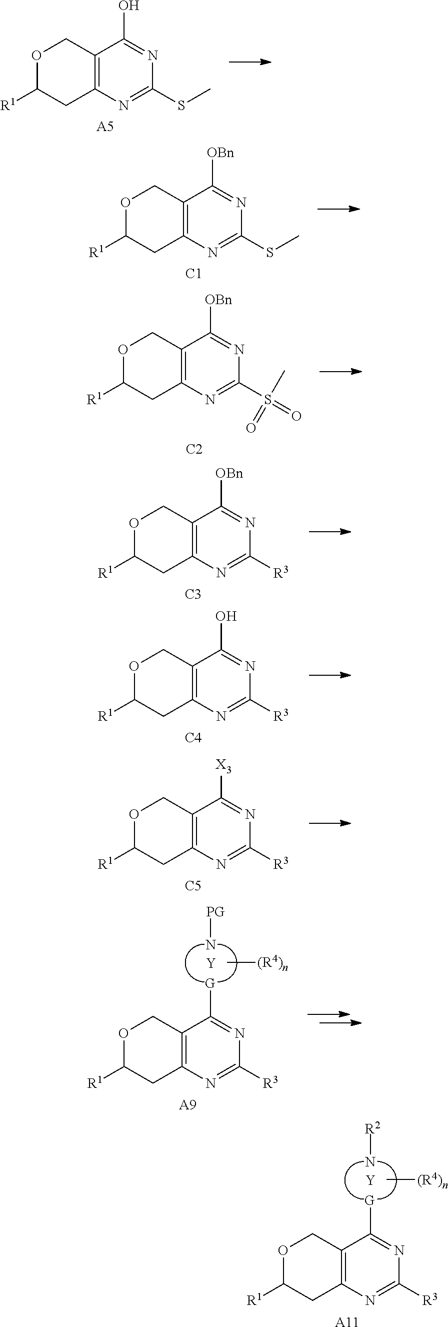

R¹, R², R³, R⁴, G, n and Y are all as defined above: X₃ is a leaving group (for example, OTf or Cl): PG is an amino protecting group (for example, Boc or Cbz).

The route one is described in detail as follows: aldehyde compound A1 is condensed with acetyl acetate to obtain compound A2: A2 is condensed with DMF-DMA to obtain compound A3: A3 is reduced to A4: A5 is obtained from A4 after ring formation: the hydroxyl group in A5 is converted into a leaving group and A6 is obtained; A6 is converted into A7 by means of nucleophilic substitution, coupling, etc.: A7 is oxidized to obtain A8: A8 is further converted into A9: and A9 is deprotected and further converted into A11.

The route two is described in detail as follows: compound A5 is protected by Ba: C1 is oxidized to obtain C2; C2 is converted into C3 by nucleophilic substitution; C3 is subjected to Bn deprotection and converted into C4: the hydroxyl group in C4 is converted into a leaving group and C5 is obtained: C5 is converted into A9 by means of nucleophilic substitution, coupling, etc.; A9 is deprotected and further converted into A11.

The conditions and steps adopted for the chemical reactions involved in the various reaction routes described in the present disclosure all can be carried out with reference to conventional conditions and steps for such reactions in the art, and specific reference may be made to the literatures: R. Larock. Comprehensive Organic Transformations, VCH Publishers (1989); T. W. Greene and P. G. M. Wuts, Protective Groups in Organic Synthesis, 3^{rd} ED., John Wiley and Sons (1999): L. Fieser and M. Fieser, Fieser and Fieser's Reagents for Organic Synthesis, John Wiley and Sons (1994); L. Paquette, ed., Encyclopedia of Reagents for Organic Synthesis. John Wiley and Sons (1995) subsequent versions.

The present application cites the entire content of the above literatures. In addition, other target compounds of the present disclosure can also be obtained from the compounds obtained by the above-mentioned methods through modifying peripheral positions with reference to related methods of the above literatures.

The present disclosure also provides a compound represented by formula A5, A6, A7, A8, A9, A10, C1, C2, C3, C4 or C5;

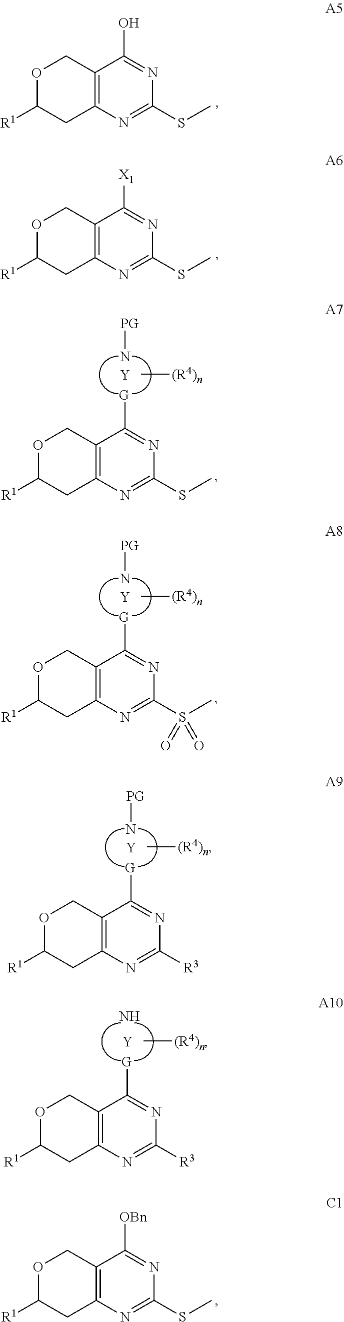

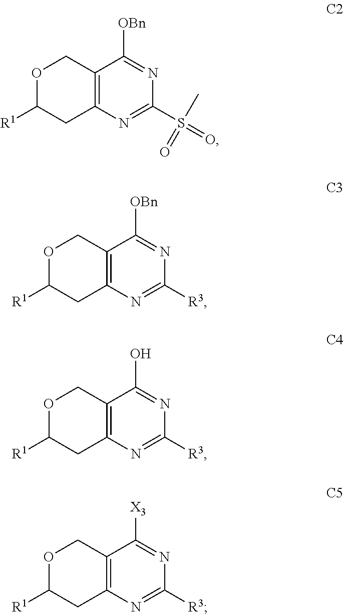

wherein R¹, R³, R⁴, G, Y and n are as defined above:

X¹ and X³ are independently leaving groups (for example, OTf or Cl): PG is an amino protecting group (for example, Boc or Cbz).

In a certain embodiment, the compound represented by formula A5, A6, A7, A8, A9, A10, C1, C2, C3, C4 or C5 may be any one of the following compounds:

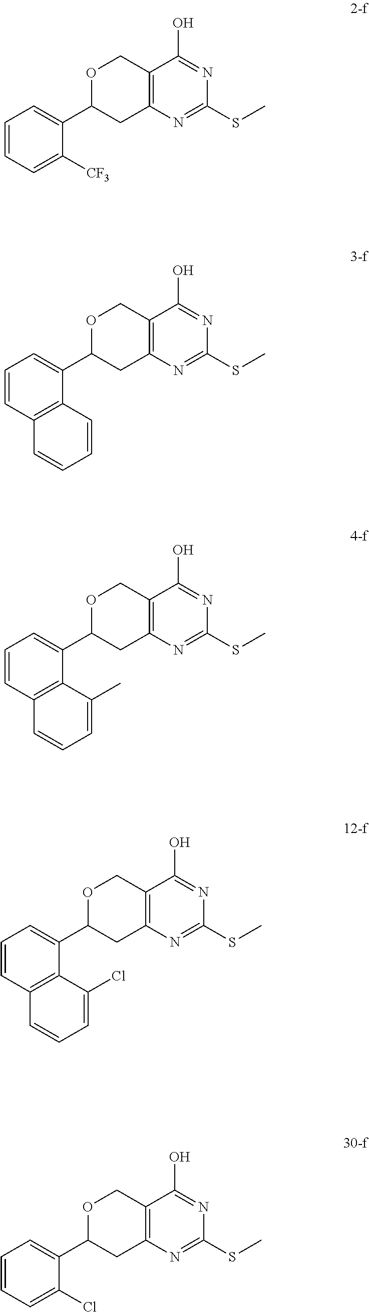 [SMILES: CSc1nc(O)c2c(n1)CC(c1ccccc1C(F)(F)F)OC2 ; CSc1nc(O)c2c(n1)CC(c1cccc3ccccc13)OC2 ; CSc1nc(O)c2c(n1)CC(c1cccc3cccc(C)c13)OC2 ; CSc1nc(O)c2c(n1)CC(c1cccc3cccc(Cl)c13)OC2 ; CSc1nc(O)c2c(n1)CC(c1ccccc1Cl)OC2]

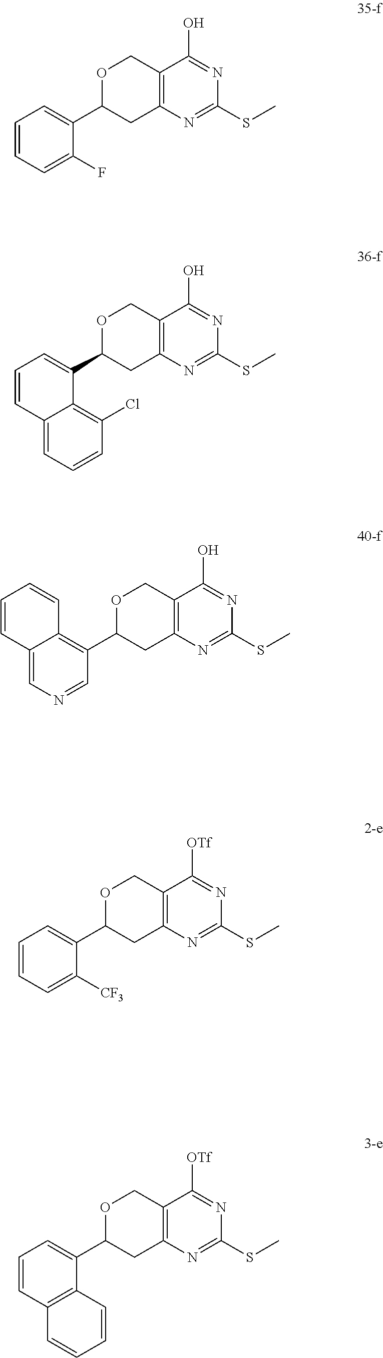 [SMILES: CSc1nc(O)c2c(n1)CC(c1ccccc1F)OC2 ; CSc1nc(O)c2c(n1)C[C@@H](c1cccc3cccc(Cl)c13)OC2 ; CSc1nc(O)c2c(n1)CC(c1cncc3ccccc13)OC2 ; CSc1nc2c(c(OS(=O)(=O)C(F)(F)F)n1)COC(c1ccccc1C(F)(F)F)C2 ; CSc1nc2c(c(OS(=O)(=O)C(F)(F)F)n1)COC(c1cccc3ccccc13)C2]

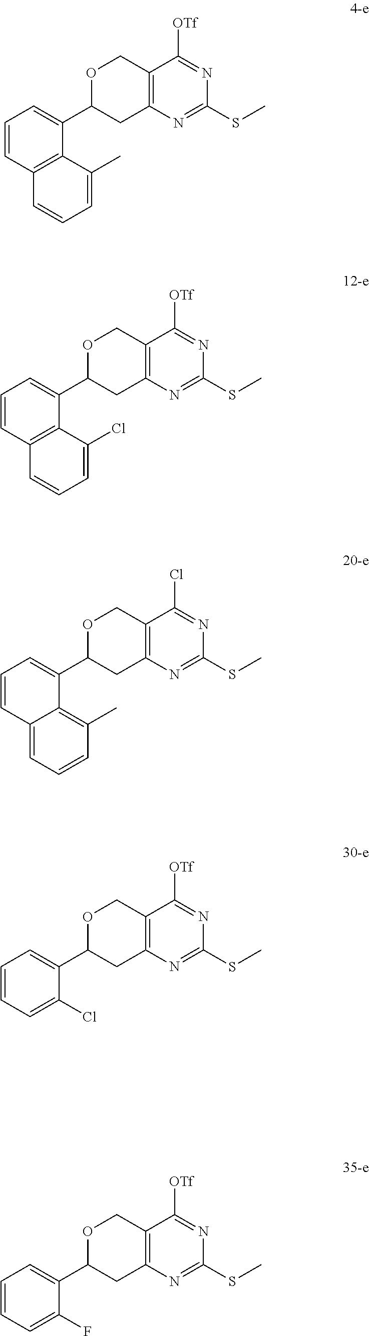 [SMILES: CSc1nc2c(c(OS(=O)(=O)C(F)(F)F)n1)COC(c1cccc3cccc(C)c13)C2 ; CSc1nc2c(c(OS(=O)(=O)C(F)(F)F)n1)COC(c1cccc3cccc(Cl)c13)C2 ; CSc1nc(Cl)c2c(n1)CC(c1cccc3cccc(C)c13)OC2 ; CSc1nc2c(c(OS(=O)(=O)C(F)(F)F)n1)COC(c1ccccc1Cl)C2 ; CSc1nc2c(c(OS(=O)(=O)C(F)(F)F)n1)COC(c1ccccc1F)C2]

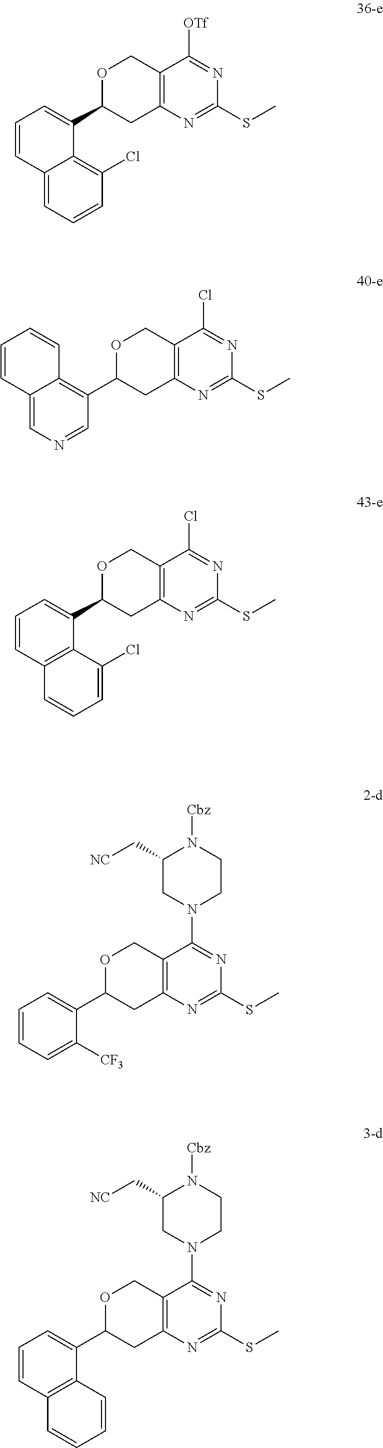 [SMILES: CSc1nc2c(c(OS(=O)(=O)C(F)(F)F)n1)CO[C@H](c1cccc3cccc(Cl)c13)C2 ; CSc1nc(Cl)c2c(n1)CC(c1cncc3ccccc13)OC2 ; CSc1nc(Cl)c2c(n1)C[C@@H](c1cccc3cccc(Cl)c13)OC2 ; CSc1nc2c(c(N3CCN(C(=O)OCc4ccccc4)[C@@H](CC#N)C3)n1)COC(c1ccccc1C(F)(F)F)C2 ; CSc1nc2c(c(N3CCN(C(=O)OCc4ccccc4)[C@@H](CC#N)C3)n1)COC(c1cccc3ccccc13)C2]

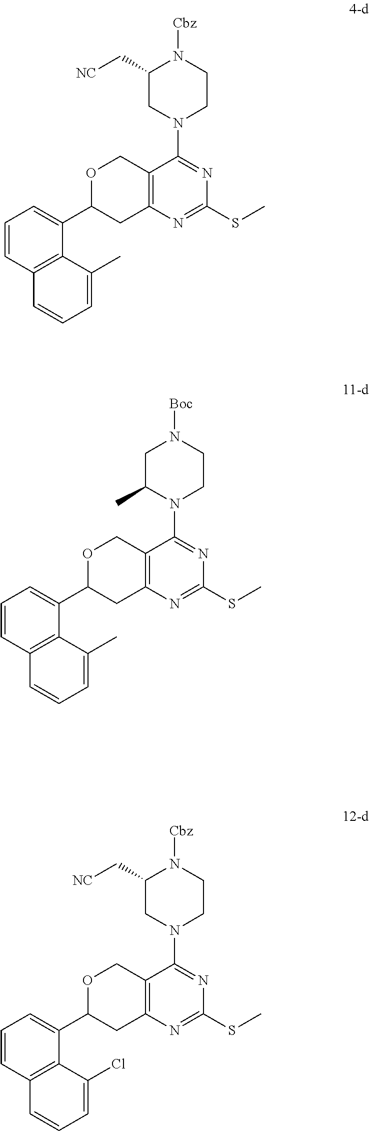 [SMILES: CSc1nc2c(c(N3CCN(C(=O)OCc4ccccc4)[C@@H](CC#N)C3)n1)COC(c1cccc3cccc(C)c13)C2 ; CSc1nc2c(c(N3CCN(C(=O)OC(C)(C)C)C[C@@H]3C)n1)COC(c1cccc3cccc(C)c13)C2 ; CSc1nc2c(c(N3CCN(C(=O)OCc4ccccc4)[C@@H](CC#N)C3)n1)COC(c1cccc3cccc(Cl)c13)C2]

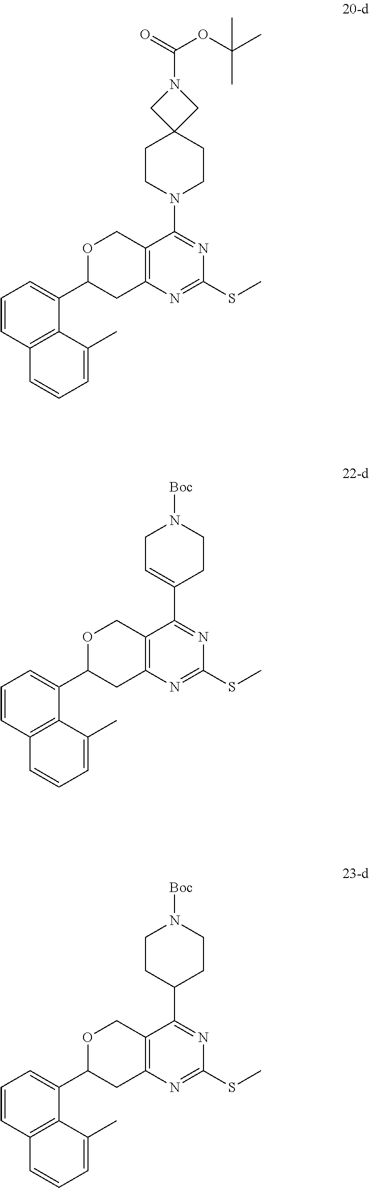 [SMILES: CSc1nc2c(c(N3CCC4(CC3)CN(C(=O)OC(C)(C)C)C4)n1)COC(c1cccc3cccc(C)c13)C2 ; CSc1nc2c(c(C3=CCN(C(=O)OC(C)(C)C)CC3)n1)COC(c1cccc3cccc(C)c13)C2 ; CSc1nc2c(c(C3CCN(C(=O)OC(C)(C)C)CC3)n1)COC(c1cccc3cccc(C)c13)C2]

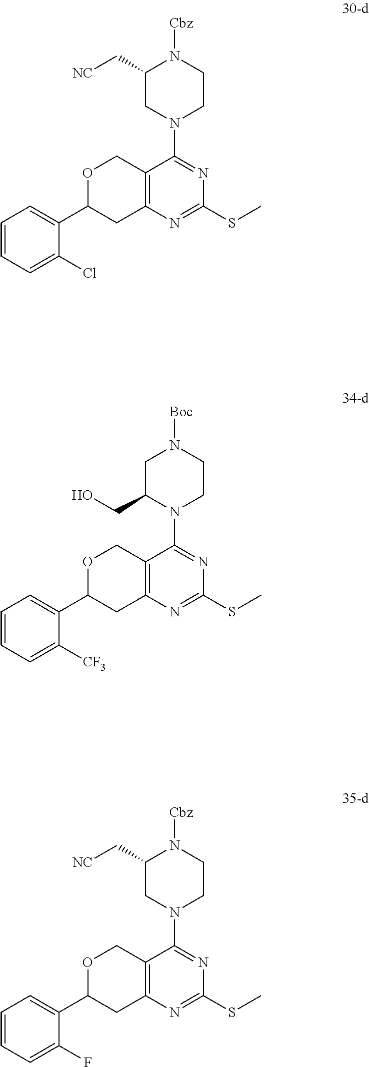 [SMILES: CSc1nc2c(c(N3CCN(C(=O)OCc4ccccc4)[C@@H](CC#N)C3)n1)COC(c1ccccc1Cl)C2 ; CSc1nc2c(c(N3CCN(C(=O)OC(C)(C)C)C[C@@H]3CO)n1)COC(c1ccccc1C(F)(F)F)C2 ; CSc1nc2c(c(N3CCN(C(=O)OCc4ccccc4)[C@@H](CC#N)C3)n1)COC(c1ccccc1F)C2]

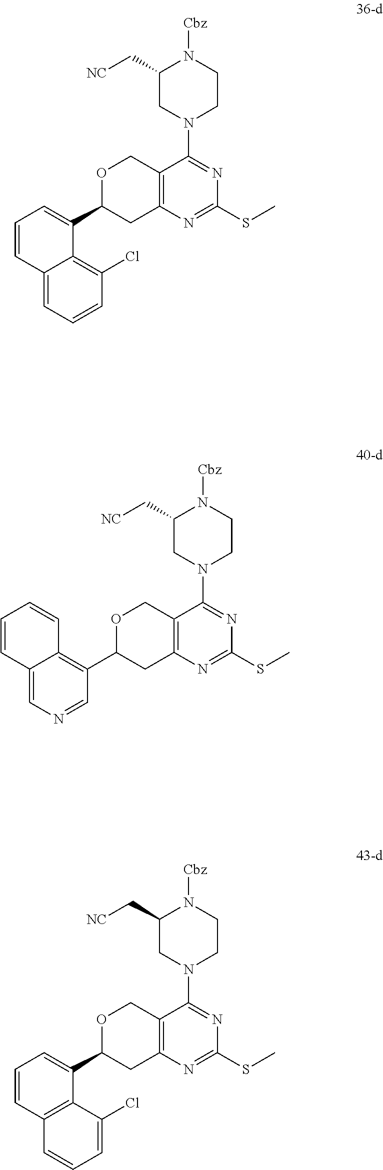 [SMILES: CSc1nc2c(c(N3CCN(C(=O)OCc4ccccc4)[C@@H](CC#N)C3)n1)CO[C@H](c1cccc3cccc(Cl)c13)C2 ; CSc1nc2c(c(N3CCN(C(=O)OCc4ccccc4)[C@@H](CC#N)C3)n1)COC(c1cncc3ccccc13)C2 ; CSc1nc2c(c(N3CCN(C(=O)OCc4ccccc4)[C@H](CC#N)C3)n1)CO[C@H](c1cccc3cccc(Cl)c13)C2]

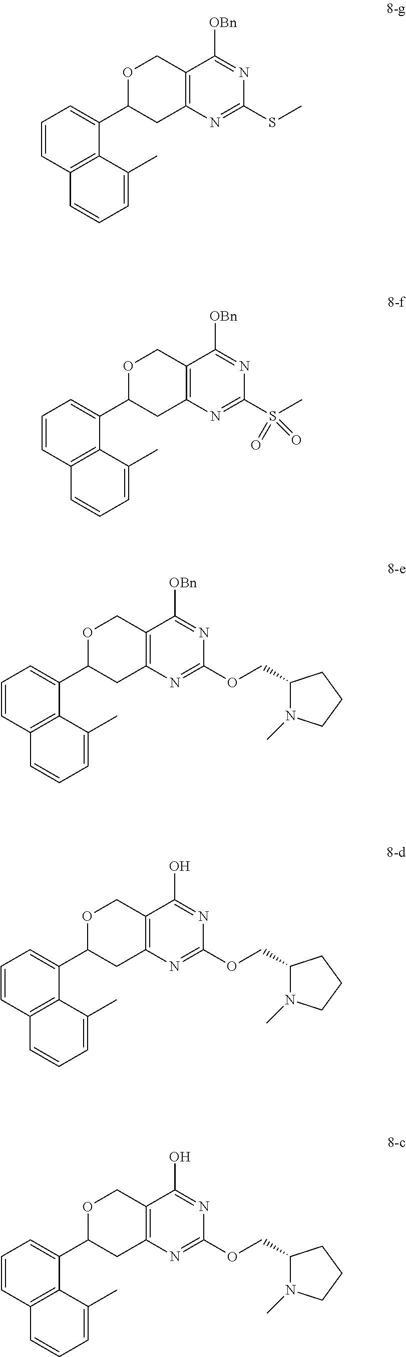 [SMILES: CSc1nc2c(c(OCc3ccccc3)n1)COC(c1cccc3cccc(C)c13)C2 ; Cc1cccc2cccc(C3Cc4nc(S(C)(=O)=O)nc(OCc5ccccc5)c4CO3)c12 ; Cc1cccc2cccc(C3Cc4nc(OC[C@@H]5CCCN5C)nc(OCc5ccccc5)c4CO3)c12 ; Cc1cccc2cccc(C3Cc4nc(OC[C@@H]5CCCN5C)nc(O)c4CO3)c12 ; Cc1cccc2cccc(C3Cc4nc(OC[C@@H]5CCCN5C)nc(O)c4CO3)c12]

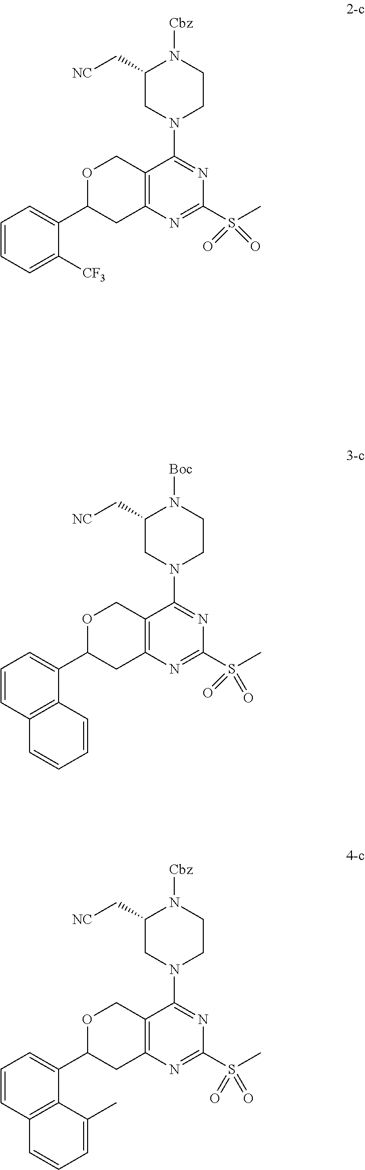 [SMILES: CS(=O)(=O)c1nc2c(c(N3CCN(C(=O)OCc4ccccc4)[C@@H](CC#N)C3)n1)COC(c1ccccc1C(F)(F)F)C2 ; CC(C)(C)OC(=O)N1CCN(c2nc(S(C)(=O)=O)nc3c2COC(c2cccc4ccccc24)C3)C[C@@H]1CC#N ; Cc1cccc2cccc(C3Cc4nc(S(C)(=O)=O)nc(N5CCN(C(=O)OCc6ccccc6)[C@@H](CC#N)C5)c4CO3)c12]

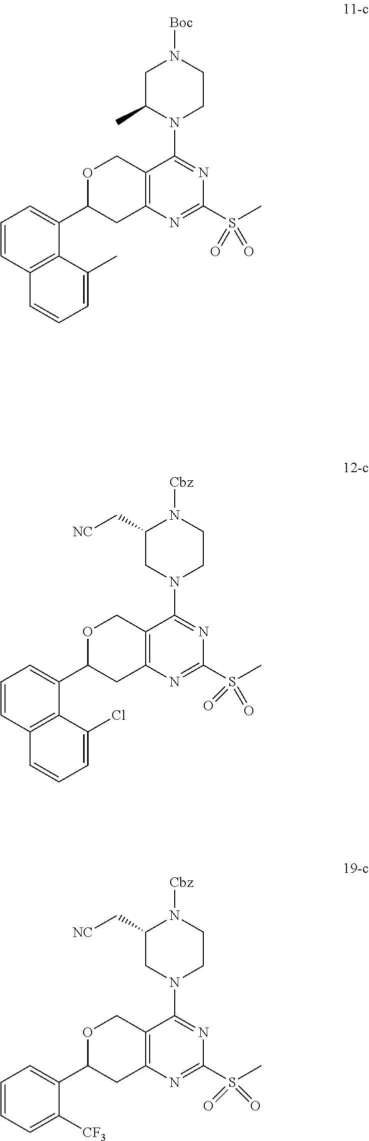 [SMILES: Cc1cccc2cccc(C3Cc4nc(S(C)(=O)=O)nc(N5CCN(C(=O)OC(C)(C)C)C[C@@H]5C)c4CO3)c12 ; CS(=O)(=O)c1nc2c(c(N3CCN(C(=O)OCc4ccccc4)[C@@H](CC#N)C3)n1)COC(c1cccc3cccc(Cl)c13)C2 ; CS(=O)(=O)c1nc2c(c(N3CCN(C(=O)OCc4ccccc4)[C@@H](CC#N)C3)n1)COC(c1ccccc1C(F)(F)F)C2]

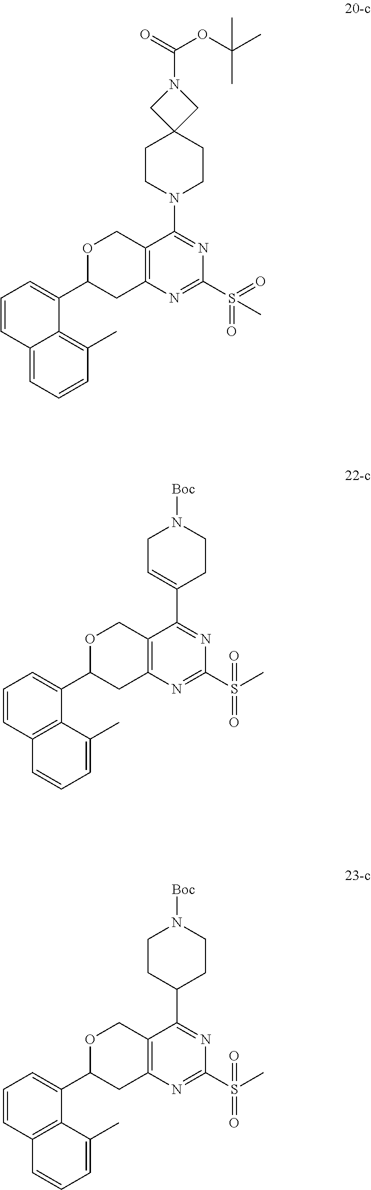 [SMILES: Cc1cccc2cccc(C3Cc4nc(S(C)(=O)=O)nc(N5CCC6(CC5)CN(C(=O)OC(C)(C)C)C6)c4CO3)c12 ; Cc1cccc2cccc(C3Cc4nc(S(C)(=O)=O)nc(C5=CCN(C(=O)OC(C)(C)C)CC5)c4CO3)c12 ; Cc1cccc2cccc(C3Cc4nc(S(C)(=O)=O)nc(C5CCN(C(=O)OC(C)(C)C)CC5)c4CO3)c12]

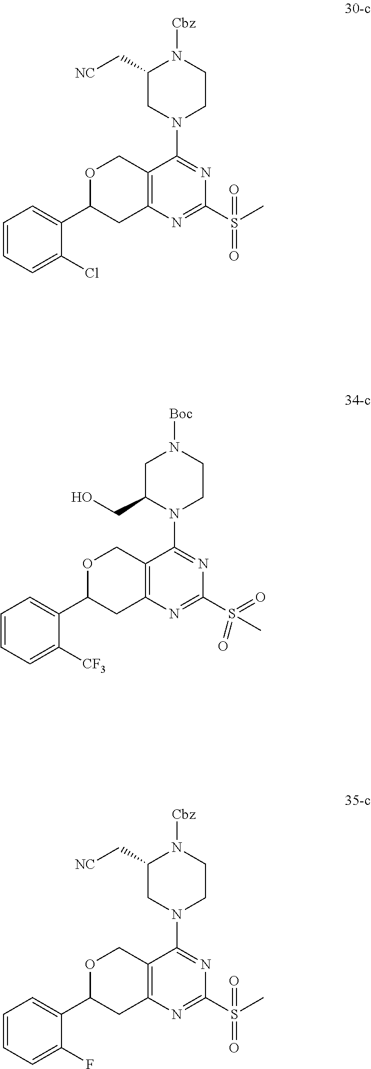 [SMILES: CS(=O)(=O)c1nc2c(c(N3CCN(C(=O)OCc4ccccc4)[C@@H](CC#N)C3)n1)COC(c1ccccc1Cl)C2 ; CC(C)(C)OC(=O)N1CCN(c2nc(S(C)(=O)=O)nc3c2COC(c2ccccc2C(F)(F)F)C3)[C@@H](CO)C1 ; CS(=O)(=O)c1nc2c(c(N3CCN(C(=O)OCc4ccccc4)[C@@H](CC#N)C3)n1)COC(c1ccccc1F)C2]

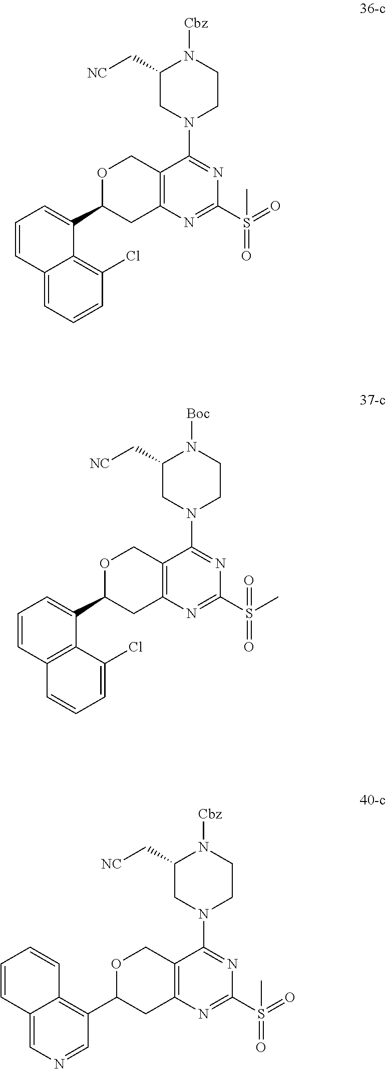 [SMILES: CS(=O)(=O)c1nc2c(c(N3CCN(C(=O)OCc4ccccc4)[C@@H](CC#N)C3)n1)CO[C@H](c1cccc3cccc(Cl)c13)C2 ; CC(C)(C)OC(=O)N1CCN(c2nc(S(C)(=O)=O)nc3c2CO[C@H](c2cccc4cccc(Cl)c24)C3)C[C@@H]1CC#N ; CS(=O)(=O)c1nc2c(c(N3CCN(C(=O)OCc4ccccc4)[C@@H](CC#N)C3)n1)COC(c1cncc3ccccc13)C2]

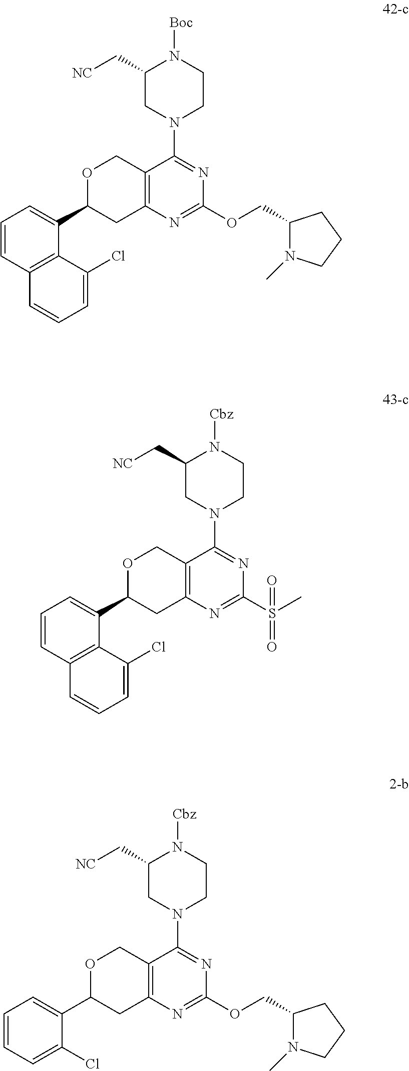 [SMILES: CN1CCC[C@H]1COc1nc2c(c(N3CCN(C(=O)OC(C)(C)C)[C@@H](CC#N)C3)n1)CO[C@H](c1cccc3cccc(Cl)c13)C2 ; CS(=O)(=O)c1nc2c(c(N3CCN(C(=O)OCc4ccccc4)[C@H](CC#N)C3)n1)CO[C@H](c1cccc3cccc(Cl)c13)C2 ; CN1CCC[C@H]1COc1nc2c(c(N3CCN(C(=O)OCc4ccccc4)[C@@H](CC#N)C3)n1)COC(c1ccccc1Cl)C2]

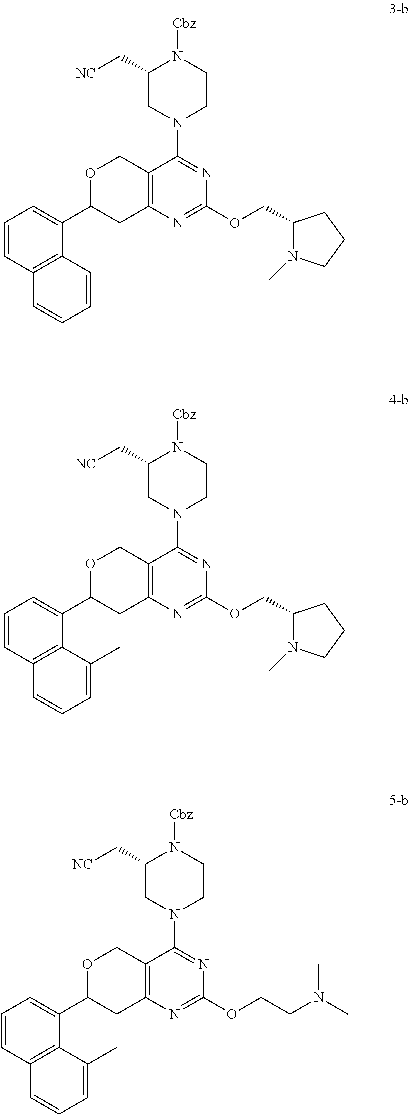 [SMILES: CN1CCC[C@H]1COc1nc2c(c(N3CCN(C(=O)OCc4ccccc4)[C@@H](CC#N)C3)n1)COC(c1cccc3ccccc13)C2 ; Cc1cccc2cccc(C3Cc4nc(OC[C@@H]5CCCN5C)nc(N5CCN(C(=O)OCc6ccccc6)[C@@H](CC#N)C5)c4CO3)c12 ; Cc1cccc2cccc(C3Cc4nc(OCCN(C)C)nc(N5CCN(C(=O)OCc6ccccc6)[C@@H](CC#N)C5)c4CO3)c12]

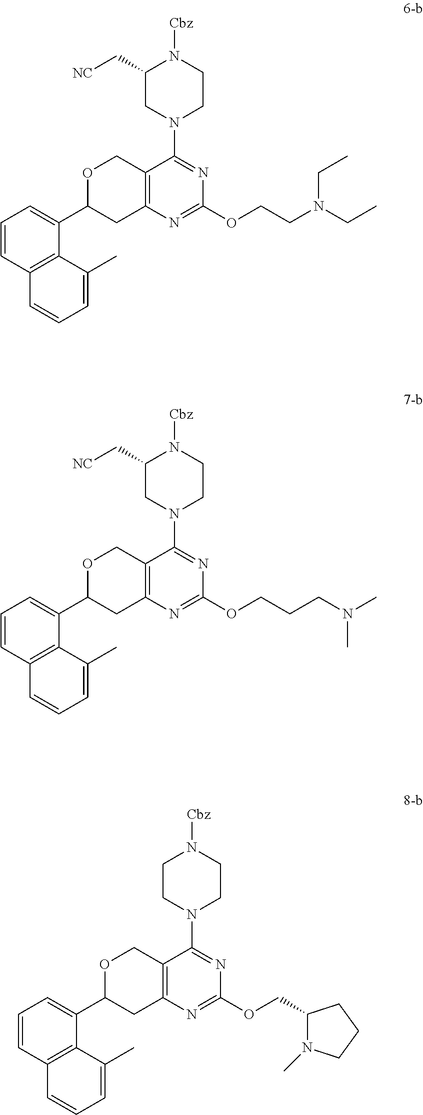 [SMILES: CCN(CC)CCOc1nc2c(c(N3CCN(C(=O)OCc4ccccc4)[C@@H](CC#N)C3)n1)COC(c1cccc3cccc(C)c13)C2 ; Cc1cccc2cccc(C3Cc4nc(OCCCN(C)C)nc(N5CCN(C(=O)OCc6ccccc6)[C@@H](CC#N)C5)c4CO3)c12 ; Cc1cccc2cccc(C3Cc4nc(OC[C@@H]5CCCN5C)nc(N5CCN(C(=O)OCc6ccccc6)CC5)c4CO3)c12]

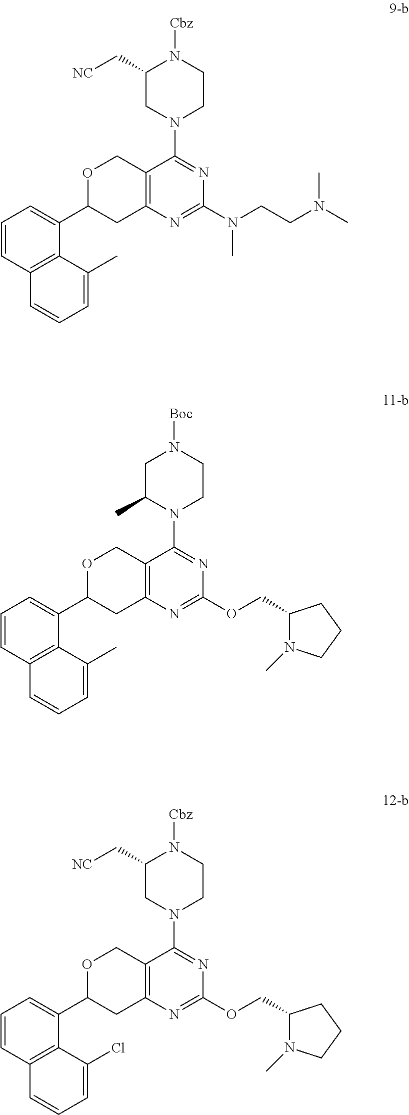 [SMILES: Cc1cccc2cccc(C3Cc4nc(N(C)CCN(C)C)nc(N5CCN(C(=O)OCc6ccccc6)[C@@H](CC#N)C5)c4CO3)c12 ; Cc1cccc2cccc(C3Cc4nc(OC[C@@H]5CCCN5C)nc(N5CCN(C(=O)OC(C)(C)C)C[C@@H]5C)c4CO3)c12 ; CN1CCC[C@H]1COc1nc2c(c(N3CCN(C(=O)OCc4ccccc4)[C@@H](CC#N)C3)n1)COC(c1cccc3cccc(Cl)c13)C2]

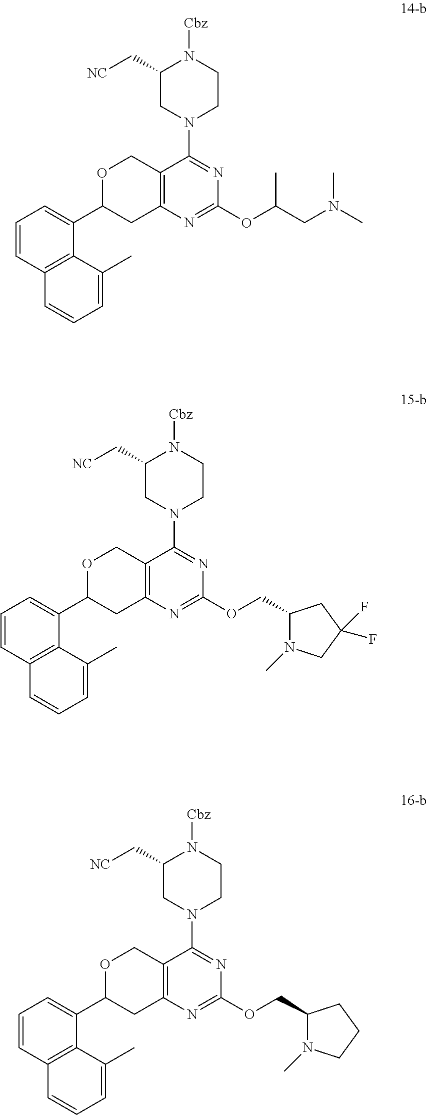 [SMILES: Cc1cccc2cccc(C3Cc4nc(OC(C)CN(C)C)nc(N5CCN(C(=O)OCc6ccccc6)[C@@H](CC#N)C5)c4CO3)c12 ; Cc1cccc2cccc(C3Cc4nc(OC[C@@H]5CC(F)(F)CN5C)nc(N5CCN(C(=O)OCc6ccccc6)[C@@H](CC#N)C5)c4CO3)c12 ; Cc1cccc2cccc(C3Cc4nc(OC[C@H]5CCCN5C)nc(N5CCN(C(=O)OCc6ccccc6)[C@@H](CC#N)C5)c4CO3)c12]

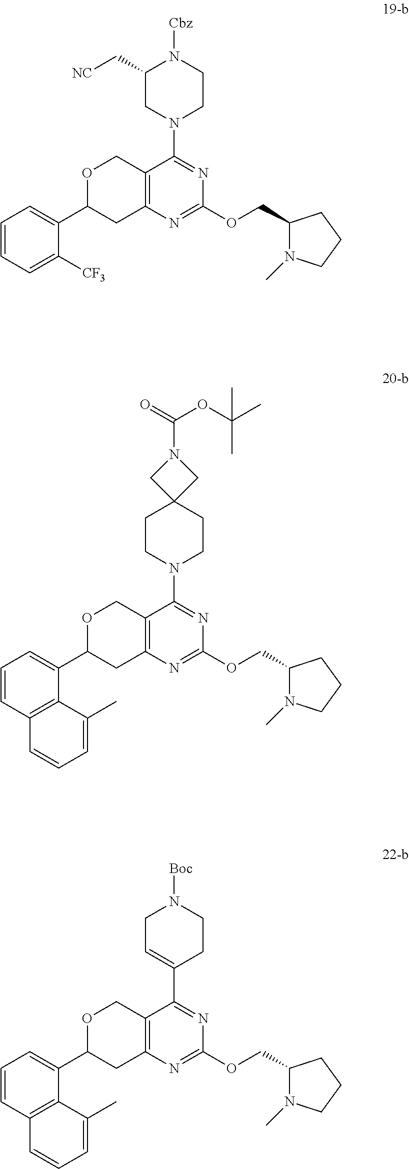 [SMILES: CN1CCC[C@@H]1COc1nc2c(c(N3CCN(C(=O)OCc4ccccc4)[C@@H](CC#N)C3)n1)COC(c1ccccc1C(F)(F)F)C2 ; Cc1cccc2cccc(C3Cc4nc(OC[C@@H]5CCCN5C)nc(N5CCC6(CC5)CN(C(=O)OC(C)(C)C)C6)c4CO3)c12 ; Cc1cccc2cccc(C3Cc4nc(OC[C@@H]5CCCN5C)nc(C5=CCN(C(=O)OC(C)(C)C)CC5)c4CO3)c12]

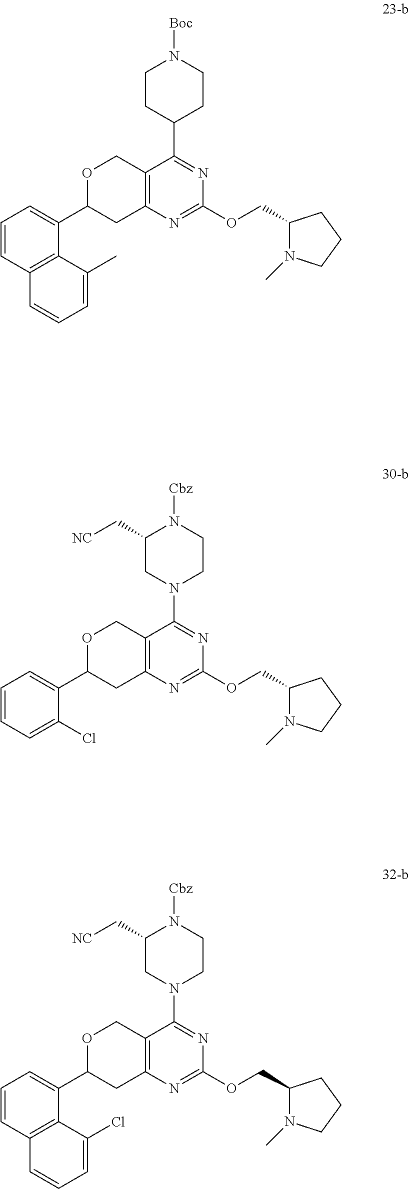 [SMILES: Cc1cccc2cccc(C3Cc4nc(OC[C@@H]5CCCN5C)nc(C5CCN(C(=O)OC(C)(C)C)CC5)c4CO3)c12 ; CN1CCC[C@H]1COc1nc2c(c(N3CCN(C(=O)OCc4ccccc4)[C@@H](CC#N)C3)n1)COC(c1ccccc1Cl)C2 ; CN1CCC[C@@H]1COc1nc2c(c(N3CCN(C(=O)OCc4ccccc4)[C@@H](CC#N)C3)n1)COC(c1cccc3cccc(Cl)c13)C2]

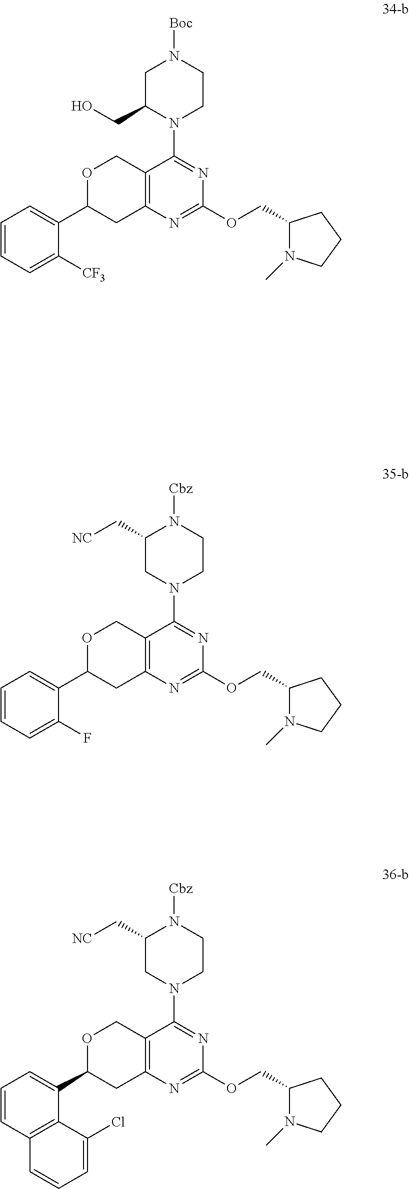 [SMILES: CN1CCC[C@H]1COc1nc2c(c(N3CCN(C(=O)OC(C)(C)C)C[C@@H]3CO)n1)COC(c1ccccc1C(F)(F)F)C2 ; CN1CCC[C@H]1COc1nc2c(c(N3CCN(C(=O)OCc4ccccc4)[C@@H](CC#N)C3)n1)COC(c1ccccc1F)C2 ; CN1CCC[C@H]1COc1nc2c(c(N3CCN(C(=O)OCc4ccccc4)[C@@H](CC#N)C3)n1)CO[C@H](c1cccc3cccc(Cl)c13)C2]

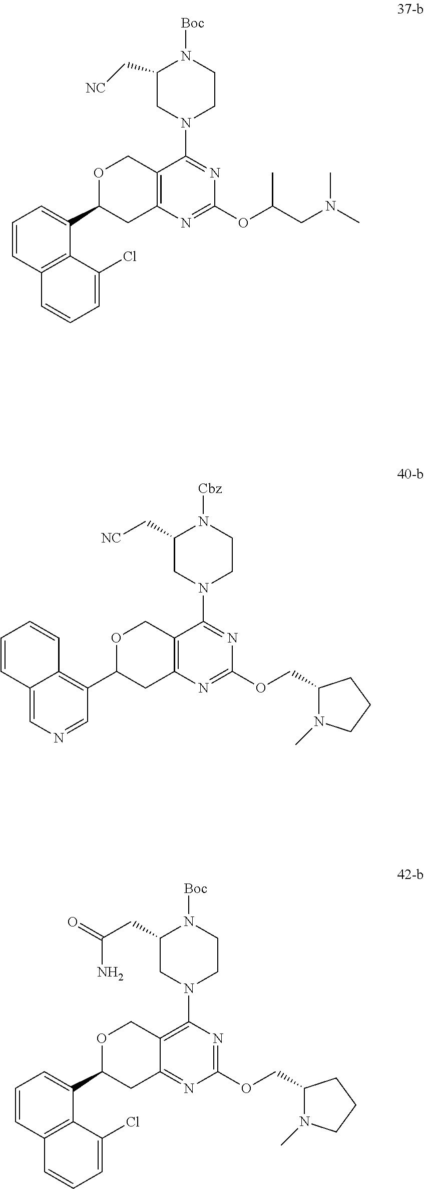 [SMILES: CC(CN(C)C)Oc1nc2c(c(N3CCN(C(=O)OC(C)(C)C)[C@@H](CC#N)C3)n1)CO[C@H](c1cccc3cccc(Cl)c13)C2 ; CN1CCC[C@H]1COc1nc2c(c(N3CCN(C(=O)OCc4ccccc4)[C@@H](CC#N)C3)n1)COC(c1cncc3ccccc13)C2 ; CN1CCC[C@H]1COc1nc2c(c(N3CCN(C(=O)OC(C)(C)C)[C@@H](CC(N)=O)C3)n1)CO[C@H](c1cccc3cccc(Cl)c13)C2]

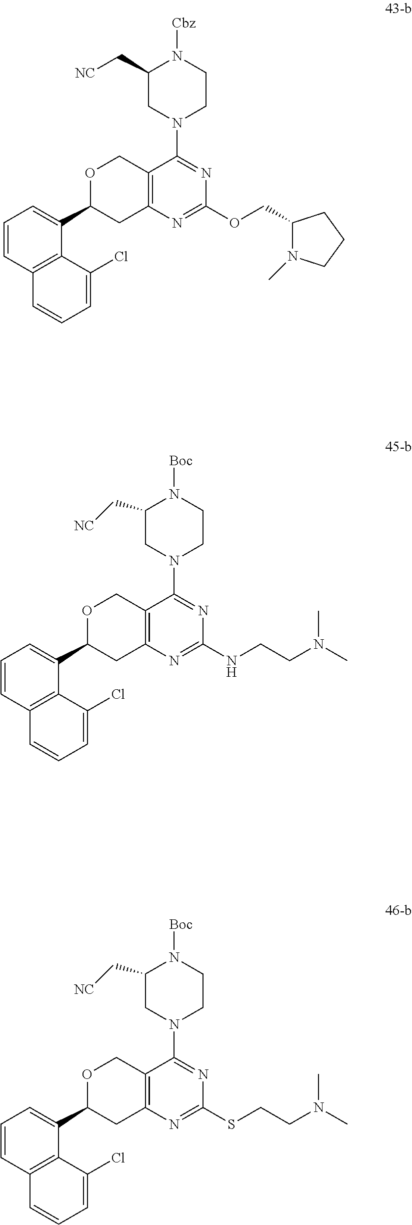 [SMILES: CN1CCC[C@H]1COc1nc2c(c(N3CCN(C(=O)OCc4ccccc4)[C@H](CC#N)C3)n1)CO[C@H](c1cccc3cccc(Cl)c13)C2 ; CN(C)CCNc1nc2c(c(N3CCN(C(=O)OC(C)(C)C)[C@@H](CC#N)C3)n1)CO[C@H](c1cccc3cccc(Cl)c13)C2 ; CN(C)CCSc1nc2c(c(N3CCN(C(=O)OC(C)(C)C)[C@@H](CC#N)C3)n1)CO[C@H](c1cccc3cccc(Cl)c13)C2]

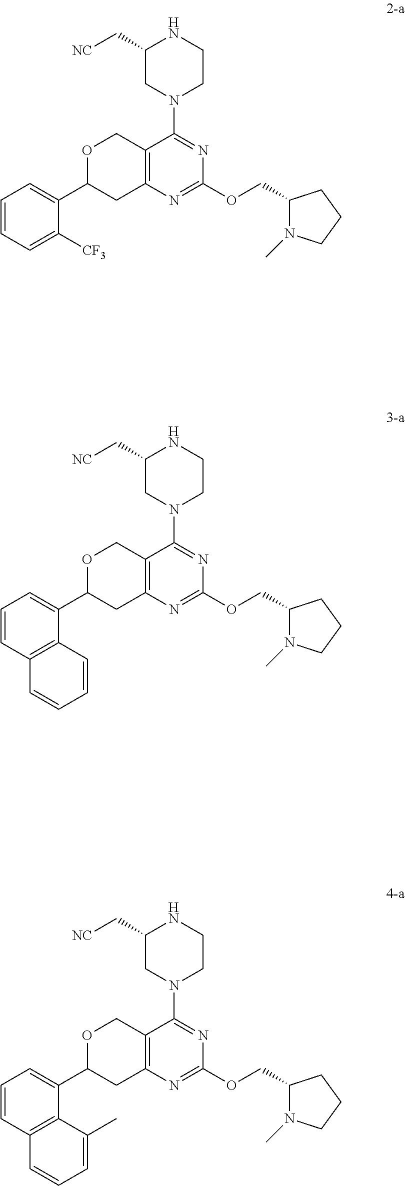 [SMILES: CN1CCC[C@H]1COc1nc2c(c(N3CCN[C@@H](CC#N)C3)n1)COC(c1ccccc1C(F)(F)F)C2 ; CN1CCC[C@H]1COc1nc2c(c(N3CCN[C@@H](CC#N)C3)n1)COC(c1cccc3ccccc13)C2 ; Cc1cccc2cccc(C3Cc4nc(OC[C@@H]5CCCN5C)nc(N5CCN[C@@H](CC#N)C5)c4CO3)c12]

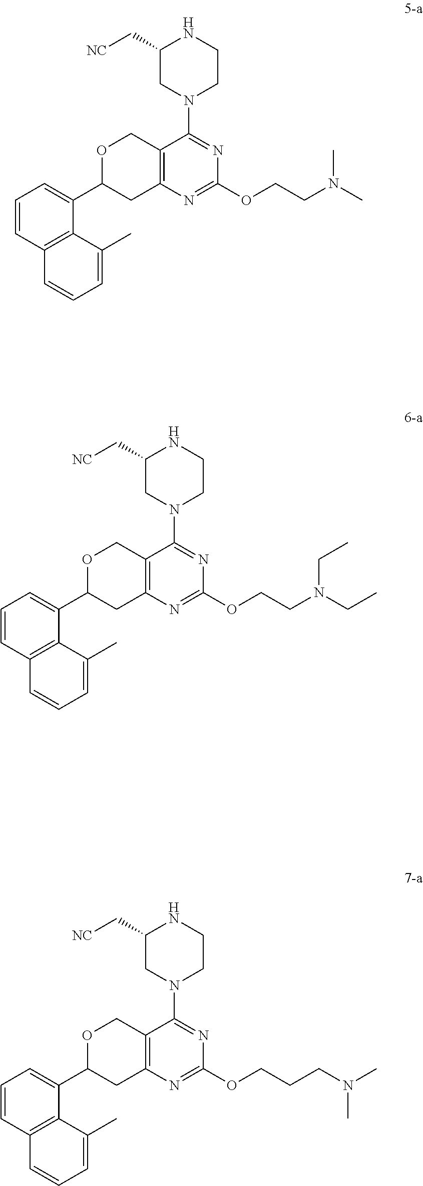 [SMILES: Cc1cccc2cccc(C3Cc4nc(OCCN(C)C)nc(N5CCN[C@@H](CC#N)C5)c4CO3)c12 ; CCN(CC)CCOc1nc2c(c(N3CCN[C@@H](CC#N)C3)n1)COC(c1cccc3cccc(C)c13)C2 ; Cc1cccc2cccc(C3Cc4nc(OCCCN(C)C)nc(N5CCN[C@@H](CC#N)C5)c4CO3)c12]

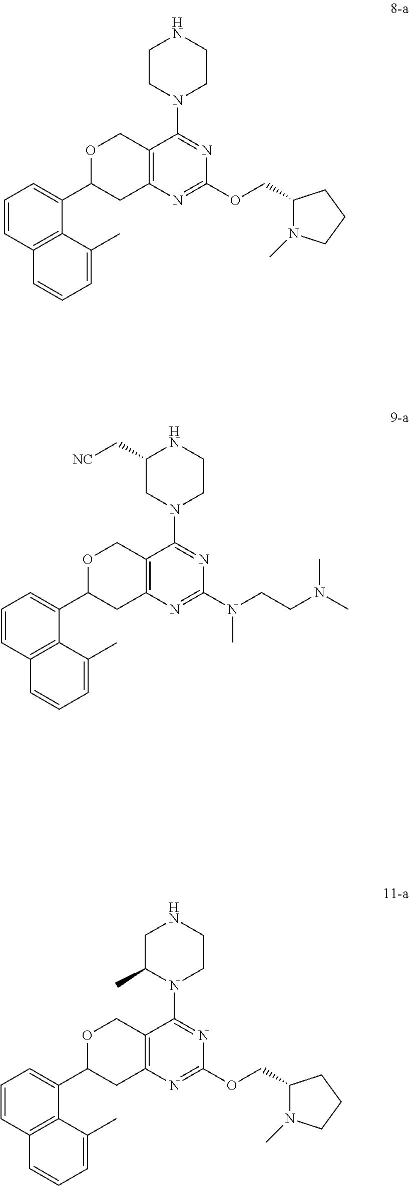 [SMILES: Cc1cccc2cccc(C3Cc4nc(OC[C@@H]5CCCN5C)nc(N5CCNCC5)c4CO3)c12 ; Cc1cccc2cccc(C3Cc4nc(N(C)CCN(C)C)nc(N5CCN[C@@H](CC#N)C5)c4CO3)c12 ; Cc1cccc2cccc(C3Cc4nc(OC[C@@H]5CCCN5C)nc(N5CCNC[C@@H]5C)c4CO3)c12]

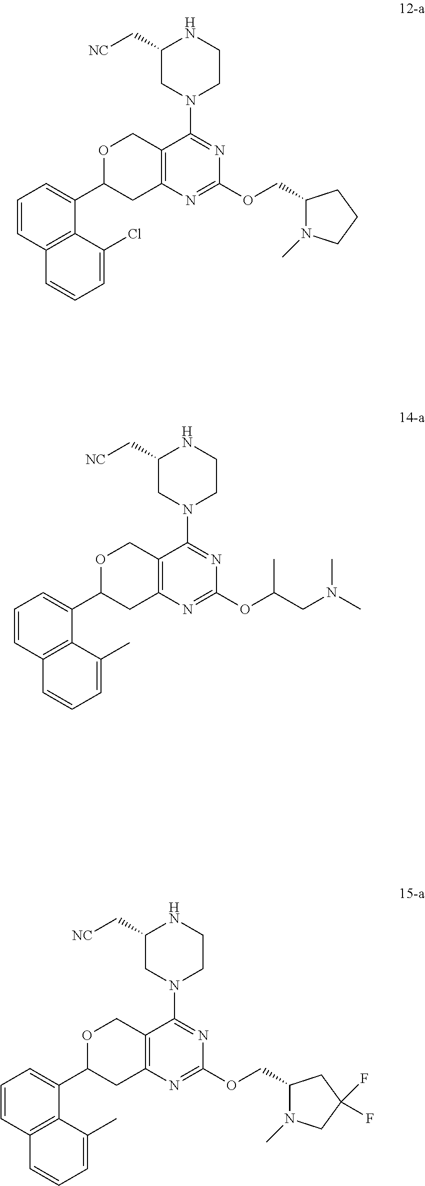 [SMILES: CN1CCC[C@H]1COc1nc2c(c(N3CCN[C@@H](CC#N)C3)n1)COC(c1cccc3cccc(Cl)c13)C2 ; Cc1cccc2cccc(C3Cc4nc(OC(C)CN(C)C)nc(N5CCN[C@@H](CC#N)C5)c4CO3)c12 ; Cc1cccc2cccc(C3Cc4nc(OC[C@@H]5CC(F)(F)CN5C)nc(N5CCN[C@@H](CC#N)C5)c4CO3)c12]

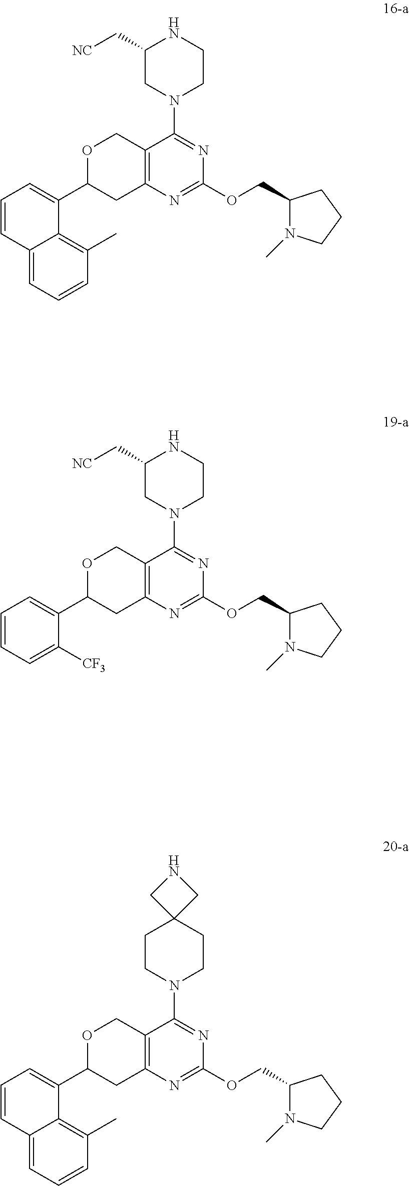 [SMILES: Cc1cccc2cccc(C3Cc4nc(OC[C@H]5CCCN5C)nc(N5CCN[C@@H](CC#N)C5)c4CO3)c12 ; CN1CCC[C@@H]1COc1nc2c(c(N3CCN[C@@H](CC#N)C3)n1)COC(c1ccccc1C(F)(F)F)C2 ; Cc1cccc2cccc(C3Cc4nc(OC[C@@H]5CCCN5C)nc(N5CCC6(CC5)CNC6)c4CO3)c12]

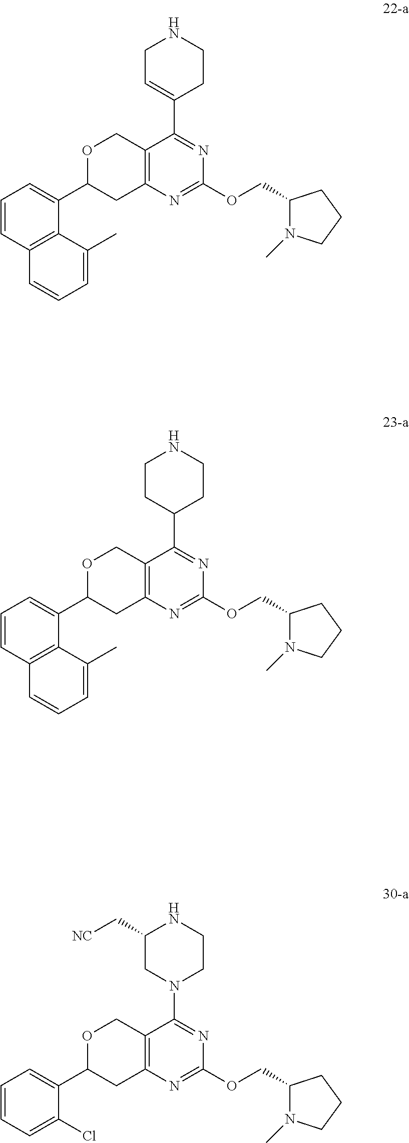 [SMILES: Cc1cccc2cccc(C3Cc4nc(OC[C@@H]5CCCN5C)nc(C5=CCNCC5)c4CO3)c12 ; Cc1cccc2cccc(C3Cc4nc(OC[C@@H]5CCCN5C)nc(C5CCNCC5)c4CO3)c12 ; CN1CCC[C@H]1COc1nc2c(c(N3CCN[C@@H](CC#N)C3)n1)COC(c1ccccc1Cl)C2]

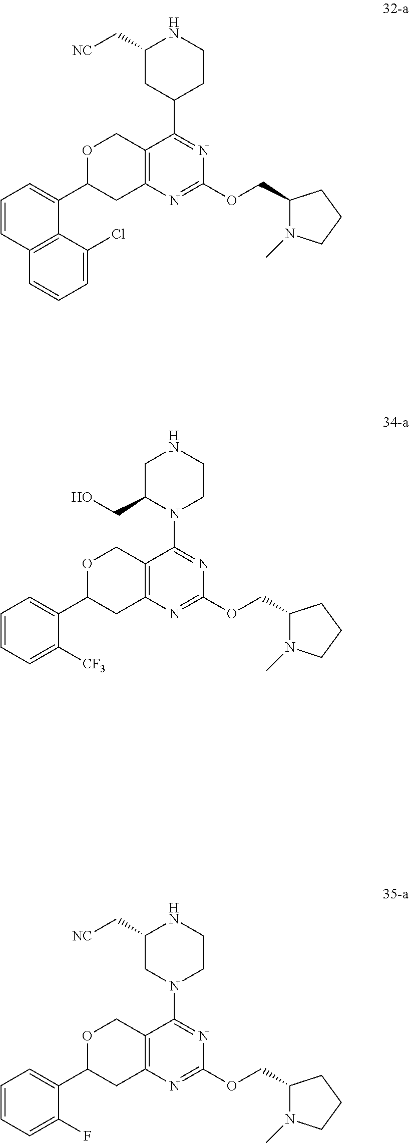 [SMILES: CN1CCC[C@@H]1COc1nc2c(c(C3CCN[C@@H](CC#N)C3)n1)COC(c1cccc3cccc(Cl)c13)C2 ; CN1CCC[C@H]1COc1nc2c(c(N3CCNC[C@@H]3CO)n1)COC(c1ccccc1C(F)(F)F)C2 ; CN1CCC[C@H]1COc1nc2c(c(N3CCN[C@@H](CC#N)C3)n1)COC(c1ccccc1F)C2]

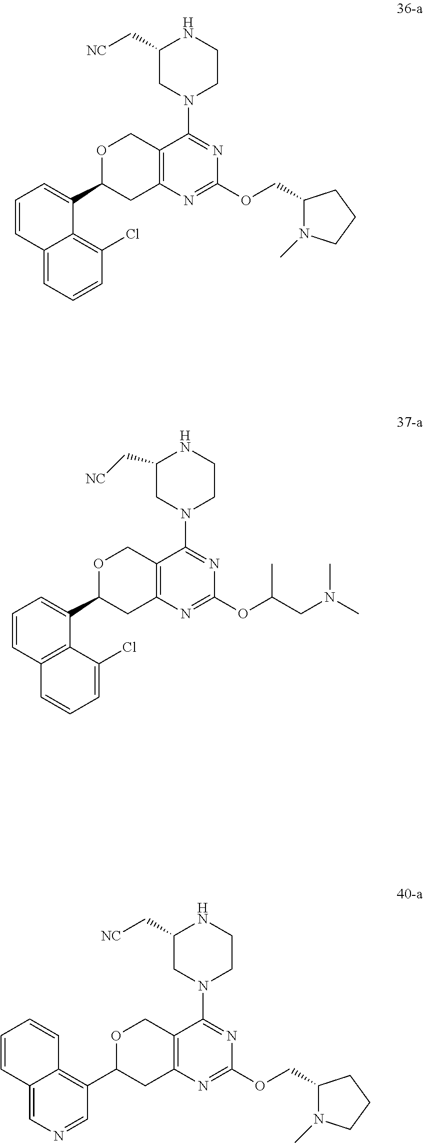 [SMILES: CN1CCC[C@H]1COc1nc2c(c(N3CCN[C@@H](CC#N)C3)n1)CO[C@H](c1cccc3cccc(Cl)c13)C2 ; CC(CN(C)C)Oc1nc2c(c(N3CCN[C@@H](CC#N)C3)n1)CO[C@H](c1cccc3cccc(Cl)c13)C2 ; CN1CCC[C@H]1COc1nc2c(c(N3CCN[C@@H](CC#N)C3)n1)COC(c1cncc3ccccc13)C2]

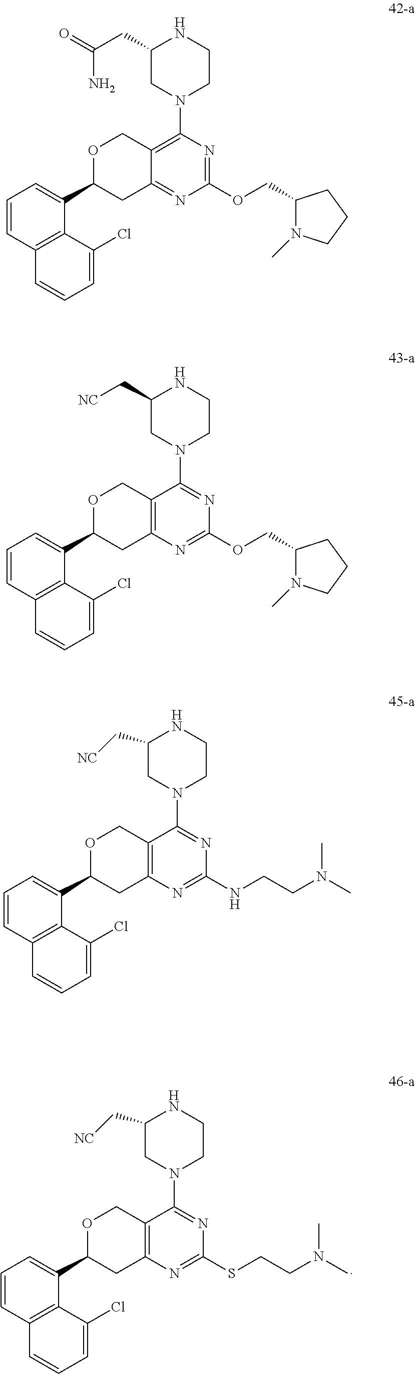 [SMILES: CN1CCC[C@H]1COc1nc2c(c(N3CCN[C@@H](CC(N)=O)C3)n1)CO[C@H](c1cccc3cccc(Cl)c13)C2 ; CN1CCC[C@H]1COc1nc2c(c(N3CCN[C@H](CC#N)C3)n1)CO[C@H](c1cccc3cccc(Cl)c13)C2 ; CN(C)CCNc1nc2c(c(N3CCN[C@@H](CC#N)C3)n1)CO[C@H](c1cccc3cccc(Cl)c13)C2 ; CN(C)CCSc1nc2c(c(N3CCN[C@@H](CC#N)C3)n1)CO[C@H](c1cccc3cccc(Cl)c13)C2]

The present disclosure also provides a pharmaceutical composition, comprising substance A and a pharmaceutical adjuvant: the substance A is the above-mentioned oxygen-containing heterocyclic compound represented by formula I, a pharmaceutically acceptable salt thereof, a solvate thereof, a solvate of the pharmaceutically acceptable salt thereof, a crystal form thereof or an isotopic compound thereof.

The present disclosure also provides use of substance A in the preparation of an RAS inhibitor, wherein the substance A is the above-mentioned oxygen-containing heterocyclic compound represented by formula I, a pharmaceutically acceptable salt thereof, a solvate thereof, a solvate of the pharmaceutically acceptable salt thereof, a crystal form thereof or an isotopic compound thereof.

The RAS comprises, for example, KRAS G12C. HRAS G12C or NRAS G12C mutation; for example, KRAS G12C.

The present disclosure also provides use of substance A in the preparation of a medicament, wherein the medicament is used to treat or prevent an RAS-mediated disease;

The substance A is the above-mentioned oxygen-containing heterocyclic compound represented by formula I, a pharmaceutically acceptable salt thereof, a solvate thereof, a solvate of the pharmaceutically acceptable salt thereof, a crystal form thereof or an isotopic compound thereof.

The RAS comprises, for example, KRAS G12C, HRAS G12C or NRAS G12C mutation; for example, KRAS G12C.

The RAS-mediated disease is, for example, cancer: the cancer is, for example, one or more of colon cancer, pancreatic cancer, breast cancer, prostate cancer, lung cancer, brain cancer, ovarian cancer, cervical cancer, testicular cancer, renal carcinoma, head or neck cancer, bone cancer, skin cancer, rectal cancer, liver cancer, colon cancer, esophageal cancer, gastric cancer, pancreatic cancer, thyroid cancer, bladder cancer, lymphoma, leukemia and melanoma.

The present disclosure also provides use of substance A in the preparation of a medicament, wherein the medicament is used to treat or prevent cancer;

the substance A is the above-mentioned oxygen-containing heterocyclic compound represented by formula I, a pharmaceutically acceptable salt thereof, a solvate thereof, a solvate of the pharmaceutically acceptable salt thereof, a crystal form thereof or an isotopic compound thereof.

The cancer is, for example, one or more of colon cancer, pancreatic cancer, breast cancer, prostate cancer, lung cancer, brain cancer, ovarian cancer, cervical cancer, testicular cancer, renal carcinoma, head or neck cancer, bone cancer, skin cancer, rectal cancer, liver cancer, colon cancer, esophageal cancer, gastric cancer, pancreatic cancer, thyroid cancer, bladder cancer, lymphoma, leukemia and melanoma.

The present disclosure also provides a method for inhibiting RAS, the method comprising administrating to a patient an therapeutically effective amount of substance A;

the substance A is the above-mentioned oxygen-containing heterocyclic compound represented by formula I, a pharmaceutically acceptable salt thereof, a solvate thereof, a solvate of the pharmaceutically acceptable salt thereof, a crystal form thereof or an isotopic compound thereof.

The RAS comprises, for example, KRAS G12C, HRAS G12C or NRAS G12C mutation; for example, KRAS G12C.

The present disclosure also provides a method for treating or preventing an RAS-mediated disease, the method comprising administrating to a patient an therapeutically effective amount of substance A;

the substance A is the above-mentioned oxygen-containing heterocyclic compound represented by formula I, a pharmaceutically acceptable salt thereof, a solvate thereof, a solvate of the pharmaceutically acceptable salt thereof, a crystal form thereof or an isotopic compound thereof.

The RAS comprises, for example, KRAS G12C. HRAS G12C or NRAS G12C mutation; for example, KRAS G12C.

The RAS-mediated disease is, for example, cancer: the cancer is, for example, one or more of colon cancer, pancreatic cancer, breast cancer, prostate cancer, lung cancer, brain cancer, ovarian cancer, cervical cancer, testicular cancer, renal carcinoma, head or neck cancer, bone cancer, skin cancer, rectal cancer, liver cancer, colon cancer, esophageal cancer, gastric cancer, pancreatic cancer, thyroid cancer, bladder cancer, lymphoma, leukemia and melanoma.

The present disclosure also provides a method for treating or preventing cancer, the method comprising administrating to a patient an therapeutically effective amount of substance A;

the substance A is the above-mentioned oxygen-containing heterocyclic compound represented by formula I, a pharmaceutically acceptable salt thereof, a solvate thereof, a solvate of the pharmaceutically acceptable salt thereof, a crystal form thereof or an isotopic compound thereof.

The cancer is, for example, one or more of colon cancer, pancreatic cancer, breast cancer, prostate cancer, lung cancer, brain cancer, ovarian cancer, cervical cancer, testicular cancer, renal carcinoma, head or neck cancer, bone cancer, skin cancer, rectal cancer, liver cancer, colon cancer, esophageal cancer, gastric cancer, pancreatic cancer, thyroid cancer, bladder cancer, lymphoma, leukemia and melanoma.

The term “more” refers to 2, 3, 4 or 5.

The term “pharmaceutically acceptable salt” refers to a salt prepared from compounds of the present disclosure with relatively non-toxic, pharmaceutically acceptable acids or bases. When compounds of the present disclosure contain relatively acidic functional groups, base addition salts can be obtained by contacting the neutral form of such compounds with a sufficient amount of pharmaceutically acceptable bases, either in pure solution or a suitable inert solvent. The pharmaceutically acceptable base addition salts include but are not limited to: lithium salt, sodium salt, potassium salt, calcium salt, aluminum salt, magnesium salt, zinc salt, bismuth salt, ammonium salt and diethanolamine salt. When compounds of the present disclosure contain relatively basic functional groups, acid addition salts can be obtained by contacting the neutral form of such compounds with a sufficient amount of pharmaceutically acceptable acids, either in pure solution or a suitable inert solvent. The pharmaceutically acceptable acids include inorganic acids, and the inorganic acids include but are not limited to: hydrochloric acid, hydrobromic acid, hydroiodic acid, nitric acid, carbonic acid, phosphoric acid, phosphorous acid and sulfuric acid. The pharmaceutically acceptable acids include organic acids, and the organic acids include but are not limited to: acetic acid, propionic acid, oxalic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, salicylic acid, tartaric acid, methanesulfonic acid, isonicotinic acid, acidic citric acid, oleic acid, tannic acid, pantothenic acid, hydrogen tartrate, ascorbic acid, gentisic acid, fumaric acid, gluconic acid, saccharic acid, formic acid, ethanesulfonic acid, pamoic acid (i.e., 4,4′-methylene-bis(3-hydroxy-2-naphthoic acid)) and amino acid (such as glutamic acid and arginine). When compounds of the present disclosure contain relatively acidic functional groups and relatively basic functional groups, such compounds can be converted into base addition salts or acid addition salts. For details, reference can be made to Berge et al., “Pharmaceutical Salts”, Journal of Pharmaceutical Science 66: 1-19 (1977), or Handbook of Pharmaceutical Salts: Properties, Selection, and Use (P. Heinrich Stahl and Camille G. Wermuth, ed., Wiley-VCH, 2002).

The term “solvate” refers to substance formed by combining compounds of the present disclosure with stoichiometric or non-stoichiometric solvents. The solvent molecules in the solvate may be present in a regular arrangement or a non-ordered arrangement. The solvents include but are not limited to: water, methanol and ethanol.

With regard to term “solvate of a pharmaceutically acceptable salt”, the “pharmaceutically acceptable salt” and “solvate” as described above refer to: 1, substance prepared by compounds of the present disclosure and relatively non-toxic, pharmaceutically acceptable acids or bases; and 2, substance formed by combining compounds of the present disclosure with stoichiometric or non-stoichiometric solvents. The “solvate of a pharmaceutically acceptable salt” includes, but is not limited to, the hydrochloric acid monohydrate of compounds of the present disclosure.

The term “stereoisomer” refers to an isomer in which the atoms or atomic groups in a molecule have the same interconnection order but different spatial arrangements, such as cis-trans isomers, optical isomers or atropisomers. These stereoisomers can be separated, purified and enriched by means of asymmetric synthesis methods or chiral separation methods (including but not limited to thin layer chromatography, rotation chromatography, column chromatography, gas chromatography and high-pressure liquid chromatography) or can also be obtained by means of chiral resolution via forming bonds (chemical bonding, etc.) or forming salts (physical bonding) with other chiral compounds, etc.

The tens “tautomer” refers to a functional group isomer resulting from the rapid movement of an atom in two positions in a molecule. For example, acetone and 1-propene-2-ol can be converted into each other by the rapid movement of hydrogen atoms on oxygen and α-carbon.

The term “crystal form” refers to substance in which ions or molecules are arranged strictly and periodically in a three-dimensional space according to a certain way and are repeated regularly and periodically at a certain interval: and since the periodic arrangements are different, there can be multiple crystal forms, which is also known as polymorphism.

The term “isotopic compound” refers to a compound in which one or more atoms are substituted with one or more atoms having a specific atomic mass or mass number. Examples of isotopes that can be incorporated into compounds of the present disclosure include, but are not limited to, isotopes of hydrogen, carbon, nitrogen, oxygen, fluorine, sulfur and chlorine (e.g., 2H, 3H, 13C, 14C, 15N, 18O, 17O, 18F, 35S and 36Cl). The isotopic compounds of the present disclosure can generally be prepared by substituting non-isotopically-labeled reagents with isotopically-labeled reagents according to the methods described herein.

When any variable (such as R¹⁻⁶) appears multiple times in the definition of a compound, the definition of the variable in each position is irrespective of that in the other positions, and these definitions of the variable are independent from and do not interfere with each other. Therefore, if a group is substituted with one, two or three R¹⁻⁶ groups, the group may be substituted with up to three R¹⁻⁶ groups, and the definition of R¹⁻⁶ in this position is independent from that in the other positions. In addition, a combination of substituents and for variables are only allowed if the combination produces a stable compound.

The term “halogen” refers to fluorine, chlorine, bromine or iodine.

The term “alkyl” refers to straight or branched alkyl having specified number of carbon atoms. Examples of alkyl include methyl, ethyl, n-propyl, isopropyl, n-butyl, tert-butyl, isobutyl, sec-butyl, n-pentyl, n-hexyl, n-heptyl, n-octyl and similar alkyl groups.

The term “alkoxy” refers to group —O—R^{X}, wherein R^{X} is alkyl as defined above.

The term “cycloalkyl” refers to saturated monocyclic alkyl, preferably saturated monocyclic alkyl having 3-7 ring carbon atoms, more preferably 3-6 carbon atoms, for example, cyclopropyl, cyclobutyl, cyclopentyl or cyclohexyl.

The term “heterocycloalkyl” refers to a saturated monocyclic group having heteroatoms, preferably a 3-7 membered saturated monocyclic group containing one, two or three ring heteroatoms independently selected from N, O or S. Examples of heterocycloalkyl include: pyrrolidinyl, tetrahydrofuryl, tetrahydropyranyl, tetrahydrothienyl, tetrahydropyridyl, tetrahydropyrrolyl, azetidinyl, thiazolidinyl, oxazolidinyl, piperidinyl, morpholinyl, thiomorpholinyl, piperazinyl, azepanyl, diazepanyl, oxazepanyl, etc. Preferred heterocyclyl is morpholin-4-yl, piperidin-1-yl, pyrolidin-1-yl, thiomorpholin-4-yl and 1,1-dioxo-thiomorpholin-4-yl.

The term “saturated heterocyclic ring” refers to a saturated cyclic group having heteroatoms and can be a monocyclic ring, a bridged ring or a spiro ring. The monocyclic “saturated heterocyclic ring” is the “heterocycloalkyl” as described above.

The term “partly saturated heterocyclic ring” refers to a partially saturated cyclic group having heteroatoms, which is neither fully saturated nor aromatic and can be a monocyclic ring, a bridged ring or a spiro ring. Examples of “partly saturated heterocyclic ring” include: pyranoid ring and 1,2,5,6-tetrahydropyridine.

The term “aryl” refers to an aromatic group composed of carbon atoms, in which each ring is aromatic, and examples include phenyl or naphthyl.

The term “heteroaryl” refers to an aromatic group containing heteroatoms, in which each ring is aromatic: preferred heteroaryl is an aromatic 5-6 membered monocyclic ring or 9-10 membered bicyclic ring containing 1, 2 or 3 heteroatoms independently selected from nitrogen, oxygen or sulfur, for example, furyl, pyridyl, pyridazinyl, pyrimidinyl, pyrazinyl, thienyl, isoxazolyl, oxazolyl, diazolyl, imidazolyl, pyrrolyl, pyrazolyl, triazolyl, tetrazolyl, thiazolyl, isothiazolyl, thiadiazolyl, benzoimidazolyl, indolyl, indazolyl, benzothiazolyl, benzoisothiazolyl, benzoxazolyl, benzoisoxazolyl, quinolyl, isoquinolyl, etc.

The term “pharmaceutical adjuvant” refers to excipients and additives used in the production of drugs and formulation of prescriptions, and is all substances contained in a pharmaceutical preparation except for active ingredients. Reference can be made to Pharmacopoeia of the People's Republic of China (2015 edition, four volumes), or Handbook of Pharmaceutical Excipients (Raymond C Rowe, 2009 Sixth Edition).

The term “treat/treating/treatment” refers to therapeutic therapy. With regard to specific conditions. “treat/treating/treatment” refers to: (1) alleviating one or more biological manifestations of a disease or condition; (2) interfering with (a) one or more points in the biological cascade resulting from or caused by a condition or (b) one or more of biological manifestations of a condition: (3) improving one or more symptoms, impacts or side effects related to a condition, or one or more symptoms, impacts or side effects related to a condition or the treatment thereof: or (4) alleviating a condition or one or more biological manifestations of the condition.

The term “prevent/preventing/prevention” refers to a reduction in the risk of acquiring or developing a disease or disorder.

The term “therapeutically effective amount” refers to an amount of a compound that is sufficient to effectively treat the diseases or conditions described herein when administered to a patient. The “therapeutically effective amount” will vary according to compounds, conditions and severity thereof, and age of patients to be treated, but can be adjusted by those of skill in the art as needed.

The term “patient” refers to any animal, preferably mammal, most preferably human, which is about to receive or has received the compound or composition according to examples of the present disclosure. The term “mammal” includes any mammal. Examples of mammals include, but are not limited to, cattle, horses, sheep, pigs, cats, dogs, mice, rats, rabbits, guinea pigs, monkeys, humans, etc., most preferably humans.

On the basis of not departing from common knowledge in the art, the above-mentioned various preferred conditions can be combined in any manner, such that various preferred examples of the present disclosure are obtained.

Reagents and starting materials used in the present disclosure are all commercially available.

In the present disclosure, the room temperature refers to the ambient temperature, which is 10° C., to 35° C.

The positive effect of the present disclosure lies in: the oxygen-containing heterocyclic compound is expected to treat and/or prevent various Ras-mediated diseases.

### DETAILED DESCRIPTION OF THE PREFERRED EMBODIMENT

The present disclosure is further illustrated by the following examples, but the present disclosure is not limited thereto. Experimental methods with specific conditions are not indicated in the following examples, but can be chosen according to conventional methods and conditions or commodity instructions.

In the present disclosure, the room temperature refers to the ambient temperature, which is 10° C., to 35° C. Overnight refers to 8 to 15 hours. Reflux refers to the reflux temperature of the solvent under normal pressure.

The following is a list of abbreviations used in the examples;

DMF N,N-dimethylformamide

HATU 2-(7-azobenzotriazole)-tetramethylurea hexafluorophosphate

EDCI 1-(3-dimethylaminopropyl)-3-ethylcarbodiimide hydrochloride

DIPEA diisopropylethylamine

Pd(PPh₃)₄ palladium tetraphenylphosphine

Pd(dppf)Cl₂ [1,1′-bis(diphenylphosphino)ferrocene]palladium dichloride dichloromethane complex

LiHMDSbis-(trimethylsilyl) lithium amide

MCPBA m-chloroperoxybenzoic acid

### Example 1 Synthetic Route of Compound 1

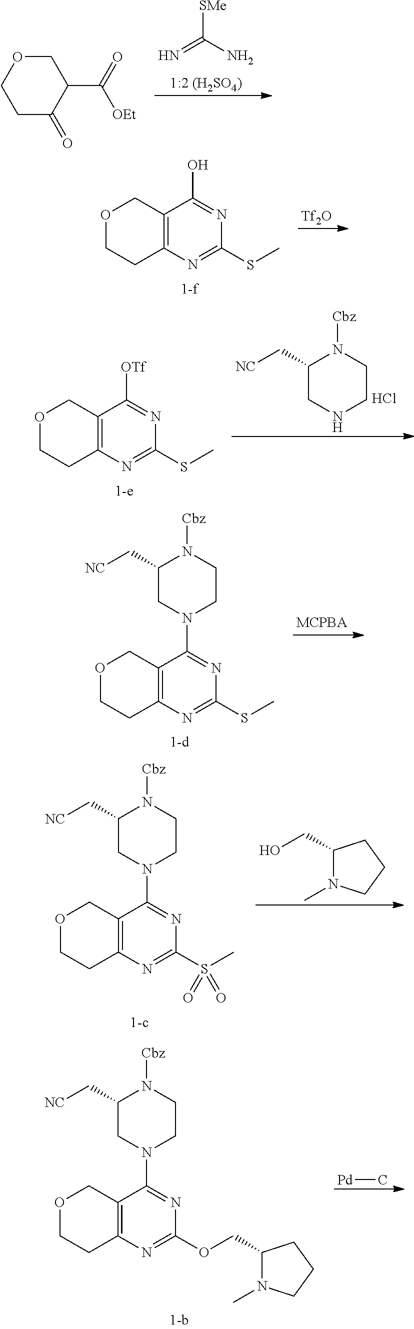

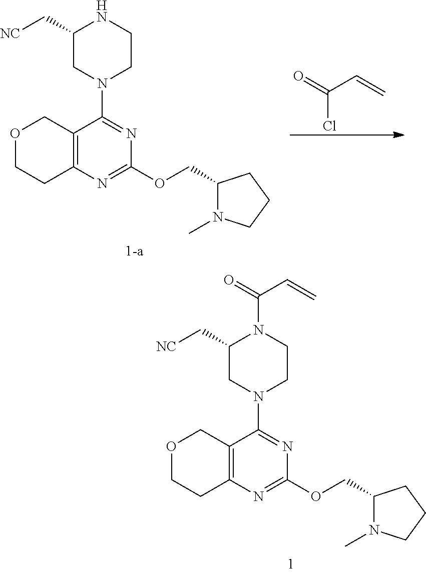

### Synthesis of Compound 1-f

In an ice-water bath, to a solution of compound ethyl 4-carbonyl-tetrahydropyran-3-carboxylate (1 g, 5.81 mmol) in methanol (20 mL) were added 2-methyl-2-thiourea sulfate (1.45 g, 10.43 mmol) and sodium methylate (1.57 g, 29.07 mmol) respectively. After completion of the addition, the reaction mixture was stirred at room temperature for 3 hours. LCMS monitoring indicated that the reaction was incomplete, and by-products were produced: the pH was adjusted to 5 with 1 M dilute hydrochloric acid: 30 mL of water and 30 mL of ethyl acetate were respectively added, and the mixture was stirred for 10 minutes. The mixture was filtered to give compound 1-f (684 mg, 59%) as a white solid. LC-MS (ESI): m/z=199.1 [M+H]⁺.

### Synthesis of Compound 1-e

At room temperature, 1-f (684 mg, 3.45 mmol) was dissolved in dichloromethane (20 mL), and DIPEA (1.14 mL, 6.91 mmol) was added. Under ice-water bath cooling, trifluoroinethanesulfonic anhydride (0.871 mL, 5.18 mmol) was added dropwise with stirring. After completion of the dropwise addition, the mixture was continuously stirred in an ice-water bath for 1 hour. LCMS monitoring indicated that the reaction was complete; the reaction was then quenched with a saturated sodium bicarbonate solution, extracted with dichloromethane (30 mL*4), dried, concentrated and subjected to a silica gel column to give compound 1-e (996 mg, 87%) as a light brown oil. LC-MS (ESI): m/z=331.2 [M+H]⁻.

### Synthesis of Compound 1-d

At room temperature, 1-e (400 mg, 1.21 mmol) was dissolved in DMF (15 mL), and compound benzyl (S)-2-cyanomethylpiperazine-1-carboxylate hydrochloride (429 mg, 1.45 mmol) and DIPEA (0.6 mL, 3.64 mmol) were respectively added. The reaction mixture was replaced three times with nitrogen and heated to 100° C., and stirred for 1 hour under nitrogen protection. LCMS monitoring indicated that the reaction was complete, and the reaction mixture was cooled to room temperature, quenched with a saturated sodium bicarbonate solution and extracted with ethyl acetate (50 mL*3). The organic layer was washed three times with saturated brine, dried, concentrated and subjected to a silica gel column to give compound 1-d (522 mg, 98%) as a white solid. LC-MS (ESI): m/z=440.4 [M+H]⁺.

### Synthesis of Compound 1-c

At room temperature, 1-d (522 mg, 1.19 mmol) was dissolved in ethyl acetate (30 mL), and MCPBA (601.5 mg, 2.97 mmol) was added. After completion of the addition, the reaction mixture was stirred at room temperature for 1 hour. TLC monitoring indicated that the reaction was complete; the reaction was then quenched with a saturated sodium bicarbonate solution, extracted with ethyl acetate (50 mL*2), dried, concentrated and subjected to a silica gel column to give compound 1-c (552 mg, 99%) as a white solid. LC-MS (ESI): m/z=472.4 [M+H]⁺.

### Synthesis of Compound 1-b

1-c (552 mg, 1.17 mmol) was dissolved in toluene (15 mL): under ice-water bath cooling. N-methyl-L-prolinol (243.7 μL, 2.05 mmol) and sodium tert-butoxide (225 mg, 2.34 mmol) were respectively added. After completion of the addition, under nitrogen protection, the mixture was continuously stirred in an ice-water bath for 30 minutes. TLC monitoring indicated that the reaction was complete; the reaction was then quenched with water, extracted twice with ethyl acetate, dried, concentrated and subjected to a silica gel column to give compound 1-b (443 mg, 75%) as a white solid. LC-MS (ESI): m/z=507.5 [M+H]⁺.

### Synthesis of Compound 1-a

1-b (150 mg, 0.296 mmol) was dissolved in ethyl acetate (30 mL), and 10% palladium-carbon (450 mg) was added: after replaced three times with hydrogen, the reaction mixture was stirred at room temperature under hydrogen atmosphere for 3 hours. TLC monitoring indicated that the reaction was complete; the product was filtered through diatomite, rinsed with methanol and subjected to rotary evaporation to give 1-a (74 mg, 67%) as a white solid, which was used directly in the next step without further purification. LC00000000000000000000000000000b-MS (ESI): m/z=373.4 [M+H]⁺.

### Synthesis of Compound 1

At room temperature, to a solution of 1-a (74 mg, 0.199 mmol) in dichloromethane (30 mL) were added DIPEA (164 μL, 0.995 mmol) and acryloyl chloride (24 μL, 0.298 mmol) respectively. Under nitrogen atmosphere, at room temperature, the reaction mixture was reacted overnight: LCMS monitoring indicated that the reaction was complete; the reaction was then quenched with a saturated sodium bicarbonate solution and extracted with dichloromethane (30 mL×3). The organic phase was dried, subjected to rotary evaporation and purified by preparative TLC (DCM:MeOH=10:1) to give compound 1 (20 mg, 24%) as a white solid. LC-MS (ESI): m/z=427.2 [M+H]⁺: ¹H NMR (400 MHz, CDCl₃); δ 6.65-6.50 (m, 1H), 6.41-6.24 (m, 1H), 6.16-5.69 (m, 1H), 4.78-4.67 (m, 1H), 4.67-4.52 (m, 2H), 4.38 (dd, 1H, J=11.6 Hz, J=4.8 Hz), 4.12-3.93 (m, 3H), 3.59-3.48 (m, 2H), 3.36 (dd, 2H, J=14 Hz, J=3.6 Hz), 3.26-3.00 (m, 4H), 2.95-2.81 (m, 3H), 2.77 (s, 3H), 2.73-2.58 (m, 2H), 2.26-1.88 (m, 4H).

### Example 2 Synthetic Route of Compound 2

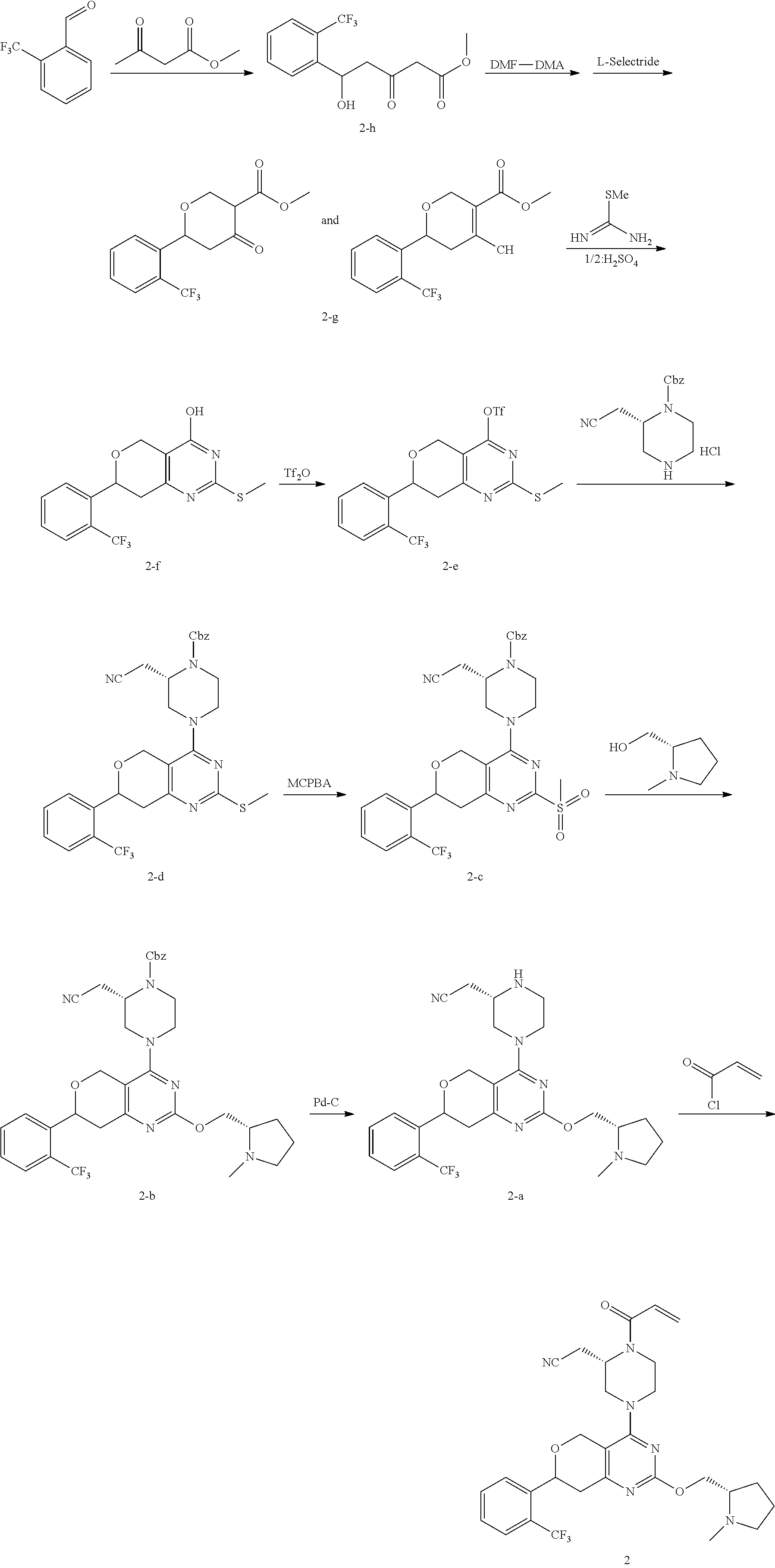

### Synthesis of Compound 2-h

At room temperature, NaH (60%, 3.6 g, 90.0 mmol) was added to THE (100 mL); under nitrogen atmosphere, methyl acetoacetate (10.4 g, 90.0 mmol) was added at room temperature. Under nitrogen atmosphere, the mixture was stirred for 30 minutes at room temperature, and then n-BuLi (2.5 M, 36 mL, 90.0 mmol) was added dropwise at −15° C., to −10° C.: after completion of the addition, the reaction mixture was kept at this temperature and stirred for 30 minutes, and a solution of 2-trifluoromethylbenzaldehyde (5.2 g, 29.9 mmol) in THE (10 mL) was added dropwise: after completion of the addition, the mixture was stirred at low temperature (−10° C., to 0° C.) for 2 hours. The reaction was quenched by adding a saturated ammonium chloride solution (100 mL) and extracted with ethyl acetate (100 mL*3); the organic phase was washed with saturated brine (100 mL), dried over anhydrous sodium sulfate, filtered and concentrated to give a crude product: the crude product was separated and purified through a flash column chromatography (EA/PE=1/3) to give compound 2-h (5.8 g, 67%) as a pale yellow liquid, which was used directly in the next reaction.

### Synthesis of Compound 2-g

Under nitrogen atmosphere, at room temperature, to a solution of compound 2-h (5.8 g, 20.0 mmol) in DCM (120 mL) was added DMF-DMA (3.2 mL, 24.1 mmol). After completion of the addition, the mixture was stirred at room temperature for 45 minutes. BF₃.Et₂O (3.2 mL, 25.4 mmol) was added and continuously stirred at room temperature for 1 hour. The reaction mixture was diluted by adding dichloromethane (200 washed successively with a saturated NaHCO₃ solution (400 mL) and saturated brine (200 mL), dried over anhydrous sodium sulfate, filtered and concentrated to give a crude product. The crude product was dissolved in THE (60 mL): at −78° C. under nitrogen atmosphere, a solution of lithium tri-sec-butyl borohydride in tetrahydrofuran (30.0 mL, 30.0 mmol) was added dropwise, and the mixture was stirred at this temperature for 1 hour. The reaction was quenched by adding saturated ammonium chloride (200 mL) and extracted with ethyl acetate (100 mL*3); the organic phase was washed with saturated brine (100 mL*2) and concentrated to give compound 2-g (3.8 g, 63%) as a yellow oil. LC-MS (ESI): m/z=303.1 [M+H]⁺.

### Synthesis of Compound 2-f

Under nitrogen atmosphere, in an ice-water bath, to 2-g (3.0 g, 10.0 mmol) in methanol (100 mL), sodium methylate (2.7 g, 50.0 mmol) and 2-methyl-2-thiourea sulfate (2.5 g, 8.4 mmol) were successively added. After completion of the addition, the mixture was warmed to room temperature and stirred overnight. The pH was adjusted to 5 with 1 M dilute hydrochloric acid, and the solid was precipitated out and filtered to give compound 2-f (1.7 g, 50%) as a pale yellow solid. LC-MS (ESI): m/z=343.0 [M+1]⁺.

### Synthesis of Compound 2-e

Under nitrogen atmosphere, in an ice-water bath, to 2-f (1.7 g, 5.0 mmol) in dichloromethane (40 mL). DIPEA (2.1 mL, 12.3 mmol) and trifluoromethanesulfonic anhydride (1.0 mL, 6.3 mmol) were successively added. After completion of the addition, the mixture was stirred at 0° C. for 2 hours. The reaction was quenched by adding saturated sodium bicarbonate solution (50 mL), extracted with DCM (50 mL*2) and concentrated to give compound 2-e (1.5 g) which was used directly in the next reaction. LC-MS (ESI): m/z=474.9 [M+H]⁺.

### Synthesis of Compound 2-d

At room temperature, compound 2-e (1.5 g, 3.2 mmol) was dissolved in DMF (10 mL), and DIPEA (0.9 mL, 5.6 mmol) and benzyl (S)-2-cyanomethylpiperazine-1-carboxylate hydrochloride (0.8 g, 2.8 mmol) were successively added. The mixture was stirred at 100° C. for 1 hour and then quenched by adding water (100 mL), extracted with ethyl acetate (80 mL*2) and concentrated: the crude product was separated and purified through a flash colon n chromatography (EA/PE=1/1) to give compound 2-d (0.93 g, 50%) as a white solid. LC-MS (ESI): m/z=584.0 [M+H]⁺.

### Synthesis of Compound 2-c

Compound 2-d (0.4 g, 0.69 mmol) was dissolved in ethyl acetate (20 mL), and MCPBA (0.23 g, 1.4 mmol) was added at room temperature. The mixture was stirred at room temperature for 1 hour and then quenched by adding saturated sodium bicarbonate solution (50 mL) and extracted with ethyl acetate (50 mL*2); the organic phase was concentrated, and the crude product was separated and purified through a flash column chromatography (DCM/MeOH=9/1) to give compound 2-c (0.33 g, 78%) as a white solid. LC-MS (ESI): m/z=616.0 [M+H]⁺.

### Synthesis of Compound 2-b

Under ice-water bath cooling, to compound 2-c (0.33 g, 0.54 mmol) in toluene (15 mL). N-methyl-L-prolinol (0.1 mL, 0.9 mmol) and t-BuONa (0.1 g, 0.9 mmol) were successively added. Under nitrogen atmosphere, the mixture was stirred in an ice-water bath for 0.5 hours and then quenched by adding water (10 mL) and extracted with ethyl acetate (30 mL*2); the organic phase was concentrated and the crude product was separated and purified through a flash column chromatography (MeOH/DCM=1/9) to give compound 2-b (0.26 g, 74%) as a white solid. LC-MS (ESI): m/z=651.3 [M+1]⁻.

### Synthesis of Compound 2-a

Compound 2-b (0.26 g, 0.4 mmol) in methanolic ammonia (7 M, 50 mL) was dissolved and cooled to −78° C. replaced twice with nitrogen and then added Pd/C (70 mg) and replaced three times with hydrogen. The reaction mixture was warmed to room temperature and stirred under hydrogen for 2 hours. The reaction mixture was filtered and concentrated to give compound 2-a (0.16 g, 77%). LC-MS (ESI): m/z=517.2 [M+1]⁺.

### Synthesis of Compound 2

At room temperature, compound 2-a (0.12 g, 0.23 mmol) was dissolved in DCM (10 mL): DIPEA (75 μL, 0.45 mmol) and acryloyl chloride (25 μL, 0.23 mmol) were successively added. Under nitrogen atmosphere, the mixture was stirred at room temperature for 20 hours and then quenched by adding water (10 mL) and extracted with DCM (50 mL*3). The organic phase was concentrated: the crude product was separated and purified through a flash column chromatography (MeOH/DCM=119) to give compound 2 (58 mg, 44%) as a white solid. LC-MS (ESI): m/z=571.3 [M+1]⁺: ¹H NMR (500 MHz, CD₃OD): δ 7.84 (d, 1H, J=7.5 Hz), 7.68-7.76 (m, 2H), 7.53 (t, 1H, J=7.5 Hz), 6.73-6.97 (m, 1H), 6.30 (d, 1H, J=16.5 Hz), 5.85 (d, 1H, J=9.5 Hz), 5.21 (t, 1H, J=11.0 Hz), 4.99-5.02 (m, 2H), 4.25-4.43 (m, 2H), 4.06-4.16 (m, 1H), 3.80-3.96 (m, 1H), 3.43-3.80 (m, 2H), 3.20-3.33 (m, 1H), 2.97-3.19 (m, 4H), 2.81-2.95 (m, 2H), 2.70-2.83 (m, 1H), 2.53 (d, 3H, J=4.5 Hz), 2.32-2.45 (m, 1H), 2.06-2.15 (m, 1H), 1.79-1.90 (m, 2H), 1.68-1.77 (m, 1H), 1.31-1.39 (m, 1H).

### Synthesis of Compounds 2-1 and 2-2

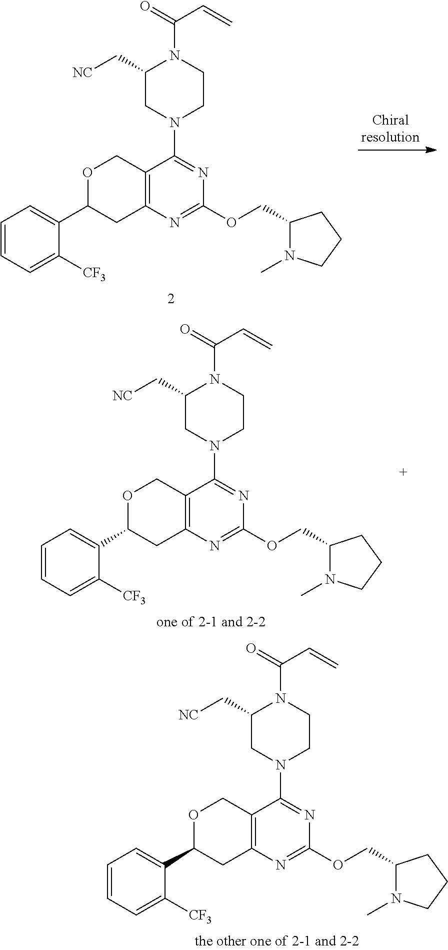 [SMILES: C=CC(=O)N1CCN(c2nc(OC[C@@H]3CCCN3C)nc3c2COC(c2ccccc2C(F)(F)F)C3)C[C@@H]1CC#N ; C=CC(=O)N1CCN(c2nc(OC[C@@H]3CCCN3C)nc3c2CO[C@@H](c2ccccc2C(F)(F)F)C3)C[C@@H]1CC#N ; C=CC(=O)N1CCN(c2nc(OC[C@@H]3CCCN3C)nc3c2CO[C@H](c2ccccc2C(F)(F)F)C3)C[C@@H]1CC#N]

Compound 2 (29 mg, 0.05 mmol) was purified by chiral resolution to give compound 2-1 (10 mg, 34%) as a white solid and compound 2-2 (10 mg, 34%) as a white solid.

| Chiral analysis conditions | Chiral preparation conditions |
|---|---|
| equipment: SFC Method Station | instrument: SFC-80 (Thar, Waters) |
| (Thar. Waters) | chromatographic column: OD 20 * |
| chromatographic column: OD-H | 250 mm. 10 μm (Daicel) |
| 4.6 * 100 mm, 5 μm (Daicel) | column temperature: 35° C. |
| column temperature: 40° C. | mobile phase: CO₂/MeOH (0.1% |
| mobile phase: CO₂/MeOH (0.1% | TEA) = 45/55 |
| TEA) = 65/35 | flow rate: 80 g/min |
| flow rate: 4.0 ml/min | back pressure: 100 bar |
| wavelength: 254 nm | detection wavelength: 214 nm |
| back pressure: 120 bar | cycling time: 6.0 min |
| | sample solution: 29 mg dissolved |
| | in 8 ml of methanol |
| 2-1: retention time: 0.92 min; | |
| d.e. % = 100.0%; | |
| 2-2: retention time: 2.74 min; | |
| d.e. % = 98.0%. | |

2-1: LC-MS (ESI): m/z=571.2 [M+1]⁺: ¹H NMR (400 MHz, CDCl₃): δ 7.75 (d, 1H, J=7.6 Hz), 7.67 (d, 1H, J=7.6 Hz), 7.63 (t, 1H, J=8.0 Hz), 7.44 (t, 1H, J=8.0 Hz), 6.54-6.65 (m, 1H), 6.38 (dd, 1H, J=16.4, 1.6 Hz), 5.83 (d, 1H, J=10.0 Hz), 5.14 (dd, 1H, J=10.8, 3.2 Hz), 4.88 (d, 1H, J=14.0 Hz), 4.80 (d, 1H, J=13.6 Hz), 4.51-5.12 (m, 2H), 4.37 (dd, 1H, J=10.8, 5.2 Hz), 4.18 (dd, 1H, J=10.8, 6.4 Hz), 3.84-4.10 (m, 1H), 3.70-3.83 (m, 1H), 3.32-3.64 (m, 1H), 2.75-3.27 (m, 6H), 2.63-2.72 (m, 1H), 2.48 (s, 3H), 2.25-2.32 (m, 1H), 1.98-2.10 (m, 1H), 1.69-1.90 (m, 3H), 1.28-1.39 (m, 1H).

2-2: LC-MS (ESI): m/z=571.2 [M+1]⁺: ¹H NMR (400 MHz, CDCl₃): δ 7.76 (d, 1H, J=7.6 Hz), 7.67 (d, 1H, J=7.6 Hz), 7.63 (t, 1H, J=7.6 Hz), 7.44 (t, 1H, J=7.6 Hz), 6.50-6.65 (m, 1H), 6.39 (dd, 1H, J=16.8, 1.6 Hz), 5.83 (d, 1H, J=11.2 Hz), 5.17 (dd, 1H, J=12.0, 4.0 Hz), 4.89 (d, 1H, J=14.0 Hz), 4.78 (d, 1H, J=13.2 Hz), 4.46-5.11 (m, 2H), 4.39 (dd, 1H, J=10.4, 4.8 Hz), 4.16 (dd, 1H, J=10.4, 6.4 Hz), 3.94-4.01 (m, 1H), 3.62-3.84 (m, 1H), 3.38-3.56 (m, 1H), 2.59-3.16 (m, 7H), 2.47 (s, 3H), 2.24-2.33 (m, 1H), 1.98-2.10 (m, 1H), 1.69-1.89 (m, 3H), 1.28-1.38 (m, 1H).

### Example 3 Synthetic Route of Compound 3

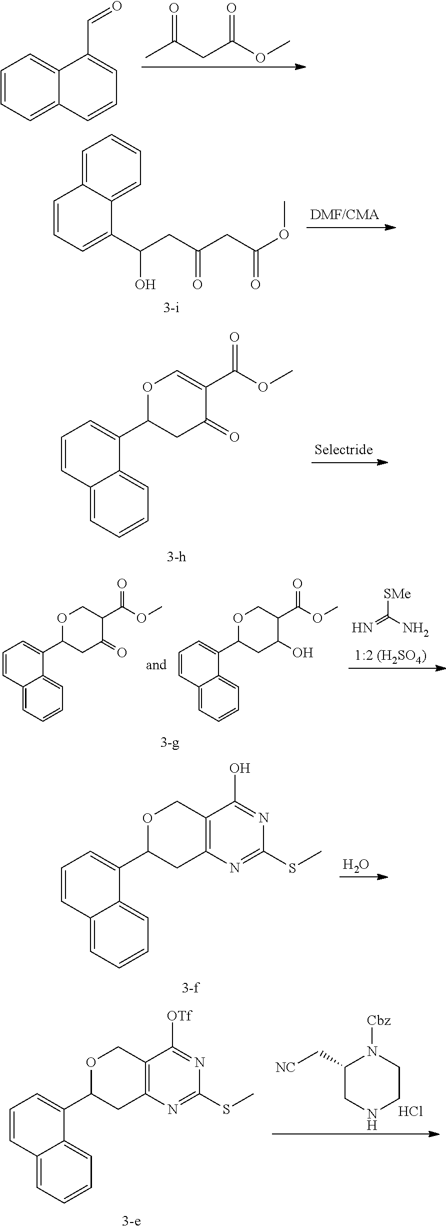

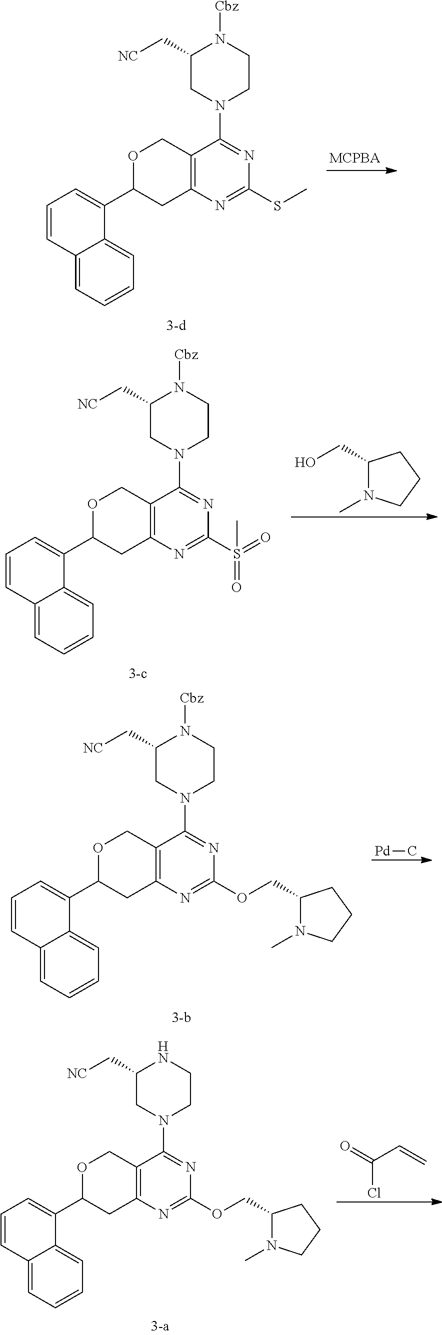

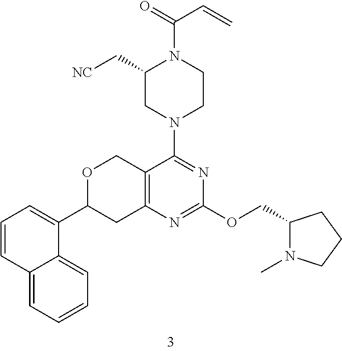 [SMILES: C=CC(=O)N1CCN(c2nc(OC[C@@H]3CCCN3C)nc3c2COC(c2cccc4ccccc24)C3)C[C@@H]1CC#N]

### Synthesis of Compound 3-i

At room temperature. NaH (60%, 3.0 g, 75.0 mmol) was added to THY (100 mL); under nitrogen atmosphere, methyl acetoacetate (8 mL, 77.0 mmol) was added at room temperature. Under nitrogen atmosphere, the mixture was stirred at room temperature for 30 minutes, and then n-BuLi (2.5 M, 30.8 mL, 77.0 mmol) was added dropwise at −15° C., to −10° C. After completion of the addition, the reaction mixture was kept at this temperature and stirred for 30 minutes, and then a solution of compound 1-naphthaldehyde (4.0 g, 25.6 mmol) THF (10 mL) was added dropwise. After completion of the addition, the mixture was stirred at low temperature (−10° C., to 0° C.) for 2 hours. The reaction was quenched by adding a saturated ammonium chloride solution (100 mL) and extracted with ethyl acetate (100 mL*3); the organic phase was washed with saturated brine (100 mL), dried over anhydrous sodium sulfate, filtered and concentrated to give a crude product: the crude product was separated and purified through a flash column chromatography (EA/PE=1/3) to give compound 3-i (4.5 g, 64%) as a pale yellow liquid.

### Synthesis of Compound 3-h

Compound 3-i (3.3 g, 12.1 mmol) was dissolved in DCM (120 mL), and under nitrogen atmosphere. DMF-DMA (1.6 mL, 12.0 mmol) was added at room temperature. The reaction mixture was stirred at room temperature for 45 minutes, and then BF₃.Et₂O (1.6 mL, 12.7 mmol) was added. The mixture was stirred at room temperature for 1 hour and then diluted with 200 mL of dichloromethane: the organic phase was washed successively with a saturated NaHCO₃ solution (400 mL) and saturated brine (200 mL), dried over anhydrous sodium sulfate, filtered and concentrated to give a crude product, which was separated and purified through a flash column chromatography (EA/PE=1/3) to give compound 3-h (3.0 g, 88%) as a pale yellow liquid. LC-MS (ESI): m/z=283.1 [M+H]⁺.

### Synthesis of Compound 3-g

At −78° C. under nitrogen atmosphere, to a solution of compound 3-h (2.3 g, 8.1 mmol) in THF (60 mL) was added dropwise a solution of lithium tri-sec-butyl borohydride un tetrahydrofuran (1 M, 8.3 mL, 8.3 mmol). After completion of the addition, the mixture was stirred at this temperature for 1 hour: the reaction was quenched by adding saturated ammonium chloride (20 mL) and extracted with ethyl acetate (100 mL*3); the organic phase was washed with saturated sodium chloride and concentrated to give a crude product, which was separated and purified through a flash column chromatography (EA/PE=1/4) to give compound 3-g (2.8 g) as a yellow oil. LC-MS (ESI): m/z=285.1 [M+1]⁻.

### Synthesis of Compound 3-f

In an ice-water bath, to a solution of compound 3-g (2.8 g, 10.0 mmol) in methanol (100 mL) were successively added sodium methylate (2.7 g, 50.0 mmol) and 2-methyl-2-thiourea sulfate (2.6 g, 8.8 mmol). After completion of the addition, the reaction mixture was warmed to room temperature and stirred overnight. The pH was adjusted to 5 with a 1 M hydrochloric acid solution: the solid was precipitated out, filtered, washed with water (50 mL*3) and dried to give crude product 3-f (1.3 g, 49% for two steps) as a pale yellow solid. LC-MS (ESI): m/z=325.0 [M+1]⁺.

### Synthesis of Compound 3-e

In an ice-water bath, to a solution of compound 3-f (0.65 g, 2.0 mmol) in DCM (40 mL) were successively added DIPEA (0.67 mL, 4.1 mmol) and trifluoromethanesulfonic anhydride (0.34 mL, 2.1 mmol). After completion of the addition, the mixture was stirred in an ice-water bath for 2 hours, quenched by adding a saturated sodium bicarbonate solution (50 mL) and extracted with DCM (50 mL*2); the organic phase was dried over anhydrous sodium sulfate, filtered and concentrated to give compound 3-e (0.62 g) which was used directly in the next reaction.

### Synthesis of Compound 3-d

At room temperature, compound 3-e (0.62 g, 1.4 mmol) was dissolved in DMF (10 mL), and DIPEA (0.45 mL, 2.8 mmol) and benzyl (S)-2-cyanomethylpiperazine-1-carboxylate hydrochloride (0.41 g, 1.4 mmol) were successively added. After completion of the addition, under nitrogen protection, the mixture was stirred at 100° C. for 1 hour, cooled to room temperature, quenched by adding water (100 mL) and extracted with ethyl acetate (80 mL*2); the organic phase was washed with saturated brine (100 mi,*3) and concentrated: the crude product was separated and purified through a flash column chromatography (EA/PE=1/1) to give compound 3-d (0.5 g, 44% yield for two steps) as a white solid. LC-MS (ESI): m/z=566.3 [M+1]⁺.

### Synthesis of Compound 3-c

Compound 3-d (0.5 g, 0.9 mmol) was dissolved in ethyl acetate (20 mL) and MCPBA (0.46 g, 2.7 mmol) was added at room temperature. The mixture was stirred at room temperature for 1 hour and then quenched by adding saturated sodium bicarbonate solution (50 mL), extracted with ethyl acetate (50 mL*2), filtered and concentrated: the crude product was separated and purified through a flash column chromatography (DCM/MeOH=9/1) to give solid compound 3-c (0.38 g, 72%).

### Synthesis of Compound 3-b

In an ice-water bath, to a solution of compound 3-c (0.38 g, 0.63 mmol) in toluene (15 mL) were successively added N-methyl-L-prolinol (0.1 mL, 0.9 mmol) and t-BuONa (0.1 g, 0.9 mmol). After completion of the addition, under nitrogen atmosphere, the mixture was stirred in an ice-water bath for 0.5 hours and then quenched by adding water (10 mL) and extracted with ethyl acetate (30 mL*2); the organic phase was concentrated: the crude product was separated and purified through a flash column chromatography (MeOH/DCM=1/9) to give compound 3-b (0.3 g, 75%) as a white solid. LC-MS (ESI): m/z=633.3 [M+1.]⁺.

### Synthesis of Compound 3-a

A solution of compound 3-b (0.13 g, 0.2 mmol) in methanolic ammonia (7 M, 50 mL) was cooled to −78° C., replaced twice with nitrogen, and then was added 10% Pd—C (55 mg) and replaced three times with hydrogen. The reaction mixture was warmed to room temperature and stirred under hydrogen for 2 hours. The reaction mixture was filtered and concentrated to give compound 3-a (0.1 g, 100%). LC-MS (ESI): m/z=499.3 [M+1]⁺.

### Synthesis of Compound 3

At room temperature, compound 3-a (0.1 g, 0.2 mmol) was dissolved in DCM (10 mL), and DIPEA (75 μL, 0.45 mmol) and acryloyl chloride (25 μL, 0.23 mmol) were successively added. Under nitrogen atmosphere, the mixture was stirred at room temperature overnight, quenched by adding water (10 mL) and extracted with DCM (50 mL*3); the organic phase was concentrated: the crude product was separated and purified through a flash column chromatography (MeOH/DCM=119) to give compound 3 (8 mg, 7%) as a white solid. LC-MS (ESI): m/z=553.3 [M+1]⁺: ¹H NMR (500 MHz, CD₃OD): δ 8.07 (d, 1H, J=7.5 Hz), 7.82 (d, 1H, J=9.5 Hz), 7.78 (d, 1H, J=8.5 Hz), 7.50-7.59 (m, 1H), 7.36-7.48 (m, 3H), 6.62-6.86 (m, 1H), 6.19 (d, 1H, J=17.0 Hz), 5.96-6.08 (m, 2H), 5.73 (d, 1H, J=9.5 Hz), 5.53 (dd, 1H, J=4.5 Hz, J=11.0 Hz), 5.46 (dd, 111, J=2.5 Hz, J=9.0 Hz), 4.93-5.07 (m, 2H), 4.67-4.76 (m, 1H), 4.43-4.52 (m, 2H), 4.35-4.42 (m, 1H), 3.91-4.08 (m, 1H), 3.28-3.64 (m, 1H), 3.26-3.36 (m, 2H), 3.09-3.18 (m, 2H), 2.90-3.02 (m, 2H), 2.78-2.86 (m, 1H), 2.69 (d, 3H, J=12.5 Hz), 2.06-2.18 (m, 1H), 1.86-1.93 (m, 2H), 1.73-1.77 (m, 1H).

### Synthesis of Compounds 3-1 and 3-2

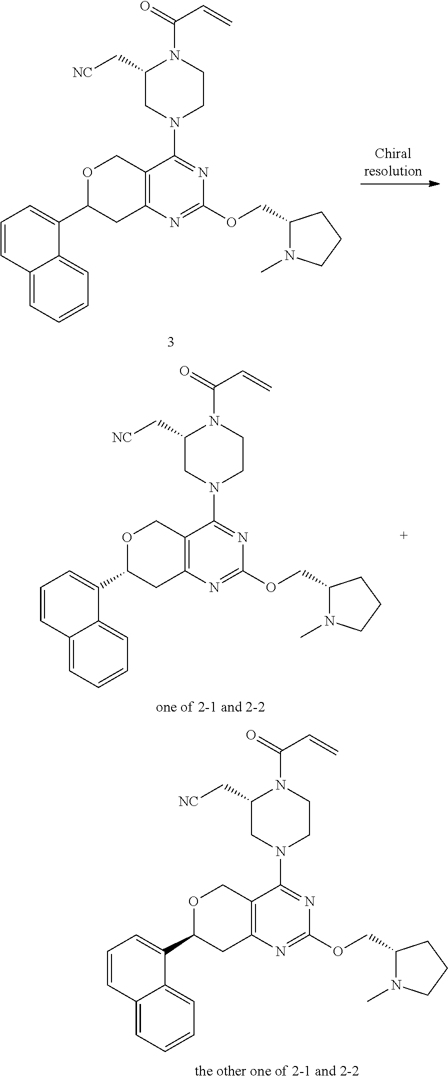 [SMILES: C=CC(=O)N1CCN(c2nc(OC[C@@H]3CCCN3C)nc3c2COC(c2cccc4ccccc24)C3)C[C@@H]1CC#N ; C=CC(=O)N1CCN(c2nc(OC[C@@H]3CCCN3C)nc3c2CO[C@@H](c2cccc4ccccc24)C3)C[C@@H]1CC#N ; C=CC(=O)N1CCN(c2nc(OC[C@@H]3CCCN3C)nc3c2CO[C@H](c2cccc4ccccc24)C3)C[C@@H]1CC#N]

According to the method for synthesizing compound 3, compound 3 (170 mg) was synthesized and purified by chiral resolution to give compound 3-1 (40 mg, 24%) as a white solid and compound 3-2 (20 mg, 12%) as a white solid.

| Chiral analysis conditions | Chiral preparation conditions |
|---|---|
| equipment: SFC Method Station | instrument: SFC-150 (Thar, |
| (Thar, Waters) | Waters) |
| chromatographic column: AD-H | chromatographic column: AD |
| 4.6 * 100 mm, 5 μm (Daicel) | 20 * 250 mm. 10 μm (Daicel) |
| column temperature: 40° C. | column temperature: 35° C. |
| mobile phase: CO₂/ETOH (0.5% | mobile phase: CO₂/ETOH (0.5% |
| TEA) = 55/45 | TEA) = 40/60 |
| flow rate: 4.0 ml/min | flow rate: 120 g/min |
| wavelength: 254 nm | back pressure: 100 bar |
| back pressure: 120 bar | detection wavelength: 214 nm |
| | cycling time: 5.0 min |
| | sample solution: 170 mg dissolved |
| | in 20 ml of methanol |
| 3-1: retention time: 0.97 min; | |
| d.e. % = 93.5%; | |
| 3-2: retention time: 2.40 min; | |
| d.e. % = 99.4%. | |

3-1: LC-MS (ESI): m/z=553.0 [M+1]⁺: ¹H NMR (400 MHz, MeOD): δ 8.06 (d, 1H, J=7.6 Hz), 7.82 (d, 1H, J=7.6 Hz), 7.76 (d, 1H, J=8.4 Hz), 7.57 (d, 1H, J=6.8 Hz), 7.38-7.44 (m, 3H), 6.62-6.86 (m, 1H), 6.19 (d, 1H, J=16.0 Hz), 5.73 (d, 1H, J=10.4 Hz), 5.51 (dd, 1H, J=10.4, 4.0 Hz), 5.02 (d, 1H, J=13.6 Hz), 4.64-4.98 (m, 1H), 4.37-4.59 (m, 1H), 4.19-4.31 (m, 2H), 3.93-3.06 (m, 1H), 3.81-3.91 (m, 1H), 3.57-3.74 (m, 1H), 3.28-3.50 (m, 1H), 2.89-3.16 (m, 5H), 2.76-2.86 (m, 1H), 2.66-2.75 (m, 1H), 2.42 (s, 3H), 2.24-2.33 (m, 1H), 1.95-2.05 (m, 1H), 1.68-1.78 (m, 2H), 1.55-1.67 (m, 1H), 1.21-1.28 (m, 1H).

3-2: LC-MS (ESI): z=553.0 [M+1]⁺: ¹H NMR (400 MHz, MeOD): δ 8.07 (d, 1H, J=8.0 Hz), 7.82 (d, 1H, J=7.2 Hz), 7.76 (d, 1H, J=8.0 Hz), 7.54 (d, 1H, J=7.2 Hz), 7.37-7.44 (m, 3H), 6.33-6.85 (m, 1H), 6.19 (d, 1H, J=16.4 Hz), 5.74 (d, 1H, J=10.4 Hz), 5.50 (dd, 1H, J=10.4, 4.0 Hz), 4.97 (d, 1H, J=14.0 Hz), 4.61-4.75 (m, 1H), 4.37-4.57 (m, 1H), 4.21-4.34 (m, 2H), 4.14 (d, 1H, J=13.6 Hz), 3.92-4.06 (m, 1H), 3.70-3.81 (m, 1H), 3.27-3.42 (m, 1H), 2.89-3.17 (m, 611), 2.69-2.80 (m, 1H), 2.46 (s, 3H), 2.30-2.39 (m, 1H), 1.96-2.12 (m, 1H), 1.70-1.79 (m, 2H), 1.60-1.69 (m, 1H), 1.20-1.27 (m, 1H).

### Example 4 Synthetic Route of Compound 4

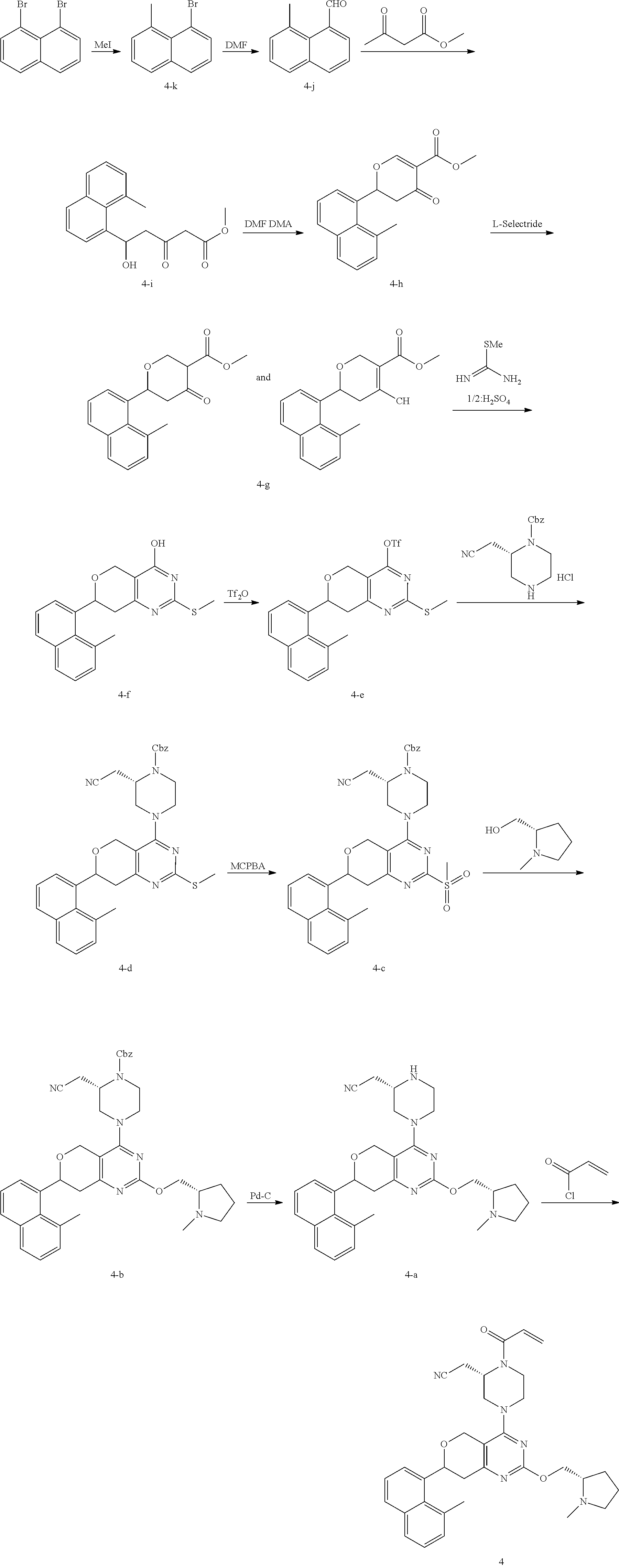

### Synthesis of Compound 4-k

A solution of compound 1,8-dibromonaphthalene (5 g, 17.48 mmol) in THF (40 mL) was cooled to −78° C., and under nitrogen protection, n-BuLi (2.5 M, 7.5 mL, 18.75 mmol) was added dropwise. After completion of the addition, the mixture was stirred at −78° C. for 20 minutes, and then iodomethane (2.2 mL, 35.2 mmol) was added dropwise at −78° C. After completion of the addition, the reaction mixture was warmed to room temperature and stirred for 1 how. The reaction mixture was then poured into 50 mL of saturated brine and extracted with ethyl acetate (100 mL*2); the organic phase was dried over anhydrous sodium sulfate, filtered and concentrated to give a crude product. The crude product was separated and purified through a flash column chromatography (PE) to give compound 4-k (3.28 g, 85% yield) as a white solid. ¹H NMR (500 MHz, CDCl₃): δ 7.85 (d, 1H, J=7.0 Hz), 7.80 (d, 1H, J=7.5 Hz), 7.75-7.71 (m, 1H), 7.40-7.33 (m, 2H), 7.23 (t, 1H, J=8.0 Hz), 3.15 (s, 3H).

### Synthesis of Compound 4-j

A solution of compound 4-k (3.28 g, 14.84 mmol) in THF (110 mL) was cooled to −78° C.: wider nitrogen protection, n-BuLi (2.5 M, 12 mL, 30 mmol) was added dropwise. After completion of the dropwise addition, the mixture was stirred at −78° C. for 10 minutes, and then DMF (5.8 mL, 74.55 mmol) was added dropwise at −78° C. After completion of the addition, the reaction mixture was stirred at −78° C. for 30 minutes and then warmed to room temperature and stirred for 2 hours: the reaction was quenched with 20 mL of saturated ammonium chloride solutions, and then added to 100 mL of saturated sodium bicarbonate solution and extracted with ethyl acetate (100 mL); the organic phase was washed with saturated brine (100 mL*2), dried over anhydrous sodium sulfate, filtered and concentrated to give a crude product. The crude product was separated and purified through a flash column chromatography (EA/PE=1/10) to give compound 4-j (1.5 g, 60% yield) as a white solid. LC-MS (ESI): m/z=171.2 [M+H]⁺: ¹H NMR (400 MHz, CDCl₃): δ 10.85 (s, 1H), 7.97 (dd, 1H, J₁=1.2 Hz, J₂=8 Hz), 7.89 (dd, 1H, J₁=1.6 Hz, J₂=7.2 Hz), 7.74-7.69 (m, 1H), 7.46 (t, 1H, J=8 Hz), 7.42-7.36 (m, 2H), 2.75 (s, 3H).

### Synthesis of Compound 4-i

At room temperature. NaH (60%, 423 mg, 10.58 mmol) was added to 10 mL, of THF. Under nitrogen atmosphere, methyl acetoacetate (950 μL, 8.82 mmol) was added at room temperature. Under nitrogen atmosphere, the mixture was stirred at room temperature for 30 minutes, and then n-BuLi (2.5 M 4.2 mL, 10.5 mmol) was added dropwise at −15° C., to −10° C. After completion of the addition, the mixture was kept at this temperature for 30 minutes, and then a solution of compound 4-j (500 mg, 2.94 mmol) in THF (10 mL) was added dropwise. After completion of the addition, the mixture was stirred at low temperature (−10° C., to 0° C.) for 2 hours, and then the reaction was quenched with a saturated ammonium chloride solution (100 mL) and extracted with ethyl acetate (80 mL*2). The organic phase was washed with saturated brine (100 mL), dried over anhydrous sodium sulfate, filtered and concentrated to give a crude product: the crude product was separated and purified through a flash column chromatography (EA/DCM=1/10) to give compound 4-i (806 mg, 96% yield) as a white solid. LC-MS (ESI): m/z=309.1 [M+Na]⁺: ¹H NMR (400 MHz, CDCl₃): δ 7.92 (d, 1H, J=7.2 Hz), 7.81 (dd, 1H, J₁=1.2 Hz, J₂=8.4 Hz), 7.77-7.72 (m, 1H), 7.49 (t, 1H, J=7.2 Hz), 7.39-7.33 (m, 2H), 6.47 (d, 1H, J=9.6 Hz), 3.76 (s, 3H), 3.55 (s, 2H), 3.10-2.92 (m, 3H), 2.89 (s, 3H).

### Synthesis of Compound 4-h

Compound 4-i (800 mg, 2.79 mmol) was dissolved in DCM (30 mL) at room temperature; under nitrogen atmosphere, DMF-DMA (412 μL, 3.08 mmol) was added at room temperature. At room temperature, the reaction mixture was stirred for 45 minutes, and then BF₃-Et₂O (390 μL, 3.08 mmol) was added. After completion of the addition, the mixture was stirred at room temperature for 1 hour and then diluted with 200 mL of ethyl acetate. The organic phase was washed successively with a saturated NaHCO₃ solution (200 mL) and saturated brine (200 mL), dried over anhydrous sodium sulfate, filtered and concentrated to give crude product compound 4-h (870 mg). The crude product was used directly in the next reaction without purification. LC-MS (ESI): m/z=297.1 [M+H]⁺.

### Synthesis of Compound 4-g

At room temperature, compound 4-h (770 mg, 2.59 mmol) was dissolved in TIE′ (60 mL); at −78° C., under nitrogen atmosphere, a solution of tri-sec-butyl lithium borohydride in tetrahydrofuran (1 M, 2.6 mL, 2.6 mmol) was added dropwise. After completion of the addition, the mixture was stirred at −78° C. for 1 hour; the reaction was then quenched by adding a saturated ammonium chloride solution (50 mL) and extracted with ethyl acetate (100 mL*2); the organic phase was washed with saturated brine (100 mL*2), dried over anhydrous sodium sulfate, filtered and concentrated to give a crude product: the crude product was separated and purified through a flash column chromatography (PE/EA=4/1) to give compound 4-g (670 mg, 86% yield) as a yellow oil. LC-MS (ESI): m/z=299.2 [M+1]⁺.

### Synthesis of Compound 4-f

At room temperature, compound 4-g (670 mg, 2.25 mmol) was dissolved in methanol (50 mL); at 0° C. under nitrogen atmosphere, sodium methylate (608 mg, 11.25 mmol) and compound 2-methyl-2-thiourea sulfate (563 mg, 2.02 mmol) were successively added. After completion of the addition, the mixture was warmed to room temperature and stirred for 20 hours. The pH of the reaction mixture was adjusted to 5 with 1 M dilute hydrochloric acid: the solid was precipitated out and filtered: the filter cake was washed with water (5 mL*2) to collect a solid, which was dried in vacuum to give crude product 4-f (459 mg, 60% yield) as a white solid. LC-MS (ESI): 339.1 [M+1]⁺.

### Synthesis of Compound 4-e

At room temperature, compound 4-f (459 mg, 1.36 mmol) was dissolved in DCM (18 mL): in an ice-water bath, under nitrogen atmosphere. DIPEA (673 μL, 4.08 mmol) and trifluoromethanesulfonic anhydride (343 μL, 2.04 mmol) were successively added. After completion of the addition, the reaction mixture was stirred in an ice-water bath for 2 hours and then quenched with a saturated sodium bicarbonate solution (50 mL) and extracted with DCM (50 mL*2); the organic phase was dried over anhydrous sodium sulfate, filtered and concentrated to give a crude product: the crude product was separated and purified through a flash column chromatography (EA/PE=1/10) to give compound 4-e (432 mg, 68% yield) as a white solid. LC-MS (ESI): m/z=471.1 [M+1]⁺.

### Synthesis of Compound 4-d

At room temperature, compound 4-e (430 mg, 0.91 mmol) was dissolved in DMF (10 mL), and then DIPEA (453 μL, 2.75 mmol) and benzyl (S)-2-cyanomethylpiperazine-1-carboxylate hydrochloride (324 me, 1.1 mmol) were successively added. After completion of the addition, under nitrogen protection, the mixture was stirred at 100° C. for 1 hour and then cooled to room temperature: the reaction was quenched with saturated brine (100 mL) and extracted with ethyl acetate (80 mL*2). The organic phase was washed with saturated brine (100 mL*3) and then dried over anhydrous sodium sulfate, filtered and concentrated to give a crude product: the crude product was separated and purified through a flash column chromatography (EA/PE=1/1) to give compound 4-d (470 mg, 68% yield) as a white solid. LC-MS (ESI): m/z=580.3 [M+1]⁺.

### Synthesis of Compound 4-c

At room temperature, compound 4-d (200 mg, 0.34 mmol) was dissolved in ethyl acetate (20 mL), and MCPBA (175 mg, 0.86 mmol) was added. After completion of the addition, the mixture was stirred at room temperature for 1 hour and then quenched with a saturated sodium bicarbonate solution (50 mL) and extracted with ethyl acetate (50 mL*2); the organic phase was dried over anhydrous sodium sulfate, filtered and concentrated to give a crude product: the crude product was separated and purified through a flash column chromatography (EA/PE=114) to give compound 4-c (210 mg, 99% yield) as a white solid. LC-MS (ESI): m/z=612.3 [MH-1]′.

### Synthesis of Compound 4-b

At room temperature, compound 4-c (100 mg, 0.16 mmol) was dissolved in toluene (5 mL), and then the reaction mixture was cooled to 0° C.: N-methyl-L-prolinol (34 μL, 0.29 mmol) and t-BuONa (32 mg, 0.33 mmol) were successively added. After completion of the addition, under nitrogen atmosphere, the reaction mixture was stirred in an ice-water bath for 0.5 hours and then quenched with water (10 mL) and extracted with ethyl acetate (30 mL*2).

The organic phase was dried over anhydrous sodium sulfate, filtered and concentrated to give a crude product: the crude product was separated and purified through a flash column chromatography (MeOH/DCM=1120) to give compound 4-b (97 mg, 92% yield) as a white solid. LC-MS (ESI): m/z=647.4 [M+1]⁻.

### Synthesis of Compound 4-a

At room temperature, compound 4-b (90 me, 0.14 mmol) was dissolved in methanolic ammonia (7 M, 50 mL); the reaction mixture was cooled to −78° C. replaced twice with nitrogen, and then was added 10% Pd—C (75 mg) and replaced three times with hydrogen; the reaction mixture was warmed to room temperature and stirred under hydrogen for 2 hours. The reaction mixture was filtered and concentrated to give compound 4-a (77 mg, 99% yield) as a white solid. LC-MS (ESI): m/z=513.3 [M+1]⁺.

### Synthesis of Compound 4

At room temperature, compound 4-a (77 mg, 0.15 mmol) was dissolved in DCM (10 mL), and DIPEA (75 μL, 0.45 mmol) and acryloyl chloride (25 μL, 0.23 mmol) were successively added. After completion of the addition, under nitrogen atmosphere, the reaction mixture was stirred at room temperature for 20 hours and then quenched with water (10 mL) and extracted with DCM (50 mL*2). The organic phase was dried over anhydrous sodium sulfate, filtered and concentrated to give a crude product: the crude product was separated and purified through a flash column chromatography (MeOH/DCM=1/10) to give compound 4 (56 mg, 66% yield) as a white solid. LC-MS (ESI): m/z=567.3 [M+1]⁺: ¹H NMR (400 MHz, CDCl₃): δ 7.78-7.65 (m, 3H), 7.45-7.36 (m, 1H), 7.31-7.26 (m, 2H), 6.55-6.44 (m, 1H), 6.35-6.27 (m, 1H), 5.95-5.88 (m, 1H), 5.75 (d, 111, J=10.4 Hz), 4.94-4.84 (m, 1H), 4.81-4.59 (m, 2H), 4.52-4.37 (m, 1H), 4.22-4.11 (m, 1H), 3.96-3.76 (m, 2H), 3.68-3.58 (m, 1H), 3.48-3.37 (m, 1H), 3.24-2.88 (m, 5H), 2.85 (d, 3H, J=11.6 Hz), 2.78-2.58 (m, 2H), 2.50 (s, 3H), 2.40-2.27 (m, 1H), 2.08-1.68 (m, 5H).

### Synthesis of Compounds 4-1 and 4-2

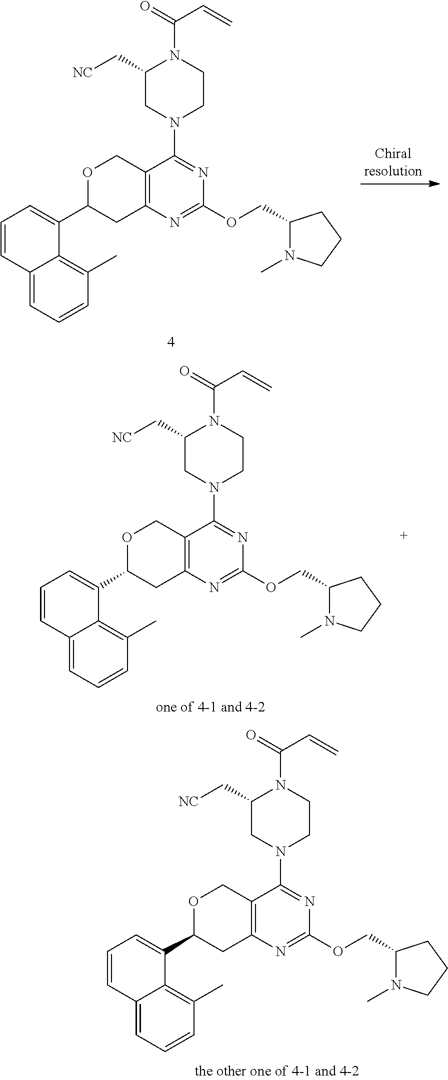 [SMILES: C=CC(=O)N1CCN(c2nc(OC[C@@H]3CCCN3C)nc3c2COC(c2cccc4cccc(C)c24)C3)C[C@@H]1CC#N ; C=CC(=O)N1CCN(c2nc(OC[C@@H]3CCCN3C)nc3c2CO[C@@H](c2cccc4cccc(C)c24)C3)C[C@@H]1CC#N ; C=CC(=O)N1CCN(c2nc(OC[C@@H]3CCCN3C)nc3c2CO[C@H](c2cccc4cccc(C)c24)C3)C[C@@H]1CC#N]

According to the method for synthesizing compound 4, compound 4 (140 mg) was synthesized and purified by chiral resolution to give compound 4-1 (30 mg, 21% yield) as a white solid and compound 4-2 (40 mg, 29% yield) as a white solid.

| Chiral analysis conditions | Chiral preparation conditions |
|---|---|
| equipment: SFC Method Station | equipment: SFC-150 (Thar, |
| (Thar, Waters) | Waters) |
| chromatographic column: OJ-H | chromatographic column: OJ |
| 4.6 * 100 mm, 5 μm (Daicel) | 20 * 250 mm. 10 μm (Daicel) |
| column temperature: 40° C. | column temperature: 35° C. |
| mobile phase: CO₂/Methanol (0.1% | mobile phase: CO₂/Methanol |
| TEA) = 60/40 | (0.1% TEA) = 45/55 |
| flow rate: 4.0 ml/min | flow rate: 120 g/min |
| wavelength: 254 nm | column pressure: 100 bar |
| back pressure: 120 bar | wavelength: 214 nm |
| | cycling time: 6 min |
| 4-1: retention time: 0.97 min, | |
| d.e. % = 100.0%; | |
| 4-2: retention time: 1.94 min, | |
| d.e. % = 98.1%. | |

4-1: LC-MS (ESI): m/z=567.3 [M+H]⁺: ¹H NMR (400 MHz, CDCl₃) δ 7.86 (d, J=8 Hz, 1H), 7.80-7.74 (m, 2H), 7.47 (t. J=8 Hz, 1H), 7.41-7.34 (m, 2H), 6.65-6.50 (m, 1H), 6.40 (d, J=16.4 Hz, 1H), 6.01 (dd, J=8.8, 3.6 Hz, 1H), 5.84 (d, J=10.4 Hz, 1H), 5.17-4.98 (m, 1H), 4.87 (d, J=13.6 Hz, 1H), 4.71 (d, J=13.2 Hz, 1H), 4.43 (dd, J=10.8, 4.8 Hz, 1H), 4.21 (dd, J=10, 6.4 Hz, 1H), 4.03-3.38 (m, 3H), 3.34-3.05 (m, 5H), 3.03-2.97 (m, 1H), 2.95 (s, 3H), 2.84-2.70 (m, 2H), 2.52 (s, 3H), 2.34-2.29 (m, 1H), 2.14-2.00 (m, 2H), 1.92-1.82 (m, 2H), 1.39-1.32 (m, 1H).

4-2: LC-MS (ESI): m/z=567.3 [M+H]⁺: ¹H NMR (400 MHz, CDCl₃) δ 7.85 (d, J=7.6 Hz, 2H), 7.76 (d, J=6.4 Hz, 1H), 7.51 (t. J=7.6 Hz, 111), 7.39-7.35 (m, 2H), 6.68-6.52 (m, 1H), 6.40 (d, J=16.4 Hz, 1H), 6.01 (dd, J=10.4, 2.8 Hz, 1H), 5.84 (d, J=10 Hz, 1H), 5.11-4.92 (m, 2H), 4.80 (d, J=13.6 Hz, 1H), 4.49-4.38 (m, 1H), 4.23-4.15 (m, 1H), 4.04-3.68 (m, 3H), 3.58-3.45 (m, 1H), 3.36-3.00 (m, 5H), 2.92 (s, 3H), 2.78-2.69 (m, 2H), 2.51 (s, 3H), 2.38-2.28 (m, 1H), 2.16-2.01 (m, 2H), 1.91-1.81 (m, 2H), 1.38-1.33 (m, 1H).

### Example 5 Synthetic Route of Compound 5

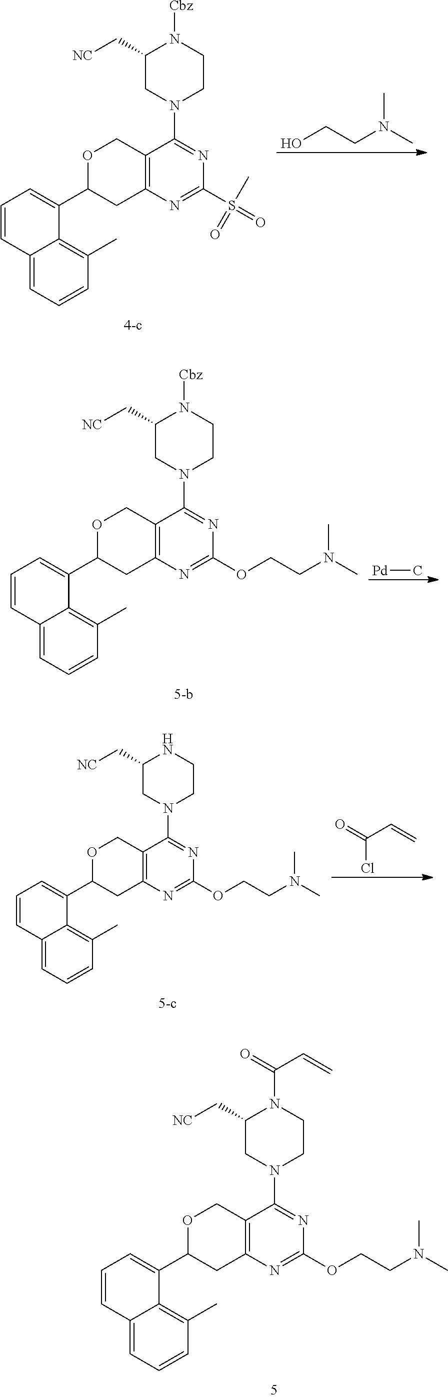

### Synthesis of Compound 5-b

Compound 4-c (100 mg, 0.164 mmol) was dissolved in toluene (5 mL): under ice-water bath cooling, 2-dimethylaminoethanol (29 μL, 0.29 mmol) and t-BuONa (32 mg, 0.33 mmol) were successively added. Under nitrogen atmosphere, the mixture was stirred in an ice-water bath for 0.5 hours and then quenched with water (10 mL) and extracted with ethyl acetate (30 mL*2). The organic phase was dried over anhydrous sodium sulfate, filtered and concentrated to give a crude product: the crude product was separated and purified through a flash column chromatography (MeOH/DCM=1/10) to give compound 5-b (100 mg, 98% yield) as a white solid. LC-MS (ESI): m/z=621.4 [M+H]⁺.

### Synthesis of Compound 5-a

Compound 5-b (100 mg, 0.161 mmol) was dissolved in methanolic ammonia (7 M, 50 mL), cooled to −78° C. replaced twice with nitrogen, and then added Pd—C (75 mg). After replaced three times with hydrogen, the reaction mixture was warmed to room temperature and stirred under hydrogen for 2 hours. The reaction mixture was filtered and concentrated to give compound 5-a (80 mg, 97% yield) as a white solid. LC-MS (ESI): m/z=487.3 [M+1]⁺.

### Synthesis of Compound 5

At room temperature, compound 5-a (80 mg, 0.165 mmol) was dissolved in DCM (10 mL), and DIPEA (82 μL, 0.495 mmol) and acryloyl chloride (30 μL, 0.25 mmol) were successively added. Under nitrogen atmosphere, the mixture was stirred at room temperature for 20 hours and then quenched with water (10 mL) and extracted with DCM (50*2); the organic phase was dried over anhydrous sodium sulfate, filtered and concentrated to give a crude product: the crude product was separated and purified through a flash column chromatography (MeOH/DCM=10%) to give compound 5 (55 mg, 62% yield) as a white solid. LC-MS (ESI): m/z=541.3 [M+1]⁻: ¹H NMR (400 MHz, CDCl₃); δ 7.88-7.65 (m, 3H), 7.56-7.40 (m, 1H), 7.38-7.12 (m, 2H), 6.68-6.25 (m, 2H), 6.05-5.66 (m, 2H), 5.44-4.54 (m, 3H), 4.44 (s, 2H), 4.05-3.79 (m, 2H), 3.77-3.33 (m, 3H), 3.29-2.99 (m, 4H), 2.91 (d, 3H, J=12.4 Hz), 2.83-2.56 (m, 3H), 2.36 (s, 6H).

### Example 6 Synthetic Route of Compound 6

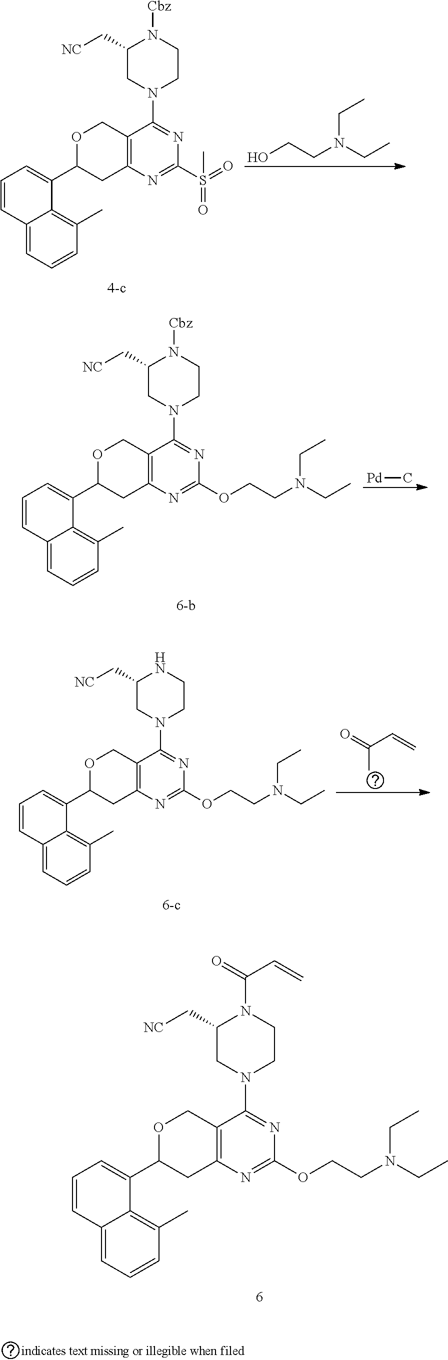

### Synthesis of Compound 6-b

Compound 4-c (62 mg, 0.10 mmol) was dissolved in toluene (5 mL); under ice-water bath cooling, 2-diethylaminoethanol (24 μL, 0.18 mmol) and t-BuONa (20 mg, 0.20 mmol) were successively added. Under nitrogen atmosphere, the mixture was stirred in an ice-water bath for 0.5 hours and then quenched with water (10 mL) and extracted with ethyl acetate (25 mL*2). The organic phase was dried over anhydrous sodium sulfate, filtered and concentrated to give a crude product: the crude product was separated and purified through a flash column chromatography (MeOH/DCM=1/10) to give compound 6-b (65 mg, 99% yield) as a white solid. LC-MS (ESI): m/z=649.4 [M+1]⁺.

### Synthesis of Compound 6-a

Compound 6-b (65 mg, 0.1 mmol) was dissolved in methanolic ammonia (7 M, 50 mL), cooled to −78° C., replaced twice with nitrogen, and then added Pd/C (30 mg). After replaced three times with hydrogen, the reaction mixture was warmed to room temperature and stirred under hydrogen for 1 hour. The reaction mixture was filtered and concentrated to give compound 6-a (51 mg, 99% yield) as a white solid. LC-MS (ESI): m/z=515.3 [M+1]⁺.

### Synthesis of Compound 6

At room temperature, compound 6-a (51 mg, 0.10 mmol) was dissolved in DCM (10 mL), and DIPEA (82 μL, 0.50 mmol) and acryloyl chloride (13.6 mg, 0.15 mmol) were successively added. After completion of the addition, under nitrogen atmosphere, the mixture was stirred at room temperature for 20 hours and then quenched with water (10 mL) and extracted with DCM (50 mL*2). The organic phase was dried over anhydrous sodium sulfate, filtered and concentrated to give a crude product: the crude product was separated and purified through a flash column chromatography (MeOH/DCM=1/10) to give compound 6 (50 mg, 68% yield) as a white solid. LC-MS (ESI): m/z=569.3 [M+1]⁺; NMR (400 MHz, CDCl₃) δ 7.85-7.71 (m, 3H), 7.52-7.43 (iii, 1H), 7.39-7.31 (m, 2H), 6.67-6.47 (m, 1H), 6.42-6.24 (m, 1H), 6.17-5.95 (m, 1H), 5.85-5.66 (m, 1H), 5.08-4.92 (m, 1H), 4.89-4.66 (m, 4H), 4.08-3.85 (m, 2H), 3.80-3.53 (m, 2H), 3.32-3.15 (m, 4H), 3.12-2.97 (m, 6H), 2.92 (d, 3H, J=12.8 Hz), 2.85-2.62 (m, 2H), 1.34-1.27 (m, 6H).

### Synthesis of Compounds 6-1 and 6-2

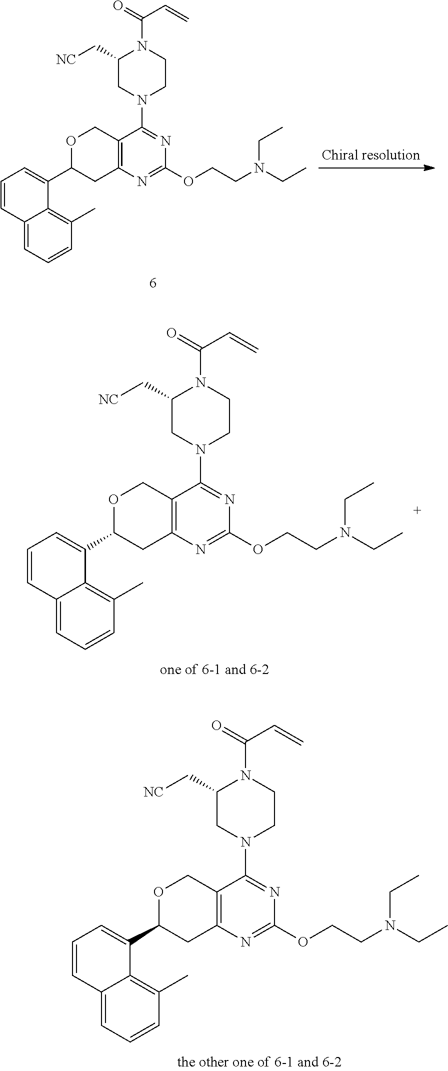 [SMILES: C=CC(=O)N1CCN(c2nc(OCCN(CC)CC)nc3c2COC(c2cccc4cccc(C)c24)C3)C[C@@H]1CC#N ; C=CC(=O)N1CCN(c2nc(OCCN(CC)CC)nc3c2CO[C@@H](c2cccc4cccc(C)c24)C3)C[C@@H]1CC#N ; C=CC(=O)N1CCN(c2nc(OCCN(CC)CC)nc3c2CO[C@H](c2cccc4cccc(C)c24)C3)C[C@@H]1CC#N]

Compound 6 (40 mg, 0.07 mmol) was subjected to chiral resolution to give compound 6-1 (12 mg, 30% yield) as a white solid and compound 6-2 (11 me, 28% yield) as a white solid.

| Chiral analysis conditions | Chiral preparation conditions |
|---|---|
| instrument: SFC Method Station | instrument: SFC-80 (Thar, |
| (Thar, Waters) | Waters) |
| chromatographic column: | chromatographic column: |
| CHIRALCEL OJ-H 4.6 * 100 | CHIRALCEL OJ-H 20 * 250 |
| mm, 5 μm (Daicel) | mm. 5 μm (Daicel) |
| column temperature: 40° C. | column temperature: 35° C. |
| mobile phase: CO₂/MeOH (0.1% | mobile phase: CO₂/MeOH (0.1% |
| TEA) = 65/35 | TEA) = 65/35 |
| back pressure: 120 bar | flow rate: 80 g/min |
| flow rate: 1.0 ml/min | back pressure: 100 bar |
| | detection wavelength: 214 nm |
| 6-1: retention time: 1.22, | |
| d.e. % = 100%; | |
| 6-2: retention time: 2.67, | |
| d.e. % = 96.7%. | |

6-1: LC-MS (ESI): m/z=569.3 [M+1]⁺: ¹H NMR (500 MHz, CDCl₃): δ 7.83 (d, 1H, J=8 Hz), 7.75 (t, 2H, J=7 Hz), 7.45 (t, 1H, J=8 Hz), 7.39-7.31 (m, 2H), 6.63-6.49 (m, 1H), 6.38 (d, 1H, J=17 Hz), 6.00 (dd, 1H, J₁=4 Hz, J₂=9.5 Hz), 5.81 (d, 1H, J=11 Hz), 5.17-4.92 (m, 1H), 4.86 (d, 1H, J=14 Hz), 4.71 (d, 1H, J=14 Hz), 4.39 (t, 2H, J=6.5 Hz), 4.11-3.78 (m, 2H), 3.70 (d, 1H, J=12 Hz), 3.53-3.33 (m, 1H), 3.25 (dt, 2H, J₁=3.5 Hz. J₂=18.5 Hz), 3.17-3.03 (m, 2H), 3.02-2.95 (m, 1H), 2.94 (s, 3H), 2.87 (t, 2H, J=6.5 Hz), 2.83-2.71 (m, 1H), 2.63 (q, 4H, J=6.5 Hz), 1.06 (t, 6H, J=7 Hz).

6-2: LC-MS (ESI): m/z=569.3 [M+H]⁺: ¹H NMR (500 MHz, CDCl₃): δ 7.87-7.79 (m, 2H), 7.75 (d, 1H, J=8 Hz), 7.49 (t, 1H, J=7.5 Hz), 7.39-7.30 (m, 2H), 6.65-6.48 (m, 1H), 6.38 (d, 1H, J=16.5 Hz), 5.99 (dd, 1H, J₂=3.5 Hz, J=10.5 Hz), 5.82 (d, 1H, J=10.5 Hz), 4.99 (d, 2H, J=13.5 Hz), 4.78 (d, 1H, J=14 Hz), 4.73-4.45 (m, 1H), 4.38 (t, 2H, J=6.5 Hz), 3.96 (d, 1H, J=14 Hz), 3.92-3.78 (m, 1H), 3.75-3.57 (m, 1H), 3.55-3.36 (m, 1H), 3.25 (dd, 1H, J₁=3 Hz, J₂=18.5 Hz), 3.15-2.96 (m, 2H), 2.91 (s, 3H), 2.86 (t, 2H, J=6.5 Hz), 2.80-2.66 (m, 2H), 2.62 (q, 4H, J=7 Hz), 1.05 (t, 6H, J=7 Hz).

### Example 7 Synthetic Route of Compound 7

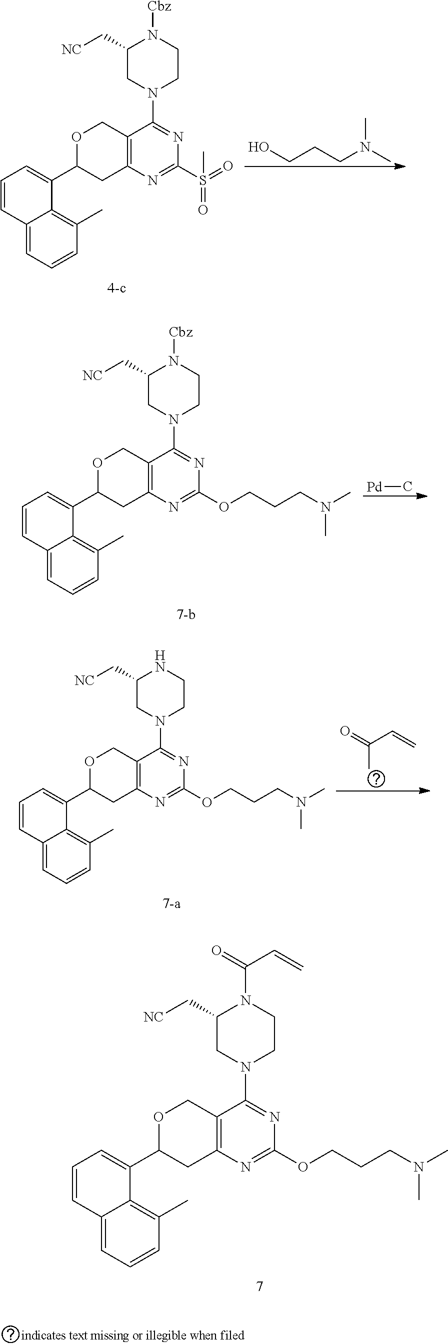

### Synthesis of Compound 7-b

Compound 4-c (62 mg, 0.10 mmol) was dissolved in toluene (5 mL): wider ice-water bath cooling, 3-dimethylamino-1-propanol (21 μL, 0.18 mmol) and t-BuONa (20 mg, 0.20 mmol) were successively added. Under nitrogen atmosphere, the mixture was stirred in an ice-water bath for 0.5 hours, warmed to room temperature, stirred for 20 hours and then slowly heated to 100° C., and stirred for about 2 hours. The reaction mixture was cooled to room temperature, quenched with water (50 mL) and extracted with ethyl acetate (50 mL*2). The organic phase was dried over anhydrous sodium sulfate, filtered and concentrated to give a crude product: the crude product was separated and purified through a flash column chromatography (MeOH/DCM=1/10) to give compound 7-b (38 mg, 60% yield) as a white solid. LC-MS (ESI): m/z=635.4 [M+H]⁺.

### Synthesis of Compound 7-a

Compound 7-b (38 mg, 0.06 mmol) was dissolved in methanolic ammonia (7 M, 20 mL), cooled to −78° C. replaced twice with nitrogen, and then added Pd/C (20 mg). After replaced three times with hydrogen, the reaction mixture was warmed to room temperature and stirred under hydrogen for 1 hour: the reaction mixture was filtered and concentrated to give compound 7-a (35 mg, 99% yield) as a white solid. LC-MS (ESI): m/z=501.3 [M+1]⁺.

### Synthesis of Compound 7

Compound 7-a (35 mg, 0.07 mmol) was dissolved in DCM (10 mL), and DIPEA (58 μL, 0.35 mmol) and acryloyl chloride (10 mg, 0.11 mmol) were successively added. Under nitrogen atmosphere, the mixture was stirred at room temperature for 20 hours and then quenched with a saturated sodium bicarbonate solution (20 mL) and extracted with DCM (30 mL*2). The organic phase was dried over anhydrous sodium sulfate, filtered and concentrated to give a crude product: the crude product was separated and purified through a flash column chromatography (MeOH/DCM=1/10) to give compound 7 (15 mg, 38% yield) as a white solid. LC-MS (ESI): m/z=555.3 [M+1]⁺: ¹H NMR (400 MHz, CDCl₃); δ 7.87-7.59 (m, 3H), 7.53-7.41 (m, 1H), 7.41-7.30 (m, 2H), 6.63-6.48 (m, 1H), 6.38 (d, 1H, J=15.6 Hz), 6.19-6.05 (m, 1H), 6.04-5.94 (m, 1H), 5.82 (d, 1H, J=10.4 Hz), 5.59-5.51 (m, 1H), 5.10-4.91 (m, 1H), 4.89-4.62 (m, 2H), 4.42-4.23 (m, 2H), 4.03-3.84 (m, 2H), 3.76-3.58 (m, 1H), 3.56-3.39 (m, 1H), 3.31-3.20 (m, 2H), 3.19-3.00 (m, 5H), 2.77-2.60 (m, 2H), 2.42 (s, 6H), 2.13-2.02 (m, 2H).

### Example 8 Synthetic Route of Compound 8

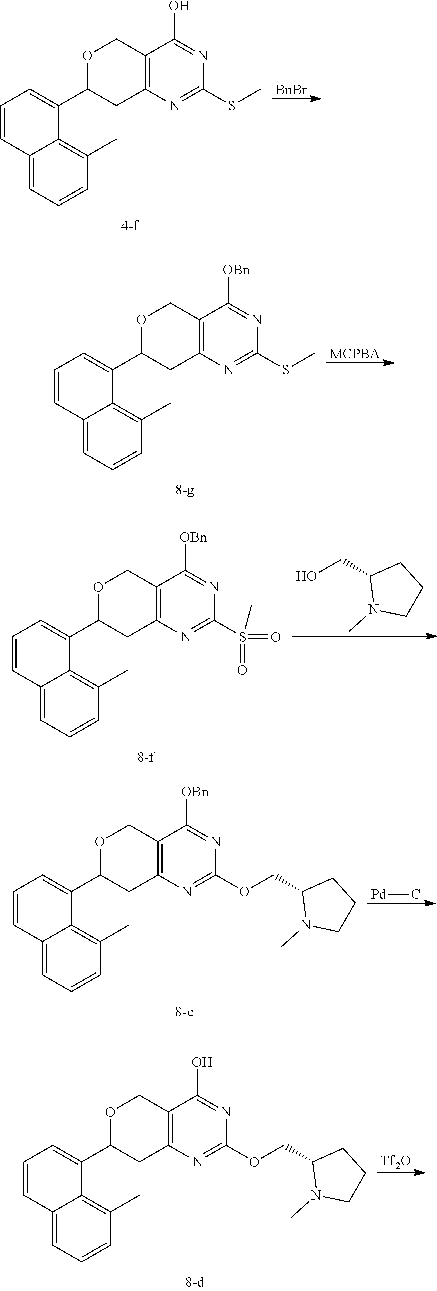

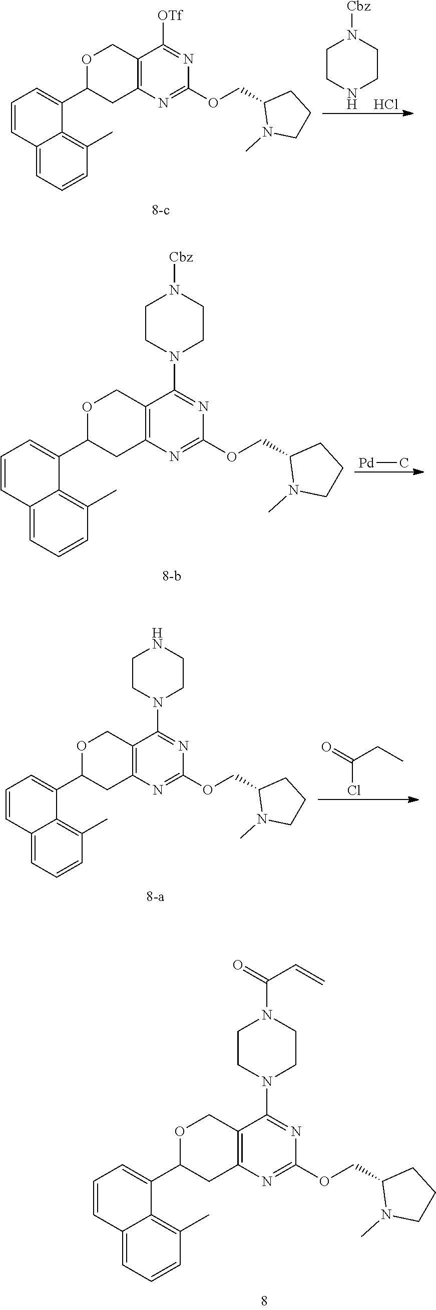

### Synthesis of Compound 8-g

At room temperature, compound 4-f (338 mg, 1.0 mmol) was dissolved in IMF (10 mL), and potassium carbonate (207 mg, 1.5 mmol) and benzyl bromide (132 μL, 1.1 mmol) were successively added. After stirred at room temperature for 3 hours, the reaction mixture was poured into 50 mL of water and extracted with ethyl acetate (50 mL*2). The organic phase was washed with saturated brine (100 mL*3), dried over anhydrous sodium sulfate, filtered and concentrated to give a crude product. The crude product was separated and purified through a flash column chromatography (PE/EA=2.1) to give compound (mixture) 8-g (211 mg, 49% yield) as a white solid. LC-MS (ESI): m/z=429.2 [M+1]⁻.

### Synthesis of Compound 8-f

Compound 8-g (211 mg, 0.49 mmol) was dissolved in ethyl acetate (20 mL) and added MCPBA (250 mg, 1.23 mmol) at room temperature. The mixture was stirred at room temperature for 3 hours and then quenched with a saturated sodium bicarbonate solution (50 mL) and extracted with ethyl acetate (50 mL*2). The organic phase was dried over anhydrous sodium sulfate, filtered and concentrated to give a crude product: the crude product was separated and purified through a flash column chromatography (EA/PE=1/1) to give compound 8-f (138 mg, 61% yield) as a white solid. LC-MS (ESI): m/z=461.0 [M+1]⁺.

### Synthesis of Compound 8-e

Compound 8-f (138 mg, 0.3 mmol) was dissolved in toluene (10 mL): in an ice-water bath, N-methyl-L-prolinol (65 μL, 0.54 mmol) and t-BuONa (58 mg, 0.6 mmol) were successively added. Under nitrogen atmosphere, the mixture was stirred in an ice-water bath for 0.5 hours and then quenched with water (30 mL) and extracted with ethyl acetate (50 mL*2). The organic phase was dried over anhydrous sodium sulfate, filtered and concentrated to give a crude product: the crude product was separated and purified thorough a flash column chromatography (MeOH/DCM=1/10) to give compound 8-e (105 mg, 70% yield) as a white solid. LC-MS (ESI): m/z=496.3 [M+H]⁺.

### Synthesis of Compound 8-d

Compound 8-e (105 mg, 0.212 mmol) was dissolved in methanol (30 mL), cooled to −78° C. replaced twice with nitrogen, and then added Pd/C (50 mg). After replaced three times with hydrogen, the reaction mixture was warmed to room temperature and stirred under hydrogen for 3 hours: the reaction mixture was then filtered and concentrated to give compound 8-d (91 mg, 100% yield) as a white solid. The crude product was used directly in the next reaction without further purification. LC-MS (ESI): m/z=406.1 [M+1]⁺.

### Synthesis of Compound 8-c

Compound 8-d (91 mg, 0.225 mmol) was dissolved in DCM (10 mL): under nitrogen atmosphere, in an ice-water bath. DIPEA (111 μL, 0.68 mmol) and trifluoromethanesulfonic anhydride (57 μL, 0.34 mmol) were successively added. The mixture was stirred in an ice-water bath for 1 hour and then quenched with a saturated sodium bicarbonate solution (20 mL) and extracted with DCM (50 mL*2). The organic phase was dried over anhydrous sodium sulfate, filtered and concentrated to give a crude product: the crude product was separated and purified through a flash column chromatography (MeOH/DCM=1/10) to give compound 8-c (68 mg, 56% yield) as a white solid. LC-MS (ESI): m/z=538.2 [M+1]⁺.

### Synthesis of Compound 8-b

At room temperature, compound 8-c (66 mg, 0.123 mmol) was dissolved in DMF (5 mL), and DIPEA (61 μL, 0.37 mmol) and 1-Cbz-piperazine hydrochloride (38 mg, 0.15 mmol) were successively added. Under nitrogen protection, the mixture was stirred at 100° C. for 1 hour and then cooled to room temperature, quenched with saturated brine (50 mL) and extracted with ethyl acetate (50 mL*2). The organic phase was washed with saturated brine (50 mL*3) and then dried over anhydrous sodium sulfate, filtered and concentrated to give a crude product: the crude product was separated and purified through a flash column chromatography (MeOH/DCM=1/10) to give compound 8-b (62 mg, 83 yield) as a white solid. LC-MS (ESI): m/z=608.2 [M+1]⁺.

### Synthesis of Compound 8-a

Compound 8-b (62 mg, 0.102 mmol) was dissolved in methanol (20 mL), cooled to −78° C. replaced twice with nitrogen and then added Pd/C (30 mg). After replaced three times with hydrogen, the reaction mixture was warmed to room temperature and stirred under hydrogen for 1 hour. The reaction mixture was filtered and concentrated to give compound 8-a (45 mg, 94% yield) as a white solid. The crude product was used directly in the next reaction without further purification. LC-MS (ESI): m/z=474.3 [M+1]⁺.

### Synthesis of Compound 8

At room temperature, compound 8-a (45 mg, 0.095 mmol) was dissolved in DCM (10 mL), and DIPEA (79 μL, 0.48 mmol) and acryloyl chloride (13 mg, 0.143 mmol) were successively added. Under nitrogen atmosphere, the mixture was stirred at room temperature for 20 hours and then quenched with saturated aqueous sodium bicarbonate solution (20 mL) and extracted with DCM (30 mL*2). The organic phase was dried over anhydrous sodium sulfate, filtered and concentrated to give a crude product: the crude product was subjected to PREP-TLC (MeOH/DCM=1/10) to give compound 8 (28 mg, 56% yield) as a white solid. LC-MS (ESI): m/z=528.3 [M+1]⁺: ¹H NMR (400 MHz, CDCl₃) δ 7.89-7.64 (m, 3H), 7.53-7.40 (m, 1H), 7.40-7.16 (m, 2H), 6.65-6.45 (m, 1H), 6.40-6.21 (m, 1H), 6.02-5.89 (m, 1H), 5.79-5.66 (m, 1H), 4.88-4.76 (m, 1H), 4.74-4.60 (m, 1H), 4.45-4.26 (m, 1H), 4.22-4.05 (m, 1H), 3.93-3.17 (m, 10H), 3.13-2.99 (m, 2H), 2.93 (s, 3H), 2.85-2.55 (m, 2H), 2.47 (s, 3H), 2.38-2.25 (m, 1H), 2.10-1.95 (m, 1H).

### Example 9 Synthetic Route of Compound 9

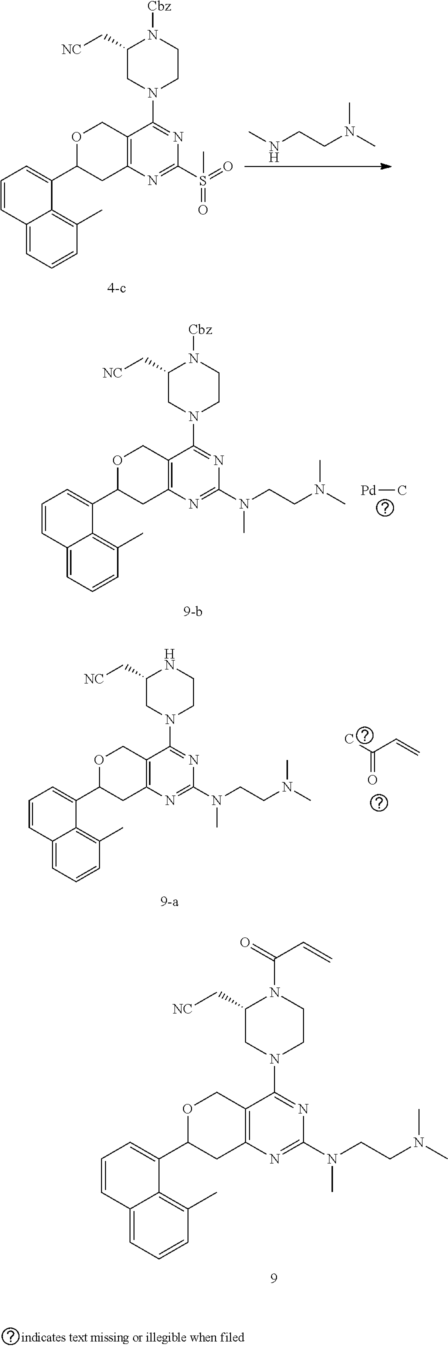

### Synthesis of Compound 9-b

At room temperature, compound 4-c (61 mg, 0.1 mmol) was dissolved in dioxane (5 mL) and then added N,N-dimethyl-N′-methylethylenediamine (255 μL, 2 mmol). Under nitrogen atmosphere, the reaction mixture was stirred at 110° C. for 24 hours, and the reaction mixture was then cooled to room temperature and concentrated to dryness to give a crude product. The crude product was separated and purified through a flash column chromatography (MeOH/DCM=1/10) to give compound 9-b (49 mg, 78% yield) as a white solid. LC-MS (ESI): m/z=634.4 [M+1]⁺.

### Synthesis of Compound 9-a

Compound 9-b (69 mg, 0.11 mmol) was dissolved in methanolic ammonia (7 M, 50 mL), cooled to −78° C. replaced twice with nitrogen, and then added Pd/C (50 mg). After replaced three times with hydrogen, the reaction mixture was warmed to room temperature and stirred under hydrogen for 1 hour. The reaction mixture was filtered and concentrated to give compound 9-a (50 mg, 99% yield) as a white solid. LC-MS (ESI): m/z=500.5 [M+1]⁺.

### Synthesis of Compound 9

At room temperature, compound 9-a (50 mg, 0.1 mmol) was dissolved in DCM (10 mL), and DIPEA (83 μL, 0.5 mmol) and acryloyl chloride (15 mg, 0.15 mmol) were successively added. Under nitrogen atmosphere, the mixture was stored at room temperature for 20 hours and then quenched with a saturated sodium bicarbonate solution (20 mL) and extracted with DCM (50 mL*2). The organic phase was dried over anhydrous sodium sulfate, filtered and concentrated to give a crude product. The crude product was separated and purified through a flash column chromatography (MeOH/DCM=1/10) to give compound 9 (27 mg, 49% yield) as a white solid. LC-MS (ESI): m/z=554.4 [M+1]⁺; ¹H NMR (400 MHz, CDCl₃): δ 7.87-7.69 (m, 3H), 7.55-7.42 (m, 1H), 7.40-7.29 (m, 2H), 6.67-6.45 (m, 1H), 6.37 (d, 1H, J=16.8 Hz), 5.96 (d, 1H, J=7.2 Hz), 5.80 (d, 1H, J=10.4 Hz), 5.20-4.39 (m, 3H), 3.89 (d, 1H, J=13.2 Hz), 3.81-3.65 (m, 2H), 3.65-3.50 (m, 1H), 3.44-3.25 (m, 1H), 3.20-3.03 (m, 4H), 3.01-2.85 (m, 5H), 2.78-2.61 (m, 1H), 2.58-2.41 (m, 2H), 2.31 (s, 6H), 2.14-1.83 (m, 4H).

### Example 10 Synthetic Route of Compound 10

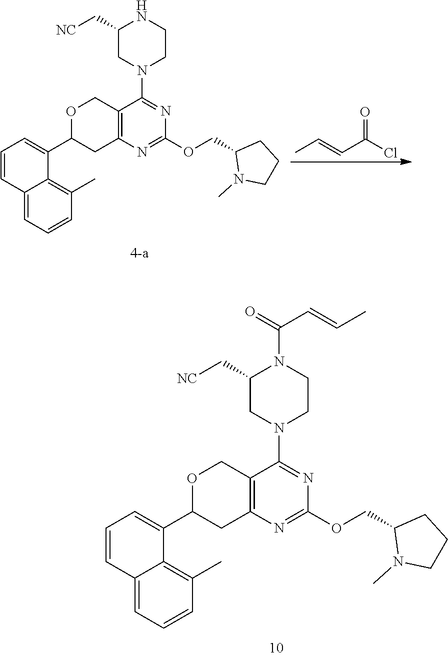

### Synthesis of Compound 4-a

Compound 4-a (61 mg) was prepared according to the synthetic route of compound 4.

### Synthesis of Compound 10

At room temperature, compound 4-a (61 mg, 0.12 mmol) was dissolved in DCM (10 mL), and DIPEA (99 μL, 0.6 mmol) and 2-butenoyl chloride (17 μL, 0.18 mmol) were successively added. Under nitrogen atmosphere, the mixture was stirred at room temperature for 20 hours and then quenched with a saturated sodium bicarbonate solution (20 mL) and extracted with DCM (50 mL*2). The organic phase was dried over anhydrous sodium sulfate, filtered and concentrated to give a crude product: the crude product was separated and purified through a flash column chromatography (MeOH/DCM=1/10) to give compound 10 (55 mg, 78% yield) as a white solid. LC-MS (ESI): m/z=581.3 [M+1]⁺; ¹H NMR (500 MHz, CDCl₃): δ 7.87-7.68 (m, 3H), 7.54-7.41 (m, 1H), 7.40-7.29 (m, 2H), 7.05-6.90 (m, 1H), 6.30-6.18 (m, 1H), 6.04-5.88 (m, 1H), 5.12-4.91 (m, 1H), 4.89-4.57 (m, 2H), 4.44-4.29 Om 1H), 4.22-4.10 (m, 1H), 4.02-3.76 (m, 2H), 3.75-3.54 (m, 1H), 3.51-3.34 (m, 1H), 3.31-3.15 (m, 2H), 3.15-2.99 (m, 3H), 2.99-2.82 (m, 4H), 2.81-2.56 (m, 3H), 2.47 (s, 3H), 2.35-2.13 (m, 1H), 2.12-1.97 (m, 1H), 1.91 (s, 3H), 1.85-1.82 (m, 2H).

### Example 11 Synthetic Route of Compounds 11-1 and 11-2

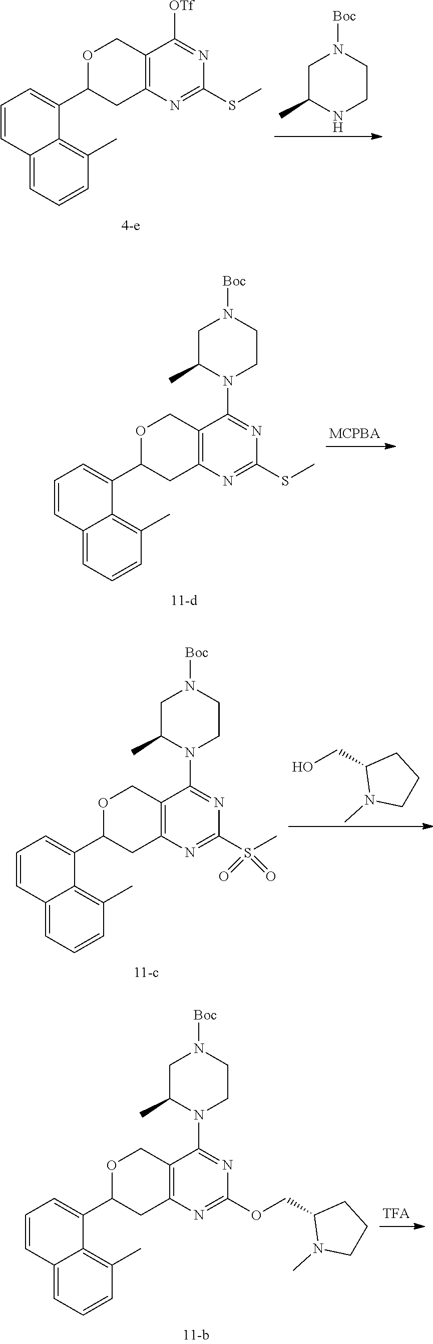

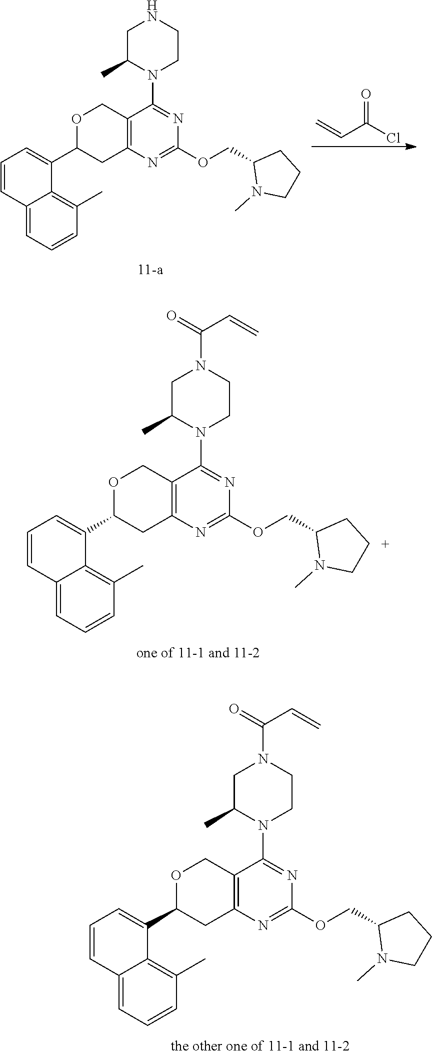

### Synthesis of Compound 11-d

At room temperature, to a solution of 4-e (220 mg, 0.47 mmol) and (S)-4-N-tert-butoxycarbonyl-2-methylpiperazine (112 mg, 0.56 mmol) in DMF (10 mL) was added DIPEA (121 mg, 0.94 mmol). The reaction temperature was increased to 100° C., and the reaction was stirred at this temperature for 1 hour. The reaction mixture was cooled to room temperature, added water and extracted with ethyl acetate (30 mL*2); the combined organic phase was dried over anhydrous sodium sulfate, filtered and subjected to rotary evaporation: the crude product was separated and purified through a flash column chromatography (EA:PE=0:100 to 30:70) to give compound 11-d (173 mg, 71%) as a white solid. LC-MS (ESI): m/z=521.3 [M+H]⁺.

### Synthesis of Compound 11-c

In an ice bath, to a solution of 11-d (173 mg, 0.33 mmol) in ethyl acetate (10 mL) was added 85% m-chloroperoxybenzoic acid (169 mg, 0.83 mmol). The reaction was slowly warmed to room temperature, stirred for 3 hours and then added saturated aqueous sodium bicarbonate solution and extracted with ethyl acetate (50 mL); the organic phase was dried over anhydrous sodium sulfate, filtered and subjected to rotary evaporation; the crude product was separated and purified through a flash column chromatography (EA:PE=0:100 to 50; 50) to give compound 11-c (154 mg, 84%) as a white solid. LC-MS (ESI): m/z=553.2 [M+H]⁺.

### Synthesis of Compound 11-b

At room temperature, to a solution of 11-c (154 mg, 0.28 mmol) in toluene (6 mL) were respectively added a solution of N-methyl-L-prolinol (48 mg, 0.42 mmol) in toluene (4 mL) and sodium tert-butoxide (53 mg, 0.56 mmol). The mixture was stirred at room temperature for 3 hours and then concentrated, added water and extracted with ethyl acetate (30 mL*2); the combined organic phase was dried over anhydrous sodium sulfate, filtered and subjected to rotary evaporation; the crude product was separated and purified through a flash column chromatography (DCM:MeOH=10:1) to give compound 11-b (120 mg, 73%) as a white solid. LC-MS (ESI): m/z=588.3 [M+H]⁺.

### Synthesis of Compound 11-a

At room temperature, to a solution of 11-b (120 mg, 0.2 mmol) in dichloromethane (8 mL) was added TFA (2 mL). The mixture was stirred at room temperature overnight and then concentrated, added saturated aqueous sodium bicarbonate solution and extracted with ethyl acetate (30 μL*2); the combined organic phase was dried over anhydrous sodium sulfate, filtered and subjected to rotary evaporation to give compound 11-a (70 mg, 70%) as a white solid. LC-MS (ESI): m/z=488.0 [M+H]⁺.

### Synthesis of Compounds 11-1 and 11-2

In an ice bath, to a solution of 11-a (70 mg, 0.14 mmol) in dichloromethane (5 mL) were respectively added acryloyl chloride (19 mg, 0.22 mmol) and DIPEA (36 mg, 0.28 mmol). The reaction temperature was increased to room temperature, and the reaction was stirred at room temperature for 2 hours and then concentrated, added water and extracted with ethyl acetate (30 mL*2); the combined organic phase was dried over anhydrous sodium sulfate, filtered and subjected to rotary evaporation: the crude product was subjected to chiral resolution to give compound 11-1 (25 mg, 32%) as a white solid and 11-2 (20 mg, 26%) as a white solid.

| Chiral analysis conditions | Chiral preparation conditions |
|---|---|
| instrument: SFC Method Station | instrument: SFC-150 (Waters) |
| (Thar, Waters) | chromatographic column: |
| chromatographic column: | R.R-WHELK-O1 20 * 250 |
| R.R-WHELK-O1 4.6 * 100 mm, | mm, 10 um (REGIS) |
| 5 μm (REGIS) | column temperature: 35° C. |
| column temperature: 40° C. | mobile phase: |
| mobile phase: CO₂/(MeOH/CAN = | CO₂/(MeOH/CAN = |
| 3:2 (0.1% TEA)) = 55/45 | 1:1(0.1% TEA)) = 40/60 |
| flow rate: 4.0 ml/min | flow rate: 120 g/min |
| detection wavelength: 254 nm | column pressure: 100 bar |
| | detection wavelength: 214 |
| | nm |
| | cycling time: 7.5 min |
| | sample solution: 70 mg |
| | dissolved in 20 ml of |
| | methanol |
| 11-1: retention time: 3.26; | |
| d.e. % = 100% | |
| 11-2: retention time: 4.16; | |
| d.e. % = 98.6% | |

11-1: LC-MS (ESI): m/z=542.3 [M+H]⁺; ¹H NMR (500 MHz, CDCl₃): δ 7.85 (t, J=8 Hz, 2H), 7.78 (dd, J=8.2 Hz, 1H), 7.50 (t, J=7.5 Hz, 1H), 7.40-7.35 (m, 2H), 6.66-6.53 (m, 1H), 6.39 (dd, J=17.2 Hz, 1H), 6.00 (dd, J=10, 3.5 Hz, 1H), 5.78 (d, J=9 Hz, 1H), 4.85-4.80 (m, 1H), 4.77-4.70 (m, 1H), 4.43-4.16 (m, 311), 4.07-3.96 (m, 1H), 3.77-3.57 (m, 1H), 3.49-3.43 (m, 1H), 3.32-3.24 (m, 2H), 3.14-3.03 (m, 2H), 2.96 (s, 3H), 2.73-2.66 (m, 1H), 2.49 (s, 3H), 2.34-2.24 (m, 1H), 2.11-2.02 (m, 1H), 1.88-1.73 (m, 311), 1.37-1.31 (m, 1H), 1.24-1.14 (m, 4H).

11-2: LC-MS (ESI): m/z=542.3 [M+H]⁺: ¹H NMR (500 MHz, CDCl₃): δ 7.83 (dd, J=17, 8 Hz, 2H), 7.78 (dd, J=7.2 Hz, 1H), 7.49 (t. J=7.5 Hz, 1H), 7.40-7.35 (m, 2H), 6.66-6.53 (m, 1H), 6.39 (dd, J=17, 1.5 Hz, 1H), 6.00 (d, J=7.5 Hz, 1H), 5.77 (d, J=9.5 Hz, 1H), 4.88 (d, J=14 Hz, 1H), 4.68 (d, J=13.5 Hz, 1H), 4.45-4.34 (m, 1H), 4.20-4.09 (m, 1H), 4.00-3.88 (m, 1H), 3.80-3.69 (m, 1H), 3.62-3.44 (m, 1H), 3.34-3.25 (m, 2H), 3.14-3.03 (m, 3H), 2.96 (s, 3H), 2.73-2.65 (m, 1H), 2.49 (s, 3H), 2.34-2.24 (m, 1H), 2.12-2.02 (m, 1H), 1.88-1.72 (m, 3H), 1.36-1.28 (m, 5H).

### Example 12 Synthetic Route of Compound 12

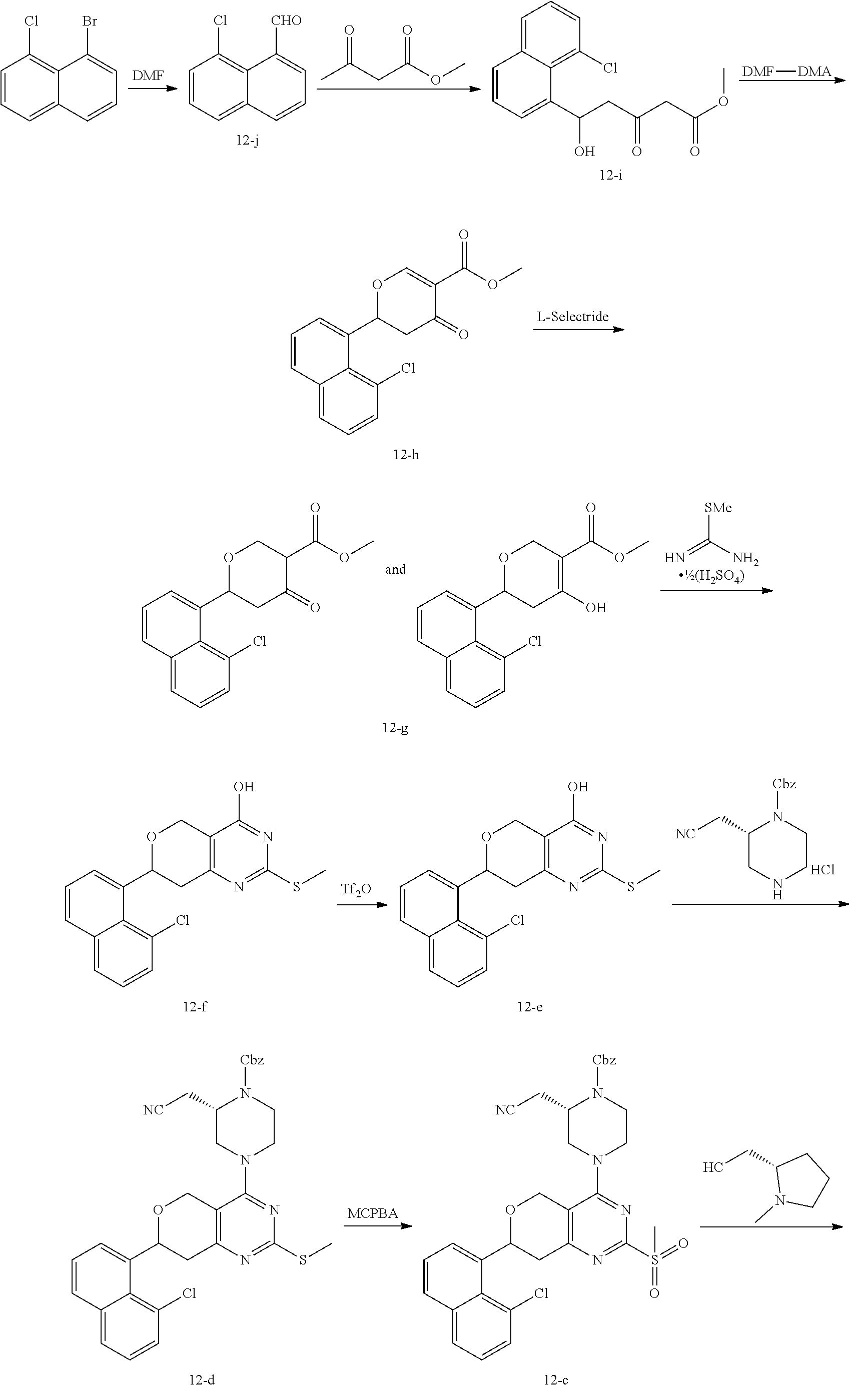

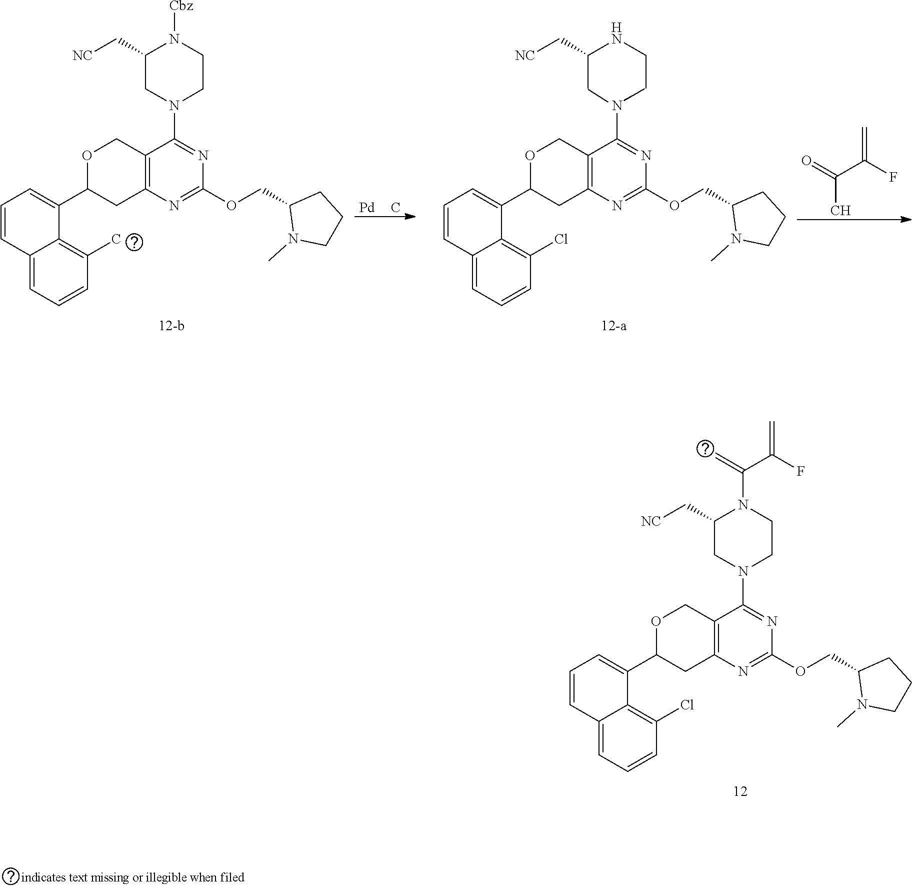

### Synthesis of Compound 12-j

Compound 1-promo-8-chloronaphthalene (500 mg, 2.07 mmol) was dissolved in THE (20 mL), cooled to −78° C., and added dropwise n-BuLi (2.5 M, 1.66 mL, 4.14 mmol) under nitrogen protection. After completion of the dropwise addition, the mixture was stirred at −78° C. for 10 minutes, and then DMF (800 μL, 10.35 mmol) was added dropwise at −78° C. After completion of the addition, the reaction mixture was stirred at −78° C. for 30 minutes and then warmed to room temperature and stirred for 2 hours: the reaction was quenched with 50 mL of saturated ammonium chloride solutions and extracted with ethyl acetate (50 mL*2). The organic phase was washed with saturated brine (50 mL*2), dried over anhydrous sodium sulfate, filtered and concentrated to give a crude product. The crude product was separated and purified through a flash column chromatography (EA/PE=1/10) to give compound 12-j (330 mg, 84% yield) as a white solid. LC-MS (ESI): m/z=191.0 [M+H]⁺: ¹H NMR (400 MHz, CDCL₃): δ 11.31 (s, 1H), 8.03 (dd, 1H, J₁=1.2 Hz, J₂=8.4 Hz), 7.92 (dd, 1H, J₁=1.2 Hz, J₂=7.2 Hz), 7.86 (1H, J=8.4 Hz), 7.70 (dd, 1H, J₁=1.2 Hz, J₂=7.6 Hz), 7.59 (t, 1H, J=7.6 Hz), 7.47 (t, 1H, J=8 Hz).

### Synthesis of Compound 12-i

At room temperature, NaH (60%, 242 mg, 6.05 mmol) was added to 6 mL of THF. Under nitrogen atmosphere, methyl acetoacetate (543 μL, 5.04 mmol) was then added at room temperature: under nitrogen atmosphere, the mixture was stirred at room temperature for 30 minutes and then added dropwise n-BuLi (2.5 M, 2.4 mL, 6.05 mmol) at −15° C., to −10° C. After completion of the addition, the mixture was kept at this temperature for 30 minutes, and then a solution of compound 12-j (320 mg, 1.68 mmol) in THF (10 mL) was added dropwise. After completion of the addition, the mixture was stirred at low temperature (−10° C., to 0° C.) for 2 hours: the reaction was then quenched with a saturated ammonium chloride solution (50 mL) and extracted with ethyl acetate (50 mL*2). The organic phase was washed with saturated brine (50 mL*2), dried over anhydrous sodium sulfate, filtered and concentrated to give a crude product: the crude product was separated and purified through a flash column chromatography (EA/DCM=110) to give compound 12-i (510 mg, 99% yield) as a white solid. LC-MS (ESI): m/z=329.1 [M+Na]⁺: ¹H NMR (400 MHz, CDCl₃): δ 8.06 (d, 1H, J=6.4 Hz), 7.79 (d, 2H, J=8 Hz), 7.58 (dd, 1H, J₁=7.6 Hz, J₂=1.6 Hz), 7.53 (t, 1H, J=7.6 Hz), 7.34 (t, 1H, J=7.6 Hz), 6.91 (dd, 1H, J₁=9.2 Hz, J₂=2.4 Hz), 3.74 (s, 3H), 3.54 (s, 2H), 3.36 (dd, 1H, J₁=18 Hz, J₂=1.6 Hz), 3.24 (d, 1H, J=3.6 Hz), 2.85-2.75 (m, 1H).

### Synthesis of Compound 12-h

At room temperature, compound 12-i (510 mg, 1.66 mmol) was dissolved in DCM (18 mL), and then under nitrogen atmosphere. DMF-DMA (245 μL, 1.83 mmol) was added at room temperature. At room temperature, the reaction mixture was stirred for 45 minutes and then was added BF₃.Et₂O (232 μL, 1.83 mmol). After completion of the addition, the mixture was stirred at room temperature for 1 hour and then diluted with 100 mL of ethyl acetate. The organic phase was washed successively with a saturated NaHCO₃ solution (100 mL) and saturated brine (100 mL*2), dried over anhydrous sodium sulfate, filtered and concentrated to give crude product compound 12-h (520 mg). The crude product was used directly in the next reaction without purification. LC-MS (ESI): m/z=317.1 [M+1]⁺.

### Synthesis of Compound 12-g

At room temperature, compound 12-h (520 mg, 1.64 mmol) was dissolved in THF (20 mL): at −78° C., under nitrogen atmosphere, tri-sec-butyl lithium borohydride (1 M, 1.64 mL, 1.64 mmol) was then added dropwise. After completion of the addition, the mixture was stirred at −78° C. for 1 hour; the reaction was then quenched by adding a saturated ammonium chloride solution (50 mL) and extracted with ethyl acetate (50 mL*2); the organic phase was washed with saturated brine (50 mL*2), dried over anhydrous sodium sulfate, filtered and concentrated to give a crude product: the crude product was separated and purified through a flash column chromatography (PE/EA=4/1) to give compound 12-g (338 mg, 65% yield) as a yellow oil. LC-MS (ESI): m/z=319.0 [M+1]⁺.

### Synthesis of Compound 12-f

At room temperature, compound 12-g (338 mg, 1.06 mmol) was dissolved in methanol (20 mL): at 0° C. under nitrogen atmosphere, sodium methylate (286 mg, 5.3 mmol) and compound 2-methyl-2-thiourea sulfate (265 mg, 0.954 mmol) were successively added. After completion of the addition, the mixture was warmed to room temperature and stirred for 20 hours. The pH of the reaction mixture was adjusted to 5 with 1 N dilute hydrochloric acid: the solid was precipitated out and filtered: the filter cake was washed with water (5 mL*2) to collect solid, which was dried in vacuum to give crude product 12-f (313 mg) as a white solid. LC-MS (ESI): m/z=359.1 [M+1]⁺.

### Synthesis of Compound 12-e

At room temperature, compound 12-f (313 mg, 0.87 mmol) was dissolved in DCM (10 mL): in an ice-water bath, under nitrogen atmosphere, DIPEA (431 μL, 2.61 mmol) and trifluoromethanesulfonic anhydride (219 μL, 1.31 mmol) were then successively added. After completion of the addition, the reaction mixture was stirred in an ice-water bath for 2 hours and then quenched with a saturated sodium bicarbonate solution (50 mL) and extracted with DCM (50 mL*2); the organic phase was dried over anhydrous sodium sulfate, filtered and concentrated to give a crude product: the crude product was separated and purified through a flash column chromatography (EA/PE=1/10) to give compound 12-e (83 mg, 16°/s yield for two steps) as a white solid. LC-MS (ESI): m/z=491.0 [M+1]⁺.

### Synthesis of Compound 12-d

At room temperature, compound 12-e (83 mg, 0.169 mmol) was dissolved in DMF (10 mL), and then DIPEA (84 μL, 0.507 mmol) and benzyl (S)-2-cyanomethylpiperazine-1-carboxylate hydrochloride (59.9 mg, 0.203 mmol) were successively added. After completion of the addition, under nitrogen protection, the mixture was stirred at 100° C. for 1 hour and then cooled to room temperature: the reaction was quenched with saturated brine (50 mL) and extracted with ethyl acetate (50 mL*2). The organic phase was washed with saturated brine (50 mL*3) and then dried over an sodium sulfate, filtered and concentrated to give a crude product: the crude product was separated and purified through a flash column chromatography (EA/PE=1/1) to give compound 12-d (101 mg, 99% yield) as a white solid. LC-MS (ESI): m/z=600.2 [M+1]⁺.

### Synthesis of Compound 12-c

At room temperature, compound 12-d (101 mg, 0.168 mmol) was dissolved in ethyl acetate (10 mL), and MCPBA (85%, 88.4 mg, 0.437 mmol) was then added at room temperature. After completion of the addition, the mixture was stirred at room temperature for 2 hours and then quenched with a saturated sodium bicarbonate solution (20 mL) and extracted with ethyl acetate (25 mL*2); the organic phase was dried over anhydrous sodium sulfate, filtered and concentrated to give a crude product: the crude product was separated and purified through a flash column chromatography (EA/PE=1/4) to give compound 12-c (88 mg, 82% yield) as a white solid. LC-MS (ESI): m/z=632.1 [M+1]⁺.

### Synthesis of Compound 12-b

At room temperature, compound 12-c (88 mg, 0.139 mmol) was dissolved in toluene (10 mL), and then the reaction mixture was cooled to 0° C.: N-methylprolinol (29 μL, 0.243 mmol) and t-BuONa (27 mg, 0.278 mmol) were successively added. After completion of the addition, under nitrogen atmosphere, the reaction mixture was stirred in an ice-water bath for 0.5 hours and then quenched with water (20 mL) and extracted with ethyl acetate (30 mL*2). The organic phase was dried over anhydrous sodium sulfate, filtered and concentrated to give a crude product: the crude product was separated and purified through a flash column chromatography (MeOH/DCM=1710) to give compound 12-b (78 mg, 84% yield) as a white solid. LC-MS (ESI): m/z=667.3 [M+1]⁺;

### Synthesis of Compound 12-a

At room temperature, compound 12-b (72 mg, 0.108 mmol) was dissolved in methanol (50 mL); the reaction mixture was then cooled to −78° C., replaced twice with nitrogen, and then added Pd/C (150 mg) and ZnBr₂ (24.3 mg, 0.108 mmol) and replaced three times with hydrogen: the reaction mixture was warmed to room temperature and stirred under hydrogen for 5 hours. The reaction mixture was filtered and concentrated to give a crude product: the crude product was separated and purified through a flash column chromatography (MeOH/DCM=1:4) to give compound 12-a (20 mg, 35% yield) as a white solid. LC-MS (ESI): m/z=533.0 [M+1]⁺.

### Synthesis of Compound 12

At room temperature, compound 2-fluoroacrylic acid (5.1 mg, 0.0563 mmol) was dissolved in DMF (2 mL): at 0° C. HATU (25.6 mg, 0.0675 mmol) and DIPEA (18.6 μL, 0.113 mmol) were then successively added: after completion of the addition, under nitrogen atmosphere, the reaction mixture was stirred at 0° C. for 20 minutes, and a solution of compound 12-a (20 mg, 0.0375 mmol) in DMF (3 mL) was then added to the above-mentioned reaction mixture, which was warmed to room temperature and continuously stirred for 5 hours. The reaction was quenched with saturated brine (20 mL) and extracted with ethyl acetate (25 mL*2); the organic phase was washed with saturated brine (50 mL*3), dried over anhydrous sodium sulfate, filtered and concentrated to give a crude product: the crude product was separated and purified by PREP-TLC (MeOH/DCM=1/10) to give compound 12 (6 mg, 26% yield) as a white solid. LC-MS (ESI): m/z=605.2 [M+1]⁺: ¹H NMR (400 MHz, CDCl₃): δ 7.99-7.93 (m, 1H), 7.83 (t, 2H, J=8.8 Hz), 7.62-7.49 (m, 2H), 7.36 (t, 1H, J=7.6 Hz), 6.55-6.44 (m, 1H), 5.51-5.31 (m, 1H), 5.25 (d, 1H, J=16.8 Hz), 5.02-4.93 (m, 1H), 4.82 (dd, 1H, J₁=2.4 Hz, J₂=13.6 Hz), 4.48-4.38 (m, 1H), 4.32-4.19 (m, 1H), 4.17-4.04 (m, 1H), 4.00 (d, 1H, J=14 Hz), 3.87-3.70 (m, 1H), 3.66-3.36 (m, 2H), 3.31-3.16 (m, 2H), 3.14-2.98 (m, 1H), 2.96-2.69 (m, 4H), 2.59 (d, 3H, J=18 Hz), 2.52-2.34 (m, 1H), 2.15-2.06 (m, 1H), 1.87-1.74 (m, 2H), 0.93-0.76 (m, 2H).

### Synthesis of Compounds 12-1 and 12-2

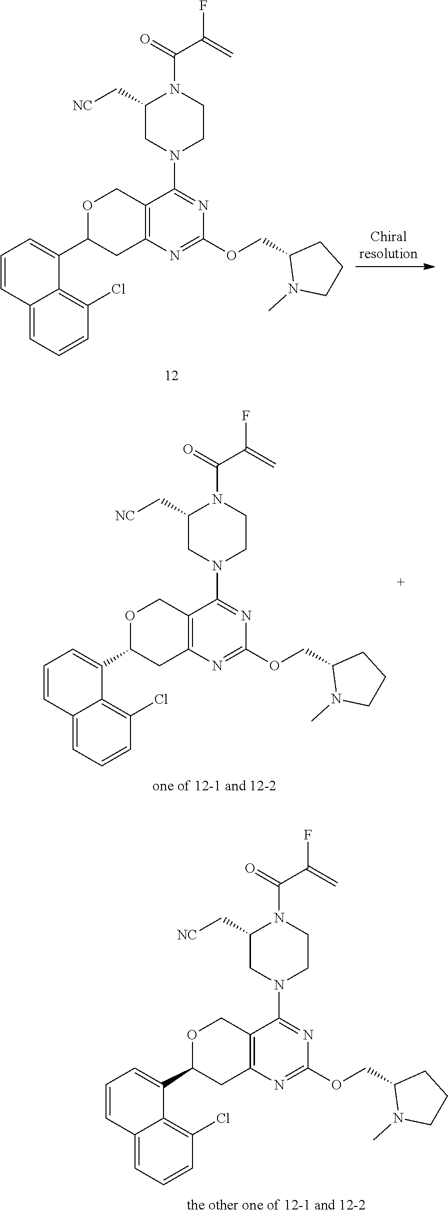 [SMILES: C=C(F)C(=O)N1CCN(c2nc(OC[C@@H]3CCCN3C)nc3c2COC(c2cccc4cccc(Cl)c24)C3)C[C@@H]1CC#N ; C=C(F)C(=O)N1CCN(c2nc(OC[C@@H]3CCCN3C)nc3c2CO[C@@H](c2cccc4cccc(Cl)c24)C3)C[C@@H]1CC#N ; C=C(F)C(=O)N1CCN(c2nc(OC[C@@H]3CCCN3C)nc3c2CO[C@H](c2cccc4cccc(Cl)c24)C3)C[C@@H]1CC#N]

The newly-prepared compound 12 (260 mg, 0.43 mmol) was subjected to chiral resolution to give compound 12-1 (76 mg, 29% yield) as a white solid and compound 12-2 (67 mg, 26% yield) as a white solid.

| Chiral analysis conditions | Chiral preparation conditions |
|---|---|
| equipment: SFC Method Station | instrument: SFC-150 (Thar, |
| (Thar, Waters) | Waters) |
| chromatographic column: OJ-H | chromatographic column: OJ |
| 4.6 * 100 mm, 5 μm (Daicel) | 20 * 250 mm, 10 μm (Daicel) |
| column temperature: 40° C. | column temperature: 35° C. |
| mobile phase: CO₂/MeOH (0.1% | mobile phase: CO₂/MEOH |
| TEA) = 60/40 | (0.1% TEA) = 40/60 |
| flow rate: 4.0 ml/min | flow rate: 120 g/min |
| wavelength: 254 nm | back pressure: 100 bar |
| back pressure: 120 bar | detection wavelength: 214 nm |
| | cycling time: 6.0 min |
| 12-1: retention time: 1.36 min; | |
| d.e. % = 100%; | |
| 12-2: retention time: 2.77 min; | |
| d.e. % = 97.5%. | |

12-1: LC-MS (ESI): m/z=605.3 [M+1]⁺: ¹HNMR (400 MHz, CDCl₃) δ 7.96 (d, 1H, J=7.2 Hz), 7.83 (t, 2H, J=8.4 Hz), 7.65-7.50 (m, 2H), 7.36 (t, 1H, J=8.0 Hz), 6.47 (dd, 1H, =10.8 Hz, J₂=3.2 Hz), 5.42 (d, 1H, J=49.2 Hz), 5.26 (dd, 1H, J₁=3.6 Hz, J₂=16.8 Hz), 5.05-4.76 (m, 1H), 4.97 (d, 1H, J=13.6 Hz), 4.84 (d, 1H, J=13.6 Hz), 4.36 (dd, 1H, J₁=4.8 Hz, J₂=10.4 Hz), 4.17 (dd, 1H, J₁=6.8 Hz, J₂=10.8 Hz), 4.06-3.87 (m, 1H), 3.77 (d, 1H, J=10 Hz), 3.59 (dd, 1H, J₁=2.4 Hz, J₂=17.6 Hz), 3.50-3.15 (m, 3H), 3.14-2.99 (m, 2H), 2.96-2.82 (m, 2H), 2.72-2.59 (m, 1H), 2.47 (s, 3H), 2.32-2.21 (m, 1H), 2.10-1.98 (m, 1H), 1.89-1.67 (m, 4H).

12-2: LC-MS (ESI): m/z=605.2 [M+1]⁺: ¹HNMR (400 MHz, CDCl₃) δ 7.97 (d, 1H, J=7.2 Hz), 7.83 (t, 2H, J=9.2 Hz), 7.63-7.51 (m, 2H), 7.36 (t, 1H, J=7.6 Hz), 6.52 (dd, 1H, J₁=3.2 Hz, J₂=10.8 Hz), 5.42 (d, 1H, J=47.2 Hz), 5.25 (dd, 1H, J₁=3.6 Hz, J₂=16.4 Hz), 4.99 (d, 1H, J=14.0 Hz), 4.82 (d, 1H, J=13.6 Hz), 5.05-4.72 (m, 1H), 4.38 (dd, 1H, J₁=4.8 Hz, J₂=10.4 Hz), 4.15 (dd, 1H, =6.8 Hz, =10.8 Hz), 3.98 (d, 1H, J=14 Hz), 3.87-3.73 (m, 1H), 3.60 (dd, 1H, J₁=2.4 Hz, J₂=18.4 Hz), 3.66-3.54 (m, 1H), 3.54-3.41 (m, 1H), 3.16-2.98 (m, 2H), 2.95-2.71 (m, 3H), 2.71-2.61 (m, 1H), 2.46 (s, 3H), 2.33-2.19 (m, 1H), 2.10-1.98 (m, 1H), 1.90-1.66 (m, 4H).

### Example 13 Synthetic Route of Compound 13

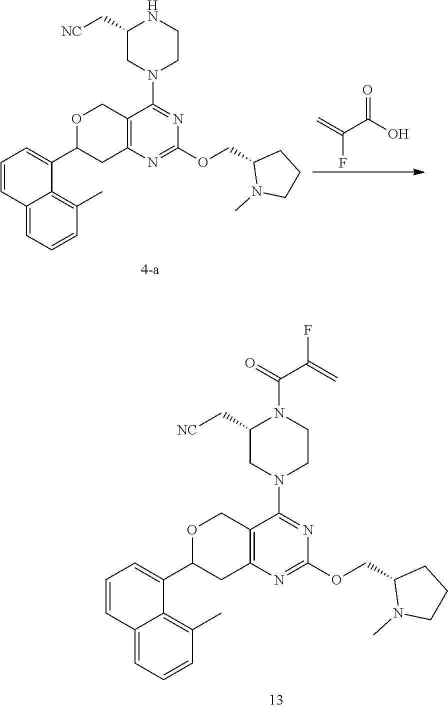

### Synthesis of Compound 13

At room temperature, to a solution of 4-a (240 mg, 0.47 mmol) in DMF (5 mL) were respectively added HATU (356 mg, 0.94 mmol), N,N-diisopropylethylamine (0.23 mL, 1.40 mmol) and 2-fluoroacrylic acid (63.2 mg, 0.70 mmol). The reaction mixture was stirred at room temperature overnight. After completion of the reaction, the solid was precipitated out by adding water and filtered; the filter cake was washed with water and dried to give a crude product, which was then purified by silica gel column chromatography (DCM/MeOH 10/1) to give product 13 (220 mg, 80%) as a white foamy solid. LC-MS (ESI): m/z=585.0 [M+H]⁺; ¹H NMR (400 MHz, CDCl₃): δ 7.78-7.83 (m, 2H), 7.71-7.77 (m, 1H), 7.45-7.50 (m, 1H), 7.33-7.38 (m, 2H), 6.00-6.07 (m, 1H), 5.19-5.25 (m, 1H), 4.89-4.97 (m, 1H), 4.66-4.72 (m, 2H), 4.54-4.60 (m, 2H), 3.98-4.08 (m, 1H), 3.90-3.98 (m, 1H), 3.75-3.89 (m, 1H), 3.58-3.75 (m, 2H), 3.44-3.58 (m, 1H), 3.04 (d, 3H, J=4.4 Hz), 2.96-3.37 (m, 6H), 2.93 (s, 3H), 2.65-2.86 (m, 2H), 2.26-2.42 (m, 2H), 2.07-2.24 (m, 2H).

### Synthesis of Compounds 13-1 and 13-2

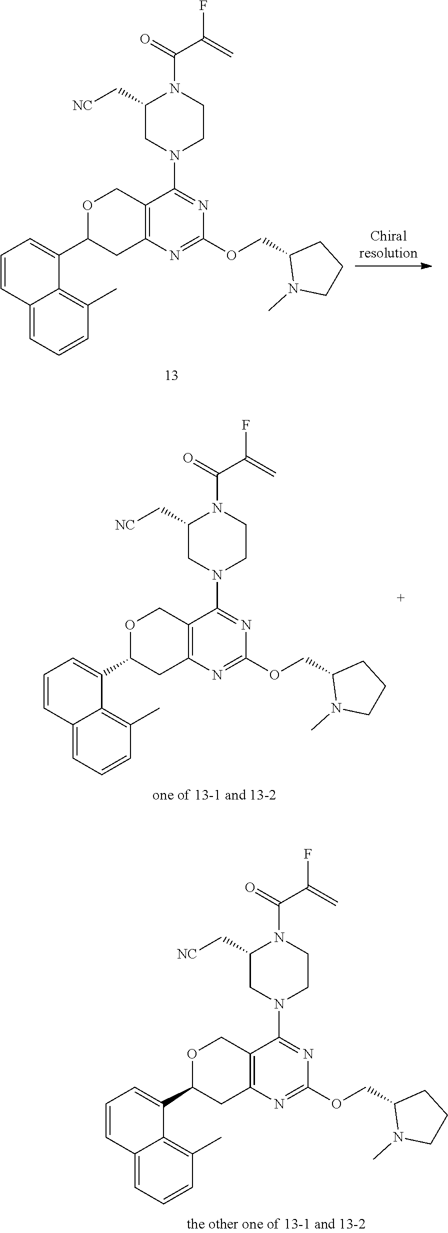 [SMILES: C=C(F)C(=O)N1CCN(c2nc(OC[C@@H]3CCCN3C)nc3c2COC(c2cccc4cccc(C)c24)C3)C[C@@H]1CC#N ; C=C(F)C(=O)N1CCN(c2nc(OC[C@@H]3CCCN3C)nc3c2CO[C@@H](c2cccc4cccc(C)c24)C3)C[C@@H]1CC#N ; C=C(F)C(=O)N1CCN(c2nc(OC[C@@H]3CCCN3C)nc3c2CO[C@H](c2cccc4cccc(C)c24)C3)C[C@@H]1CC#N]

Compound 13 (200 mg, 0.34 mmol) was purified by chiral resolution to give compound 13-1 (70 mg, 35%) as a white solid and compound 13-2 (71 mg, 36%) as a white solid.

| Chiral analysis conditions | Chiral preparation conditions |
|---|---|
| equipment: SFC Method Station | instrument: SFC-150 (Thar, |
| (Thar, Waters) | Waters) |
| chromatographic column: OJ-H | chromatographic column: OJ |
| 4.6 * 100 mm, 5 μm (Daicel) | 20 * 250 mm, 10 μm (Daicel) |
| column temperature: 40° C. | column temperature: 35° C. |
| mobile phase: CO₂/MeOH (0.1% | mobile phase: |
| TEA) = 60/40 | CO₂/[MeOH:CAN = 1:1(0.1% |
| flow rate: 4.0 ml/min | TEA)] = 60/40 |
| wavelength: 254 nm | flow rate: 100 g/min |
| back pressure: 120 bar | back pressure: 100 bar |
| | detection wavelength: 214 nm |
| | cycling time: 4.0 min |
| 13-1: retention time: 1.17 min; | |
| d.e. % = 100%; | |
| 13-2: retention time: 2.76 min; | |
| d.e. % = 99.4%. | |

13-1: LC-MS (ESI): m/z=585.0 [M+1]⁺: ¹H NMR (400 MHz, CDCl₃): δ 7.83 (d, 1H, J=8.4 Hz), 7.74-7.77 (m, 2H), 7.46 (t, 1H, J=8.0 Hz), 7.34-7.39 (m, 2H), 6.00 (dd, 1H, J=9.6, 4.0 Hz), 5.41 (d, 1H, J=46.8 Hz), 5.25 (dd, 1H, J=16.8, 3.6 Hz), 4.84 (d, 1H, J=14.0 Hz), 4.69 (d, 1H, J=14.0 Hz), 4.51-5.08 (m, 1H), 4.38 (dd, 1H, J=10.4, 5.2 Hz), 4.19 (dd, 1H, J=10.4, 6.4 Hz), 3.81-4.15 (m, 2H), 3.69 (d, 1H, J=11.2 Hz), 2.97-3.31 (m, 6H), 2.94 (s, 3H), 2.77-2.89 (m, 1H), 2.64-2.74 (m, 1H), 2.49 (s, 3H), 2.25-2.34 (m, 1H), 2.01-2.11 (m, 1H), 1.68-1.89 (m, 4H).

13-2: LC-MS (ESI): m/z=585.0 [M+1]⁺: ¹H NMR (400 MHz, CDCl₃): δ 7.82-7.84 (m, 2H), 7.75 (dd, 1H, J=7.2, 2.0 Hz), 7.50 (t, 1H, J=7.6 Hz), 7.33-7.38 (m, 2H), 6.00 (dd, 1H, J=10.4, 3.2 Hz), 5.48 (d, 1H, J=47.2 Hz), 5.25 (dd, 1H, J=16.8, 3.6 Hz), 4.97 (d, 1H, J=13.6 Hz), 4.76 (d, 1H, J=13.6 Hz), 4.52-5.05 (m, 1H), 4.41 (dd, 1H, J=10.8, 4.8 Hz), 4.21 (dd, 1H, J=10.8, 6.0 Hz), 3.98 (d, 1H, J=14.8 Hz), 3.91 (d, 1H, J=12.8 Hz), 3.37-3.78 (m, 2H), 2.97-3.31 (m, 4H), 2.91 (s, 3H), 2.69-2.84 (m, 3H), 2.54 (s, 3H), 2.33-2.40 (m, 1H), 2.03-2.14 (m, 1H), 1.76-1.93 (m, 4H).

### Example 14 Synthetic Route of Compound 14

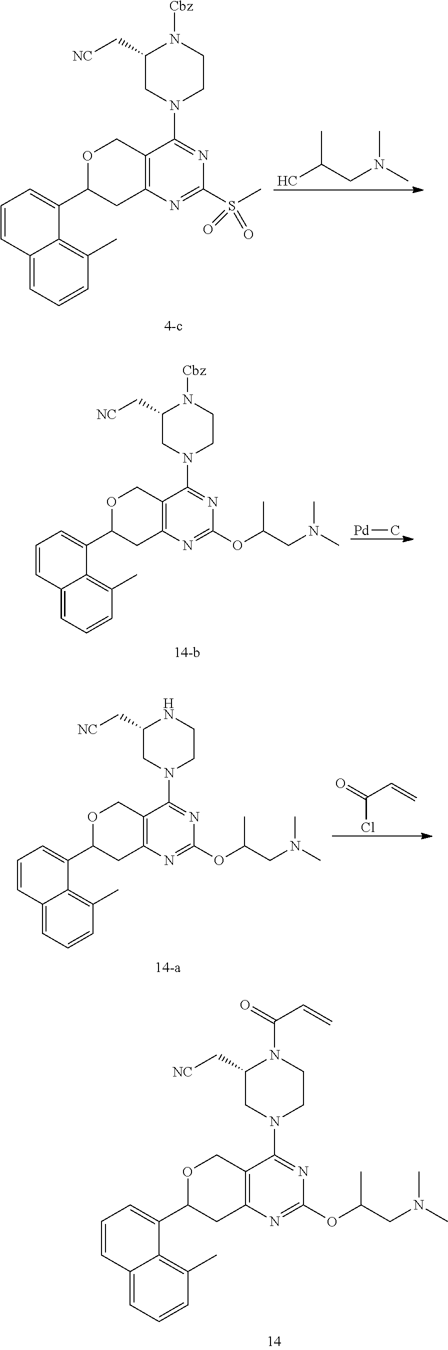

### Synthesis of Compound 14-b

Compound 4-c (100 mg, 0.164 mmol) was dissolved in toluene (10 mL); in an ice bath. N,N-dimethyl isopropanolamine (33.7 mg, 0.327 mmol) and sodium tert-butoxide (31.4 mg, 0.327 mmol) were added: under nitrogen atmosphere, the mixture was stirred at this temperature for 30 minutes, and TLC monitoring indicated that the reaction was incomplete; N,N-dimethyl isopropanolamine (33.7 mg, 0.327 mmol) and sodium tert-butoxide (31.4 mg, 0.327 mmol) were then supplemented: the mixture was continuously stirred for 30 minutes and TLC monitoring indicated that the reaction was complete; the product was concentrated and subjected to column (biotage, 12 g, silica gel, UV254, MeOH: DCM=0 to 8%) to give 14-b (82 mg, 79%) as a white solid. LC-MS (ESI): m/z=635.3 [M+H]⁺.

### Synthesis of Compound 14-a

14-b (82 mg, 0.129 mmol) was dissolved in methanol (10 mL) and ethyl acetate (10 mL), added 10% palladium-carbon (80 mg), replaced three times with hydrogen and stirred at room temperature for 2 hours; TLC monitoring indicated that the reaction was complete, the reaction was filtered and concentrated to give 14-a (53 mg, 82%) as a white solid. LC-MS (ESI): m/z=501.3 [M+H]⁺.

### Synthesis of Compound 14

14-a (53 mg, 0.106 mmol) was dissolved in dichloromethane (20 mL), and acryloyl chloride (14.3 mg, 0.159 mmol) and DIPEA (41 mg, 0.318 mmol) were successively added; under nitrogen atmosphere, the mixture was stirred at room temperature for 1 hour, and LCMS monitoring indicated that the reaction was complete; the reaction was then quenched with a saturated sodium bicarbonate solution, extracted with dichloromethane (30 mL×3), dried, concentrated and subjected to column (biotage, 25 g, silica gel, UV254. MeOH:DCM=0 to 10%) to give 14 (44 mg, 75%) as a white solid. LC-MS (ESI): m/z=555.3 [M+H]⁺; ¹H NMR (400 MHz, CDCL₃): δ 7.74-7.84 (m, 3H), 7.44-7.52 (m, 1H), 7.32-7.39 (m, 2H), 6.51-6.63 (m, 1H), 6.35-6.42 (m, 1H), 5.96-6.02 (m, 1H), 5.79-5.85 (m, 1H), 5.41-5.52 (m, 1H), 4.94-5.03 (m, 1H), 4.68-4.90 (m, 1H), 3.84-4.02 (m, 1H), 3.48-3.75 (m, 1H), 3.19-3.30 (m, 1H), 2.97-3.17 (m, 2H), 2.93 (d, 3H, J=12.4 Hz), 2.68-2.87 (m, 2H), 2.56-2.65 (m, 1H), 2.42 (s, 6H), 1.96-2.33 (m, 5H), 1.33-1.38 (m, 3H).

### Example 15 Synthetic Route of Compound 15

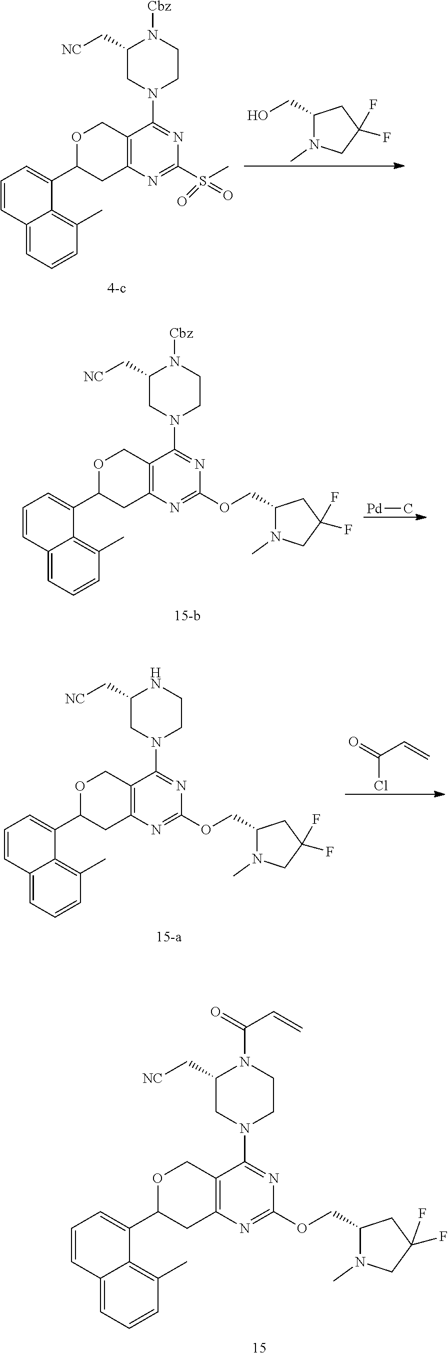

### Synthesis of Compound 15-b

4-c (100 mg, 0.0.164 mmol) was dissolved in toluene (10 mL); in an ice bath. S-4,4-difluoro-1-methylpyrrolidin-2-methanol (49.4 mg, 0.327 mmol) and sodium tert-butoxide (31.4 mg, 0.327 mmol) were added; under nitrogen atmosphere, the mixture was stirred at this temperature for 30 minutes; LCMS monitoring indicated that the reaction was complete; the reaction was then quenched with water, extracted with ethyl acetate (50 mL×2), dried, concentrated and subjected to column (biotage, 40 g, silica gel, UV254. EA:PE=0 to 100%) to give 15-b (97 mg, 87%) as a colorless gum. LC-MS (ESI): m/z=683.3 [M+H]⁺.

### Synthesis of Compound 15-a

15-b (97 mg, 0.142 mmol) was dissolved in ethyl acetate (10 mL) and methanol (10 mL), added 10% palladium-carbon (97 mg), replaced three times with hydrogen and stirred at room temperature for 2 hours; TLC monitoring indicated that the reaction was complete; the product was filtered through diatomite and subjected to rotary evaporation to give 15-a (70 mg, 90%) as a solid, which was used directly in the next reaction. LC-MS (ESI): m/z=549.0 [M+H]⁺.

### Synthesis of Compound 15

15-a (70 mg, 0.128 mmol) was dissolved in dichloromethane (15 mL), and acryloyl chloride (17.2 mg, 0.192 mmol) and DIPEA (82.4 mg, 0.639 mmol) were successively added; under nitrogen atmosphere, the mixture was stirred at room temperature for 1 hour; LCMS monitoring indicated that the reaction was complete; the reaction was then quenched with a saturated sodium bicarbonate solution, extracted with dichloromethane (30 mL×3), dried, concentrated and subjected to column (biotage, 12 g, silica gel, UV254, EA:PE=0 to 100%) to give 15 (51 mg, 66%) as a white solid. LC-MS (ESI): m/z=603.3 [M+H]⁺; ¹H NMR (400 MHz, CDCl₃): δ 7.73-7.86 (m, 3H), 7.44-7.52 (m, 1H), 7.32-7.40 (m, 2H), 6.51-6.62 (m, 1H), 6.36-6.43 (m, 1H), 5.96-6.03 (m, 1H), 5.83 (d, 1H), 4.92-5.07 (m, 1H), 4.67-4.89 (m, 2H), 4.41-4.48 (m, 1H), 4.22-4.32 (m, 1H), 3.85-4.03 (m, 2H), 3.64-3.75 (m, 1H), 3.36-3.57 (m, 2H), 2.89-3.18 (m, 8H), 2.61-2.85 (m, 3H), 2.43-2.59 (m, 4H), 2.18-2.39 (m, 1H).

### Example 16 Synthetic Route of Compound 16

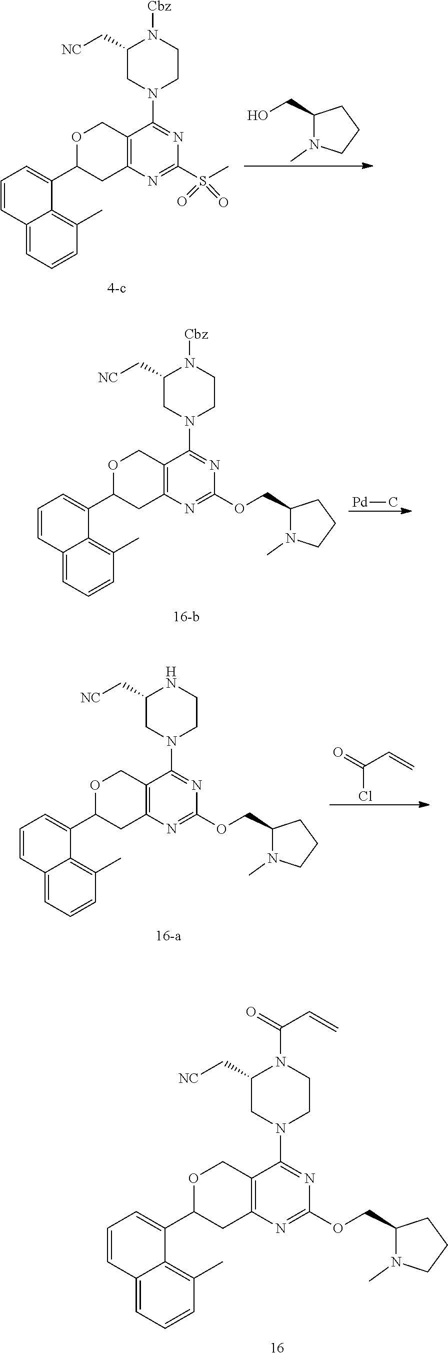

### Synthesis of Compound 16-b

4-c (100 mg, 0.164 mmol) was dissolved in toluene (10 mL); in an ice bath, N-methyl-D-prolinol (37.6 mg, 0.327 mmol) and sodium tert-butoxide (31.4 mg, 0.327 mmol) were added; under nitrogen atmosphere, the mixture was stirred at this temperature for 30 minutes; TLC monitoring indicated that the reaction was complete; the reaction was then quenched with water, extracted with ethyl acetate (50 mL×2), dried, concentrated and subjected to column (ISCO, 12 g, silica gel, UV254. MeOH:DCM=0 to 7%) to give 16-b (92 mg, 87%) as a white solid. LC-MS (ESI): m/z=647.3 [M+H]⁺.

### Synthesis of Compound 16-a

16-b (92 mg, 0.142 mmol) was dissolved in ethyl acetate (10 mL) and methanol (10 mL), added 10% palladium-carbon (90 mg), replaced three times with hydrogen and stirred at room temperature for 2 hours; LCMS monitoring indicated that the reaction was complete; the product was filtered through diatomite and subjected to rotary evaporation to give 16-a (62 mg, 85%) as a white solid, which was used directly in the next reaction. LC-MS (ESI); m/z=513.3 [M+H]⁺.

### Synthesis of Compound 16

16-a (62 mg, 0.121 mmol) was dissolved in dichloromethane (20 mL), and acryloyl chloride (16.3 mg, 0.182 mmol) and DIPEA (46.9 mg, 0.363 mmol) were successively added; under nitrogen atmosphere, the mixture was stirred at room temperature for 1 hour; LCMS monitoring indicated that the reaction was complete; the reaction was then quenched with a saturated sodium bicarbonate solution, extracted with dichloromethane (40 mL×4), dried, concentrated and subjected to column (biotage, 25 g, silica gel, UV254. MeOH:DCM=0 to 15%) to give 16 (60 mg, 87%) as a pale brown solid. LC-MS (ESI): m/z=567.3 [M+H]⁺; ¹H NMR (400 MHz, CDCl₃): δ 7.74-7.84 (m, 3H), 7.44-7.52 (m, 1H), 7.32-7.39 (m, 2H), 6.51-6.53 (m, 1H), 6.24-6.41 (m, 1H), 5.97-6.16 (m, 1H), 5.68-5.84 (m, 1H), 4.95-5.12 (m, 1H), 4.68-4.88 (m, 3H), 4.30-4.39 (m, 1H), 3.36-3.75 (m, 3H), 3.01-3.31 (m, 5H), 2.93 (d, 3H, J=15.6 Hz), 2.61-2.83 (m, 4H), 2.51-2.61 (m, 1H), 1.84-2.31 (m, 7H).

### Example 17 Synthetic Route of Compound 17

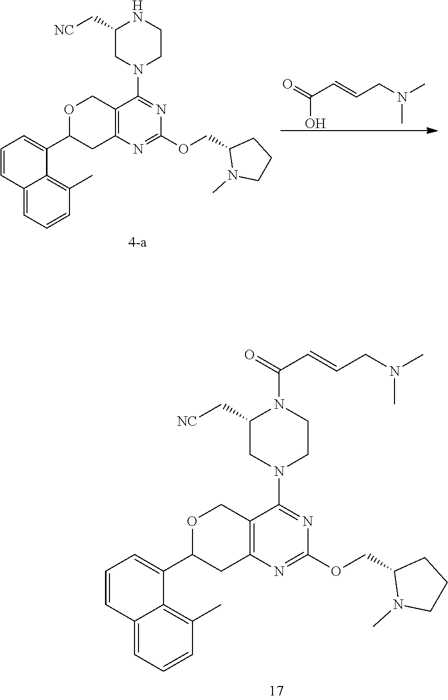

To a solution of 4-a (50 mg, 0.1 mmol) and HATU (74.2 mg, 0.2 mmol) in DMF (5 mL) were respectively added DIPEA (0.05 mL, 0.3 mmol) and (2.E)-4-(dimethylamino)-2-butenoic acid (18.9 mg, 0.015 mmol). The reaction mixture was heated to 60° C., and stirred overnight. After completion of the reaction, the solid was precipitated out by slowly adding water, collected by filtration and washed with water. After drying, a crude product was obtained and purified by silica gel column chromatography (DCM/MeOH 4/1) to give product 17 (17 mg, 28%) as a light brown solid. LC-MS (ESI); m/z=624.3 [M+H]⁻: ¹H NMR (400 MHz, CDCl₃): δ 7.74-7.84 (m, 3H), 7.46-7.52 (m, 1H), 7.34-7.37 (m, 2H), 6.89-7.00 (m, 1H), 6.38-6.63 (m, 1H), 5.96-6.00 (m, 1H), 4.89-5.12 (m, 1H), 4.48-4.89 (m, 3H), 4.21-4.35 (m, 1H), 3.86-4.08 (m, 1H), 3.56-3.78 (m, 1H), 3.30-3.55 (m, 2H), 2.85-3.29 (m, 8H), 2.92 (d, 3H, J=9.6 Hz), 2.66 (d, 3H, J=8.8 Hz), 2.41-2.57 (m, 1H), 2.32 (s, 6H), 2.04-2.22 (m, 2H), 1.76-2.03 (m, 4H).

### Example 18 Synthetic Route of Compound 18

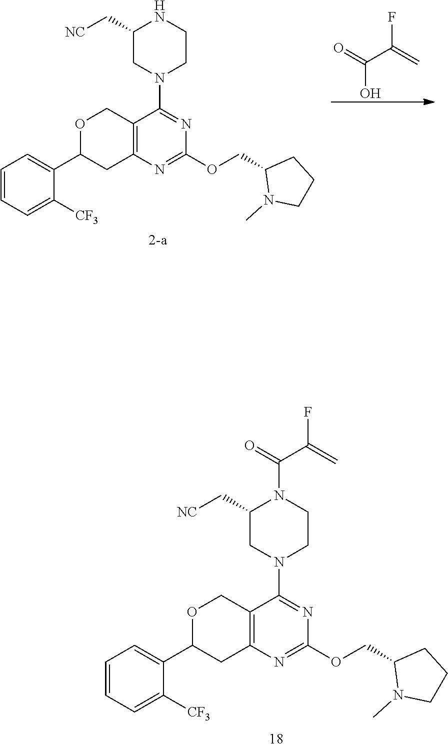

### Synthesis of Compound 18

At room temperature, compound 2-a (0.3 g, 0.58 mmol) was dissolved in DMF (10 mL), and DIPEA (0.15 g, 1.16 mmol), 2-fluoroacrylic acid (0.105 g, 1.16 mmol) and HATU (0.44 g, 1.16 mmol) were successively added. Under nitrogen atmosphere, the mixture was stirred at room temperature for 20 hours, and then the solid was precipitated out by adding water (100 mL), filtered and dried in vacuum to give compound 18 (300 mg, 88%) as a white solid. LC-MS (ESI): m/z=589.2 [M+1]⁺: ¹H NMR (500 MHz, CD₃OD): δ 7.82-7.87 (m, 1H), 7.66-7.78 (m, 2H), 7.54 (t, 1H, J=9.5 Hz), 5.33-5.41 (m, 1H), 5.27-5.32 (m, 1H), 5.18-5.23 (m, 1H), 5.03 (d, 1H, J=20.0 Hz), 4.92-4.98 (m, 1H), 4.75-4.88 (m, 2H), 4.52 (t, 1H, J=8.5 Hz), 4.20 (d, 1H, J=9.5 Hz), 3.80-4.05 (m, 2H), 3.60-3.77 (m, 1H), 3.35-3.59 (m, 2H), 3.05-3.32 (m, 3H), 3.07 (d, 3H, J=9.0 Hz), 2.83-3.03 (m, 3H), 2.30-2.43 (m, 1H), 2.15-2.26 (m, 1H), 1.98-2.15 (m, 2H), 1.28-1.41 (m, 1H).

### Synthesis of Compounds 18-1 and 18-2

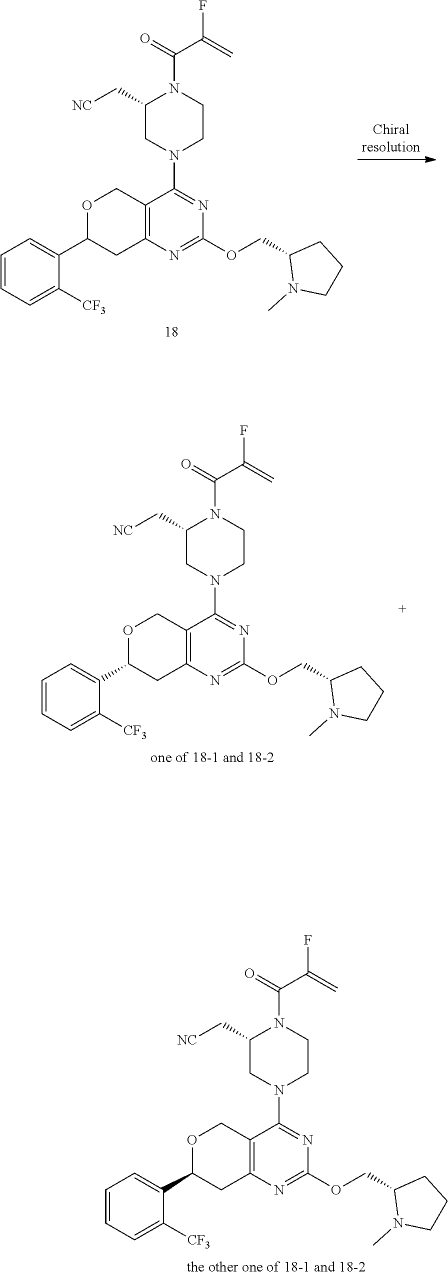 [SMILES: C=C(F)C(=O)N1CCN(c2nc(OC[C@@H]3CCCN3C)nc3c2COC(c2ccccc2C(F)(F)F)C3)C[C@@H]1CC#N ; C=C(F)C(=O)N1CCN(c2nc(OC[C@@H]3CCCN3C)nc3c2CO[C@@H](c2ccccc2C(F)(F)F)C3)C[C@@H]1CC#N ; C=C(F)C(=O)N1CCN(c2nc(OC[C@@H]3CCCN3C)nc3c2CO[C@H](c2ccccc2C(F)(F)F)C3)C[C@@H]1CC#N]

Compound 18 (280 mg, 0.48 mmol) was purified by chiral resolution to give compound 18-1 (96 mg, 34%) and compound 18-2 (68 mg, 24%).

| Chiral analysis conditions | Chiral preparation conditions |
|---|---|
| equipment: SFC Method Station | instrument: SFC-150 (Thar, |
| (Thar, Waters) | Waters) |
| chromatographic column: OD-H | chromatographic column: OD |
| 4.6 * 100 mm, 5 μm (Daicel) | 20 * 250 mm, 10 μm (Daicel) |
| column temperature: 40° C. | column temperature: 35° C. |
| mobile phase: CO2/MeOH (1% | mobile phase: CO2/MeOH |
| TEA) = 65/35 | (0.1% TEA) = 50/50 |
| flow rate: 4.0 ml/min | flow rate: 120 g/min |
| wavelength: 254 nm | back pressure: 100 bar |
| back pressure: 120 bar | detection wavelength: 214 nm |
| | cycling time: 4.5 min |
| 18-1: retention time: 0.78 min, | |
| d.e. % = 100%; | |
| 18-2: retention time: 2.42 min, | |
| d.e. % = 99.2% | |

18-1: LC-MS (ESI): m/z=589.2 [M+1]⁺: ¹H NMR (500 MHz, CD₃OD): δ 7.84 (d, 1H, J=10.0 Hz), 7.68-7.78 (m, 2H), 7.54 (t, 1H, J=10.0 Hz), 5.34 (d, 1H, 3=55.5 Hz), 5.33 (dd, 1H, J₁=4.5 Hz, J₂=21.0 Hz), 5.20 (dd, 1H, J₁=4.5 Hz, J₂=13.5 Hz), 5.03 (d, 1H, J=17.5 Hz), 4.85 (d, 1H, J=17.0 Hz), 4.73 (dd, 1H, J₁=4.5 Hz, J₂=16.0 Hz), 4.51 (dd, 1H, J₁=8.5 Hz, J₂=15.5 Hz), 4.47 (d, 1H, J=15.0 Hz), 3.91 (d, 1H, J=12.5 Hz), 3.70-3.86 (m, 1H), 3.60-3.70 (m, 1H), 3.30-3.42 (m, 2H), 3.10-3.28 (m, 3H), 3.01 (s, 3H), 2.96-3.07 (m, 1H), 2.80-2.94 (m, 1H), 2.30-2.43 (m, 1H), 1.97-2.21 (m, 3H).

18-2: LC-MS (ESI): z=589.2 [M+1]⁻; ¹H NMR (500 MHz, CDCl₃): δ 7.75 (d, 1H, J=10.0 Hz), 7.60-7.70 (m, 2H), 7.45 (t, 1H, J=9.5 Hz), 5.42 (d, 1H, J=59.5 Hz), 5.25 (dd, 1H, J₁=4.0 Hz, J₂=21.0 Hz), 5.17 (dd, 1H, J₁=4.0 Hz, J₂=14.0 Hz), 4.88 (d, 1H, J=17.0 Hz), 4.77 (d, 1H, J=16.5 Hz), 4.39 (dd, 1H, J₁=6.5 Hz, J₂=13.0 Hz), 4.16 (dd, 1H, J₁=9.0 Hz, J₂=13.0 Hz), 3.96 (d, 1H, J=17.5 Hz), 3.74 (d, 1H, J=16.5 Hz), 3.48 (d, 1H, J=14.0 Hz), 2.97-3.13 (m, 3H), 2.60-2.96 (m, 4H), 2.49 (s, 3H), 2.26-2.34 (m, 1H), 1.97-2.10 (m, 1H), 1.60-1.88 (m, 6H).

### Example 19 Synthesis of Compounds 19-1 and 19-2

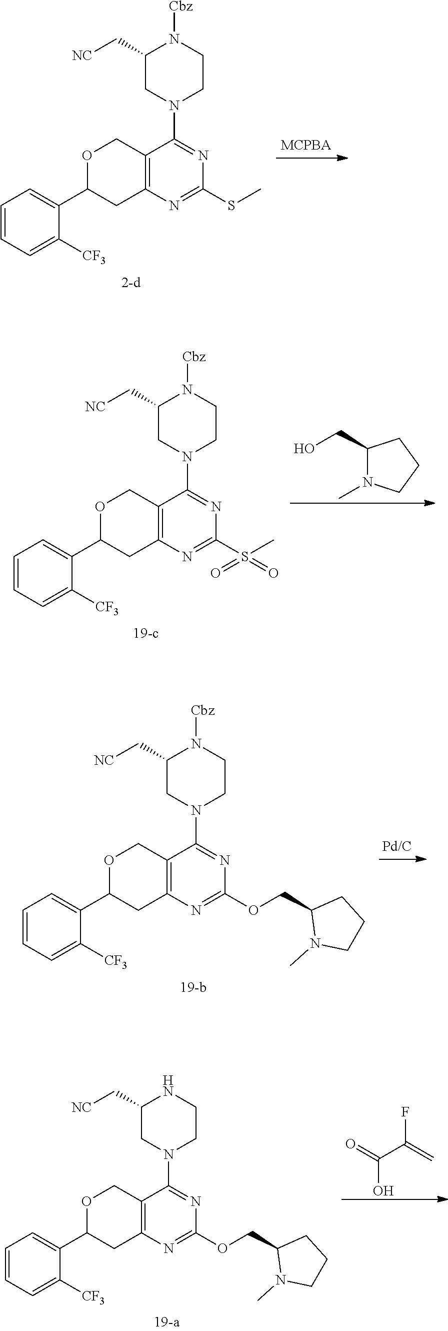

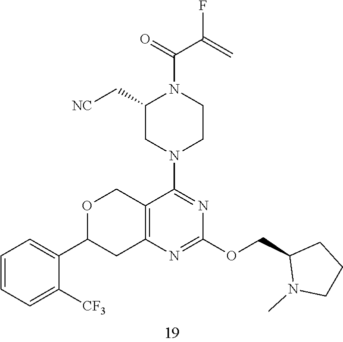 [SMILES: C=C(F)C(=O)N1CCN(c2nc(OC[C@H]3CCCN3C)nc3c2COC(c2ccccc2C(F)(F)F)C3)C[C@@H]1CC#N]

### Synthesis of Compound 19-c

2-d (500 mg, 0.858 mmol) was dissolved in ethyl acetate (25 mL), added MCPBA (434 mg, 2.145 mmol) and stirred at room temperature for 2 hours: LCMS monitoring indicated that the reaction was complete; the reaction was then quenched with a saturated sodium bicarbonate solution, extracted with ethyl acetate (30 mL×3), dried and concentrated to give 19-c (527 mg, 100%) as a pale yellow solid, which was used directly in the next reaction without purification. LC-MS (ESI): m/z=616.2 [M+H]⁺.

### Synthesis of Compound 19-b

19-c (527 mg, 0.857 mmol) was dissolved in toluene (25 mL); in all ice bath. N-methyl-D-prolinol (197 mg, 1.71 mmol) and sodium tert-butoxide (165 mg, 1.72 mmol) were added; under nitrogen atmosphere, the mixture was stirred at this temperature for 30 minutes: TLC monitoring indicated that the reaction was complete; the reaction was then quenched with water, extracted with ethyl acetate (50 mL×2), dried, concentrated and subjected to column (biotage, 40 g, silica gel, UV254. MeOH:DCM=0 to 10%) to give 19-b (452 mg, 81%) as a white solid. LC-MS (ESI): m/z=651.2 [M+H]⁺.

### Synthesis of Compound 19-a

19-b (452 me, 0.695 mmol) was dissolved in 7 N methanolic ammonia solution (50 nit) and added 10% palladium-carbon (250 mg) in an dry ice-acetone bath; the mixture was replaced three tunes with hydrogen and stirred at room temperature for 1 hour; TLC monitoring indicated that the reaction was complete; the product was filtered through diatomite and subjected to rotary evaporation to give 19-a (359 mg, 100%) as a white solid, which was used directly in the next reaction. LC-MS (ESI): m/z=517.2 [M+H]⁺.

### Synthesis of Compound 19

19-a (359 tug, 0.696 mmol) was dissolved in DMF (15 mL), and 2-fluoroacrylic acid (125 mg, 1.39 mmol). HATU (529 mg, 1.39 mmol) and DIPEA (449 mg, 3.48 mmol) were successively added; under nitrogen atmosphere, the mixture was stirred at room temperature for 1.5 hours; LCMS monitoring indicated that the reaction was complete; the reaction was then quenched with a saturated sodium bicarbonate solution and extracted with ethyl acetate (40 mL×3); the organic phase was washed with saturated brine (50 mL×3), dried, concentrated and subjected to column (biotage, 40 g, silica gel, UV254. MeOH:DCM=0 to 10%) to give 19 (346 mg, 85%) as a pale brown solid. LC-MS (ESI): m/z=589.0 [M+H]⁺.

### Synthesis of Compounds 19-1 and 19-2

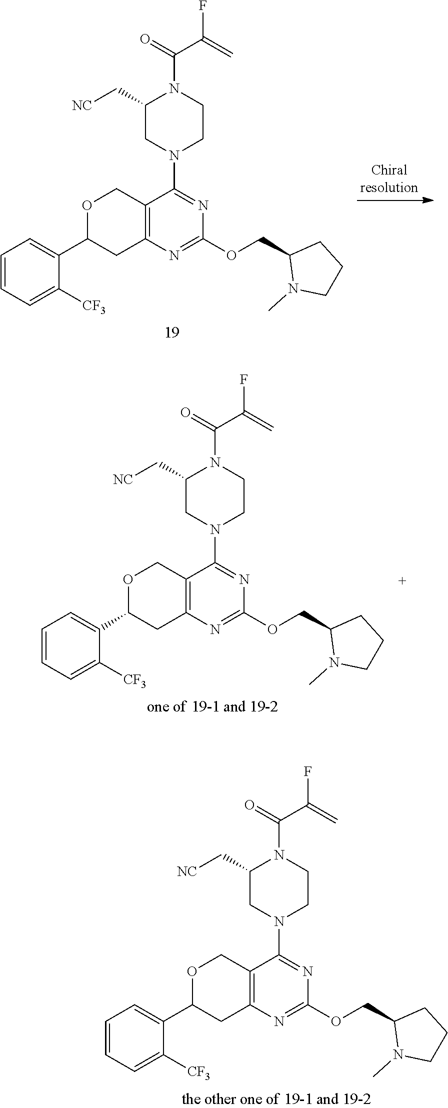 [SMILES: C=C(F)C(=O)N1CCN(c2nc(OC[C@H]3CCCN3C)nc3c2COC(c2ccccc2C(F)(F)F)C3)C[C@@H]1CC#N ; C=C(F)C(=O)N1CCN(c2nc(OC[C@H]3CCCN3C)nc3c2CO[C@@H](c2ccccc2C(F)(F)F)C3)C[C@@H]1CC#N ; C=C(F)C(=O)N1CCN(c2nc(OC[C@H]3CCCN3C)nc3c2COC(c2ccccc2C(F)(F)F)C3)C[C@@H]1CC#N]

19 (346 mg, 0.588 mmol) was subjected to resolution using chiral preparation and then subjected to rotary evaporation and lyophilized to give 19-1 (136 mg, 39%) and 19-2 (98 mg, 28%) as a white solid.

| Chiral analysis conditions | Chiral preparation conditions |
|---|---|
| equipment: SFC Method Station | instrument: SFC-150 (Thar, |
| (Thar, Waters) | Waters) |
| chromatographic column: OD-H | chromatographic column: OD 20 * |
| 4.6 * 100 mm, 5 μm (Daicel) | 250 mm, 10 μm (Daicel) |
| column temperature: 40° C. | column temperature: 35° C. |
| mobile phase: CO₂/MeOH (0.1% | mobile phase: CO₂/Methanol (0.1% |
| TEA) = 65/35 | TEA) = 40/60 |
| flow rate: 4.0 ml/min | flow rate: 120 g/min |
| wavelength: 254 nm | back pressure: 100 bar |
| back pressure: 120 bar | detection wavelength: 214 nm |
| | cycling time: 3.0 min |
| 19-1: retention time: 0.79 min; | |
| d.e. % = 100%; | |
| 19-2: retention time: 2.29 min; | |
| d.e. % = 100%. | |

19-1: LC-MS (ESI): m/z=589.0 [M+H]⁺: ¹H NMR (400 MHz, CDCl₃): δ 7.74 (d, 1H, J=8.0 Hz), 7.67 (d, 1H, J=8.0 Hz), 7.63 (t, 1H, J=7.6 Hz), 7.44 (t, 1H, J=7.6 Hz), 5.38 (d, 1H, J=48.0 Hz), 5.24 (dd, 1H, J=3.6, 16.8 Hz), 5.19 (dd, 1H, J=4.0, 11.2 Hz), 4.89 (d, 1H, J=13.6 Hz), 4.72 (d, 1H, J=13.6 Hz), 4.51-4.65 (m, 3H), 3.74-3.82 (m, 1H), 3.65-3.72 (m, 1H), 3.56-3.64 (m, 1H), 3.26-3.43 (m, 2H), 2.99-3.17 (m, 5H), 2.83-2.95 (m, 2H), 2.68-2.79 (m, 2H), 2.08-2.37 (m, 4H).

19-2: LC-MS (ESI): m/z=589.0 [M+H]⁺: ¹H NMR (400 MHz, CDCl₃): δ 7.76 (d, 1H, J=7.6 Hz), 7.67 (d, 1H, J=8.0 Hz), 7.63 (t, 1H, J=7.6 Hz), 7.44 (t, 1H, J=8.0 Hz), 5.41 (d, 1H, J=47.6 Hz), 5.25 (dd, 1H, 3=3.6, 16.8 Hz), 5.17 (dd, 1H, J=3.2, 11.2 Hz), 4.88 (d, 1H, J=14.0 Hz), 4.77 (d, 1H, J=13.6 Hz), 4.41 (dd, 1H, J=5.2, 10.8 Hz), 4.18 (dd, 1H, =6.4, 10.4 Hz), 3.98 (d, 1H, J=14.4 Hz), 3.70-3.78 (m, 1H), 3.44-3.54 (m, 1H), 2.99-3.17 (m, 3H), 2.67-2.97 (m, 4H), 2.50 (s, 3H), 2.25-2.37 (m, 1H), 2.02-2.12 (m, 1H), 1.68-1.89 (m, 5H).

### Example 20 Synthetic Route of Compound 20

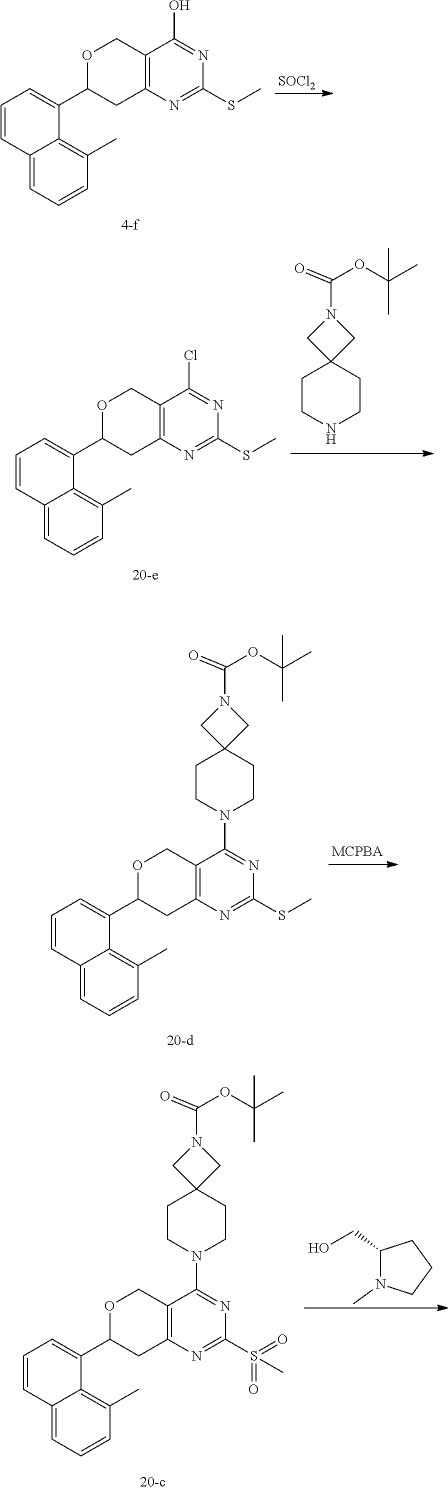

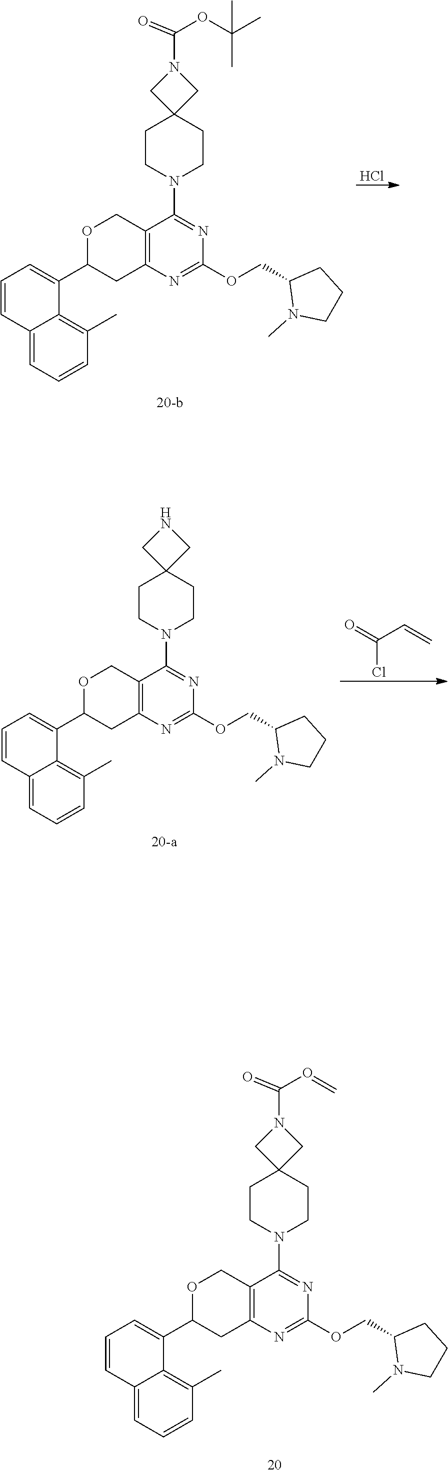

### Synthesis of Compound 20-e

Compound 4-f (1.7 g, 5.0 mmol) was dissolved in DMF (20 mL) and DCM (10 mL); at 0° C. under nitrogen atmosphere, thionyl chloride (2.5 mL, 34.4 mmol) was added dropwise: and the mixture was stirred at 0° C. for 3 hours. The reaction was quenched by adding ice water (80 mL, 120 mmol), extracted with dichloromethane (80 mL*2) and concentrated. The crude product was separated and purified through a flash column chromatography (petroleum ether/ethyl acetate=25/1) to give compound 20-e (1.1 g, 62%) as a pale yellow solid. LC-MS (ESI): m/z=357.1 [M+1]⁺.

### Synthesis of Compound 20-d

At room temperature, compound 20-e (0.25 g, 0.70 mmol) was dissolved in DMF (20 mL), and DIPEA (0.135 g, 1.05 mmol), 2-tert-butoxycarbonyl-2,7-diazaspiro[3.5]nonane (0.24 g, 1.05 mmol) were successively added. The mixture was stirred at 100° C. for 2 hours and then quenched by adding water (100 mL); the solid was precipitated out by filtration and dried to give compound 20-d (0.29 g, 76%) as a grey solid. LC-MS (ESI): m/z=547.3 [M+1]⁺.

### Synthesis of Compound 20-c

Compound 20-d (0.29 g, 0.53 mmol) was dissolved in ethyl acetate (20 mL), and MCPBA (0.28 g, 1.36 mmol) was added at room temperature. The mixture was stirred at room temperature for 1 hour and then quenched by adding saturated sodium bicarbonate solution (500 mL) and extracted with ethyl acetate (50 mL*2); the organic phase was concentrated, and the crude product was separated and purified through a flash column chromatography (DCM/MeOH=20/1) to give compound 20-c (0.31 g, 98%) as a white solid. LC-MS (ESI): m/z=579.3 [M+1]⁺.

### Synthesis of Compound 20-b

Under ice-water bath cooling, to compound 20-c (0.31 g, 0.536 mmol) in toluene (15 mL). N-methyl-L-prolinol (0.111 g, 0.97 mmol) and t-BuONa (0.103 g, 1.07 mmol) were successively added. Under nitrogen atmosphere, the mixture was stirred in an ice-water bath for 0.5 hours and then quenched by adding water (10 mL) and extracted with ethyl acetate (30 mL*2); the organic phase was concentrated and the crude product was separated and purified through a flash column chromatography (MeOH/DCM=1.19) to give compound 20-b (0.28 g, 85%) as a white solid. LC-MS (ESI): m/z=614.4 [M+1]⁺.

### Synthesis of Compound 20-a

Compound 20-b (0.28 g, 0.46 mmol) was added to a mixture of dichloromethane (15 mL) and trifluoro acetic acid (15 mL), stirred for 2 hours under nitrogen atmosphere and concentrated to give compound 20-a (0.26 g). LC-MS (ESI): m/z=514.3 [M+1]⁺.

### Synthesis of Compound 20

At room temperature, compound 20-a (0.26 g, 0.51 mmol) was dissolved in DCM (20 mL) and successively added DIPEA (0.15 g, 1.16 mmol) and acryloyl chloride (0.068 g, 0.76 mmol): under nitrogen atmosphere, the mixture was stirred at room temperature for 2 hours and then extracted by adding water (10 mL) and dichloromethane (30 mL*2); the organic phase was concentrated: the crude product was separated and purified through a flash column chromatography (MeOH/DCM=1/20) to give compound 20 (0.06 g, 23% for two reaction steps) as a white solid. LC-MS (ESI): m/z=568.3 [M+1]⁺: ¹H NMR (500 MHz, CD₃OD): δ 7.85 (d, 1H, J=10.0 Hz), 7.83 (d, 1H, J=9.5 Hz), 7.60-7.80 (m, 1H), 7.48 (t, 1H, J=9.5 Hz), 7.33-7.41 (m, 2H), 6.20-6.43 (m, 2H), 6.06 (dd, 1H, J₁=5.0 Hz, J₂=12.5 Hz), 5.76 (dd, 1H, J₁=2.0 Hz, J₂=12.5 Hz), 5.01 (d, 1H, J=17.5 Hz), 4.68 (d, 1H, J=17.5 Hz), 4.41-4.59 (m, 2H), 4.08 (dd, 2H, J₁=10.5 Hz, J₂=16.0 Hz), 3.84 (dd, 2H, J₁=13.0 Hz, J₂=16.0 Hz), 3.46-3.60 (m, 2H), 3.34-3.45 (m, 4H), 3.10-3.22 (m, 1H), 2.94 (s, 3H), 2.80-2.92 (m, 1H), 2.82 (s, 3H), 2.20-2.31 (m, 1H), 1.79-2.10 (m, 8H).

### Example 21 Synthetic Route of Compound 32

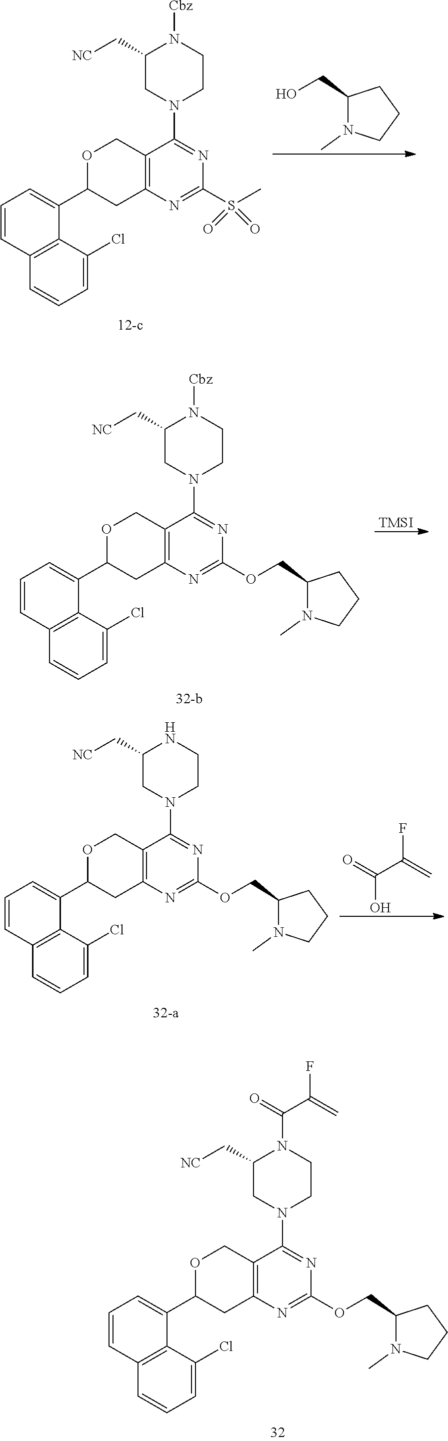

### Synthesis of Compound 32-b

12-c (527 mg, 0.333 mmol) was dissolved in toluene (15 mL); in an ice bath, N-methyl-D-prolinol (76.5 mg, 0.666 mmol) and sodium tert-butoxide (64 mg, 0.667 mmol) were added: under nitrogen atmosphere, the mixture was stirred at this temperature for 30 minutes: TLC monitoring indicated that the reaction was complete; the reaction was then directly mixed with silica gel and subjected to column (biotage, 25 g, silica gel, UV254. MeOH:DCM=0 to 10%) to give 32-b (202 mg, 91%) as a white solid. LC-MS (ESI): m/z=667.3 [M+H]⁺.

### Synthesis of Compound 32-a

32-b (202 mg, 0.303 mmol) was dissolved in acetonitrile (20 mL) and added trimethyliodosilane (200 uL, 1.4 mmol): under nitrogen atmosphere, at 30° C., the mixture was stirred for 2 hours: LCMS monitoring indicated that the reaction was incomplete, and trimethyliodosilane (827 mg, 4.13 mmol) was then supplemented: the mixture was continuously stirred for 2 hours; LCMS monitoring indicated that the reaction was complete, and then 1 mL, of triethylamine was added; the mixture was subjected to rotary evaporation to give 32-a (crude product) as a dark solid, which was used directly in the next reaction without further purification. LC-MS (ESI): m/z=533.0 [M+H]⁺.

### Synthesis of Compound 32

32-a (crude product) was dissolved in DMF (10 mL), and 2-fluoroacrylic acid (54.6 mg, 0.607 mmol). HATU (231 mg, 0.608 mmol) and DIPEA (196 mg, 1.52 mmol) were successively added: under nitrogen atmosphere, the mixture was stirred at room temperature for 1.5 overnight: LCMS monitoring indicated that the reaction was complete; the reaction was then quenched with a saturated sodium bicarbonate solution and extracted with ethyl acetate (30 mL×3); the organic phase was washed with saturated brine (50 mL×3), dried, concentrated and subjected to column (biotage, 40 g, silica gel, UV254. MeOH: DCM=0 to 10%) to give 32 (151 mg, 82%). LC-MS (ESI): m/z=605.0 [M+H]⁺.

### Synthesis of Compounds 32-1 and 32-2

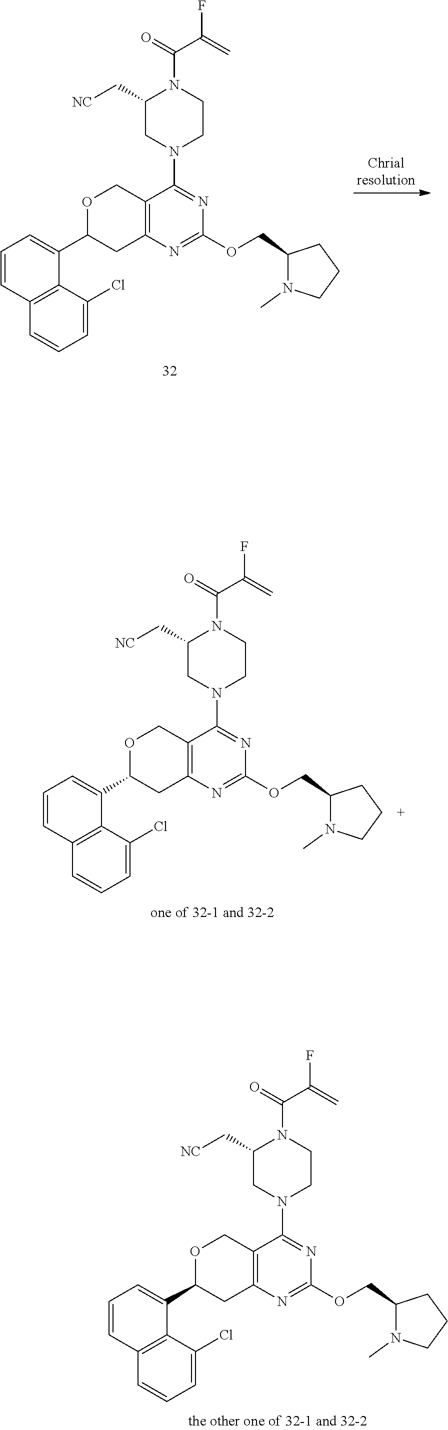 [SMILES: C=C(F)C(=O)N1CCN(c2nc(OC[C@H]3CCCN3C)nc3c2COC(c2cccc4cccc(Cl)c24)C3)C[C@@H]1CC#N ; C=C(F)C(=O)N1CCN(c2nc(OC[C@H]3CCCN3C)nc3c2CO[C@@H](c2cccc4cccc(Cl)c24)C3)C[C@@H]1CC#N ; C=C(F)C(=O)N1CCN(c2nc(OC[C@H]3CCCN3C)nc3c2CO[C@H](c2cccc4cccc(Cl)c24)C3)C[C@@H]1CC#N]

32 (151 mg, 0.25 mmol) was subjected to resolution using chiral preparation and then subjected to rotary evaporation and lyophilized to give 32-1 (46 mg, 30%) and 32-2 (51 mg, 34%) as a white solid.

| Chiral analysis conditions | Chiral preparation conditions |
|---|---|
| equipment: SFC Method Station | instrument: SFC-150 (Thar, |
| (Thar, Waters) | Waters) |
| chromatographic column: OJ-H | chromatographic column: OJ |
| 4.6 * 100 mm, 5 μm (Daicel) | 20 * 250 mm, 10 μm (Daicel) |
| column temperature: 40° C. | column temperature: 35° C. |
| mobile phase: CO₂/MeOH (0.1% | mobile phase: CO₂/Methanol |
| TEA) = 60/40 | (0.1% TEA) = 40/60 |
| flow rate: 4.0 ml/min | flow rate: 120 g/min |
| wavelength: 254 nm | back pressure: 100 bar |
| back pressure: 120 bar | detection wavelength: 214 nm |
| | cycling time: 5.0 min |
| 32-1: retention time: 1.45 min; | |
| d.e. % = 100%; | |
| 32-2: retention time: 2.81 min; | |
| d.e. % = 100%. | |

32-1: LC-MS (ESI): m/z=604.9 [M+H]⁺: ¹H NMR (400 MHz, CDCl₃): δ 7.96 (d, 1H, J=7.2 Hz), 7.83 (t, 2H, J=8.8 Hz), 7.60 (d, 1H, J=7.2 Hz), 7.55 (t, 1H, J=7.6 Hz), 7.37 (t, 1H, J=7.6 Hz), 6.48 (dd, 1H, J=2.8, 10.8 Hz), 5.42 (d, 1H, J=47.6 Hz), 5.26 (dd, 1H, J=2.8, 16.8 Hz), 4.97 (d, 1H, J=13.6 Hz), 4.84 (d, 1H, J=14.0 Hz), 4.38 (dd, 1H, J=4.8, 10.8 Hz), 4.18 (dd, 1H, J=6.8, 10.8 Hz), 3.98 (d, 1H, J=10.0 Hz), 3.77 (d, 1H, J=10.4 Hz), 3.58 (d, 1H, J=16.8 Hz), 3.18-3.31 (m, 2H), 3.02-3.13 (m, 2H), 2.84-2.94 (m, 2H), 2.63-2.72 (m, 1H), 2.48 (s, 3H), 2.23-2.33 (m, 1H), 1.96-2.10 (m, 1H), 1.69-1.90 (m, 6H).

32-2: LC-MS (ESI): m/z=605.0 [M+H]⁺: ¹H NMR (400 MHz, CDCl₃): δ 7.97 (d, 1H, J=6.8 Hz), 7.84 (t, 2H, J=9.2 Hz), 7.49-7.67 (m, 2H), 7.37 (t, 1H, J=8.4 Hz), 6.51 (d, 1H, J=10.8 Hz), 5.40 (d, 1H, J=50.0 Hz), 5.25 (d, 1H, J=15.6 Hz), 4.99 (d, 1H, J=14.0 Hz), 4.83 (d, 1H, J=13.6 Hz), 4.54-4.66 (m, 1H), 4.42-4.54 (m, 1H), 4.08-4.17 (m, 1H), 3.76-3.91 (m, 1H), 3.50-3.60 (m, 3H), 3.33-3.47 (m, 1H), 3.00-3.33 (m, 5H), 2.80-2.92 (m, 3H), 1.98-2.41 (m, 7H).

### Example 22 Synthetic Route of Compound 22

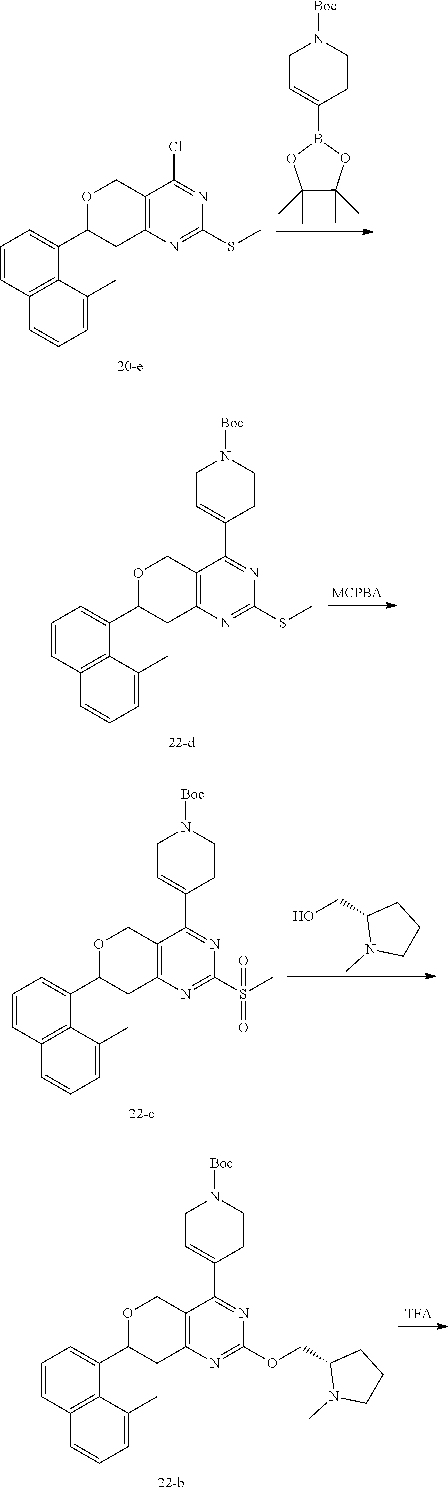

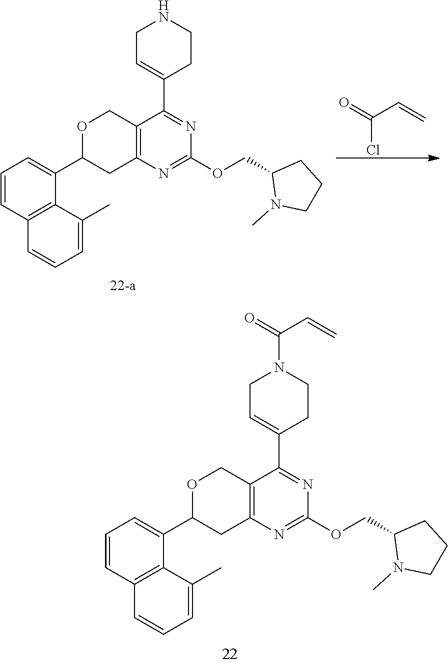

### Synthesis of Compound 22-d

20-e (100 mg, 0.281 mmol) and N-Boc-1,2,5,6-tetrahydropyridine-4-boronic acid pinacol ester (104 mg, 0.337 mmol) were dissolved in 1,4-dioxane (10 mL), and water (1 mL), potassium carbonate (116 mg, 0.84 mmol) and tetrakis(triphenylphosphine)palladium (32 mg, 0.028 mmol) were successively added; the mixture was replaced three times with nitrogen and stirred at 100° C. overnight; LCMS monitoring indicated that the reaction was complete; the reaction was directly subjected to rotary evaporation, mixed with silica gel and subjected to column (biotage, 25 g, silica gel, UV254, EA:PE=0 to 25%) to give 22-d (137 mg, 97%) as a white solid. LC-MS (ESI): m/z=504.3 [M+H]⁺.

### Synthesis of Compound 22-c

22-d (137 mg, 0.272 mmol) was dissolved in ethyl acetate (20 mL), added MCPBA (138 mg, 0.682 mmol) and stirred at room temperature for 2 hours; LCMS monitoring indicated that the reaction was complete; the reaction was then quenched with a saturated sodium bicarbonate solution, extracted with ethyl acetate (30 mL×2), dried, concentrated and subjected to column (biotage, 25 g, silica gel, UV254, EA:PE=0 to 50%) to give 22-c (130 mg, 89%) as a white solid. LC-MS (ESI): m/z=558.2 [M+Na]⁺.

### Synthesis of Compound 22-b

22-c (130 mg, 0.243 mmol) was dissolved in toluene (15 mL): in an ice bath. N-methyl-L-prolinol (55.9 mg, 0.486 mmol) and sodium tert-butoxide (47 mg, 0.489 mmol) were added: under nitrogen atmosphere, the mixture was stirred at this temperature for 30 minutes: LCMS monitoring indicated that the reaction was complete; and then, the reaction was directly mixed with silica gel and subjected to column (biotage, 25 g, silica gel, UV254, MeOH:DCM=0 to 10%) to give 22-b (65 mg, 47%) as a white solid. LC-MS (ESI); m/z=571.3 [M+H]⁺.

### Synthesis of Compound 22-a

22-b (65 mg, 0.114 mmol) was dissolved in dichloromethane (10 mL), added trifluoroacetic acid (0.5 mL) and stirred at room temperature for 6 hours; LCMS monitoring indicated that the reaction was complete; trifluoroacetic acid was then removed, and saturated sodium bicarbonate solution was added; the mixture was extracted with dichloromethane (20 mL×2), dried, and filtered to give the filtrate, i.e., 22-a (solution), which was used directly in the next reaction. LC-MS (ESI): m/z=471.3 [M+H]⁺.

### Synthesis of Compound 22

To 22-a solution, acryloyl chloride (15.4 mg, 0.171 mmol) and DIPEA (73.6 mg, 0.57 mmol) were added; under nitrogen atmosphere, the mixture was stirred at room temperature for 2 hours; LCMS monitoring indicated that the reaction was complete; the reaction was then quenched with a saturated sodium bicarbonate solution, extracted with dichloromethane (40 mL×2), dried, concentrated and subjected to column (ISCO, 25 g, silica gel, UV254. MeOH:DCM=0 to 10%) to give 22 (20 mg, 33% yield for two steps) as a solid. LC-MS (ESI): m/z=525.3 [M+H]⁺; ¹H NMR (400 MHz, CD₃OD): δ 7.73-7.87 (m, 3H), 7.48 (t, 1H, J=7.2 Hz), 7.33-7.40 (m, 2H), 6.52-6.69 (m, 1H), 6.31-6.39 (m, 1H), 5.87-6.01 (m, 2H), 5.71-5.80 (m, 1H), 4.85-4.97 (m, 2H), 4.65-4.78 (m, 1H), 4.23-4.48 (m, 2H), 3.73-3.93 (m, 2H), 3.39-3.52 (m, 1H), 3.26-3.36 (m, 1H), 3.10-3.21 (m, 1H), 2.92 (d, 3H, J=2.0 Hz), 2.75 (s, 3H), 2.56-2.68 (m, 1H), 2.43-2.55 (m, 1H), 1.87-2.27 (m, 7H).

### Example 23 Synthetic Route of Compound 23

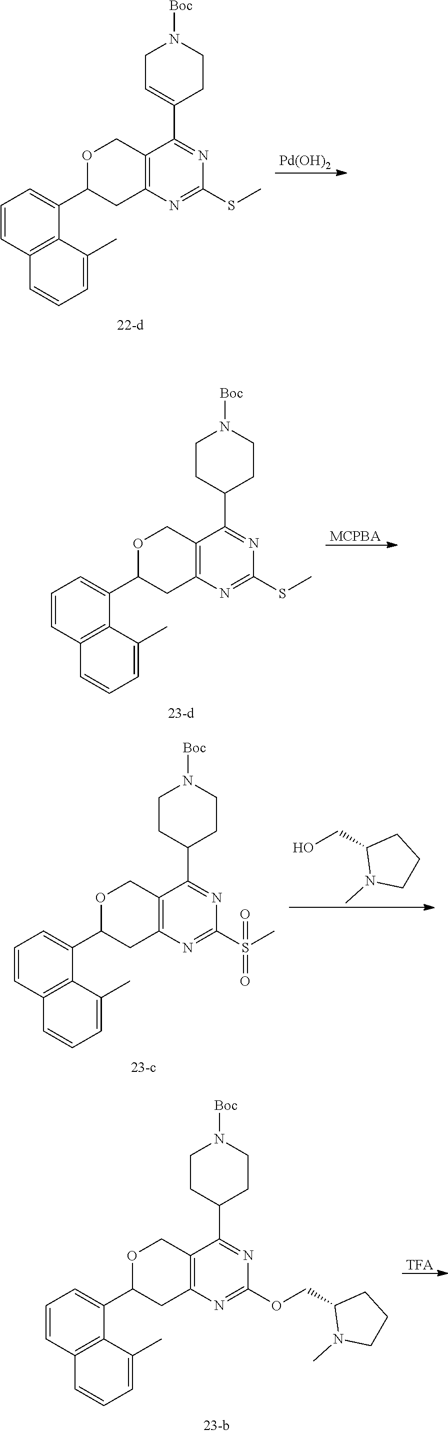

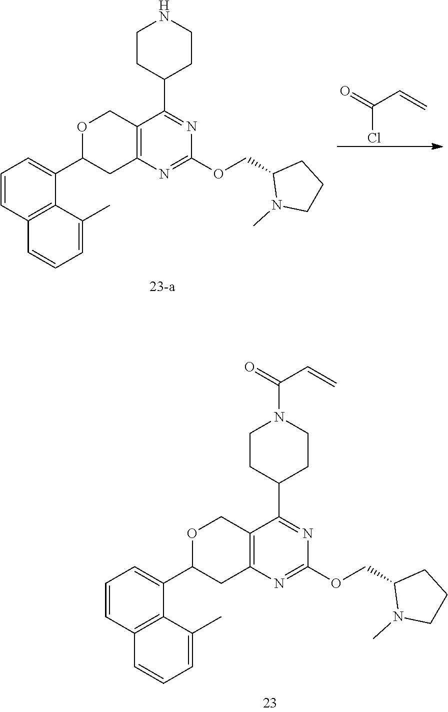

### Synthesis of Compound 23-d

22-d (200 mg, 0.398 mmol) was dissolved in methanol (100 mL), added palladium hydroxide (200 mg), replaced three times with hydrogen and stirred at 50° C. for 2 hours; LCMS monitoring the reaction was incomplete, and palladium hydroxide (200 mg) was then supplemented: the mixture was continuously reacted for 2 hours, and the reaction was incomplete; palladium hydroxide (200 mg) was then supplemented, and the reaction was continued for 2 hours; LCMS monitoring indicated that the reaction was complete; the reaction was then filtered, concentrated and subjected to column (biotage, 25 g, silica gel, UV254. EA:PE=0 to 20%) to give 23-d (80 mg, 40%) as a white solid. LC-MS (ESI); m/z=506.3 [M+H]⁻.

### Synthesis of Compound 23-c

23-d (80 mg, 0.158 mmol) was dissolved in ethyl acetate (30 mL), added MCPBA (80 mg, 0.395 mmol) and stirred at room temperature for 2 hours; LCMS monitoring indicated that the reaction was complete; the reaction was then quenched with a saturated sodium bicarbonate solution, extracted with ethyl acetate (30 mL×2), dried and concentrated to give 23-c (85 mg, 100%) as a white solid, which was used directly in the next reaction.

### Synthesis of Compound 23-b

23-c (85 mg, 0.158 mmol) was dissolved in toluene (20 mL); in an ice bath. N-methyl-L-prolinol (36.4 mg, 0.316 mmol) and sodium tert-butoxide (30 mg, 0.313 mmol) were added: under nitrogen atmosphere, the mixture was stirred at this temperature for 30 minutes: LCMS monitoring indicated that the reaction was complete; the reaction was then quenched with water, extracted with ethyl acetate (30 mL×2), dried, concentrated and subjected to column (biotage, 12 g, silica gel, UV254. MeOH:DCM=0 to 10%) to give 23-b (52 mg, 57%) as a white solid. LC-MS (ESI): m/z=573.3 [M+H]⁺.

### Synthesis of Compound 23-a

23-b (52 mg, 0.091 mmol) was dissolved in dichloromethane (10 mL), added trifluoroacetic acid (1 mL) and stirred at room temperature for 2 hours; LCMS monitoring indicated that the reaction was complete; and then trifluoroacetic acid was removed at room temperature, and saturated sodium bicarbonate solution was added: the mixture was extracted with dichloromethane (30 mL×3), dried, filtered and concentrated to give 23-a (43 mg, 100%) as a solid, which was used directly in the next reaction. LC-MS (ESI): m/z=473.3 [M+H]⁺.

### Synthesis of Compound 23

23-a (43 mg, 0.091 mmol) was dissolved in dichloromethane (20 mL) and added acryloyl chloride (12.3 mg, 0.137 mmol) and DIPEA (58.8 mg, 0.456 mmol); under nitrogen atmosphere, the mixture was stirred at room temperature for 2 hours; LCMS monitoring indicated that the reaction was complete; the reaction was then quenched with a saturated sodium bicarbonate solution, extracted with dichloromethane (30 mL×3), dried, concentrated and subjected to column (ISCO, 12 g, silica gel, UV214, MeOH:DCM=0 to 10%) to give 23 (26 mg, 54%) as a pale brown solid. LC-MS (ESI): m/z=527.3 [M+H]⁺; ¹H NMR (400 MHz, CDCl₃): δ 7.74-7.87 (m, 3H), 7.49 (t, 1H, J=7.2 Hz), 7.32-7.39 (m, 2H), 6.63 (dd, 1H, J=16.8, 10.4 Hz), 6.31 (dd, 1H, J=16.8, 1.60 Hz), 5.87-5.93 (m, 1H), 5.72 (dd, 1H, J=10.4, 1.6 Hz), 4.91-5.02 (m, 2H), 4.61-4.88 (m, 2H), 4.36-4.48 (m, 1H), 4.11-4.21 (m, 1H), 3.40-3.58 (m, 1H), 3.10-3.27 (m, 4H), 2.89 (d, 3H, J=3.2 Hz), 2.58-2.84 (m, 6H), 1.75-2.26 (m, 8H).

### Example 24 Synthetic Route of Compound 24

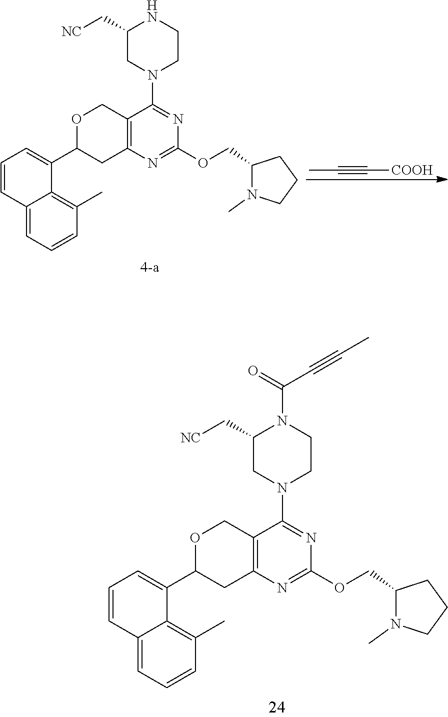

### Synthesis of Compound 24

In an ice bath, to a solution of 4-a (50 mg, 0.1 mmol) in DMF (5 mL) were successively added triethylamine (0.034 mL, 0.24 mmol), 2-tetrolic acid (12.3 mg, 0.15 mmol) and 1-propyl phosphoric anhydride (46.5 mg, 0.073 mmol). After completion of the addition, the reaction mixture was warmed to room temperature and stirred overnight. After completion of the addition, the solid was precipitated out by slowly adding water, collected by filtration, washed with water and dried to give a crude product, which was then purified by silica gel column chromatography (DCM/MeOH 10/1) to give product 24 (20 mg, 35% yield) as a white solid. LC-MS (ESI): m/z=579.0 [M+H]⁺: ¹H NMR (400 MHz, CDCl₃); δ 7.74-7.84 (m, 3H), 7.44-7.52 (m, 1H), 7.31-7.40 (m, 2H), 5.98-6.01 (m, 1H), 4.65-5.03 (m, 3H), 4.44-4.56 (m, 1H), 4.31-4.38 (m, 1H), 4.10-4.30 (m, 1H), 3.90-4.08 (m, 1H), 3.61-3.84 (m, 1H), 3.36-3.53 (m, 1H), 2.96-3.32 (m, 5H), 2.92 (d, 3H, J=12.8 Hz), 2.67-2.88 (m, 2H), 2.57 (s, 3H), 2.28-2.65 (m, 2H), 2.00-2.17 (m, 4H), 1.69-1.99 (m, 3H).

### Example 25 Synthetic Route of Compound 25

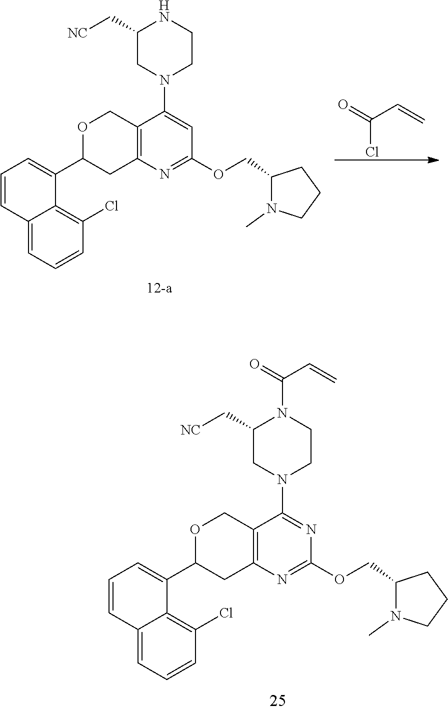

### Synthesis of Compound 25

At room temperature, compound 12-a (55 mg, 0.103 mmol) was dissolved in DCM (10 mL), and then DIPEA (85 uL, 0.515 mmol) and acryloyl chloride (14 mg, 0.155 mmol) were successively added. After completion of the addition, under nitrogen atmosphere, the reaction mixture was stirred at room temperature for 3 hours and then quenched with a saturated sodium bicarbonate solution (50 mL) and extracted with DCM (50 mL*2). The organic phase was dried over anhydrous sodium sulfate, filtered and concentrated to give a crude product: the crude product was separated and purified though a flash column chromatography (MeOH/DCM=1/10) to give compound 25 (30 mg, 50% yield) as a white solid. LC-MS (ESI): m/z=587.3 [M+1]⁺: ¹H NMR (400 MHz, CDCl₃): δ 7.96 (d, 1H, J=7.2 Hz), 7.83 (t, 2H, J=9.2 Hz), 7.64-7.49 (m, 2H), 7.36 (t, 1H, J=7.6 Hz), 6.68-6.64 (m, 2H), 6.39 (d, 1H, J=16.8 Hz), 6.31-6.04 (m, 1H), 5.83 (d, 1H, J=10.4 Hz), 5.73-4.90 (m, 2H), 4.89-4.79 (m, 1H), 4.79-4.68 (m, 1H), 4.45-4.31 (m, 1H), 4.16-3.64 (m, 3H), 3.66-3.43 (m, 3H), 3.30-3.16 (m, 2H), 3.00-2.79 (m, 3H), 2.74 (d, 3H, J=8.4 Hz), 2.63 (t, 1H, J=8.4 Hz), 2.26-2.13 (m, 1H), 2.13-2.00 (m, 1H), 2.00-1.84 (m, 2H).

### Example 26 Synthetic Route of Compound 30

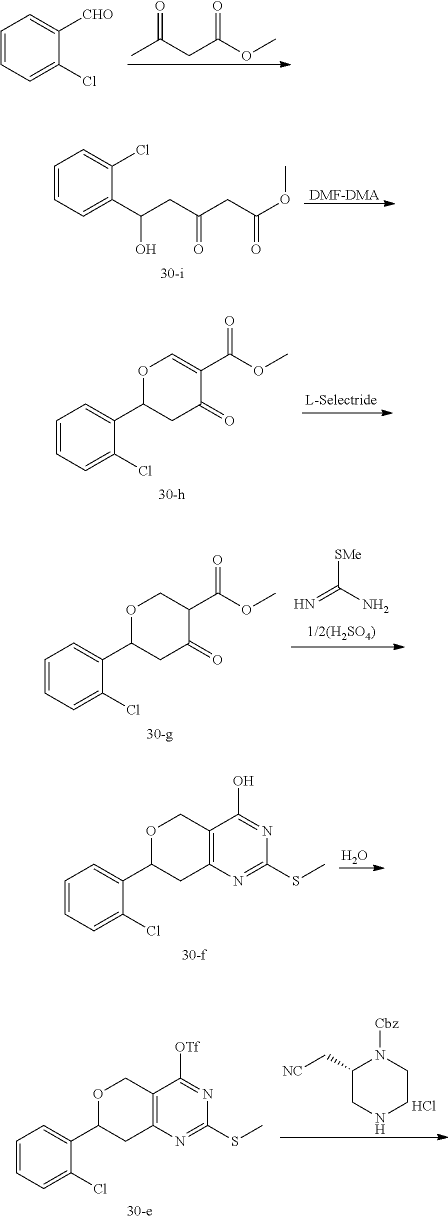

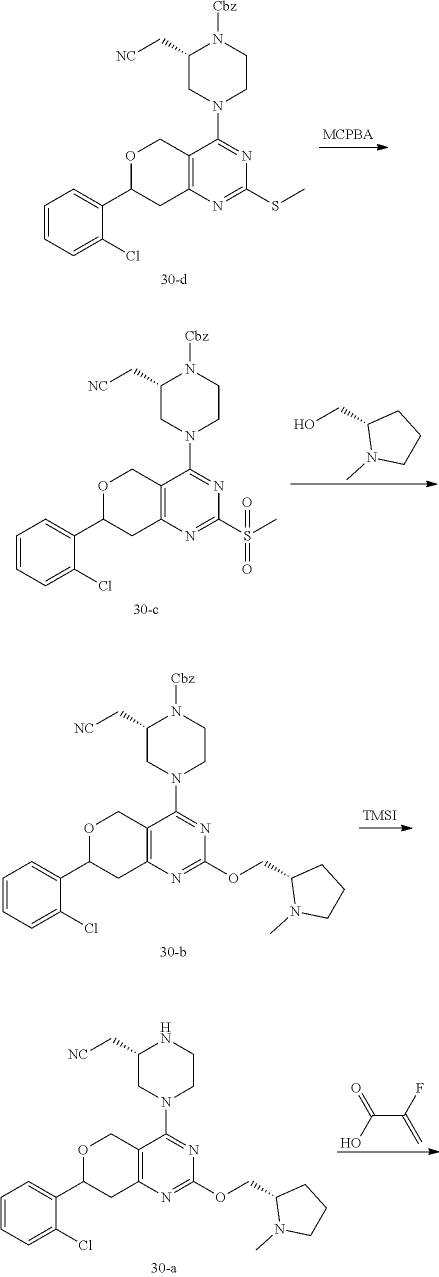

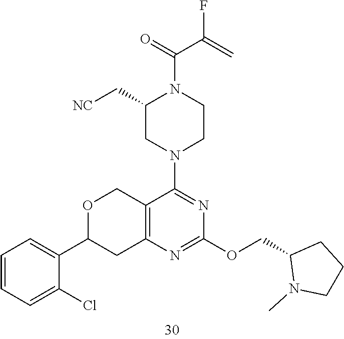 [SMILES: C=C(F)C(=O)N1CCN(c2nc(OC[C@@H]3CCCN3C)nc3c2COC(c2ccccc2Cl)C3)C[C@@H]1CC#N]

### Synthesis of Compound 30-i

At room temperature, NaH (60%, 4.26 g, 106.54 mmol) was added to 150 mL of THF, and then under nitrogen atmosphere, methyl acetoacetate (11.49 mL, 106.54 mmol) was added. Under nitrogen atmosphere, the mixture was stirred at room temperature for 30 minutes, and then n-BuLi (2.5 M, 42.62 mL, 106.54 mmol) was added dropwise at −15° C., to −10° C.; the mixture was kept at this temperature for 30 minutes, and a solution of compound o-chlorobenzaldehyde (4.0 mL, 35.51 mmol) in THF (20 mL) was then added dropwise. The mixture was stirred at low temperature (−10° C., to 0° C.) for 2 hours; the reaction was then quenched with a saturated ammonium chloride solution (100 mL) and extracted with ethyl acetate (100 mL*3); the organic phase was washed with saturated brine (100 mL), dried over anhydrous sodium sulfate, filtered and concentrated to give a crude product; the crude product was purified through a flash column chromatography to give 30-i (7.9 g, 87%) as a pale yellow oil.

### Synthesis of Compound 30-h

Compound 30-i (7.9 g, 30.78 mmol) was dissolved in DCM (250 mL): under nitrogen atmosphere, DMF-DMA (4.9 mL, 36.93 mmol) was then added at room temperature; the mixture was stirred at room temperature for 45 minutes and then added BF₃.Et₂O (4.6 mL, 36.93 mmol); and then the mixture was stirred at room temperature for 1 hour and then diluted with 200 ml, of dichloromethane; the organic phase was washed successively with a saturated NaHCO₃ solution (400 mL) and saturated brine (200 mL), dried over anhydrous sodium sulfate, filtered and concentrated to give a crude product. The crude product was purified through a flash column chromatography to give 30-h (7.5 g, 91%) as a yellow oil. LC-MS (ESI): m/z=267.0 [M+1]⁺.

### Synthesis of Compound 30-g

Compound 30-h (7.5 g, 28.12 mmol) was dissolved in THF (200 mL); at −78° C. under nitrogen atmosphere, a solution of lithium tri-sec-butyl borohydride in tetrahydrofuran (1 M, 30.94 mL, 30.94 mmol) was then added dropwise: the mixture was stirred at this temperature for 1 hour, and then the reaction was quenched with saturated ammonium chloride (100 mL) and extracted with ethyl acetate (100 mL*3); the organic phase was concentrated to give a crude product, which was purified through a flash column chromatography to give 30-g (5 g, 66%) as a yellow oil. LC-MS (ESI): m/z=269.0 [M+1]⁺.

### Synthesis of Compound 30-f

Compound 30-g (5 g, 18.6 mmol) was dissolved in methanol (30 mL), and then at room temperature, sodium carbonate (13.8 g, 111.7 mmol) and compound 2-methyl-2-thiourea sulfate (10.4 g, 37.2 mmol) were successively added. The mixture was stirred at room temperature overnight. After completion of the reaction, the pH was adjusted to 5 with 1 N dilute hydrochloric acid, and then white solid was precipitated out, filtered, washed with water, dried over anhydrous sodium sulfate, filtered and concentrated to give 30-f (4 g, 70%) as a white solid. LC-MS (ESI): m/z=309.1 [M+1]⁺.

### Synthesis of Compound 30-e

In an ice bath, to a solution of compound 30-f (4.0 g, 12.9 mmol) in DCM (100 mL) were respectively added DIPEA (4.28 ml 25.9 mmol) and trifluoromethanesulfonic anhydride (3.3 mL, 19.4 mmol). After completion of the addition, the reaction mixture was slowly warmed to room temperature and stirred for 2 hours. The reaction mixture was quenched by adding saturated aqueous sodium bicarbonate solution and extracted with ethyl acetate. The organic phase was washed with saturated aqueous sodium chloride solution, dried over anhydrous sodium sulfate, filtered and subjected to rotary evaporation to give a crude product compound, which was purified through a flash column chromatography to give 30-e (4 g, 70%) as a white solid. LC-MS (ESI): m/z=441.0 [M+1]⁺.

### Synthesis of Compound 30-d

At room temperature, to a solution of 30-e (4.0 g, 9.07 mmol) in DMF (10 mL) were respectively added N,N-diisopropylethylamine (4.5 mL, 27.2 mmol) and benzyl (S)-2-cyanomethylpiperazine-1-carboxylate hydrochloride (2.95 g, 9.98 mmol). After completion of the addition, the reaction mixture was heated to 100° C., and stirred for two hours. After completion of the reaction, the reaction mixture was diluted by adding ethyl acetate, washed successively with water and saturated brine, dried over anhydrous sodium sulfate, filtered and subjected to rotary evaporation. The crude product was separated and purified through a flash column chromatography to give 30-d (4 g, 80%) as a white solid. LC-MS (ESI): m/z=550.2 [M+H]⁺.

### Synthesis of Compound 30-c

In an ice bath, compound 30-d (0.4 g, 0.73 mmol) was dissolved in ethyl acetate (20 mL) and then added m-chloroperoxybenzoic acid (369.1 mg, 1.82 mmol); the mixture was slowly warmed to room temperature and stirred for 2 hours. After completion of the reaction, the reaction mixture was neutralized by adding saturated aqueous sodium bicarbonate solution and extracted with dichloromethane (15 mL*2); the organic phase was combined, dried over anhydrous sodium sulfate, filtered and concentrated; the crude product was purified through a flash column chromatography (DCM/MeOH=10/1) to give 30-c (0.35 g, 83%) as a white solid. LC-MS (ESI): m/z=582.2 [M+1]⁺.

### Synthesis of Compound 30-b

In an ice bath, to a solution of 30-c (350 mg, 0.6 mmol) and N-methyl-L-prolinol (138.5 mg, 1.2 mmol) in toluene (10 mL) was added sodium tert-butoxide (115.6 mg, 1.2 mmol). After completion of the addition, the reaction mixture was stirred in an ice bath for 10 minutes. After completion of the addition, the reaction mixture was concentrated under reduced pressure, diluted with ethyl acetate, washed with water and saturated brine, dried over anhydrous sodium sulfate, filtered and subjected to rotary evaporation to give a brown oil, which was purified through a flash column chromatography to give 30-b (300 mg, 80%) as a tawny oil. LC-MS (ESI): m/z=617.2 [M+H]⁺.

### Synthesis of Compound 30-a

At room temperature, to a solution of 30-b (300 mg, 0.49 mmol) in acetonitrile (40 mL) was added trimethyliodosilane (0.35 mL, 2.43 mmol). After completion of the addition, the reaction mixture was heated to 30° C., and stirred for 5 hours. After completion of the addition, the reactant was neutralized with triethylamine (10 mL) and concentrated under reduced pressure to give 30-a (200 mg, 85%) as a dark grey solid. LC-MS (ESI): m/z=483.1 [M+H]⁺.

### Synthesis of Compound 30

At room temperature, to a solution of 2-fluoroacrylic acid (55.9 mg, 0.62 mmol) and HATU (314.9 mg, 0.83 mmol) in DMF (5 mL) were added N,N-diisopropylethylamine (0.21 mL, 1.24 mmol) and 30-a (200 mg, 0.41 mmol). The reaction mixture was stirred at room temperature overnight. After completion of the reaction, the solid was precipitated out by slowly adding water, collected by filtration, washed with water and dried to give a crude product, which was then purified through a flash column chromatography (DCM/MeOH 10/1) to give product 30 (100 mg, 44%) as a white solid. LC-MS (ESI): m/z=555.0 [M+H]⁺: ¹H NMR (400 MHz, CDCl₃): δ 7.55 (d, 1H, J=7.2 Hz), 7.36-7.39 (m, 1H), 7.30-7.35 (m, 1H), 7.24-7.28 (m, 1H), 5.36 (d, 1H, J=49.6 Hz), 5.15-5.26 (m, 2H), 4.40-4.92 (m, 5H), 3.45-4.02 (m, 6H), 3.08-3.38 (m, 5H), 3.03 (d, 3H, J=13.2 Hz), 2.27-2.86 (m, 2H), 2.32-2.38 (m, 1H), 2.03-2.20 (m, 3H).

### Example 27 Synthetic Route of Compound 31

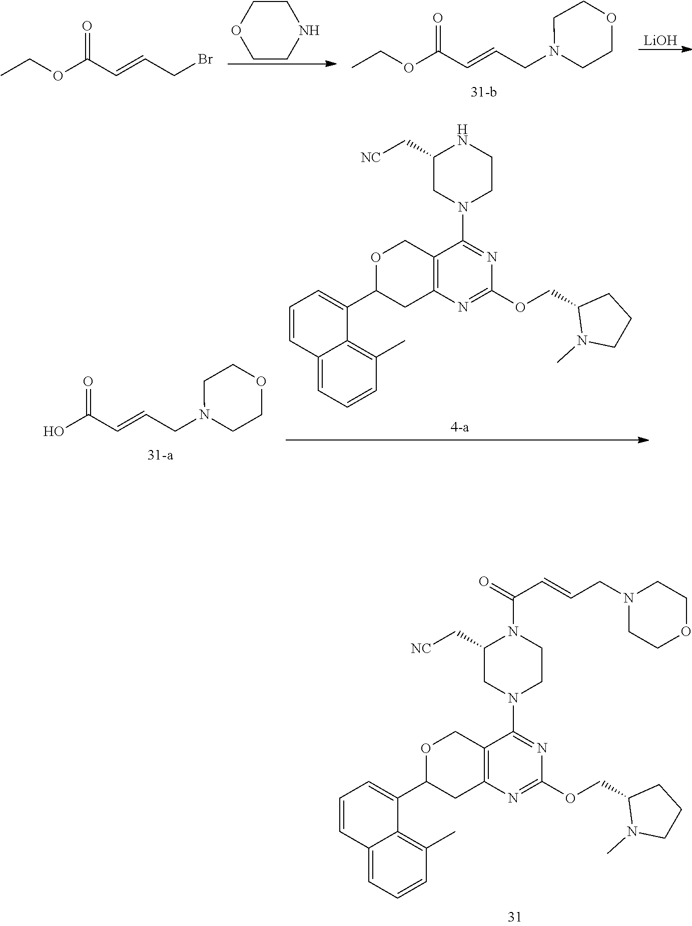

### Synthesis of Compound 31-b

In an ice bath, to a solution of compound ethyl 4-bromocrotonate (0.5 mL, 4.25 mmol) in DCM (10 mL) were respectively added potassium carbonate (1.18 g, 8.5 mmol), potassium iodide (40 mg, 0.21 mmol) and morpholine (0.56 mL, 6.38 mmol). After completion of the addition, the reaction mixture was stirred at 0° C. for 30 minutes and then slowly warmed to room temperature and stirred for 4 hours. After completion of the addition, the reaction was quenched by adding water and extracted with ethyl acetate; the organic phase was washed with saturated aqueous sodium chloride solution, dried over anhydrous sodium sulfate, filtered and subjected to rotary evaporation to give a crude product compound, which was purified through a flash column chromatography to give 31-b (0.75 g, 95%) as a tawny oil. LC-MS (ESI): m/z=200.0 [M+1]⁺.

### Synthesis of Compound 31-a

To a reaction flask, compound 31-b (750 mg, 3.76 mmol), lithium hydroxide (632 Ma, 15.1 mmol), tetrahydrofuran (12 ml), methanol (6 ml) and water (6 ml) were added. The mixture was stirred at room temperature overnight. After completion of the addition, the organic solvent was removed under reduced pressure; the aqueous layer was added dilute hydrochloric acid to adjust pH to 6-7 and extracted with ethyl acetate; the organic phase was washed successively with water and saturated aqueous sodium chloride solution, dried over anhydrous sodium sulfate, filtered and subjected to rotary evaporation to give crude product compound 31-a (500 mg, 78%) as a grey solid. LC-MS (ESI): m/z=172.0 [M+H]⁺.

### Synthesis of Compound 31

At room temperature, to a solution of 31-a (25 mg, 0.15 mmol) and HATU (74.2 mg, 0.2 mmol) in DMF (5 mL) were added N,N-diisopropylethylamine (37.8 mg, 0.3 mmol) and 4-a (50 mg, 0.1 mmol). The reaction mixture was stirred at room temperature overnight. After completion of the reaction, the solid was precipitated out by slowly adding water, collected by filtration, washed with water and dried to give a crude product. The crude product was purified through a flash column chromatography (DCM/MeOH 5/1) to give product 31 (15 mg, 23%) as a white foamy solid. LC-MS (ESI): m/z=666.0 [M+H]⁺: ¹H NMR (400 MHz, CDCl₃): δ 7.74-7.84 (m, 3H), 7.45-7.52 (m, 1H), 7.32-7.39 (m, 2H), 6.87-6.99 (m, 1H), 6.36-6.52 (m, 1H), 5.97-6.07 (m, 1H), 4.93-5.11 (m, 1H), 4.64-4.93 (m, 2H), 4.32-4.60 (m, 2H), 3.80-4.28 (m, 2H), 3.32-3.81 (m, 6H), 2.97-3.31 (m, 6H), 2.93 (d, 3H, J=8.4 Hz), 2.56-2.83 (m, 6H), 2.48 (s, 3H), 2.34-2.55 (m, 1H), 2.14-2.76 (m, 1H), 1.86-2.11 (m, 4H).

### Example 28 Synthetic Route of Compound 34

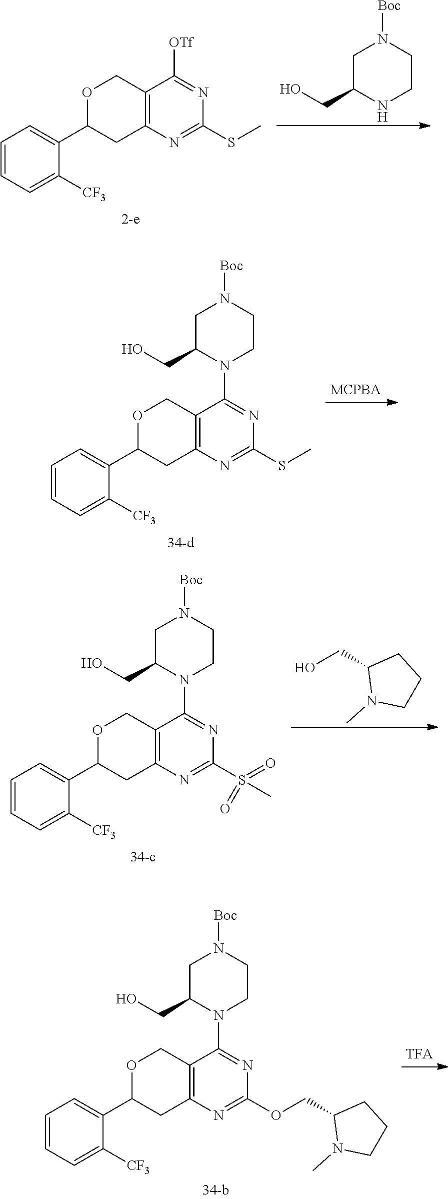

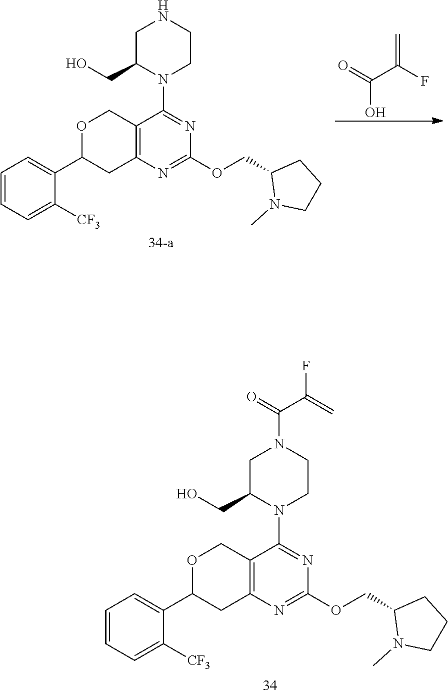

### Synthesis of Compound 34-d

At room temperature, to a solution of 2-e (205 mg, 0.43 mmol) and (R)-1-130C-3-hydroxyethylpiperazine (140 mg, 0.65 mmol) in DMF (8 mL) was added DIPEA (111 mg, 0.86 mmol). After completion of the addition, the reaction temperature was slowly increased to 100° C.; the reaction mixture was stirred at this temperature for 1 hour and then cooled to room temperature, added water and extracted with ethyl acetate (50 mL); the organic phase was washed with brine, dried over anhydrous sodium sulfate, filtered and subjected to rotary evaporation: the crude product was separated and purified through a flash column chromatography (0-50%, EA/PE) to give compound 34-d (111 mg, 47%) as a white solid. LC-MS (ESI): m/z=541.2 [M+H]⁺.

### Synthesis of Compound 34-c

In an ice bath, to a solution of 34-d (111 mg, 0.21 mmol) in ethyl acetate (8 mL) was added 85% m-chloroperoxybenzoic acid (104 mg, 0.51 mmol). The reaction was slowly warmed to room temperature, stirred for 3 hours and then added saturated aqueous sodium bicarbonate solution and extracted with ethyl acetate (30 mL*2); the combined organic phase was dried over anhydrous sodium sulfate, filtered and subjected to rotary evaporation to give crude product compound 34-c (118 mg, 100%) as a white solid. LC-MS (ESI): m/z=572.8 [M+H]⁺.

### Synthesis of Compound 34-b

At room temperature, to a solution of 34-c (118 mg, 0.21 mmol) in toluene (8 mL) were added N-methyl-L-prolinol (36 mg, 0.31 mmol) and then sodium tert-butoxide (40 mg, 0.41 mmol); the mixture was stirred at room temperature for 3 hours and then concentrated; the crude product was separated and purified by Pre-TLC (DCM:MeOH=10:1) to give compound 34-b (56 mg, 45%) as a yellow solid. LC-MS (ESI): m/z=608.3 [M+H]⁺.

### Synthesis of Compound 34-a

At room temperature, to a solution of 34-b (56 mg, 0.09 mmol) in dichloromethane (3 mL) was added TFA (1 mL); the mixture was stirred at room temperature for 1 hour and then concentrated, added saturated aqueous sodium bicarbonate solution, extracted with ethyl acetate (30 mL); the organic phase was dried over anhydrous sodium sulfate, filtered and subjected to rotary evaporation to give crude product compound 34-a (47 mg, 100%) as a yellow solid. LC-MS (ESI): m/z=508.3 [M+H]⁺.

### Synthesis of Compound 34

At room temperature, to a solution of 34-a (47 mg, 0.09 mmol) and 2-fluoroacrylic acid (13 mg, 0.14 mmol) in DMF (5 mL) were added HATU (68 mg, 0.18 mmol) and DIPEA (35 mg, 0.27 mmol); the reaction temperature was increased to room temperature: the mixture was stirred at room temperature for 1 hour and then concentrated, added water and extracted with ethyl acetate (30 mL*2); the combined organic phase was dried over anhydrous sodium sulfate, filtered and subjected to rotary evaporation: the crude product was separated and purified by Pre-HPLC to give compound 34 (12 mg, 22%) as a white solid. LC-MS (ESI); m/z=580.2 [M+H]⁺: ¹H NMR (400 MHz, CDCl₃): δ 7.75 (d, J=7.6 Hz, 1H), 7.69-7.62 (m, 2H), 7.45 (t. J=7.6 Hz, 1H), 5.40 (d, J=46 Hz, 1H), 5.28-5.14 (m, 2H), 4.86 (dd, J=14.8 Hz, 1H), 4.74 (dd, J=13.6, 5.6 Hz, 1H), 4.62-4.36 (m, 2H), 4.23-4.13 (m, 2H), 4.09-3.94 (m, 1H), 3.84 (dd, J=11.6, 7.2 Hz, 1H), 3.72-3.22 (m, 4H), 3.17-2.86 (m, 4H), 2.77-2.66 (m, 1H), 2.48 (d, J=4.8 Hz, 3H), 2.31 (dd, J=16.4, 9.2 Hz, 1H), 2.15-1.98 (m, 2H), 1.91-1.66 (m, 3H).

### Example 29 Synthesis of Comparative Compound

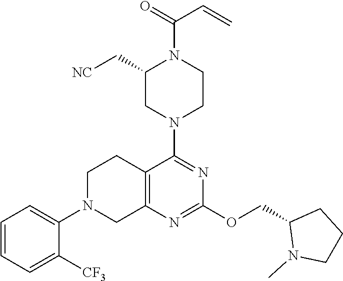 [SMILES: C=CC(=O)N1CCN(c2nc(OC[C@@H]3CCCN3C)nc3c2CCN(c2ccccc2C(F)(F)F)C3)C[C@@H]1CC#N]

Comparative compound 1′ was synthesized with reference to the method in WO 2017201161 A1.

### Example 30 Synthesis of Comparative Compound 2′

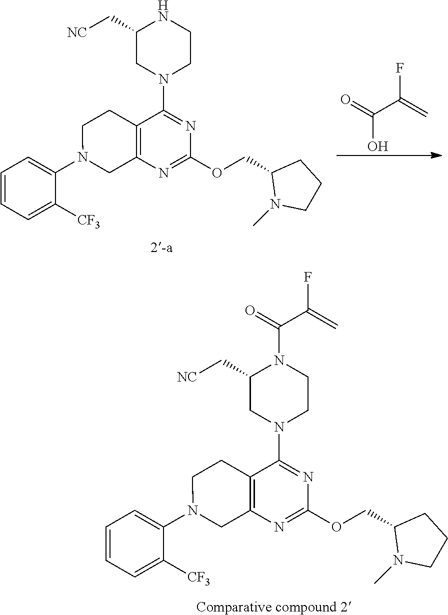

Compound 2′-a was synthesized with reference to the method in WO 2017201161 A1.

2′-a (44 mg, 0.085 mmol) was dissolved in DMF (10 mL), and 2-fluoroacrylic acid (15.4 mg, 0.171 mmol). HATU (65 mg, 0.171 mmol) and DIPEA (70.5 μL, 0.427 mmol) were successively added: under nitrogen atmosphere, the mixture was stirred at room temperature overnight. LCMS monitoring indicated that the reaction was complete; the reaction was then quenched with a saturated sodium bicarbonate solution and extracted with ethyl acetate (30 mL×3); the organic phase was washed with saturated brine (30 mL×3), dried, concentrated and subjected to column (biotage, 25 g, silica gel, UV254. MeOH:DCM=0 to 10%) to give comparative compound 2′ (49 mg, 98%) as a pale yellow solid. LC-MS (ESI); m/z=587.9 [M+H]⁺: ¹H NMR (400 MHz, CDCL₃): δ 7.67 (d, 1H, J=8.4 Hz), 7.59 (t, 1H, J=7.6 Hz), 7.44 (d, 1H, J=8.0 Hz), 7.30 (t, 1H, J=8.0 Hz), 5.38 (d, 1H, J=47.2 Hz), 5.24 (dd, 1H, J=4.0, 16.8 Hz), 4.54-4.63 (m, 2H), 4.24-4.34 (m, 1H), 4.00-4.10 (m, 3H), 3.71-3.79 (m, 1H), 3.60-3.68 (m, 1H), 3.33-3.43 (m, 1H), 3.05-3.24 (m, 4H), 3.01 (s, 3H), 2.93-2.98 (m, 1H), 2.72-2.86 (m, 4H), 2.26-2.41 (m, 2H), 2.03-2.21 (m, 4H).

### Example 31 Synthetic Route of Compound 35

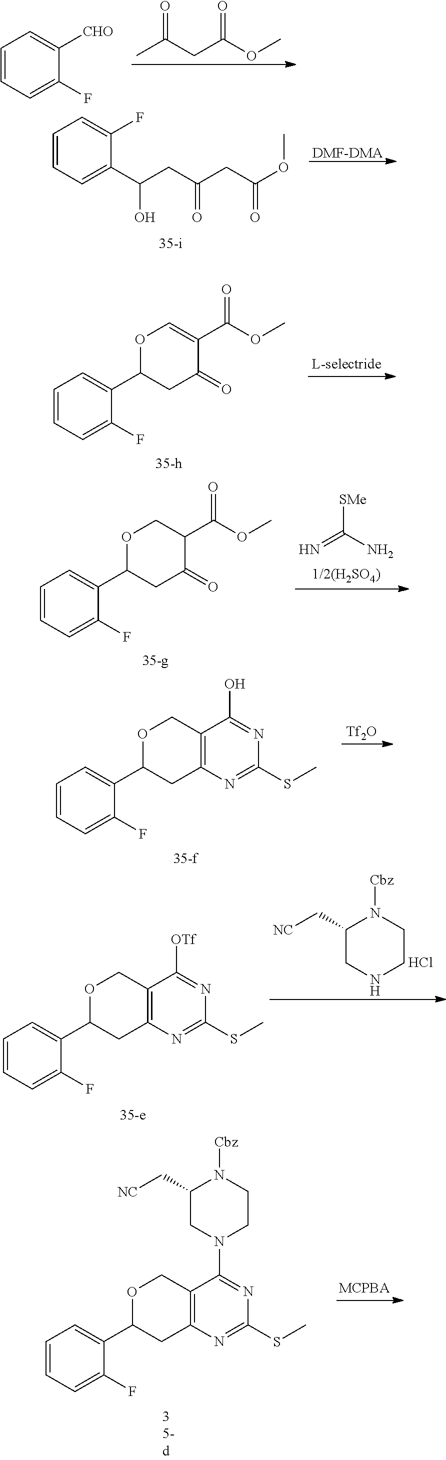

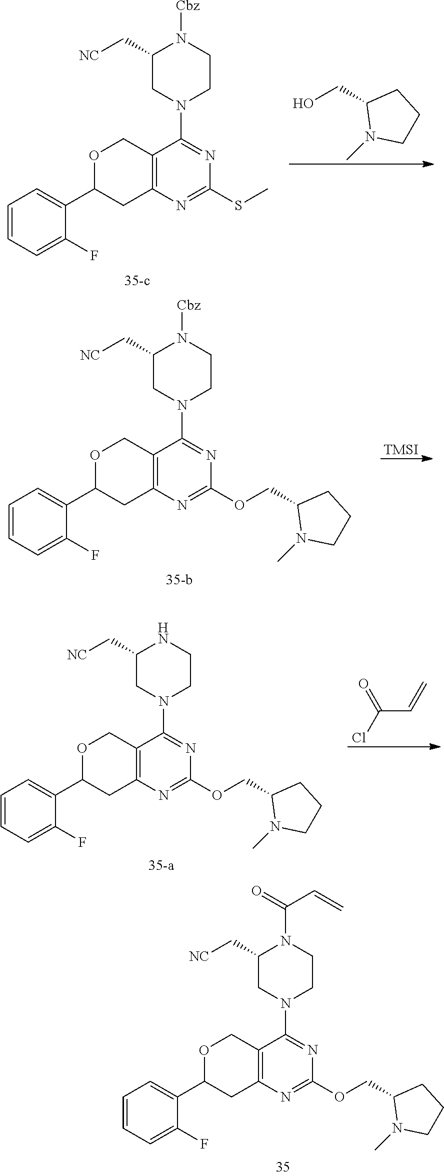

### Synthesis of Compound 35-i

At room temperature. NaH (60%, 0.97 g, 24.17 mmol) was suspended in 30 mL of THE and then under nitrogen atmosphere, methyl acetoacetate (2.61 mL, 24.17 mmol) was added. Under nitrogen atmosphere, the mixture was stirred at room temperature for 30 minutes, and then n-BuLi (2.5 M, 9.67 mL, 24.17 mmol) was added dropwise at −15° C., to −10° C.; the mixture was kept at this temperature for 30 minutes, and then a solution of compound o-fluorobenzaldehyde (1.0 g, 8.06 mmol) in THF (10 mL) was added dropwise. The mixture was stirred at low temperature (−10° C., to 0° C.) for 2 hours: the reaction was then quenched with a saturated ammonium chloride solution (20 mL) and extracted with ethyl acetate (20 mL*3); the organic phase was washed with saturated brine (20 mL), dried over anhydrous sodium sulfate, filtered and concentrated to give a crude product: the crude product was purified through a flash column chromatography to give 35-i (1.2 g, 62%) as a pale yellow oil. LC-MS (ESI): m/z=263.1 [M+Na]⁺.

### Synthesis of Compound 35-h

Compound 35-i (1.2 g, 5.0 mmol) was dissolved in DCM (50 mL); under nitrogen atmosphere, DMF-DMA (0.8 mL, 6.0 mmol) was then added at room temperature: the mixture was stirred at room temperature for 45 minutes and then added BF₃.Et₂O (0.74 mL, 6.0 mmol); and then the mixture was stirred at room temperature for 1 hour and then diluted with 20 mL of dichloromethane; the organic phase was washed successively with a saturated NaHCO₃ solution (40 mL) and saturated brine (20 mL), dried over anhydrous sodium sulfate, filtered and concentrated to give a crude product. The crude product was purified through a flash column chromatography to give 35-h (1 g, 80%) as a tawny oil. LC-MS (ESI): m/z=251.1 [M+H]⁺.

### Synthesis of Compound 35-g

Compound 35-h (1.0 g, 4.0 mmol) was dissolved in THF (30 mL): at −78° C. under nitrogen atmosphere, lithium tri-sec-butyl borohydride (1 M, 4.4 mL, 4.4 mmol) was then added dropwise: the mixture was stirred at this temperature for 1 hour, and then the reaction was quenched with saturated ammonium chloride (20 mL) and extracted with ethyl acetate (20 mL*3); the organic phase was concentrated to give a crude product, which was purified through a flash column chromatography to give 35-g (0.6 g, 60%) as a yellow oil. LC-MS (ESI): m/z=253.2 [M+1]⁺.

### Synthesis of Compound 35-f

Compound 35-g (0.6 g, 2.4 mmol) was dissolved in methanol (15 mL), and then at room temperature, sodium carbonate (1.77 g, 14.3 mmol) and compound 2-methyl-2-thiourea sulfate (1.3 g, 4.8 mmol) were successively added. The mixture was stirred at room temperature overnight. After completion of the reaction, the pH was adjusted to 5 with 1 M dilute hydrochloric acid, and then white solid was precipitated out, filtered, washed with water, dried over anhydrous sodium sulfate, filtered and concentrated to give 35-f (0.3 g, 43%) as a white solid. LC-MS (ESI): m/z=293.1 [M+1]⁺.

### Synthesis of Compound 35-e

In an ice bath, to a solution of compound 35-f (0.3 g, 1.0 mmol) in DCM (20 mL) were respectively added DIPEA (265.3 mg, 2.0 mmol) and trifluoromethanesulfonic anhydride (434.3 mg, 1.54 mmol). After completion of the addition, the reaction mixture was slowly warmed to room temperature and stirred for 2 hours. The reaction mixture was quenched by adding saturated aqueous sodium bicarbonate solution and extracted with ethyl acetate. The organic phase was washed with saturated aqueous sodium chloride solution, dried over anhydrous sodium sulfate, filtered and subjected to rotary evaporation to give a crude product compound, which was purified through a flash column chromatography to give 35-e (260 mg, 60%) as a white solid. LC-MS (ESI): m/z=425.1 [M+1]⁺.

### Synthesis of compound 35-d

At room temperature, to a solution of 35-e (0.26 g, 0.61 mmol) un DMF (10 mL) were respectively added DIPEA (0.3 mL, 1.84 mmol) and benzyl (S)-2-cyanomethylpiperazine-1-carboxylate hydrochloride (199.3 mg, 0.67 mmol). After completion of the addition, the reaction mixture was heated to 100° C., and stirred for two hours. After completion of the reaction, the reaction mixture was diluted by adding ethyl acetate, washed successively with water and saturated brine, dried over anhydrous sodium sulfate, filtered and subjected to rotary evaporation. The crude product was separated and purified through a flash column chromatography to give 35-d (0.28 g, 86%) as a white solid. LC-MS (ESI): m/z=534.2 [M+H]⁺.

### Synthesis of Compound 35-c

In an ice bath, compound 35-d (0.28 g, 0.52 mmol) was dissolved in ethyl acetate (20 mL), and then MCPBA (266.2 mg, 1.31 mmol) was added; the mixture was slowly warmed to room temperature and stirred for 2 hours. After completion of the reaction, the reaction mixture was neutralized by adding saturated aqueous sodium bicarbonate solution and extracted with dichloromethane (15 mL*2); the organic phase was combined, dried over anhydrous sodium sulfate, filtered and concentrated: the crude product was purified through a flash column chromatography (DCM/MeOH=10/1) to give 35-c (0.26 g, 88%) as a white solid. LC-MS (ESI): m/z=566.3 [M+1]⁺.

### Synthesis of Compound 35-b

In an ice bath, to a solution of 35-c (260 mg, 0.46 mmol) and N-methyl-L-prolinol (105.6 mg, 0.92 mmol) in toluene (10 mL) was added sodium tert-butoxide (88.4 mg, 0.92 mmol). After completion of the addition, the reaction mixture was stirred in an ice bath for 10 minutes. After completion of the addition, the reaction mixture was concentrated under reduced pressure, diluted with ethyl acetate, washed with water and saturated brine, dried over anhydrous sodium sulfate, filtered and subjected to rotary evaporation to give a brown oil, which was purified through a flash column chromatography to give 35-b (80 mg, 29%) as a tawny oil. LC-MS (ESI): m/z=601.0 [M+H]⁺.

### Synthesis of Compound 35-a

At room temperature, to a solution of 35-b (80 mg, 0.13 mmol) un acetonitrile (20 mL) was added trimethyliodosilane (0.1 mL, 0.67 mmol). After completion of the addition, the reaction mixture was heated to 30° C., and stirred for 5 hours. After completion of the addition, the reactant was neutralized with triethylamine (10 mL) and concentrated under reduced pressure to give 35-a (20 mg, 32%) as a dark grey solid. LC-MS (ESI): m/z=467.0 [M+H]⁺.

### Synthesis of Compound 35

At room temperature, to a solution of compound 35-a (20 mg, 0.043 mmol) in dichloromethane (10 ml) were added acryloyl chloride (7.8 mg, 0.086 mmol) and DIPEA (16.6 mg, 0.13 mmol). The reaction mixture was stirred at room temperature overnight. After completion of the addition, a crude product was obtained by concentration. The crude product was purified by preparative flash column chromatography (DCM/MeOH 10/1) to give compound 35 (5 mg, 22%) as a tawny oil. LC-MS (ESI): m/z=521.2 [M+H]⁺; ¹H NMR (400 MHz, CDCl₃): δ 7.48-7.52 (m, 1H), 7.29-7.33 (m, 1H), 7.20 (t, 1H, J=7.6 Hz), 7.08 (t, 1H, J=8.0 Hz), 6.49-6.78 (m, 1H), 6.39 (d, 1H, J=16.8 Hz), 5.82 (d, 1H, J=10.4 Hz), 4.72-5.31 (m, 4H), 4.49-4.61 (m, 1H), 3.45-4.08 (m, 5H), 2.66-3.44 (m, 10H), 2.26-2.41 (m, 1H), 2.05-2.21 (m, 1H), 1.38-1.59 (m, 4H).

### Example 32 Synthetic Route of Compound 36

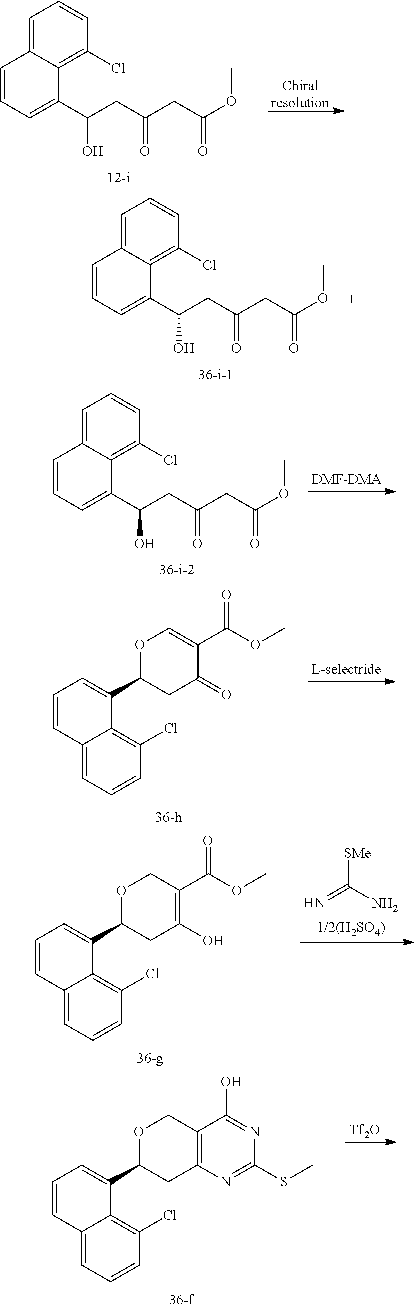

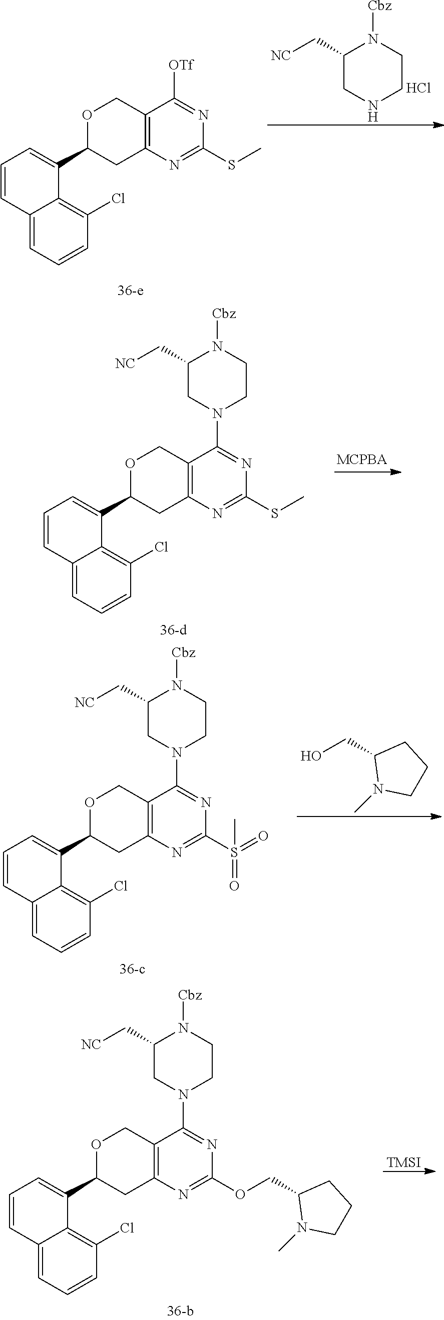

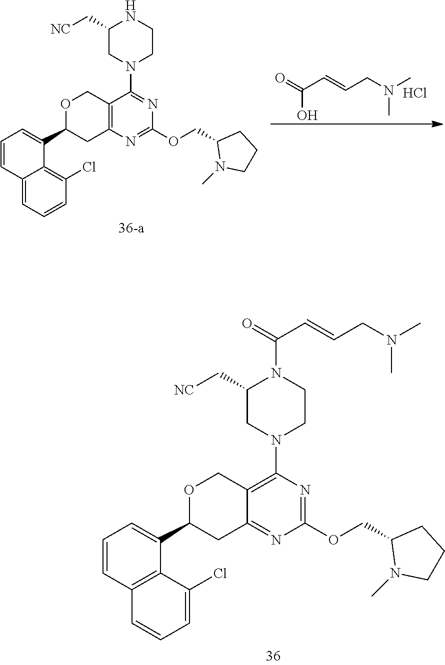

### Synthesis of Compounds 36-i-1 and 36-i-2

Compound 12-i (8.5 g, 27.8 mmol) was prepared (scale-up) and subjected to chiral resolution to give compound 364-1 (2.5 g, 29%) as a white solid and compound 36-i-2 (2.6 g, 31%) as a white solid.

| Chiral analysis conditions | Chiral preparation conditions |
|---|---|
| equipment: SFC Method Station | instrument: SFC-150 (Thar, |
| (Thar, Waters) | Waters) |
| chromatographic column: AD-H | chromatographic column: AD |
| 4.6 * 100 mm, 5 μm (Daicel) | 20 * 250 mm, 10 um (Daicel) |
| column temperature: 40° C. | column temperature: 35° C. |
| mobile phase: CO2/Ethanol (1% | mobile phase: CO2/Ethanol |
| methanol ammonia) = 75/25 | (0.2% methanol ammonia) = |
| flow rate: 4.0 ml/min | 65/35 |
| wavelength: 254 nm | flow rate: 100 g/min |
| back pressure: 120 bar | back pressure: 100 bar |
| | detection wavelength: 214 nm |
| | cycling time: 5.0 min |
| | sample solution: 8.5 g |
| | dissolved in 150 ml of methanol |
| | and dichloromethane |
| 36-i-1: retention time: 1.57 min; | |
| e.e. % = 100.0%; | |
| 36-i-2: retention time: 2.33 min; | |
| e.e. % = 99.12%. | |

36-i-1: LC-MS (ESI): m/z=329.1 [M+Na]⁺.

36-i-2: LC-MS (ESI): m/z=329.1 [M+Na]⁺.

### Synthesis of Compound 36-h

At room temperature, compound 364-1 (2.3 g, 7.5 mmol) was dissolved in DCM (80 mL), and then under nitrogen atmosphere, DMF-DMA (1.2 mL, 9.0 mmol) was added at room temperature. At room temperature, the reaction mixture was stirred for 45 minutes and then was added BF₃.Et₂O (1.2 mL, 9.0 mmol). After completion of the addition, the mixture was stirred at room temperature for 1 hour and then quenched with a saturated sodium bicarbonate solution and extracted with dichloromethane (100 mL*2); the organic phase was washed with saturated brine (100 mL,*2), dried over anhydrous sodium sulfate, filtered and concentrated to give crude product compound 36-h (2.0 g, 84%), which was used directly in the next reaction. LC-MS (ESI): m/z=317.1 [M+1]⁺.

### Synthesis of Compound 36-g

At room temperature, compound 36-h (2.0 g, 6.31 mmol) was dissolved in THF (60 mL), and then at −78° C. under nitrogen atmosphere, tri-sec-butyl lithium borohydride (1 M in THF, 6.95 mL, 6.95 mmol) was added dropwise. After completion of the addition, the mixture was stirred at −78° C. for 1 hour, and then the reaction was quenched with a 1 M hydrochloric acid solution (20 mL) and extracted with ethyl acetate (100 mL*2); the organic phase was washed with saturated brine (50 mL*2), dried over anhydrous sodium sulfate, filtered and concentrated to give a crude product: the crude product was separated and purified through a flash column chromatography (PE/EA=0 to 15%) to give compound 36-g (1.8 g, 89%) as a yellow oil. LC-MS (ESI): m/z=319.0 [M+1]⁺.

### Synthesis of Compound 36-f

At room temperature, compound 36-g (1.5 g, 4.71 mmol) was dissolved in methanol (30 mL), and then at 0° C., under nitrogen atmosphere, sodium methylate (1.27 g, 23.5 mmol) and compound 2-methyl-2-thiourea sulfate (1.18 g, 4.24 mmol) were successively added. After completion of the addition, the mixture was warmed to room temperature and stirred for 20 hours. The pH of the reaction mixture was adjusted to 5 with 1 M dilute hydrochloric acid; the solid was precipitated out and filtered; the filter cake was washed with a mixed solution of ethyl acetate (20 mL) and petroleum ether (20 mL); the solid was collected and dried in vacuum to give crude product 36-f (0.65 g, 39%) as a white solid. LC-MS (ESI); m/z=359.1 [M+1]⁺.

### Synthesis of Compound 36-e

At room temperature, compound 36-f (0.6 g, 1.67 mmol) was dissolved in DCM (50 mL), and then in an ice-water bath, under nitrogen atmosphere, DIPEA (0.83 mL, 5.02 mmol) and trifluoromethanesulfonic anhydride (0.51 mL, 3.01 mmol) were successively added. After completion of the addition, the reaction mixture was stirred in an ice-water bath for 2 hours and then quenched with a saturated sodium bicarbonate solution (50 mL) and extracted with DCM (50 mL*3); the organic phase was dried over anhydrous sodium sulfate, filtered and concentrated to give a crude product; the crude product was separated and purified through a flash column chromatography (EA/PE=0 to 5%) to give compound 36-e (380 mg, 463%) as a white solid. LC-MS (ESI): m/z=491.1 [M+1]⁺; chiral-HPLC 100% (ee %).

### Synthesis of Compound 36-d

At room temperature, compound 36-e (360 mg, 0.73 mmol) was dissolved in DMF (15 mL), and then DIPEA (0.36 mL, 2.2 mmol) and benzyl (S)-2-cyanomethylpiperazine-1-carboxylate hydrochloride (238.6 mg, 0.81 mmol) were successively added. After completion of the addition, under nitrogen protection, the mixture was stirred at 100° C. for 1 hour and then cooled to room temperature: the reaction was quenched with saturated brine (50 mL) and extracted with ethyl acetate (50 mL*2). The organic phase was washed with saturated brine (50 mL*3) and then was dried over anhydrous sodium sulfate, filtered and concentrated to give a crude product; the crude product was separated and purified through a flash column chromatography (EA/PE=0 to 40%) to give compound 36-d (360 mg, 80%) as a white solid. LC-MS (ESI): m/z=600.2 [M+1]⁺.

### Synthesis of Compound 36-c

At room temperature, compound 36-d (340 mg, 0.57 mmol) was dissolved in ethyl acetate (25 mL), and then at room temperature. MCPBA (85%, 287.5 mg, 1.42 mmol) was added. After completion of the addition, the mixture was stirred at room temperature for 2 hours, and then the reaction was quenched with a saturated sodium bicarbonate solution (20 mL) and extracted with ethyl acetate (50 mL*2); the organic phase was dried over anhydrous sodium sulfate, filtered and concentrated to give a crude product: the crude product was separated and purified through a flash column chromatography (EA/PE=0 to 60%) to give compound 36-c (320 mg, 89%) as a white solid. LC-MS (ESI): m/z=632.2 [M+1]⁺.

### Synthesis of Compound 36-b

At room temperature, compound 36-c (300 mg, 0.47 mmol) was dissolved in toluene (10 mL), and then the reaction mixture was cooled to 0° C.; N-methyl-L-prolinol (109.3 mg, 0.95 mmol) and t-BuONa (91.2 mg, 0.95 mmol) were successively added. After completion of the addition, under nitrogen atmosphere, the reaction mixture was stored in an ice-water bath for 0.5 hours and then quenched with water (20 mL) and extracted with ethyl acetate (30 mL*2). The organic phase was dried over anhydrous sodium sulfate, filtered and concentrated to give a crude product: the crude product was separated and purified through a flash column chromatography (MeOH/DCM=0 to 7%) to give compound 36-b (290 mg, 91%) as a white solid. LC-MS (ESI): m/z=667.3 [M+1]⁺.

### Synthesis of Compound 36-a

At room temperature, compound 36-b (200 mg, 0.3 mmol) was dissolved in acetonitrile (20 mL) and added trimethyliodosilane (0.21 mL, 1.5 mmol) with stirring; the mixture was stirred at 30° C. for 2 hours, quenched with triethylamine (1 mL) and concentrated to give compound 36-a (crude product) as a brown solid. LC-MS (ESI): m/z=533.3 [M+1]⁺.

### Synthesis of Compound 36

In an ice bath, to a solution of compound 36-a (50 mg, 0.094 mmol) and HATU (71.3 mg, 0.19 mmol) in DMF (5 mL) were respectively added DIPEA (60.6 mg, 0.47 mmol) and 4-(dimethylamino)-2-butenoic acid hydrochloride (23.3 mg, 0.14 mmol). The reaction mixture was stirred at room temperature overnight. After completion of the reaction, the solid was precipitated out by slowly adding water, collected by filtration, washed with water and dried to give a crude product, which was then purified by silica gel column chromatography (DCM/MeOH 5/1) to give 36 (16 mg, 26%) as a white solid. LC-MS (ESI); m/z=644.3 [M+H]⁻: ¹H NMR (400 MHz, CDCl₃): δ 7.97 (d, 1H, J=6.8 Hz), 7.83 (t, 2H, J=8.4 Hz), 7.60 (d, 1H, J=7.2 Hz), 7.56 (t, 1H, J=7.6 Hz), 7.36 (t, 1H, J=8.0 Hz), 6.91-6.97 (m, 1H), 6.51 (dd, 1H, J=11.2, 3.2 Hz), 6.37-6.55 (m, 1H), 5.00 (d, 1H, J=13.6 Hz), 4.83 (d, 1H, J=14.0 Hz), 4.55-5.10 (m, 1H), 4.47 (dd, 1H, J=10.8, 5.2 Hz), 4.21 (dd, 1H, J=10.8, 6.4 Hz), 3.99 (d, 1H, J=13.6 Hz), 3.78-3.96 (m, 1H), 3.62-3.78 (m, 1H), 3.59 (dd, 1H, J=18.4, 2.8 Hz), 3.39-3.54 (m, 1H), 3.02-3.24 (m, 4H), 2.62-2.95 (m, 5H), 2.53 (s, 3H), 2.32-2.39 (m, 1H), 2.29 (s, 6H), 2.04-2.10 (m, 1H), 1.75-1.90 (m, 3H).

### Example 33 Synthetic Route of Compound 37

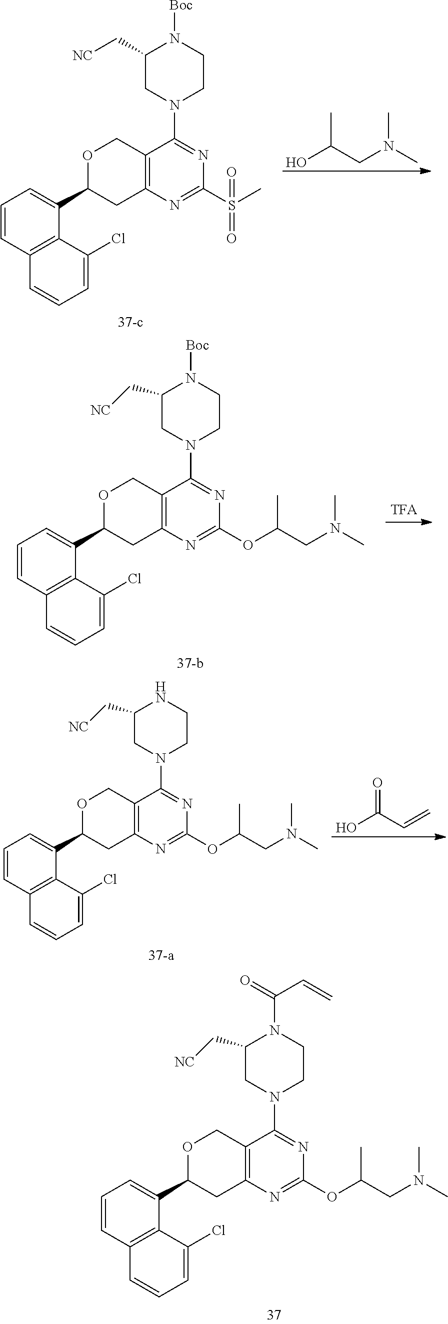

### Synthesis of Compound 37-e

37-c was synthesized with reference to the synthesis of compound 36-c, wherein benzyl (S)-2-cyanomethylpiperazine-1-carboxylate hydrochloride was replaced with tert-butyl (S)-2-cyanomethylpiperazine-1-carboxylate.

### Synthesis of Compound 37-b

In an ice bath, to a solution of 37-c (200 mg, 0.34 mmol) and 1-dimethylamino-2-propanol (68.9 mg, 0.67 mmol) in toluene (10 mL) was added sodium tert-butoxide (64.3 mg, 0.67 mmol). After completion of the addition, the reaction mixture was stirred in an ice bath for 10 minutes. After completion of the addition, the reaction mixture was concentrated under reduced pressure, diluted with ethyl acetate, washed with water and saturated brine, dried over anhydrous sodium sulfate, filtered and subjected to rotary evaporation to give a brown oil, which was purified through a flash column chromatography to give 37-b (150 mg, 72%) as a white solid. LC-MS (ESI): m/z=621.3 [M+H]⁺.

### Synthesis of Compound 37-a

To a solution of 37-b (150 mg, 0.24 mmol) in dichloromethane (10 mL) was added trifluoroacetic acid (2 mL). The resulting reaction mixture was stirred at room temperature for 4 hours. After completion of the addition, the reaction mixture was concentrated, carefully neutralized to a pH of greater than 7 with saturated sodium bicarbonate solution in an ice bath and extracted with ethyl acetate; the organic layer was combined, washed with saturated brine, dried over anhydrous sodium sulfate, filtered and concentrated to give 37-a (100 mg, 79%) as an amber-colored oil. LC-MS (ESI): m/z=521.3 [M+1]⁺.

### Synthesis of Compound 37

At room temperature, to a solution of 37-a (100 mg, 0.19 mmol) and HATU (145.9 mg, 0.38 mmol) in DMF (5 mL) were respectively added DIPEA (74.4 mg, 0.58 mmol) and acyclic acid (20.7 mg, 0.29 mmol). The reaction mixture was stirred at room temperature overnight. After completion of the reaction, the solid was precipitated out by slowly adding water, collected by filtration, washed with water and dried to give a crude product, which was then purified by silica gel column chromatography (DCM/MeOH 10/1) to give 37 (25 mg, 23%) as a white solid. LC-MS (ESI): m/z=575.2 [M+H]⁺: ¹H NMR (400 MHz, CDCl₃): δ 7.97 (d, 1H, J=7.6 Hz), 7.81-7.86 (m, 2H), 7.54-7.62 (m, 2H), 7.37 (t, 1H, J=8.0 Hz), 6.48-6.75 (m, 2H), 6.38 (d, 1H, J=16.8 Hz), 5.83 (d, 1H, J=10.8 Hz), 5.39-5.53 (m, 1H), 4.48-5.12 (m, 3H), 3.46-4.22 (m, 5H), 2.70-3.35 (m, 7H), 2.55-2.67 (m, 6H), 1.39 (dd, 3H, J=6.0, 1.6 Hz).

### Example 34 Synthetic Route of Compound 38

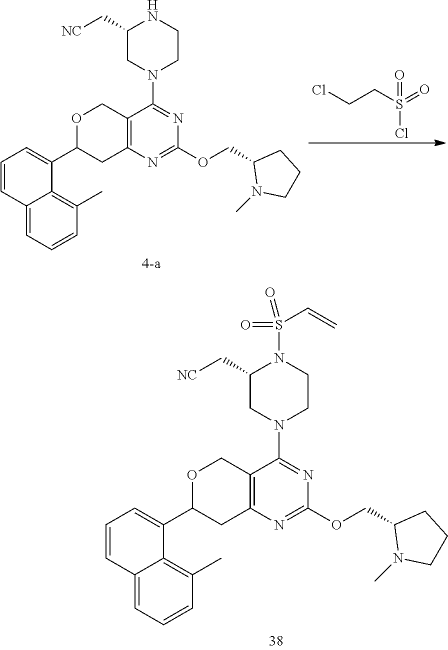 [SMILES: Cc1cccc2cccc(C3Cc4nc(OC[C@@H]5CCCN5C)nc(N5CCN[C@@H](CC#N)C5)c4CO3)c12 ; O=S(=O)(Cl)CCCl ; C=CS(=O)(=O)N1CCN(c2nc(OC[C@@H]3CCCN3C)nc3c2COC(c2cccc4cccc(C)c24)C3)C[C@@H]1CC#N]

### Synthesis of Compound 38

At room temperature, to a solution of compound 4-a (100 mg, 0.20 mmol) in dichloromethane (10 mL) were respectively added 2-chloroethanesulfonyl chloride (47.7 mg, 0.29 mmol) and triethylamine (59.3 mg, 0.59 mmol). The reaction mixture was stirred at room temperature overnight. After completion of the addition, a crude product was obtained by concentration and then purified by preparative flash column chromatography to give compound 38 (40 mg, 34%) as a white solid. LC-MS (ESI): m/z=603.3 [M+H]⁺: ¹H NMR (400 MHz, CDCl₃): δ 7.74-7.84 (m, 3H), 7.45-7.49 (m, 1H), 7.34-7.37 (m, 2H), 6.55 (dd, 1H, J=16.8, 2.8 Hz), 6.34 (dd, 1H, J=16.4, 2.0 Hz), 6.05 (dd, 1H, J=9.6, 3.6 Hz), 5.97-6.00 (m, 1H), 4.88 (dd, 1H, J=54.8, 13.6 Hz), 4.68 (dd, 1H, J=35.2, 13.2 Hz), 4.44-4.51 (m, 1H), 4.34-4.38 (m, 1H), 4.17-4.25 (m, 1H), 3.42-3.98 (m, 4H), 2.96-3.31 (m, 5H), 2.92 (d, 3H, J=12.8 Hz), 2.76-2.88 (m, 2H), 2.55 (s, 3H), 2.35-2.41 (m, 1H), 2.02-2.13 (m, 1H), 1.80-1.92 (m, 4H).

### Example 35 Synthetic Route of Compound 39

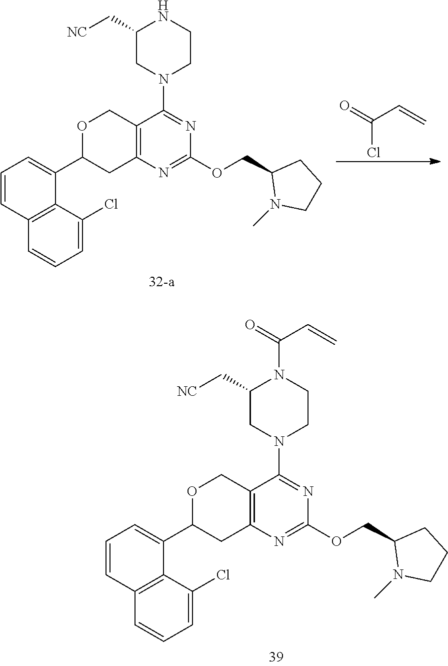

### Synthesis of Compound 39

At room temperature, compound 32-a (55 mg, 0.103 mmol) was dissolved in DCM (10 mL), and then DIPEA (85 μL, 0.515 mmol) and acryloyl chloride (14 mg, 0.155 mmol) were successively added. After completion of the addition, under nitrogen atmosphere, the reaction mixture was stirred at room temperature for 3 hours and then quenched with a saturated sodium bicarbonate solution (50 mL) and extracted with DCM (50 mL*2). The organic phase was dried over anhydrous sodium sulfate, filtered and concentrated to give a crude product: the crude product was separated and purified through a flash column chromatography (MeOH/DCM=1/10) to give compound 39 (20 mg, 33% yield). LC-MS (ESI): m/z=587.3 [M+1]⁺.

### Example 36 Synthetic Route of Compound 40

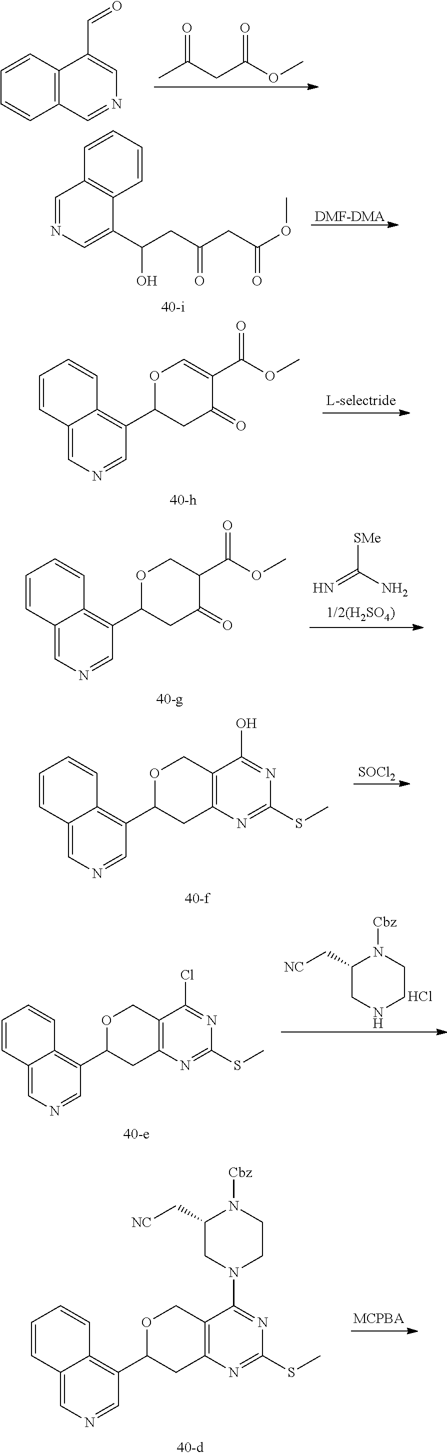

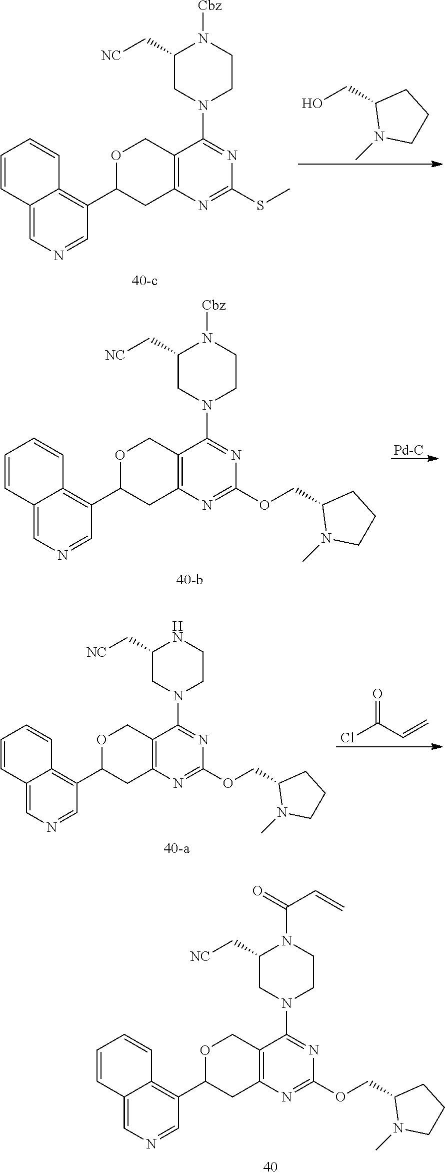

### Synthesis of compound 40-i

At room temperature, NaH (60%, 3.6 g, 90.0 mmol) was added to TUT (150 mL); under nitrogen atmosphere, methyl acetoacetate (8 mL, 77.0 mmol) was added at room temperature. Under nitrogen atmosphere, the mixture was stirred at room temperature for 30 minutes, and then n-BuLi (2.5 M, 36.0 mL, 90.0 mmol) was added dropwise at −15° C., to −10° C. After completion of the addition, the reaction mixture was kept at this temperature and stirred for 30 minutes, and then a solution of compound 4-isoquinolinecarboxaldehyde (5.0 g, 30.0 mmol) in THF (150 mL) was added dropwise. After completion of the addition, the mixture was stirred at low temperature (−10° C., to 0° C.) for 2 hours. The reaction was quenched by adding a saturated ammonium chloride solution (100 mL) and extracted with ethyl acetate (100 mL*3); the organic phase was washed with saturated brine (150 mL), dried over anhydrous sodium sulfate, filtered and concentrated to give a crude product: the crude product was separated and purified through a flash column chromatography (EA/PE=1/3) to give compound 40-i (6.2 g, 75.7%) as a pale yellow liquid. LC-MS (ESI): m/z=274.1 [M+H]⁺.

### Synthesis of Compound 40-h

Compound 40-i (6.2 g, 22.7 mmol) was dissolved in DCM (100 mL): under nitrogen atmosphere. DMF-DMA (4.05 g, 34.1 mmol) was added at room temperature. The reaction mixture was stirred at room temperature for 45 minutes, and then BF₃.Et₂O (4.84 g, 34.1 mmol) was added. The mixture was stirred at room temperature for 1 hour and then diluted with dichloromethane (200 mL); the organic phase was washed successively with a saturated NaHCO₃ solution (400 mL) and saturated brine (200 mL), dried over anhydrous sodium sulfate, filtered and concentrated to give a crude product, which was separated and purified through a flash column chromatography (EA/PE=1/3) to give compound 40-h (5.3 g, 83%) as a pale red liquid. LC-MS (ESI): m/z=283.9 [M+H]⁺.

### Synthesis of Compound 40-g

At −78° C., under nitrogen atmosphere, to a solution of compound 40-h (5.3 g, 18.7 mmol) in THF (100 mL) was added dropwise tri-sec-butyl lithium borohydride tetrahydrofuran (1 M, 28.1 mL, 28.1 mmol). After completion of the addition, the mixture was stirred at this temperature for 1 hour: the reaction was quenched by adding saturated ammonium chloride (20 mL) and extracted with ethyl acetate (100 mL*3); the organic phase was washed with saturated sodium chloride and concentrated to give a crude product, which was separated and purified through a flash column chromatography (EA/PE=114) to give compound 40-g (5.3 g, 100%) as a yellow oil. LC-MS (ESI): m/z=286.2 [M+H]⁺.

### Synthesis of Compound 40-f

In an ice-water bath, to a solution of compound 40-g (5.3 g, 18.6 mmol) in methanol (150 mL) were successively added sodium methylate (10.0 g, 18.6 mmol) and 2-methyl-2-thiourea sulfate (10.6 g, 37.2 mmol). After completion of the addition, the reaction mixture was warmed to room temperature and stirred overnight. The pH was adjusted to 5 with a 1 M hydrochloric acid solution: the solid was precipitated out, filtered, washed with water (50 mL*3) and dried to give crude product 40-f (3.1 g, 51%) as a pale yellow solid. LC-MS (ESI): m/z=326.1 [M+H]⁺.

### Synthesis of Compound 40-e

In an ice-water bath, at 0° C., to a solution of compound 40-f (1.3 g, 4.0 mmol) in DCM (20 mL) and DMF (10 mL) was added thionyl chloride (2.5 mL); the mixture was stirred in an ice-water bath for 3 hours, quenched by adding ice water solution (80 mL) and extracted with DCM (80 mL*2); the organic phase was dried over anhydrous sodium sulfate, filtered and concentrated: the crude product was separated and purified though a flash column chromatography (EA/PE=1/25) to give compound 40-e (0.51 g, 37%). LC-MS (ESI): m/z=344.1 [M+H]⁺.

### Synthesis of Compound 40-d

At room temperature, compound 40-e (0.51 g, 1.49 mmol) was dissolved in DMF (12 mL), and DIPEA (0.39 g, 3.0 mmol) and benzyl (S)-2-cyanomethylpiperazine-1-carboxylate hydrochloride (0.48 g, 1.6 mmol) were successively added. After completion of the addition, under nitrogen protection, the mixture was stirred at 100° C. for 1 hour, cooled to room temperature, quenched by adding water (100 mL) and extracted with ethyl acetate (80 mL*2); the organic phase was washed with saturated brine (100 mL*3) and concentrated: the crude product was separated and purified through a flash column chromatography (EA/PE=1/1) to give compound 40-d (0.7 g, 83%) as a white solid. LC-MS (PSI): m/z=567.2 [M+H]⁺.

### Synthesis of Compound 40-c

Compound 40-d (0.7 g, 1.24 mmol) was dissolved in ethyl acetate (20 mL) and MCPBA (0.63 g, 3.1 mmol) was added at room temperature. The mixture was stirred at room temperature for 1 hour and then quenched by adding saturated sodium bicarbonate solution (50 mL), extracted with ethyl acetate (50 mL*2), filtered and concentrated to give crude product compound 40-c which was used directly in the next reaction.

### Synthesis of Compound 40-b

In an ice-water bath, to a solution of compound 40-c (0.66 g, 1.1 mmol) in toluene (10 mL) were successively added N-methyl-L-prolinol (0.254 g, 2.2 mmol) and t-BuONa (0.21 g, 2.2 mmol). After completion of the addition, under nitrogen atmosphere, the mixture was stirred in an ice-water bath for 0.5 hours and then quenched by adding water (10 mL) and extracted with ethyl acetate (30 mL*2); the organic phase was concentrated: the crude product was separated and purified through a flash column chromatography (MeOH/DCM=119) to give compound 40-b (0.39 g, 50% yield for two steps). LC-MS (ESI): m/z=634.0 [M+H]⁺.

### Synthesis of Compound 40-a

A solution of compound 40-b (0.18 g, 0.28 mmol) in methanolic ammonia (7 M, 50 mL) was cooled to −78° C., replaced twice with nitrogen, added 10% Pd—C (55 mg) and replaced three times with hydrogen. The reaction mixture was warmed to room temperature and stirred under hydrogen for 2 hours. The reaction mixture was filtered and concentrated to give compound 40-a (0.093 g, 66%). LC-MS (ESI): m/z=500.2 [M+H]⁺.

### Synthesis of Compound 40

At room temperature, compound 40-a (0.09 g, 0.18 mmol) was dissolved in DCM (10 mL), and DIPEA (0.046 g, 0.36 mmol) and acryloyl chloride (27 uL, 0.27 mmol) were successively added. Under nitrogen atmosphere, the mixture was stirred at room temperature overnight, quenched by adding water (10 mL) and extracted with DCM (50 mL*3); the organic phase was concentrated; the crude product was separated and purified through a flash column chromatography (MeOH/DCM=1/9) to give compound 40 (13 mg, 7%) as a white solid. LC-MS (ESI): m/z=554.3 [M+H]⁻; ¹H NMR (400 MHz, CD₃OD): δ 9.25 (s, 1H), 8.59 (d, 1H, J=6.8 Hz), 8.27 (d, 1H, J=8.8 Hz), 8.20 (d, 1H, J=7.6 Hz), 7.88 (t, 1H, J=7.6 Hz), 7.61 (t, 1H, J=7.6 Hz), 6.72-6.90 (m, 1H), 6.31 (d, 1H, J=15.6 Hz), 5.76-5.95 (m, 1H), 5.56-5.65 (m, 1H), 5.53 (t, 2H, J=10.0 Hz), 4.43-4.47 (m, 3H), 3.38-4.21 (m, 3H), 3.43-3.52 (m, 2H), 3.30-3.36 (m, 1H), 2.93-3.20 (m, 4H), 2.88-2.95 (m, 1H), 2.80-2.83 (m, 1H), 2.51-2.61 (m, 3H), 2.30-2.44 (m, 1H), 2.08-2.22 (m, 1H), 1.81-1.93 (m, 2H), 1.73-1.79 (m, 1H).

### Example 37 Synthetic Route of Compound 41

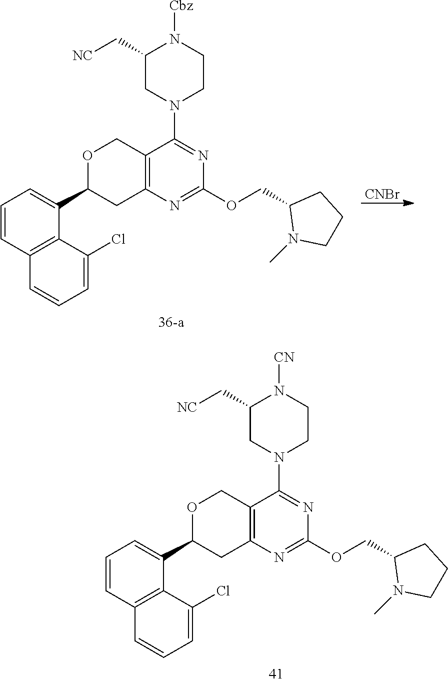

### Synthesis of Compound 41

In an ice bath, to a solution of compound 36-a (50 mg, 0.094 mmol) in DMF (2 mL) were added cesium carbonate (61.1 mg, 0.19 mmol) and cyanogen bromide (10.9 mg, 0.1 mmol). After completion of the addition, the reaction mixture was stirred at 0° C. for 2 hours. After completion of the reaction, the reaction mixture was filtered, and the filtrate was directly purified by Prep-HPLC to give compound 39 (5 mg, 9.6%) as a white solid. LC-MS (ESI): m/z=558.2 [M+H]⁺: ¹HNMR (400 MHz, CDCl₃): δ 7.95 (d, 1H, J=7.2 Hz), 7.83 (t, 2H, J=8.8 Hz), 7.60 (dd, 1H, J=7.6, 1.2 Hz), 7.56 (t, 1H, J=8.0 Hz), 7.37 (t, 1H, J=8.0 Hz), 6.44-6.54 (m, 1H), 4.87-4.96 (m, 1H), 4.72-4.80 (m, 1H), 4.36-4.44 (m, 1H), 4.13-4.21 (m, 1H), 3.91-4.00 (m, 1H), 3.75-3.88 (m, 1H), 3.46-3.67 (m, 4H), 3.25-3.44 (m, 2H), 3.06-3.16 (m, 1H), 2.81-2.95 (m, 3H), 2.62-2.74 (m, 1H), 2.48 (s, 3H), 2.21-2.35 (m, 1H), 1.96-2.11 (m, 1H), 1.66-1.92 (m, 3H).

### Example 38 Synthetic Route of Compound 42

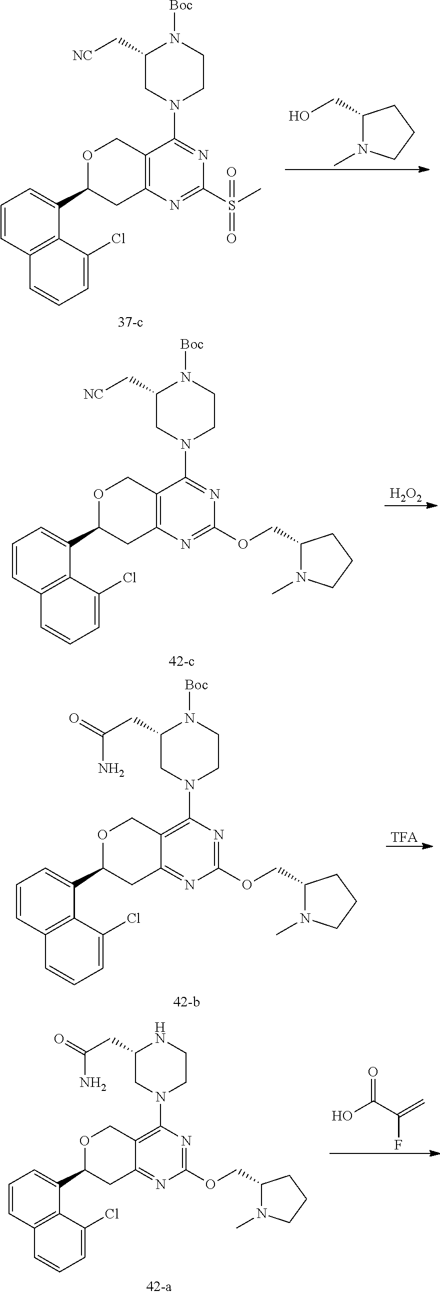

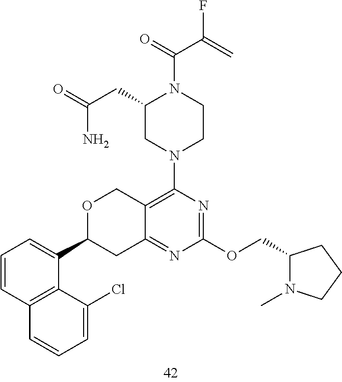 [SMILES: C=C(F)C(=O)N1CCN(c2nc(OC[C@@H]3CCCN3C)nc3c2CO[C@H](c2cccc4cccc(Cl)c24)C3)C[C@@H]1CC(N)=O]

### Synthesis of Compound 42-c

In an ice bath, to a solution of 37-c (600 mg, 1.0 mmol) and N-methyl-L-prolinol (231 mg, 2.0 mmol) in toluene (10 mL) was added sodium test-butoxide (192.8 mg, 2.0 mmol). After completion of the addition, the reaction mixture was stirred in an ice bath for 30 minutes. After completion of the addition, the reaction mixture was concentrated under reduced pressure, diluted with ethyl acetate, washed with water and saturated brine, dried over anhydrous sodium sulfate, filtered and subjected to rotary evaporation to give a brown oil, which was purified through a flash column chromatography to give 42-c (550 mg, 87%) as a white solid. LC-MS (ESI): m/z=633.2 [M+H]⁺.

### Synthesis of Compound 42-b

To a reaction flask were added compound 42-c (150 mg, 0.24 mmol), potassium carbonate (4.91 mg, 0.04 mmol) and dimethylsulfoxide (2 mL). In an ice bath, H₂O, in aqueous solution (30%, 107.4 mg, 0.95 mmol) was added dropwise: after completion of the addition, the mixture was warmed to room temperature and stirred overnight. The next day, the solid was precipitated out from the reaction mixture by adding water and then filtered: the filter cake was washed with water and dried to give 42-b (130 mg, 84%) as a white solid. LC-MS (ESI): m/z=651.3 [M+H]⁺.

### Synthesis of Compound 42-a

To a solution of 42-b (130 mg, 0.20 mmol) in dichloromethane (10 mL) was added trifluoroacetic acid (2 mL). The resulting reaction mixture was stirred at room temperature for 4 hours. After completion of the addition, the reaction mixture was concentrated, carefully neutralized to a pH of greater than 7 with saturated sodium bicarbonate solution in an ice bath and extracted with ethyl acetate: the organic layer was combined, washed with saturated brine, dried over anhydrous sodium sulfate, filtered and concentrated to give product 42-a (100 mg, 90%) as an amber-colored oil. LC-MS (ESI): m/z=551.2 [M+1]⁺.

### Synthesis of Compound 42

At room temperature, to a solution of 42-a (100 mg, 0.18 mmol) and HATU (138.0 mg, 0.36 mmol) in DMF (5 mL) were added N,N-diisopropylethylamine (70.4 mg, 0.54 mmol) and 2-fluoroacrylic acid (24.5 mg, 0.27 mmol). The reaction mixture was stirred at room temperature overnight. After completion of the reaction, the solid was precipitated out by slowly adding water, collected by filtration, washed with water and dried to give a crude product, which was then purified by Prep-HPLC to give 42 (60 mg, 53%) as a white solid. LC-MS (ESI): m/z=623.2 [M+H]⁺; ¹H NMR (400 MHz, CDCl₃): δ 7.95 (d, 1H, J-=6.8 Hz), 7.83 (t, 2H, J=8.4 Hz), 7.60 (dd, 1H, J=7.2, 1.2 Hz), 7.55 (t, 1H, J=7.6 Hz), 7.36 (t, 1H, J=8.0 Hz), 6.46 (dd, 1H, J=10.8, 3.2 Hz), 6.05-6.21 (m, 1H), 5.36 (s, 1H), 5.32 (dd, 1H, J=47.6, 4.0 Hz), 5.20 (dd, 1H, J=16.8, 3.6 Hz), 4.73-4.96 (m, 3H), 3.80-4.59 (m, 5H), 2.97-3.82 (m, 5H), 2.55 (s, 3H), 2.44-2.95 (m, 4H), 2.28-2.44 (m, 1H), 1.86-2.16 (m, 4H).

### Example 39 Synthetic Route of Compound 43

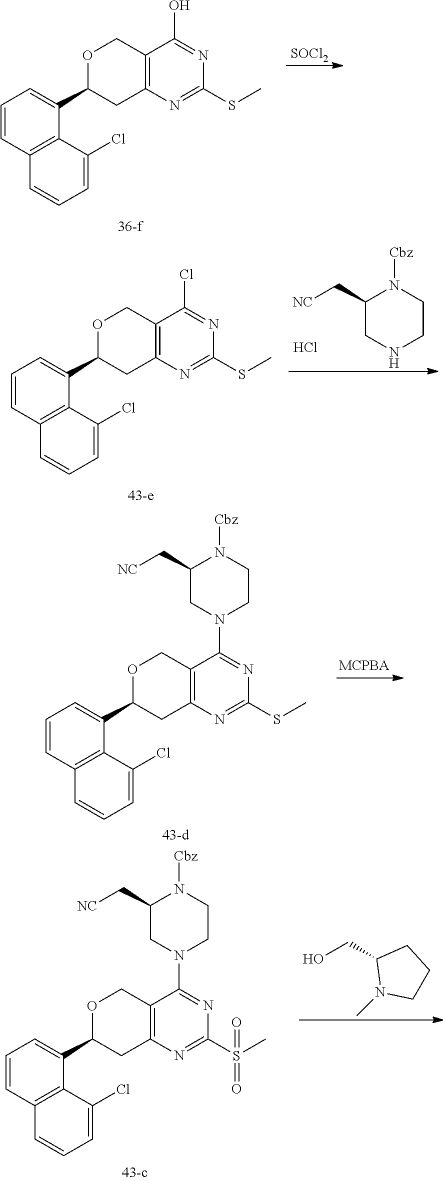

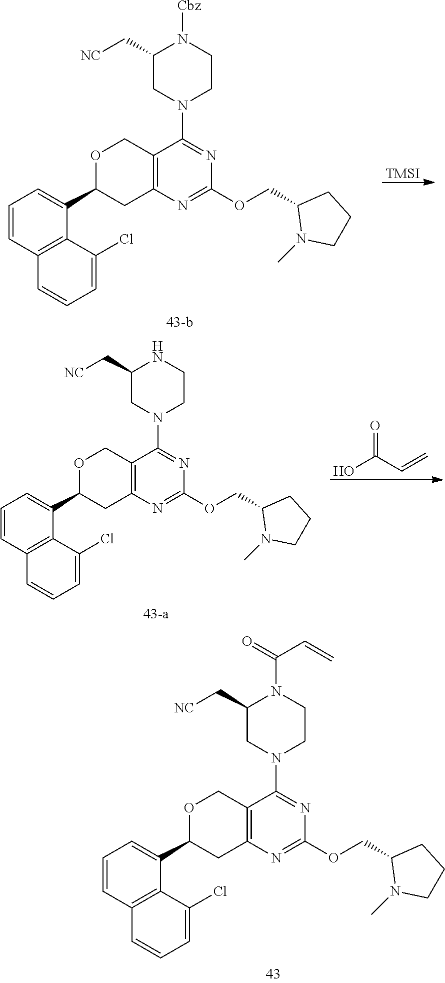

### Synthesis of Compound 43-e

In an ice-water bath, to a solution of 36-f (4.0 g, 11.1 mmol) in DMF (40 mL) and DCM (20 mL) was added dropwise thionyl chloride (9.3 g, 78.0 mmol). After completion of the addition, the reaction mixture was stirred in an ice-water bath for 4 hours. The reaction mixture was slowly added dropwise to 60 mL of water; the internal temperature was controlled at 0° C., to 10° C., and the reaction mixture was extracted with DCM. The organic phase was washed with saturated sodium bicarbonate and water, concentrated, slurred with n-heptane, cooled to 0° C., to 10° C. filtered and dried to give compound 43-e (3.2 g, 76%). LC-MS (ESI): m/z=377.0 [M+1]⁺. The structure of this compound was confirmed by single crystal analysis.

### Synthesis of Compound 43-d

At room temperature, to a solution of 43-e (150 mg, 0.40 mmol) in DMF (10 mL) were respectively added DIPEA (154.2 mg, 1.19 mmol) and benzyl (R)-2-cyanomethylpiperazine-1-carboxylate hydrochloride (134 mg, 0.52 mmol). After completion of the addition, the reaction mixture was heated to 100° C., and stirred for two hours. After completion of the reaction, the reaction mixture was diluted by adding ethyl acetate, washed successively with water and saturated brine, dried over anhydrous sodium sulfate, filtered and subjected to rotary evaporation. The crude product was separated and purified through a flash column chromatography to give 43-d (0.2 g, 84%) as a white solid. LC-MS (ESI): m/z=600.0 [M+H]⁺.

### Synthesis of Compound 43-c

In an ice bath, compound 43-d (0.2 g, 0.33 mmol) was dissolved in ethyl acetate (20 mL) and then added m-chloroperoxybenzoic acid (143.8 mg, 0.83 mmol); the mixture was slowly warmed to room temperature and stirred for 2 hours. After completion of the reaction, the reaction mixture was neutralized by adding saturated aqueous sodium bicarbonate solution and extracted with ethyl acetate (15 mL*2); the organic phase was combined, dried over anhydrous sodium sulfate, filtered and concentrated: the crude product was purified through a flash column chromatography (PE/EA=2/1) to give 43-c (0.18 g, 85%) as a white solid. LC-MS (ESI): m/z=632.0 [M+1]⁺.

### Synthesis of Compound 43-b

In an ice bath, to a solution of 43-c (180 mg, 0.28 mmol) and N-methyl-L-prolinol (65.6 mg, 0.57 mmol) in toluene (10 mL) was added sodium tert-butoxide (54.7 mg, 0.57 mmol). After completion of the addition, the reaction mixture was stirred in an ice bath for 10 minutes. After completion of the addition, the reaction mixture was concentrated under reduced pressure, diluted with ethyl acetate, washed with water and saturated brine, dried over anhydrous sodium sulfate, filtered and subjected to rotary evaporation to give a brown oil, which was purified through a flash column chromatography to give 43-b (150 mg, 79%) as a white solid. LC-MS (ESI): m/z=667.2 [M+H]⁺.

### Synthesis of Compound 43-a

At room temperature, to a solution of 43-b (150 mg, 0.22 mmol) un acetonitrile (20 mL) was added trimethyliodosilane (224.9 mg, 1.12 mmol). After completion of the addition, the reaction mixture was heated to 30° C., and stirred for 5 hours. After completion of the addition, the reactant was neutralized with triethylamine (5 mL) and concentrated under reduced pressure to give 43-a (80 mg, 67%) as a dark grey solid. LC-MS (ESI): m/z=533.2 [M+H]⁺.

### Synthesis of Compound 43

At room temperature, to a solution of 43-a (80 mg, 0.15 mmol) and HATU (114.1 mg, 0.30 mmol) in DMF (5 mL) were added N,N-diisopropylethylamine (58.2 mg, 0.45 mmol) and acyclic acid (16.2 mg, 0.23 mmol), respectively. The reaction mixture was stirred at room temperature overnight. After completion of the reaction, the solid was precipitated out by slowly adding water, collected by filtration, washed with water and dried to give a crude product, which was then purified by silica gel column chromatography (DCM/MeOH 10/1) to give 43 (40 mg, 45%) as a white solid. LC-MS (ESI): m/z=587.3 [M+H]⁺: ¹H NMR (400 MHz, CDCl₃): δ 7.96 (d, 1H, J=7.2 Hz), 7.84 (t, 2H, J=9.2 Hz), 7.61 (d, 1H, J=7.2 Hz), 7.56 (t, 1H, J=8.0 Hz), 7.38 (t, 1H, J=8.0 Hz), 6.53-6.75 (m, 1H), 6.46-6.53 (m, 1H), 6.36 (d, 1H, J=16.4 Hz), 5.83 (d, 1H, J=10.4 Hz), 4.82-5.03 (m, 3H), 3.74-4.75 (m, 5H), 3.23-3.71 (m, 6H), 3.02-3.19 (m, 2H), 2.95 (s, 3H), 2.68-2.90 (m, 2H), 2.01-2.30 (m, 4H).

### Example 40 Synthetic Route of Compound 44

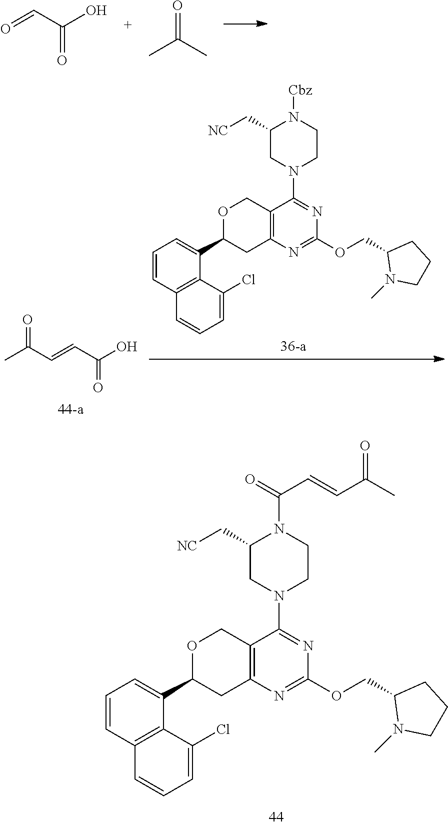

### Synthesis of Compound 44-a

At room temperature, to a solution of compound glyoxylic acid (50% in H₂O, 2 g, 13.5 mmol) in acetone (20 mL) was added morpholine hydrochloride (1.67 g, 13.5 mmol). The reaction mixture was stirred at room temperature for 1 hour and then heated to reflux overnight. After completion of the addition, acetone was removed by concentration. The crude product was diluted with water and extracted with ethyl acetate. The organic phase was dried over anhydrous sodium sulfate, filtered and concentrated to give compound 44-a (0.5 g, 32%) as a white solid. LC-MS (ESI): m/z=115.1 [M+H]⁺: ¹H NMR (400 MHz, DMSO-d₆): δ 13.12 (bs, 1H), 6.80 (d, 1H, J=16 Hz), 6.66 (d, 1H, J=16 Hz), 2.34 (s, 3H).

### Synthesis of Compound 44

To a solution of 36-a (50 mg, 0.094 mmol) and HATU (71.3 mg, 0.19 mmol) in DMF (5 mL) were added N,N-diisopropylethyl amine (36.4 mg, 0.28 mmol) and 44-a (10.1 mg, 0.14 mmol) respectively. The reaction mixture was stirred at room temperature overnight. After completion of the reaction, the solid was precipitated out by slowly adding water, collected by filtration, washed with water and dried to give a crude product, which was then purified by silica gel column chromatography (DCM/MeOH 101) to give compound 44 (25 mg, 42%) as a white solid. LC-MS (ESI): m/z=629.2 [M+H]⁺; ¹H NMR (400 MHz, CDCl₃): δ 7.97 (d, 1H, J=6.8 Hz), 7.83 (t, 2H, J=8.8 Hz), 7.60 (d, 1H, J=7.2 Hz), 7.56 (t, 1H, J=4.0 Hz), 7.36 (t, 1H, J=7.6 Hz), 7.07-7.17 (m, 1H), 6.49-6.53 (m, 1H), 4.51-5.14 (m, 3H), 4.44 (dd, 1H, J=10.4, 5.2 Hz), 4.18 (dd, 1H, J=10.8, 2.4 Hz), 3.36-4.02 (m, 5H), 2.64-3.30 (m, 7H), 2.51 (s, 3H), 2.38 (s, 3H), 2.27-2.63 (m, 2H), 2.00-2.13 (m, 1H), 1.70-1.94 (m, 3H).

### Example 41 Synthetic Route of Compound 45

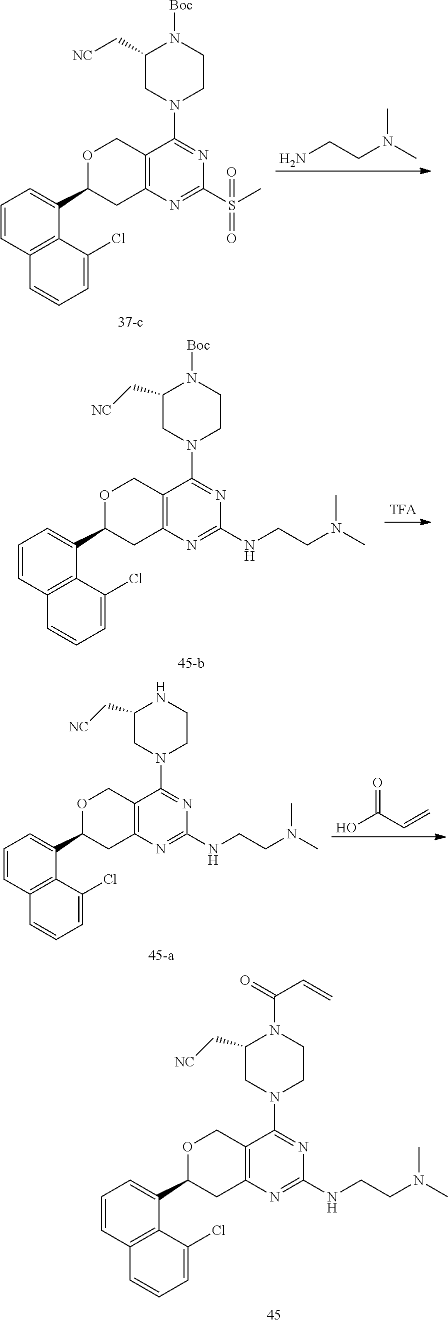

### Synthesis of Compound 45-b

At room temperature, to a solution of 37-c (150 mg, 0.25 mmol) in dioxane (20 mL) was added N,N-methylethylenediamine (110.5 mg, 1.25 mmol). The reaction mixture was heated to reflux overnight. After completion of the addition, the reaction mixture was concentrated under reduced pressure, diluted with ethyl acetate, washed with water and saturated brine, dried over anhydrous sodium sulfate, filtered and subjected to rotary evaporation to give a brown oil, which was purified through a flash column chromatography to give 45-b (130 mg, 85%) as a white solid. LC-MS (ESI): m/z=606.2 [M+H]⁺.

### Synthesis of Compound 45-a

To a solution of 45-b (130 mg, 0.21 mmol) in dichloromethane (10 mL) was added trifluoroacetic acid (2 mL). The resulting reaction mixture was stirred at room temperature for 4 hours. After completion of the addition, the reaction mixture was concentrated, carefully neutralized to a pH of greater than 7 with saturated sodium bicarbonate solution in an ice bath and extracted with ethyl acetate; the organic layer was combined, washed with saturated brine, dried over anhydrous sodium sulfate, filtered and concentrated to give product 45-a (80 mg, 74%) as an amber-colored oil. LC-MS (ESI): m/z=506.2 [M+1]⁺.

### Synthesis of Compound 45

At room temperature, to a solution of 45-a (80 mg, 0.16 mmol) and HATU (120.2 mg, 0.32 mmol) in DMF (5 mL) were added DIPEA (61.3 mg, 0.48 mmol) and acyclic acid (17.1 mg, 0.24 mmol), respectively. The reaction mixture was stirred at room temperature overnight. After completion of the reaction, the solid was precipitated out by slowly adding water, collected by filtration, washed with water and dried to give a crude product, which was then purified by silica gel column chromatography (DCM/MeOH 10/1) to give 45 (15 mg, 17%) as a white solid. LC-MS (ESI): m/z=560.2 [M+H]⁺: ¹H NMR (400 MHz, CDCl₃); δ 7.98 (d 1H, J=7.6 Hz), 7.82 (t, 2H, J=7.6 Hz), 7.52-7.61 (m, 2H), 7.36 (t, 1H, J=7.6 Hz), 6.52-6.66 (m, 1H), 6.48 (dd, 1H, J=10.8, 3.2 Hz), 6.38 (d, 1H, J=16.4 Hz), 5.77-5.86 (m, 1H), 5.42-5.55 (m, 1H), 4.47-5.15 (m, 3H), 3.25-4.06 (m, 8H), 2.89-3.09 (m, 1H), 2.59-2.88 (m, 5H), 2.41 (s, 6H).

### Example 42 Synthetic Route of Compound 46

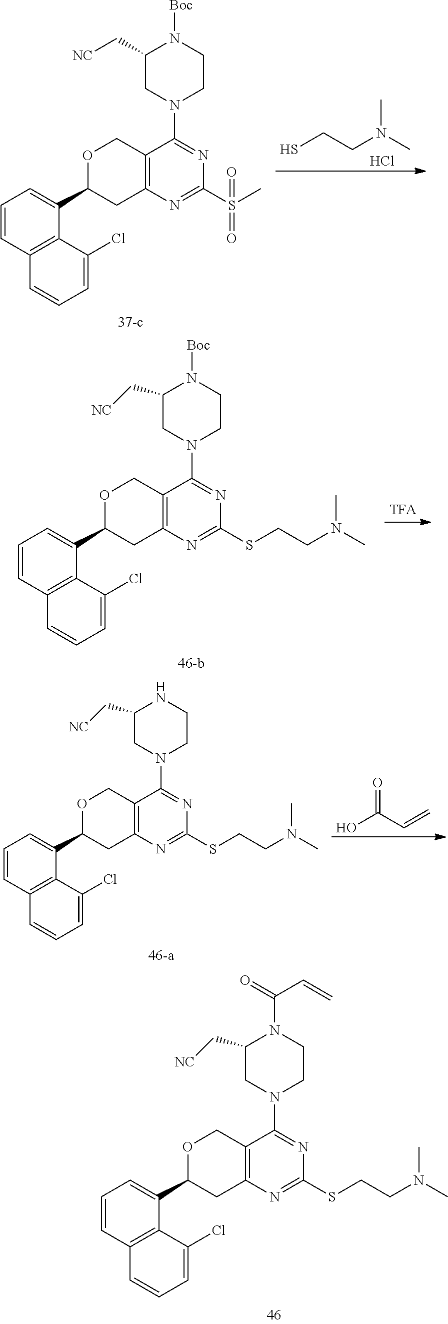

### Synthesis of Compound 46-b

At room temperature, to a solution of 37-c (200 mg, 0.33 mmol) in acetonitrile (20 mL) were added 2-dimethylaminoethanethiol hydrochloride (236.8 mg, 1.67 mmol) and triethylamine (338.4 mg, 3.34 mmol). After completion of the addition, the reaction mixture was heated to reflux overnight. After completion of the addition, the reaction mixture was concentrated under reduced pressure, diluted with ethyl acetate, washed with water and saturated brine, dried over anhydrous sodium sulfate, filtered and subjected to rotary evaporation to give a brown oil, which was purified through a flash column chromatography to give 46-b (100 mg, 48%) as a white solid. LC-MS (ESI): m/z=623.2 [M+H]⁺.

### Synthesis of Compound 46-a

To a solution of 46-b (100 mg, 0.16 mmol) in dichloromethane (10 mL) was added trifluoroacetic acid (2 mL). The resulting reaction mixture was stirred at room temperature for 4 hours. After completion of the addition, the reaction mixture was concentrated, carefully neutralized to a pH of greater than 7 with saturated sodium bicarbonate solution in an ice bath and extracted with ethyl acetate; the organic layer was combined, washed with saturated brine, dried over anhydrous sodium sulfate, filtered and concentrated to give product 46-a (70 mg, 83%) as an amber-colored oil. LC-MS (ESI): m/z=523.0 [M+H]⁺.

### Synthesis of Compound 46

At room temperature, to a solution of 46-a (70 mg, 0.16 mmol) and HATU (101.8 tag, 0.27 mmol) in DMF (5 mL) were added DIPEA (51.9 mg, 0.40 mmol) and acyclic acid (14.5 mg, 0.20 mmol). The reaction mixture was stirred at room temperature overnight. After completion of the reaction, the solid was precipitated out by slowly adding water, collected by filtration, washed with water and dried to give a crude product, which was then purified by silica gel column chromatography (DCM/MeOH 10/1) to give 46 (15 mg, 19%) as a white solid. LC-MS (ESI): m/z=577.2 [M+H]⁺: ¹H NMR (400 MHz, DMSO-d₆); δ 7.95-8.04 (m, 3H), 7.65-7.74 (m, 2H), 7.46-7.55 (m, 1H), 6.78-6.98 (m, 1H), 6.34-6.47 (m, 1H), 5.76-6.21 (m, 1H), 4.54-5.16 (m, 3H), 3.78-4.49 (m, 3H), 3.37-3.77 (m, 6H), 3.02-3.28 (m, 6H), 2.61-3.04 (m, 7H), 2.26-2.43 (m, 1H).

### Example 43 Synthesis of Comparative Compound 3′

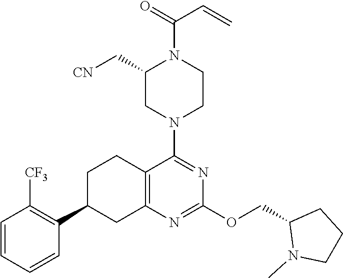

Comparative compound 3′ was synthesized with reference to the method in patent WO 2019155399 A1.

### Effect Example 1 Experiments for Testing Proliferation Inhibition of Compounds on NCI-H358, A549 and A375 Cell Lines by CTG Method

NCI-H358 was a human non-small cell lung cancer cell with KRAS G12C mutation; A549 was a human non-small cell lung cancer cell with KRAS G12V mutation: and A375 was a wild-type malignant melanoma cell. The inhibitory effects of compounds on different mutations were evaluated by testing the proliferation inhibitory activity of these compounds on the three cell lines.

40 μL of cell suspension to be tested was added to each well (except for peripheral wells) of three 384-well plates (plate 1: NCI-H358 cell suspension: plate 2: A549 cell suspension: plate 3: A375 cell suspension). The plates were placed in a carbon dioxide incubator overnight. The prepared compounds (3-fold dilutions were made to give 10 concentration gradients of the compounds) were added to each well. The plates were incubated in a carbon dioxide incubator for 120 hours. To the 384-well plates were added 25 μL of CellTiter Glo reagents, and the mixture was shaken in the dark for 10 minutes and incubated for 10 minutes. The plates were read on the EnVision plate reader. XLFit was used to draw inhibition rate curves of efficacy and IC₅₀ values were calculated. Table 1 shows the activity results of representative compounds. “*” represents “IC₅₀>1 μM” and “**” represents “IC₅₀≤1 uM”. “---” represents not determined.

TABLE 1
Proliferation inhibitory activity of representative compounds of
the present disclosure on H358 cells, A549 cells and A375 cells

| | IC₅₀ (μM): | IC₅₀ (μM): | IC₅₀ (μM): |
|---|---|---|---|
| Compound No. | (H358) | (A549) | (A375) |
| 1 | * (>10) | * (>10) | — |
| 2 | ** | * | — |
| 2-1 | ** | * | * |
| 2-2 | ** (0.0025) | * | * |
| 3 | ** | * | — |
| 3-1 | ** | * | * |
| 3-2 | ** | * | * |
| 4 | ** | * | — |
| 4-1 | ** | * | * |
| 4-2 | ** | * | * |
| 5 | ** | * | — |
| 6-2 | ** | * | — |
| 7 | ** | * | — |
| 8 | ** | * | — |
| 9 | ** | * | — |
| 10 | ** | * | * |
| 11-2 | ** | * | * |
| 12 | ** | * | * |
| 13 | ** | * | * |
| 12-2 | ** | * | * |
| 13-2 | ** | * | * |
| 14 | ** | * | * |
| 15 | ** | * | * |
| 16 | ** | * | * |
| 17 | ** | * | * |
| 18-2 | ** (0.013) | * | * |
| 19-2 | ** | * | * |
| 24 | ** | * | * |
| 25 | ** | * | * |
| 30 | ** | * | * |
| 31 | ** | * | * |
| 32-2 | ** | * | * |
| Comparative compound 1′ | 0.0073 | — | — |
| Comparative compound 2′ | 0.0394 | — | — |

It can be determined from the results of the above-mentioned tests that the compounds of the present disclosure had significant proliferation inhibitory effects on NCI-H358 cells, but had weak proliferation inhibitory activities on A549 cells and A375 cells.

In addition, pyranopyrimidine backbone compounds 2-2 and 18-2 of the present disclosure and the corresponding piperidinopyrimidine backbone compounds, comparative compound and comparative compound 2′, were compared in terms of activities. It can be seen from the results that the activity of compound 2-2 was significantly better than that of the corresponding comparative compound 1′, and the activity of compound 18-2 was significantly better than that of the corresponding comparative compound 2′.

### Effect Example 2

Experiments for Testing Proliferation Inhibition of Compounds on NCI-H358 Cell Lines by CTG Method

The operation was the same as that in effect example 1

Experiments for Testing Proliferation Inhibition of Compounds on MIA PaCa-2 Cell Lines by CTG Method

40 μL of MIA PaCa-2 cell suspension was added to each well (except for peripheral wells) of 384-well plates. The plates were placed in a carbon dioxide incubator overnight. The prepared compounds (3-fold dilutions were made to give 10 concentration gradients of the compounds) were added to each well. The plates were incubated in a carbon dioxide incubator for 120 hours. To the 384-well plates were added 25 μL of CellTiter Glo reagents, and the mixture was shaken in the dark for 10 minutes and incubated for 10 minutes. The plates were read on the EnVision plate reader. XLFit was used to draw inhibition rate curves of efficacy and IC₅₀ values were calculated. Table 1 shows the activity results of representative compounds. “*” represents “IC₅₀>1 μM” and “**” represents “IC₅₀≤1 μM”. “---” represents not determined.

MIA PaCa-2 was a human pancreatic cancer cell with KRAS G12C mutation. The activity of compounds can be evaluated by detecting the proliferation inhibitory activity of these compounds on such cell line.

TABLE 2
Proliferation inhibitory activity of representative compounds
of the present disclosure on H358 and MIA PaCa-2 cells

| | IC₅₀ (nM) | IC₅₀ (nM) |
|---|---|---|
| Compound No. | H358 | MIA PaCa-2 |
| 2-2 | 2.5 | 1.9 |
| Comparative compound 1′ | 7.3 | 3.9 |
| Comparative compound 3′ | — | 8.3 |
| 18-2 | 13.0 | 9.9 |
| Comparative compound 2′ | 39.4 | 36.4 |
| 35 | — | ** |
| 36 | ** | — |
| 37 | ** | — |
| 38 | — | ** |
| 40 | — | ** |
| 41 | ** | — |
| 43 | ** | — |
| 44 | ** | — |
| 45 | ** | — |
| 46 | ** | — |

It can be seen from the results that the activity of compound 2-2 was significantly better than that of the corresponding comparative compounds 1′ and 3′, and the activity of compound 18-2 was significantly better than that of the corresponding comparative compound 2′.

### Effect Example 3 Inhibition Activity of Metabolic Enzymes In Vitro

Test purpose: In this test, human liver microsomes and CYP enzyme-specific substrates were used to test the effects of different concentrations of compounds to be tested on metabolic rates of enzyme-specific substrates, so as to determine inhibitory effects of these compounds on CYP enzymes in human liver microsomes.

Operation Procedure:

1. preparing 100 nM potassium phosphate buffer (pH 7.4);

2. preparing stock solutions of compounds to be tested (10 mM), NADPH solutions (8 mM, which was 4 times the final concentration), positive compound stock solutions, substrate stock solutions and human liver microsome solutions (see the table below);

3. preparing solutions of compounds to be tested: dissolving 8 μL of 10 mM stock solution of compounds to be tested in 12 μL of acetonitrile, and then using a mixed solution of 40% DMSO and 60% acetonitrile to perform a 1:3 gradient dilution as follows (wherein the concentration was 400 times the final concentration): 4 mM, 1.333 mM, 0.444 mM, 0.148 mM, 0.0494 mM, 0.0165 mM, 0.00549 mM and 0 mM.

4. preparing positive control inhibitor solutions: dissolving 8 μL of positive control inhibitor stock solutions in 12 μL of acetonitrile, and then using a mixed solution of 40% DMSO and 60% acetonitrile to perform a 1:3 gradient dilution as follows (wherein the concentration was 400 times the final concentration, which was described in the table below);

5. preparing mixed solutions of compounds to be tested and human liver microsomes: adding 400 μL of 0.2 mg/mL human liver microsomes to a 96-well plate, and adding 2 μL of solutions of compounds to be tested at a concentration of 400-times higher than that of the microsomes;

6. preparing mixed solutions of positive control compounds and human liver microsomes: adding 200 μL of 0.2 mg/mL human liver microsomes to a 96-well plate, and adding 1 μL of diluted solutions of positive control compounds;

7. dispensing 30 μL of mixed solutions of the compounds and human liver microsomes to a 96-well plate, and then adding 15 of substrate solutions: adding 15 of preheated NADPH solutions to a preheated reaction plate and mixing evenly to start the reaction;

8. incubating the reaction plate at 37° C.; 3A4 was reacted for 5 minutes; 1A2, 2C₉ and 2D6 were reacted for 10 minutes; 2C19 was reacted for 45 minutes;

9, after completion of the addition, adding 120 μL of acetonitrile containing internal standard to terminate the reaction: after terminating the reaction, shaking the sample on a shaker at 600 rpm/min for 10 minutes and centrifuging same at 3220×g for 15 minutes; after centrifugation, taking out 50 μL of supernatant and mixing same with 50 μL of water for analysis by LC-MS/MS.

| | | | | | | Initial | Human liver | |
|---|---|---|---|---|---|---|---|---|
| | | Concentration | Final | | Concentration | final | microsome | Incubation |
| CYP | | of stock | concentration | Positive | of stock | concentration | concentration | time |
| enzyme | Substrate | solution | (μM) | compound | solution | (μM) | (mg/mL) | (min) |
| 1A2 | Phenacetin | 6 mM | 30 | α-naphthoflavone | 0.3 mM | 0.3 | 0.1 | 10 |
| | | in ACN | | | in DMSO | | | |
| 2C9 | Diclofenac | 10 mM | 10 | Sulfaphenazole | 10 mM | 10 | 0.1 | 10 |
| | | in H₂O | | | in DMSO | | | |
| 2C19 | S-Mephenytoin | 35 mM | 35 | Omeprazole | 100 mM | 100 | 0.5 | 45 |
| | | in ACN | | | in DMSO | | | |
| 2D6 | Bufuralol | 10 mM | 10 | Quinidine | 2.5 mM | 2.5 | 0.1 | 10 |
| | | in H₂O | | | in DMSO | | | |
| 3A4 | Testosterone | 10 mM | 80 | Ketoconazole | 2.5 mM | 2.5 | 0.1 | 5 |
| | | in ACN | | | in DMSO | | | |
| | Midazolam | 1 mM | 5 | | | | | |
| | | in ACN | | | | | | |

Data Processing:

According to the following equation, the data was plotted and fitted using a dose-response model in GraphPad Prism software to calculate IC₅₀.

Y=Bottom+(Top−Bottom)/(1+10\hat{ }((Log IC₅₀−X)×HillSlope))

X represents the logarithm concentration of the inhibitor, and Y represents the relative activity of the enzyme at a certain inhibitor concentration (relative to the condition without inhibitor).

Test Results:

| | IC₅₀ (μM) | | | | | |
|---|---|---|---|---|---|---|
| | | | | | CYP3A4 | CYP3A4 |
| Compound | CYP1A2 | CYP2C9 | CYP2C19 | CYP2D6 | (Midazolam) | (Testosterone) |
| 18-2 | >10 | >10 | >10 | >10 | >10 | >10 |
| MRTX849 | >10 | 2.29 | >10 | >10 | 5.92 | 7.85 |
| AMG-510 | >10 | >10 | >10 | >10 | 4.46 | >10 |

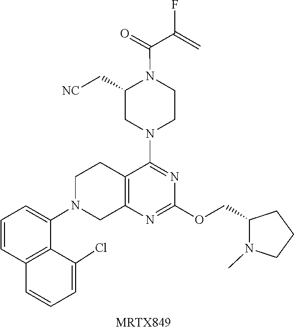

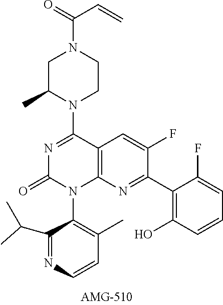

The results show that the inhibition IC₅₀ of 18-2 to CYP isoenzymes (1A2, 2C9, 2C19, 2D6 and 3A4) is >10, >10, >10, >10 and >10, respectively, indicating that this compound has a weak inhibition on all metabolic enzymes (IC₅₀>10 μmol/L) and was better than the current clinical compounds MRTX849 and AMG-510.

Although the specific embodiments of the present disclosure have been described above, it will be understood by those of skill in the art that these are merely illustrative, and that various alterations or modifications can be made to these embodiments without departing from the principle and essence of the present disclosure. Therefore, the scope of protection of the present disclosure is defined by the appended claims.

1. An oxygen-containing heterocyclic compound represented by formula I, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein: 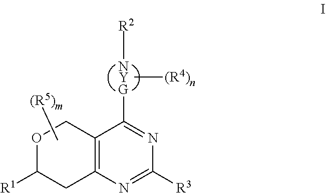
R¹ is C₆₋₂₀ aryl, “5-12 membered heteroaryl containing 1-4 heteroatoms selected from one or more of O, S and N”, C₆₋₂₀ aryl substituted with one or more R¹⁻⁶, or “5-12 membered heteroaryl containing 1-4 heteroatoms selected from one or more of O, S and N” substituted with one or more R¹⁻⁷;
R¹⁻⁶ and R¹⁻⁷ are independently halogen, hydroxyl, —C(═O)R⁶⁵, —NR⁶³R⁶⁴, —C(═O)OR⁶⁶, —C(═O)NR⁶⁹R⁶¹⁰, C₁₋₆ alkyl, C₁₋₆ alkoxy, C₃₋₁₀ cycloalkyl, “5-7 membered heterocycloalkyl containing 1 or 2 heteroatoms selected from one or more of O and N”, C₆₋₂₀ aryl, “5-7 membered heteroaryl containing 1 or 2 heteroatoms selected from one or more of O and N”, C₁₋₆ alkyl substituted with one or more R¹⁻⁶⁻¹ C₁₋₆ alkoxy substituted with one or more R¹⁻⁶⁻², C₃₋₁₀ cycloalkyl substituted with one or more R¹⁻⁶⁻³, “5-7 membered heterocycloalkyl containing 1 or 2 heteroatoms selected from one or more of O and N” substituted with one or more R¹⁻⁶⁻¹, C₆₋₂₀ aryl substituted with one or more R¹⁻⁶⁻⁵, or “5-7 membered heteroaryl containing 1 or 2 heteroatoms selected from one or more of O and N” substituted with one or more R¹⁻⁶⁻⁶;
R¹⁻⁶⁻¹, R¹⁻⁶⁻², R¹⁻⁶⁻³, R¹⁻⁶⁻⁴, R¹⁻⁶⁻⁵ and R¹⁻⁶⁻⁶ are independently cyano, halogen, hydroxyl, C₁₋₆ alkoxy, C₁₋₆ alkyl, —C(═O)R⁶⁵⁻², —NR⁶³⁻², —C(═O)OR⁶⁶⁻², or —C(═O)NR⁶⁹⁻²R⁶¹⁰⁻²;
R⁶⁵, R⁶⁵⁻², R⁶³, R⁶³⁻², R⁶⁴, R⁶⁴⁻², R⁶⁶, R⁶⁶⁻², R⁶⁹, R⁶⁹⁻², R⁶¹⁰ and R⁶¹⁰⁻² are independently hydrogen or C₁₋₆ alkyl;
m is 0, 1 or 2;
R⁵ is independently C₁₋₆ alkyl;
R³ is —OR³¹, —SR³² or —NR³³R³⁴;
R³¹, R³² and R³⁴ are independently C₁₋₆ alkyl substituted with one or more R³¹⁻¹; R³³ is independently H, C₁₋₆ alky, or C₁₋₆ alkyl substituted with one or more R³¹⁻¹ is independently C₃₋₁₀ cycloalkyl, “4-10 membered heterocycloalkyl containing 1-3 heteroatoms selected from one or more of O and N”, C₃₋₁₀ cycloalkyl substituted with one or more R^{d15}, “4-10 membered heterocycloalkyl containing 1-3 heteroatoms selected from one or more of O and N” substituted with one or more R^{d15}, —OR^{d}, —SR^{d1}, —NR^{e1}R^{e2}, or —C(═O)NR^{e3}R^{e4};
R^{d15} and R^{d16} are independently C₁₋₆ alkyl, C₁₋₆ alkyl substituted with one or more R¹⁻⁸⁻¹, hydroxyl, C₁₋₆ alkoxy, halogen, —NR^{e5}R^{e6} or —C(═O)NR^{e7}R^{e8};
R^{d}, R^{d1}, R^{e1}, R^{e2}, R^{e3} and R^{e4} are independently hydrogen, C₁₋₆ alkyl, C₃₋₁₀ cycloalkyl, “4-10 membered heterocycloalkyl containing 1-3 heteroatoms selected from one or more of O and N”, or C₁₋₆ alkyl substituted with one or more R¹⁻⁸⁻²;
R¹⁻⁸⁻¹ and R¹⁻⁸⁻² are independently cyano, halogen, hydroxyl, C₁₋₆ alkoxy, —C(═O)R^{e9}, —NR^{e10}R^{e11}, —C(═O)OR^{e12}, or —C(═O)NR^{e13}R^{e14};
R^{e5}, R^{e6}, R^{e7}, R^{e8}, R^{e9}, R^{e10}, R^{e11}, R^{e12}, R^{e13} and R^{e14} are independently hydrogen or C₁₋₆ alkyl;
ring Y is a 4-12 membered heterocyclic ring containing 1-4 N atoms; the heterocyclic ring is a saturated heterocyclic ring or a partly saturated heterocyclic ring; the heterocyclic ring is a monocyclic ring, a bridged ring or a spiro ring;
G is N, C or CH;
n is 0, 1, 2 or 3;
R⁴ is independently C₁₋₆ alkyl, C₁₋₆ alkyl substituted with one or more R⁴⁻¹, oxo, —C(═O)OR^{4a} or —C(═O)NR^{4b}R^{4c};
R⁴⁻¹ is independently halogen, cyano, hydroxyl, C₁₋₆ alkoxy, —NR⁴ⁱR^{4j}, —C(═O)OR^{4d} or —C(═O)NR^{4e}R^{4f}; R^{4d}, R^{4e}, R^{4f}, R⁴ⁱ and R^{4j} are independently hydrogen or C₁₋₆ alkyl;
R^{4a}, R^{4b} and R^{4c} are independently hydrogen or C₁₋₆ alkyl;
R² is CN, —C(═O)—C(R^{a})═C(R^{b})(R^{f}), —C(═O)—C≡CR^{f}, —S(═O)₂—C(R^{a})═C(R^{b})(R^{f}) or —S(═O)₂—C≡CR^{f};
R^{a} is independently hydrogen, deuterium, halogen or C₁₋₆ alkyl;
R^{b} and R^{f} are independently hydrogen, deuterium, C₁₋₆ alkyl, C₁₋₆ alkyl-C(═O)—, or C₁₋₆ alkyl substituted with one or more R^{b-1};
R^{b-1} is independently halogen, hydroxyl, C₁₋₆ alkoxy, or —NR^{10j}R^{10k};
R^{10j} and R^{10k} are independently hydrogen or C₁₋₆ alkyl, or R^{10j} and R^{10k} taken together with the N atom to which they are attached form “4-10 membered heterocycloalkyl containing 1-3 heteroatoms selected from one or more of 0 and N”.

2. The oxygen-containing heterocyclic compound represented by formula I as defined in claim 1, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein, R¹ is C₆₋₂₀ aryl, “5-12 membered heteroaryl containing 1-4 heteroatoms selected from one or more of O, S and N”, C₆₋₂₀ aryl substituted with one or more R¹⁻⁶, or “5-12 membered heteroaryl containing 1-4 heteroatoms selected from one or more of O, S and N” substituted with one or more R¹⁻⁷; R¹⁻⁶ and R¹⁻⁷ are independently halogen, hydroxyl, C₁₋₆ alkyl, C₁₋₆ alkoxy, C₃₋₁₀ cycloalkyl, C₁₋₆ alkyl substituted with one or more R¹⁻⁶⁻¹, or C₁₋₆ alkoxy substituted with one or more R¹⁻⁶⁻²; R¹⁻⁶⁻¹ and R¹⁻⁶⁻² are independently halogen;
and/or, m is 0;
and/or, R³ is —OR³¹ or —NR³³R³⁴; R³¹, R³³ and R³⁴ are independently C₁₋₆ alkyl substituted with one or more R³¹⁻¹; R³¹⁻¹ is independently “5-7 membered heterocycloalkyl containing 1-3 heteroatoms selected from one or more of O and N” substituted with one or more R^{d15}, or —NR^{e1}R^{e2}; R^{d15} is independently C₁₋₆ alkyl, C₁₋₆ alkyl substituted with one or more R¹⁻⁸⁻¹, hydroxyl, C₁₋₆ alkoxy, halogen, —NR^{e5}R^{e6} or —C(═O)NR^{e7}R^{e8}; R¹⁻⁸⁻¹ is independently halogen; R^{e5}, R^{e6}, R^{e7} and R^{e8} are independently hydrogen or C₁₋₆ alkyl: R^{e1} and R^{e2} are independently C₁₋₆ alkyl;
and/or, ring Y is a 4-12 membered heterocyclic ring containing 1-4 N atoms; the heterocyclic ring is a saturated heterocyclic ring or a partly saturated heterocyclic ring; the heterocyclic ring is a monocyclic ring or a spiro ring; G is N, C or CH;
and/or, n is 0 or 1; R⁴ is independently C₁₋₆ alkyl, or C₁₋₆ alkyl substituted with one or more R⁴⁻¹; R⁴⁻¹ is independently cyano;
and/or, R² is —C(═O)—C(R^{a})═C(R^{b})(R^{f}), —C(═O)—C≡C-Me or —S(═O)₂—C(R^{a})═C(R^{b})(R^{f}); R^{a} is independently hydrogen or halogen; R^{b} and R^{f} are independently hydrogen, C₁₋₆ alkyl, or C₁₋₆ alkyl substituted with one or more R^{b-1}; R^{b-1} is independently halogen, hydroxyl, C₁₋₆ alkoxy, or —NR^{10j}R^{10k}; R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

3. The oxygen-containing heterocyclic compound represented by formula I as defined in claim 2, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein, R¹ is C₆₋₂₀ aryl, or C₆₋₂₀ aryl substituted with one or more R¹⁻⁶; R¹⁻⁶ is independently halogen, C₁₋₆ alkyl, or C₁₋₆ alkyl substituted with one or more R¹⁻⁶⁻⁴; R¹⁻⁶⁻¹ is independently halogen;
and/or, R³ is —OR³¹ or —NR³³R³⁴; R³¹, R³³ and R³⁴ are independently C₁₋₆ alkyl substituted with one or more R³¹⁻¹; R³¹⁻¹ is independently “5-7 membered heterocycloalkyl containing 1-3 heteroatoms selected from one or more of 0 and N” substituted with one or more R^{d15}, or —NR^{e1}R^{e2}; R^{e1}, R^{e2} and R^{d15} are independently C₁₋₆ alkyl;
and/or, ring Y is a 4-12 membered heterocyclic ring containing 1-4 N atoms;
the heterocyclic ring is a saturated heterocyclic ring or a partly saturated heterocyclic ring; the heterocyclic ring is a monocyclic ring; G is N;
and/or, n is 0 or 1; R⁴ is independently C₁₋₆ alkyl, or C₁₋₆ alkyl substituted with one or more R⁴⁻¹; R⁴⁻¹ is independently cyano;
and/or, R² is —C(═O)—C(R^{a})═C(R^{b})(R^{f}); R^{a} is independently hydrogen or halogen; R^{b} and R^{f} are independently hydrogen or C₁₋₆ alkyl.

4. The oxygen-containing heterocyclic compound represented by formula I as defined in claim 1, a pharmaceutically acceptable salt thereof, a solvate thereof a solvate of the pharmaceutically acceptable salt thereof, a crystal form thereof, a stereoisomer thereof, a tautomer thereof or an isotopic compound thereof, wherein, R¹ is C₆₋₂₀ aryl, “5-12 membered heteroaryl containing 1-4 heteroatoms selected from one or more of O, S and N”, C₆₋₂₀ aryl substituted with one or more R¹⁻⁶, or “5-12 membered heteroaryl containing 1-4 heteroatoms selected from one or more of O, S and N” substituted with one or more R¹⁻⁷; R¹⁻⁶ and R¹⁻⁷ are independently halogen, C₁₋₆ alkyl, or C₁₋₆ alkyl substituted with one or more R¹⁻⁶⁻¹; R¹⁻⁶⁻¹ is independently halogen;
and/or, R³ is —OR³¹, —SR³² or —NR³³R³⁴; R³¹, R³² and R³⁴ are independently C₁₋₆ alkyl substituted with one or more R³¹⁻¹; R³³ is independently H, C₁₋₆ alkyl, or C₁₋₆ alkyl substituted with one or more R³¹⁻¹; R³¹⁻¹ is independently “5-7 membered heterocycloalkyl containing 1-3 heteroatoms selected from one or more of O and N” substituted with one or more R^{d15}, or —NR^{e1}R^{e2}; R^{d15} is independently C₁₋₆ alkyl or halogen; R^{e1} and R^{e2} are independently C₁₋₆ alkyl;
and/or, n is 0 or 1; R⁴ is independently C₁₋₆ alkyl, or C₁₋₆ alkyl substituted with one or more R⁴⁻¹; R⁴⁻¹ is independently hydroxyl, cyano, or —C(═O)NR^{4e}R^{4f};
We and R^{4f} are independently hydrogen or C₁₋₆ alkyl;
and/or, R² is CN, —C(═O)—C(R^{a})═C(R^{b})(R^{f}), —C(O)—C≡C-Me or —S(═O)₂—C(R^{a})═C(R^{b})(R^{f}); R^{a} is independently hydrogen or halogen; R^{b} and R^{f} are independently hydrogen, C₁₋₆ alkyl, C₁₋₆ alkyl-C(═O)—, or C₁₋₆ alkyl substituted with one or more R^{b-1}; R^{b-1} is independently —NR^{10j}R^{10k}; R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

5. The oxygen-containing heterocyclic compound represented by formula I as defined in claim 1, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein,
R³ is —OR³¹ or —NR³³R³⁴; R³¹, R³³ and R³⁴ are independently C₁₋₆ alkyl substituted with one or more R³¹⁻¹; R³¹⁻¹ is independently “5-7 membered heterocycloalkyl containing 1-3 heteroatoms selected from one or more of O and N” substituted with one or more R^{d15}, or —NR^{e1}R^{e2}; R^{d15} is independently C₁₋₆ alkyl or halogen; R^{e1} and R^{e2} are independently C₁₋₆ alkyl;
and/or, n is 0 or 1; R⁴ is independently C₁₋₆ alkyl, or C₁₋₆ alkyl substituted with one or more R⁴⁻¹; R⁴⁻¹ is independently hydroxyl or cyano;
and/or, R² is —C(═O)—C(R^{a})═C(R^{b})(R^{f}), —C(O)—C≡C-Me or —S(═O)₂—C(R^{a})═C(R^{b})(R^{f}); R^{a} is independently hydrogen or halogen; R^{b} and R^{f} are independently hydrogen, C₁₋₆ alkyl, or C₁₋₆ alkyl substituted with one or more R^{b-1}; R^{b-1} is independently —NR^{10j}R^{10k}; R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of O and N”.

6. The oxygen-containing heterocyclic compound represented by formula I as defined in claim 1, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein, the oxygen-containing heterocyclic compound represented by formula I is defined as solution 1, solution 2, solution 3, solution 4, solution 5, solution 6 or solution 7;
solution 1:
R¹ is C₆₋₂₀ aryl, “5-12 membered heteroaryl containing 1-4 heteroatoms selected from one or more of O, S and N”, C₆₋₂₀ aryl substituted with one or more R¹⁻⁶, or “5-12 membered heteroaryl containing 1-4 heteroatoms selected from one or more of O, S and N” substituted with one or more R¹⁻⁷;
R¹⁻⁶ and R¹⁻⁷ are independently halogen, hydroxyl, C₁₋₆ alkyl, C₁₋₆ alkoxy, C₃₋₁₀ cycloalkyl, C₁₋₆ alkyl substituted with one or more R¹⁻⁶⁻¹, or C₁₋₆ alkoxy substituted with one or more R¹⁻⁶⁻²; R¹⁻⁶⁻¹ and R¹⁻⁶⁻¹ are independently halogen;
m is 0;
R³ is —OR³¹ or —NR³³R³⁴;
R³¹, R³³ and R³⁴ are independently C₁₋₆ alkyl substituted with one or more R³¹⁻¹;
R³¹⁻¹ is independently “5-7 membered heterocycloalkyl containing 1-3 heteroatoms selected from one or more of O and N” substituted with one or more R^{d15} or —NR^{e1}R^{e2};
R^{d15} is independently C₁₋₆ alkyl, C₁₋₆ alkyl substituted with one or more R¹⁻⁸⁻¹, hydroxyl, C₁₋₆ alkoxy, halogen, —NR^{e5}R^{e6} or —C(═O)NR^{e7}R^{e8};
R¹⁻⁸⁻¹ is independently halogen; R^{e5}, R^{e6}, R^{e7} and R^{e8} are independently hydrogen or C₁₋₆ alkyl;
R^{e1} and R^{e2} are independently C₁₋₆ alkyl;
ring Y is a 4-12 membered heterocyclic ring containing 1-4 N atoms; the heterocyclic ring is a saturated heterocyclic ring or a partly saturated heterocyclic ring; the heterocyclic ring is a monocyclic ring or a spiro ring;
G is N, C or CH;
n is 0 or 1;
R⁴ is independently C₁₋₆ alkyl, or C₁₋₆ alkyl substituted with one or more R⁴⁻¹;
R⁴⁻¹ is independently cyano;
R² is —C(═O)—C(R^{a})═C(R^{b})(R^{f}), —C(O)—C≡C-Me or —S(═O)₂—C(R^{a})═C(R^{b})(R^{f});
R^{a} is independently hydrogen or halogen;
R^{b} and R^{f} are independently hydrogen, C₁₋₆ alkyl, or C₁₋₆ alkyl substituted with one or more R^{b-1};
R^{b-1} is independently halogen, hydroxyl, C₁₋₆ alkoxy, or —NR^{10j}R^{10k};
R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of 0 and N”;
solution 2:
R¹ is C₆₋₂₀ aryl, or C₆₋₂₀ aryl substituted with one or more R¹⁻⁶;
R¹⁻⁶ is independently halogen, C₁₋₆ alkyl, or C₁₋₆ alkyl substituted with one or more R¹⁻⁶⁻¹;
R¹⁻⁶⁻¹ is independently halogen;
m is 0;
R³ is —OR³¹ or —NR³³R³⁴;
R³¹, R³³ and R³⁴ are independently C₁₋₆ alkyl substituted with one or more R³¹⁻¹;
R³¹⁻¹ is independently “5-7 membered heterocycloalkyl containing 1-3 heteroatoms selected from one or more of O and N” substituted with one or more R^{d15}, or —NR^{e1}R^{e2};
R^{e1}, R^{e2} and R^{d15} are independently C₁₋₆ alkyl;
ring Y is a 4-12 membered heterocyclic ring containing 1-4 N atoms; the heterocyclic ring is a saturated heterocyclic ring or a partly saturated heterocyclic ring; the heterocyclic ring is a monocyclic ring;
G is N;
n is 0 or 1;
R⁴ is independently C₁₋₆ alkyl, or C₁₋₆ alkyl substituted with one or more R⁴⁻¹;
R⁴⁻¹ is independently cyano;
R² is —C(═O)—C(R^{a})═C(R^{b})(R^{f});
R^{a} is independently hydrogen or halogen;
R^{b} and R^{f} are independently hydrogen or C₁₋₆ alkyl;
embodiment 3:
R¹ is C₆₋₂₀ aryl, “5-12 membered heteroaryl containing 1-4 heteroatoms selected from one or more of O, S and N”, C₆₋₂₀ aryl substituted with one or more R¹⁻⁶, or “5-12 membered heteroaryl containing 1-4 heteroatoms selected from one or more of O, S and N” substituted with one or more R¹⁻⁷;
R¹⁻⁶ and R¹⁻⁷ are independently halogen, hydroxyl, C₁₋₆ alkyl, C₁₋₆ alkoxy, C₃₋₁₀ cycloalkyl, C₁₋₆ alkyl substituted with one or more R¹⁻⁶⁻¹, or C₁₋₆ alkoxy substituted with one or more R¹⁻⁶⁻²; R¹⁻⁶⁻¹ and R¹⁻⁶⁻² are independently halogen;
m is 0;
R³ is —OR³¹ or —NR³³R³⁴;
R³¹, R³³ and R³⁴ are independently C₁₋₆ alkyl substituted with one or more R³¹⁻¹;
R³¹⁻¹ is independently “5-7 membered heterocycloalkyl containing 1-3 heteroatoms selected from one or more of O and N” substituted with one or more R^{d15}, or —NR^{e1}R^{e2};
R^{d15} is independently C₁₋₆ alkyl, C₁₋₆ alkyl substituted with one or more R¹⁻⁸⁻¹, hydroxyl, C₁₋₆ alkoxy, halogen, —NR^{e5}R^{e6} or —C(═O)NR^{e7}R^{e8};
R¹⁻⁸⁻¹ is independently halogen; R^{e5}, R^{e6}, R^{e7} and R^{e8} are independently hydrogen or C₁₋₆ alkyl;
R^{e1} and R^{e2} are independently C₁₋₆ alkyl;
ring Y is a 4-12 membered heterocyclic ring containing 1-4 N atoms; the heterocyclic ring is a saturated heterocyclic ring or a partly saturated heterocyclic ring; the heterocyclic ring is a monocyclic ring or a spiro ring;
G is N, C or CH;
n is 0 or 1;
R⁴ is independently C₁₋₆ alkyl, or C₁₋₆ alkyl substituted with one or more R⁴⁻¹;
R⁴⁻¹ is independently cyano or hydroxyl;
R² is —C(═O)—C(R^{a})═C(R^{b})(R^{f}), —C(═O)—C≡C-Me or —S(═O)₂—C(R^{a})═C(R^{b})(R^{f});
R^{a} is independently hydrogen or halogen;
R^{b} and R^{f} are independently hydrogen, C₁₋₆ alkyl, or C₁₋₆ alkyl substituted with one or more R^{b-1};
R^{b-1} is independently halogen, hydroxyl, C₁₋₆ alkoxy, or —NR^{10j}R^{10k};
R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of 0 and N”;
solution 4:
R¹ is C₆₋₂₀ aryl, or C₆₋₂₀ aryl substituted with one or more R¹⁻⁶;
R¹⁻⁶ is independently halogen, C₁₋₆ alkyl, or C₁₋₆ alkyl substituted with one or more R¹⁻⁶⁻¹;
R¹⁻⁶⁻¹ is independently halogen;
m is 0;
R³ is —OR³¹ or —NR³³R³⁴;
R³¹, R³³ and R³⁴ are independently C₁₋₆ alkyl substituted with one or more R³¹⁻¹;
R³¹⁻¹ is independently “5-7 membered heterocycloalkyl containing 1-3 heteroatoms selected from one or more of O and N” substituted with one or more R^{d15}, or —NR^{e1}R^{e2};
R^{d15} is independently C₁₋₆ alkyl or halogen;
R^{e1} and R^{e2} are independently C₁₋₆ alkyl;
ring Y is a 4-12 membered heterocyclic ring containing 1-4 N atoms; the heterocyclic ring is a saturated heterocyclic ring or a partly saturated heterocyclic ring; the heterocyclic ring is a monocyclic ring;
G is N;
n is 0 or 1;
R⁴ is independently C₁₋₆ alkyl, or C₁₋₆ alkyl substituted with one or more R⁴⁻¹;
R⁴⁻¹ is independently cyano;
R² is —C(═O)—C(R^{a})═C(R^{b})(R^{f});
R^{a} is independently hydrogen or halogen;
R^{b} and R^{f} are independently hydrogen or C₁₋₆ alkyl, or C₁₋₆ alkyl substituted with one or more R^{b-1};
R^{b-1} is independently —NR^{10j}R^{10k};
R^{10j} and R^{10k} are independently hydrogen or C₁₋₆ alkyl, or R^{10j} and R^{10k} taken together with the N atom to which they are attached form “4-10 membered heterocycloalkyl containing 1-3 heteroatoms selected from one or more of 0 and N”;
solution 5:
R¹ is C₆₋₂₀ aryl, “5-12 membered heteroaryl containing 1-4 heteroatoms selected from one or more of O, S and N”, C₆₋₂₀ aryl substituted with one or more R¹⁻⁶, or “5-12 membered heteroaryl containing 1-4 heteroatoms selected from one or more of O, S and N” substituted with one or more R¹⁻⁷;
R¹⁻⁶ and R¹⁻⁷ are independently halogen, hydroxyl, —C(═O)R⁶⁵, —NR⁶³R⁶⁴, —C(═O)OR⁶⁶, —C(═O)NR⁶⁹R⁶¹⁰, C₁₋₆ alkyl, C₁₋₆ alkoxy, C₃₋₁₀ cycloalkyl, “5-7 membered heterocycloalkyl containing 1 or 2 heteroatoms selected from one or more of O and N”, C₆₋₂₀ aryl, “5-7 membered heteroaryl containing 1 or 2 heteroatoms selected from one or more of O and N”, C₁₋₆ alkyl substituted with one or more R¹⁻⁶⁻¹, C₁₋₆ alkoxy substituted with one or more R¹⁻⁶⁻², C₃₋₁₀ cycloalkyl substituted with one or more R¹⁻⁶⁻³, “5-7 membered heterocycloalkyl containing 1 or 2 heteroatoms selected from one or more of O and N” substituted with one or more R¹⁻⁶⁻⁴, C₆₋₂₀ aryl substituted with one or more R¹⁻⁶⁻⁵, or “5-7 membered heteroaryl containing 1 or 2 heteroatoms selected from one or more of O and N” substituted with one or more R¹⁻⁶⁻⁶;
R¹⁻⁶⁻¹, R¹⁻⁶⁻², R¹⁻⁶⁻³, R¹⁻⁶⁻⁴, R¹⁻⁶⁻⁵ and R¹⁻⁶⁻⁶ are independently cyano, halogen, hydroxyl, C₁₋₆ alkoxy, C₁₋₆ alkyl, —C(═O)R⁶⁵⁻², —NR⁶³⁻²R⁶⁴⁻², —C(═O)OR⁶⁶⁻², or —C(═O)NR⁶⁹⁻²R⁶¹⁰⁻²;
R⁶⁵, R⁶⁵⁻², R⁶³, R⁶³⁻², R⁶⁴, R⁶⁴⁻², R⁶⁶, R⁶⁶⁻², R⁶⁹, R⁶⁹², R⁶¹⁰ and R⁶¹⁰⁻² are independently hydrogen or C₁₋₆ alkyl;
m is 0, 1 or 2;
R⁵ is independently C₁₋₆ alkyl;
R³ is —OR³¹, —SR³² or —NR³³R³⁴;
R³¹, R³², R³³ and R³⁴ are independently C₁₋₆ alkyl substituted with one or more R³¹⁻¹;
R³¹⁻¹ is independently C₃₋₁₀ cycloalkyl, “4-10 membered heterocycloalkyl containing 1-3 heteroatoms selected from one or more of O and N”, C₃₋₁₀ cycloalkyl substituted with one or more R^{d16}, “4-10 membered heterocycloalkyl containing 1-3 heteroatoms selected from one or more of O and N” substituted with one or more R^{d15}, —OR^{d}, —SR^{d1}, —NR^{e1}R^{e2}, or —C(═O)NR^{e3}R^{e4};
R^{d15} and R^{d16} are independently C₁₋₆ alkyl, C₁₋₆ alkyl substituted with one or more R¹⁻⁸⁻¹, hydroxyl, C₁₋₆ alkoxy, halogen, —NR^{e5}R^{e6} or —C(═O)NR^{e7}R^{e8};
R^{d}, R^{d1}, R^{e1}, R^{e2}, R^{e3} and R^{e4} are independently hydrogen, C₁₋₆ alkyl, C₃₋₁₀ cycloalkyl, “4-10 membered heterocycloalkyl containing 1-3 heteroatoms selected from one or more of O and N”, or C₁₋₆ alkyl substituted with one or more R¹⁻⁸⁻¹ and R¹⁻³⁻² are independently cyano, halogen, hydroxyl, C₁₋₆ alkoxy, —C(═O)R^{e9}, —NR^{e10}R^{e11}, —C(═O)OR^{e12}, or —C(═O)NR^{e13}R^{e14};
R^{e5}, R^{e6}, R^{e7}, R^{e8}, R^{e9}, R^{e10}, R^{e11}, R^{e12}, R^{e13} and R^{e14} are independently hydrogen or C₁₋₆ alkyl;
ring Y is a 4-12 membered heterocyclic ring containing 1-4 N atoms; the heterocyclic ring is a saturated heterocyclic ring or a partly saturated heterocyclic ring; the heterocyclic ring is a monocyclic ring, a bridged ring or a spiro ring;
G is N, C or CH;
n is 0, 1, 2 or 3;
R⁴ is independently C₁₋₆ alkyl, C₁₋₆ alkyl substituted with one or more R⁴⁻¹, oxo, —C(═O)OR^{4a} or —C(═O)NR^{4b}R^{4c};
R⁴⁻¹ is independently halogen, cyano, hydroxyl, C₁₋₆ alkoxy, —C(═O)OR^{4d} or —C(═O)NR^{4e}R^{4f}; R^{4d}, R^{4e}, R^{4f}, R⁴ⁱ and R^{4j} are independently hydrogen or C₁₋₆ alkyl;
R^{4a}, R^{4b} and R^{4c} are independently hydrogen or C₁₋₆ alkyl;
R² is —C(═O)—C(R^{a})═C(R^{b})(R^{f}), —C(═O)—C≡CR^{f}, —S(═O)₂—C(R^{a})═C(R^{b})(R^{f}) or —S(═O)₂—C≡CR^{f};
R^{a} is independently hydrogen, deuterium, halogen or C₁₋₆ alkyl;
R^{b} and R^{f} are independently hydrogen, deuterium, C₁₋₆ alkyl, or C₁₋₆ alkyl substituted with one or more R^{b-1};
R^{b-1} is independently halogen, hydroxyl, C₁₋₆ alkoxy, or —NR^{10j}R^{10k};
R^{10j} and R^{10k} are independently hydrogen or C₁₋₆ alkyl, or R^{10j} and R^{10k} taken together with the N atom to which they are attached form “4-10 membered heterocycloalkyl containing 1-3 heteroatoms selected from one or more of 0 and N”;
solution 6:
R¹ is C₆₋₂₀ aryl, “5-12 membered heteroaryl containing 1-4 heteroatoms selected from one or more of O, S and N”, C₆₋₂₀ aryl substituted with one or more R¹-6, or “5-12 membered heteroaryl containing 1-4 heteroatoms selected from one or more of O, S and N” substituted with one or more R¹⁻⁷;
R¹⁻⁶ and R¹⁻⁷ are independently halogen, hydroxyl, C₁₋₆ alkyl, C₁₋₆ alkoxy, C₃₋₁₀ cycloalkyl, C₁₋₆ alkyl substituted with one or more R¹⁻⁶⁻¹, or C₁₋₆ alkoxy substituted with one or more R^{1′}; R¹⁻⁶⁻¹ and R¹⁻⁶⁻² are independently halogen;
m is 0;
R³ is —OR³¹, —SR³² or —NR³³R³⁴;
R³¹, R³² and R³⁴ are independently C₁₋₆ alkyl substituted with one or more R³¹⁻¹; R³³ is independently H, C₁₋₆ alkyl, or C₁₋₆ alkyl substituted with one or more R³¹⁻¹ is independently “5-7 membered heterocycloalkyl containing 1-3 heteroatoms selected from one or more of O and N” substituted with one or more R^{d15}, or —NR^{e1}R^{e2};
R^{d15} is independently C₁₋₆ alkyl or halogen;
R^{e1} and R^{e2} are independently C₁₋₆ alkyl;
ring Y is a 4-12 membered heterocyclic ring containing 1-4 N atoms; the heterocyclic ring is a saturated heterocyclic ring or a partly saturated heterocyclic ring; the heterocyclic ring is a monocyclic ring or a spiro ring;
G is N, C or CH;
n is 0 or 1;
R⁴ is independently C₁₋₆ alkyl, or C₁₋₆ alkyl substituted with one or more R⁴⁻¹;
R⁴⁻¹ is independently cyano, hydroxyl or —C(═O)NR^{4e}R^{4f}; R^{4e} and R^{4f} are independently hydrogen or C₁₋₆ alkyl;
R² is CN, —C(═O)—C(R^{a})═C(R^{b})(R^{f}), —C(═O)—C≡C-Me or, —S(═O)₂—C(R^{a})═C(R^{b})(R^{f});
R^{a} is independently hydrogen or halogen;
R^{b} and R^{f} are independently hydrogen, C₁₋₆ alkyl, C₁₋₆ alkyl-C(═O)—, or C₁₋₆ alkyl substituted with one or more R^{b-1};
R^{b-1} is independently halogen, hydroxyl, C₁₋₆ alkoxy, or —NR^{10j}R^{10k};
R^{10j} and R^{10k} are independently hydrogen or C₁₋₆ alkyl, or R^{10j} and R^{10k} taken together with the N atom to which they are attached form “4-6 membered heterocycloalkyl containing 1-3 heteroatoms selected from one or more of 0 and N”;
solution 7:
R¹ is C₆₋₇₀ aryl, “5-12 membered heteroaryl containing 1-4 heteroatoms selected from one or more of O, S and N”, C₆₋₂₀ aryl substituted with one or more R¹⁻⁶, or “5-12 membered heteroaryl containing 1-4 heteroatoms selected from one or more of O, S and N” substituted with one or more R¹⁻⁷;
R¹⁻⁶ is independently halogen, C₁₋₆ alkyl, or C₁₋₆ alkyl substituted with one or more R¹⁻⁶¹;
R¹⁻⁶⁻¹ is independently halogen;
m is 0;
R³ is —OR³¹, —SR³² or —NR³³R³⁴;
R³¹, R³² and R³⁴ are independently C₁₋₆ alkyl substituted with one or more R³¹⁻¹; R³³ is independently H, C₁₋₆ alkyl, or C₁₋₆ alkyl substituted with one or more R³¹⁻¹ is independently “5-7 membered heterocycloalkyl containing 1-3 heteroatoms selected from one or more of O and N” substituted with one or more R^{d15}, or —NR^{e1}R^{e2};
R^{d15} is independently C₁₋₆ alkyl or halogen;
R^{e1} and R^{e2} are independently C₁₋₆ alkyl;
ring Y is a 4-12 membered heterocyclic ring containing 1-4 N atoms; the heterocyclic ring is a saturated heterocyclic ring or a partly saturated heterocyclic ring; the heterocyclic ring is a monocyclic ring;
G is N;
n is 0 or 1;
R⁴ is independently C₁₋₆ alkyl substituted with one or more R⁴⁻¹;
R⁴⁻¹ is independently cyano or —C(═O)NR^{4e}R^{4f}; R^{4e} and R^{4f} are independently hydrogen or C₁₋₆ alkyl;
R² is CN, —C(═O)—C(R^{a})═C(R^{b})(R^{f});
R^{a} is independently hydrogen or halogen;
R^{b} and Ware independently hydrogen, C₁₋₆ alkyl, or C₁₋₆ alkyl-C(═O)—.

7. The oxygen-containing heterocyclic compound represented by formula I as defined in claim 1, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein the oxygen-containing heterocyclic compound represented by formula I has a structure as follows: 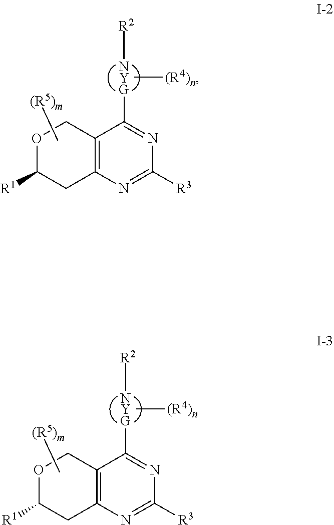
or “a mixture of 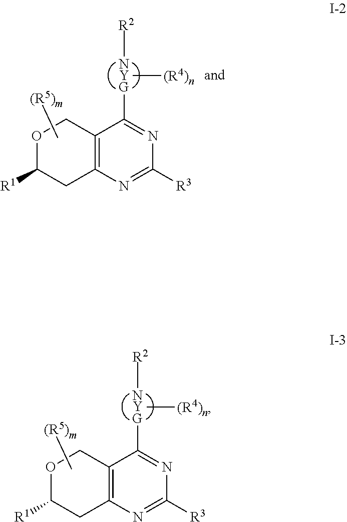
with a molar ratio of, for example, 1:1”;
and/or, when R¹ is C₆₋₂₀ aryl, then the C₆₋₂₀ aryl is phenyl or naphthyl;
and/or, when R¹ is “5-12 membered heteroaryl containing 1-4 heteroatoms selected from one or more of O, S and N”, then the “5-12 membered heteroaryl containing 1-4 heteroatoms selected from one or more of O, S and N” is “9-10 membered heteroaryl containing 1-4 heteroatoms selected from one or more of O, S and N”;
and/or, when R¹ is C₆₋₂₀ aryl substituted with one or more R¹⁻⁶, then the C₆₋₂₀ aryl is phenyl or naphthyl;
and/or, when R¹ is C₆₋₂₀ aryl substituted with one or more R¹⁻⁶, then the more R¹⁻⁶ is two or three R¹⁻⁶;
and/or, when R¹⁻⁶ is independently halogen, then the halogen is fluorine, chlorine, bromine or iodine;
and/or, when R¹⁻⁶ is independently C₁₋₆ alkyl, then the C₁₋₆ alkyl is C₁₋₄ alkyl;
and/or, when R^{1′} is independently C₁₋₆ alkyl substituted with one or more R¹⁻⁶⁻¹, then the C₁₋₆ alkyl is C₁₋₄ alkyl;
and/or, when R¹⁻⁶ is independently C₁₋₆ alkyl substituted with one or more R¹⁻⁶⁻¹, the more R¹⁻⁶⁻¹ is two or three R¹⁻⁶⁻¹;
and/or, when R¹⁻⁶⁻¹ is independently halogen, then the halogen is fluorine, chlorine, bromine or iodine;
and/or, when R³¹, R³³ and R³⁴ are independently C₁₋₆ alkyl substituted with one or more R³¹⁻¹, then the C₁₋₆ alkyl is C₁₋₄ alkyl;
and/or, when R³¹, R³³ and R³⁴ are independently C₁₋₆ alkyl substituted with one or more R³¹⁻¹, then the more R³¹⁻¹ is two or three R³¹⁻¹;
and/or, when R³¹⁻¹ is independently “4-10 membered heterocycloalkyl containing 1-3 heteroatoms selected from one or more of O and N” substituted with one or more R^{d15}, then the “4-10 membered heterocycloalkyl containing 1-3 heteroatoms selected from one or more of O and N” is “5-7 membered heterocycloalkyl containing 1-3 heteroatoms selected from one or more of O and N”;
and/or, when R³¹⁻¹ is independently “4-10 membered heterocycloalkyl containing 1-3 heteroatoms selected from one or more of O and N” substituted with one or more R^{d15}, then the more R^{d15} is two or three R^{d15};
and/or, when R^{d15} is independently C₁₋₆ alkyl, then the C₁₋₆ alkyl is C₁₋₄ alkyl;
and/or, when R^{d15} is independently halogen, then the halogen is fluorine, chlorine, bromine or iodine;
and/or, when R^{e1} and R^{e2} are independently C₁₋₆ alkyl, then the C₁₋₆ alkyl is C₁₋₄ alkyl;
and/or, when ring Y is a 4-12 membered heterocyclic ring containing 1-4 N atoms, then the 4-12 membered heterocyclic ring containing 1-4 N atoms is 6-9 membered heterocyclic ring containing 1-2 N atoms;
and/or, when R⁴ is independently C₁₋₆ alkyl, then the C₁₋₆ alkyl is C₁₋₄ alkyl;
and/or, when R⁴ is independently C₁₋₆ alkyl substituted with one or more R⁴⁻¹, then the C₁₋₆ alkyl is C₁₋₄ alkyl;
and/or, when R⁴ is independently C₁₋₆ alkyl substituted with one or more R⁴⁻¹, then the more R⁴⁻¹ is two or three R⁴⁻¹;
and/or, when R^{a} is independently halogen, then the halogen is fluorine, chlorine, bromine or iodine;
and/or, when R^{b} and R^{f} are independently C₁₋₆ alkyl, then the C₁₋₆ alkyl is C₁₋₄ alkyl;
and/or, when R^{b} and R^{f} are independently C₁₋₆ alkyl substituted with one or more R^{b-1}, then the C₁₋₆ alkyl is C₁₋₄ alkyl;
and/or, when R^{b} and R^{f} are independently C₁₋₆ alkyl substituted with one or more R^{b-1}, then the more R^{b-1} is two or three R^{b-1};
and/or, when R^{10j} and R^{10k} are independently C₁₋₆ alkyl, then the C₁₋₆ alkyl is C₁₋₄ alkyl.

8. The oxygen-containing heterocyclic compound represented by formula I as defined in claim 7, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein, when R¹ is “5-12 membered heteroaryl containing 1-4 heteroatoms selected from one or more of O, S and N”, then the “5-12 membered heteroaryl containing 1-4 heteroatoms selected from one or more of O, S and N” is isoquinolyl;
and/or, when R¹ is C₆₋₂₀ aryl substituted with one or more R¹⁻⁶, then the C₆₋₂₀ aryl is phenyl or 1-naphthyl;
and/or, when R¹⁻⁶ is independently halogen, then the halogen is fluorine or chlorine;
and/or, when R¹⁻⁶ is independently C₁₋₆ alkyl, then the C₁₋₆ alkyl is methyl, ethyl, n-propyl, isopropyl, n-butyl, isobutyl, sec-butyl or tert-butyl;
and/or, when R¹⁻⁶ is independently C₁₋₆ alkyl substituted with one or more R¹⁻⁶⁻¹, then the C₁₋₆ alkyl is methyl, ethyl, n-propyl, isopropyl, n-butyl, isobutyl, sec-butyl or tert-butyl;
and/or, when R¹⁻⁶⁻¹ is independently halogen, then the halogen is fluorine;
and/or, when R³¹, R³³ and R³⁴ are independently C₁₋₆ alkyl substituted with one or more R³¹⁻¹, then the C₁₋₆ alkyl is methyl, ethyl, n-propyl, isopropyl, n-butyl, isobutyl, sec-butyl or tert-butyl;
and/or, when R³¹⁻¹ is independently “4-10 membered heterocycloalkyl containing 1-3 heteroatoms selected from one or more of O and N” substituted with one or more R^{d15}, then the “4-10 membered heterocycloalkyl containing 1-3 heteroatoms selected from one or more of O and N” is “5-7 membered heterocycloalkyl containing 1 heteroatom selected from one of O and N”;
and/or, when R^{d15} is independently C₁₋₆ alkyl, then the C₁₋₆ alkyl is methyl, ethyl, n-propyl, isopropyl, n-butyl, isobutyl, sec-butyl or tert-butyl;
and/or, when R^{d15} is independently halogen, then the halogen is fluorine;
and/or, when R^{e1} and R^{e2} are independently C₁₋₆ alkyl, then the C₁₋₆ alkyl is methyl, ethyl, n-propyl, isopropyl, n-butyl, isobutyl, sec-butyl or tert-butyl;
and/or, when ring Y is a 4-12 membered heterocyclic ring containing 1-4 N atoms, then the 4-12 membered heterocyclic ring containing 1-4 N atoms is 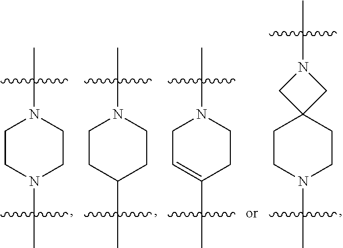
which, at its upper end, is connected to R²;
and/or, when R⁴ is independently C₁₋₆ alkyl, then the C₁₋₆ alkyl is methyl, ethyl, n-propyl, isopropyl, n-butyl, isobutyl, sec-butyl or tert-butyl;
and/or, when R⁴ is independently C₁₋₆ alkyl substituted with one or more R⁴⁻¹, then the C₁₋₆ alkyl is methyl, ethyl, n-propyl, isopropyl, n-butyl, isobutyl, sec-butyl or tert-butyl;
and/or, when R^{a} is independently halogen, then the halogen is fluorine;
and/or, when R^{b} and R^{f} are independently C₁₋₆ alkyl, then the C₁₋₆ alkyl is methyl, ethyl, n-propyl, isopropyl, n-butyl, isobutyl, sec-butyl or tert-butyl;
and/or, when R^{b} and R^{f} are independently C₁₋₆ alkyl substituted with one or more R^{b-1}, then the C₁₋₆ alkyl is methyl, ethyl, n-propyl, isopropyl, n-butyl, isobutyl, sec-butyl or tert-butyl;
and/or, when R^{10j} and R^{10k} are independently C₁₋₆ alkyl, then the C₁₋₆ alkyl is methyl, ethyl, n-propyl, isopropyl, n-butyl, isobutyl, sec-butyl or tert-butyl.

9. The oxygen-containing heterocyclic compound represented by formula I as defined in claim 8, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein, when R¹ is “5-12 membered heteroaryl containing 1-4 heteroatoms selected from one or more of O, S and N”, then the “5-12 membered heteroaryl containing 1-4 heteroatoms selected from one or more of O, S and N” is 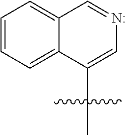
and/or, when R¹⁻⁶ is independently C₁₋₆ alkyl, then the C₁₋₆ alkyl is methyl;
and/or, when R¹⁻⁶ is independently C₁₋₆ alkyl substituted with one or more R¹⁻⁶⁻¹, then the C₁₋₆ alkyl substituted with one or more R¹⁻⁶⁻¹ is trifluoromethyl;
and/or, when R³¹, R³³ and R³⁴ are independently C₁₋₆ alkyl substituted with one or more R³¹⁻¹, then the C₁₋₆ alkyl is methyl, ethyl, n-propyl or isopropyl;
and/or, when R³¹⁻¹ is independently “4-10 membered heterocycloalkyl containing 1-3 heteroatoms selected from one or more of O and N” substituted with one or more R^{d15}, then the “4-10 membered heterocycloalkyl containing 1-3 heteroatoms selected from one or more of O and N” is tetrahydropyrrolyl;
and/or, when R^{d15} is independently C₁₋₆ alkyl, then the C₁₋₆ alkyl is methyl;
and/or, when R^{e1} and R^{e2} are independently C₁₋₆ alkyl, then the C₁₋₆ alkyl is methyl or ethyl;
and/or, the oxygen-containing heterocyclic compound represented by formula I has a structure as follows: 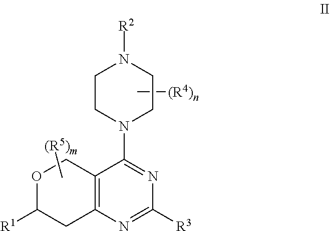
and/or, when R⁴ is independently C₁₋₆ alkyl, then the C₁₋₆ alkyl is methyl;
and/or, when R⁴ is independently C₁₋₆ alkyl substituted with one or more R⁴⁻¹, then the C₁₋₆ alkyl is methyl;
and/or, when R^{b} and R^{f} are independently C₁₋₆ alkyl, then the C₁₋₆ alkyl is methyl;
and/or, when R^{b} and R^{f} are independently C₁₋₆ alkyl substituted with one or more R^{b-1}, then the C₁₋₆ alkyl is methyl;
and/or, when R^{10j} and R^{10k} are independently C₁₋₆ alkyl, then the C₁₋₆ alkyl is methyl.

10. The oxygen-containing heterocyclic compound represented by formula I as defined in claim 9, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein,
the oxygen-containing heterocyclic compound represented by formula I has a structure as follows: 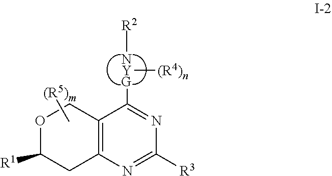
and/or, when R³³ is independently C₁₋₆ alkyl, then the C₁₋₆ alkyl is methyl, ethyl, n-propyl, isopropyl, n-butyl, isobutyl, sec-butyl or tert-butyl, or may be methyl, ethyl, n-propyl or isopropyl;
and/or, when R⁴ is independently C₁₋₆ alkyl substituted with one or more R⁴⁻¹, then the C₁₋₆ alkyl substituted with one or more R⁴¹ is hydroxymethyl, cyanomethyl or 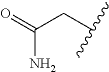 [SMILES: *CC(N)=O]
and/or, when R^{b} and R^{f} are independently C₁₋₆ alkyl-C(═O)—, then the C₁₋₆ alkyl in the C₁₋₆ alkyl-C(═O)— is methyl, ethyl, n-propyl, isopropyl, n-butyl, isobutyl, sec-butyl or tert-butyl, or may be methyl;
and/or, the R² is CN,

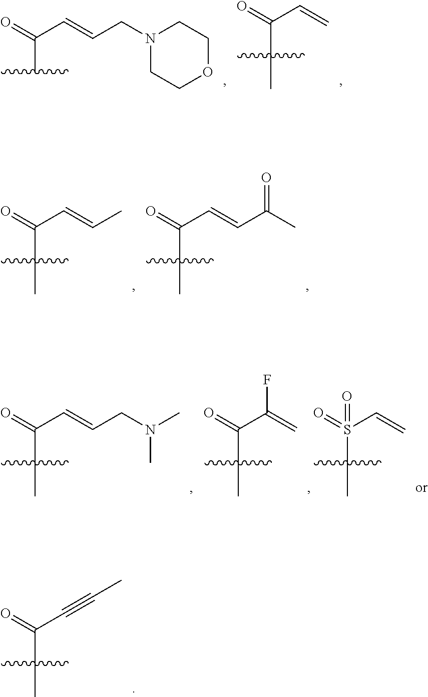

11. The oxygen-containing heterocyclic compound represented by formula I as defined in claim 9, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein,
when R¹ is C₆₋₂₀ aryl substituted with one or more R¹⁻⁶, then the C₆₋₂₀ aryl substituted with one or more R¹⁻⁶ is 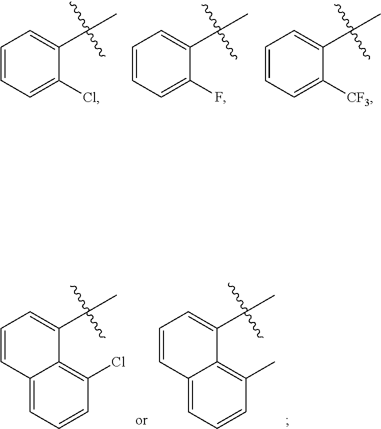
and/or, when R⁴ is independently C₁₋₆ alkyl substituted with one or more R⁴⁻¹, then the C₁₋₆ alkyl substituted with one or more R⁴⁻¹ is hydroxymethyl or cyanomethyl;
and/or, when R^{10j} and R^{10k} taken together with the N atom to which they are attached form “4-10 membered heterocycloalkyl containing 1-3 heteroatoms selected from one or more of O and N”, then the “4-10 membered heterocycloalkyl containing 1-3 heteroatoms selected from one or more of 0 and N” is “5-6 membered heterocycloalkyl containing 2 heteroatoms selected from O and N”, or may be 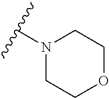 [SMILES: *N1CCOCC1]
and/or, R² is

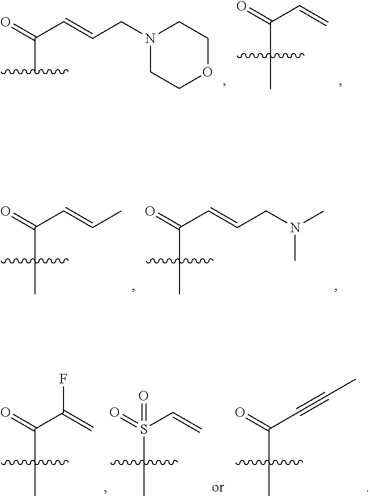

12. The oxygen-containing heterocyclic compound represented by formula I as defined in claim 9, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein, when R¹ is C₆₋₂₀ aryl substituted with one or more R¹⁶, then the C₆₋₂₀ aryl substituted with one or more R¹⁻⁶ is 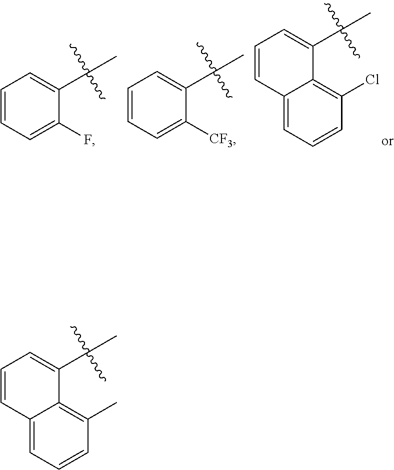
and/or, when R³¹, R³³ and R³⁴ are independently C₁₋₆ alkyl substituted with one or more R³¹⁻¹, then the C₁₋₆ alkyl substituted with one or more R³¹⁻¹ is 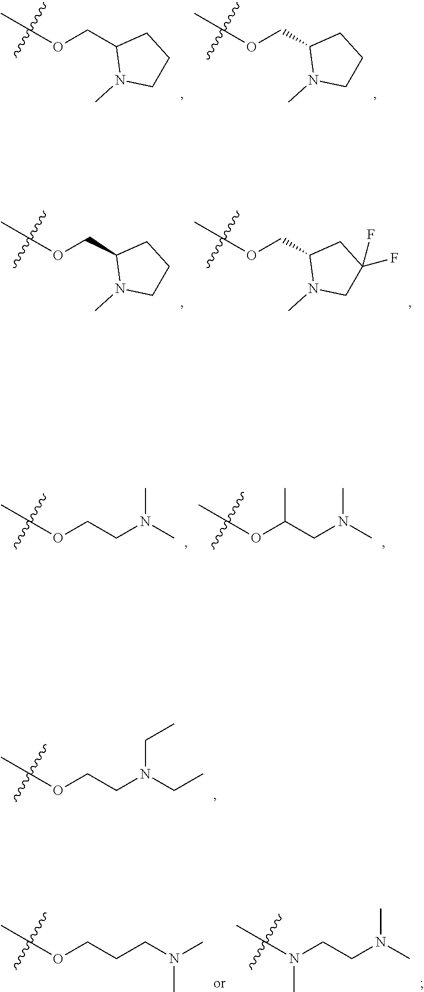
and/or, when R⁴ is independently C₁₋₆ alkyl substituted with one or more R⁴⁻¹, then the C₁₋₆ alkyl substituted with one or more R⁴⁻¹ is cyanomethyl;
and/or, R² is

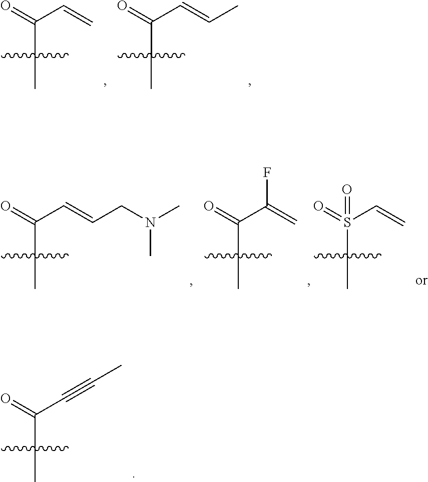

13. The oxygen-containing heterocyclic compound represented by formula I as defined in claim 1, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein, the oxygen-containing heterocyclic compound represented by formula I has any one of the following structures:

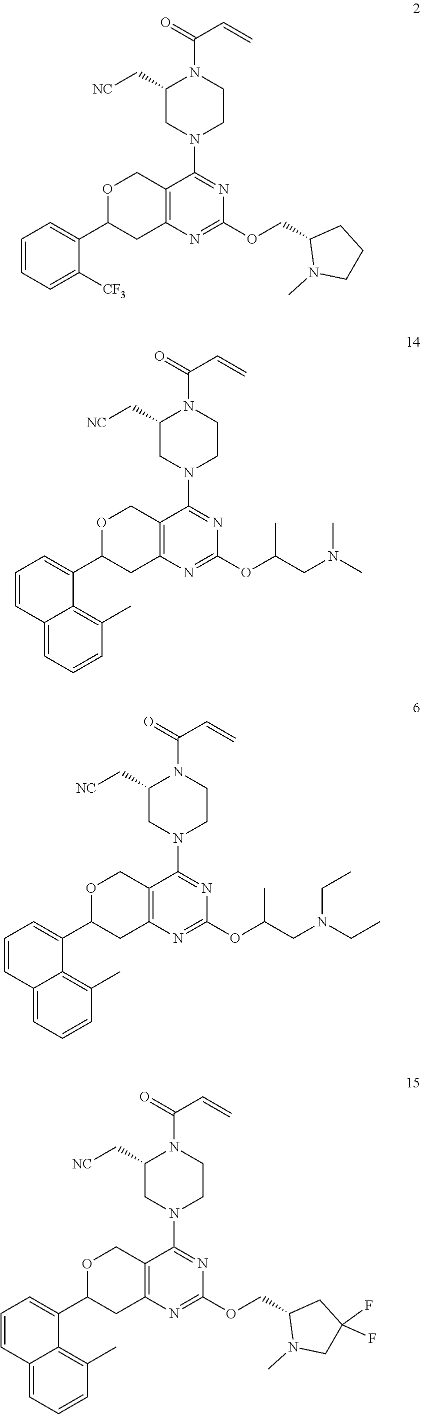 [SMILES: C=CC(=O)N1CCN(c2nc(OC[C@@H]3CCCN3C)nc3c2COC(c2ccccc2C(F)(F)F)C3)C[C@@H]1CC#N ; C=CC(=O)N1CCN(c2nc(OC(C)CN(C)C)nc3c2COC(c2cccc4cccc(C)c24)C3)C[C@@H]1CC#N ; C=CC(=O)N1CCN(c2nc(OC(C)CN(CC)CC)nc3c2COC(c2cccc4cccc(C)c24)C3)C[C@@H]1CC#N ; C=CC(=O)N1CCN(c2nc(OC[C@@H]3CC(F)(F)CN3C)nc3c2COC(c2cccc4cccc(C)c24)C3)C[C@@H]1CC#N]

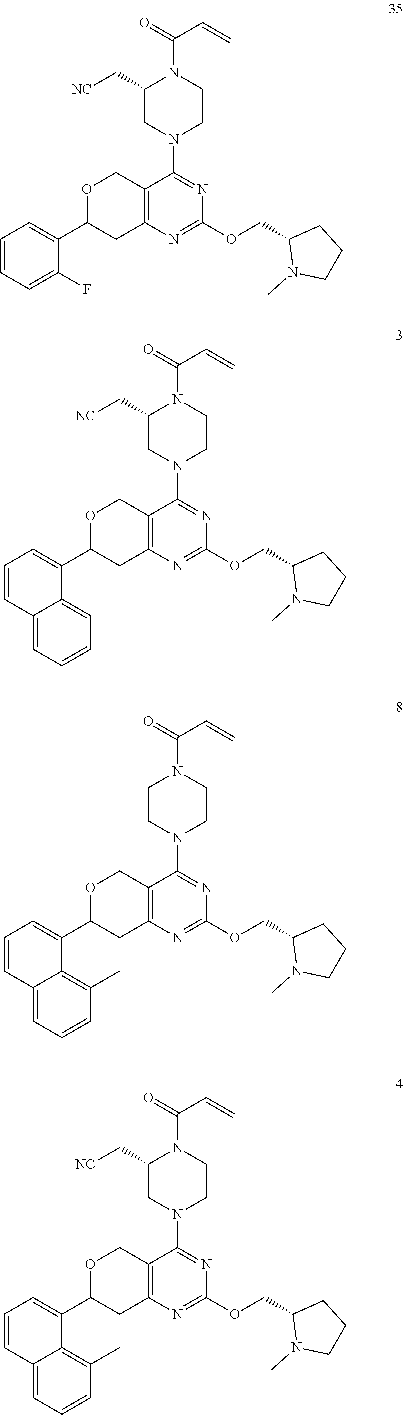 [SMILES: C=CC(=O)N1CCN(c2nc(OC[C@@H]3CCCN3C)nc3c2COC(c2ccccc2F)C3)C[C@@H]1CC#N ; C=CC(=O)N1CCN(c2nc(OC[C@@H]3CCCN3C)nc3c2COC(c2cccc4ccccc24)C3)C[C@@H]1CC#N ; C=CC(=O)N1CCN(c2nc(OC[C@@H]3CCCN3C)nc3c2COC(c2cccc4cccc(C)c24)C3)CC1 ; C=CC(=O)N1CCN(c2nc(OC[C@@H]3CCCN3C)nc3c2COC(c2cccc4cccc(C)c24)C3)C[C@@H]1CC#N]

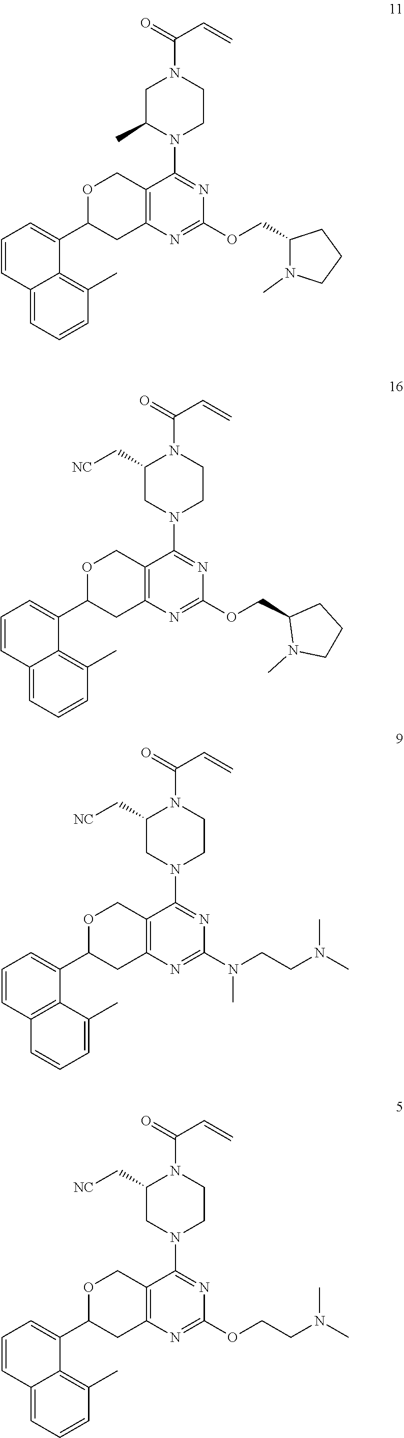 [SMILES: C=CC(=O)N1CCN(c2nc(OC[C@@H]3CCCN3C)nc3c2COC(c2cccc4cccc(C)c24)C3)[C@@H](C)C1 ; C=CC(=O)N1CCN(c2nc(OC[C@H]3CCCN3C)nc3c2COC(c2cccc4cccc(C)c24)C3)C[C@@H]1CC#N ; C=CC(=O)N1CCN(c2nc(N(C)CCN(C)C)nc3c2COC(c2cccc4cccc(C)c24)C3)C[C@@H]1CC#N ; C=CC(=O)N1CCN(c2nc(OCCN(C)C)nc3c2COC(c2cccc4cccc(C)c24)C3)C[C@@H]1CC#N]

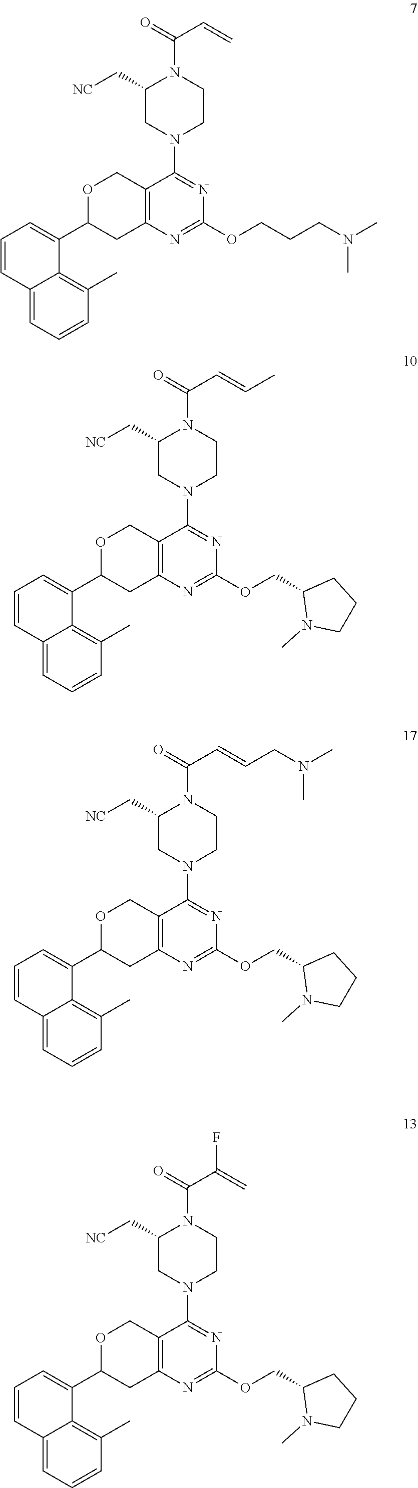 [SMILES: C=CC(=O)N1CCN(c2nc(OCCCN(C)C)nc3c2COC(c2cccc4cccc(C)c24)C3)C[C@@H]1CC#N ; C/C=C/C(=O)N1CCN(c2nc(OC[C@@H]3CCCN3C)nc3c2COC(c2cccc4cccc(C)c24)C3)C[C@@H]1CC#N ; Cc1cccc2cccc(C3Cc4nc(OC[C@@H]5CCCN5C)nc(N5CCN(C(=O)/C=C/CN(C)C)[C@@H](CC#N)C5)c4CO3)c12 ; C=C(F)C(=O)N1CCN(c2nc(OC[C@@H]3CCCN3C)nc3c2COC(c2cccc4cccc(C)c24)C3)C[C@@H]1CC#N]

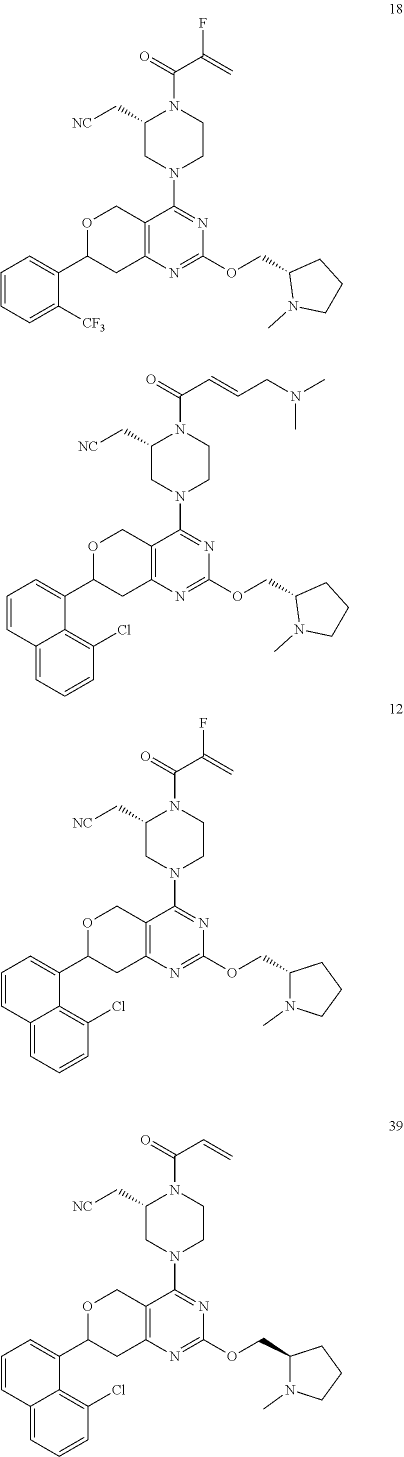 [SMILES: C=C(F)C(=O)N1CCN(c2nc(OC[C@@H]3CCCN3C)nc3c2COC(c2ccccc2C(F)(F)F)C3)C[C@@H]1CC#N ; CN(C)C/C=C/C(=O)N1CCN(c2nc(OC[C@@H]3CCCN3C)nc3c2COC(c2cccc4cccc(Cl)c24)C3)C[C@@H]1CC#N ; C=C(F)C(=O)N1CCN(c2nc(OC[C@@H]3CCCN3C)nc3c2COC(c2cccc4cccc(Cl)c24)C3)C[C@@H]1CC#N ; C=CC(=O)N1CCN(c2nc(OC[C@H]3CCCN3C)nc3c2COC(c2cccc4cccc(Cl)c24)C3)C[C@@H]1CC#N]

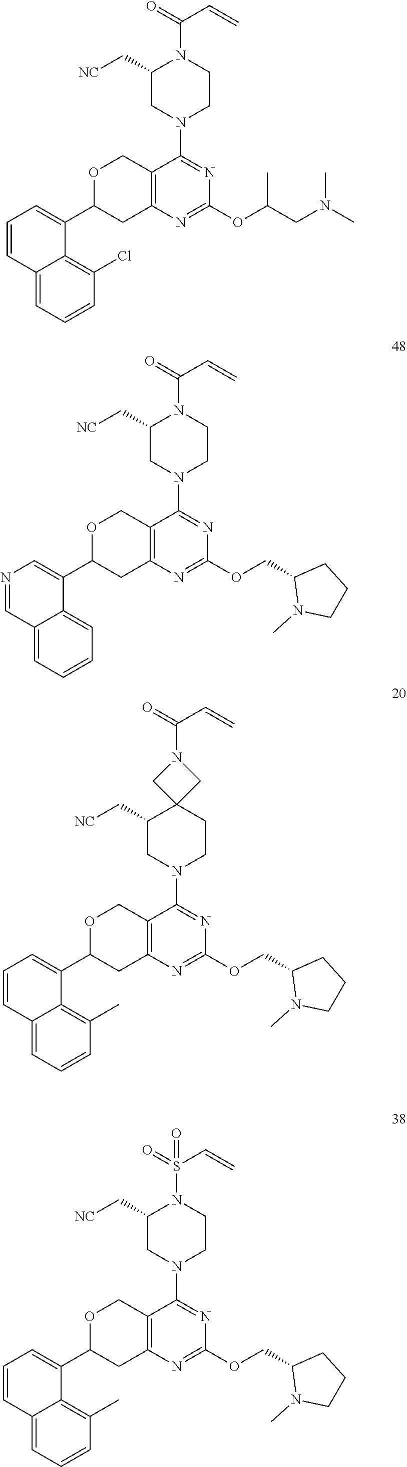 [SMILES: C=CC(=O)N1CCN(c2nc(OC(C)CN(C)C)nc3c2COC(c2cccc4cccc(Cl)c24)C3)C[C@@H]1CC#N ; C=CC(=O)N1CCN(c2nc(OC[C@@H]3CCCN3C)nc3c2COC(c2cncc4ccccc24)C3)C[C@@H]1CC#N ; C=CC(=O)N1CC2(CCN(c3nc(OC[C@@H]4CCCN4C)nc4c3COC(c3cccc5cccc(C)c35)C4)C[C@@H]2CC#N)C1 ; C=CS(=O)(=O)N1CCN(c2nc(OC[C@@H]3CCCN3C)nc3c2COC(c2cccc4cccc(C)c24)C3)C[C@@H]1CC#N]

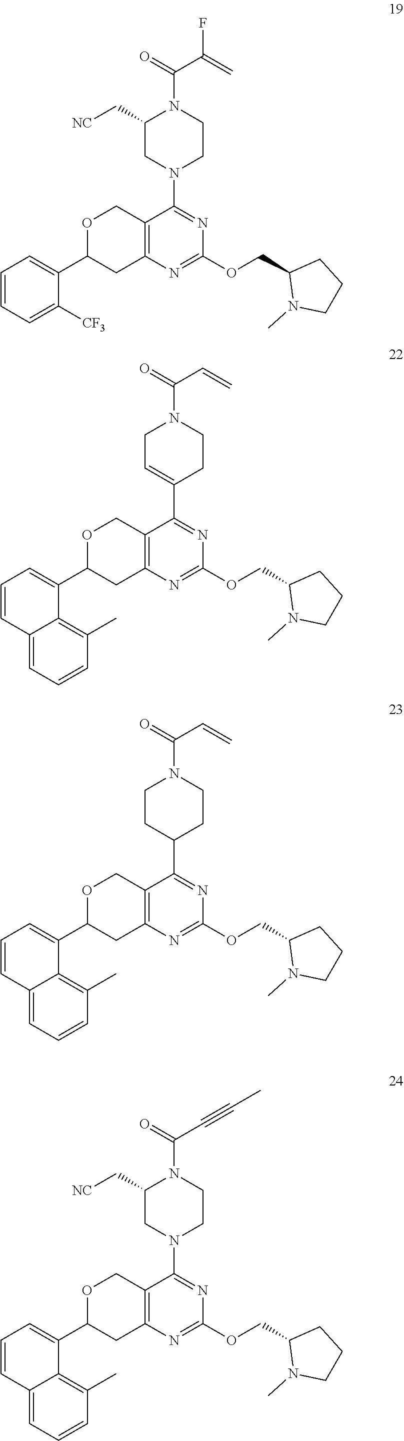 [SMILES: C=C(F)C(=O)N1CCN(c2nc(OC[C@H]3CCCN3C)nc3c2COC(c2ccccc2C(F)(F)F)C3)C[C@@H]1CC#N ; C=CC(=O)N1CC=C(c2nc(OC[C@@H]3CCCN3C)nc3c2COC(c2cccc4cccc(C)c24)C3)CC1 ; C=CC(=O)N1CCC(c2nc(OC[C@@H]3CCCN3C)nc3c2COC(c2cccc4cccc(C)c24)C3)CC1 ; CC#CC(=O)N1CCN(c2nc(OC[C@@H]3CCCN3C)nc3c2COC(c2cccc4cccc(C)c24)C3)C[C@@H]1CC#N]

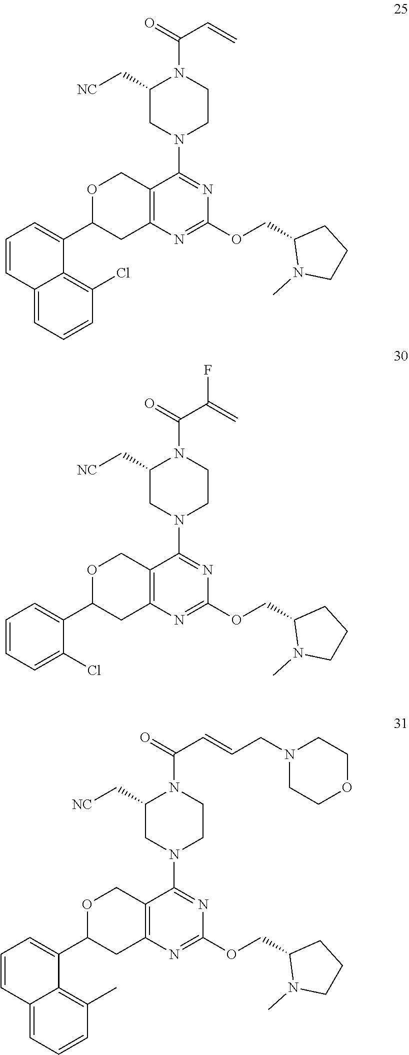 [SMILES: C=CC(=O)N1CCN(c2nc(OC[C@@H]3CCCN3C)nc3c2COC(c2cccc4cccc(Cl)c24)C3)C[C@@H]1CC#N ; C=C(F)C(=O)N1CCN(c2nc(OC[C@@H]3CCCN3C)nc3c2COC(c2ccccc2Cl)C3)C[C@@H]1CC#N ; Cc1cccc2cccc(C3Cc4nc(OC[C@@H]5CCCN5C)nc(N5CCN(C(=O)/C=C/CN6CCOCC6)[C@@H](CC#N)C5)c4CO3)c12]

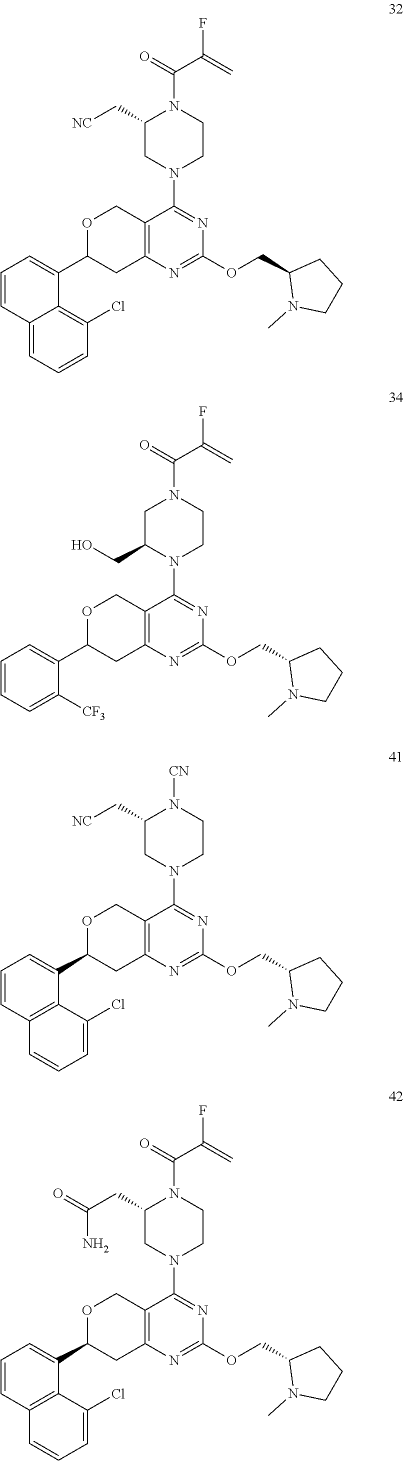 [SMILES: C=C(F)C(=O)N1CCN(c2nc(OC[C@H]3CCCN3C)nc3c2COC(c2cccc4cccc(Cl)c24)C3)C[C@@H]1CC#N ; C=C(F)C(=O)N1CCN(c2nc(OC[C@@H]3CCCN3C)nc3c2COC(c2ccccc2C(F)(F)F)C3)[C@@H](CO)C1 ; CN1CCC[C@H]1COc1nc2c(c(N3CCN(C#N)[C@@H](CC#N)C3)n1)CO[C@H](c1cccc3cccc(Cl)c13)C2 ; C=C(F)C(=O)N1CCN(c2nc(OC[C@@H]3CCCN3C)nc3c2CO[C@H](c2cccc4cccc(Cl)c24)C3)C[C@@H]1CC(N)=O]

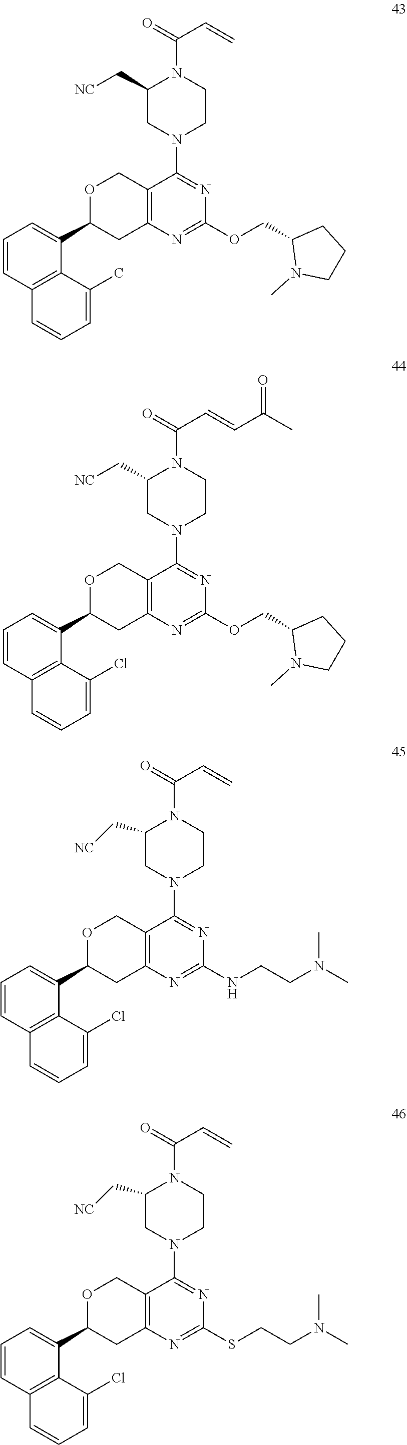 [SMILES: C=CC(=O)N1CCN(c2nc(OC[C@@H]3CCCN3C)nc3c2CO[C@H](c2cccc4cccc(C)c24)C3)C[C@H]1CC#N ; CC(=O)/C=C/C(=O)N1CCN(c2nc(OC[C@@H]3CCCN3C)nc3c2CO[C@H](c2cccc4cccc(Cl)c24)C3)C[C@@H]1CC#N ; C=CC(=O)N1CCN(c2nc(NCCN(C)C)nc3c2CO[C@H](c2cccc4cccc(Cl)c24)C3)C[C@@H]1CC#N ; C=CC(=O)N1CCN(c2nc(SCCN(C)C)nc3c2CO[C@H](c2cccc4cccc(Cl)c24)C3)C[C@@H]1CC#N]

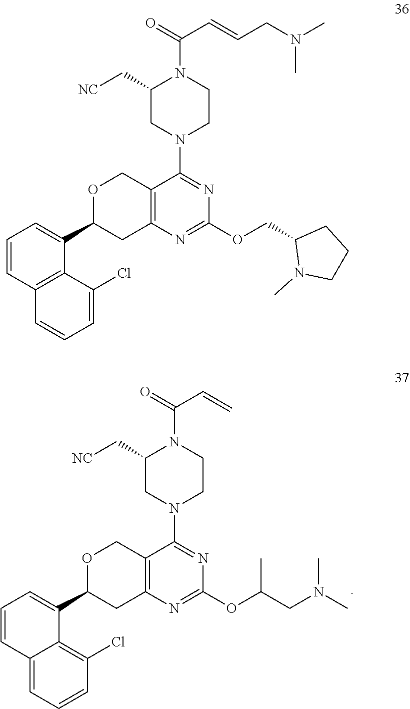 [SMILES: CN(C)C/C=C/C(=O)N1CCN(c2nc(OC[C@@H]3CCCN3C)nc3c2CO[C@H](c2cccc4cccc(Cl)c24)C3)C[C@@H]1CC#N ; C=CC(=O)N1CCN(c2nc(OC(C)CN(C)C)nc3c2CO[C@H](c2cccc4cccc(Cl)c24)C3)C[C@@H]1CC#N]

14. The oxygen-containing heterocyclic compound represented by formula I as defined in claim 1, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein, the oxygen-containing heterocyclic compound represented by formula I is any one of the following compounds:
compound 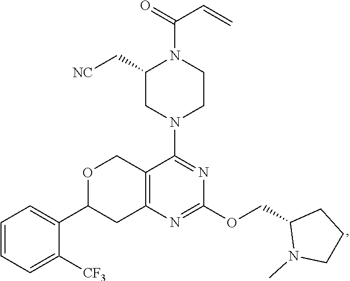 [SMILES: C=CC(=O)N1CCN(c2nc(OC[C@@H]3CCCN3C)nc3c2COC(c2ccccc2C(F)(F)F)C3)C[C@@H]1CC#N]
which has a retention time of 0.92 min wider the following conditions: equipment: SFC Method Station (Thar, Waters); chromatographic column: OD-H 4.6*100 mm, 5 μm (Daicel): column temperature: 40° C.: mobile phase: CO₂/MeOH (0.1% TEA)=65/35: flow rate: 4.0 ml/min: wavelength: 254 nm; back pressure: 120 bar:
compound 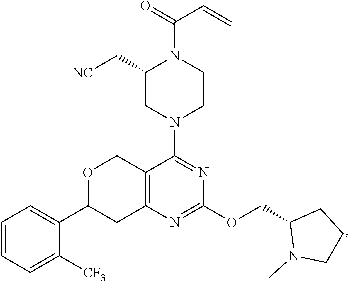 [SMILES: C=CC(=O)N1CCN(c2nc(OC[C@@H]3CCCN3C)nc3c2COC(c2ccccc2C(F)(F)F)C3)C[C@@H]1CC#N]
which has a retention time of 2.74 min under the following conditions: equipment: SFC Method Station (Thar, Waters); chromatographic column: OD-H 4.6*100 mm, 5 μm (Daicel): column temperature: 40° C.; mobile phase: CO₂/MeOH (0.1% TEA)=65/35: flow rate: 4.0 ml/min: wavelength: 254 nm: back pressure: 120 bar:
compound 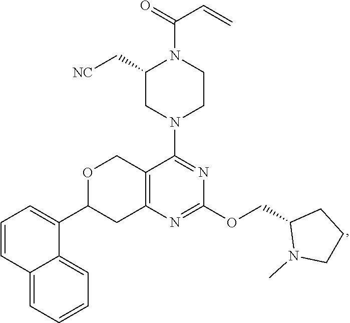 [SMILES: C=CC(=O)N1CCN(c2nc(OC[C@@H]3CCCN3C)nc3c2COC(c2cccc4ccccc24)C3)C[C@@H]1CC#N]
which has a retention time of 0.97 min under the following conditions: equipment: SFC Method Station (Thar, Waters); chromatographic column: AD-H 4.6*100 mm, 5 μm (Daicel): column temperature: 40° C.; mobile phase: CO₂/ETOH (0.5% TEA)=55/45: flow rate: 4.0 ml/min: wavelength: 254 nm: back pressure: 120 bar:
compound 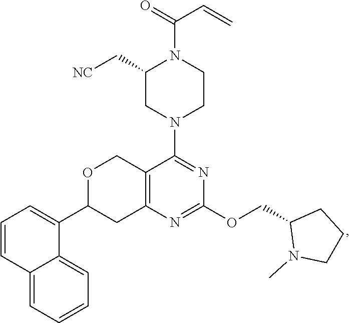 [SMILES: C=CC(=O)N1CCN(c2nc(OC[C@@H]3CCCN3C)nc3c2COC(c2cccc4ccccc24)C3)C[C@@H]1CC#N]
which has a retention time of 2.40 min under the following conditions: equipment: SFC Method Station (Thar, Waters); chromatographic column: AD-H 4.6*100 mm, 5 μm (Daicel): column temperature: 40° C.: mobile phase: CO₂/ETOH (0.5% TEA)=55/45; flow rate: 4.0 ml/min: wavelength: 254 nm; back pressure: 120 bar:
compound 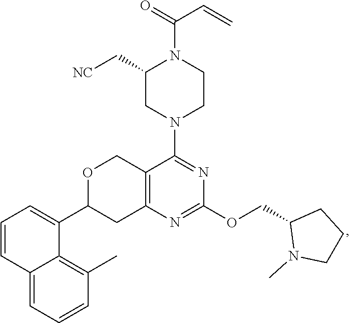 [SMILES: C=CC(=O)N1CCN(c2nc(OC[C@@H]3CCCN3C)nc3c2COC(c2cccc4cccc(C)c24)C3)C[C@@H]1CC#N]
which has a retention time of 0.97 min under the following conditions: equipment: SFC Method Station (Thar, Waters); chromatographic column: OJ-H 4.6*100 mm, 5 μm (Daicel): column temperature: 40° C.: mobile phase: CO₂/Methanol (0.1% TEA)=60/40; flow rate: 4.0 ml/min: wavelength: 254 nm; back pressure: 120 bar:
compound 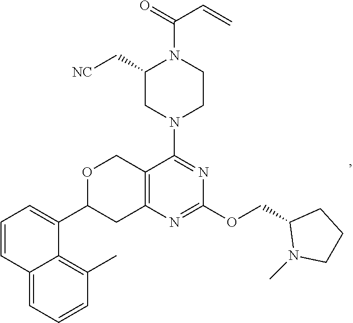 [SMILES: C=CC(=O)N1CCN(c2nc(OC[C@@H]3CCCN3C)nc3c2COC(c2cccc4cccc(C)c24)C3)C[C@@H]1CC#N]
which has a retention time of 1.94 min under the following conditions: equipment: SFC Method Station (Thar, Waters); chromatographic column: OJ-H 4.6*100 min, 5 μm (Daicel); column temperature: 40° C.: mobile phase: CO₂/Methanol (0.1% TEA)=60/40: flow rate: 4.00 ml/min: wavelength: 254 nm; back pressure: 120 bar:
compound 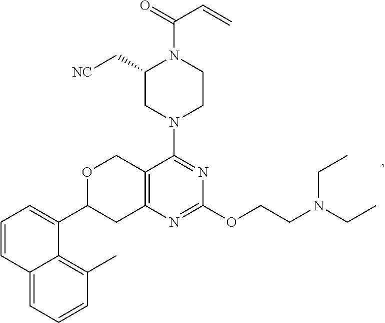 [SMILES: C=CC(=O)N1CCN(c2nc(OCCN(CC)CC)nc3c2COC(c2cccc4cccc(C)c24)C3)C[C@@H]1CC#N]
which has a retention time of 1.22 min under the following conditions: equipment: SFC Method Station (Thar, Waters); chromatographic column: CHIRALCEL OJ-H 4.6*100 mm, 5 μm (Daicel); column temperature: 40° C.; mobile phase: CO₂/MeOH (0.1% TEA)=65/35: flow rate: 1.0 ml/min: wavelength: 214 nm: back pressure: 120 bar:
compound 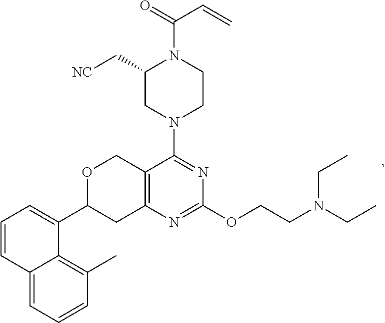 [SMILES: C=CC(=O)N1CCN(c2nc(OCCN(CC)CC)nc3c2COC(c2cccc4cccc(C)c24)C3)C[C@@H]1CC#N]
which has a retention time of 2.67 min under the following conditions: equipment: SFC Method Station (Thar, Waters); chromatographic column: CHIRALCEL OJ-H 4.6*100 mm, 5 μm (Daicel); column temperature: 40° C.; mobile phase: CO₂/MeOH (0.1% TEA)=65/35: flow rate: 1.0 ml/min: wavelength: 214 nm; back pressure: 120 bar:
compound 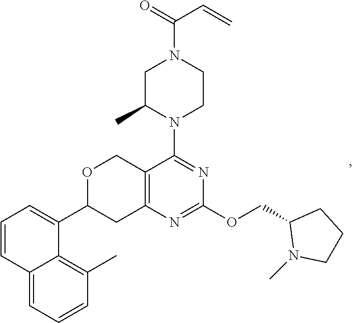 [SMILES: C=CC(=O)N1CCN(c2nc(OC[C@@H]3CCCN3C)nc3c2COC(c2cccc4cccc(C)c24)C3)[C@@H](C)C1]
which has a retention time of 3.26 min under the following conditions: instrument: SFC Method Station (Thar, Waters); chromatographic column: R,R-WHELK-O1 4.6*100 mm, 5 μm (REGIS); column temperature: 40° C.; mobile phase: CO₂/(MeOH/ACN=3:2 (0.1% TEA))=55/45: flow rate: 4.0 ml/min: wavelength: 254 nm: back pressure: 120 bar:
compound 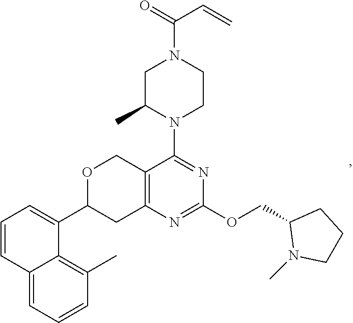 [SMILES: C=CC(=O)N1CCN(c2nc(OC[C@@H]3CCCN3C)nc3c2COC(c2cccc4cccc(C)c24)C3)[C@@H](C)C1]
which has a retention time of 4.16 min under the following conditions: instrument: SFC Method Station (Thar, Waters); chromatographic column: R,R-WHELK-O1 4.6*100 mm, 5 μm (REGIS); column temperature: 40° C.: mobile phase: CO₂/(MeOH/CAN=3:2 (0.1% TEA))=55/45: flow rate: 4.0 ml/min: wavelength: 254 nm; back pressure: 120 bar:
compound 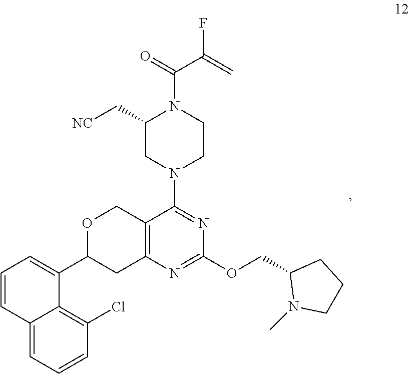 [SMILES: C=C(F)C(=O)N1CCN(c2nc(OC[C@@H]3CCCN3C)nc3c2COC(c2cccc4cccc(Cl)c24)C3)C[C@@H]1CC#N]
which has a retention time of 1.36 min under the following conditions: instrument: SFC Method Station (Thar, Waters); chromatographic column: OJ-H 4.6*100 mm, 5 μm (Daicel): column temperature: 40° C.: mobile phase: CO₂/MeOH (0.1% TEA)=60/40: flow rate: 4.0 ml/min: wavelength: 254 nm: back pressure: 120 bar:
compound 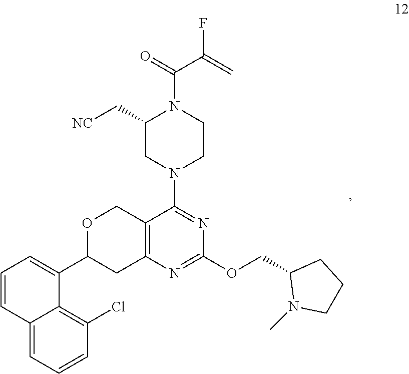 [SMILES: C=C(F)C(=O)N1CCN(c2nc(OC[C@@H]3CCCN3C)nc3c2COC(c2cccc4cccc(Cl)c24)C3)C[C@@H]1CC#N]
which has a retention time of 2.77 min under the following conditions: instrument: SFC Method Station (Thar, Waters); chromatographic column: OJ-H 4.6*100 mm, 5 μm (Daicel): column temperature: 40° C.: mobile phase: CO₂/MeOH (0.1% TEA)=60/40: flow rate: 4.0 nil/min: wavelength: 254 nm: back pressure: 120 bar:
compound 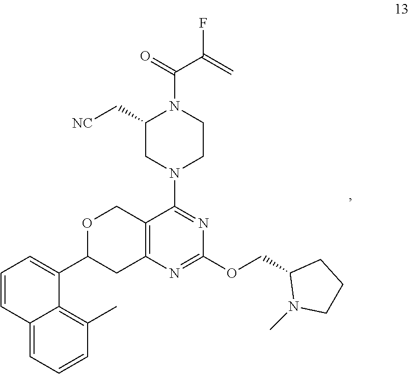 [SMILES: C=C(F)C(=O)N1CCN(c2nc(OC[C@@H]3CCCN3C)nc3c2COC(c2cccc4cccc(C)c24)C3)C[C@@H]1CC#N]
which has a retention time of 1.17 min under the following conditions: instrument: SFC Method Station (Thar, Waters); chromatographic column: OJ-H 4.6*100 rum, 5 μm (Daicel): column temperature: 40° C.: mobile phase: CO₂/MeOH (0.1% TEA)=60/40: flow rate: 4.0 ml/min; wavelength: 254 nm: back pressure: 120 bar:
compound 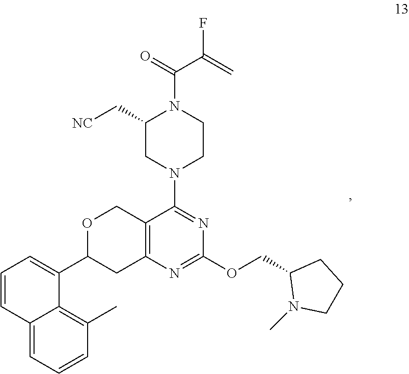 [SMILES: C=C(F)C(=O)N1CCN(c2nc(OC[C@@H]3CCCN3C)nc3c2COC(c2cccc4cccc(C)c24)C3)C[C@@H]1CC#N]
which has a retention time of 2.76 min under the following conditions: instrument: SFC Method Station (Thar, Waters); chromatographic column: OJ-H 4.6*100 mm, 5 μm (Daicel): column temperature: 40° C.: mobile phase: CO₂/MeOH (0.1% TEA)=60/40: flow rate: 4.0 wavelength: 254 nm: back pressure: 120 bar:
compound 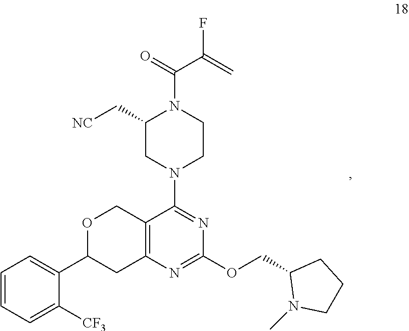 [SMILES: C=C(F)C(=O)N1CCN(c2nc(OC[C@@H]3CCCN3C)nc3c2COC(c2ccccc2C(F)(F)F)C3)C[C@@H]1CC#N]
which has a retention time of 0.78 min under the following conditions: instrument: SFC Method Station (Thar, Waters); chromatographic column: AD-H 4.6*100 mm, 5 μm (Daicel): column temperature: 40° C.: mobile phase: CO₂/MeOH (0.1% TEA)=65/35: flow rate: 4.0 ml/min: wavelength: 254 nm: back pressure: 120 bar:
compound 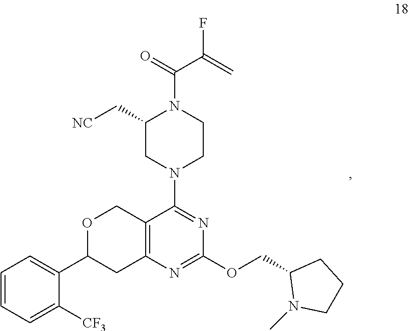 [SMILES: C=C(F)C(=O)N1CCN(c2nc(OC[C@@H]3CCCN3C)nc3c2COC(c2ccccc2C(F)(F)F)C3)C[C@@H]1CC#N]
which has a retention time of 2.42 min under the following conditions: instrument: SFC Method Station (Thar, Waters); chromatographic column: AD-H 4.6*100 mm, 5 μm (Daicel): column temperature: 40° C.: mobile phase: CO₂/MeOH (0.1% TEA)=65/35: flow rate: 4.0 ml/min: wavelength: 254 nm: back pressure: 120 bar:
compound 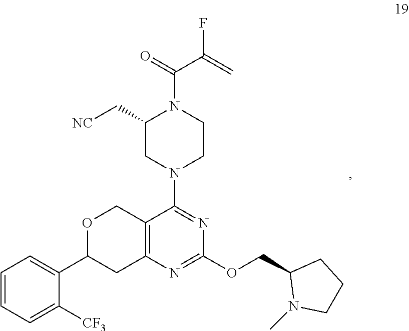 [SMILES: C=C(F)C(=O)N1CCN(c2nc(OC[C@H]3CCCN3C)nc3c2COC(c2ccccc2C(F)(F)F)C3)C[C@@H]1CC#N]
which has a retention time of 0.79 min under the following conditions: instrument: SFC Method Station (Thar, Waters); chromatographic column: OD-H 4.6*100 mm, 5 μm (Daicel): column temperature: 40° C.; mobile phase: CO₂/MeOH (0.1% TEA)=65/35: flow rate: 4.0 ml/min: wavelength: 254 inn: back pressure: 120 bar:
compound 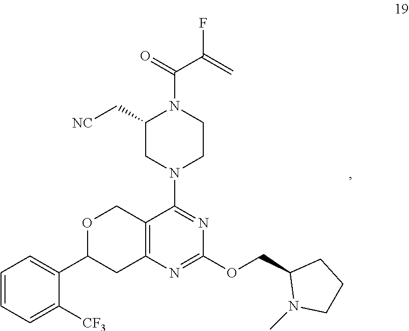 [SMILES: C=C(F)C(=O)N1CCN(c2nc(OC[C@H]3CCCN3C)nc3c2COC(c2ccccc2C(F)(F)F)C3)C[C@@H]1CC#N]
which has a retention time of 2.29 mm under the following conditions: instrument: SFC Method Station (Thar, Waters); chromatographic column: OD-H 4.6*100 mm, 5 μm (Daicel): column temperature: 40° C.; mobile phase: CO₂/MeOH (0.1% TEA)=65/35: flow rate: 4.0 ml/min: wavelength: 254 nm: back pressure: 120 bar:
compound 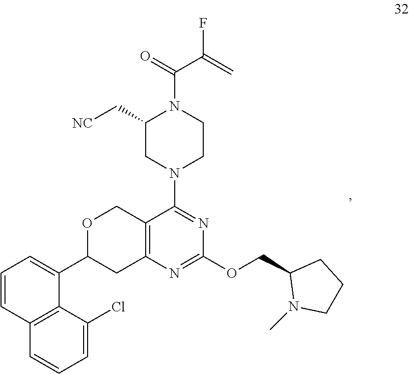 [SMILES: C=C(F)C(=O)N1CCN(c2nc(OC[C@H]3CCCN3C)nc3c2COC(c2cccc4cccc(Cl)c24)C3)C[C@@H]1CC#N]
which has a retention time of 1.45 min under the following conditions: instrument: SFC Method Station (Thar, Waters); chromatographic column: OJ-H 4.6*100 mm, 5 μm (Daicel): column temperature: 40° C.: mobile phase: CO₂/MeOH (0.1% TEA)=60/40: flow rate: 4.0 ml/min: wavelength: 254 mm: back pressure: 120 bar;
compound 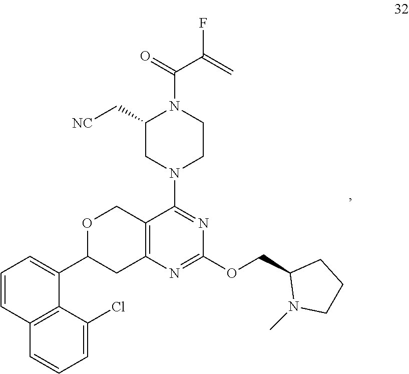 [SMILES: C=C(F)C(=O)N1CCN(c2nc(OC[C@H]3CCCN3C)nc3c2COC(c2cccc4cccc(Cl)c24)C3)C[C@@H]1CC#N]
which has a retention time of 2.81 min under the following conditions: instrument: SFC Method Station (Thar, Waters); chromatographic column: OJ-H 4.6*100 mm, 5 μm (Daicel): column temperature: 40° C.: mobile phase: CO₂/MeOH (0.1% TEA)=60/40: flow rate: 4.0 ml/min: wavelength: 254 nm: back pressure: 120 bar.

15. The oxygen-containing heterocyclic compound represented by formula I as defined in claim 1, a pharmaceutically acceptable salt thereof, a solvate thereof, a solvate of the pharmaceutically acceptable salt thereof, a crystal form thereof, a stereoisomer thereof, a tautomer thereof or an isotopic compound thereof, wherein the oxygen-containing heterocyclic compound represented by formula I is any one of the following compounds:

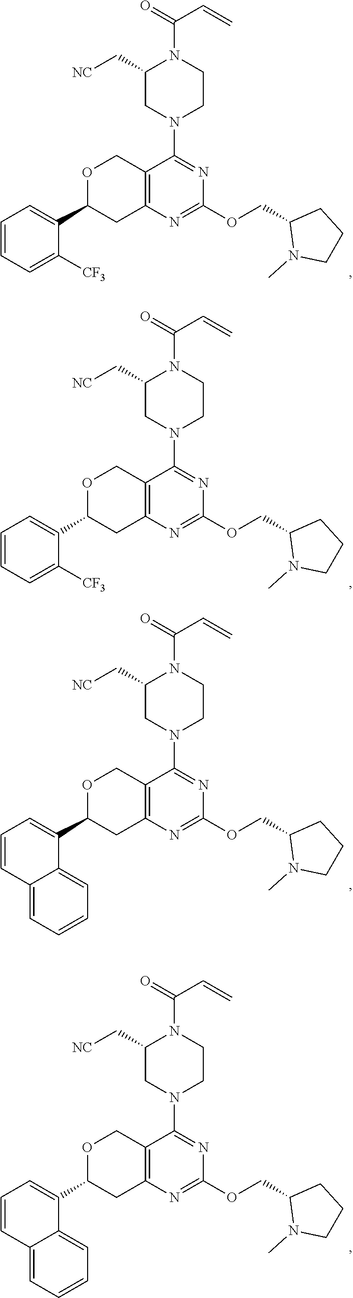 [SMILES: C=CC(=O)N1CCN(c2nc(OC[C@@H]3CCCN3C)nc3c2CO[C@H](c2ccccc2C(F)(F)F)C3)C[C@@H]1CC#N ; C=CC(=O)N1CCN(c2nc(OC[C@@H]3CCCN3C)nc3c2CO[C@@H](c2ccccc2C(F)(F)F)C3)C[C@@H]1CC#N ; C=CC(=O)N1CCN(c2nc(OC[C@@H]3CCCN3C)nc3c2CO[C@H](c2cccc4ccccc24)C3)C[C@@H]1CC#N ; C=CC(=O)N1CCN(c2nc(OC[C@@H]3CCCN3C)nc3c2CO[C@@H](c2cccc4ccccc24)C3)C[C@@H]1CC#N]

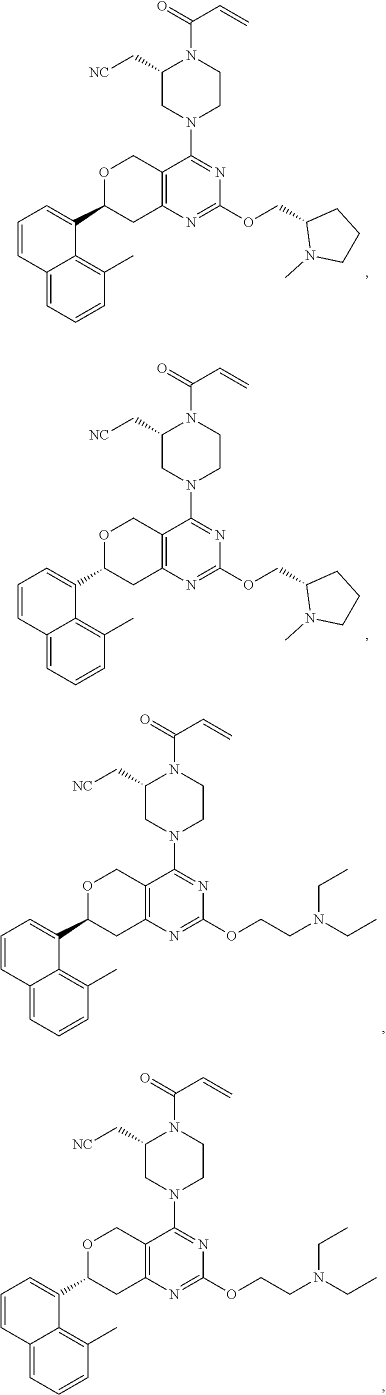 [SMILES: C=CC(=O)N1CCN(c2nc(OC[C@@H]3CCCN3C)nc3c2CO[C@H](c2cccc4cccc(C)c24)C3)C[C@@H]1CC#N ; C=CC(=O)N1CCN(c2nc(OC[C@@H]3CCCN3C)nc3c2CO[C@@H](c2cccc4cccc(C)c24)C3)C[C@@H]1CC#N ; C=CC(=O)N1CCN(c2nc(OCCN(CC)CC)nc3c2CO[C@H](c2cccc4cccc(C)c24)C3)C[C@@H]1CC#N ; C=CC(=O)N1CCN(c2nc(OCCN(CC)CC)nc3c2CO[C@@H](c2cccc4cccc(C)c24)C3)C[C@@H]1CC#N]

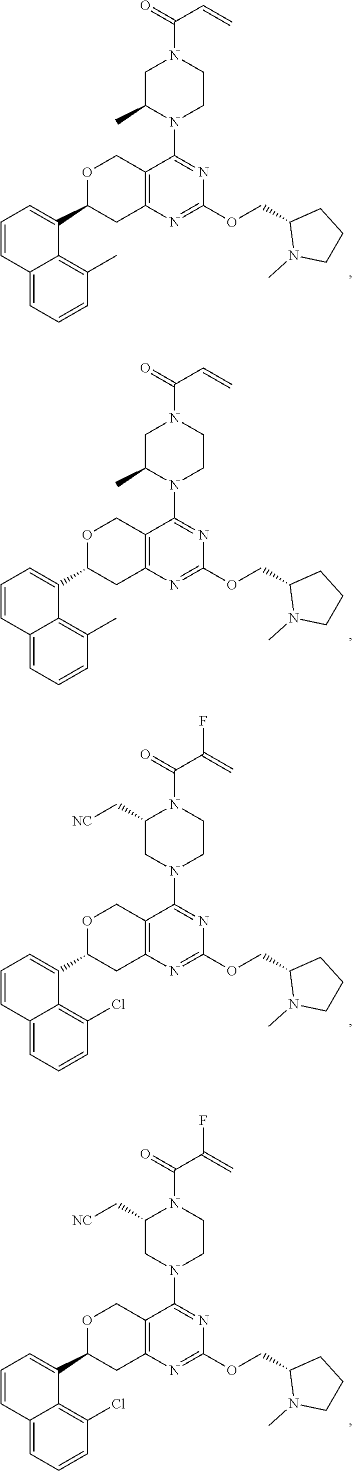 [SMILES: C=CC(=O)N1CCN(c2nc(OC[C@@H]3CCCN3C)nc3c2CO[C@H](c2cccc4cccc(C)c24)C3)[C@@H](C)C1 ; C=CC(=O)N1CCN(c2nc(OC[C@@H]3CCCN3C)nc3c2CO[C@@H](c2cccc4cccc(C)c24)C3)[C@@H](C)C1 ; C=C(F)C(=O)N1CCN(c2nc(OC[C@@H]3CCCN3C)nc3c2CO[C@@H](c2cccc4cccc(Cl)c24)C3)C[C@@H]1CC#N ; C=C(F)C(=O)N1CCN(c2nc(OC[C@@H]3CCCN3C)nc3c2CO[C@H](c2cccc4cccc(Cl)c24)C3)C[C@@H]1CC#N]

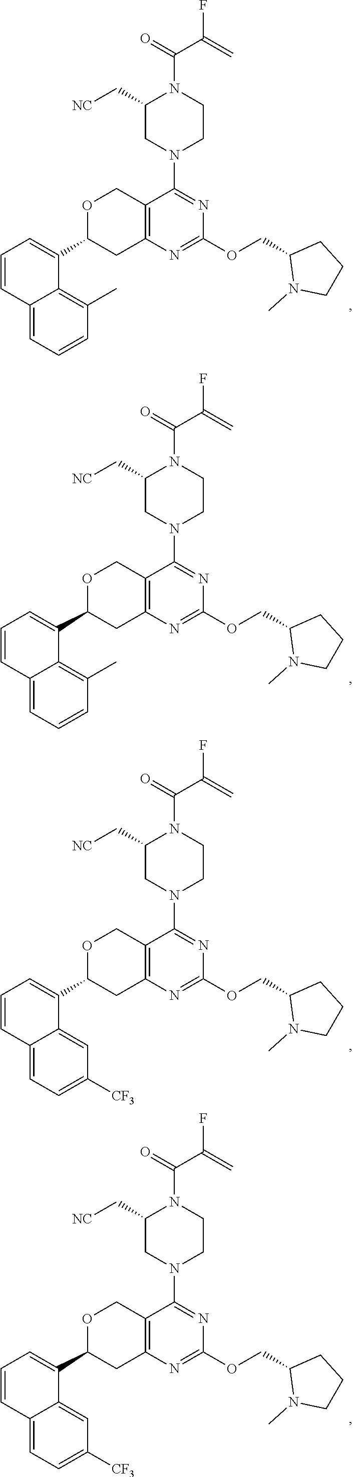 [SMILES: C=C(F)C(=O)N1CCN(c2nc(OC[C@@H]3CCCN3C)nc3c2CO[C@@H](c2cccc4cccc(C)c24)C3)C[C@@H]1CC#N ; C=C(F)C(=O)N1CCN(c2nc(OC[C@@H]3CCCN3C)nc3c2CO[C@H](c2cccc4cccc(C)c24)C3)C[C@@H]1CC#N ; C=C(F)C(=O)N1CCN(c2nc(OC[C@@H]3CCCN3C)nc3c2CO[C@@H](c2cccc4ccc(C(F)(F)F)cc24)C3)C[C@@H]1CC#N ; C=C(F)C(=O)N1CCN(c2nc(OC[C@@H]3CCCN3C)nc3c2CO[C@H](c2cccc4ccc(C(F)(F)F)cc24)C3)C[C@@H]1CC#N]

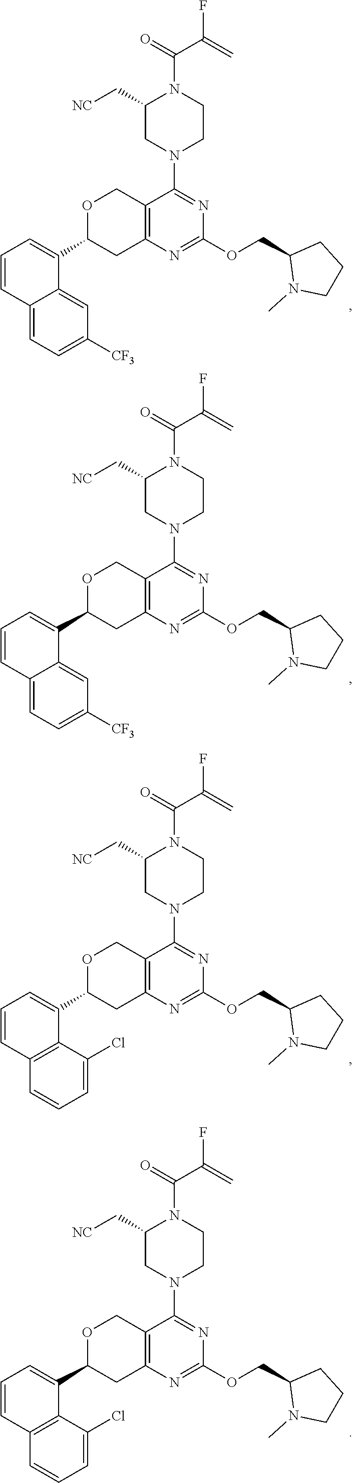 [SMILES: C=C(F)C(=O)N1CCN(c2nc(OC[C@H]3CCCN3C)nc3c2CO[C@@H](c2cccc4ccc(C(F)(F)F)cc24)C3)C[C@@H]1CC#N ; C=C(F)C(=O)N1CCN(c2nc(OC[C@H]3CCCN3C)nc3c2CO[C@H](c2cccc4ccc(C(F)(F)F)cc24)C3)C[C@@H]1CC#N ; C=C(F)C(=O)N1CCN(c2nc(OC[C@H]3CCCN3C)nc3c2CO[C@@H](c2cccc4cccc(Cl)c24)C3)C[C@@H]1CC#N ; C=C(F)C(=O)N1CCN(c2nc(OC[C@H]3CCCN3C)nc3c2CO[C@H](c2cccc4cccc(Cl)c24)C3)C[C@@H]1CC#N]

16. A method for preparing the oxygen-containing heterocyclic compound represented by formula I as defined in claim 1, the method comprising route one or route two:
route one: 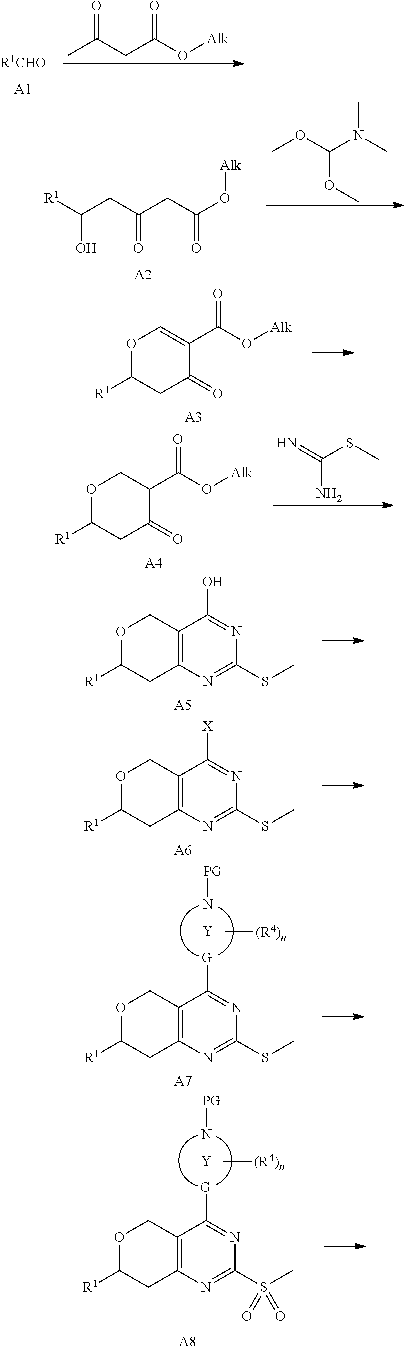 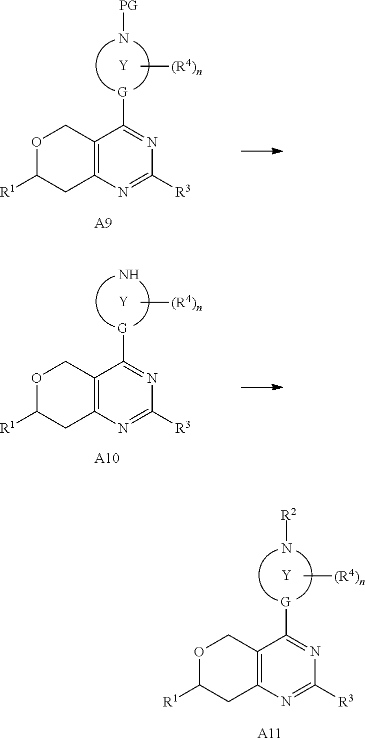
wherein, X₁ is a leaving group: Alk is alkyl; and PG is an amino protecting group;
route two: 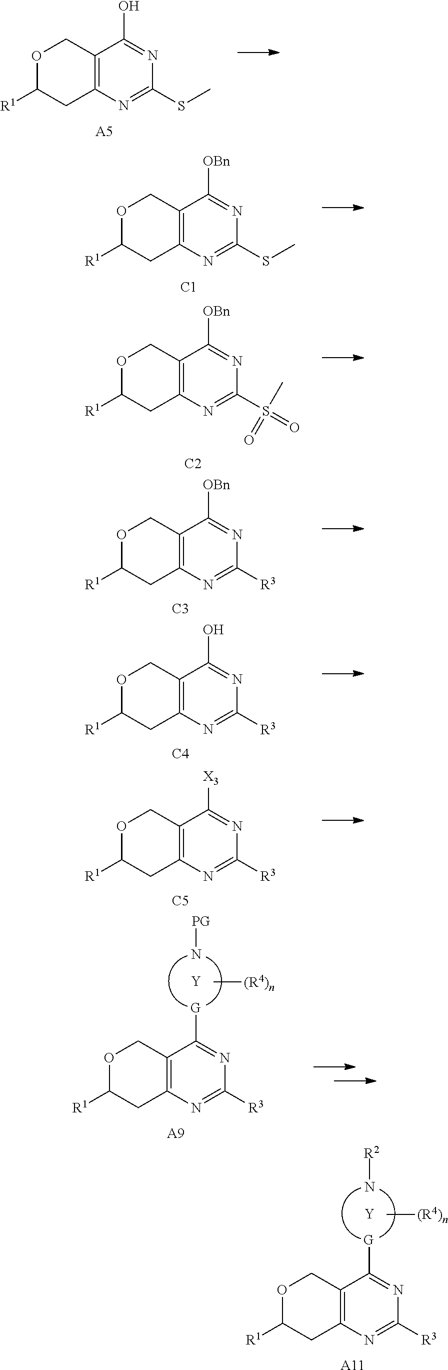
wherein, X₃ is a leaving group and PG is an amino protecting group.

17. A compound represented by formula A5, A6, A7, A8, A9, A10, C1, C2, C3, C4 or C5; 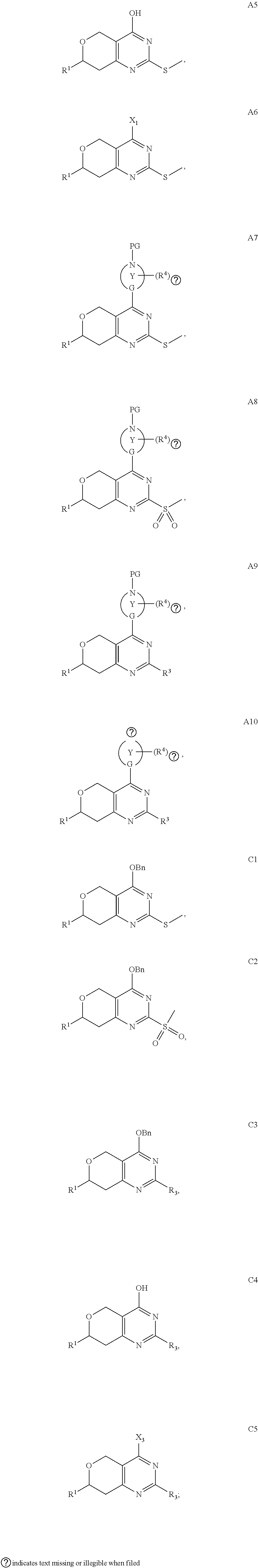
wherein R¹, R³, R⁴, G, Y and n are as defined in claim 1;
X¹ and X³ are independently leaving groups and PG is an amino protecting group; for example, the compound represented by formula A5, A6, A7, A8, A9, A10, C1, C2, C3, C4 or C5 is any one of the following compounds:

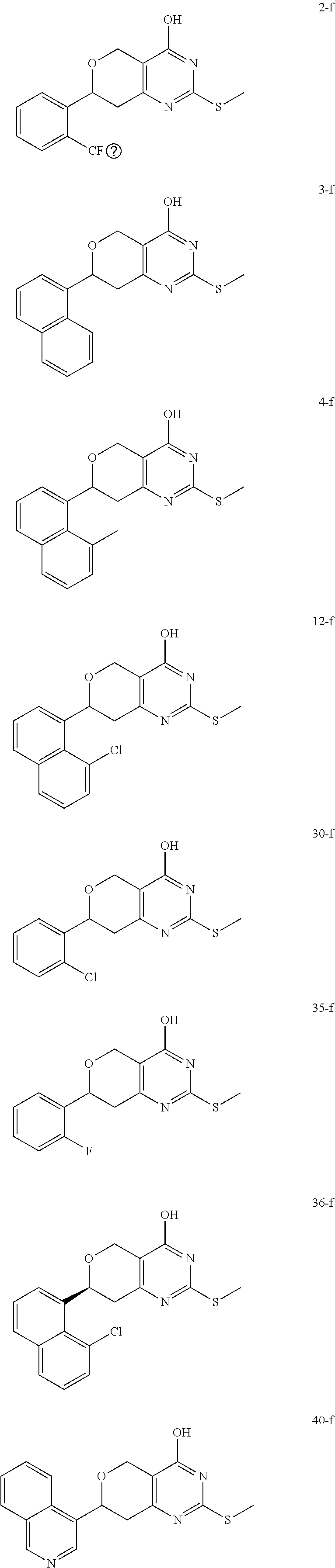

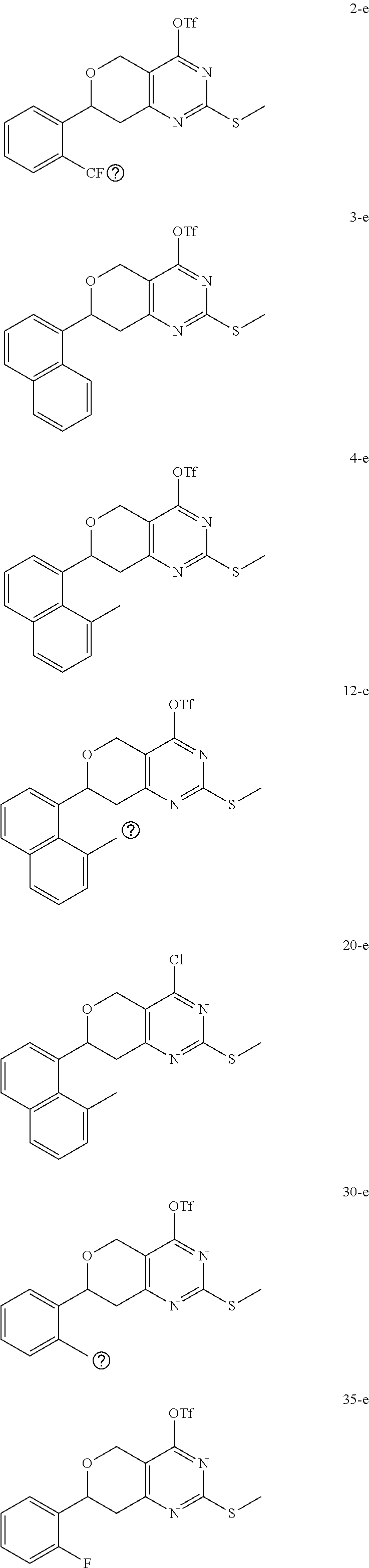

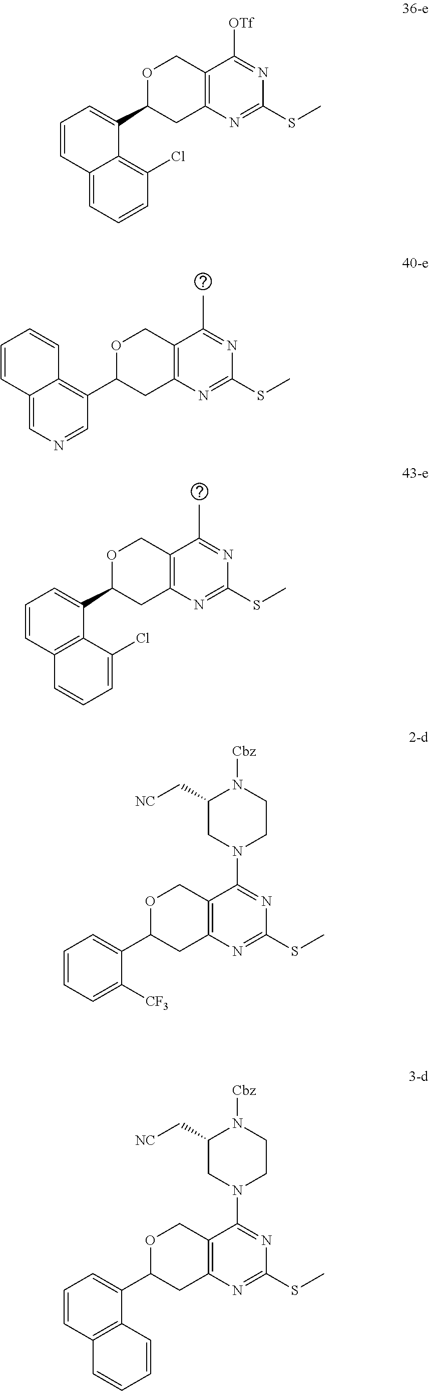 [SMILES: CSc1nc2c(c(OS(=O)(=O)C(F)(F)F)n1)CO[C@H](c1cccc3cccc(Cl)c13)C2 ; CSc1nc(C)c2c(n1)CC(c1cncc3ccccc13)OC2 ; CSc1nc(C)c2c(n1)C[C@@H](c1cccc3cccc(Cl)c13)OC2 ; CSc1nc2c(c(N3CCN(C(=O)OCc4ccccc4)[C@@H](CC#N)C3)n1)COC(c1ccccc1C(F)(F)F)C2 ; CSc1nc2c(c(N3CCN(C(=O)OCc4ccccc4)[C@@H](CC#N)C3)n1)COC(c1cccc3ccccc13)C2]

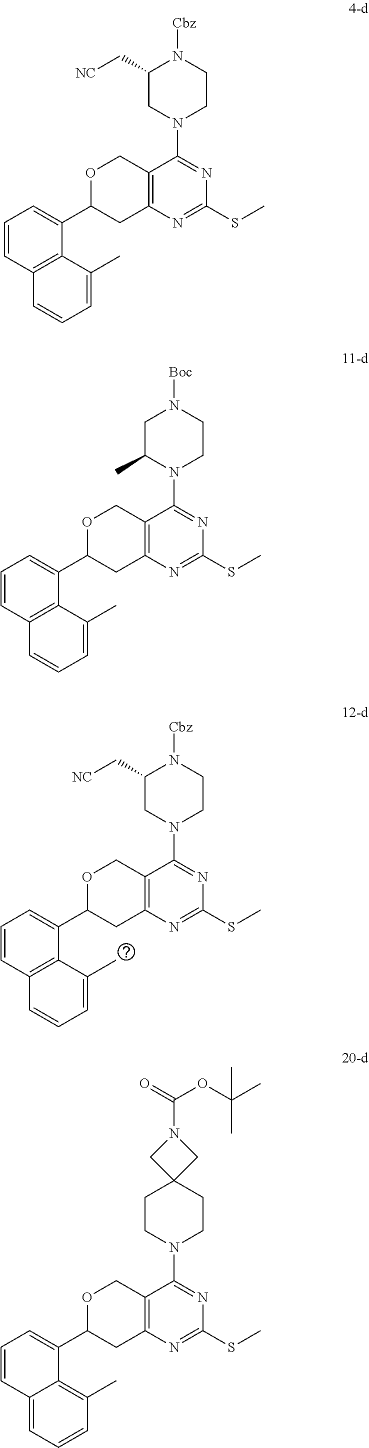 [SMILES: CSc1nc2c(c(N3CCN(C(=O)OCc4ccccc4)[C@@H](CC#N)C3)n1)COC(c1cccc3cccc(C)c13)C2 ; CSc1nc2c(c(N3CCN(C(=O)OC(C)(C)C)C[C@@H]3C)n1)COC(c1cccc3cccc(C)c13)C2 ; CSc1nc2c(c(N3CCN(C(=O)OCc4ccccc4)[C@@H](CC#N)C3)n1)COC(c1cccc3cccc(C)c13)C2 ; CSc1nc2c(c(N3CCC4(CC3)CN(C(=O)OC(C)(C)C)C4)n1)COC(c1cccc3cccc(C)c13)C2]

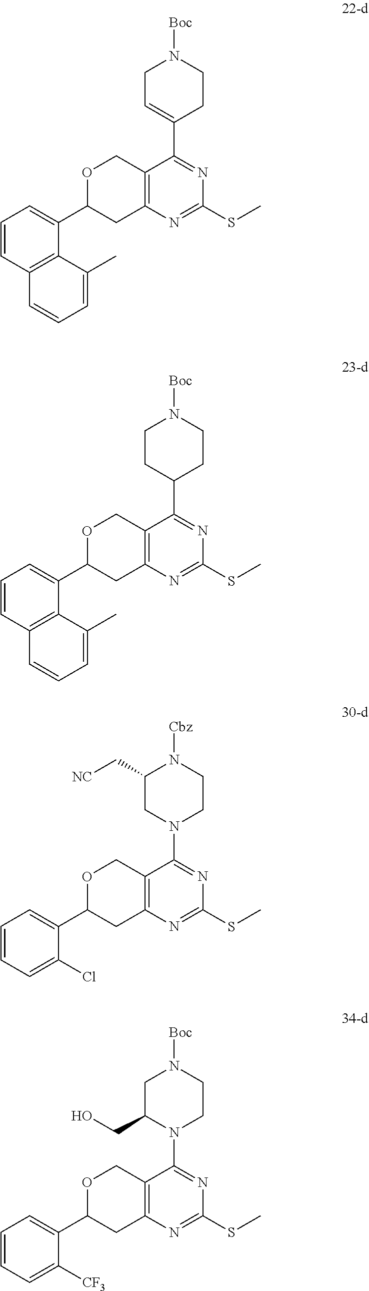 [SMILES: CSc1nc2c(c(C3=CCN(C(=O)OC(C)(C)C)CC3)n1)COC(c1cccc3cccc(C)c13)C2 ; CSc1nc2c(c(C3CCN(C(=O)OC(C)(C)C)CC3)n1)COC(c1cccc3cccc(C)c13)C2 ; CSc1nc2c(c(N3CCN(C(=O)OCc4ccccc4)[C@@H](CC#N)C3)n1)COC(c1ccccc1Cl)C2 ; CSc1nc2c(c(N3CCN(C(=O)OC(C)(C)C)C[C@@H]3CO)n1)COC(c1ccccc1C(F)(F)F)C2]

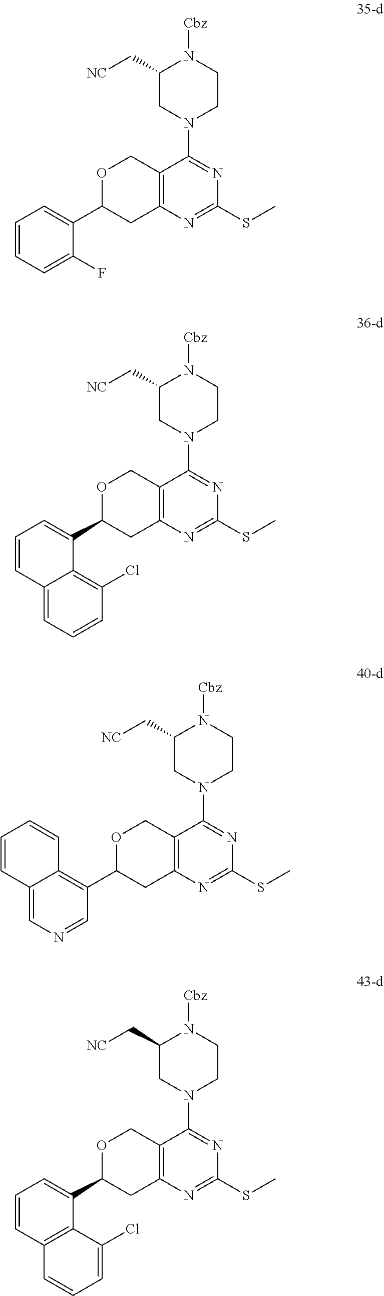 [SMILES: CSc1nc2c(c(N3CCN(C(=O)OCc4ccccc4)[C@@H](CC#N)C3)n1)COC(c1ccccc1F)C2 ; CSc1nc2c(c(N3CCN(C(=O)OCc4ccccc4)[C@@H](CC#N)C3)n1)CO[C@H](c1cccc3cccc(Cl)c13)C2 ; CSc1nc2c(c(N3CCN(C(=O)OCc4ccccc4)[C@@H](CC#N)C3)n1)COC(c1cncc3ccccc13)C2 ; CSc1nc2c(c(N3CCN(C(=O)OCc4ccccc4)[C@H](CC#N)C3)n1)CO[C@H](c1cccc3cccc(Cl)c13)C2]

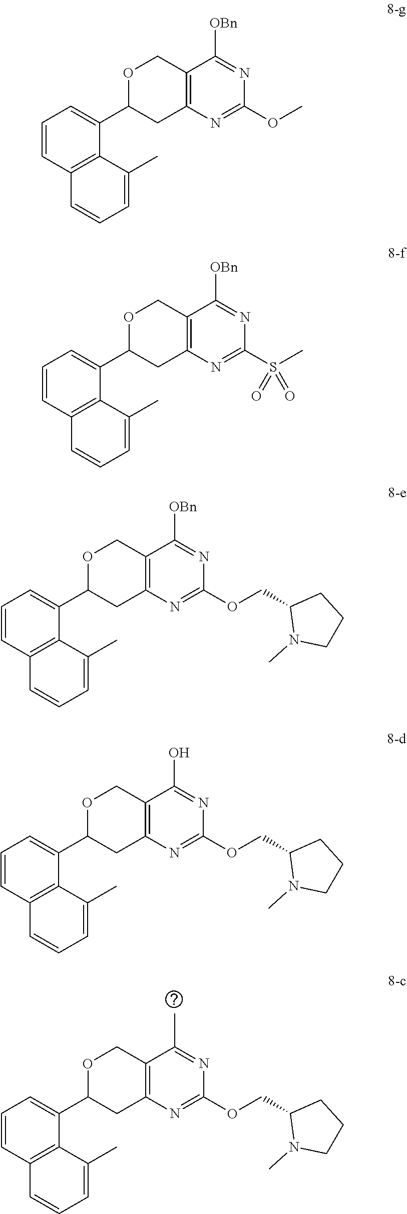 [SMILES: COc1nc2c(c(OCc3ccccc3)n1)COC(c1cccc3cccc(C)c13)C2 ; Cc1cccc2cccc(C3Cc4nc(S(C)(=O)=O)nc(OCc5ccccc5)c4CO3)c12 ; Cc1cccc2cccc(C3Cc4nc(OC[C@@H]5CCCN5C)nc(OCc5ccccc5)c4CO3)c12 ; Cc1cccc2cccc(C3Cc4nc(OC[C@@H]5CCCN5C)nc(O)c4CO3)c12 ; Cc1nc(OC[C@@H]2CCCN2C)nc2c1COC(c1cccc3cccc(C)c13)C2]

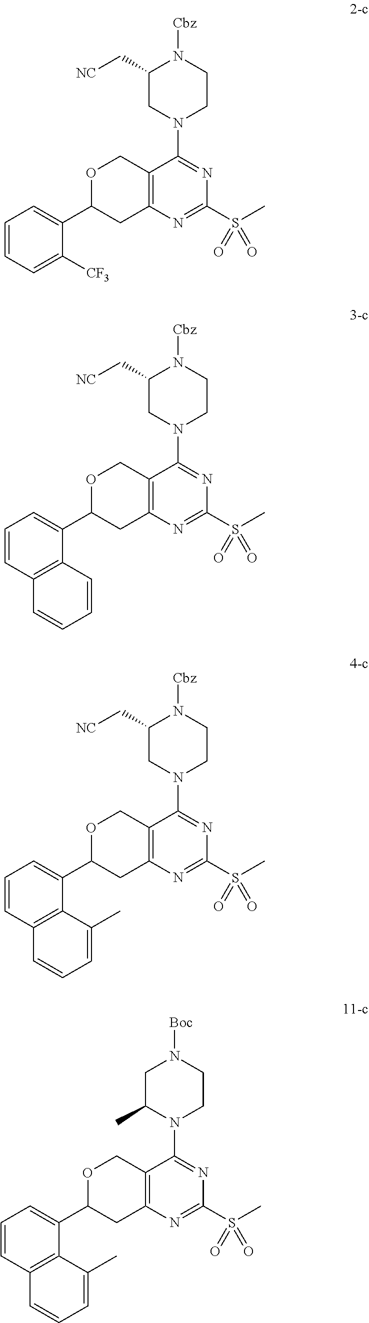 [SMILES: CS(=O)(=O)c1nc2c(c(N3CCN(C(=O)OCc4ccccc4)[C@@H](CC#N)C3)n1)COC(c1ccccc1C(F)(F)F)C2 ; CS(=O)(=O)c1nc2c(c(N3CCN(C(=O)OCc4ccccc4)[C@@H](CC#N)C3)n1)COC(c1cccc3ccccc13)C2 ; Cc1cccc2cccc(C3Cc4nc(S(C)(=O)=O)nc(N5CCN(C(=O)OCc6ccccc6)[C@@H](CC#N)C5)c4CO3)c12 ; Cc1cccc2cccc(C3Cc4nc(S(C)(=O)=O)nc(N5CCN(C(=O)OC(C)(C)C)C[C@@H]5C)c4CO3)c12]

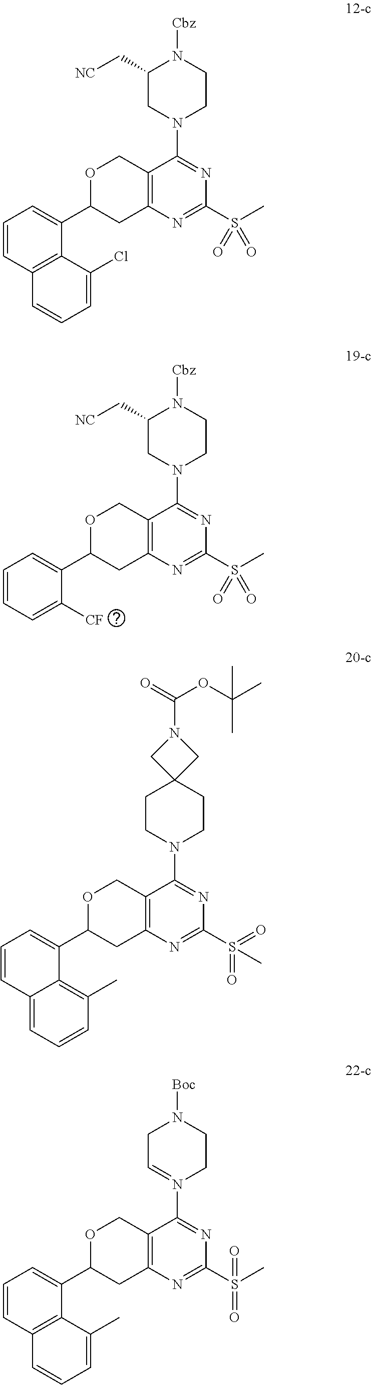

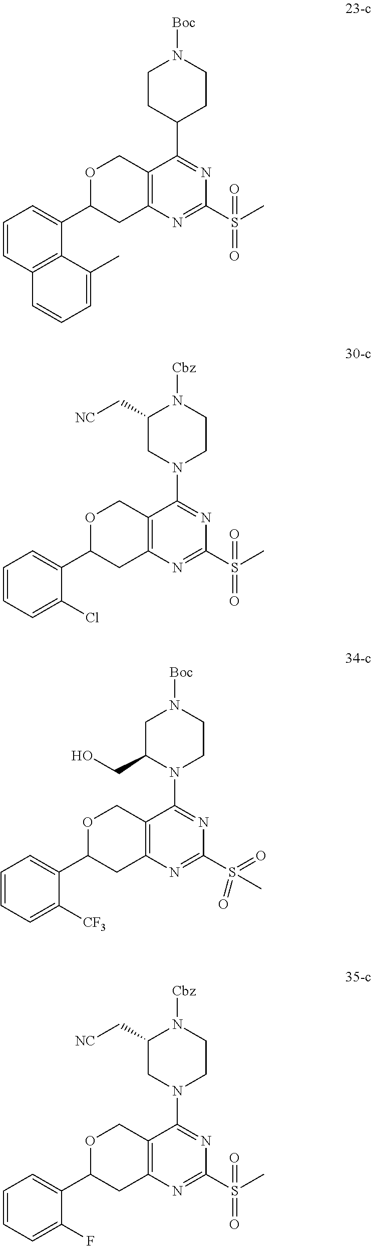 [SMILES: Cc1cccc2cccc(C3Cc4nc(S(C)(=O)=O)nc(C5CCN(C(=O)OC(C)(C)C)CC5)c4CO3)c12 ; CS(=O)(=O)c1nc2c(c(N3CCN(C(=O)OCc4ccccc4)[C@@H](CC#N)C3)n1)COC(c1ccccc1Cl)C2 ; CC(C)(C)OC(=O)N1CCN(c2nc(S(C)(=O)=O)nc3c2COC(c2ccccc2C(F)(F)F)C3)[C@@H](CO)C1 ; CS(=O)(=O)c1nc2c(c(N3CCN(C(=O)OCc4ccccc4)[C@@H](CC#N)C3)n1)COC(c1ccccc1F)C2]

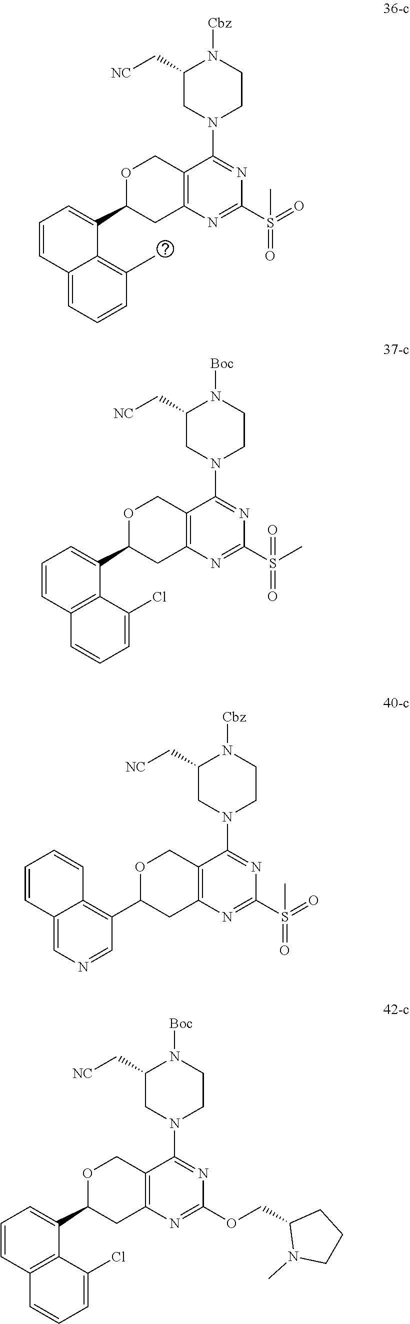 [SMILES: Cc1cccc2cccc([C@@H]3Cc4nc(S(C)(=O)=O)nc(N5CCN(C(=O)OCc6ccccc6)[C@@H](CC#N)C5)c4CO3)c12 ; CC(C)(C)OC(=O)N1CCN(c2nc(S(C)(=O)=O)nc3c2CO[C@H](c2cccc4cccc(Cl)c24)C3)C[C@@H]1CC#N ; CS(=O)(=O)c1nc2c(c(N3CCN(C(=O)OCc4ccccc4)[C@@H](CC#N)C3)n1)COC(c1cncc3ccccc13)C2 ; CN1CCC[C@H]1COc1nc2c(c(N3CCN(C(=O)OC(C)(C)C)[C@@H](CC#N)C3)n1)CO[C@H](c1cccc3cccc(Cl)c13)C2]

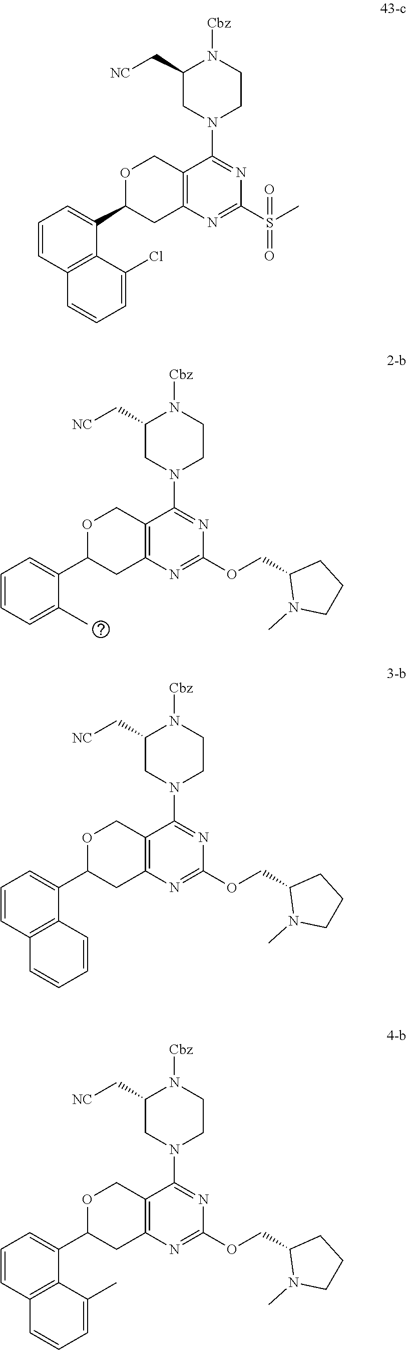 [SMILES: CS(=O)(=O)c1nc2c(c(N3CCN(C(=O)OCc4ccccc4)[C@H](CC#N)C3)n1)CO[C@H](c1cccc3cccc(Cl)c13)C2 ; Cc1ccccc1C1Cc2nc(OC[C@@H]3CCCN3C)nc(N3CCN(C(=O)OCc4ccccc4)[C@@H](CC#N)C3)c2CO1 ; CN1CCC[C@H]1COc1nc2c(c(N3CCN(C(=O)OCc4ccccc4)[C@@H](CC#N)C3)n1)COC(c1cccc3ccccc13)C2 ; Cc1cccc2cccc(C3Cc4nc(OC[C@@H]5CCCN5C)nc(N5CCN(C(=O)OCc6ccccc6)[C@@H](CC#N)C5)c4CO3)c12]

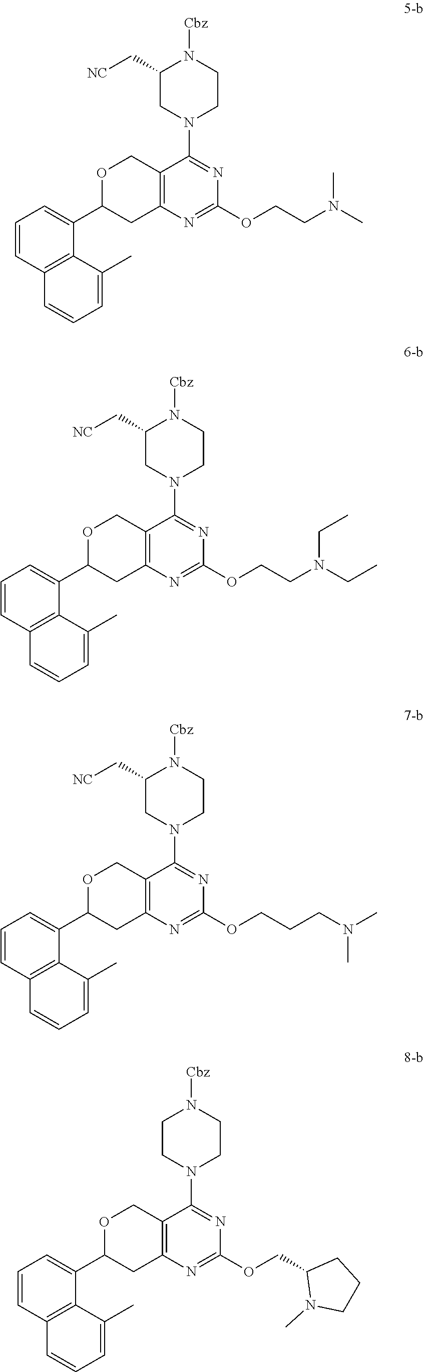 [SMILES: Cc1cccc2cccc(C3Cc4nc(OCCN(C)C)nc(N5CCN(C(=O)OCc6ccccc6)[C@@H](CC#N)C5)c4CO3)c12 ; CCN(CC)CCOc1nc2c(c(N3CCN(C(=O)OCc4ccccc4)[C@@H](CC#N)C3)n1)COC(c1cccc3cccc(C)c13)C2 ; Cc1cccc2cccc(C3Cc4nc(OCCCN(C)C)nc(N5CCN(C(=O)OCc6ccccc6)[C@@H](CC#N)C5)c4CO3)c12 ; Cc1cccc2cccc(C3Cc4nc(OC[C@@H]5CCCN5C)nc(N5CCN(C(=O)OCc6ccccc6)CC5)c4CO3)c12]

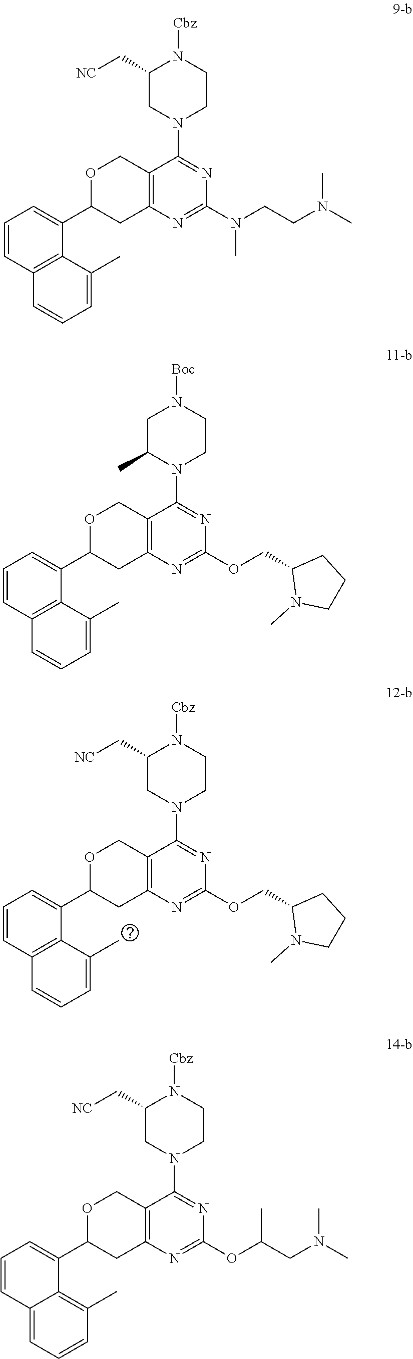 [SMILES: Cc1cccc2cccc(C3Cc4nc(N(C)CCN(C)C)nc(N5CCN(C(=O)OCc6ccccc6)[C@@H](CC#N)C5)c4CO3)c12 ; Cc1cccc2cccc(C3Cc4nc(OC[C@@H]5CCCN5C)nc(N5CCN(C(=O)OC(C)(C)C)C[C@@H]5C)c4CO3)c12 ; Cc1cccc2cccc(C3Cc4nc(OC[C@@H]5CCCN5C)nc(N5CCN(C(=O)OCc6ccccc6)[C@@H](CC#N)C5)c4CO3)c12 ; Cc1cccc2cccc(C3Cc4nc(OC(C)CN(C)C)nc(N5CCN(C(=O)OCc6ccccc6)[C@@H](CC#N)C5)c4CO3)c12]

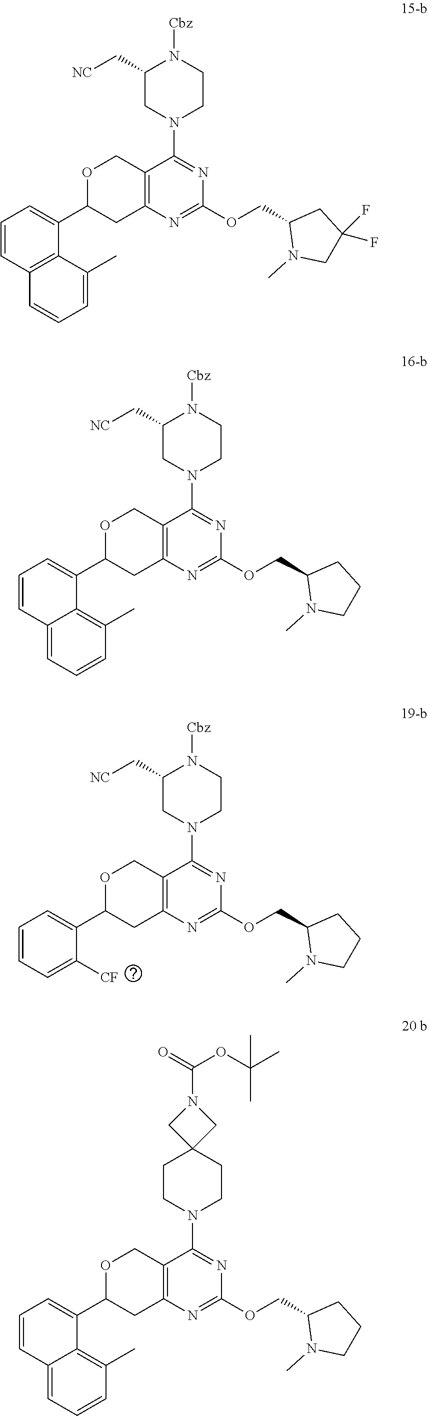

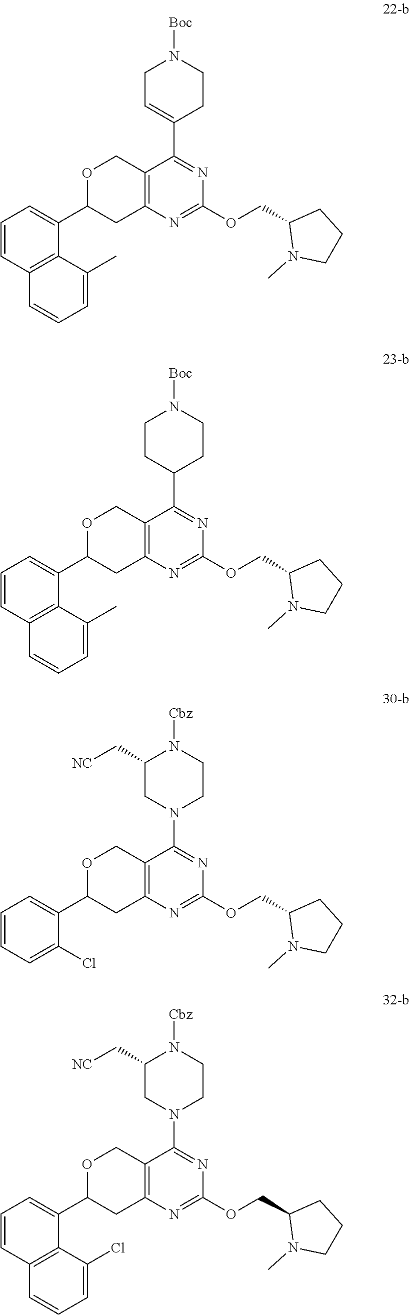 [SMILES: Cc1cccc2cccc(C3Cc4nc(OC[C@@H]5CCCN5C)nc(C5=CCN(C(=O)OC(C)(C)C)CC5)c4CO3)c12 ; Cc1cccc2cccc(C3Cc4nc(OC[C@@H]5CCCN5C)nc(C5CCN(C(=O)OC(C)(C)C)CC5)c4CO3)c12 ; CN1CCC[C@H]1COc1nc2c(c(N3CCN(C(=O)OCc4ccccc4)[C@@H](CC#N)C3)n1)COC(c1ccccc1Cl)C2 ; CN1CCC[C@@H]1COc1nc2c(c(N3CCN(C(=O)OCc4ccccc4)[C@@H](CC#N)C3)n1)COC(c1cccc3cccc(Cl)c13)C2]

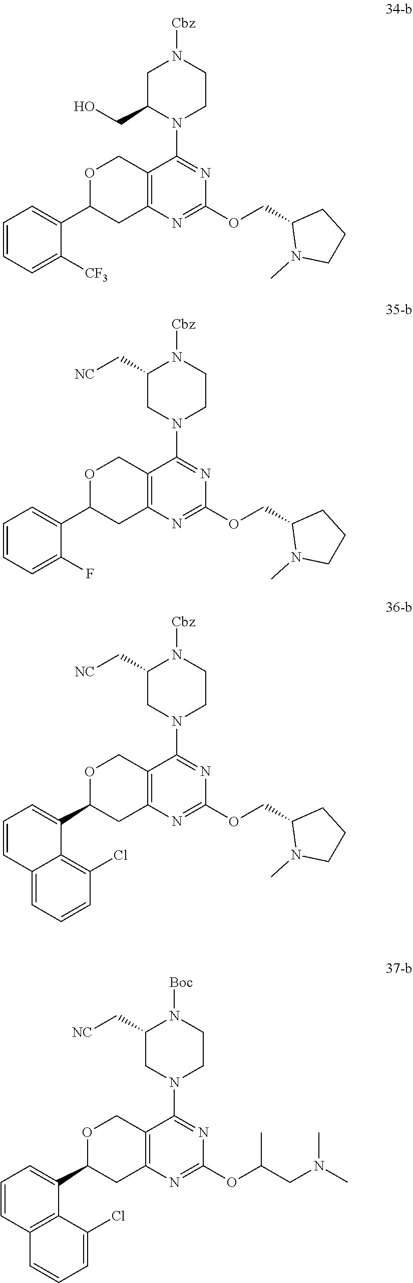 [SMILES: CN1CCC[C@H]1COc1nc2c(c(N3CCN(C(=O)OCc4ccccc4)C[C@@H]3CO)n1)COC(c1ccccc1C(F)(F)F)C2 ; CN1CCC[C@H]1COc1nc2c(c(N3CCN(C(=O)OCc4ccccc4)[C@@H](CC#N)C3)n1)COC(c1ccccc1F)C2 ; CN1CCC[C@H]1COc1nc2c(c(N3CCN(C(=O)OCc4ccccc4)[C@@H](CC#N)C3)n1)CO[C@H](c1cccc3cccc(Cl)c13)C2 ; CC(CN(C)C)Oc1nc2c(c(N3CCN(C(=O)OC(C)(C)C)[C@@H](CC#N)C3)n1)CO[C@H](c1cccc3cccc(Cl)c13)C2]

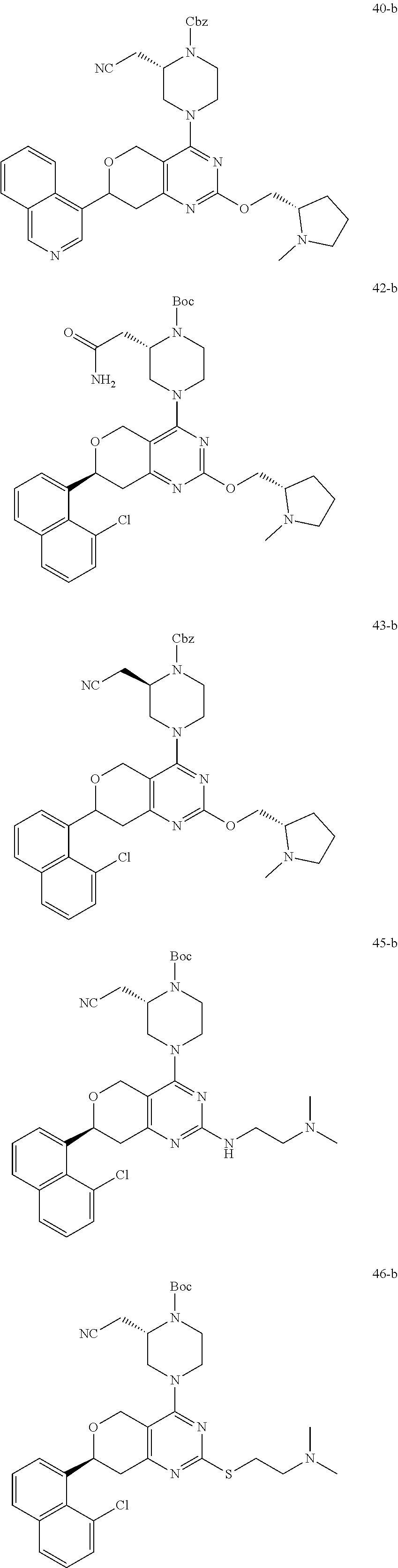 [SMILES: CN1CCC[C@H]1COc1nc2c(c(N3CCN(C(=O)OCc4ccccc4)[C@@H](CC#N)C3)n1)COC(c1cncc3ccccc13)C2 ; CN1CCC[C@H]1COc1nc2c(c(N3CCN(C(=O)OC(C)(C)C)[C@@H](CC(N)=O)C3)n1)CO[C@H](c1cccc3cccc(Cl)c13)C2 ; CN1CCC[C@H]1COc1nc2c(c(N3CCN(C(=O)OCc4ccccc4)[C@H](CC#N)C3)n1)COC(c1cccc3cccc(Cl)c13)C2 ; CN(C)CCNc1nc2c(c(N3CCN(C(=O)OC(C)(C)C)[C@@H](CC#N)C3)n1)CO[C@H](c1cccc3cccc(Cl)c13)C2 ; CN(C)CCSc1nc2c(c(N3CCN(C(=O)OC(C)(C)C)[C@@H](CC#N)C3)n1)CO[C@H](c1cccc3cccc(Cl)c13)C2]

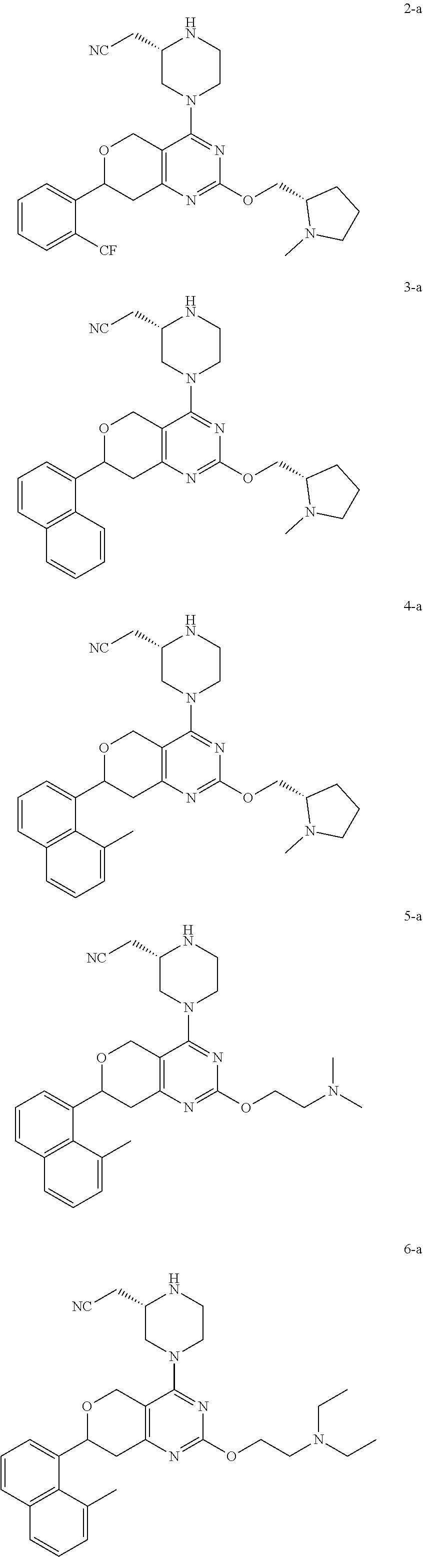

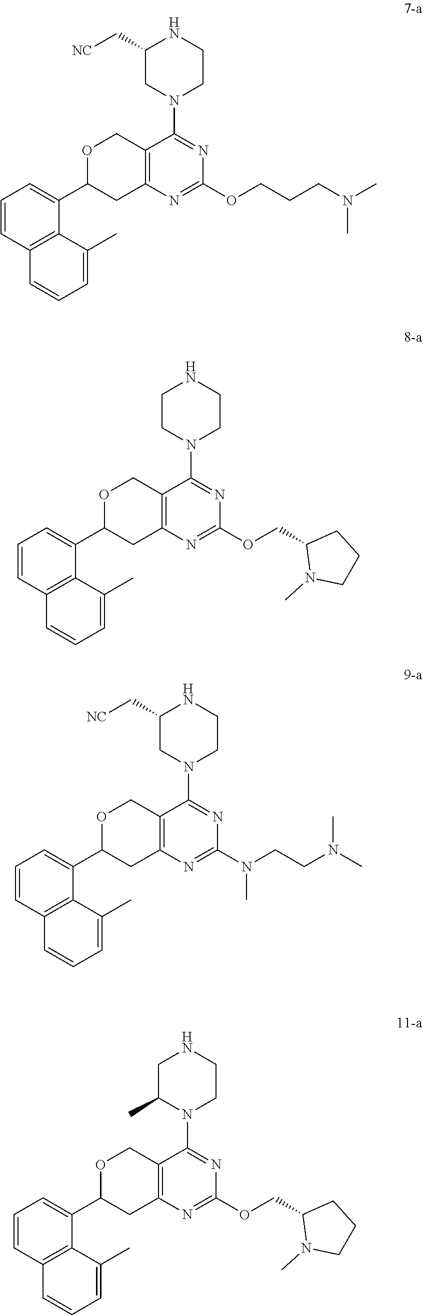 [SMILES: Cc1cccc2cccc(C3Cc4nc(OCCCN(C)C)nc(N5CCN[C@@H](CC#N)C5)c4CO3)c12 ; Cc1cccc2cccc(C3Cc4nc(OC[C@@H]5CCCN5C)nc(N5CCNCC5)c4CO3)c12 ; Cc1cccc2cccc(C3Cc4nc(N(C)CCN(C)C)nc(N5CCN[C@@H](CC#N)C5)c4CO3)c12 ; Cc1cccc2cccc(C3Cc4nc(OC[C@@H]5CCCN5C)nc(N5CCNC[C@@H]5C)c4CO3)c12]

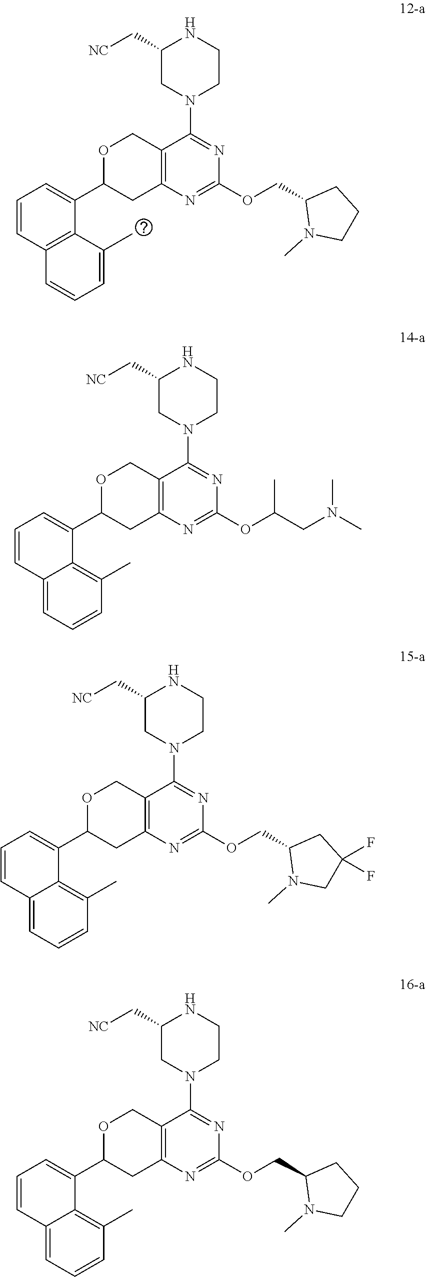 [SMILES: Cc1cccc2cccc(C3Cc4nc(OC[C@@H]5CCCN5C)nc(N5CCN[C@@H](CC#N)C5)c4CO3)c12 ; Cc1cccc2cccc(C3Cc4nc(OC(C)CN(C)C)nc(N5CCN[C@@H](CC#N)C5)c4CO3)c12 ; Cc1cccc2cccc(C3Cc4nc(OC[C@@H]5CC(F)(F)CN5C)nc(N5CCN[C@@H](CC#N)C5)c4CO3)c12 ; Cc1cccc2cccc(C3Cc4nc(OC[C@H]5CCCN5C)nc(N5CCN[C@@H](CC#N)C5)c4CO3)c12]

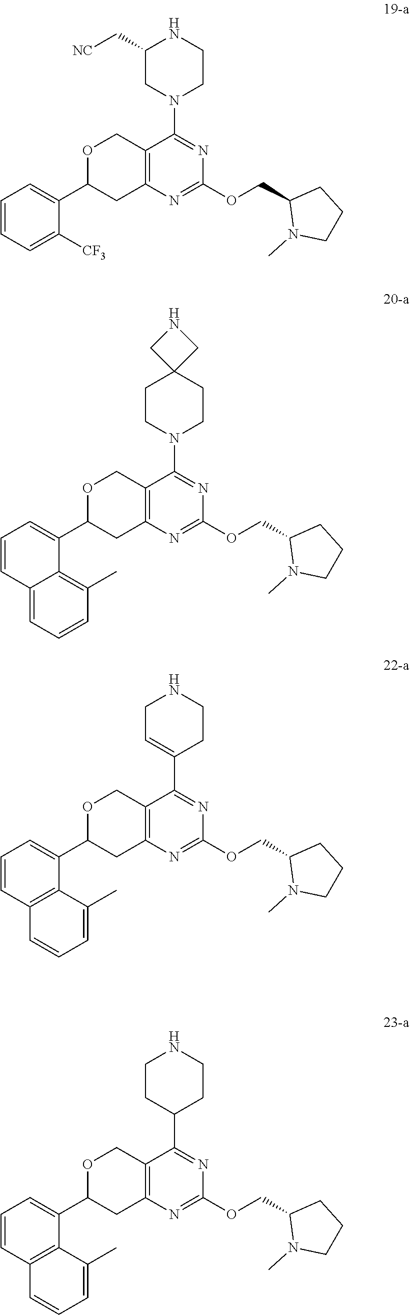 [SMILES: CN1CCC[C@@H]1COc1nc2c(c(N3CCN[C@@H](CC#N)C3)n1)COC(c1ccccc1C(F)(F)F)C2 ; Cc1cccc2cccc(C3Cc4nc(OC[C@@H]5CCCN5C)nc(N5CCC6(CC5)CNC6)c4CO3)c12 ; Cc1cccc2cccc(C3Cc4nc(OC[C@@H]5CCCN5C)nc(C5=CCNCC5)c4CO3)c12 ; Cc1cccc2cccc(C3Cc4nc(OC[C@@H]5CCCN5C)nc(C5CCNCC5)c4CO3)c12]

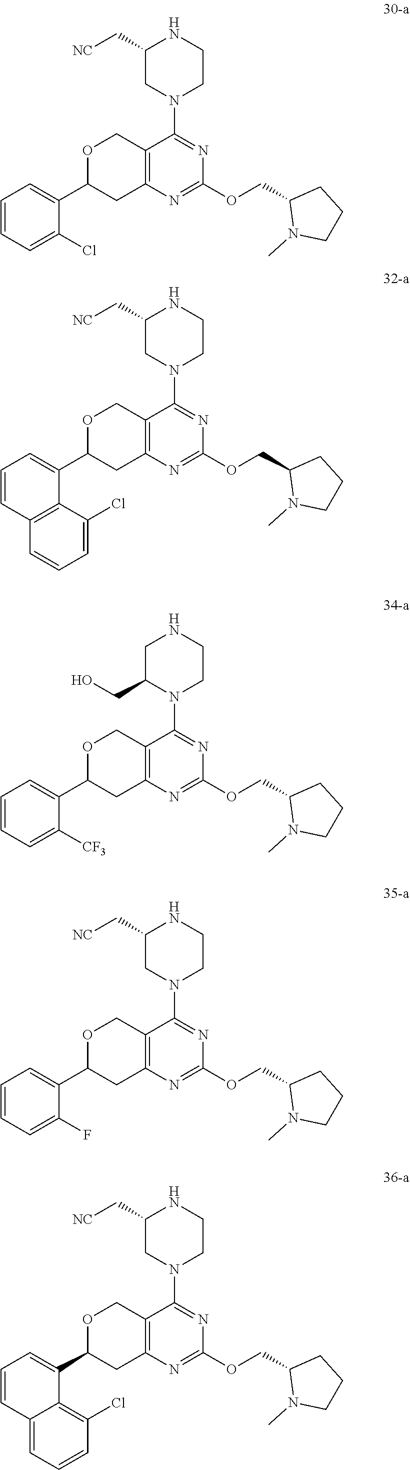 [SMILES: CN1CCC[C@H]1COc1nc2c(c(N3CCN[C@@H](CC#N)C3)n1)COC(c1ccccc1Cl)C2 ; CN1CCC[C@@H]1COc1nc2c(c(N3CCN[C@@H](CC#N)C3)n1)COC(c1cccc3cccc(Cl)c13)C2 ; CN1CCC[C@H]1COc1nc2c(c(N3CCNC[C@@H]3CO)n1)COC(c1ccccc1C(F)(F)F)C2 ; CN1CCC[C@H]1COc1nc2c(c(N3CCN[C@@H](CC#N)C3)n1)COC(c1ccccc1F)C2 ; CN1CCC[C@H]1COc1nc2c(c(N3CCN[C@@H](CC#N)C3)n1)CO[C@H](c1cccc3cccc(Cl)c13)C2]

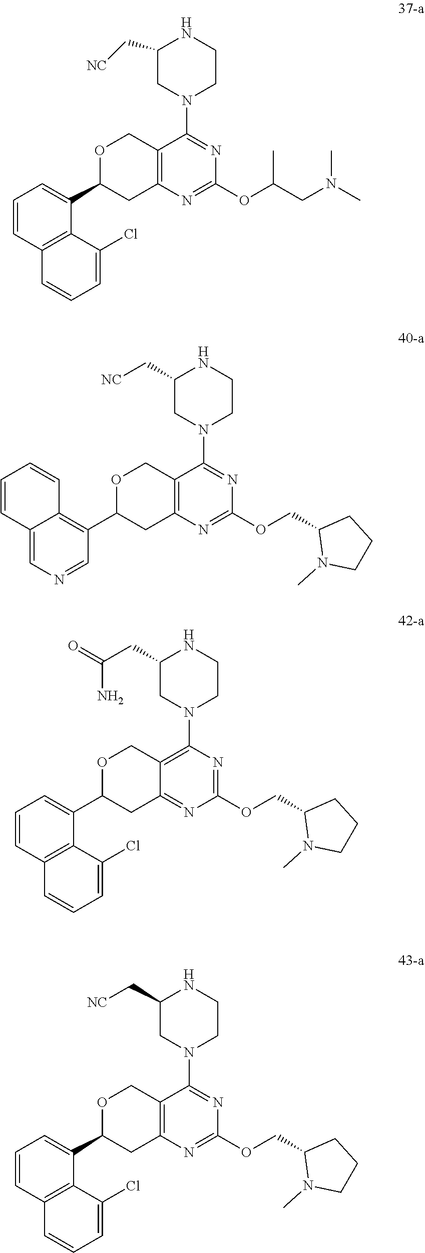 [SMILES: CC(CN(C)C)Oc1nc2c(c(N3CCN[C@@H](CC#N)C3)n1)CO[C@H](c1cccc3cccc(Cl)c13)C2 ; CN1CCC[C@H]1COc1nc2c(c(N3CCN[C@@H](CC#N)C3)n1)COC(c1cncc3ccccc13)C2 ; CN1CCC[C@H]1COc1nc2c(c(N3CCN[C@@H](CC(N)=O)C3)n1)COC(c1cccc3cccc(Cl)c13)C2 ; CN1CCC[C@H]1COc1nc2c(c(N3CCN[C@H](CC#N)C3)n1)CO[C@H](c1cccc3cccc(Cl)c13)C2]

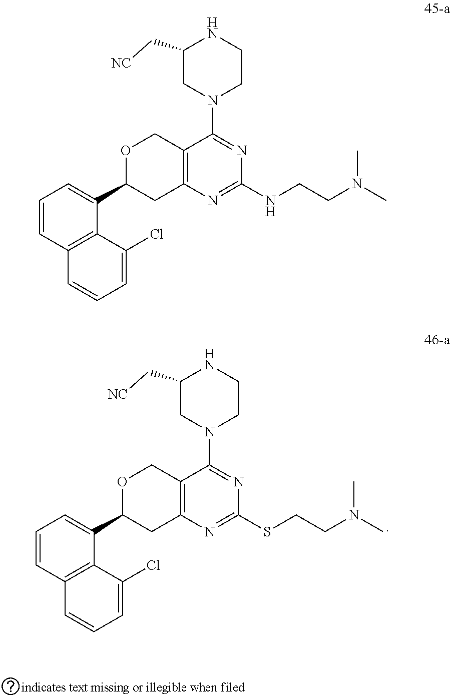 [SMILES: CN(C)CCNc1nc2c(c(N3CCN[C@@H](CC#N)C3)n1)CO[C@H](c1cccc3cccc(Cl)c13)C2 ; CN(C)CCSc1nc2c(c(N3CCN[C@@H](CC#N)C3)n1)CO[C@H](c1cccc3cccc(Cl)c13)C2]

18. A pharmaceutical composition, comprising substance A and a pharmaceutical adjuvant: the substance A is the oxygen-containing heterocyclic compound represented by formula I as defined in claim 1, a pharmaceutically acceptable salt thereof, a solvate thereof, a solvate of the pharmaceutically acceptable salt thereof, a crystal form thereof, a stereoisomer thereof, a tautomer thereof or an isotopic compound thereof.

19. (canceled)

20. A method for inhibiting RAS or for treating or preventing an RAS-mediated disease, or for treating or preventing cancer, the method comprising administrating to a patient a therapeutically effective amount of substance A;
the substance A is the oxygen-containing heterocyclic compound represented by formula I as defined in claim 1, a pharmaceutically acceptable salt thereof, a solvate thereof, a solvate of the pharmaceutically acceptable salt thereof, a crystal form thereof, a stereoisomer thereof, a tautomer thereof or an isotopic compound thereof;
the RAS-mediated disease is, for example, cancer:
the cancer is, for example, one or more of colon cancer, pancreatic cancer, breast cancer, prostate cancer, lung cancer, brain cancer, ovarian cancer, cervical cancer, testicular cancer, renal carcinoma, head or neck cancer, bone cancer, skin cancer, rectal cancer, liver cancer, colon cancer, esophageal cancer, gastric cancer, pancreatic cancer, thyroid cancer, bladder cancer, lymphoma, leukemia and melanoma.
